(19) United States
(12) Patent Application Publication
(10) Pub. No.: US 2023/0250479 A1
(43) Pub. Date: Aug. 10, 2023

(54) METHOD FOR DETERMINING THE RISK OF INCIDENCE OF A CARE-RELATED INFECTION IN A PATIENT

(71) Applicants: BIOMÉRIEUX, Marcy L'Etoile (FR); BIOASTER, Lyon (FR)

(72) Inventors: Laurence VACHOT, Fontaine (FR); François MALLET, Villeurbanne (FR); Guillaume MONNERET, Lyon (FR); Virginie MOUCADEL, Sassenage (FR); Alexandre PACHOT, Sulignat (FR); Estelle PERONNET, Lyon (FR); Thomas RIMMELÉ, Lyon (FR); Julien TEXTORIS, Villeurbanne (FR); Fabienne VENET, Lyon (FR)

(73) Assignees: BIOMÉRIEUX, Marcy L'Etoile (FR); BIOASTER, Lyon (FR)

(21) Appl. No.: 18/014,885

(22) PCT Filed: Jul. 5, 2021
(86) PCT No.: PCT/FR2021/051230

§ 371 Date: Jan. 6, 2023

(30) Foreign Application Priority Data
Jul. 6, 2020 (FR) FR2007134

(51) Int. Cl. C12Q 1/6883

(52) U.S. Cl. CPC C12Q 1/6883; C12Q 2600/118; C12Q 2600/158

(57) ABSTRACT

An in vitro or ex vivo method for determining the risk of incidence of a care-related infection includes a step of measuring the expression of CD177 in a biological sample from said patient.

The present invention relates to an in vitro or ex vivo method for determining the risk of incidence of a healthcare-associated infection, comprising a step of measuring the expression of CD177 in a biological sample from said patient.

The development of healthcare-associated infections is a significant complication associated with medical care, in particular in medical care facilities such as hospitals (where reference is more specifically made to nosocomial infections). It has been demonstrated that nosocomial infections in intensive care units, which occur in 20 to 40% of patients, were associated with increased morbidity and mortality, a longer requirement for supplemental care for organ failure(s), a longer stay in hospital, higher healthcare costs, and greater use of antibiotics, contributing to antimicrobial resistance. The incidence of healthcare-associated infections has become particularly exacerbated in recent years, due to the increase in multi-drug resistant pathogens. The World Health Organization (WHO) estimates the number of nosocomial infections in European hospitals to be around 5 million, leading to approximately 50 000 deaths and an annual excess cost of 13 to 24 billion Euros. Numerous factors influence the incidence and development of healthcare-associated infections, such as the patient's general state of health, but also factors associated with the patient's treatment (e.g. the administration of antibiotics and/or the use of invasive medical devices), factors associated with the hospital environment (e.g. the ratio of the number of nurses to the number of patients), and the varying use of aseptic techniques by hospital personnel. Recommendations have been published, and the establishment of infection control programs has been encouraged, in particular by the U.S. Department of Health and Human Services, the European Center for Disease Prevention and Control, the WHO and national agencies, for which the prevention and reduction of healthcare-associated infections have become a major priority. It has been shown that programs for controlling healthcare-associated infections have proven particularly effective in reducing serious infections. However, it has been estimated that a maximum of 65 to 70% of cases of blood and urinary tract infections, associated with catheterization, and 55% of cases of pneumonia, associated with mechanical ventilation, and of infections at surgical sites, could be prevented. Moreover, compliance with and application of procedures according to recommendations can be complicated in some hospitals, particularly in low- or middle-income countries. The early identification of patients at risk of developing a healthcare-associated infection would be a key step in preventing these infections and in treating these patients. According to certain models, a biomarker which would reduce the identification time of healthcare-associated infections in a high-risk population would make it possible to reduce mortality in these patients, with a good benefit-cost ratio. However, there is currently no clinical in vitro diagnostic test which makes it possible to identify patients with a high risk of contracting a healthcare-associated infection.

The CD177 gene encodes the membrane glycoprotein of CD177 neutrophils, and was discovered in 1971 in a case of neonatal neutropenia. A study has demonstrated an increase in the expression level of CD177 at the mRNA and protein level in circulating neutrophils from patients who have suffered septic shock, without however any association with commonly used immunosuppression markers such as the expression of HLA-DR at the surface of monocytes, the percentage of regulatory T cells or the number of CD4+ T cells (Demaret et al. (2016), Immunology Letters 178:122-130). In another study it was shown that the mRNA expression level of CD177 made it possible to distinguish patients who had suffered septic shock from healthy volunteers and, in combination with other biomarkers, to classify the septic patients into two categories (Schaack et al. (2018), PLoS One 13(6):e0198555). It has also been shown that the expression level of mRNA encoding the CD177 protein, among other biomarkers, made it possible to distinguish patients with community-acquired pneumonia from patients without same (Scicluna et al. (2015), Am J Respir Crit Care Med 192(7):826-35). Moreover, in patients who had undergone surgery, it was recently shown that certain genes, including CD177, were over-expressed in the non-surviving group (Martinez-Paz et al. (2020), J. Clin. Med. 9:1276).

It has now been discovered, entirely surprisingly, that measuring the expression of the CD177 gene made it possible to determine the risk of incidence of a healthcare-associated infection in a patient. This has never been shown or suggested in the literature. Patients having a high risk of contracting a care-associated infection could advantageously benefit from customized treatment.

Thus, a subject of the present invention is an in vitro or ex vivo method for determining the risk of incidence of a healthcare-associated infection, comprising a step of measuring the expression of CD177 in a biological sample from said patient.

In the context of the present invention:
The term “patient” denotes an individual (human) who has come into contact with a healthcare professional such as a doctor (for example a family physician) or a medical establishment or a healthcare facility (for example a hospital, and more particularly the emergency department, the resuscitation unit, an intensive care unit or an ongoing care unit, or a medicalized establishment for the elderly, such as an assisted living facility). The patient may for example be an elderly person, in the context of a vaccination protocol (particularly in an assisted living facility or else at a family physician);
An infection is said to be “healthcare-associated” if it occurs during, or after, a treatment (diagnostic, therapeutic, palliative, preventative, educational or operating) of a patient by a healthcare professional and if it was not present at the start of treatment. Healthcare-associated infections (HCAI) include infections contracted within a healthcare facility (referred to as nosocomial infections) but also those contracted during care administered outside this context. When the state of infection at the start of treatment is not known precisely, a period of at least 48 hours or a period longer than the incubation period is commonly accepted for defining an HCAI. For infections of the surgical site, infections are usually considered to be healthcare-associated when they occur in the 30 days following surgery or, if an implant, a prosthesis or a prosthetic material has been put in place, in the year following surgery. The infection may be of bacterial, fungal or viral origin. It may also relate to the reactivation of potentially pathogenic latent viruses, such as herpes viruses, for example CMV;
“Biological sample” relates to any sample originating from a patient, which may be of different natures, such as blood or derivatives thereof, sputum, urine, stools, skin, cerebrospinal fluid, bronchoalveolar lavage fluid, abdominal cavity puncture fluid, saliva, gastric secretions, sperm, seminal fluid, tears, spinal cord, trigeminal nerve ganglion, adipose tissue, lymphoid tissue, placental tissue. gastrointestinal tract tissue, genital tract tissue, central nervous system tissue. In particular, this sample may be a biological fluid, such as a blood sample or a blood-derived sample, which may particularly be chosen from total blood (as collected from a vein, i.e. containing white and red blood cells, platelets and plasma), plasma, serum, and any type(s) of cells extracted from the blood, such as peripheral blood mononuclear cells (PBMCs, containing lymphocytes (B, T and NK cells), dendritic cells and monocytes), subsets of B cells, purified monocytes, or neutrophils.

Preferably, in the method as described previously:
the patient is a patient within a healthcare facility, preferably within a hospital, more preferably still within the emergency department, the resuscitation unit, an intensive care unit or an ongoing care unit; particularly preferably, the patient is a patient in a septic state (more particularly in septic shock), a patient suffering from burns (more particularly severe burns), a patient suffering from an injury (more particularly a severe injury), or a patient undergoing surgery (more particularly major surgery); and
the method makes it possible to determine the risk of incidence of a nosocomial infection in said patient.

In the case of a patient in a septic state (already infected with a first infection), the method according to the invention makes it possible to determine the risk of incidence of a secondary infection. “Patient in a septic state” (or patient suffering from sepsis) means a patient having at least one life-threatening organ failure caused by an inappropriate host response to infection. “Septic shock” means a sub-type of sepsis in which hypotension persists despite sufficient vascular filling.

Preferably, the method according to the invention as described previously in all the embodiments thereof makes it possible to determine the risk of incidence of a healthcare-associated infection in a patient:
in the 30 days starting from the day of the immuno-inflammatory attack, more preferably still in the 15 days starting from the day of the immuno-inflammatory attack (i.e. the injury for patients suffering from an injury, the burn for patients suffering from burns, the surgery for patients undergoing surgery, or the sepsis diagnosis for patients in a septic state), i.e. during the 1st, 2nd, 3rd, 4th, 5th, 6th, 7th, 8th, 9th, 10th, 11th, 12th, 13th, 14th or 15th day starting from the immuno-inflammatory attack (the 1st day corresponding here to the day on which the immuno-inflammatory attack took place); it being possible for the biological sample to have been taken in particular during the 1st, 2nd, 3rd, 4th, 5th, 6th, 7th, 8th, 9th, 10th, 11th, 12th, 13th, 14th or 15th day starting from the immuno-inflammatory attack, preferably during the 1st, 2nd, 3rd, 4th, 5th, 6th or 7th day starting from the immuno-inflammatory attack, more preferably still during the 3rd, 4th, 5th, 6th or 7th day starting from the immuno-inflammatory attack; and/or
in the 7 days, 6 days, 5 days, or 4 days following the day on which the biological sample was taken (regardless of the day on which this sample was taken), i.e. during the 1st, 2nd, 3rd, 4th, 5th, 6th, or 7th day following the day on which the biological sample was taken (the 1st day corresponding here to the day after the day on which the biological sample was taken).

Preferably, the method as described previously, in all the embodiments thereof, additionally comprises a step of measuring, in the biological sample from the patient, the expression:
of at least one gene selected from the family of genes encoding molecules involved in the innate immune system, and/or
of at least one gene selected from the family of genes encoding molecules of the cell cycle, and/or
of at least one gene selected from the family of genes encoding cytokines, and/or
of at least one gene selected from the family of genes encoding anti-inflammatory cytokines, and/or
of at least one gene selected from the family of genes encoding pro-inflammatory cytokine receptors, located on chromosome 2 in the the region 2q11-2q12, and/or
of at least one gene selected from the family of genes encoding pro-inflammatory cytokines, and/or
of at least one gene selected from the family of genes encoding molecules involved in cytoskeleton formation, and/or
of at least one gene selected from the family of genes encoding molecules involved in gene expression and/or transcription, and/or
of at least one gene selected from the family of genes encoding growth factors, and/or
of at least one gene selected from the family of genes encoding molecules of the metabolism, and/or
of at least one gene selected from the family of genes encoding molecules involved in the adaptive immune system, and/or
of at least one gene selected from the family of genes encoding molecules involved in signal transduction, and/or
of at least one gene selected from the family of genes encoding molecules involved in modulation of the acute phase.

As examples of genes encoding molecules involved in the innate immune system, mention may be made of the following genes: GNLY, S100A9, C3AR1, ADGRE3, CX3CR1, IFIH1, OAS2, OAS3.

As an example of a gene encoding molecules of the cell cycle, mention may be made of the gene CCNB1IP1.

As examples of genes encoding cytokines, mention may be made of the following genes: IL15, IL2, MCP1(CCL2), CXCL10.

As examples of genes encoding anti-inflammatory cytokines, mention may be made of the following genes: IL10, IL1RN.

As examples of genes encoding pro-inflammatory cytokine receptors, located on chromosome 2 in the the region 2q11-2q12, mention may be made of the following genes: IL18R1, IL1R2, URI_ and IL18RAP.

As examples of genes encoding pro-inflammatory cytokines, mention may be made of the following genes: IFNG, IL1B, IL17A, IL18, IL6, TNF.

As examples of genes encoding molecules involved in cytoskeleton formation, mention may be made of the following genes: ARL14EP, GSN.

As examples of genes encoding molecules involved in gene expression and/or transcription, mention may be made of the following genes: CIITA, DYRK2, GATA3, MDC1, NFKB1, RORC, STAT4, TBX21, TDRD9.

As an example of a gene encoding growth factors, mention may be made of the gene CSF2.

As examples of genes encoding molecules of the metabolism, mention may be made of the following genes: ALOX5, BPGM, TRAP1.

As examples of genes encoding molecules involved in the adaptive immune system, mention may be made of the following genes: CD40LG, CD3D, BTLA, CD274, CTLA4, ICOS, PDCD1, TNFSF4, CD74, FCGR1A, LILRB2, TAP2.

As examples of genes encoding molecules involved in signal transduction, mention may be made of the following genes: FLT1, HAVCR2, IL7R, ZAP70.

As an example of a gene encoding molecules involved in modulation of the acute phase, mention may be made of the gene HP.

TABLE 1

| Biomarker (gene) | Chromosomal location (GRCh38/hg38) |
|---|---|
| CD177 | chr19: 43,353,659-43,363,172 |
| ADGRE3 | chr19: 14,619,117-14,690,027 |
| ALOX5 | chr10: 45,374,176-45,446,121 |
| ARL14EP | chr11: 30,323,099-30,338,458 |
| BPGM | chr7: 134,646,779-134,679,816 |
| BTLA | chr3: 112,463,966-112,499,702 |
| C3AR1 | chr12: 8,058,302-8,066,471 |
| CCNB1IP1 | chr14: 20,311,368-20,333,312 |
| CD40LG | chrX: 136,648,177-136,660,390 |
| CD274 | chr9: 5,450,381-5,470,567 |
| CD3D | chr11: 118,338,954-118,342,744 |
| CD74 | chr5: 150,400,041-150,412,936 |
| CIITA | chr16: 10,866,212-10,943,021 |
| CSF2 | chr5: 132,073,789-132,076,170 |
| CTLA4 | chr2: 203,867,771-203,873,965 |
| CX3CR1 | chr3: 39,263,494-39,281,735 |
| CXCL10 | chr4: 76,021,116-76,023,536 |
| DYRK2 | chr12: 67,648,338-67,665,406 |
| FCGR1A | chr1: 149,781,275-149,792,518 |
| FLT1 | chr13: 28,300,346-28,495,145 |
| GATA3 | chr10: 8,045,378-8,075,201 |
| GNLY | chr2: 85,685,175-85,698,854 |
| GSN | chr9: 121,207,794-121,332,844 |
| HAVCR2 | chr5: 157,085,832-157,142,869 |
| HP | chr16: 72,054,592-72,061,056 |
| ICOS | chr2: 203,936,731-203,961,579 |
| IFIH1 | chr2: 162,267,074-162,318,764 |
| IFNG | chr12: 68,154,768-68,159,741 |
| IL10 | chr1: 206,767,602-206,774,607 |
| IL1B | chr2: 112,829,751-112,836,903 |
| IL15 | chr4: 141,636,583-141,733,987 |
| IL1RN | chr2: 113,099,365-113,134,016 |
| IL1R1 | chr2: 102,064,544-102,179,874 |
| IL1R2 | chr2: 101,991,816-102,028,544 |
| IL17A | chr6: 52,186,375-52,190,638 |
| IL18 | chr11: 112,143,251-112,164,117 |
| IL18RAP | chr2: 102,418,558-102,452,568 |
| IL18R1 | chr2: 102,311,529-102,398,777 |
| IL2 | chr4: 122,451,470-122,456,725 |
| IL6 | chr7: 22,725,442-22,732,002 |
| IL7R | chr5: 35,852,695-35,879,603 |
| LILRB2 | chr19: 54,273,812-54,281,184 |
| MDCl | chr6: 30,699,807-30,717,966 |
| MCP1 (CCL2) | chr17: 34,255,218-34,257,203 |
| NFKB1 | chr4: 102,501,329-102,617,302 |
| OAS2 | chr12: 112,970,819-113,011,723 |
| OAS3 | chr12: 112,938,433-112,973,251 |
| PDCD1 | chr2: 241,849,881-241,858,908 |
| RORC | chr1: 151,806,071-151,832,238 |
| S100A9 | chr1: 153,357,854-153,361,027 |
| STAT4 | chr2: 191,029,576-191,246,172 |
| TAP2 | chr6: 32,821,833-32,838,823 |
| TBX21 | chr17: 47,733,236-47,746,122 |
| TDRD9 | chr14: 103,928,438-104,052,667 |
| TNF | chr6: 31,575,565-31,578,336 |
| TNFSF4 | chr1: 173,183,731-173,208,072 |
| TRAP1 | chr16: 3,651,639-3,717,597 |
| ZAP70 | chr2: 97,713,560-97,744,327 |

Table 1 Chromosomal location of the CD177 gene and other genes for which the expression can be measured in combination with that of CD177

TABLE 2

| | | Expression site | | |
|---|---|---|---|---|
| Families | Biomarkers | (type of blood cell) | Location | Immune function |
| Family of molecules involved | GNLY | gamma delta (γδ) T | Intracellular, secreted | Alarmin/defensin |
| in the innate immune system | | lymphocytes, CD4 and | (different isoforms) | |
| | | CD8 T lymphocytes | | |
| | S100A9 | Neutrophils, classical | Intracellular, secreted | Alarmin/defensin |
| | | monocytes | (different isoforms) | |
| | C3AR1 | Basophils, monocytes | Membrane | Complement cascade |
| | ADGRE3 | Basophils, monocytes | Membrane, secreted | Neutrophil |
| | | | (different isoforms) | migration/activation/degranulation |
| | CX3CR1 | Monocytes, NK | Membrane | Neutrophil |
| | | lymphocytes, CD4 and | | migration/activation/degranulation |
| | | CD8 T lymphocytes | | |
| | IFIH1 | Any cell type (neutrophils) | Intracellular | Interferon pathway |
| | OAS2 | Any cell type (monocytes, | Intracellular | Interferon pathway |
| | | plasmablasts) | | |
| | OAS3 | Neutrophils, myeloid | Intracellular | Interferon pathway |
| | | dendritic cells, monocytes | | |
| Family of molecules of the cell | CCNB1IP1 | Any cell type (monocytes, | Intracellular | Cell cycle |
| cycle | | neutrophils, basophils) | | |
| Family of cytokines | IL15 | Monocytes, SM B | Intracellular, secreted | T cell activation/proliferation |
| | | lymphocytes, MAIT | (different isoforms) | |
| | | lymphocytes | | |
| | IL2 | Th2 and Th17 helper T | Secreted | T cell activation/proliferation |
| | | lymphocytes, CD8 T | | |
| | | lymphocytes, central | | |
| | | memory (CM) T | | |
| | | lymphocytes | | |
| | MCP1 (CCL2) | Monocytes, naive B | Secreted | Monocyte recruitment |
| | | lymphocytes | | |
| | CXCL10 | Monocytes | Secreted | Signal transduction |
| Family of anti-inflammatory | IL10 | Myeloid dendritic cells, | Membrane, secreted | Anti-inflammatory cytokine |
| cytokines | | monocytes, follicular | (different isoforms) | |
| | | helper T lymphocytes, Th1 | | |
| | | helper T lymphocytes, | | |
| | | regulatory T lymphocytes, | | |
| | | gamma delta (γδ) T | | |
| | | lymphocytes | | |
| | IL1RN | Neutrophils, monocytes | Membrane, secreted | Anti-inflammatory cytokine |
| | | | (different isoforms) | |
| Family of pro-inflammatory | IL1R2 | Neutrophils | Membrane, secreted | Pro-inflammatory cytokine |
| cytokine receptors | | | (different isoforms) | receptor |
| | IL18RAP | Neutrophils, NK | Intracellular, secreted | Pro-inflammatory cytokine |
| | | lymphocytes, CD4 T | (different isoforms) | receptor |
| | | lymphocytes, MAIT | | |
| | | lymphocytes, gamma | | |
| | | delta (γδ) T lymphocytes | | |
| | IL18R1 | Myeloid dendritic cells, | Intracellular, secreted | Pro-inflammatory cytokine |
| | | plasmacytoid dendritic cells, | (different isoforms) | receptor |
| | | basophils, neutrophils, | | |
| | | T lymphocytes, | | |
| | | NK lymphocytes | | |
| | IL1R1 | Neutrophil, CD4 memory T | Membrane, secreted | Pro-inflammatory cytokine |
| | | lymphocytes, memory | (different isoforms) | receptor |
| | | regulatory T lymphocytes | | |
| Family of pro-inflammatory | IFNG | Gamma delta (γδ) T | Secreted | Interferon pathway |
| cytokines | | lymphocytes, CD4 and | | |
| | | CD8 T lymphocytes, NK | | |
| | | lymphocytes | | |
| | IL1B | Myeloid dendritic cells, | Intracellular, secreted | Pro-inflammatory cytokine |
| | | monocytes, neutrophils | (different isoforms) | |
| | IL17A | Follicular helper T | Secreted | Pro-inflammatory cytokine |
| | | lymphocytes, Th17 helper | | |
| | | T lymphocytes, regulatory | | |
| | | T lymphocytes | | |
| | IL18 | Myeloid dendritic cells, | Intracellular, secreted | Pro-inflammatory cytokine |
| | | monocytes, progenitor | (different isoforms) | |
| | | cells, plasmacytoid | | |
| | | dendritic cells, basophils, | | |
| | | neutrophils | | |
| | IL6 | Monocytes, naive B | Intracellular, secreted | Pro-inflammatory cytokine |
| | | lymphocytes | (different isoforms) | |
| | TNF | Monocytes | Intracellular, secreted | Pro-inflammatory cytokine |
| | | | (different isoforms) | |
| Family of molecules involved | ARL14EP | Any cell type except for | Intracellular | Class II major histocompatibility |
| in cytoskeleton formation | | neutrophils | | complex interaction/export |
| | GSN | Any cell type (neutrophils, | Intracellular, secreted | Actin filament regulation |
| | | B lymphocytes) | (different isoforms) | |
| Family of molecules involved | CIITA | Myeloid dendritic cells, | Intracellular | Antigen presentation |
| in gene expression and/or | | monocytes, B lymphocytes | | |
| transcription | DYRK2 | Any cell type (T lymphocytes) | Intracellular, secreted | Cell growth |
| | | | (different isoforms) | |
| | GATA3 | Th2 helper T lymphocytes, | Intracellular | T cell activation/proliferation |
| | | regulatory T lymphocytes | | |
| | MDC1 | Any cell type except for | Intracellular | DNA repair |
| | | neutrophils | | |
| | NFKB1 | Any cell type (myeloid | Intracellular | Gene expression/transcription |
| | | dendritic cells, monocytes, | | |
| | | neutrophils) | | |
| | RORC | MAIT lymphocytes, Th17 | Intracellular | T cell activation/proliferation |
| | | helper T lymphocytes | | |
| | STAT4 | T lymphocytes, NK | Intracellular | T cell activation/proliferation |
| | | lymphocytes | | |
| | TBX21 | NK lymphocytes, CD4 and | Intracellular | T cell activation/proliferation |
| | | CD8 T lymphocytes, MAIT | | |
| | | lymphocytes, gamma | | |
| | | delta (γδ) T lymphocytes | | |
| | TDRD9 | Basophils, classical | Intracellular | Gene expression/transcription |
| | | monocytes | | |
| Family of growth factors | CSF2 | NK lymphocytes, myeloid | Secreted | Cell growth |
| | | cells, lymphoid cells | | |
| Family of molecules of the | ALOX5 | Basophils, neutrophils, | Intracellular | Fatty acid metabolism |
| metabolism | | monocytes, B lymphocytes | | |
| | BPGM | Any cell type | Intracellular | Carbohydrate metabolism |
| | TRAP1 | Any cell type | Intracellular | Mitochondrial respiration |
| Family of molecules involved | CD40LG | T lymphocytes (except | Membrane, secreted | T cell activation/proliferation |
| in the adaptive immune | | CD8 T lymphocytes) | (different isoforms) | |
| system | CD3D | T lymphocytes | Intracellular, secreted | T cell activation/proliferation |
| | | | (different isoforms) | |
| | BTLA | B lymphocytes, naive B | Intracellular, secreted | Co-stimulation by CD28 |
| | | lymphocytes | (different isoforms) | family/checkpoint inhibitor |
| | CD274 | Basophils, neutrophils, T | Membrane | Co-stimulation by CD28 |
| | | lymphocytes | | family/checkpoint inhibitor |
| | CTLA4 | Regulatory T lymphocytes, | Intracellular, secreted | Co-stimulation by CD28 |
| | | CD4 T lymphocytes | (different isoforms) | family/checkpoint inhibitor |
| | ICOS | T lymphocytes | Membrane | Co-stimulation by CD28 |
| | | | | family/checkpoint inhibitor |
| | PDCD1 | CD8 EM T lymphocytes, T | Intracellular, secreted | Co-stimulation by CD28 |
| | | lymphocytes | (different isoforms) | family/checkpoint inhibitor |
| | TNFSF4 | Progenitors | Intracellular | Co-stimulation by CD28 |
| | | | | family/checkpoint inhibitor |
| | CD74 | Myeloid dendritic cells, | Membrane | Antigen presentation |
| | | monocytes, B lymphocytes, | | |
| | | plasmacytoid dendritic | | |
| | | cells | | |
| | FCGR1A | Neutrophils, monocytes | Membrane | Antigen presentation |
| | LILRB2 | Neutrophils, monocytes | Membrane | Antigen presentation |
| | TAP2 | Any cell type (neutrophils) | Membrane, secreted | Antigen presentation |
| Family of molecules involved | FLT1 | Neutrophils, plasmacytoid | Intracellular, membrane, | Tyrosine kinase receptor |
| in signal transduction | | dendritic cells | secreted (different | |
| | | | isoforms) | |
| | HAVCR2 | Myeloid dendritic cells, | Intracellular, secreted | Receptor |
| | | monocytes, NK | (different isoforms) | |
| | | lymphocytes | | |
| | IL7R | T lymphocytes | Intracellular, secreted | Cytokine receptor |
| | | | (different isoforms) | |
| | ZAP70 | NK lymphocytes, T | Intracellular | T cell activation/proliferation |
| | | lymphocytes | | |
| Family of molecules involved | HP | Monocytes, neutrophils, | Intracellular, secreted | Receptor (binding and absorption |
| in modulation of the acute | | basophils | (different isoforms) | of ligand by scavenger receptor/ |
| phase | | | | neutrophil degranulation) |

Table 2 Classification of biomarkers which can be used in combination with CD177, by families and according to their immune function. The site of expression (blood cell type) and the location are also indicated.

Also preferably, the method as described previously, in all the embodiments thereof, comprises, in addition to the step of measuring the expression of CD177, a step of measuring, in the biological sample from the patient, the expression:
of at least one, two, three, four, five, six, seven, eight gene(s) selected from the following genes: GNLY, S100A9, C3AR1, ADGRE3, CX3CR1, IFIH1, OAS2, OAS3, and/or
of the CCNB1IP1 gene, and/or
of at least one, two, three, four, gene(s) selected from the following genes: IL15, IL2, MCP1(CCL2), CXCL10, and/or
of at least one, two gene(s) selected from the following genes: IL10, IL1RN, and/or
of at least one, two, three, four, gene(s) selected from the following genes: IL18R1, IL1R2, IL1R1 and IL18RAP and/or
of at least one, two, three, four, five, six gene(s) selected from the following genes: IFNG, IL1B, IL17A, IL18, IL6, TNF, and/or
of at least one, two gene(s) selected from the following genes: ARL14EP, GSN, and/or
of at least one, two, three, four, five, six, seven, eight, nine gene(s) selected from the following genes: CIITA, DYRK2, GATA3, MDC1, NFKB1, RORC, STAT4, TBX21, TDRD9, and/or
of the CSF2 gene, and/or
of at least one, two, three, four, five gene(s) selected from the following genes: ALOX5, BPGM, TRAP1, and/or
of at least one, two, three, four, five, six, seven, eight, nine, ten, eleven, twelve gene(s) selected from the following genes: CD40LG, CD3D, BTLA, CD274, CTLA4, ICOS, PDCD1, TNFSF4, CD74, FCGR1A, LILRB2, TAP2, and/or
of at least one, two, three, four, gene(s) selected from the following genes: FLT1, HAVCR2, IL7R, ZAP70, and/or
of the HP gene.

More preferably still, the method as described previously, in all the embodiments thereof, comprises, in addition to the step of measuring the expression of CD177, a step of measuring, in the biological sample from the patient, the expression of at least one, two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six gene(s) selected from the following genes: GNLY, S100A9, C3AR1, ADGRE3, CX3CR1, OAS2, CCNB1IP1, IL10, IL1RN, IL1R2, IFNG, TNF, ARL14EP, CIITA, GATA3, MDC1, TDRD9, BPGM, CD3D, CD274, CTLA4, CD74, TAP2, IL7R, ZAP70, HP.

More preferably still, the method as described previously, in all the embodiments thereof, comprises, in addition to the step of measuring the expression of CD177, a step of measuring, in the biological sample from the patient, the expression of at least one, two, three, four, five, six, seven, eight, nine, ten, eleven gene(s) selected from the following genes: S100A9, C3AR1, CD177, CX3CR1, IL1R2, IFNG, CIITA, CD3D, CTLA4, CD74, TAP2, HP.

Particularly preferred combinations of one or more other gene(s), the expression of which can be measured in combination with the expression of CD177, are presented in table 3.

TABLE 3

| | |
|---|---|
| Combinations of | S100A9 |
| one gene | C3AR1 |
| | TAP2 |
| | CX3CR1 |
| | IL1R2 |
| | IFNG |
| | CIITA |
| | CD3D |
| | CTLA4 |
| | CD74 |
| | HP |
| Combinations of | S100A9, C3AR1 |
| two genes | S100A9, TAP2 |
| | S100A9, CX3CR1 |
| | S100A9, IL1R2 |
| | S100A9, IFNG |
| | S100A9, CIITA |
| | S100A9, CD3D |
| | S100A9, CTLA4 |
| | S100A9, CD74 |
| | S100A9, HP |
| | C3AR1, TAP2 |
| | C3AR1, CX3CR1 |
| | C3AR1, IL1R2 |
| | C3AR1, IFNG |
| | C3AR1, CIITA |
| | C3AR1, CD3D |
| | C3AR1, CTLA4 |
| | C3AR1, CD74 |
| | C3AR1, HP |
| | TAP2, CX3CR1 |
| | TAP2, IL1R2 |
| | TAP2, IFNG |
| | TAP2, CIITA |
| | TAP2, CD3D |
| | TAP2, CTLA4 |
| | TAP2, CD74 |
| | TAP2, HP |
| | CX3CR1, IL1R2 |
| | CX3CR1, IFNG |
| | CX3CR1, CIITA |
| | CX3CR1, CD3D |
| | CX3CR1, CTLA4 |
| | CX3CR1, CD74 |
| | CX3CR1, HP |
| | IL1R2, IFNG |
| | IL1R2, CIITA |
| | IL1R2, CD3D |
| | IL1R2, CTLA4 |
| | IL1R2, CD74 |
| | IL1R2, HP |
| | IFNG, CIITA |
| | IFNG, CD3D |
| | IFNG, CTLA4 |
| | IFNG, CD74 |
| | IFNG, HP |
| | CIITA, CD3D |
| | CIITA, CTLA4 |
| | CIITA, CD74 |
| | CIITA, HP |
| | CD3D, CTLA4 |
| | CD3D, CD74 |
| | CD3D, HP |
| | CTLA4, CD74 |
| | CTLA4, HP |
| | CD74, HP |
| Combinations of | S100A9, C3AR1, TAP2 |
| three genes | S100A9, C3AR1, CX3CR1 |
| | S100A9, C3AR1, IL1R2 |
| | S100A9, C3AR1, IFNG |
| | S100A9, C3AR1, CIITA |
| | S100A9, C3AR1, CD3D |
| | S100A9, C3AR1, CTLA4 |
| | S100A9, C3AR1, CD74 |
| | S100A9, C3AR1, HP |
| | S100A9, TAP2, CX3CR1 |
| | S100A9, TAP2, IL1R2 |
| | S100A9, TAP2, IFNG |
| | S100A9, TAP2, CIITA |
| | S100A9, TAP2, CD3D |
| | S100A9, TAP2, CTLA4 |
| | S100A9, TAP2, CD74 |
| | S100A9, TAP2, HP |
| | S100A9, CX3CR1, IL1R2 |
| | S100A9, CX3CR1, IFNG |
| | S100A9, CX3CR1, CIITA |
| | S100A9, CX3CR1, CD3D |
| | S100A9, CX3CR1, CTLA4 |
| | S100A9, CX3CR1, CD74 |
| | S100A9, CX3CR1, HP |
| | S100A9, IL1R2, IFNG |
| | S100A9, IL1R2, CIITA |
| | S100A9, IL1R2, CD3D |
| | S100A9, IL1R2, CTLA4 |
| | S100A9, IL1R2, CD74 |
| | S100A9, IL1R2, HP |
| | S100A9, IFNG, CIITA |
| | S100A9, IFNG, CD3D |
| | S100A9, IFNG, CTLA4 |
| | S100A9, IFNG, CD74 |
| | S100A9, IFNG, HP |
| | S100A9, CIITA, CD3D |
| | S100A9, CIITA, CTLA4 |
| | S100A9, CIITA, CD74 |
| | S100A9, CIITA, HP |
| | S100A9, CD3D, CTLA4 |
| | S100A9, CD3D, CD74 |
| | S100A9, CD3D, HP |
| | S100A9, CTLA4, CD74 |
| | S100A9, CTLA4, HP |
| | S100A9, CD74, HP |
| | C3AR1, TAP2, CX3CR1 |
| | C3AR1, TAP2, IL1R2 |
| | C3AR1, TAP2, IFNG |
| | C3AR1, TAP2, CIITA |
| | C3AR1, TAP2, CD3D |
| | C3AR1, TAP2, CTLA4 |
| | C3AR1, TAP2, CD74 |
| | C3AR1, TAP2, HP |
| | C3AR1, CX3CR1, IL1R2 |
| | C3AR1, CX3CR1, IFNG |
| | C3AR1, CX3CR1, CIITA |
| | C3AR1, CX3CR1, CD3D |
| | C3AR1, CX3CR1, CTLA4 |
| | C3AR1, CX3CR1, CD74 |
| | C3AR1, CX3CR1, HP |
| | C3AR1, IL1R2, IFNG |
| | C3AR1, IL1R2, CIITA |
| | C3AR1, IL1R2, CD3D |
| | C3AR1, IL1R2, CTLA4 |
| | C3AR1, IL1R2, CD74 |
| | C3AR1, IL1R2, HP |
| | C3AR1, IFNG, CIITA |
| | C3AR1, IFNG, CD3D |
| | C3AR1, IFNG, CTLA4 |
| | C3AR1, IFNG, CD74 |
| | C3AR1, IFNG, HP |
| | C3AR1, CIITA, CD3D |
| | C3AR1, CIITA, CTLA4 |
| | C3AR1, CIITA, CD74 |
| | C3AR1, CIITA, HP |
| | C3AR1, CD3D, CTLA4 |
| | C3AR1, CD3D, CD74 |
| | C3AR1, CD3D, HP |
| | C3AR1, CTLA4, CD74 |
| | C3AR1, CTLA4, HP |
| | C3AR1, CD74, HP |
| | TAP2, CX3CR1, IL1R2 |
| | TAP2, CX3CR1, IFNG |
| | TAP2, CX3CR1, CIITA |
| | TAP2, CX3CR1, CD3D |
| | TAP2, CX3CR1, CTLA4 |
| | TAP2, CX3CR1, CD74 |
| | TAP2, CX3CR1, HP |
| | TAP2, IL1R2, IFNG |
| | TAP2, IL1R2, CIITA |
| | TAP2, IL1R2, CD3D |
| | TAP2, IL1R2, CTLA4 |
| | TAP2, IL1R2, CD74 |
| | TAP2, IL1R2, HP |
| | TAP2, IFNG, CIITA |
| | TAP2, IFNG, CD3D |
| | TAP2, IFNG, CTLA4 |
| | TAP2, IFNG, CD74 |
| | TAP2, IFNG, HP |
| | TAP2, CIITA, CD3D |
| | TAP2, CIITA, CTLA4 |
| | TAP2, CIITA, CD74 |
| | TAP2, CIITA, HP |
| | TAP2, CD3D, CTLA4 |
| | TAP2, CD3D, CD74 |
| | TAP2, CD3D, HP |
| | TAP2, CTLA4, CD74 |
| | TAP2, CTLA4, HP |
| | TAP2, CD74, HP |
| | CX3CR1, IL1R2, IFNG |
| | CX3CR1, IL1R2, CIITA |
| | CX3CR1, IL1R2, CD3D |
| | CX3CR1, IL1R2, CTLA4 |
| | CX3CR1, IL1R2, CD74 |
| | CX3CR1, IL1R2, HP |
| | CX3CR1, IFNG, CIITA |
| | CX3CR1, IFNG, CD3D |
| | CX3CR1, IFNG, CTLA4 |
| | CX3CR1, IFNG, CD74 |
| | CX3CR1, IFNG, HP |
| | CX3CR1, CIITA, CD3D |
| | CX3CR1, CIITA, CTLA4 |
| | CX3CR1, CIITA, CD74 |
| | CX3CR1, CIITA, HP |
| | CX3CR1, CD3D, CTLA4 |
| | CX3CR1, CD3D, CD74 |
| | CX3CR1, CD3D, HP |
| | CX3CR1, CTLA4, CD74 |
| | CX3CR1, CTLA4, HP |
| | CX3CR1, CD74, HP |
| | IL1R2, IFNG, CIITA |
| | IL1R2, IFNG, CD3D |
| | IL1R2, IFNG, CTLA4 |
| | IL1R2, IFNG, CD74 |
| | IL1R2, IFNG, HP |
| | IL1R2, CIITA, CD3D |
| | IL1R2, CIITA, CTLA4 |
| | IL1R2, CIITA, CD74 |
| | IL1R2, CIITA, HP |
| | IL1R2, CD3D, CTLA4 |
| | IL1R2, CD3D, CD74 |
| | IL1R2, CD3D, HP |
| | IL1R2, CTLA4, CD74 |
| | IL1R2, CTLA4, HP |
| | IL1R2, CD74, HP |
| | IFNG, CIITA, CD3D |
| | IFNG, CIITA, CTLA4 |
| | IFNG, CIITA, CD74 |
| | IFNG, CIITA, HP |
| | IFNG, CD3D, CTLA4 |
| | IFNG, CD3D, CD74 |
| | IFNG, CD3D, HP |
| | IFNG, CTLA4, CD74 |
| | IFNG, CTLA4, HP |
| | IFNG, CD74, HP |
| | CIITA, CD3D, CTLA4 |
| | CIITA, CD3D, CD74 |
| | CIITA, CD3D, HP |
| | CIITA, CTLA4, CD74 |
| | CIITA, CTLA4, HP |
| | CIITA, CD74, HP |
| | CD3D, CTLA4, CD74 |
| | CD3D, CTLA4, HP |
| | CD3D, CD74, HP |
| | CTLA4, CD74, HP |
| Combinations of | S100A9, C3AR1, TAP2, CX3CR1 |
| four genes | S100A9, C3AR1, TAP2, IL1R2 |
| | S100A9, C3AR1, TAP2, IFNG |
| | S100A9, C3AR1, TAP2, CIITA |
| | S100A9, C3AR1, TAP2, CD3D |
| | S100A9, C3AR1, TAP2, CTLA4 |
| | S100A9, C3AR1, TAP2, CD74 |
| | S100A9, C3AR1, TAP2, HP |
| | S100A9, C3AR1, CX3CR1, IL1R2 |
| | S100A9, C3AR1, CX3CR1, IFNG |
| | S100A9, C3AR1, CX3CR1, CIITA |
| | S100A9, C3AR1, CX3CR1, CD3D |
| | S100A9, C3AR1, CX3CR1, CTLA4 |
| | S100A9, C3AR1, CX3CR1, CD74 |
| | S100A9, C3AR1, CX3CR1, HP |
| | S100A9, C3AR1, IL1R2, IFNG |
| | S100A9, C3AR1, IL1R2, CIITA |
| | S100A9, C3AR1, IL1R2, CD3D |
| | S100A9, C3AR1, IL1R2, CTLA4 |
| | S100A9, C3AR1, IL1R2, CD74 |
| | S100A9, C3AR1, IL1R2, HP |
| | S100A9, C3AR1, IFNG, CIITA |
| | S100A9, C3AR1, IFNG, CD3D |
| | S100A9, C3AR1, IFNG, CTLA4 |
| | S100A9, C3AR1, IFNG, CD74 |
| | S100A9, C3AR1, IFNG, HP |
| | S100A9, C3AR1, CIITA, CD3D |
| | S100A9, C3AR1, CIITA, CTLA4 |
| | S100A9, C3AR1, CIITA, CD74 |
| | S100A9, C3AR1, CIITA, HP |
| | S100A9, C3AR1, CD3D, CTLA4 |
| | S100A9, C3AR1, CD3D, CD74 |
| | S100A9, C3AR1, CD3D, HP |
| | S100A9, C3AR1, CTLA4, CD74 |
| | S100A9, C3AR1, CTLA4, HP |
| | S100A9, C3AR1, CD74, HP |
| | S100A9, TAP2, CX3CR1, IL1R2 |
| | S100A9, TAP2, CX3CR1, IFNG |
| | S100A9, TAP2, CX3CR1, CIITA |
| | S100A9, TAP2, CX3CR1, CD3D |
| | S100A9, TAP2, CX3CR1, CTLA4 |
| | S100A9, TAP2, CX3CR1, CD74 |
| | S100A9, TAP2, CX3CR1, HP |
| | S100A9, TAP2, IL1R2, IFNG |
| | S100A9, TAP2, IL1R2, CIITA |
| | S100A9, TAP2, IL1R2, CD3D |
| | S100A9, TAP2, IL1R2, CTLA4 |
| | S100A9, TAP2, IL1R2, CD74 |
| | S100A9, TAP2, IL1R2, HP |
| | S100A9, TAP2, IFNG, CIITA |
| | S100A9, TAP2, IFNG, CD3D |
| | S100A9, TAP2, IFNG, CTLA4 |
| | S100A9, TAP2, IFNG, CD74 |
| | S100A9, TAP2, IFNG, HP |
| | S100A9, TAP2, CIITA, CD3D |
| | S100A9, TAP2, CIITA, CTLA4 |
| | S100A9, TAP2, CIITA, CD74 |
| | S100A9, TAP2, CIITA, HP |
| | S100A9, TAP2, CD3D, CTLA4 |
| | S100A9, TAP2, CD3D, CD74 |
| | S100A9, TAP2, CD3D, HP |
| | S100A9, TAP2, CTLA4, CD74 |
| | S100A9, TAP2, CTLA4, HP |
| | S100A9, TAP2, CD74, HP |
| | S100A9, CX3CR1, IL1R2, IFNG |
| | S100A9, CX3CR1, IL1R2, CIITA |
| | S100A9, CX3CR1, IL1R2, CD3D |
| | S100A9, CX3CR1, IL1R2, CTLA4 |
| | S100A9, CX3CR1, IL1R2, CD74 |
| | S100A9, CX3CR1, IL1R2, HP |
| | S100A9, CX3CR1, IFNG, CIITA |
| | S100A9, CX3CR1, IFNG, CD3D |
| | S100A9, CX3CR1, IFNG, CTLA4 |
| | S100A9, CX3CR1, IFNG, CD74 |
| | S100A9, CX3CR1, IFNG, HP |
| | S100A9, CX3CR1, CIITA, CD3D |
| | S100A9, CX3CR1, CHTA, CTLA4 |
| | S100A9, CX3CR1, CHTA, CD74 |
| | S100A9, CX3CR1, CIITA, HP |
| | S100A9, CX3CR1, CD3D, CTLA4 |
| | S100A9, CX3CR1, CD3D, CD74 |
| | S100A9, CX3CR1, CD3D, HP |
| | S100A9, CX3CR1, CTLA4, CD74 |
| | S100A9, CX3CR1, CTLA4, HP |
| | S100A9, CX3CR1, CD74, HP |
| | S100A9, IL1R2, IFNG, CIITA |
| | S100A9, IL1R2, IFNG, CD3D |
| | S100A9, IL1R2, IFNG, CTLA4 |
| | S100A9, IL1R2, IFNG, CD74 |
| | S100A9, IL1R2, IFNG, HP |
| | S100A9, IL1R2, CIITA, CD3D |
| | S100A9, IL1R2, CIITA, CTLA4 |
| | S100A9, IL1R2, CIITA, CD74 |
| | S100A9, IL1R2, CIITA, HP |
| | S100A9, IL1R2, CD3D, CTLA4 |
| | S100A9, IL1R2, CD3D, CD74 |
| | S100A9, IL1R2, CD3D, HP |
| | S100A9, IL1R2, CTLA4, CD74 |
| | S100A9, IL1R2, CTLA4, HP |
| | S100A9, IL1R2, CD74, HP |
| | S100A9, IFNG, CIITA, CD3D |
| | S100A9, IFNG, CIITA, CTLA4 |
| | S100A9, IFNG, CIITA, CD74 |
| | S100A9, IFNG, CIITA, HP |
| | S100A9, IFNG, CD3D, CTLA4 |
| | S100A9, IFNG, CD3D, CD74 |
| | S100A9, IFNG, CD3D, HP |
| | S100A9, IFNG, CTLA4, CD74 |
| | S100A9, IFNG, CTLA4, HP |
| | S100A9, IFNG, CD74, HP |
| | S100A9, CIITA, CD3D, CTLA4 |
| | S100A9, CIITA, CD3D, CD74 |
| | S100A9, CIITA, CD3D, HP |
| | S100A9, CIITA, CTLA4, CD74 |
| | S100A9, CIITA, CTLA4, HP |
| | S100A9, CIITA, CD74, HP |
| | S100A9, CD3D, CTLA4, CD74 |
| | S100A9, CD3D, CTLA4, HP |
| | S100A9, CD3D, CD74, HP |
| | S100A9, CTLA4, CD74, HP |
| | C3AR1, TAP2, CX3CR1, IL1R2 |
| | C3AR1, TAP2, CX3CR1, IFNG |
| | C3AR1, TAP2, CX3CR1, CIITA |
| | C3AR1, TAP2, CX3CR1, CD3D |
| | C3AR1, TAP2, CX3CR1, CTLA4 |
| | C3AR1, TAP2, CX3CR1, CD74 |
| | C3AR1, TAP2, CX3CR1, HP |
| | C3AR1, TAP2, IL1R2, IFNG |
| | C3AR1, TAP2, IL1R2, CIITA |
| | C3AR1, TAP2, IL1R2, CD3D |
| | C3AR1, TAP2, IL1R2, CTLA4 |
| | C3AR1, TAP2, IL1R2, CD74 |
| | C3AR1, TAP2, IL1R2, HP |
| | C3AR1, TAP2, IFNG, CIITA |
| | C3AR1, TAP2, IFNG, CD3D |
| | C3AR1, TAP2, IFNG, CTLA4 |
| | C3AR1, TAP2, IFNG, CD74 |
| | C3AR1, TAP2, IFNG, HP |
| | C3AR1, TAP2, CIITA, CD3D |
| | C3AR1, TAP2, CIITA, CTLA4 |
| | C3AR1, TAP2, CIITA, CD74 |
| | C3AR1, TAP2, CIITA, HP |
| | C3AR1, TAP2, CD3D, CTLA4 |
| | C3AR1, TAP2, CD3D, CD74 |
| | C3AR1, TAP2, CD3D, HP |
| | C3AR1, TAP2, CTLA4, CD74 |
| | C3AR1, TAP2, CTLA4, HP |
| | C3AR1, TAP2, CD74, HP |
| | C3AR1, CX3CR1, IL1R2, IFNG |
| | C3AR1, CX3CR1, IL1R2, CIITA |
| | C3AR1, CX3CR1, IL1R2, CD3D |
| | C3AR1, CX3CR1, IL1R2, CTLA4 |
| | C3AR1, CX3CR1, IL1R2, CD74 |
| | C3AR1, CX3CR1, IL1R2, HP |
| | C3AR1, CX3CR1, IFNG, CIITA |
| | C3AR1, CX3CR1, IFNG, CD3D |
| | C3AR1, CX3CR1, IFNG, CTLA4 |
| | C3AR1, CX3CR1, IFNG, CD74 |
| | C3AR1, CX3CR1, IFNG, HP |
| | C3AR1, CX3CR1, CIITA, CD3D |
| | C3AR1, CX3CR1, CIITA, CTLA4 |
| | C3AR1, CX3CR1, CIITA, CD74 |
| | C3AR1, CX3CR1, CIITA, HP |
| | C3AR1, CX3CR1, CD3D, CTLA4 |
| | C3AR1, CX3CR1, CD3D, CD74 |
| | C3AR1, CX3CR1, CD3D, HP |
| | C3AR1, CX3CR1, CTLA4, CD74 |
| | C3AR1, CX3CR1, CTLA4, HP |
| | C3AR1, CX3CR1, CD74, HP |
| | C3AR1, IL1R2, IFNG, CIITA |
| | C3AR1, IL1R2, IFNG, CD3D |
| | C3AR1, IL1R2, IFNG, CTLA4 |
| | C3AR1, IL1R2, IFNG, CD74 |
| | C3AR1, IL1R2, IFNG, HP |
| | C3AR1, IL1R2, CIITA, CD3D |
| | C3AR1, IL1R2, CIITA, CTLA4 |
| | C3AR1, IL1R2, CIITA, CD74 |
| | C3AR1, IL1R2, CIITA, HP |
| | C3AR1, IL1R2, CD3D, CTLA4 |
| | C3AR1, IL1R2, CD3D, CD74 |
| | C3AR1, IL1R2, CD3D, HP |
| | C3AR1, IL1R2, CTLA4, CD74 |
| | C3AR1, IL1R2, CTLA4, HP |
| | C3AR1, IL1R2, CD74, HP |
| | C3AR1, IFNG, CIITA, CD3D |
| | C3AR1, IFNG, CIITA, CTLA4 |
| | C3AR1, IFNG, CIITA, CD74 |
| | C3AR1, IFNG, CIITA, HP |
| | C3AR1, IFNG, CD3D, CTLA4 |
| | C3AR1, IFNG, CD3D, CD74 |
| | C3AR1, IFNG, CD3D, HP |
| | C3AR1, IFNG, CTLA4, CD74 |
| | C3AR1, IFNG, CTLA4, HP |
| | C3AR1, IFNG, CD74, HP |
| | C3AR1, CIITA, CD3D, CTLA4 |
| | C3AR1, CIITA, CD3D, CD74 |
| | C3AR1, CIITA, CD3D, HP |
| | C3AR1, CIITA, CTLA4, CD74 |
| | C3AR1, CIITA, CTLA4, HP |
| | C3AR1, CIITA, CD74, HP |
| | C3AR1, CD3D, CTLA4, CD74 |
| | C3AR1, CD3D, CTLA4, HP |
| | C3AR1, CD3D, CD74, HP |
| | C3AR1, CTLA4, CD74, HP |
| | TAP2, CX3CR1, IL1R2, IFNG |
| | TAP2, CX3CR1, IL1R2, CIITA |
| | TAP2, CX3CR1, IL1R2, CD3D |
| | TAP2, CX3CR1, IL1R2, CTLA4 |
| | TAP2, CX3CR1, IL1R2, CD74 |
| | TAP2, CX3CR1, IL1R2, HP |
| | TAP2, CX3CR1, IFNG, CIITA |
| | TAP2, CX3CR1, IFNG, CD3D |
| | TAP2, CX3CR1, IFNG, CTLA4 |
| | TAP2, CX3CR1, IFNG, CD74 |
| | TAP2, CX3CR1, IFNG, HP |
| | TAP2, CX3CR1, CIITA, CD3D |
| | TAP2, CX3CR1, CIITA, CTLA4 |
| | TAP2, CX3CR1, CIITA, CD74 |
| | TAP2, CX3CR1, CIITA, HP |
| | TAP2, CX3CR1, CD3D, CTLA4 |
| | TAP2, CX3CR1, CD3D, CD74 |
| | TAP2, CX3CR1, CD3D, HP |
| | TAP2, CX3CR1, CTLA4, CD74 |
| | TAP2, CX3CR1, CTLA4, HP |
| | TAP2, CX3CR1, CD74, HP |
| | TAP2, IL1R2, IFNG, CIITA |
| | TAP2, IL1R2, IFNG, CD3D |
| | TAP2, IL1R2, IFNG, CTLA4 |
| | TAP2, IL1R2, IFNG, CD74 |
| | TAP2, IL1R2, IFNG, HP |
| | TAP2, IL1R2, CIITA, CD3D |
| | TAP2, IL1R2, CIITA, CTLA4 |
| | TAP2, IL1R2, CIITA, CD74 |
| | TAP2, IL1R2, CIITA, HP |
| | TAP2, IL1R2, CD3D, CTLA4 |
| | TAP2, IL1R2, CD3D, CD74 |
| | TAP2, IL1R2, CD3D, HP |
| | TAP2, IL1R2, CTLA4, CD74 |
| | TAP2, IL1R2, CTLA4, HP |
| | TAP2, IL1R2, CD74, HP |
| | TAP2, IFNG, CIITA, CD3D |
| | TAP2, IFNG, CIITA, CTLA4 |
| | TAP2, IFNG, CIITA, CD74 |
| | TAP2, IFNG, CIITA, HP |
| | TAP2, IFNG, CD3D, CTLA4 |
| | TAP2, IFNG, CD3D, CD74 |
| | TAP2, IFNG, CD3D, HP |
| | TAP2, IFNG, CTLA4, CD74 |
| | TAP2, IFNG, CTLA4, HP |
| | TAP2, IFNG, CD74, HP |
| | TAP2, CIITA, CD3D, CTLA4 |
| | TAP2, CIITA, CD3D, CD74 |
| | TAP2, CIITA, CD3D, HP |
| | TAP2, CIITA, CTLA4, CD74 |
| | TAP2, CIITA, CTLA4, HP |
| | TAP2, CIITA, CD74, HP |
| | TAP2, CD3D, CTLA4, CD74 |
| | TAP2, CD3D, CTLA4, HP |
| | TAP2, CD3D, CD74, HP |
| | TAP2, CTLA4, CD74, HP |
| | CX3CR1, IL1R2, IFNG, CIITA |
| | CX3CR1, IL1R2, IFNG, CD3D |
| | CX3CR1, IL1R2, IFNG, CTLA4 |
| | CX3CR1, IL1R2, IFNG, CD74 |
| | CX3CR1, IL1R2, IFNG, HP |
| | CX3CR1, IL1R2, CIITA, CD3D |
| | CX3CR1, IL1R2, CIITA, CTLA4 |
| | CX3CR1, IL1R2, CIITA, CD74 |
| | CX3CR1, IL1R2, CIITA, HP |
| | CX3CR1, IL1R2, CD3D, CTLA4 |
| | CX3CR1, IL1R2, CD3D, CD74 |
| | CX3CR1, IL1R2, CD3D, HP |
| | CX3CR1, IL1R2, CTLA4, CD74 |
| | CX3CR1, IL1R2, CTLA4, HP |
| | CX3CR1, IL1R2, CD74, HP |
| | CX3CR1, IFNG, CIITA, CD3D |
| | CX3CR1, IFNG, CIITA, CTLA4 |
| | CX3CR1, IFNG, CIITA, CD74 |
| | CX3CR1, IFNG, CIITA, HP |
| | CX3CR1, IFNG, CD3D, CTLA4 |
| | CX3CR1, IFNG, CD3D, CD74 |
| | CX3CR1, IFNG, CD3D, HP |
| | CX3CR1, IFNG, CTLA4, CD74 |
| | CX3CR1, IFNG, CTLA4, HP |
| | CX3CR1, IFNG, CD74, HP |
| | CX3CR1, CIITA, CD3D, CTLA4 |
| | CX3CR1, CIITA, CD3D, CD74 |
| | CX3CR1, CIITA, CD3D, HP |
| | CX3CR1, CIITA, CTLA4, CD74 |
| | CX3CR1, CIITA, CTLA4, HP |
| | CX3CR1, CIITA, CD74, HP |
| | CX3CR1, CD3D, CTLA4, CD74 |
| | CX3CR1, CD3D, CTLA4, HP |
| | CX3CR1, CD3D, CD74, HP |
| | CX3CR1, CTLA4, CD74, HP |
| | IL1R2, IFNG, CIITA, CD3D |
| | IL1R2, IFNG, CIITA, CTLA4 |
| | IL1R2, IFNG, CIITA, CD74 |
| | IL1R2, IFNG, CIITA, HP |
| | IL1R2, IFNG, CD3D, CTLA4 |
| | IL1R2, IFNG, CD3D, CD74 |
| | IL1R2, IFNG, CD3D, HP |
| | IL1R2, IFNG, CTLA4, CD74 |
| | IL1R2, IFNG, CTLA4, HP |
| | IL1R2, IFNG, CD74, HP |
| | IL1R2, CIITA, CD3D, CTLA4 |
| | IL1R2, CIITA, CD3D, CD74 |
| | IL1R2, CIITA, CD3D, HP |
| | IL1R2, CIITA, CTLA4, CD74 |
| | IL1R2, CIITA, CTLA4, HP |
| | IL1R2, CIITA, CD74, HP |
| | IL1R2, CD3D, CTLA4, CD74 |
| | IL1R2, CD3D, CTLA4, HP |
| | IL1R2, CD3D, CD74, HP |
| | IL1R2, CTLA4, CD74, HP |
| | IFNG, CIITA, CD3D, CTLA4 |
| | IFNG, CIITA, CD3D, CD74 |
| | IFNG, CIITA, CD3D, HP |
| | IFNG, CIITA, CTLA4, CD74 |
| | IFNG, CIITA, CTLA4, HP |
| | IFNG, CIITA, CD74, HP |
| | IFNG, CD3D, CTLA4, CD74 |
| | IFNG, CD3D, CTLA4, HP |
| | IFNG, CD3D, CD74, HP |
| | IFNG, CTLA4, CD74, HP |
| | CIITA, CD3D, CTLA4, CD74 |
| | CIITA, CD3D, CTLA4, HP |
| | CIITA, CD3D, CD74, HP |
| | CIITA, CTLA4, CD74, HP |
| | CD3D, CTLA4, CD74, HP |
| Combinations of | S100A9, C3AR1, TAP2, CX3CR1, IL1R2 |
| five genes | S100A9, C3AR1, TAP2, CX3CR1, IFNG |
| | S100A9, C3AR1, TAP2, CX3CR1, CIITA |
| | S100A9, C3AR1, TAP2, CX3CR1, CD3D |
| | S100A9, C3AR1, TAP2, CX3CR1, CTLA4 |
| | S100A9, C3AR1, TAP2, CX3CR1, CD74 |
| | S100A9, C3AR1, TAP2, CX3CR1, HP |
| | S100A9, C3AR1, TAP2, IL1R2, IFNG |
| | S100A9, C3AR1, TAP2, IL1R2, CIITA |
| | S100A9, C3AR1, TAP2, IL1R2, CD3D |
| | S100A9, C3AR1, TAP2, IL1R2, CTLA4 |
| | S100A9, C3AR1, TAP2, IL1R2, CD74 |
| | S100A9, C3AR1, TAP2, IL1R2, HP |
| | S100A9, C3AR1, TAP2, IFNG, CIITA |
| | S100A9, C3AR1, TAP2, IFNG, CD3D |
| | S100A9, C3AR1, TAP2, IFNG, CTLA4 |
| | S100A9, C3AR1, TAP2, IFNG, CD74 |
| | S100A9, C3AR1, TAP2, IFNG, HP |
| | S100A9, C3AR1, TAP2, CIITA, CD3D |
| | S100A9, C3AR1, TAP2, CIITA, CTLA4 |
| | S100A9, C3AR1, TAP2, CIITA, CD74 |
| | S100A9, C3AR1, TAP2, CIITA, HP |
| | S100A9, C3AR1, TAP2, CD3D, CTLA4 |
| | S100A9, C3AR1, TAP2, CD3D, CD74 |
| | S100A9, C3AR1, TAP2, CD3D, HP |
| | S100A9, C3AR1, TAP2, CTLA4, CD74 |
| | S100A9, C3AR1, TAP2, CTLA4, HP |
| | S100A9, C3AR1, TAP2, CD74, HP |
| | S100A9, C3AR1, CX3CR1, IL1R2, IFNG |
| | S100A9, C3AR1, CX3CR1, IL1R2, CIITA |
| | S100A9, C3AR1, CX3CR1, IL1R2, CD3D |
| | S100A9, C3AR1, CX3CR1, IL1R2, CTLA4 |
| | S100A9, C3AR1, CX3CR1, IL1R2, CD74 |
| | S100A9, C3AR1, CX3CR1, IL1R2, HP |
| | S100A9, C3AR1, CX3CR1, IFNG, CIITA |
| | S100A9, C3AR1, CX3CR1, IFNG, CD3D |
| | S100A9, C3AR1, CX3CR1, IFNG, CTLA4 |
| | S100A9, C3AR1, CX3CR1, IFNG, CD74 |
| | S100A9, C3AR1, CX3CR1, IFNG, HP |
| | S100A9, C3AR1, CX3CR1, CIITA, CD3D |
| | S100A9, C3AR1, CX3CR1, CIITA, CTLA4 |
| | S100A9, C3AR1, CX3CR1, CIITA, CD74 |
| | S100A9, C3AR1, CX3CR1, CIITA, HP |
| | S100A9, C3AR1, CX3CR1, CD3D, CTLA4 |
| | S100A9, C3AR1, CX3CR1, CD3D, CD74 |
| | S100A9, C3AR1, CX3CR1, CD3D, HP |
| | S100A9, C3AR1, CX3CR1, CTLA4, CD74 |
| | S100A9, C3AR1, CX3CR1, CTLA4, HP |
| | S100A9, C3AR1, CX3CR1, CD74, HP |
| | S100A9, C3AR1, IL1R2, IFNG, CIITA |
| | S100A9, C3AR1, IL1R2, IFNG, CD3D |
| | S100A9, C3AR1, IL1R2, IFNG, CTLA4 |
| | S100A9, C3AR1, IL1R2, IFNG, CD74 |
| | S100A9, C3AR1, IL1R2, IFNG, HP |
| | S100A9, C3AR1, IL1R2, CIITA, CD3D |
| | S100A9, C3AR1, IL1R2, CIITA, CTLA4 |
| | S100A9, C3AR1, IL1R2, CIITA, CD74 |
| | S100A9, C3AR1, IL1R2, CIITA, HP |
| | S100A9, C3AR1, IL1R2, CD3D, CTLA4 |
| | S100A9, C3AR1, IL1R2, CD3D, CD74 |
| | S100A9, C3AR1, IL1R2, CD3D, HP |
| | S100A9, C3AR1, IL1R2, CTLA4, CD74 |
| | S100A9, C3AR1, IL1R2, CTLA4, HP |
| | S100A9, C3AR1, IL1R2, CD74, HP |
| | S100A9, C3AR1, IFNG, CIITA, CD3D |
| | S100A9, C3AR1, IFNG, CIITA, CTLA4 |
| | S100A9, C3AR1, IFNG, CIITA, CD74 |
| | S100A9, C3AR1, IFNG, CIITA, HP |
| | S100A9, C3AR1, IFNG, CD3D, CTLA4 |
| | S100A9, C3AR1, IFNG, CD3D, CD74 |
| | S100A9, C3AR1, IFNG, CD3D, HP |
| | S100A9, C3AR1, IFNG, CTLA4, CD74 |
| | S100A9, C3AR1, IFNG, CTLA4, HP |
| | S100A9, C3AR1, IFNG, CD74, HP |
| | S100A9, C3AR1, CIITA, CD3D, CTLA4 |
| | S100A9, C3AR1, CIITA, CD3D, CD74 |
| | S100A9, C3AR1, CIITA, CD3D, HP |
| | S100A9, C3AR1, CIITA, CTLA4, CD74 |
| | S100A9, C3AR1, CIITA, CTLA4, HP |
| | S100A9, C3AR1, CIITA, CD74, HP |
| | S100A9, C3AR1, CD3D, CTLA4, CD74 |
| | S100A9, C3AR1, CD3D, CTLA4, HP |
| | S100A9, C3AR1, CD3D, CD74, HP |
| | S100A9, C3AR1, CTLA4, CD74, HP |
| | S100A9, TAP2, CX3CR1, IL1R2, IFNG |
| | S100A9, TAP2, CX3CR1, IL1R2, CIITA |
| | S100A9, TAP2, CX3CR1, IL1R2, CD3D |
| | S100A9, TAP2, CX3CR1, IL1R2, CTLA4 |
| | S100A9, TAP2, CX3CR1, IL1R2, CD74 |
| | S100A9, TAP2, CX3CR1, IL1R2, HP |
| | S100A9, TAP2, CX3CR1, IFNG, CIITA |
| | S100A9, TAP2, CX3CR1, IFNG, CD3D |
| | S100A9, TAP2, CX3CR1, IFNG, CTLA4 |
| | S100A9, TAP2, CX3CR1, IFNG, CD74 |
| | S100A9, TAP2, CX3CR1, IFNG, HP |
| | S100A9, TAP2, CX3CR1, CIITA, CD3D |
| | S100A9, TAP2, CX3CR1, CIITA, CTLA4 |
| | S100A9, TAP2, CX3CR1, CIITA, CD74 |
| | S100A9, TAP2, CX3CR1, CIITA, HP |
| | S100A9, TAP2, CX3CR1, CD3D, CTLA4 |
| | S100A9, TAP2, CX3CR1, CD3D, CD74 |
| | S100A9, TAP2, CX3CR1, CD3D, HP |
| | S100A9, TAP2, CX3CR1, CTLA4, CD74 |
| | S100A9, TAP2, CX3CR1, CTLA4, HP |
| | S100A9, TAP2, CX3CR1, CD74, HP |
| | S100A9, TAP2, IL1R2, IFNG, CIITA |
| | S100A9, TAP2, IL1R2, IFNG, CD3D |
| | S100A9, TAP2, IL1R2, IFNG, CTLA4 |
| | S100A9, TAP2, IL1R2, IFNG, CD74 |
| | S100A9, TAP2, IL1R2, IFNG, HP |
| | S100A9, TAP2, IL1R2, CIITA, CD3D |
| | S100A9, TAP2, IL1R2, CIITA, CTLA4 |
| | S100A9, TAP2, IL1R2, CIITA, CD74 |
| | S100A9, TAP2, IL1R2, CIITA, HP |
| | S100A9, TAP2, IL1R2, CD3D, CTLA4 |
| | S100A9, TAP2, IL1R2, CD3D, CD74 |
| | S100A9, TAP2, IL1R2, CD3D, HP |
| | S100A9, TAP2, IL1R2, CTLA4, CD74 |
| | S100A9, TAP2, IL1R2, CTLA4, HP |
| | S100A9, TAP2, IL1R2, CD74, HP |
| | S100A9, TAP2, IFNG, CIITA, CD3D |
| | S100A9, TAP2, IFNG, CIITA, CTLA4 |
| | S100A9, TAP2, IFNG, CIITA, CD74 |
| | S100A9, TAP2, IFNG, CIITA, HP |
| | S100A9, TAP2, IFNG, CD3D, CTLA4 |
| | S100A9, TAP2, IFNG, CD3D, CD74 |
| | S100A9, TAP2, IFNG, CD3D, HP |
| | S100A9, TAP2, IFNG, CTLA4, CD74 |
| | S100A9, TAP2, IFNG, CTLA4, HP |
| | S100A9, TAP2, IFNG, CD74, HP |
| | S100A9, TAP2, CIITA, CD3D, CTLA4 |
| | S100A9, TAP2, CIITA, CD3D, CD74 |
| | S100A9, TAP2, CIITA, CD3D, HP |
| | S100A9, TAP2, CIITA, CTLA4, CD74 |
| | S100A9, TAP2, CIITA, CTLA4, HP |
| | S100A9, TAP2, CIITA, CD74, HP |
| | S100A9, TAP2, CD3D, CTLA4, CD74 |
| | S100A9, TAP2, CD3D, CTLA4, HP |
| | S100A9, TAP2, CD3D, CD74, HP |
| | S100A9, TAP2, CTLA4, CD74, HP |
| | S100A9, CX3CR1, IL1R2, IFNG, CIITA |
| | S100A9, CX3CR1, IL1R2, IFNG, CD3D |
| | S100A9, CX3CR1, IL1R2, IFNG, CTLA4 |
| | S100A9, CX3CR1, IL1R2, IFNG, CD74 |
| | S100A9, CX3CR1, IL1R2, IFNG, HP |
| | S100A9, CX3CR1, IL1R2, CIITA, CD3D |
| | S100A9, CX3CR1, IL1R2, CIITA, CTLA4 |
| | S100A9, CX3CR1, IL1R2, CIITA, CD74 |
| | S100A9, CX3CR1, IL1R2, CIITA, HP |
| | S100A9, CX3CR1, IL1R2, CD3D, CTLA4 |
| | S100A9, CX3CR1, IL1R2, CD3D, CD74 |
| | S100A9, CX3CR1, IL1R2, CD3D, HP |
| | S100A9, CX3CR1, IL1R2, CTLA4, CD74 |
| | S100A9, CX3CR1, IL1R2, CTLA4, HP |
| | S100A9, CX3CR1, IL1R2, CD74, HP |
| | S100A9, CX3CR1, IFNG, CIITA, CD3D |
| | S100A9, CX3CR1, IFNG, CIITA, CTLA4 |
| | S100A9, CX3CR1, IFNG, CIITA, CD74 |
| | S100A9, CX3CR1, IFNG, CIITA, HP |
| | S100A9, CX3CR1, IFNG, CD3D, CTLA4 |
| | S100A9, CX3CR1, IFNG, CD3D, CD74 |
| | S100A9, CX3CR1, IFNG, CD3D, HP |
| | S100A9, CX3CR1, IFNG, CTLA4, CD74 |
| | S100A9, CX3CR1, IFNG, CTLA4, HP |
| | S100A9, CX3CR1, IFNG, CD74, HP |
| | S100A9, CX3CR1, CIITA, CD3D, CTLA4 |
| | S100A9, CX3CR1, CIITA, CD3D, CD74 |
| | S100A9, CX3CR1, CIITA, CD3D, HP |
| | S100A9, CX3CR1, CIITA, CTLA4, CD74 |
| | S100A9, CX3CR1, CIITA, CTLA4, HP |
| | S100A9, CX3CR1, CIITA, CD74, HP |
| | S100A9, CX3CR1, CD3D, CTLA4, CD74 |
| | S100A9, CX3CR1, CD3D, CTLA4, HP |
| | S100A9, CX3CR1, CD3D, CD74, HP |
| | S100A9, CX3CR1, CTLA4, CD74, HP |
| | S100A9, IL1R2, IFNG, CIITA, CD3D |
| | S100A9, IL1R2, IFNG, CIITA, CTLA4 |
| | S100A9, IL1R2, IFNG, CIITA, CD74 |
| | S100A9, IL1R2, IFNG, CIITA, HP |
| | S100A9, IL1R2, IFNG, CD3D, CTLA4 |
| | S100A9, IL1R2, IFNG, CD3D, CD74 |
| | S100A9, IL1R2, IFNG, CD3D, HP |
| | S100A9, IL1R2, IFNG, CTLA4, CD74 |
| | S100A9, IL1R2, IFNG, CTLA4, HP |
| | S100A9, IL1R2, IFNG, CD74, HP |
| | S100A9, IL1R2, CIITA, CD3D, CTLA4 |
| | S100A9, IL1R2, CIITA, CD3D, CD74 |
| | S100A9, IL1R2, CIITA, CD3D, HP |
| | S100A9, IL1R2, CIITA, CTLA4, CD74 |
| | S100A9, IL1R2, CIITA, CTLA4, HP |
| | S100A9, IL1R2, CIITA, CD74, HP |
| | S100A9, IL1R2, CD3D, CTLA4, CD74 |
| | S100A9, IL1R2, CD3D, CTLA4, HP |
| | S100A9, IL1R2, CD3D, CD74, HP |
| | S100A9, IL1R2, CTLA4, CD74, HP |
| | S100A9, IFNG, CIITA, CD3D, CTLA4 |
| | S100A9, IFNG, CIITA, CD3D, CD74 |
| | S100A9, IFNG, CIITA, CD3D, HP |
| | S100A9, IFNG, CIITA, CTLA4, CD74 |
| | S100A9, IFNG, CIITA, CTLA4, HP |
| | S100A9, IFNG, CIITA, CD74, HP |
| | S100A9, IFNG, CD3D, CTLA4, CD74 |
| | S100A9, IFNG, CD3D, CTLA4, HP |
| | S100A9, IFNG, CD3D, CD74, HP |
| | S100A9, IFNG, CTLA4, CD74, HP |
| | S100A9, CIITA, CD3D, CTLA4, CD74 |
| | S100A9, CIITA, CD3D, CTLA4, HP |
| | S100A9, CIITA, CD3D, CD74, HP |
| | S100A9, CIITA, CTLA4, CD74, HP |
| | S100A9, CD3D, CTLA4, CD74, HP |
| | C3AR1, TAP2, CX3CR1, IL1R2, IFNG |
| | C3AR1, TAP2, CX3CR1, IL1R2, CIITA |
| | C3AR1, TAP2, CX3CR1, IL1R2, CD3D |
| | C3AR1, TAP2, CX3CR1, IL1R2, CTLA4 |
| | C3AR1, TAP2, CX3CR1, IL1R2, CD74 |
| | C3AR1, TAP2, CX3CR1, IL1R2, HP |
| | C3AR1, TAP2, CX3CR1, IFNG, CIITA |
| | C3AR1, TAP2, CX3CR1, IFNG, CD3D |
| | C3AR1, TAP2, CX3CR1, IFNG, CTLA4 |
| | C3AR1, TAP2, CX3CR1, IFNG, CD74 |
| | C3AR1, TAP2, CX3CR1, IFNG, HP |
| | C3AR1, TAP2, CX3CR1, CIITA, CD3D |
| | C3AR1, TAP2, CX3CR1, CIITA, CTLA4 |
| | C3AR1, TAP2, CX3CR1, CIITA, CD74 |
| | C3AR1, TAP2, CX3CR1, CIITA, HP |
| | C3AR1, TAP2, CX3CR1, CD3D, CTLA4 |
| | C3AR1, TAP2, CX3CR1, CD3D, CD74 |
| | C3AR1, TAP2, CX3CR1, CD3D, HP |
| | C3AR1, TAP2, CX3CR1, CTLA4, CD74 |
| | C3AR1, TAP2, CX3CR1, CTLA4, HP |
| | C3AR1, TAP2, CX3CR1, CD74, HP |
| | C3AR1, TAP2, IL1R2, IFNG, CIITA |
| | C3AR1, TAP2, IL1R2, IFNG, CD3D |
| | C3AR1, TAP2, IL1R2, IFNG, CTLA4 |
| | C3AR1, TAP2, IL1R2, IFNG, CD74 |
| | C3AR1, TAP2, IL1R2, IFNG, HP |
| | C3AR1, TAP2, IL1R2, CIITA, CD3D |
| | C3AR1, TAP2, IL1R2, CIITA, CTLA4 |
| | C3AR1, TAP2, IL1R2, CIITA, CD74 |
| | C3AR1, TAP2, IL1R2, CIITA, HP |
| | C3AR1, TAP2, IL1R2, CD3D, CTLA4 |
| | C3AR1, TAP2, IL1R2, CD3D, CD74 |
| | C3AR1, TAP2, IL1R2, CD3D, HP |
| | C3AR1, TAP2, IL1R2, CTLA4, CD74 |
| | C3AR1, TAP2, IL1R2, CTLA4, HP |
| | C3AR1, TAP2, IL1R2, CD74, HP |
| | C3AR1, TAP2, IFNG, CIITA, CD3D |
| | C3AR1, TAP2, IFNG, CIITA, CTLA4 |
| | C3AR1, TAP2, IFNG, CIITA, CD74 |
| | C3AR1, TAP2, IFNG, CIITA, HP |
| | C3AR1, TAP2, IFNG, CD3D, CTLA4 |
| | C3AR1, TAP2, IFNG, CD3D, CD74 |
| | C3AR1, TAP2, IFNG, CD3D, HP |
| | C3AR1, TAP2, IFNG, CTLA4, CD74 |
| | C3AR1, TAP2, IFNG, CTLA4, HP |
| | C3AR1, TAP2, IFNG, CD74, HP |
| | C3AR1, TAP2, CIITA, CD3D, CTLA4 |
| | C3AR1, TAP2, CIITA, CD3D, CD74 |
| | C3AR1, TAP2, CIITA, CD3D, HP |
| | C3AR1, TAP2, CIITA, CTLA4, CD74 |
| | C3AR1, TAP2, CIITA, CTLA4, HP |
| | C3AR1, TAP2, CIITA, CD74, HP |
| | C3AR1, TAP2, CD3D, CTLA4, CD74 |
| | C3AR1, TAP2, CD3D, CTLA4, HP |
| | C3AR1, TAP2, CD3D, CD74, HP |
| | C3AR1, TAP2, CTLA4, CD74, HP |
| | C3AR1, CX3CR1, IL1R2, IFNG, CIITA |
| | C3AR1, CX3CR1, IL1R2, IFNG, CD3D |
| | C3AR1, CX3CR1, IL1R2, IFNG, CTLA4 |
| | C3AR1, CX3CR1, IL1R2, IFNG, CD74 |
| | C3AR1, CX3CR1, IL1R2, IFNG, HP |
| | C3AR1, CX3CR1, IL1R2, CIITA, CD3D |
| | C3AR1, CX3CR1, IL1R2, CIITA, CTLA4 |
| | C3AR1, CX3CR1, IL1R2, CIITA, CD74 |
| | C3AR1, CX3CR1, IL1R2, CIITA, HP |
| | C3AR1, CX3CR1, IL1R2, CD3D, CTLA4 |
| | C3AR1, CX3CR1, IL1R2, CD3D, CD74 |
| | C3AR1, CX3CR1, IL1R2, CD3D, HP |
| | C3AR1, CX3CR1, IL1R2, CTLA4, CD74 |
| | C3AR1, CX3CR1, IL1R2, CTLA4, HP |
| | C3AR1, CX3CR1, IL1R2, CD74, HP |
| | C3AR1, CX3CR1, IFNG, CIITA, CD3D |
| | C3AR1, CX3CR1, IFNG, CIITA, CTLA4 |
| | C3AR1, CX3CR1, IFNG, CIITA, CD74 |
| | C3AR1, CX3CR1, IFNG, CIITA, HP |
| | C3AR1, CX3CR1, IFNG, CD3D, CTLA4 |
| | C3AR1, CX3CR1, IFNG, CD3D, CD74 |
| | C3AR1, CX3CR1, IFNG, CD3D, HP |
| | C3AR1, CX3CR1, IFNG, CTLA4, CD74 |
| | C3AR1, CX3CR1, IFNG, CTLA4, HP |
| | C3AR1, CX3CR1, IFNG, CD74, HP |
| | C3AR1, CX3CR1, CIITA, CD3D, CTLA4 |
| | C3AR1, CX3CR1, CIITA, CD3D, CD74 |
| | C3AR1, CX3CR1, CIITA, CD3D, HP |
| | C3AR1, CX3CR1, CIITA, CTLA4, CD74 |
| | C3AR1, CX3CR1, CIITA, CTLA4, HP |
| | C3AR1, CX3CR1, CIITA, CD74, HP |
| | C3AR1, CX3CR1, CD3D, CTLA4, CD74 |
| | C3AR1, CX3CR1, CD3D, CTLA4, HP |
| | C3AR1, CX3CR1, CD3D, CD74, HP |
| | C3AR1, CX3CR1, CTLA4, CD74, HP |
| | C3AR1, IL1R2, IFNG, CIITA, CD3D |
| | C3AR1, IL1R2, IFNG, CIITA, CTLA4 |
| | C3AR1, IL1R2, IFNG, CIITA, CD74 |
| | C3AR1, IL1R2, IFNG, CIITA, HP |
| | C3AR1, IL1R2, IFNG, CD3D, CTLA4 |
| | C3AR1, IL1R2, IFNG, CD3D, CD74 |
| | C3AR1, IL1R2, IFNG, CD3D, HP |
| | C3AR1, IL1R2, IFNG, CTLA4, CD74 |
| | C3AR1, IL1R2, IFNG, CTLA4, HP |
| | C3AR1, IL1R2, IFNG, CD74, HP |
| | C3AR1, IL1R2, CIITA, CD3D, CTLA4 |
| | C3AR1, IL1R2, CIITA, CD3D, CD74 |
| | C3AR1, IL1R2, CIITA, CD3D, HP |
| | C3AR1, IL1R2, CIITA, CTLA4, CD74 |
| | C3AR1, IL1R2, CIITA, CTLA4, HP |
| | C3AR1, IL1R2, CIITA, CD74, HP |
| | C3AR1, IL1R2, CD3D, CTLA4, CD74 |
| | C3AR1, IL1R2, CD3D, CTLA4, HP |
| | C3AR1, IL1R2, CD3D, CD74, HP |
| | C3AR1, IL1R2, CTLA4, CD74, HP |
| | C3AR1, IFNG, CIITA, CD3D, CTLA4 |
| | C3AR1, IFNG, CIITA, CD3D, CD74 |
| | C3AR1, IFNG, CIITA, CD3D, HP |
| | C3AR1, IFNG, CIITA, CTLA4, CD74 |
| | C3AR1, IFNG, CIITA, CTLA4, HP |
| | C3AR1, IFNG, CIITA, CD74, HP |
| | C3AR1, IFNG, CD3D, CTLA4, CD74 |
| | C3AR1, IFNG, CD3D, CTLA4, HP |
| | C3AR1, IFNG, CD3D, CD74, HP |
| | C3AR1, IFNG, CTLA4, CD74, HP |
| | C3AR1, CIITA, CD3D, CTLA4, CD74 |
| | C3AR1, CIITA, CD3D, CTLA4, HP |
| | C3AR1, CIITA, CD3D, CD74, HP |
| | C3AR1, CIITA, CTLA4, CD74, HP |
| | C3AR1, CD3D, CTLA4, CD74, HP |
| | TAP2, CX3CR1, IL1R2, IFNG, CIITA |
| | TAP2, CX3CR1, IL1R2, IFNG, CD3D |
| | TAP2, CX3CR1, IL1R2, IFNG, CTLA4 |
| | TAP2, CX3CR1, IL1R2, IFNG, CD74 |
| | TAP2, CX3CR1, IL1R2, IFNG, HP |
| | TAP2, CX3CR1, IL1R2, CIITA, CD3D |
| | TAP2, CX3CR1, IL1R2, CIITA, CTLA4 |
| | TAP2, CX3CR1, IL1R2, CIITA, CD74 |
| | TAP2, CX3CR1, IL1R2, CIITA, HP |
| | TAP2, CX3CR1, IL1R2, CD3D, CTLA4 |
| | TAP2, CX3CR1, IL1R2, CD3D, CD74 |
| | TAP2, CX3CR1, IL1R2, CD3D, HP |
| | TAP2, CX3CR1, IL1R2, CTLA4, CD74 |
| | TAP2, CX3CR1, IL1R2, CTLA4, HP |
| | TAP2, CX3CR1, IL1R2, CD74, HP |
| | TAP2, CX3CR1, IFNG, CIITA, CD3D |
| | TAP2, CX3CR1, IFNG, CIITA, CTLA4 |
| | TAP2, CX3CR1, IFNG, CIITA, CD74 |
| | TAP2, CX3CR1, IFNG, CIITA, HP |
| | TAP2, CX3CR1, IFNG, CD3D, CTLA4 |
| | TAP2, CX3CR1, IFNG, CD3D, CD74 |
| | TAP2, CX3CR1, IFNG, CD3D, HP |
| | TAP2, CX3CR1, IFNG, CTLA4, CD74 |
| | TAP2, CX3CR1, IFNG, CTLA4, HP |
| | TAP2, CX3CR1, IFNG, CD74, HP |
| | TAP2, CX3CR1, CIITA, CD3D, CTLA4 |
| | TAP2, CX3CR1, CIITA, CD3D, CD74 |
| | TAP2, CX3CR1, CIITA, CD3D, HP |
| | TAP2, CX3CR1, CIITA, CTLA4, CD74 |
| | TAP2, CX3CR1, CIITA, CTLA4, HP |
| | TAP2, CX3CR1, CIITA, CD74, HP |
| | TAP2, CX3CR1, CD3D, CTLA4, CD74 |
| | TAP2, CX3CR1, CD3D, CTLA4, HP |
| | TAP2, CX3CR1, CD3D, CD74, HP |
| | TAP2, CX3CR1, CTLA4, CD74, HP |
| | TAP2, IL1R2, IFNG, CIITA, CD3D |
| | TAP2, IL1R2, IFNG, CIITA, CTLA4 |
| | TAP2, IL1R2, IFNG, CIITA, CD74 |
| | TAP2, IL1R2, IFNG, CIITA, HP |
| | TAP2, IL1R2, IFNG, CD3D, CTLA4 |
| | TAP2, IL1R2, IFNG, CD3D, CD74 |
| | TAP2, IL1R2, IFNG, CD3D, HP |
| | TAP2, IL1R2, IFNG, CTLA4, CD74 |
| | TAP2, IL1R2, IFNG, CTLA4, HP |
| | TAP2, IL1R2, IFNG, CD74, HP |
| | TAP2, IL1R2, CIITA, CD3D, CTLA4 |
| | TAP2, IL1R2, CIITA, CD3D, CD74 |
| | TAP2, IL1R2, CIITA, CD3D, HP |
| | TAP2, IL1R2, CIITA, CTLA4, CD74 |
| | TAP2, IL1R2, CIITA, CTLA4, HP |
| | TAP2, IL1R2, CIITA, CD74, HP |
| | TAP2, IL1R2, CD3D, CTLA4, CD74 |
| | TAP2, IL1R2, CD3D, CTLA4, HP |
| | TAP2, IL1R2, CD3D, CD74, HP |
| | TAP2, IL1R2, CTLA4, CD74, HP |
| | TAP2, IFNG, CIITA, CD3D, CTLA4 |
| | TAP2, IFNG, CIITA, CD3D, CD74 |
| | TAP2, IFNG, CIITA, CD3D, HP |
| | TAP2, IFNG, CIITA, CTLA4, CD74 |
| | TAP2, IFNG, CIITA, CTLA4, HP |
| | TAP2, IFNG, CIITA, CD74, HP |
| | TAP2, IFNG, CD3D, CTLA4, CD74 |
| | TAP2, IFNG, CD3D, CTLA4, HP |
| | TAP2, IFNG, CD3D, CD74, HP |
| | TAP2, IFNG, CTLA4, CD74, HP |
| | TAP2, CIITA, CD3D, CTLA4, CD74 |
| | TAP2, CIITA, CD3D, CTLA4, HP |
| | TAP2, CIITA, CD3D, CD74, HP |
| | TAP2, CIITA, CTLA4, CD74, HP |
| | TAP2, CD3D, CTLA4, CD74, HP |
| | CX3CR1, IL1R2, IFNG, CIITA, CD3D |
| | CX3CR1, IL1R2, IFNG, CIITA, CTLA4 |
| | CX3CR1, IL1R2, IFNG, CIITA, CD74 |
| | CX3CR1, IL1R2, IFNG, CIITA, HP |
| | CX3CR1, IL1R2, IFNG, CD3D, CTLA4 |
| | CX3CR1, IL1R2, IFNG, CD3D, CD74 |
| | CX3CR1, IL1R2, IFNG, CD3D, HP |
| | CX3CR1, IL1R2, IFNG, CTLA4, CD74 |
| | CX3CR1, IL1R2, IFNG, CTLA4, HP |
| | CX3CR1, IL1R2, IFNG, CD74, HP |
| | CX3CR1, IL1R2, CIITA, CD3D, CTLA4 |
| | CX3CR1, IL1R2, CIITA, CD3D, CD74 |
| | CX3CR1, IL1R2, CIITA, CD3D, HP |
| | CX3CR1, IL1R2, CIITA, CTLA4, CD74 |
| | CX3CR1, IL1R2, CIITA, CTLA4, HP |
| | CX3CR1, IL1R2, CIITA, CD74, HP |
| | CX3CR1, IL1R2, CD3D, CTLA4, CD74 |
| | CX3CR1, IL1R2, CD3D, CTLA4, HP |
| | CX3CR1, IL1R2, CD3D, CD74, HP |
| | CX3CR1, IL1R2, CTLA4, CD74, HP |
| | CX3CR1, IFNG, CIITA, CD3D, CTLA4 |
| | CX3CR1, IFNG, CIITA, CD3D, CD74 |
| | CX3CR1, IFNG, CIITA, CD3D, HP |
| | CX3CR1, IFNG, CIITA, CTLA4, CD74 |
| | CX3CR1, IFNG, CIITA, CTLA4, HP |
| | CX3CR1, IFNG, CIITA, CD74, HP |
| | CX3CR1, IFNG, CD3D, CTLA4, CD74 |
| | CX3CR1, IFNG, CD3D, CTLA4, HP |
| | CX3CR1, IFNG, CD3D, CD74, HP |
| | CX3CR1, IFNG, CTLA4, CD74, HP |
| | CX3CR1, CIITA, CD3D, CTLA4, CD74 |
| | CX3CR1, CIITA, CD3D, CTLA4, HP |
| | CX3CR1, CIITA, CD3D, CD74, HP |
| | CX3CR1, CIITA, CTLA4, CD74, HP |
| | CX3CR1, CD3D, CTLA4, CD74, HP |
| | IL1R2, IFNG, CIITA, CD3D, CTLA4 |
| | IL1R2, IFNG, CIITA, CD3D, CD74 |
| | IL1R2, IFNG, CIITA, CD3D, HP |
| | IL1R2, IFNG, CIITA, CTLA4, CD74 |
| | IL1R2, IFNG, CIITA, CTLA4, HP |
| | IL1R2, IFNG, CIITA, CD74, HP |
| | IL1R2, IFNG, CD3D, CTLA4, CD74 |
| | IL1R2, IFNG, CD3D, CTLA4, HP |
| | IL1R2, IFNG, CD3D, CD74, HP |
| | IL1R2, IFNG, CTLA4, CD74, HP |
| | IL1R2, CIITA, CD3D, CTLA4, CD74 |
| | IL1R2, CIITA, CD3D, CTLA4, HP |
| | IL1R2, CIITA, CD3D, CD74, HP |
| | IL1R2, CIITA, CTLA4, CD74, HP |
| | IL1R2, CD3D, CTLA4, CD74, HP |
| | IFNG, CIITA, CD3D, CTLA4, CD74 |
| | IFNG, CIITA, CD3D, CTLA4, HP |
| | IFNG, CIITA, CD3D, CD74, HP |
| | IFNG, CIITA, CTLA4, CD74, HP |
| | IFNG, CD3D, CTLA4, CD74, HP |
| | CIITA, CD3D, CTLA4, CD74, HP |
| Combinations of | S100A9, C3AR1, TAP2, CX3CR1, IL1R2, IFNG |
| six genes | S100A9, C3AR1, TAP2, CX3CR1, IL1R2, CIITA |
| | S100A9, C3AR1, TAP2, CX3CR1, IL1R2, CD3D |
| | S100A9, C3AR1, TAP2, CX3CR1, IL1R2, CTLA4 |
| | S100A9, C3AR1, TAP2, CX3CR1, IL1R2, CD74 |
| | S100A9, C3AR1, TAP2, CX3CR1, IL1R2, HP |
| | S100A9, C3AR1, TAP2, CX3CR1, IFNG, CIITA |
| | S100A9, C3AR1, TAP2, CX3CR1, IFNG, CD3D |
| | S100A9, C3AR1, TAP2, CX3CR1, IFNG, CTLA4 |
| | S100A9, C3AR1, TAP2, CX3CR1, IFNG, CD74 |
| | S100A9, C3AR1, TAP2, CX3CR1, IFNG, HP |
| | S100A9, C3AR1, TAP2, CX3CR1, CIITA, CD3D |
| | S100A9, C3AR1, TAP2, CX3CR1, CIITA, CTLA4 |
| | S100A9, C3AR1, TAP2, CX3CR1, CIITA, CD74 |
| | S100A9, C3AR1, TAP2, CX3CR1, CIITA, HP |
| | S100A9, C3AR1, TAP2, CX3CR1, CD3D, CTLA4 |
| | S100A9, C3AR1, TAP2, CX3CR1, CD3D, CD74 |
| | S100A9, C3AR1, TAP2, CX3CR1, CD3D, HP |
| | S100A9, C3AR1, TAP2, CX3CR1, CTLA4, CD74 |
| | S100A9, C3AR1, TAP2, CX3CR1, CTLA4, HP |
| | S100A9, C3AR1, TAP2, CX3CR1, CD74, HP |
| | S100A9, C3AR1, TAP2, IL1R2, IFNG, CIITA |
| | S100A9, C3AR1, TAP2, IL1R2, IFNG, CD3D |
| | S100A9, C3AR1, TAP2, IL1R2, IFNG, CTLA4 |
| | S100A9, C3AR1, TAP2, IL1R2, IFNG, CD74 |
| | S100A9, C3AR1, TAP2, IL1R2, IFNG, HP |
| | S100A9, C3AR1, TAP2, IL1R2, CIITA, CD3D |
| | S100A9, C3AR1, TAP2, IL1R2, CIITA, CTLA4 |
| | S100A9, C3AR1, TAP2, IL1R2, CIITA, CD74 |
| | S100A9, C3AR1, TAP2, IL1R2, CIITA, HP |
| | S100A9, C3AR1, TAP2, IL1R2, CD3D, CTLA4 |
| | S100A9, C3AR1, TAP2, IL1R2, CD3D, CD74 |
| | S100A9, C3AR1, TAP2, IL1R2, CD3D, HP |
| | S100A9, C3AR1, TAP2, IL1R2, CTLA4, CD74 |
| | S100A9, C3AR1, TAP2, IL1R2, CTLA4, HP |
| | S100A9, C3AR1, TAP2, IL1R2, CD74, HP |
| | S100A9, C3AR1, TAP2, IFNG, CIITA, CD3D |
| | S100A9, C3AR1, TAP2, IFNG, CIITA, CTLA4 |
| | S100A9, C3AR1, TAP2, IFNG, CIITA, CD74 |
| | S100A9, C3AR1, TAP2, IFNG, CIITA, HP |
| | S100A9, C3AR1, TAP2, IFNG, CD3D, CTLA4 |
| | S100A9, C3AR1, TAP2, IFNG, CD3D, CD74 |
| | S100A9, C3AR1, TAP2, IFNG, CD3D, HP |
| | S100A9, C3AR1, TAP2, IFNG, CTLA4, CD74 |
| | S100A9, C3AR1, TAP2, IFNG, CTLA4, HP |
| | S100A9, C3AR1, TAP2, IFNG, CD74, HP |
| | S100A9, C3AR1, TAP2, CIITA, CD3D, CTLA4 |
| | S100A9, C3AR1, TAP2, CIITA, CD3D, CD74 |
| | S100A9, C3AR1, TAP2, CIITA, CD3D, HP |
| | S100A9, C3AR1, TAP2, CIITA, CTLA4, CD74 |
| | S100A9, C3AR1, TAP2, CIITA, CTLA4, HP |
| | S100A9, C3AR1, TAP2, CIITA, CD74, HP |
| | S100A9, C3AR1, TAP2, CD3D, CTLA4, CD74 |
| | S100A9, C3AR1, TAP2, CD3D, CTLA4, HP |
| | S100A9, C3AR1, TAP2, CD3D, CD74, HP |
| | S100A9, C3AR1, TAP2, CTLA4, CD74, HP |
| | S100A9, C3AR1, CX3CR1, IL1R2, IFNG, CIITA |
| | S100A9, C3AR1, CX3CR1, IL1R2, IFNG, CD3D |
| | S100A9, C3AR1, CX3CR1, IL1R2, IFNG, CTLA4 |
| | S100A9, C3AR1, CX3CR1, IL1R2, IFNG, CD74 |
| | S100A9, C3AR1, CX3CR1, IL1R2, IFNG, HP |
| | S100A9, C3AR1, CX3CR1, IL1R2, CIITA, CD3D |
| | S100A9, C3AR1, CX3CR1, IL1R2, CIITA, CTLA4 |
| | S100A9, C3AR1, CX3CR1, IL1R2, CIITA, CD74 |
| | S100A9, C3AR1, CX3CR1, IL1R2, CIITA, HP |
| | S100A9, C3AR1, CX3CR1, IL1R2, CD3D, CTLA4 |
| | S100A9, C3AR1, CX3CR1, IL1R2, CD3D, CD74 |
| | S100A9, C3AR1, CX3CR1, IL1R2, CD3D, HP |
| | S100A9, C3AR1, CX3CR1, IL1R2, CTLA4, CD74 |
| | S100A9, C3AR1, CX3CR1, IL1R2, CTLA4, HP |
| | S100A9, C3AR1, CX3CR1, IL1R2, CD74, HP |
| | S100A9, C3AR1, CX3CR1, IFNG, CIITA, CD3D |
| | S100A9, C3AR1, CX3CR1, IFNG, CIITA, CTLA4 |
| | S100A9, C3AR1, CX3CR1, IFNG, CIITA, CD74 |
| | S100A9, C3AR1, CX3CR1, IFNG, CIITA, HP |
| | S100A9, C3AR1, CX3CR1, IFNG, CD3D, CTLA4 |
| | S100A9, C3AR1, CX3CR1, IFNG, CD3D, CD74 |
| | S100A9, C3AR1, CX3CR1, IFNG, CD3D, HP |
| | S100A9, C3AR1, CX3CR1, IFNG, CTLA4, CD74 |
| | S100A9, C3AR1, CX3CR1, IFNG, CTLA4, HP |
| | S100A9, C3AR1, CX3CR1, IFNG, CD74, HP |
| | S100A9, C3AR1, CX3CR1, CIITA, CD3D, CTLA4 |
| | S100A9, C3AR1, CX3CR1, CIITA, CD3D, CD74 |
| | S100A9, C3AR1, CX3CR1, CIITA, CD3D, HP |
| | S100A9, C3AR1, CX3CR1, CIITA, CTLA4, CD74 |
| | S100A9, C3AR1, CX3CR1, CIITA, CTLA4, HP |
| | S100A9, C3AR1, CX3CR1, CIITA, CD74, HP |
| | S100A9, C3AR1, CX3CR1, CD3D, CTLA4, CD74 |
| | S100A9, C3AR1, CX3CR1, CD3D, CTLA4, HP |
| | S100A9, C3AR1, CX3CR1, CD3D, CD74, HP |
| | S100A9, C3AR1, CX3CR1, CTLA4, CD74, HP |
| | S100A9, C3AR1, IL1R2, IFNG, CIITA, CD3D |
| | S100A9, C3AR1, IL1R2, IFNG, CIITA, CTLA4 |
| | S100A9, C3AR1, IL1R2, IFNG, CIITA, CD74 |
| | S100A9, C3AR1, IL1R2, IFNG, CIITA, HP |
| | S100A9, C3AR1, IL1R2, IFNG, CD3D, CTLA4 |
| | S100A9, C3AR1, IL1R2, IFNG, CD3D, CD74 |
| | S100A9, C3AR1, IL1R2, IFNG, CD3D, HP |
| | S100A9, C3AR1, IL1R2, IFNG, CTLA4, CD74 |
| | S100A9, C3AR1, IL1R2, IFNG, CTLA4, HP |
| | S100A9, C3AR1, IL1R2, IFNG, CD74, HP |
| | S100A9, C3AR1, IL1R2, CIITA, CD3D, CTLA4 |
| | S100A9, C3AR1, IL1R2, CIITA, CD3D, CD74 |
| | S100A9, C3AR1, IL1R2, CIITA, CD3D, HP |
| | S100A9, C3AR1, IL1R2, CIITA, CTLA4, CD74 |
| | S100A9, C3AR1, IL1R2, CIITA, CTLA4, HP |
| | S100A9, C3AR1, IL1R2, CIITA, CD74, HP |
| | S100A9, C3AR1, IL1R2, CD3D, CTLA4, CD74 |
| | S100A9, C3AR1, IL1R2, CD3D, CTLA4, HP |
| | S100A9, C3AR1, IL1R2, CD3D, CD74, HP |
| | S100A9, C3AR1, IL1R2, CTLA4, CD74, HP |
| | S100A9, C3AR1, IFNG, CIITA, CD3D, CTLA4 |
| | S100A9, C3AR1, IFNG, CIITA, CD3D, CD74 |
| | S100A9, C3AR1, IFNG, CIITA, CD3D, HP |
| | S100A9, C3AR1, IFNG, CIITA, CTLA4, CD74 |
| | S100A9, C3AR1, IFNG, CIITA, CTLA4, HP |
| | S100A9, C3AR1, IFNG, CIITA, CD74, HP |
| | S100A9, C3AR1, IFNG, CD3D, CTLA4, CD74 |
| | S100A9, C3AR1, IFNG, CD3D, CTLA4, HP |
| | S100A9, C3AR1, IFNG, CD3D, CD74, HP |
| | S100A9, C3AR1, IFNG, CTLA4, CD74, HP |
| | S100A9, C3AR1, CIITA, CD3D, CTLA4, CD74 |
| | S100A9, C3AR1, CIITA, CD3D, CTLA4, HP |
| | S100A9, C3AR1, CIITA, CD3D, CD74, HP |
| | S100A9, C3AR1, CIITA, CTLA4, CD74, HP |
| | S100A9, C3AR1, CD3D, CTLA4, CD74, HP |
| | S100A9, TAP2, CX3CR1, IL1R2, IFNG, CIITA |
| | S100A9, TAP2, CX3CR1, IL1R2, IFNG, CD3D |
| | S100A9, TAP2, CX3CR1, IL1R2, IFNG, CTLA4 |
| | S100A9, TAP2, CX3CR1, IL1R2, IFNG, CD74 |
| | S100A9, TAP2, CX3CR1, IL1R2, IFNG, HP |
| | S100A9, TAP2, CX3CR1, IL1R2, CIITA, CD3D |
| | S100A9, TAP2, CX3CR1, IL1R2, CIITA, CTLA4 |
| | S100A9, TAP2, CX3CR1, IL1R2, CIITA, CD74 |
| | S100A9, TAP2, CX3CR1, IL1R2, CIITA, HP |
| | S100A9, TAP2, CX3CR1, IL1R2, CD3D, CTLA4 |
| | S100A9, TAP2, CX3CR1, IL1R2, CD3D, CD74 |
| | S100A9, TAP2, CX3CR1, IL1R2, CD3D, HP |
| | S100A9, TAP2, CX3CR1, IL1R2, CTLA4, CD74 |
| | S100A9, TAP2, CX3CR1, IL1R2, CTLA4, HP |
| | S100A9, TAP2, CX3CR1, IL1R2, CD74, HP |
| | S100A9, TAP2, CX3CR1, IFNG, CIITA, CD3D |
| | S100A9, TAP2, CX3CR1, IFNG, CIITA, CTLA4 |
| | S100A9, TAP2, CX3CR1, IFNG, CIITA, CD74 |
| | S100A9, TAP2, CX3CR1, IFNG, CIITA, HP |
| | S100A9, TAP2, CX3CR1, IFNG, CD3D, CTLA4 |
| | S100A9, TAP2, CX3CR1, IFNG, CD3D, CD74 |
| | S100A9, TAP2, CX3CR1, IFNG, CD3D, HP |
| | S100A9, TAP2, CX3CR1, IFNG, CTLA4, CD74 |
| | S100A9, TAP2, CX3CR1, IFNG, CTLA4, HP |
| | S100A9, TAP2, CX3CR1, IFNG, CD74, HP |
| | S100A9, TAP2, CX3CR1, CIITA, CD3D, CTLA4 |
| | S100A9, TAP2, CX3CR1, CIITA, CD3D, CD74 |
| | S100A9, TAP2, CX3CR1, CIITA, CD3D, HP |
| | S100A9, TAP2, CX3CR1, CIITA, CTLA4, CD74 |
| | S100A9, TAP2, CX3CR1, CIITA, CTLA4, HP |
| | S100A9, TAP2, CX3CR1, CIITA, CD74, HP |
| | S100A9, TAP2, CX3CR1, CD3D, CTLA4, CD74 |
| | S100A9, TAP2, CX3CR1, CD3D, CTLA4, HP |
| | S100A9, TAP2, CX3CR1, CD3D, CD74, HP |
| | S100A9, TAP2, CX3CR1, CTLA4, CD74, HP |
| | S100A9, TAP2, IL1R2, IFNG, CIITA, CD3D |
| | S100A9, TAP2, IL1R2, IFNG, CIITA, CTLA4 |
| | S100A9, TAP2, IL1R2, IFNG, CIITA, CD74 |
| | S100A9, TAP2, IL1R2, IFNG, CIITA, HP |
| | S100A9, TAP2, IL1R2, IFNG, CD3D, CTLA4 |
| | S100A9, TAP2, IL1R2, IFNG, CD3D, CD74 |
| | S100A9, TAP2, IL1R2, IFNG, CD3D, HP |
| | S100A9, TAP2, IL1R2, IFNG, CTLA4, CD74 |
| | S100A9, TAP2, IL1R2, IFNG, CTLA4, HP |
| | S100A9, TAP2, IL1R2, IFNG, CD74, HP |
| | S100A9, TAP2, IL1R2, CIITA, CD3D, CTLA4 |
| | S100A9, TAP2, IL1R2, CIITA, CD3D, CD74 |
| | S100A9, TAP2, IL1R2, CIITA, CD3D, HP |
| | S100A9, TAP2, IL1R2, CIITA, CTLA4, CD74 |
| | S100A9, TAP2, IL1R2, CIITA, CTLA4, HP |
| | S100A9, TAP2, IL1R2, CIITA, CD74, HP |
| | S100A9, TAP2, IL1R2, CD3D, CTLA4, CD74 |
| | S100A9, TAP2, IL1R2, CD3D, CTLA4, HP |
| | S100A9, TAP2, IL1R2, CD3D, CD74, HP |
| | S100A9, TAP2, IL1R2, CTLA4, CD74, HP |
| | S100A9, TAP2, IFNG, CIITA, CD3D, CTLA4 |
| | S100A9, TAP2, IFNG, CIITA, CD3D, CD74 |
| | S100A9, TAP2, IFNG, CIITA, CD3D, HP |
| | S100A9, TAP2, IFNG, CIITA, CTLA4, CD74 |
| | S100A9, TAP2, IFNG, CIITA, CTLA4, HP |
| | S100A9, TAP2, IFNG, CIITA, CD74, HP |
| | S100A9, TAP2, IFNG, CD3D, CTLA4, CD74 |
| | S100A9, TAP2, IFNG, CD3D, CTLA4, HP |
| | S100A9, TAP2, IFNG, CD3D, CD74, HP |
| | S100A9, TAP2, IFNG, CTLA4, CD74, HP |
| | S100A9, TAP2, CIITA, CD3D, CTLA4, CD74 |
| | S100A9, TAP2, CIITA, CD3D, CTLA4, HP |
| | S100A9, TAP2, CIITA, CD3D, CD74, HP |
| | S100A9, TAP2, CIITA, CTLA4, CD74, HP |
| | S100A9, TAP2, CD3D, CTLA4, CD74, HP |
| | S100A9, CX3CR1, IL1R2, IFNG, CIITA, CD3D |
| | S100A9, CX3CR1, IL1R2, IFNG, CIITA, CTLA4 |
| | S100A9, CX3CR1, IL1R2, IFNG, CIITA, CD74 |
| | S100A9, CX3CR1, IL1R2, IFNG, CIITA, HP |
| | S100A9, CX3CR1, IL1R2, IFNG, CD3D, CTLA4 |
| | S100A9, CX3CR1, IL1R2, IFNG, CD3D, CD74 |
| | S100A9, CX3CR1, IL1R2, IFNG, CD3D, HP |
| | S100A9, CX3CR1, IL1R2, IFNG, CTLA4, CD74 |
| | S100A9, CX3CR1, IL1R2, IFNG, CTLA4, HP |
| | S100A9, CX3CR1, IL1R2, IFNG, CD74, HP |
| | S100A9, CX3CR1, IL1R2, CIITA, CD3D, CTLA4 |
| | S100A9, CX3CR1, IL1R2, CIITA, CD3D, CD74 |
| | S100A9, CX3CR1, IL1R2, CIITA, CD3D, HP |
| | S100A9, CX3CR1, IL1R2, CIITA, CTLA4, CD74 |
| | S100A9, CX3CR1, IL1R2, CIITA, CTLA4, HP |
| | S100A9, CX3CR1, IL1R2, CIITA, CD74, HP |
| | S100A9, CX3CR1, IL1R2, CD3D, CTLA4, CD74 |
| | S100A9, CX3CR1, IL1R2, CD3D, CTLA4, HP |
| | S100A9, CX3CR1, IL1R2, CD3D, CD74, HP |
| | S100A9, CX3CR1, IL1R2, CTLA4, CD74, HP |
| | S100A9, CX3CR1, IFNG, CIITA, CD3D, CTLA4 |
| | S100A9, CX3CR1, IFNG, CIITA, CD3D, CD74 |
| | S100A9, CX3CR1, IFNG, CIITA, CD3D, HP |
| | S100A9, CX3CR1, IFNG, CIITA, CTLA4, CD74 |
| | S100A9, CX3CR1, IFNG, CIITA, CTLA4, HP |
| | S100A9, CX3CR1, IFNG, CIITA, CD74, HP |
| | S100A9, CX3CR1, IFNG, CD3D, CTLA4, CD74 |
| | S100A9, CX3CR1, IFNG, CD3D, CTLA4, HP |
| | S100A9, CX3CR1, IFNG, CD3D, CD74, HP |
| | S100A9, CX3CR1, IFNG, CTLA4, CD74, HP |
| | S100A9, CX3CR1, CIITA, CD3D, CTLA4, CD74 |
| | S100A9, CX3CR1, CIITA, CD3D, CTLA4, HP |
| | S100A9, CX3CR1, CIITA, CD3D, CD74, HP |
| | S100A9, CX3CR1, CIITA, CTLA4, CD74, HP |
| | S100A9, CX3CR1, CD3D, CTLA4, CD74, HP |
| | S100A9, IL1R2, IFNG, CIITA, CD3D, CTLA4 |
| | S100A9, IL1R2, IFNG, CIITA, CD3D, CD74 |
| | S100A9, IL1R2, IFNG, CIITA, CD3D, HP |
| | S100A9, IL1R2, IFNG, CIITA, CTLA4, CD74 |
| | S100A9, IL1R2, IFNG, CIITA, CTLA4, HP |
| | S100A9, IL1R2, IFNG, CIITA, CD74, HP |
| | S100A9, IL1R2, IFNG, CD3D, CTLA4, CD74 |
| | S100A9, IL1R2, IFNG, CD3D, CTLA4, HP |
| | S100A9, IL1R2, IFNG, CD3D, CD74, HP |
| | S100A9, IL1R2, IFNG, CTLA4, CD74, HP |
| | S100A9, IL1R2, CIITA, CD3D, CTLA4, CD74 |
| | S100A9, IL1R2, CIITA, CD3D, CTLA4, HP |
| | S100A9, IL1R2, CIITA, CD3D, CD74, HP |
| | S100A9, IL1R2, CIITA, CTLA4, CD74, HP |
| | S100A9, IL1R2, CD3D, CTLA4, CD74, HP |
| | S100A9, IFNG, CIITA, CD3D, CTLA4, CD74 |
| | S100A9, IFNG, CIITA, CD3D, CTLA4, HP |
| | S100A9, IFNG, CIITA, CD3D, CD74, HP |
| | S100A9, IFNG, CIITA, CTLA4, CD74, HP |
| | S100A9, IFNG, CD3D, CTLA4, CD74, HP |
| | S100A9, CIITA, CD3D, CTLA4, CD74, HP |
| | C3AR1, TAP2, CX3CR1, IL1R2, IFNG, CIITA |
| | C3AR1, TAP2, CX3CR1, IL1R2, IFNG, CD3D |
| | C3AR1, TAP2, CX3CR1, IL1R2, IFNG, CTLA4 |
| | C3AR1, TAP2, CX3CR1, IL1R2, IFNG, CD74 |
| | C3AR1, TAP2, CX3CR1, IL1R2, IFNG, HP |
| | C3AR1, TAP2, CX3CR1, IL1R2, CIITA, CD3D |
| | C3AR1, TAP2, CX3CR1, IL1R2, CIITA, CTLA4 |
| | C3AR1, TAP2, CX3CR1, IL1R2, CIITA, CD74 |
| | C3AR1, TAP2, CX3CR1, IL1R2, CIITA, HP |
| | C3AR1, TAP2, CX3CR1, IL1R2, CD3D, CTLA4 |
| | C3AR1, TAP2, CX3CR1, IL1R2, CD3D, CD74 |
| | C3AR1, TAP2, CX3CR1, IL1R2, CD3D, HP |
| | C3AR1, TAP2, CX3CR1, IL1R2, CTLA4, CD74 |
| | C3AR1, TAP2, CX3CR1, IL1R2, CTLA4, HP |
| | C3AR1, TAP2, CX3CR1, IL1R2, CD74, HP |
| | C3AR1, TAP2, CX3CR1, IFNG, CIITA, CD3D |
| | C3AR1, TAP2, CX3CR1, IFNG, CIITA, CTLA4 |
| | C3AR1, TAP2, CX3CR1, IFNG, CIITA, CD74 |
| | C3AR1, TAP2, CX3CR1, IFNG, CIITA, HP |
| | C3AR1, TAP2, CX3CR1, IFNG, CD3D, CTLA4 |
| | C3AR1, TAP2, CX3CR1, IFNG, CD3D, CD74 |
| | C3AR1, TAP2, CX3CR1, IFNG, CD3D, HP |
| | C3AR1, TAP2, CX3CR1, IFNG, CTLA4, CD74 |
| | C3AR1, TAP2, CX3CR1, IFNG, CTLA4, HP |
| | C3AR1, TAP2, CX3CR1, IFNG, CD74, HP |
| | C3AR1, TAP2, CX3CR1, CIITA, CD3D, CTLA4 |
| | C3AR1, TAP2, CX3CR1, CIITA, CD3D, CD74 |
| | C3AR1, TAP2, CX3CR1, CIITA, CD3D, HP |
| | C3AR1, TAP2, CX3CR1, CIITA, CTLA4, CD74 |
| | C3AR1, TAP2, CX3CR1, CIITA, CTLA4, HP |
| | C3AR1, TAP2, CX3CR1, CIITA, CD74, HP |
| | C3AR1, TAP2, CX3CR1, CD3D, CTLA4, CD74 |
| | C3AR1, TAP2, CX3CR1, CD3D, CTLA4, HP |
| | C3AR1, TAP2, CX3CR1, CD3D, CD74, HP |
| | C3AR1, TAP2, CX3CR1, CTLA4, CD74, HP |
| | C3AR1, TAP2, IL1R2, IFNG, CIITA, CD3D |
| | C3AR1, TAP2, IL1R2, IFNG, CIITA, CTLA4 |
| | C3AR1, TAP2, IL1R2, IFNG, CIITA, CD74 |
| | C3AR1, TAP2, IL1R2, IFNG, CIITA, HP |
| | C3AR1, TAP2, IL1R2, IFNG, CD3D, CTLA4 |
| | C3AR1, TAP2, IL1R2, IFNG, CD3D, CD74 |
| | C3AR1, TAP2, IL1R2, IFNG, CD3D, HP |
| | C3AR1, TAP2, IL1R2, IFNG, CTLA4, CD74 |
| | C3AR1, TAP2, IL1R2, IFNG, CTLA4, HP |
| | C3AR1, TAP2, IL1R2, IFNG, CD74, HP |
| | C3AR1, TAP2, IL1R2, CIITA, CD3D, CTLA4 |
| | C3AR1, TAP2, IL1R2, CIITA, CD3D, CD74 |
| | C3AR1, TAP2, IL1R2, CIITA, CD3D, HP |
| | C3AR1, TAP2, IL1R2, CIITA, CTLA4, CD74 |
| | C3AR1, TAP2, IL1R2, CIITA, CTLA4, HP |
| | C3AR1, TAP2, IL1R2, CIITA, CD74, HP |
| | C3AR1, TAP2, IL1R2, CD3D, CTLA4, CD74 |
| | C3AR1, TAP2, IL1R2, CD3D, CTLA4, HP |
| | C3AR1, TAP2, IL1R2, CD3D, CD74, HP |
| | C3AR1, TAP2, IL1R2, CTLA4, CD74, HP |
| | C3AR1, TAP2, IFNG, CIITA, CD3D, CTLA4 |
| | C3AR1, TAP2, IFNG, CIITA, CD3D, CD74 |
| | C3AR1, TAP2, IFNG, CIITA, CD3D, HP |
| | C3AR1, TAP2, IFNG, CIITA, CTLA4, CD74 |
| | C3AR1, TAP2, IFNG, CIITA, CTLA4, HP |
| | C3AR1, TAP2, IFNG, CIITA, CD74, HP |
| | C3AR1, TAP2, IFNG, CD3D, CTLA4, CD74 |
| | C3AR1, TAP2, IFNG, CD3D, CTLA4, HP |
| | C3AR1, TAP2, IFNG, CD3D, CD74, HP |
| | C3AR1, TAP2, IFNG, CTLA4, CD74, HP |
| | C3AR1, TAP2, CIITA, CD3D, CTLA4, CD74 |
| | C3AR1, TAP2, CIITA, CD3D, CTLA4, HP |
| | C3AR1, TAP2, CIITA, CD3D, CD74, HP |
| | C3AR1, TAP2, CIITA, CTLA4, CD74, HP |
| | C3AR1, TAP2, CD3D, CTLA4, CD74, HP |
| | C3AR1, CX3CR1, IL1R2, IFNG, CIITA, CD3D |
| | C3AR1, CX3CR1, IL1R2, IFNG, CIITA, CTLA4 |
| | C3AR1, CX3CR1, IL1R2, IFNG, CIITA, CD74 |
| | C3AR1, CX3CR1, IL1R2, IFNG, CIITA, HP |
| | C3AR1, CX3CR1, IL1R2, IFNG, CD3D, CTLA4 |
| | C3AR1, CX3CR1, IL1R2, IFNG, CD3D, CD74 |
| | C3AR1, CX3CR1, IL1R2, IFNG, CD3D, HP |
| | C3AR1, CX3CR1, IL1R2, IFNG, CTLA4, CD74 |
| | C3AR1, CX3CR1, IL1R2, IFNG, CTLA4, HP |
| | C3AR1, CX3CR1, IL1R2, IFNG, CD74, HP |
| | C3AR1, CX3CR1, IL1R2, CIITA, CD3D, CTLA4 |
| | C3AR1, CX3CR1, IL1R2, CIITA, CD3D, CD74 |
| | C3AR1, CX3CR1, IL1R2, CIITA, CD3D, HP |
| | C3AR1, CX3CR1, IL1R2, CIITA, CTLA4, CD74 |
| | C3AR1, CX3CR1, IL1R2, CIITA, CTLA4, HP |
| | C3AR1, CX3CR1, IL1R2, CIITA, CD74, HP |
| | C3AR1, CX3CR1, IL1R2, CD3D, CTLA4, CD74 |
| | C3AR1, CX3CR1, IL1R2, CD3D, CTLA4, HP |
| | C3AR1, CX3CR1, IL1R2, CD3D, CD74, HP |
| | C3AR1, CX3CR1, IL1R2, CTLA4, CD74, HP |
| | C3AR1, CX3CR1, IFNG, CIITA, CD3D, CTLA4 |
| | C3AR1, CX3CR1, IFNG, CIITA, CD3D, CD74 |
| | C3AR1, CX3CR1, IFNG, CIITA, CD3D, HP |
| | C3AR1, CX3CR1, IFNG, CIITA, CTLA4, CD74 |
| | C3AR1, CX3CR1, IFNG, CIITA, CTLA4, HP |
| | C3AR1, CX3CR1, IFNG, CIITA, CD74, HP |
| | C3AR1, CX3CR1, IFNG, CD3D, CTLA4, CD74 |
| | C3AR1, CX3CR1, IFNG, CD3D, CTLA4, HP |
| | C3AR1, CX3CR1, IFNG, CD3D, CD74, HP |
| | C3AR1, CX3CR1, IFNG, CTLA4, CD74, HP |
| | C3AR1, CX3CR1, CIITA, CD3D, CTLA4, CD74 |
| | C3AR1, CX3CR1, CIITA, CD3D, CTLA4, HP |
| | C3AR1, CX3CR1, CIITA, CD3D, CD74, HP |
| | C3AR1, CX3CR1, CIITA, CTLA4, CD74, HP |
| | C3AR1, CX3CR1, CD3D, CTLA4, CD74, HP |
| | C3AR1, IL1R2, IFNG, CIITA, CD3D, CTLA4 |
| | C3AR1, IL1R2, IFNG, CIITA, CD3D, CD74 |
| | C3AR1, IL1R2, IFNG, CIITA, CD3D, HP |
| | C3AR1, IL1R2, IFNG, CIITA, CTLA4, CD74 |
| | C3AR1, IL1R2, IFNG, CIITA, CTLA4, HP |
| | C3AR1, IL1R2, IFNG, CIITA, CD74, HP |
| | C3AR1, IL1R2, IFNG, CD3D, CTLA4, CD74 |
| | C3AR1, IL1R2, IFNG, CD3D, CTLA4, HP |
| | C3AR1, IL1R2, IFNG, CD3D, CD74, HP |
| | C3AR1, IL1R2, IFNG, CTLA4, CD74, HP |
| | C3AR1, IL1R2, CIITA, CD3D, CTLA4, CD74 |
| | C3AR1, IL1R2, CIITA, CD3D, CTLA4, HP |
| | C3AR1, IL1R2, CIITA, CD3D, CD74, HP |
| | C3AR1, IL1R2, CIITA, CTLA4, CD74, HP |
| | C3AR1, IL1R2, CD3D, CTLA4, CD74, HP |
| | C3AR1, IFNG, CIITA, CD3D, CTLA4, CD74 |
| | C3AR1, IFNG, CIITA, CD3D, CTLA4, HP |
| | C3AR1, IFNG, CIITA, CD3D, CD74, HP |
| | C3AR1, IFNG, CIITA, CTLA4, CD74, HP |
| | C3AR1, IFNG, CD3D, CTLA4, CD74, HP |
| | C3AR1, CIITA, CD3D, CTLA4, CD74, HP |
| | TAP2, CX3CR1, IL1R2, IFNG, CIITA, CD3D |
| | TAP2, CX3CR1, IL1R2, IFNG, CIITA, CTLA4 |
| | TAP2, CX3CR1, IL1R2, IFNG, CIITA, CD74 |
| | TAP2, CX3CR1, IL1R2, IFNG, CIITA, HP |
| | TAP2, CX3CR1, IL1R2, IFNG, CD3D, CTLA4 |
| | TAP2, CX3CR1, IL1R2, IFNG, CD3D, CD74 |
| | TAP2, CX3CR1, IL1R2, IFNG, CD3D, HP |
| | TAP2, CX3CR1, IL1R2, IFNG, CTLA4, CD74 |
| | TAP2, CX3CR1, IL1R2, IFNG, CTLA4, HP |
| | TAP2, CX3CR1, IL1R2, IFNG, CD74, HP |
| | TAP2, CX3CR1, IL1R2, CIITA, CD3D, CTLA4 |
| | TAP2, CX3CR1, IL1R2, CIITA, CD3D, CD74 |
| | TAP2, CX3CR1, IL1R2, CIITA, CD3D, HP |
| | TAP2, CX3CR1, IL1R2, CIITA, CTLA4, CD74 |
| | TAP2, CX3CR1, IL1R2, CIITA, CTLA4, HP |
| | TAP2, CX3CR1, IL1R2, CIITA, CD74, HP |
| | TAP2, CX3CR1, IL1R2, CD3D, CTLA4, CD74 |
| | TAP2, CX3CR1, IL1R2, CD3D, CTLA4, HP |
| | TAP2, CX3CR1, IL1R2, CD3D, CD74, HP |
| | TAP2, CX3CR1, IL1R2, CTLA4, CD74, HP |
| | TAP2, CX3CR1, IFNG, CIITA, CD3D, CTLA4 |
| | TAP2, CX3CR1, IFNG, CIITA, CD3D, CD74 |
| | TAP2, CX3CR1, IFNG, CIITA, CD3D, HP |
| | TAP2, CX3CR1, IFNG, CIITA, CTLA4, CD74 |
| | TAP2, CX3CR1, IFNG, CIITA, CTLA4, HP |
| | TAP2, CX3CR1, IFNG, CIITA, CD74, HP |
| | TAP2, CX3CR1, IFNG, CD3D, CTLA4, CD74 |
| | TAP2, CX3CR1, IFNG, CD3D, CTLA4, HP |
| | TAP2, CX3CR1, IFNG, CD3D, CD74, HP |
| | TAP2, CX3CR1, IFNG, CTLA4, CD74, HP |
| | TAP2, CX3CR1, CIITA, CD3D, CTLA4, CD74 |
| | TAP2, CX3CR1, CIITA, CD3D, CTLA4, HP |
| | TAP2, CX3CR1, CIITA, CD3D, CD74, HP |
| | TAP2, CX3CR1, CIITA, CTLA4, CD74, HP |
| | TAP2, CX3CR1, CD3D, CTLA4, CD74, HP |
| | TAP2, IL1R2, IFNG, CIITA, CD3D, CTLA4 |
| | TAP2, IL1R2, IFNG, CIITA, CD3D, CD74 |
| | TAP2, IL1R2, IFNG, CIITA, CD3D, HP |
| | TAP2, IL1R2, IFNG, CIITA, CTLA4, CD74 |
| | TAP2, IL1R2, IFNG, CIITA, CTLA4, HP |
| | TAP2, IL1R2, IFNG, CIITA, CD74, HP |
| | TAP2, IL1R2, IFNG, CD3D, CTLA4, CD74 |
| | TAP2, IL1R2, IFNG, CD3D, CTLA4, HP |
| | TAP2, IL1R2, IFNG, CD3D, CD74, HP |
| | TAP2, IL1R2, IFNG, CTLA4, CD74, HP |
| | TAP2, IL1R2, CIITA, CD3D, CTLA4, CD74 |
| | TAP2, IL1R2, CIITA, CD3D, CTLA4, HP |
| | TAP2, IL1R2, CIITA, CD3D, CD74, HP |
| | TAP2, IL1R2, CIITA, CTLA4, CD74, HP |
| | TAP2, IL1R2, CD3D, CTLA4, CD74, HP |
| | TAP2, IFNG, CIITA, CD3D, CTLA4, CD74 |
| | TAP2, IFNG, CIITA, CD3D, CTLA4, HP |
| | TAP2, IFNG, CIITA, CD3D, CD74, HP |
| | TAP2, IFNG, CIITA, CTLA4, CD74, HP |
| | TAP2, IFNG, CD3D, CTLA4, CD74, HP |
| | TAP2, CIITA, CD3D, CTLA4, CD74, HP |
| | CX3CR1, IL1R2, IFNG, CIITA, CD3D, CTLA4 |
| | CX3CR1, IL1R2, IFNG, CIITA, CD3D, CD74 |
| | CX3CR1, IL1R2, IFNG, CIITA, CD3D, HP |
| | CX3CR1, IL1R2, IFNG, CIITA, CTLA4, CD74 |
| | CX3CR1, IL1R2, IFNG, CIITA, CTLA4, HP |
| | CX3CR1, IL1R2, IFNG, CIITA, CD74, HP |
| | CX3CR1, IL1R2, IFNG, CD3D, CTLA4, CD74 |
| | CX3CR1, IL1R2, IFNG, CD3D, CTLA4, HP |
| | CX3CR1, IL1R2, IFNG, CD3D, CD74, HP |
| | CX3CR1, IL1R2, IFNG, CTLA4, CD74, HP |
| | CX3CR1, IL1R2, CIITA, CD3D, CTLA4, CD74 |
| | CX3CR1, IL1R2, CIITA, CD3D, CTLA4, HP |
| | CX3CR1, IL1R2, CIITA, CD3D, CD74, HP |
| | CX3CR1, IL1R2, CIITA, CTLA4, CD74, HP |
| | CX3CR1, IL1R2, CD3D, CTLA4, CD74, HP |
| | CX3CR1, IFNG, CIITA, CD3D, CTLA4, CD74 |
| | CX3CR1, IFNG, CIITA, CD3D, CTLA4, HP |
| | CX3CR1, IFNG, CIITA, CD3D, CD74, HP |
| | CX3CR1, IFNG, CIITA, CTLA4, CD74, HP |
| | CX3CR1, IFNG, CD3D, CTLA4, CD74, HP |
| | CX3CR1, CIITA, CD3D, CTLA4, CD74, HP |
| | IL1R2, IFNG, CIITA, CD3D, CTLA4, CD74 |
| | IL1R2, IFNG, CIITA, CD3D, CTLA4, HP |
| | IL1R2, IFNG, CIITA, CD3D, CD74, HP |
| | IL1R2, IFNG, CIITA, CTLA4, CD74, HP |
| | IL1R2, IFNG, CD3D, CTLA4, CD74, HP |
| | IL1R2, CIITA, CD3D, CTLA4, CD74, HP |
| | IFNG, CIITA, CD3D, CTLA4, CD74, HP |
| Combinations of | S100A9, C3AR1, TAP2, CX3CR1, IL1R2, IFNG, CIITA |
| seven genes | S100A9, C3AR1, TAP2, CX3CR1, IL1R2, IFNG, CD3D |
| | S100A9, C3AR1, TAP2, CX3CR1, IL1R2, IFNG, CTLA4 |
| | S100A9, C3AR1, TAP2, CX3CR1, IL1R2, IFNG, CD74 |
| | S100A9, C3AR1, TAP2, CX3CR1, IL1R2, IFNG, HP |
| | S100A9, C3AR1, TAP2, CX3CR1, IL1R2, CIITA, CD3D |
| | S100A9, C3AR1, TAP2, CX3CR1, IL1R2, CIITA, CTLA4 |
| | S100A9, C3AR1, TAP2, CX3CR1, IL1R2, CIITA, CD74 |
| | S100A9, C3AR1, TAP2, CX3CR1, IL1R2, CIITA, HP |
| | S100A9, C3AR1, TAP2, CX3CR1, IL1R2, CD3D, CTLA4 |
| | S100A9, C3AR1, TAP2, CX3CR1, IL1R2, CD3D, CD74 |
| | S100A9, C3AR1, TAP2, CX3CR1, IL1R2, CD3D, HP |
| | S100A9, C3AR1, TAP2, CX3CR1, IL1R2, CTLA4, CD74 |
| | S100A9, C3AR1, TAP2, CX3CR1, IL1R2, CTLA4, HP |
| | S100A9, C3AR1, TAP2, CX3CR1, IL1R2, CD74, HP |
| | S100A9, C3AR1, TAP2, CX3CR1, IFNG, CIITA, CD3D |
| | S100A9, C3AR1, TAP2, CX3CR1, IFNG, CIITA, CTLA4 |
| | S100A9, C3AR1, TAP2, CX3CR1, IFNG, CIITA, CD74 |
| | S100A9, C3AR1, TAP2, CX3CR1, IFNG, CIITA, HP |
| | S100A9, C3AR1, TAP2, CX3CR1, IFNG, CD3D, CTLA4 |
| | S100A9, C3AR1, TAP2, CX3CR1, IFNG, CD3D, CD74 |
| | S100A9, C3AR1, TAP2, CX3CR1, IFNG, CD3D, HP |
| | S100A9, C3AR1, TAP2, CX3CR1, IFNG, CTLA4, CD74 |
| | S100A9, C3AR1, TAP2, CX3CR1, IFNG, CTLA4, HP |
| | S100A9, C3AR1, TAP2, CX3CR1, IFNG, CD74, HP |
| | S100A9, C3AR1, TAP2, CX3CR1, CIITA, CD3D, CTLA4 |
| | S100A9, C3AR1, TAP2, CX3CR1, CIITA, CD3D, CD74 |
| | S100A9, C3AR1, TAP2, CX3CR1, CIITA, CD3D, HP |
| | S100A9, C3AR1, TAP2, CX3CR1, CIITA, CTLA4, CD74 |
| | S100A9, C3AR1, TAP2, CX3CR1, CIITA, CTLA4, HP |
| | S100A9, C3AR1, TAP2, CX3CR1, CIITA, CD74, HP |
| | S100A9, C3AR1, TAP2, CX3CR1, CD3D, CTLA4, CD74 |
| | S100A9, C3AR1, TAP2, CX3CR1, CD3D, CTLA4, HP |
| | S100A9, C3AR1, TAP2, CX3CR1, CD3D, CD74, HP |
| | S100A9, C3AR1, TAP2, CX3CR1, CTLA4, CD74, HP |
| | S100A9, C3AR1, TAP2, IL1R2, IFNG, CIITA, CD3D |
| | S100A9, C3AR1, TAP2, IL1R2, IFNG, CIITA, CTLA4 |
| | S100A9, C3AR1, TAP2, IL1R2, IFNG, CIITA, CD74 |
| | S100A9, C3AR1, TAP2, IL1R2, IFNG, CIITA, HP |
| | S100A9, C3AR1, TAP2, IL1R2, IFNG, CD3D, CTLA4 |
| | S100A9, C3AR1, TAP2, IL1R2, IFNG, CD3D, CD74 |
| | S100A9, C3AR1, TAP2, IL1R2, IFNG, CD3D, HP |
| | S100A9, C3AR1, TAP2, IL1R2, IFNG, CTLA4, CD74 |
| | S100A9, C3AR1, TAP2, IL1R2, IFNG, CTLA4, HP |
| | S100A9, C3AR1, TAP2, IL1R2, IFNG, CD74, HP |
| | S100A9, C3AR1, TAP2, IL1R2, CIITA, CD3D, CTLA4 |
| | S100A9, C3AR1, TAP2, IL1R2, CIITA, CD3D, CD74 |
| | S100A9, C3AR1, TAP2, IL1R2, CIITA, CD3D, HP |
| | S100A9, C3AR1, TAP2, IL1R2, CIITA, CTLA4, CD74 |
| | S100A9, C3AR1, TAP2, IL1R2, CIITA, CTLA4, HP |
| | S100A9, C3AR1, TAP2, IL1R2, CIITA, CD74, HP |
| | S100A9, C3AR1, TAP2, IL1R2, CD3D, CTLA4, CD74 |
| | S100A9, C3AR1, TAP2, IL1R2, CD3D, CTLA4, HP |
| | S100A9, C3AR1, TAP2, IL1R2, CD3D, CD74, HP |
| | S100A9, C3AR1, TAP2, IL1R2, CTLA4, CD74, HP |
| | S100A9, C3AR1, TAP2, IFNG, CIITA, CD3D, CTLA4 |
| | S100A9, C3AR1, TAP2, IFNG, CIITA, CD3D, CD74 |
| | S100A9, C3AR1, TAP2, IFNG, CIITA, CD3D, HP |
| | S100A9, C3AR1, TAP2, IFNG, CIITA, CTLA4, CD74 |
| | S100A9, C3AR1, TAP2, IFNG, CIITA, CTLA4, HP |
| | S100A9, C3AR1, TAP2, IFNG, CIITA, CD74, HP |
| | S100A9, C3AR1, TAP2, IFNG, CD3D, CTLA4, CD74 |
| | S100A9, C3AR1, TAP2, IFNG, CD3D, CTLA4, HP |
| | S100A9, C3AR1, TAP2, IFNG, CD3D, CD74, HP |
| | S100A9, C3AR1, TAP2, IFNG, CTLA4, CD74, HP |
| | S100A9, C3AR1, TAP2, CIITA, CD3D, CTLA4, CD74 |
| | S100A9, C3AR1, TAP2, CIITA, CD3D, CTLA4, HP |
| | S100A9, C3AR1, TAP2, CIITA, CD3D, CD74, HP |
| | S100A9, C3AR1, TAP2, CIITA, CTLA4, CD74, HP |
| | S100A9, C3AR1, TAP2, CD3D, CTLA4, CD74, HP |
| | S100A9, C3AR1, CX3CR1, IL1R2, IFNG, CIITA, CD3D |
| | S100A9, C3AR1, CX3CR1, IL1R2, IFNG, CIITA, CTLA4 |
| | S100A9, C3AR1, CX3CR1, IL1R2, IFNG, CIITA, CD74 |
| | S100A9, C3AR1, CX3CR1, IL1R2, IFNG, CIITA, HP |
| | S100A9, C3AR1, CX3CR1, IL1R2, IFNG, CD3D, CTLA4 |
| | S100A9, C3AR1, CX3CR1, IL1R2, IFNG, CD3D, CD74 |
| | S100A9, C3AR1, CX3CR1, IL1R2, IFNG, CD3D, HP |
| | S100A9, C3AR1, CX3CR1, IL1R2, IFNG, CTLA4, CD74 |
| | S100A9, C3AR1, CX3CR1, IL1R2, IFNG, CTLA4, HP |
| | S100A9, C3AR1, CX3CR1, IL1R2, IFNG, CD74, HP |
| | S100A9, C3AR1, CX3CR1, IL1R2, CIITA, CD3D, CTLA4 |
| | S100A9, C3AR1, CX3CR1, IL1R2, CIITA, CD3D, CD74 |
| | S100A9, C3AR1, CX3CR1, IL1R2, CIITA, CD3D, HP |
| | S100A9, C3AR1, CX3CR1, IL1R2, CIITA, CTLA4, CD74 |
| | S100A9, C3AR1, CX3CR1, IL1R2, CIITA, CTLA4, HP |
| | S100A9, C3AR1, CX3CR1, IL1R2, CIITA, CD74, HP |
| | S100A9, C3AR1, CX3CR1, IL1R2, CD3D, CTLA4, CD74 |
| | S100A9, C3AR1, CX3CR1, IL1R2, CD3D, CTLA4, HP |
| | S100A9, C3AR1, CX3CR1, IL1R2, CD3D, CD74, HP |
| | S100A9, C3AR1, CX3CR1, IL1R2, CTLA4, CD74, HP |
| | S100A9, C3AR1, CX3CR1, IFNG, CIITA, CD3D, CTLA4 |
| | S100A9, C3AR1, CX3CR1, IFNG, CIITA, CD3D, CD74 |
| | S100A9, C3AR1, CX3CR1, IFNG, CIITA, CD3D, HP |
| | S100A9, C3AR1, CX3CR1, IFNG, CIITA, CTLA4, CD74 |
| | S100A9, C3AR1, CX3CR1, IFNG, CIITA, CTLA4, HP |
| | S100A9, C3AR1, CX3CR1, IFNG, CIITA, CD74, HP |
| | S100A9, C3AR1, CX3CR1, IFNG, CD3D, CTLA4, CD74 |
| | S100A9, C3AR1, CX3CR1, IFNG, CD3D, CTLA4, HP |
| | S100A9, C3AR1, CX3CR1, IFNG, CD3D, CD74, HP |
| | S100A9, C3AR1, CX3CR1, IFNG, CTLA4, CD74, HP |
| | S100A9, C3AR1, CX3CR1, CIITA, CD3D, CTLA4, CD74 |
| | S100A9, C3AR1, CX3CR1, CHTA, CD3D, CTLA4, HP |
| | S100A9, C3AR1, CX3CR1, CIITA, CD3D, CD74, HP |
| | S100A9, C3AR1, CX3CR1, CIITA, CTLA4, CD74, HP |
| | S100A9, C3AR1, CX3CR1, CD3D, CTLA4, CD74, HP |
| | S100A9, C3AR1, IL1R2, IFNG, CIITA, CD3D, CTLA4 |
| | S100A9, C3AR1, IL1R2, IFNG, CIITA, CD3D, CD74 |
| | S100A9, C3AR1, IL1R2, IFNG, CIITA, CD3D, HP |
| | S100A9, C3AR1, IL1R2, IFNG, CIITA, CTLA4, CD74 |
| | S100A9, C3AR1, IL1R2, IFNG, CIITA, CTLA4, HP |
| | S100A9, C3AR1, IL1R2, IFNG, CIITA, CD74, HP |
| | S100A9, C3AR1, IL1R2, IFNG, CD3D, CTLA4, CD74 |
| | S100A9, C3AR1, IL1R2, IFNG, CD3D, CTLA4, HP |
| | S100A9, C3AR1, IL1R2, IFNG, CD3D, CD74, HP |
| | S100A9, C3AR1, IL1R2, IFNG, CTLA4, CD74, HP |
| | S100A9, C3AR1, IL1R2, CIITA, CD3D, CTLA4, CD74 |
| | S100A9, C3AR1, IL1R2, CIITA, CD3D, CTLA4, HP |
| | S100A9, C3AR1, IL1R2, CIITA, CD3D, CD74, HP |
| | S100A9, C3AR1, IL1R2, CIITA, CTLA4, CD74, HP |
| | S100A9, C3AR1, IL1R2, CD3D, CTLA4, CD74, HP |
| | S100A9, C3AR1, IFNG, CIITA, CD3D, CTLA4, CD74 |
| | S100A9, C3AR1, IFNG, CIITA, CD3D, CTLA4, HP |
| | S100A9, C3AR1, IFNG, CIITA, CD3D, CD74, HP |
| | S100A9, C3AR1, IFNG, CIITA, CTLA4, CD74, HP |
| | S100A9, C3AR1, IFNG, CD3D, CTLA4, CD74, HP |
| | S100A9, C3AR1, CIITA, CD3D, CTLA4, CD74, HP |
| | S100A9, TAP2, CX3CR1, IL1R2, IFNG, CIITA, CD3D |
| | S100A9, TAP2, CX3CR1, IL1R2, IFNG, CIITA, CTLA4 |
| | S100A9, TAP2, CX3CR1, IL1R2, IFNG, CIITA, CD74 |
| | S100A9, TAP2, CX3CR1, IL1R2, IFNG, CIITA, HP |
| | S100A9, TAP2, CX3CR1, IL1R2, IFNG, CD3D, CTLA4 |
| | S100A9, TAP2, CX3CR1, IL1R2, IFNG, CD3D, CD74 |
| | S100A9, TAP2, CX3CR1, IL1R2, IFNG, CD3D, HP |
| | S100A9, TAP2, CX3CR1, IL1R2, IFNG, CTLA4, CD74 |
| | S100A9, TAP2, CX3CR1, IL1R2, IFNG, CTLA4, HP |
| | S100A9, TAP2, CX3CR1, IL1R2, IFNG, CD74, HP |
| | S100A9, TAP2, CX3CR1, IL1R2, CIITA, CD3D, CTLA4 |
| | S100A9, TAP2, CX3CR1, IL1R2, CIITA, CD3D, CD74 |
| | S100A9, TAP2, CX3CR1, IL1R2, CIITA, CD3D, HP |
| | S100A9, TAP2, CX3CR1, IL1R2, CIITA, CTLA4, CD74 |
| | S100A9, TAP2, CX3CR1, IL1R2, CIITA, CTLA4, HP |
| | S100A9, TAP2, CX3CR1, IL1R2, CIITA, CD74, HP |
| | S100A9, TAP2, CX3CR1, IL1R2, CD3D, CTLA4, CD74 |
| | S100A9, TAP2, CX3CR1, IL1R2, CD3D, CTLA4, HP |
| | S100A9, TAP2, CX3CR1, IL1R2, CD3D, CD74, HP |
| | S100A9, TAP2, CX3CR1, IL1R2, CTLA4, CD74, HP |
| | S100A9, TAP2, CX3CR1, IFNG, CIITA, CD3D, CTLA4 |
| | S100A9, TAP2, CX3CR1, IFNG, CIITA, CD3D, CD74 |
| | S100A9, TAP2, CX3CR1, IFNG, CIITA, CD3D, HP |
| | S100A9, TAP2, CX3CR1, IFNG, CIITA, CTLA4, CD74 |
| | S100A9, TAP2, CX3CR1, IFNG, CIITA, CTLA4, HP |
| | S100A9, TAP2, CX3CR1, IFNG, CIITA, CD74, HP |
| | S100A9, TAP2, CX3CR1, IFNG, CD3D, CTLA4, CD74 |
| | S100A9, TAP2, CX3CR1, IFNG, CD3D, CTLA4, HP |
| | S100A9, TAP2, CX3CR1, IFNG, CD3D, CD74, HP |
| | S100A9, TAP2, CX3CR1, IFNG, CTLA4, CD74, HP |
| | S100A9, TAP2, CX3CR1, CIITA, CD3D, CTLA4, CD74 |
| | S100A9, TAP2, CX3CR1, CIITA, CD3D, CTLA4, HP |
| | S100A9, TAP2, CX3CR1, CIITA, CD3D, CD74, HP |
| | S100A9, TAP2, CX3CR1, CIITA, CTLA4, CD74, HP |
| | S100A9, TAP2, CX3CR1, CD3D, CTLA4, CD74, HP |
| | S100A9, TAP2, IL1R2, IFNG, CIITA, CD3D, CTLA4 |
| | S100A9, TAP2, IL1R2, IFNG, CIITA, CD3D, CD74 |
| | S100A9, TAP2, IL1R2, IFNG, CIITA, CD3D, HP |
| | S100A9, TAP2, IL1R2, IFNG, CIITA, CTLA4, CD74 |
| | S100A9, TAP2, IL1R2, IFNG, CIITA, CTLA4, HP |
| | S100A9, TAP2, IL1R2, IFNG, CIITA, CD74, HP |
| | S100A9, TAP2, IL1R2, IFNG, CD3D, CTLA4, CD74 |
| | S100A9, TAP2, IL1R2, IFNG, CD3D, CTLA4, HP |
| | S100A9, TAP2, IL1R2, IFNG, CD3D, CD74, HP |
| | S100A9, TAP2, IL1R2, IFNG, CTLA4, CD74, HP |
| | S100A9, TAP2, IL1R2, CIITA, CD3D, CTLA4, CD74 |
| | S100A9, TAP2, IL1R2, CIITA, CD3D, CTLA4, HP |
| | S100A9, TAP2, IL1R2, CIITA, CD3D, CD74, HP |
| | S100A9, TAP2, IL1R2, CIITA, CTLA4, CD74, HP |
| | S100A9, TAP2, IL1R2, CD3D, CTLA4, CD74, HP |
| | S100A9, TAP2, IFNG, CIITA, CD3D, CTLA4, CD74 |
| | S100A9, TAP2, IFNG, CIITA, CD3D, CTLA4, HP |
| | S100A9, TAP2, IFNG, CIITA, CD3D, CD74, HP |
| | S100A9, TAP2, IFNG, CIITA, CTLA4, CD74, HP |
| | S100A9, TAP2, IFNG, CD3D, CTLA4, CD74, HP |
| | S100A9, TAP2, CIITA, CD3D, CTLA4, CD74, HP |
| | S100A9, CX3CR1, IL1R2, IFNG, CIITA, CD3D, CTLA4 |
| | S100A9, CX3CR1, IL1R2, IFNG, CIITA, CD3D, CD74 |
| | S100A9, CX3CR1, IL1R2, IFNG, CIITA, CD3D, HP |
| | S100A9, CX3CR1, IL1R2, IFNG, CIITA, CTLA4, CD74 |
| | S100A9, CX3CR1, IL1R2, IFNG, CIITA, CTLA4, HP |
| | S100A9, CX3CR1, IL1R2, IFNG, CIITA, CD74, HP |
| | S100A9, CX3CR1, IL1R2, IFNG, CD3D, CTLA4, CD74 |
| | S100A9, CX3CR1, IL1R2, IFNG, CD3D, CTLA4, HP |
| | S100A9, CX3CR1, IL1R2, IFNG, CD3D, CD74, HP |
| | S100A9, CX3CR1, IL1R2, IFNG, CTLA4, CD74, HP |
| | S100A9, CX3CR1, IL1R2, CIITA, CD3D, CTLA4, CD74 |
| | S100A9, CX3CR1, IL1R2, CIITA, CD3D, CTLA4, HP |
| | S100A9, CX3CR1, IL1R2, CIITA, CD3D, CD74, HP |
| | S100A9, CX3CR1, IL1R2, CIITA, CTLA4, CD74, HP |
| | S100A9, CX3CR1, IL1R2, CD3D, CTLA4, CD74, HP |
| | S100A9, CX3CR1, IFNG, CIITA, CD3D, CTLA4, CD74 |
| | S100A9, CX3CR1, IFNG, CIITA, CD3D, CTLA4, HP |
| | S100A9, CX3CR1, IFNG, CIITA, CD3D, CD74, HP |
| | S100A9, CX3CR1, IFNG, CIITA, CTLA4, CD74, HP |
| | S100A9, CX3CR1, IFNG, CD3D, CTLA4, CD74, HP |
| | S100A9, CX3CR1, CIITA, CD3D, CTLA4, CD74, HP |
| | S100A9, IL1R2, IFNG, CIITA, CD3D, CTLA4, CD74 |
| | S100A9, IL1R2, IFNG, CIITA, CD3D, CTLA4, HP |
| | S100A9, IL1R2, IFNG, CIITA, CD3D, CD74, HP |
| | S100A9, IL1R2, IFNG, CIITA, CTLA4, CD74, HP |
| | S100A9, IL1R2, IFNG, CD3D, CTLA4, CD74, HP |
| | S100A9, IL1R2, CIITA, CD3D, CTLA4, CD74, HP |
| | S100A9, IFNG, CIITA, CD3D, CTLA4, CD74, HP |
| | C3AR1, TAP2, CX3CR1, IL1R2, IFNG, CIITA, CD3D |
| | C3AR1, TAP2, CX3CR1, IL1R2, IFNG, CIITA, CTLA4 |
| | C3AR1, TAP2, CX3CR1, IL1R2, IFNG, CIITA, CD74 |
| | C3AR1, TAP2, CX3CR1, IL1R2, IFNG, CIITA, HP |
| | C3AR1, TAP2, CX3CR1, IL1R2, IFNG, CD3D, CTLA4 |
| | C3AR1, TAP2, CX3CR1, IL1R2, IFNG, CD3D, CD74 |
| | C3AR1, TAP2, CX3CR1, IL1R2, IFNG, CD3D, HP |
| | C3AR1, TAP2, CX3CR1, IL1R2, IFNG, CTLA4, CD74 |
| | C3AR1, TAP2, CX3CR1, IL1R2, IFNG, CTLA4, HP |
| | C3AR1, TAP2, CX3CR1, IL1R2, IFNG, CD74, HP |
| | C3AR1, TAP2, CX3CR1, IL1R2, CIITA, CD3D, CTLA4 |
| | C3AR1, TAP2, CX3CR1, IL1R2, CIITA, CD3D, CD74 |
| | C3AR1, TAP2, CX3CR1, IL1R2, CIITA, CD3D, HP |
| | C3AR1, TAP2, CX3CR1, IL1R2, CIITA, CTLA4, CD74 |
| | C3AR1, TAP2, CX3CR1, IL1R2, CIITA, CTLA4, HP |
| | C3AR1, TAP2, CX3CR1, IL1R2, CIITA, CD74, HP |
| | C3AR1, TAP2, CX3CR1, IL1R2, CD3D, CTLA4, CD74 |
| | C3AR1, TAP2, CX3CR1, IL1R2, CD3D, CTLA4, HP |
| | C3AR1, TAP2, CX3CR1, IL1R2, CD3D, CD74, HP |
| | C3AR1, TAP2, CX3CR1, IL1R2, CTLA4, CD74, HP |
| | C3AR1, TAP2, CX3CR1, IFNG, CIITA, CD3D, CTLA4 |
| | C3AR1, TAP2, CX3CR1, IFNG, CIITA, CD3D, CD74 |
| | C3AR1, TAP2, CX3CR1, IFNG, CIITA, CD3D, HP |
| | C3AR1, TAP2, CX3CR1, IFNG, CIITA, CTLA4, CD74 |
| | C3AR1, TAP2, CX3CR1, IFNG, CIITA, CTLA4, HP |
| | C3AR1, TAP2, CX3CR1, IFNG, CIITA, CD74, HP |
| | C3AR1, TAP2, CX3CR1, IFNG, CD3D, CTLA4, CD74 |
| | C3AR1, TAP2, CX3CR1, IFNG, CD3D, CTLA4, HP |
| | C3AR1, TAP2, CX3CR1, IFNG, CD3D, CD74, HP |
| | C3AR1, TAP2, CX3CR1, IFNG, CTLA4, CD74, HP |
| | C3AR1, TAP2, CX3CR1, CIITA, CD3D, CTLA4, CD74 |
| | C3AR1, TAP2, CX3CR1, CIITA, CD3D, CTLA4, HP |
| | C3AR1, TAP2, CX3CR1, CIITA, CD3D, CD74, HP |
| | C3AR1, TAP2, CX3CR1, CIITA, CTLA4, CD74, HP |
| | C3AR1, TAP2, CX3CR1, CD3D, CTLA4, CD74, HP |
| | C3AR1, TAP2, IL1R2, IFNG, CIITA, CD3D, CTLA4 |
| | C3AR1, TAP2, IL1R2, IFNG, CIITA, CD3D, CD74 |
| | C3AR1, TAP2, IL1R2, IFNG, CIITA, CD3D, HP |
| | C3AR1, TAP2, IL1R2, IFNG, CIITA, CTLA4, CD74 |
| | C3AR1, TAP2, IL1R2, IFNG, CIITA, CTLA4, HP |
| | C3AR1, TAP2, IL1R2, IFNG, CIITA, CD74, HP |
| | C3AR1, TAP2, IL1R2, IFNG, CD3D, CTLA4, CD74 |
| | C3AR1, TAP2, IL1R2, IFNG, CD3D, CTLA4, HP |
| | C3AR1, TAP2, IL1R2, IFNG, CD3D, CD74, HP |
| | C3AR1, TAP2, IL1R2, IFNG, CTLA4, CD74, HP |
| | C3AR1, TAP2, IL1R2, CIITA, CD3D, CTLA4, CD74 |
| | C3AR1, TAP2, IL1R2, CIITA, CD3D, CTLA4, HP |
| | C3AR1, TAP2, IL1R2, CIITA, CD3D, CD74, HP |
| | C3AR1, TAP2, IL1R2, CIITA, CTLA4, CD74, HP |
| | C3AR1, TAP2, IL1R2, CD3D, CTLA4, CD74, HP |
| | C3AR1, TAP2, IFNG, CIITA, CD3D, CTLA4, CD74 |
| | C3AR1, TAP2, IFNG, CIITA, CD3D, CTLA4, HP |
| | C3AR1, TAP2, IFNG, CIITA, CD3D, CD74, HP |
| | C3AR1, TAP2, IFNG, CIITA, CTLA4, CD74, HP |
| | C3AR1, TAP2, IFNG, CD3D, CTLA4, CD74, HP |
| | C3AR1, TAP2, CIITA, CD3D, CTLA4, CD74, HP |
| | C3AR1, CX3CR1, IL1R2, IFNG, CIITA, CD3D, CTLA4 |
| | C3AR1, CX3CR1, IL1R2, IFNG, CIITA, CD3D, CD74 |
| | C3AR1, CX3CR1, IL1R2, IFNG, CIITA, CD3D, HP |
| | C3AR1, CX3CR1, IL1R2, IFNG, CIITA, CTLA4, CD74 |
| | C3AR1, CX3CR1, IL1R2, IFNG, CIITA, CTLA4, HP |
| | C3AR1, CX3CR1, IL1R2, IFNG, CIITA, CD74, HP |
| | C3AR1, CX3CR1, IL1R2, IFNG, CD3D, CTLA4, CD74 |
| | C3AR1, CX3CR1, IL1R2, IFNG, CD3D, CTLA4, HP |
| | C3AR1, CX3CR1, IL1R2, IFNG, CD3D, CD74, HP |
| | C3AR1, CX3CR1, IL1R2, IFNG, CTLA4, CD74, HP |
| | C3AR1, CX3CR1, IL1R2, CIITA, CD3D, CTLA4, CD74 |
| | C3AR1, CX3CR1, IL1R2, CIITA, CD3D, CTLA4, HP |
| | C3AR1, CX3CR1, IL1R2, CIITA, CD3D, CD74, HP |
| | C3AR1, CX3CR1, IL1R2, CIITA, CTLA4, CD74, HP |
| | C3AR1, CX3CR1, IL1R2, CD3D, CTLA4, CD74, HP |
| | C3AR1, CX3CR1, IFNG, CIITA, CD3D, CTLA4, CD74 |
| | C3AR1, CX3CR1, IFNG, CIITA, CD3D, CTLA4, HP |
| | C3AR1, CX3CR1, IFNG, CIITA, CD3D, CD74, HP |
| | C3AR1, CX3CR1, IFNG, CIITA, CTLA4, CD74, HP |
| | C3AR1, CX3CR1, IFNG, CD3D, CTLA4, CD74, HP |
| | C3AR1, CX3CR1, CIITA, CD3D, CTLA4, CD74, HP |
| | C3AR1, IL1R2, IFNG, CIITA, CD3D, CTLA4, CD74 |
| | C3AR1, IL1R2, IFNG, CIITA, CD3D, CTLA4, HP |
| | C3AR1, IL1R2, IFNG, CIITA, CD3D, CD74, HP |
| | C3AR1, IL1R2, IFNG, CIITA, CTLA4, CD74, HP |
| | C3AR1, IL1R2, IFNG, CD3D, CTLA4, CD74, HP |
| | C3AR1, IL1R2, CIITA, CD3D, CTLA4, CD74, HP |
| | C3AR1, IFNG, CIITA, CD3D, CTLA4, CD74, HP |
| | TAP2, CX3CR1, IL1R2, IFNG, CIITA, CD3D, CTLA4 |
| | TAP2, CX3CR1, IL1R2, IFNG, CIITA, CD3D, CD74 |
| | TAP2, CX3CR1, IL1R2, IFNG, CIITA, CD3D, HP |
| | TAP2, CX3CR1, IL1R2, IFNG, CIITA, CTLA4, CD74 |
| | TAP2, CX3CR1, IL1R2, IFNG, CIITA, CTLA4, HP |
| | TAP2, CX3CR1, IL1R2, IFNG, CIITA, CD74, HP |
| | TAP2, CX3CR1, IL1R2, IFNG, CD3D, CTLA4, CD74 |
| | TAP2, CX3CR1, IL1R2, IFNG, CD3D, CTLA4, HP |
| | TAP2, CX3CR1, IL1R2, IFNG, CD3D, CD74, HP |
| | TAP2, CX3CR1, IL1R2, IFNG, CTLA4, CD74, HP |
| | TAP2, CX3CR1, IL1R2, CIITA, CD3D, CTLA4, CD74 |
| | TAP2, CX3CR1, IL1R2, CIITA, CD3D, CTLA4, HP |
| | TAP2, CX3CR1, IL1R2, CIITA, CD3D, CD74, HP |
| | TAP2, CX3CR1, IL1R2, CIITA, CTLA4, CD74, HP |
| | TAP2, CX3CR1, IL1R2, CD3D, CTLA4, CD74, HP |
| | TAP2, CX3CR1, IFNG, CIITA, CD3D, CTLA4, CD74 |
| | TAP2, CX3CR1, IFNG, CIITA, CD3D, CTLA4, HP |
| | TAP2, CX3CR1, IFNG, CIITA, CD3D, CD74, HP |
| | TAP2, CX3CR1, IFNG, CIITA, CTLA4, CD74, HP |
| | TAP2, CX3CR1, IFNG, CD3D, CTLA4, CD74, HP |
| | TAP2, CX3CR1, CIITA, CD3D, CTLA4, CD74, HP |
| | TAP2, IL1R2, IFNG, CIITA, CD3D, CTLA4, CD74 |
| | TAP2, IL1R2, IFNG, CIITA, CD3D, CTLA4, HP |
| | TAP2, IL1R2, IFNG, CIITA, CD3D, CD74, HP |
| | TAP2, IL1R2, IFNG, CIITA, CTLA4, CD74, HP |
| | TAP2, IL1R2, IFNG, CD3D, CTLA4, CD74, HP |
| | TAP2, IL1R2, CIITA, CD3D, CTLA4, CD74, HP |
| | TAP2, IFNG, CIITA, CD3D, CTLA4, CD74, HP |
| | CX3CR1, IL1R2, IFNG, CIITA, CD3D, CTLA4, CD74 |
| | CX3CR1, IL1R2, IFNG, CIITA, CD3D, CTLA4, HP |
| | CX3CR1, IL1R2, IFNG, CIITA, CD3D, CD74, HP |
| | CX3CR1, IL1R2, IFNG, CIITA, CTLA4, CD74, HP |
| | CX3CR1, IL1R2, IFNG, CD3D, CTLA4, CD74, HP |
| | CX3CR1, IL1R2, CIITA, CD3D, CTLA4, CD74, HP |
| | CX3CR1, IFNG, CIITA, CD3D, CTLA4, CD74, HP |
| | IL1R2, IFNG, CIITA, CD3D, CTLA4, CD74, HP |
| Combinations of | S100A9, C3AR1, TAP2, CX3CR1, IL1R2, IFNG, CIITA, CD3D |
| eight genes | S100A9, C3AR1, TAP2, CX3CR1, IL1R2, IFNG, CIITA, CTLA4 |
| | S100A9, C3AR1, TAP2, CX3CR1, IL1R2, IFNG, CIITA, CD74 |
| | S100A9, C3AR1, TAP2, CX3CR1, IL1R2, IFNG, CIITA, HP |
| | S100A9, C3AR1, TAP2, CX3CR1, IL1R2, IFNG, CD3D, CTLA4 |
| | S100A9, C3AR1, TAP2, CX3CR1, IL1R2, IFNG, CD3D, CD74 |
| | S100A9, C3AR1, TAP2, CX3CR1, IL1R2, IFNG, CD3D, HP |
| | S100A9, C3AR1, TAP2, CX3CR1, IL1R2, IFNG, CTLA4, CD74 |
| | S100A9, C3AR1, TAP2, CX3CR1, IL1R2, IFNG, CTLA4, HP |
| | S100A9, C3AR1, TAP2, CX3CR1, IL1R2, IFNG, CD74, HP |
| | S100A9, C3AR1, TAP2, CX3CR1, IL1R2, CIITA, CD3D, CTLA4 |
| | S100A9, C3AR1, TAP2, CX3CR1, IL1R2, CIITA, CD3D, CD74 |
| | S100A9, C3AR1, TAP2, CX3CR1, IL1R2, CIITA, CD3D, HP |
| | S100A9, C3AR1, TAP2, CX3CR1, IL1R2, CIITA, CTLA4, CD74 |
| | S100A9, C3AR1, TAP2, CX3CR1, IL1R2, CIITA, CTLA4, HP |
| | S100A9, C3AR1, TAP2, CX3CR1, IL1R2, CIITA, CD74, HP |
| | S100A9, C3AR1, TAP2, CX3CR1, IL1R2, CD3D, CTLA4, CD74 |
| | S100A9, C3AR1, TAP2, CX3CR1, IL1R2, CD3D, CTLA4, HP |
| | S100A9, C3AR1, TAP2, CX3CR1, IL1R2, CD3D, CD74, HP |
| | S100A9, C3AR1, TAP2, CX3CR1, IL1R2, CTLA4, CD74, HP |
| | S100A9, C3AR1, TAP2, CX3CR1, IFNG, CIITA, CD3D, CTLA4 |
| | S100A9, C3AR1, TAP2, CX3CR1, IFNG, CIITA, CD3D, CD74 |
| | S100A9, C3AR1, TAP2, CX3CR1, IFNG, CIITA, CD3D, HP |
| | S100A9, C3AR1, TAP2, CX3CR1, IFNG, CIITA, CTLA4, CD74 |
| | S100A9, C3AR1, TAP2, CX3CR1, IFNG, CIITA, CTLA4, HP |
| | S100A9, C3AR1, TAP2, CX3CR1, IFNG, CIITA, CD74, HP |
| | S100A9, C3AR1, TAP2, CX3CR1, IFNG, CD3D, CTLA4, CD74 |
| | S100A9, C3AR1, TAP2, CX3CR1, IFNG, CD3D, CTLA4, HP |
| | S100A9, C3AR1, TAP2, CX3CR1, IFNG, CD3D, CD74, HP |
| | S100A9, C3AR1, TAP2, CX3CR1, IFNG, CTLA4, CD74, HP |
| | S100A9, C3AR1, TAP2, CX3CR1, CIITA, CD3D, CTLA4, CD74 |
| | S100A9, C3AR1, TAP2, CX3CR1, CIITA, CD3D, CTLA4, HP |
| | S100A9, C3AR1, TAP2, CX3CR1, CIITA, CD3D, CD74, HP |
| | S100A9, C3AR1, TAP2, CX3CR1, CIITA, CTLA4, CD74, HP |
| | S100A9, C3AR1, TAP2, CX3CR1, CD3D, CTLA4, CD74, HP |
| | S100A9, C3AR1, TAP2, IL1R2, IFNG, CIITA, CD3D, CTLA4 |
| | S100A9, C3AR1, TAP2, IL1R2, IFNG, CIITA, CD3D, CD74 |
| | S100A9, C3AR1, TAP2, IL1R2, IFNG, CIITA, CD3D, HP |
| | S100A9, C3AR1, TAP2, IL1R2, IFNG, CIITA, CTLA4, CD74 |
| | S100A9, C3AR1, TAP2, IL1R2, IFNG, CIITA, CTLA4, HP |
| | S100A9, C3AR1, TAP2, IL1R2, IFNG, CIITA, CD74, HP |
| | S100A9, C3AR1, TAP2, IL1R2, IFNG, CD3D, CTLA4, CD74 |
| | S100A9, C3AR1, TAP2, IL1R2, IFNG, CD3D, CTLA4, HP |
| | S100A9, C3AR1, TAP2, IL1R2, IFNG, CD3D, CD74, HP |
| | S100A9, C3AR1, TAP2, IL1R2, IFNG, CTLA4, CD74, HP |
| | S100A9, C3AR1, TAP2, IL1R2, CIITA, CD3D, CTLA4, CD74 |
| | S100A9, C3AR1, TAP2, IL1R2, CIITA, CD3D, CTLA4, HP |
| | S100A9, C3AR1, TAP2, IL1R2, CIITA, CD3D, CD74, HP |
| | S100A9, C3AR1, TAP2, IL1R2, CIITA, CTLA4, CD74, HP |
| | S100A9, C3AR1, TAP2, IL1R2, CD3D, CTLA4, CD74, HP |
| | S100A9, C3AR1, TAP2, IFNG, CIITA, CD3D, CTLA4, CD74 |
| | S100A9, C3AR1, TAP2, IFNG, CIITA, CD3D, CTLA4, HP |
| | S100A9, C3AR1, TAP2, IFNG, CIITA, CD3D, CD74, HP |
| | S100A9, C3AR1, TAP2, IFNG, CIITA, CTLA4, CD74, HP |
| | S100A9, C3AR1, TAP2, IFNG, CD3D, CTLA4, CD74, HP |
| | S100A9, C3AR1, TAP2, CIITA, CD3D, CTLA4, CD74, HP |
| | S100A9, C3AR1, CX3CR1, IL1R2, IFNG, CIITA, CD3D, CTLA4 |
| | S100A9, C3AR1, CX3CR1, IL1R2, IFNG, CIITA, CD3D, CD74 |
| | S100A9, C3AR1, CX3CR1, IL1R2, IFNG, CIITA, CD3D, HP |
| | S100A9, C3AR1, CX3CR1, IL1R2, IFNG, CIITA, CTLA4, CD74 |
| | S100A9, C3AR1, CX3CR1, IL1R2, IFNG, CIITA, CTLA4, HP |
| | S100A9, C3AR1, CX3CR1, IL1R2, IFNG, CIITA, CD74, HP |
| | S100A9, C3AR1, CX3CR1, IL1R2, IFNG, CD3D, CTLA4, CD74 |
| | S100A9, C3AR1, CX3CR1, IL1R2, IFNG, CD3D, CTLA4, HP |
| | S100A9, C3AR1, CX3CR1, IL1R2, IFNG, CD3D, CD74, HP |
| | S100A9, C3AR1, CX3CR1, IL1R2, IFNG, CTLA4, CD74, HP |
| | S100A9, C3AR1, CX3CR1, IL1R2, CIITA, CD3D, CTLA4, CD74 |
| | S100A9, C3AR1, CX3CR1, IL1R2, CIITA, CD3D, CTLA4, HP |
| | S100A9, C3AR1, CX3CR1, IL1R2, CIITA, CD3D, CD74, HP |
| | S100A9, C3AR1, CX3CR1, IL1R2, CIITA, CTLA4, CD74, HP |
| | S100A9, C3AR1, CX3CR1, IL1R2, CD3D, CTLA4, CD74, HP |
| | S100A9, C3AR1, CX3CR1, IFNG, CIITA, CD3D, CTLA4, CD74 |
| | S100A9, C3AR1, CX3CR1, IFNG, CIITA, CD3D, CTLA4, HP |
| | S100A9, C3AR1, CX3CR1, IFNG, CIITA, CD3D, CD74, HP |
| | S100A9, C3AR1, CX3CR1, IFNG, CIITA, CTLA4, CD74, HP |
| | S100A9, C3AR1, CX3CR1, IFNG, CD3D, CTLA4, CD74, HP |
| | S100A9, C3AR1, CX3CR1, CIITA, CD3D, CTLA4, CD74, HP |
| | S100A9, C3AR1, IL1R2, IFNG, CIITA, CD3D, CTLA4, CD74 |
| | S100A9, C3AR1, IL1R2, IFNG, CIITA, CD3D, CTLA4, HP |
| | S100A9, C3AR1, IL1R2, IFNG, CIITA, CD3D, CD74, HP |
| | S100A9, C3AR1, IL1R2, IFNG, CIITA, CTLA4, CD74, HP |
| | S100A9, C3AR1, IL1R2, IFNG, CD3D, CTLA4, CD74, HP |
| | S100A9, C3AR1, IL1R2, CIITA, CD3D, CTLA4, CD74, HP |
| | S100A9, C3AR1, IFNG, CIITA, CD3D, CTLA4, CD74, HP |
| | S100A9, TAP2, CX3CR1, IL1R2, IFNG, CIITA, CD3D, CTLA4 |
| | S100A9, TAP2, CX3CR1, IL1R2, IFNG, CIITA, CD3D, CD74 |
| | S100A9, TAP2, CX3CR1, IL1R2, IFNG, CIITA, CD3D, HP |
| | S100A9, TAP2, CX3CR1, IL1R2, IFNG, CIITA, CTLA4, CD74 |
| | S100A9, TAP2, CX3CR1, IL1R2, IFNG, CIITA, CTLA4, HP |
| | S100A9, TAP2, CX3CR1, IL1R2, IFNG, CIITA, CD74, HP |
| | S100A9, TAP2, CX3CR1, IL1R2, IFNG, CD3D, CTLA4, CD74 |
| | S100A9, TAP2, CX3CR1, IL1R2, IFNG, CD3D, CTLA4, HP |
| | S100A9, TAP2, CX3CR1, IL1R2, IFNG, CD3D, CD74, HP |
| | S100A9, TAP2, CX3CR1, IL1R2, IFNG, CTLA4, CD74, HP |
| | S100A9, TAP2, CX3CR1, IL1R2, CIITA, CD3D, CTLA4, CD74 |
| | S100A9, TAP2, CX3CR1, IL1R2, CIITA, CD3D, CTLA4, HP |
| | S100A9, TAP2, CX3CR1, IL1R2, CIITA, CD3D, CD74, HP |
| | S100A9, TAP2, CX3CR1, IL1R2, CIITA, CTLA4, CD74, HP |
| | S100A9, TAP2, CX3CR1, IL1R2, CD3D, CTLA4, CD74, HP |
| | S100A9, TAP2, CX3CR1, IFNG, CIITA, CD3D, CTLA4, CD74 |
| | S100A9, TAP2, CX3CR1, IFNG, CIITA, CD3D, CTLA4, HP |
| | S100A9, TAP2, CX3CR1, IFNG, CIITA, CD3D, CD74, HP |
| | S100A9, TAP2, CX3CR1, IFNG, CIITA, CTLA4, CD74, HP |
| | S100A9, TAP2, CX3CR1, IFNG, CD3D, CTLA4, CD74, HP |
| | S100A9, TAP2, CX3CR1, CIITA, CD3D, CTLA4, CD74, HP |
| | S100A9, TAP2, IL1R2, IFNG, CIITA, CD3D, CTLA4, CD74 |
| | S100A9, TAP2, IL1R2, IFNG, CIITA, CD3D, CTLA4, HP |
| | S100A9, TAP2, IL1R2, IFNG, CIITA, CD3D, CD74, HP |
| | S100A9, TAP2, IL1R2, IFNG, CIITA, CTLA4, CD74, HP |
| | S100A9, TAP2, IL1R2, IFNG, CD3D, CTLA4, CD74, HP |
| | S100A9, TAP2, IL1R2, CIITA, CD3D, CTLA4, CD74, HP |
| | S100A9, TAP2, IFNG, CIITA, CD3D, CTLA4, CD74, HP |
| | S100A9, CX3CR1, IL1R2, IFNG, CIITA, CD3D, CTLA4, CD74 |
| | S100A9, CX3CR1, IL1R2, IFNG, CIITA, CD3D, CTLA4, HP |
| | S100A9, CX3CR1, IL1R2, IFNG, CIITA, CD3D, CD74, HP |
| | S100A9, CX3CR1, IL1R2, IFNG, CIITA, CTLA4, CD74, HP |
| | S100A9, CX3CR1, IL1R2, IFNG, CD3D, CTLA4, CD74, HP |
| | S100A9, CX3CR1, IL1R2, CIITA, CD3D, CTLA4, CD74, HP |
| | S100A9, CX3CR1, IFNG, CIITA, CD3D, CTLA4, CD74, HP |
| | S100A9, IL1R2, IFNG, CIITA, CD3D, CTLA4, CD74, HP |
| | C3AR1, TAP2, CX3CR1, IL1R2, IFNG, CIITA, CD3D, CTLA4 |
| | C3AR1, TAP2, CX3CR1, IL1R2, IFNG, CIITA, CD3D, CD74 |
| | C3AR1, TAP2, CX3CR1, IL1R2, IFNG, CIITA, CD3D, HP |
| | C3AR1, TAP2, CX3CR1, IL1R2, IFNG, CIITA, CTLA4, CD74 |
| | C3AR1, TAP2, CX3CR1, IL1R2, IFNG, CIITA, CTLA4, HP |
| | C3AR1, TAP2, CX3CR1, IL1R2, IFNG, CIITA, CD74, HP |
| | C3AR1, TAP2, CX3CR1, IL1R2, IFNG, CD3D, CTLA4, CD74 |
| | C3AR1, TAP2, CX3CR1, IL1R2, IFNG, CD3D, CTLA4, HP |
| | C3AR1, TAP2, CX3CR1, IL1R2, IFNG, CD3D, CD74, HP |
| | C3AR1, TAP2, CX3CR1, IL1R2, IFNG, CTLA4, CD74, HP |
| | C3AR1, TAP2, CX3CR1, IL1R2, CIITA, CD3D, CTLA4, CD74 |
| | C3AR1, TAP2, CX3CR1, IL1R2, CIITA, CD3D, CTLA4, HP |
| | C3AR1, TAP2, CX3CR1, IL1R2, CIITA, CD3D, CD74, HP |
| | C3AR1, TAP2, CX3CR1, IL1R2, CIITA, CTLA4, CD74, HP |
| | C3AR1, TAP2, CX3CR1, IL1R2, CD3D, CTLA4, CD74, HP |
| | C3AR1, TAP2, CX3CR1, IFNG, CIITA, CD3D, CTLA4, CD74 |
| | C3AR1, TAP2, CX3CR1, IFNG, CIITA, CD3D, CTLA4, HP |
| | C3AR1, TAP2, CX3CR1, IFNG, CIITA, CD3D, CD74, HP |
| | C3AR1, TAP2, CX3CR1, IFNG, CIITA, CTLA4, CD74, HP |
| | C3AR1, TAP2, CX3CR1, IFNG, CD3D, CTLA4, CD74, HP |
| | C3AR1, TAP2, CX3CR1, CIITA, CD3D, CTLA4, CD74, HP |
| | C3AR1, TAP2, IL1R2, IFNG, CIITA, CD3D, CTLA4, CD74 |
| | C3AR1, TAP2, IL1R2, IFNG, CIITA, CD3D, CTLA4, HP |
| | C3AR1, TAP2, IL1R2, IFNG, CIITA, CD3D, CD74, HP |
| | C3AR1, TAP2, IL1R2, IFNG, CIITA, CTLA4, CD74, HP |
| | C3AR1, TAP2, IL1R2, IFNG, CD3D, CTLA4, CD74, HP |
| | C3AR1, TAP2, IL1R2, CIITA, CD3D, CTLA4, CD74, HP |
| | C3AR1, TAP2, IFNG, CIITA, CD3D, CTLA4, CD74, HP |
| | C3AR1, CX3CR1, IL1R2, IFNG, CIITA, CD3D, CTLA4, CD74 |
| | C3AR1, CX3CR1, IL1R2, IFNG, CIITA, CD3D, CTLA4, HP |
| | C3AR1, CX3CR1, IL1R2, IFNG, CIITA, CD3D, CD74, HP |
| | C3AR1, CX3CR1, IL1R2, IFNG, CIITA, CTLA4, CD74, HP |
| | C3AR1, CX3CR1, IL1R2, IFNG, CD3D, CTLA4, CD74, HP |
| | C3AR1, CX3CR1, IL1R2, CIITA, CD3D, CTLA4, CD74, HP |
| | C3AR1, CX3CR1, IFNG, CIITA, CD3D, CTLA4, CD74, HP |
| | C3AR1, IL1R2, IFNG, CIITA, CD3D, CTLA4, CD74, HP |
| | TAP2, CX3CR1, IL1R2, IFNG, CIITA, CD3D, CTLA4, CD74 |
| | TAP2, CX3CR1, IL1R2, IFNG, CIITA, CD3D, CTLA4, HP |
| | TAP2, CX3CR1, IL1R2, IFNG, CIITA, CD3D, CD74, HP |
| | TAP2, CX3CR1, IL1R2, IFNG, CIITA, CTLA4, CD74, HP |
| | TAP2, CX3CR1, IL1R2, IFNG, CD3D, CTLA4, CD74, HP |
| | TAP2, CX3CR1, IL1R2, CIITA, CD3D, CTLA4, CD74, HP |
| | TAP2, CX3CR1, IFNG, CIITA, CD3D, CTLA4, CD74, HP |
| | TAP2, IL1R2, IFNG, CIITA, CD3D, CTLA4, CD74, HP |
| | CX3CR1, IL1R2, IFNG, CIITA, CD3D, CTLA4, CD74, HP |
| Combinations of | S100A9, C3AR1, TAP2, CX3CR1, IL1R2, IFNG, CIITA, CD3D, CTLA4 |
| nine genes | S100A9, C3AR1, TAP2, CX3CR1, IL1R2, IFNG, CIITA, CD3D, CD74 |
| | S100A9, C3AR1, TAP2, CX3CR1, IL1R2, IFNG, CIITA, CD3D, HP |
| | S100A9, C3AR1, TAP2, CX3CR1, IL1R2, IFNG, CIITA, CTLA4, CD74 |
| | S100A9, C3AR1, TAP2, CX3CR1, IL1R2, IFNG, CIITA, CTLA4, HP |
| | S100A9, C3AR1, TAP2, CX3CR1, IL1R2, IFNG, CIITA, CD74, HP |
| | S100A9, C3AR1, TAP2, CX3CR1, IL1R2, IFNG, CD3D, CTLA4, CD74 |
| | S100A9, C3AR1, TAP2, CX3CR1, IL1R2, IFNG, CD3D, CTLA4, HP |
| | S100A9, C3AR1, TAP2, CX3CR1, IL1R2, IFNG, CD3D, CD74, HP |
| | S100A9, C3AR1, TAP2, CX3CR1, IL1R2, IFNG, CTLA4, CD74, HP |
| | S100A9, C3AR1, TAP2, CX3CR1, IL1R2, CIITA, CD3D, CTLA4, CD74 |
| | S100A9, C3AR1, TAP2, CX3CR1, IL1R2, CIITA, CD3D, CTLA4, HP |
| | S100A9, C3AR1, TAP2, CX3CR1, IL1R2, CIITA, CD3D, CD74, HP |
| | S100A9, C3AR1, TAP2, CX3CR1, IL1R2, CIITA, CTLA4, CD74, HP |
| | S100A9, C3AR1, TAP2, CX3CR1, IL1R2, CD3D, CTLA4, CD74, HP |
| | S100A9, C3AR1, TAP2, CX3CR1, IFNG, CIITA, CD3D, CTLA4, CD74 |
| | S100A9, C3AR1, TAP2, CX3CR1, IFNG, CIITA, CD3D, CTLA4, HP |
| | S100A9, C3AR1, TAP2, CX3CR1, IFNG, CIITA, CD3D, CD74, HP |
| | S100A9, C3AR1, TAP2, CX3CR1, IFNG, CIITA, CTLA4, CD74, HP |
| | S100A9, C3AR1, TAP2, CX3CR1, IFNG, CD3D, CTLA4, CD74, HP |
| | S100A9, C3AR1, TAP2, CX3CR1, CIITA, CD3D, CTLA4, CD74, HP |
| | S100A9, C3AR1, TAP2, IL1R2, IFNG, CIITA, CD3D, CTLA4, CD74 |
| | S100A9, C3AR1, TAP2, IL1R2, IFNG, CIITA, CD3D, CTLA4, HP |
| | S100A9, C3AR1, TAP2, IL1R2, IFNG, CIITA, CD3D, CD74, HP |
| | S100A9, C3AR1, TAP2, IL1R2, IFNG, CIITA, CTLA4, CD74, HP |
| | S100A9, C3AR1, TAP2, IL1R2, IFNG, CD3D, CTLA4, CD74, HP |
| | S100A9, C3AR1, TAP2, IL1R2, CIITA, CD3D, CTLA4, CD74, HP |
| | S100A9, C3AR1, TAP2, IFNG, CIITA, CD3D, CTLA4, CD74, HP |
| | S100A9, C3AR1, CX3CR1, IL1R2, IFNG, CIITA, CD3D, CTLA4, CD74 |
| | S100A9, C3AR1, CX3CR1, IL1R2, IFNG, CIITA, CD3D, CTLA4, HP |
| | S100A9, C3AR1, CX3CR1, IL1R2, IFNG, CIITA, CD3D, CD74, HP |
| | S100A9, C3AR1, CX3CR1, IL1R2, IFNG, CIITA, CTLA4, CD74, HP |
| | S100A9, C3AR1, CX3CR1, IL1R2, IFNG, CD3D, CTLA4, CD74, HP |
| | S100A9, C3AR1, CX3CR1, IL1R2, CIITA, CD3D, CTLA4, CD74, HP |
| | S100A9, C3AR1, CX3CR1, IFNG, CIITA, CD3D, CTLA4, CD74, HP |
| | S100A9, C3AR1, IL1R2, IFNG, CIITA, CD3D, CTLA4, CD74, HP |
| | S100A9, TAP2, CX3CR1, IL1R2, IFNG, CIITA, CD3D, CTLA4, CD74 |
| | S100A9, TAP2, CX3CR1, IL1R2, IFNG, CIITA, CD3D, CTLA4, HP |
| | S100A9, TAP2, CX3CR1, IL1R2, IFNG, CIITA, CD3D, CD74, HP |
| | S100A9, TAP2, CX3CR1, IL1R2, IFNG, CIITA, CTLA4, CD74, HP |
| | S100A9, TAP2, CX3CR1, IL1R2, IFNG, CD3D, CTLA4, CD74, HP |
| | S100A9, TAP2, CX3CR1, IL1R2, CIITA, CD3D, CTLA4, CD74, HP |
| | S100A9, TAP2, CX3CR1, IFNG, CIITA, CD3D, CTLA4, CD74, HP |
| | S100A9, TAP2, IL1R2, IFNG, CIITA, CD3D, CTLA4, CD74, HP |
| | S100A9, CX3CR1, IL1R2, IFNG, CIITA, CD3D, CTLA4, CD74, HP |
| | C3AR1, TAP2, CX3CR1, IL1R2, IFNG, CIITA, CD3D, CTLA4, CD74 |
| | C3AR1, TAP2, CX3CR1, IL1R2, IFNG, CIITA, CD3D, CTLA4, HP |
| | C3AR1, TAP2, CX3CR1, IL1R2, IFNG, CIITA, CD3D, CD74, HP |
| | C3AR1, TAP2, CX3CR1, IL1R2, IFNG, CIITA, CTLA4, CD74, HP |
| | C3AR1, TAP2, CX3CR1, IL1R2, IFNG, CD3D, CTLA4, CD74, HP |
| | C3AR1, TAP2, CX3CR1, IL1R2, CIITA, CD3D, CTLA4, CD74, HP |
| | C3AR1, TAP2, CX3CR1, IFNG, CIITA, CD3D, CTLA4, CD74, HP |
| | C3AR1, TAP2, IL1R2, IFNG, CIITA, CD3D, CTLA4, CD74, HP |
| | C3AR1, CX3CR1, IL1R2, IFNG, CIITA, CD3D, CTLA4, CD74, HP |
| | TAP2, CX3CR1, IL1R2, IFNG, CIITA, CD3D, CTLA4, CD74, HP |
| Combinations of | S100A9, C3AR1, TAP2, CX3CR1, IL1R2, IFNG, CIITA, CD3D, CTLA4, CD74 |
| ten genes | S100A9, C3AR1, TAP2, CX3CR1, IL1R2, IFNG, CIITA, CD3D, CTLA4, HP |
| | S100A9, C3AR1, TAP2, CX3CR1, IL1R2, IFNG, CIITA, CD3D, CD74, HP |
| | S100A9, C3AR1, TAP2, CX3CR1, IL1R2, IFNG, CIITA, CTLA4, CD74, HP |
| | S100A9, C3AR1, TAP2, CX3CR1, IL1R2, IFNG, CD3D, CTLA4, CD74, HP |
| | S100A9, C3AR1, TAP2, CX3CR1, IL1R2, CIITA, CD3D, CTLA4, CD74, HP |
| | S100A9, C3AR1, TAP2, CX3CR1, IFNG, CIITA, CD3D, CTLA4, CD74, HP |
| | S100A9, C3AR1, TAP2, IL1R2, IFNG, CIITA, CD3D, CTLA4, CD74, HP |
| | S100A9, C3AR1, CX3CR1, IL1R2, IFNG, CIITA, CD3D, CTLA4, CD74, HP |
| | S100A9, TAP2, CX3CR1, IL1R2, IFNG, CIITA, CD3D, CTLA4, CD74, HP |
| | C3AR1, TAP2, CX3CR1, IL1R2, IFNG, CIITA, CD3D, CTLA4, CD74, HP |
| Combinations of | S100A9, C3AR1, TAP2, CX3CR1, IL1R2, IFNG, CIITA, CD3D, CTLA4, |
| eleven genes | CD74, HP |

Table 3 List of preferred combinations of 1 to 11 other gene(s), the expression of which can be measured in combination with the expression of CD177.

Preferably, in the method as described previously, in all the embodiments thereof, the biological sample is a blood sample, preferably a blood sample or a blood-derived sample (e.g. PBMCs which can be obtained using the Ficoll method, well known to those skilled in the art, or purified monocytes).

The measurement of the expression (or expression level) of a gene consists in quantifying at least one expression product of the gene. For the purposes of the present invention, the expression product of a gene is any biological molecule resulting from the expression of said gene. More particularly, the expression product of the gene may be an RNA transcript. “Transcript” means RNA, and in particular messenger RNA (mRNA) resulting from the transcription of the gene. More specifically, the transcripts are RNAs produced by the transcription of a gene followed by post-transcriptional modifications of the pre-RNA forms. In the context of the present invention, the expression level of one or more RNA transcripts of the same gene can be measured. Thus, preferably, in the method as described previously, in all the embodiments thereof, the expression of the gene(s) (i.e. the expression of CD177, and optionally of one or more other genes of interest, as described previously) is measured at the RNA or mRNA transcript level. In the case of an mRNA transcript, the detection may be performed by a direct method, by any process known to those skilled in the art which makes it possible to determine the presence of said transcript in the sample, or by indirect detection of the transcript after conversion of the latter into DNA, or after amplification of said transcript or after amplification of the DNA obtained after conversion of said transcript into DNA. Numerous methods exist for the detection of nucleic acids (see for example Kricka et al., Clinical Chemistry, 1999, no. 45(4), p. 453-458; Relier G. H. et al., DNA Probes, 2nd Ed., Stockton Press, 1993, sections 5 and 6, p. 173-249). The expression of the genes may particularly be measured by Reverse Transcription-Polymerase Chain Reaction or RT-PCR, preferably by quantitative RT-PCR or RT-qPCR (for example using the FilmArray® technology or the Biomark™ platform from Fluidigm), by sequencing (preferably by high-throughput sequencing) or by hybridization techniques (for example with hybridization microarrays or by techniques of the NanoString® nCounter® type).

The measurement of the expression level of a gene makes it possible in particular to determine the quantity of one or more transcripts present in the biological sample or to give a value derived therefrom. A value derived from the quantity may for example be the absolute concentration, calculated by virtue of a calibration curve obtained from successive dilutions of a solution of amplicons having a given concentration. It may also correspond to the value of the normalized and calibrated quantity, such as the CNRQ (Calibrated Normalized Relative Quantity, (Hellemans et al (2007), Genome biology 8(2):R19), which integrates the values of a reference sample (or of a calibrator) and of one or more housekeeping genes (also referred to as reference genes). By way of example of housekeeping genes, mentioned may be made of the genes DECR1, HPRT1, PPIB, RPLP0, PPIA, GLYR1, RANBP3, 18S, GAPDH and ACTB.

Thus, preferably, in the method as described previously, in all the embodiments thereof, the expression of the gene(s) of interest is normalized in relation to the expression of one or more housekeeping genes (or reference genes), as is known to those skilled in the art; more preferably still, using one or more of the following housekeeping genes: DECR1 (chromosomal location of the gene according to GRCh38/hg38: chr8:90,001,352-90,053,633), HPRT1 (chromosomal location of the gene according to GRCh38/hg38: chrX:134,452,842-134,520,513) and PPIB (chromosomal location of the gene according to GRCh38/hg38: chr15:64,155,812-64,163,205).

Preferably, in the method as described previously, in all the embodiments thereof, the expression of the gene(s) of interest (preferably the normalized expression) in the biological sample from the patient is compared to a reference value or to the expression of the same gene(s) of interest (preferably the normalized expression) in a reference biological sample (this data being used to calculate the CNRQ, as mentioned above). The reference sample may for example be a sample originating from a volunteer (healthy individual), from a patient, or from a mixture of samples from several volunteers (on the one hand) or from several patients (on the other hand). The reference sample can also be a sample taken from a volunteer (or a mixture of samples taken from several volunteers) then treated ex vivo by an immune system-stimulating agent (such as LPS or lipopolysaccharide). The reference sample can also be a mixture of untreated sample(s) and sample(s) treated ex vivo by an immune system-stimulating agent.

Preferably, the method for determining the risk of incidence of a healthcare-associated infection, as described previously, in all the embodiments thereof, also comprises a step of managing the healthcare provisions in order to reduce the risk of incidence of a healthcare-associated infection. A patient identified as having an increased risk of incidence of a healthcare-associated infection may have adapted management of healthcare provisions with the aim of reducing the risk of incidence of a healthcare-associated infection and, for example, in order to reduce the risk of developing sepsis, septic shock or even the risk of death. By way of examples of healthcare provision management, mention may be made of an immunomodulatory treatment adapted to the patient, or else a prophylactic antibiotic treatment, it being possible for the two treatments to be combined, and/or admittance to an ongoing care unit or resuscitation unit in order to reduce the risk of incidence of a healthcare-associated infection, for example to reduce the risk of developing sepsis, septic shock or even the risk of death, in the days following the measurement of the expression of the biomarker(s). Preferably, the immunomodulatory treatment is an immunostimulant treatment if it is determined that the individual is immunosuppressed, or an anti-inflammatory treatment if it is determined that the individual has an inflammatory state. Among the immunostimulant treatments which may be selected, mention may for example be made of the group of the interleukins, in particular IL-7, IL-15 or IL-3, growth factors, in particular GM-CSF, interferons, in particular IFNγ, Toll agonists, antibodies, in particular anti-PD1, anti-PDL1, anti-LAG3, anti-TIM3, anti-IL-10 or anti-CTLA4 antibodies transferrins and apoptosis inhibitor molecules, FLT3L, Thymosin al, adrenergic antagonists. Among the anti-inflammatory treatments, mention may particularly be made of the group of glucocorticoids, cytostatic agents, molecules acting on immunophilins and cytokines, molecules which block the IL-1 receptor and anti-TNF treatments. Examples of prophylactic antibiotic treatments suitable for preventing pneumonia are described in particular in Annales Françaises d'Anesthésie et de Réanimation [French Annals of Anaesthesia and Resuscitation] (30; 2011; 168-190). Conversely, a patient not at risk of incidence of a healthcare-associated infection could be quickly admitted to an outpatient hospital service, for example an infectious diseases unit, rather than remaining in a closely-monitored unit which they do not need.

Another subject of the invention is a kit comprising means for amplifying (e.g. primers) and/or means for detecting (e.g. probes) the expression of CD177 and of one or more other gene(s), as indicated previously, in all the embodiments, and particularly preferably one, two, three, four, five, six, seven, eight, nine, ten or eleven genes selected from the group consisting of: CD74, CIITA, IFNG, IL1R2, C3AR1, TAP2, HP, CX3R1, S100A9, CTLA4 and CD3D); said kit being characterized in that all the amplification and/or detection means of said kit enable the detection and/or amplification of at most 100, 90, 80, 70, 60, 50, 40, 30, 29, 28, 27, 26, 25, 24, 23, 22, 21, 20, 19, 18, 17, 16, 15, 14, 13, 12, 11, 10, 9, 8, 7, 6, 5, 4, 3, 2 biomarkers in total. “Biomarker” (or “marker”) means a biological characteristic which can be objectively measured and which is indicative of normal or pathological biological processes or of a pharmacological response to a therapeutic intervention. This biomarker may in particular be detectable at the mRNA level. More particularly, the biomarker may be an endogenous biomarker or loci (such as a gene or an HERV/Human Endogenous RetroVirus, found in an individual's chromosomal material) or an exogenous biomarker (such as a virus).

Thus, said kit may for example also comprise means for amplifying and/or detecting one or more housekeeping genes (preferably selected from the list consisting of: DECR1, HPRT1 and PPIB). The kit may also comprise positive control means, making it possible to qualify the quality of RNA extraction, the quality of any amplification and/or hybridization process.

“Primer” or “amplification primer” means a nucleotide fragment which may consist of 5 to 100 nucleotides, preferably 15 to 30 nucleotides, and having hybridization specificity with a target nucleotide sequence under conditions determined for the initiation of enzymatic polymerization, for example in a reaction for the enzymatic amplification of the target nucleotide sequence. Generally use is made of “primer pairs” consisting of two primers. When it is desired to amplify several different biomarkers (e.g. from different genes), several different pairs of primers are preferably used, each preferentially having the ability to hybridize specifically with a different biomarker.

“Probe” or “hybridization probe” means a nucleotide fragment typically consisting of 5 to 100 nucleotides, preferably 15 to 90 nucleotides, even more preferably 15 to 35 nucleotides, having hybridization specificity under conditions determined for forming a hybridization complex with a target nucleotide sequence. The probe also comprises a reporter (such as a fluorophore, an enzyme or any other detection system) which will enable the detection of the target nucleotide sequence. In the present invention, the target nucleotide sequence may be a nucleotide sequence contained in a messenger RNA (mRNA) or a nucleotide sequence contained in a complementary DNA (cDNA) obtained by reverse transcription of said mRNA. When it is desired to target several different biomarkers (e.g. from different genes), several different probes are preferably used, each preferentially having the ability to hybridize specifically with a different biomarker.

“Hybridization” means the process during which, under suitable conditions, two nucleotide fragments, for example a hybridization probe and a target nucleotide fragment, having sufficiently complementary sequences, are able to form a double strand with stable and specific hydrogen bonds. A nucleotide fragment which is “able to hybridize” with a polynucleotide is a fragment which can hybridize with said polynucleotide under hybridization conditions, which can be determined in each case, as is known. The hybridization conditions are determined by stringency, i.e. the strictness of the operating conditions. Hybridization is more specific when it is carried out at higher stringency levels. The stringency is defined particularly on the basis of the base composition of a probe/target duplex, and also by the degree of mismatch between two nucleic acids. The stringency can also be based on the reaction parameters, such as the concentration and type of the ionic species present in the hybridization solution, the nature and the concentration of denaturing agents, and/or the hybridization temperature. The stringency of the conditions under which a hybridization reaction must be carried out will chiefly depend on the hybridization probes used. All this information is well known and the suitable conditions can be determined by those skilled in the art. In general, depending on the length of the hybridization probes used, the temperature for the hybridization reaction is between approximately 20 and 70° C., in particular between 35 and 65° C. in a saline solution at a concentration of approximately 0.5 to 1 M. A step of detecting the hybridization reaction is subsequently carried out.

“Enzymatic amplification reaction” is intended to mean a process which generates multiple copies of a target nucleotide fragment by the action of at least one enzyme. Such amplification reactions are well known to those skilled in the art and mention may particularly be made of the following techniques: PCR (Polymerase Chain Reaction), LCR (Ligase Chain Reaction), RCR (Repair Chain Reaction), 3SR (Self Sustained Sequence Replication) with patent application WO-A-90/06995, NASBA (Nucleic Acid Sequence-Based Amplification), TMA (Transcription Mediated Amplification) with U.S. Pat. No. 5,399,491, and LAMP (Loop mediated isothermal amplification) with U.S. Pat. No. 6,410,278. When the enzymatic amplification reaction is PCR, reference will more particularly be made to RT-PCR (RT for reverse transcription) when the amplification step is preceded by a step of reverse transcription of messenger RNA (mRNA) to give complementary DNA (cDNA), and to qPCR or RT-qPCR when the PCR is quantitative.

Another subject of the invention is the use:

of means for amplifying (e.g. primers) and/or means for detecting (e.g. probes) the expression of CD177 and optionally also the expression of one or more other gene(s), as indicated previously, in all the embodiments (and particularly preferably one, two, three, four, five, six, seven, eight, nine, ten or eleven genes selected from the group consisting of: CD74, CIITA, IFNG, IL1R2, C3AR1, TAP2, HP, CX3R1, S100A9, CTLA4 and CD3D); or
of a kit comprising such amplification and/or detection means, and preferably all the amplification and/or detection means of said kit enable the detection and/or amplification of at most 100, 90, 80, 70, 60, 50, 40, 30, 29, 28, 27, 26, 25, 24, 23, 22, 21, 20, 19, 18, 17, 16, 15, 14, 13, 12, 11, 10, 9, 8, 7, 6, 5, 4, 3, 2 biomarkers in total, and optionally said kit also comprises means for amplifying and/or for detecting the expression of one or more housekeeping genes (preferably selected from the list consisting of: DECR1, HPRT1 and PPIB), in order to determine the risk of incidence of a healthcare-associated infection, preferably of a nosocomial infection, in a patient, preferably a patient within a healthcare facility, more preferably still within a hospital, more preferably still within the emergency department, the resuscitation unit, an intensive care unit or an ongoing care unit; particularly preferably, the patient is a patient in a septic state (more particularly in septic shock), a patient suffering from burns (more particularly severe burns), a patient suffering from an injury (more particularly a severe injury), or a patient undergoing surgery (more particularly major surgery).

The present invention is illustrated, non-limitingly, by the following examples.

### Example 1: Measurement of the Expression of Cd177 Makes it Possible to Predict the Risk of Incidence of a Healthcare-Associated Infection in a Patient

Materials and Methods A prospective, longitudinal, single-center observational clinical study was carried out at Hôpital Edouard Herriot (Lyon, France). The design of this clinical study was published in Rol et al (2017), BMJ Open 7(6): e015734. The clinical study was approved by the Agence Nationale de Sécurité du Médicament et des produits de santé (ANSM) [French Agency for the Safety of Drugs and Health Products] and the Comité de Protection des Personnes Sud-Est II [South-East II Independent Ethics Committee] in December 2015. Amendments to the protocol were made in July 2016 and January 2017. In brief, a total of 377 patients, in a septic state (n=35) or in septic shock (n=72), suffering from severe burns (n=24), from severe injury (n=137) or hospitalized in a resuscitation unit or an intensive care unit following major surgery (n=109), and 175 healthy volunteers, were included between December 2015 and March 2018.
Patients in a septic state/in septic shock: according to the first clinical protocol, only those patients in septic shock were included, on the basis of a suspected infected site, treatment with catecholamimes was started within the 48 h following admission to resuscitation, and treatment with catecholamines (noradrenalin)>0.25 μg/kg/min for at least 2 hours. Subsequently, the eligibility criteria were modified in August 2016, following the publication of a new definition of septic shock, Sepsis 3 (Singer et al (2016), JAMA 810-801:(8)315). Patients in septic shock were therefore included on the basis of a suspected infected site, treatment with catecholamimes was started within the 48 h following admission to resuscitation, along with vasopressive therapy required to maintain arterial pressure of ≥65 mm Hg and lactate concentration of >2 mmol/l (18 mg/dl), despite correction of hypovolemia. In 2017, the possibility of including patients in a septic state (according to the definition of Sepsis 3) was added, namely a suspected infected site and an increase in the SOFA score 2 points compared to the baseline SOFA in the 48 h following admission to resuscitation. For this population, day 1 corresponds to the day of diagnosis of sepsis or septic shock.
Severe injury: in the first protocol, only those patients with a sever injury were included (Injury Severity Score (ISS)≥25). In August 2016, the possibility of also including less sever injuries (16<ISS<24) was added. For this population, day 1 corresponds to the day of admission to the resuscitation unit or to the intensive care unit (˜day of the injury).
Major surgery: in the first protocol, only esogastrectomy, Bricker-type bladder resection, cephalic duodenopancreatectomy and abdominal aorta surgery by laparotomy were considered. Other types of surgery with a high risk of complications were added in January 2017: pancreatectomy (total or caudal), neuroendocrine tumors, hepatectomy (on the right-hand side), extended colectomy (laparotomy), abdoperineal resection, nephrectomy (laparotomy, PKD), ilio-femoral bypass (Scarpa). For this population, day 1 corresponds to the day of surgery.
Severe burns: the patients were selected on the basis of a total burn surface area of greater than 30%. For this population, day 1 corresponds to the day of admission to the resuscitation unit or to the intensive care unit (˜day of the burn).

The exclusion criteria related essentially to factors which could have impacted the immune status and could have biased the results (for example: severe neutropenia, corticosteroid treatments, an onco-haematological disease, etc.). Each event leading to a suspected healthcare-associated infection, occurring within the hospital before D30, was reviewed independently by three physicians who were not involved in patient recruitment. Twenty-six percent of the patients developed at least one healthcare-associated infection before D30, or before leaving hospital.

Blood samples were collected in PAXgene® tubes (ref. 762165, PreAnalytiX GmbH Hombrechtikon Switzerland), once for healthy volunteers and several times for a sub-cohort of 242 patients (i.e. 82 patients in a septic state/in septic shock, 83 patients suffering from severe injuries, 61 patients hospitalized in a resuscitation unit or an intensive care unit after major surgery, and 16 patients suffering from severe burns), i.e. 3-4 times in the first week (on days 1 or 2: D1/2, on days 3 or 4: D3/4, and on days 5, 6 or 7: D5/7), then 3 times at later times (around D14, D28 and D60).

The expression level of CD177 was measured in these samples by RT-qPCR. A volume of 100 μl of blood collected in the PAXgene® was directly injected into a FilmArray® pouch optimized for detecting a panel of genes involved in the host response, including CD177, by nested PCR. The steps of extraction of the nucleic acids, reverse transcription and qPCR were carried out sequentially and automatically by the Filmarray® instrument, without external intervention. The cycle thresholds (Ct) determined by the instrument were normalized in relation to the expression of 3 reference genes (DECR1, HPRT1 and PPIB).

Regarding data analysis, associations between the expression of CD177, measured at different times during the first week, and the incidence of a healthcare-associated infection before D30 from inclusion in the study were evaluated. The results were calculated in the form of Hazard Ratios expressed as the inter-quartile distance with the associated 95% confidence interval (HR IQR). Next, univariate logistic regression was implemented in order to predict the risk of incidence of a healthcare-associated infection before D15. The power of the values predicted by logistic regression to distinguish between healthcare-associated infection and lack of healthcare-associated infection was quantified by the area under the curve (AUC) of the ROC curve (Receiver Operating Characteristic), and 95% confidence intervals were estimated.

Next, the association between the expression of CD177 and the incidence of a healthcare-associated infection was evaluated for different time intervals of incidence of the infection (i.e. periods between taking the sample and the 1st incidence of an infection). The different periods considered were: a healthcare-associated infection in the 4 days and in the 7 days following the sample being taken, regardless of when the sample was taken. For each patient who developed a healthcare-associated infection, the sample considered corresponds to the sample taken closest to the incidence of the first episode of healthcare-associated infection.

For patients who did not develop a healthcare-associated infection (i.e. control patients), a matching method was used to select, for each case, a control patient whose sample was taken on the same day and with close SOFA and Charlson scores. Finally, a single control was selected for each unique case. Univariate logistic regressions were implemented. The power of the values predicted by logistic regression to distinguish between healthcare-associated infection and lack of healthcare-associated infection was quantified by the area under the curve (AUC) of the ROC curve, and 95% confidence intervals were estimated.

Results

An increase in the expression of CD177 at the mRNA level, measured on D3/4 or on D5/7 from inclusion in the cohort, was associated with a greater risk of incidence of a healthcare-associated infection before D30 in the overall patient population (univariate analysis on D3/4: HR IQR=1.64 [1.05-2.55], p=0.0291; on D5/7: HR IQR=2.30 [1.32-4.02], p=0.0034). This association was always significant for the measurement time on D5/7 after adjustment with the SOFA and Charlson scores (multivariate analysis: HR IQR=2.14 [1.20-3.84], p=0.0104).

Moreover, the prediction models showed that the expression of CD177 at the mRNA level, measured on D3/4 or D5.7 from inclusion in the cohort, made it possible to predict the incidence of a healthcare-associated infection before D15 from inclusion in the cohort (table 4).

TABLE 4

| Day on which | | | |
|---|---|---|---|
| sample was taken | AUC | AUC.2.5 | AUC.97.5 |
| D3/4 | 0.599 | 0.49 | 0.708 |
| D5/7 | 0.674 | 0.549 | 0.798 |

Table 4 Performance (AUC and 95% confidence intervals, with AUC2.5 and AUC 97.5 limits) of the measurement of the expression of CD177, measured on D3/4 or D5/7 from inclusion in the cohort, in predicting the incidence of a healthcare-associated infection before D15 from inclusion in the cohort in patients in a septic state/in septic shock, suffering from severe injuries, or hospitalized following major surgery.

The prediction models also showed that the expression of CD177 at the mRNA level made it possible to predict the incidence of a healthcare-associated infection in the 4 days or 7 days following the sample being taken (table 5).

TABLE 5

| Maximum time interval between the | | | |
|---|---|---|---|
| sample being taken and the potential | | | |
| incidence of first healthcare- | | | |
| associated infection | AUC | AUC.2.5 | AUC.97.5 |
| 4 days | 0.684 | 0.493 | 0.875 |
| 7 days | 0.726 | 0.56 | 0.892 |

Table 5 Performance (AUC and 95% confidence intervals, with AUC2.5 and AUC 97.5 limits) of the measurement of the expression of CD177 in predicting the incidence of a healthcare-associated infection in the 4 days or in the 7 days following the sample being taken.

Thus, the results obtained show that the measurement of the expression of CD177 alone makes it possible to predict the incidence of healthcare-associated infection(s) in the 15 days starting from the immuno-inflammatory attack, in the 4 days following the sample being taken or in the 7 days following the sample being taken.

### Example 2: The Measurement of the Expression of One or More Other Gene(s) in Combination with the Measurement of the Expression of CD177 Makes it Possible to Improve the Performance in Predicting the Risk of Incidence of a Healthcare-Associated Infection

In this example, the expression level of CD177 and also that of other genes was measured by RT-qPCR. Multivariate logistic regressions (combination of the measurement of the expression of CD177 and of one or more other gene(s)) were then carried out. The measurement of the expression of one or more of these other genes, in addition to the measurement of the expression of CD177, makes it possible to improve the performance (compared to the measurement of the expression of CD177 alone) in predicting the risk of incidence of a healthcare-associated infection, whether before D15 starting from inclusion in the cohort (table 6) or in the 4 days or in the 7 days following the sample being taken (table 7).

TABLE 6

| Day on | | | | |
|---|---|---|---|---|
| which | | | | |
| sample | | | | |
| was | | | | |
| taken | Biomarkers | AUC | AUC · 2.5 | AUC · 97.5 |
| D 3/4 | CD177 + GNLY | 0.601 | 0.493 | 0.709 |
| D 3/4 | CD177 + LILRB2 | 0.601 | 0.493 | 0.709 |
| D 3/4 | CD177 + CIITA | 0.602 | 0.494 | 0.71 |
| D 3/4 | CD177 + ZAP70 | 0.602 | 0.495 | 0.71 |
| D 3/4 | CD177 + CX3CR1 | 0.604 | 0.491 | 0.716 |
| D 3/4 | CD177 + CD274 | 0.604 | 0.495 | 0.713 |
| D 3/4 | CD177 + TAP2 | 0.604 | 0.496 | 0.712 |
| D 3/4 | CD177 + CTLA4 | 0.605 | 0.496 | 0.714 |
| D 3/4 | CD177 + FLT1 | 0.605 | 0.499 | 0.711 |
| D 3/4 | CD177 + IL6 | 0.605 | 0.495 | 0.714 |
| D 3/4 | CD177 + CXCL10 | 0.606 | 0.496 | 0.716 |
| D 3/4 | CD177 + IL1B | 0.61 | 0.506 | 0.715 |
| D 3/4 | CD177 + IL1RN | 0.612 | 0.503 | 0.721 |
| D 3/4 | CD177 + MDC1 | 0.618 | 0.515 | 0.721 |
| D 3/4 | CD177 + ALOX5 | 0.619 | 0.513 | 0.726 |
| D 3/4 | CD177 + CD74 | 0.621 | 0.511 | 0.73 |
| D 3/4 | CD177 + IL7R | 0.622 | 0.506 | 0.738 |
| D 3/4 | CD177 + GSN | 0.627 | 0.522 | 0.733 |
| D 3/4 | CD177 + ARL14EP | 0.633 | 0.509 | 0.756 |
| D 3/4 | CD177 + BPGM | 0.633 | 0.528 | 0.738 |
| D 3/4 | CD177 + HAVCR2 | 0.64 | 0.523 | 0.757 |
| D 3/4 | CD177 + S100A9 | 0.642 | 0.535 | 0.75 |
| D 3/4 | CD177 + IL2 | 0.648 | 0.544 | 0.752 |
| D 3/4 | CD177 + CCNB1IP1 | 0.65 | 0.528 | 0.772 |
| D 3/4 | CD177 + TDRD9 | 0.652 | 0.548 | 0.757 |
| D 3/4 | CD177 + IL10 | 0.671 | 0.562 | 0.781 |
| D 3/4 | CD177 + IL18 | 0.706 | 0.604 | 0.808 |
| D 3/4 | CD177 + C3AR1 | 0.626 | 0.516 | 0.735 |
| D 3/4 | CD177 + C3AR1 + CD3D | 0.627 | 0.517 | 0.737 |
| D 3/4 | CD177 + C3AR1 + CD3D + CTLA4 | 0.636 | 0.525 | 0.747 |
| D 3/4 | CD177 + C3AR1 + CD74 | 0.666 | 0.552 | 0.779 |
| D 3/4 | CD177 + C3AR1 + CD74 + CD3D | 0.669 | 0.551 | 0.786 |
| D 3/4 | CD177 + C3AR1 + CD74 + CD3D + CTLA4 | 0.664 | 0.548 | 0.781 |
| D 3/4 | CD177 + C3AR1 + CD74 + CIITA | 0.664 | 0.551 | 0.777 |
| D 3/4 | CD177 + C3AR1 + CD74 + CIITA + CD3D | 0.664 | 0.549 | 0.778 |
| D 3/4 | CD177 + C3AR1 + CD74 + CIITA + CD3D + CTLA4 | 0.66 | 0.547 | 0.773 |
| D 3/4 | CD177 + C3AR1 + CD74 + CIITA + CTLA4 | 0.656 | 0.546 | 0.767 |
| D 3/4 | CD177 + C3AR1 + CD74 + CIITA + TAP2 | 0.661 | 0.549 | 0.772 |
| D 3/4 | CD177 + C3AR1 + CD74 + CIITA + TAP2 + CD3D | 0.66 | 0.547 | 0.774 |
| D 3/4 | CD177 + C3AR1 + CD74 + CIITA + TAP2 + CD3D + CTLA4 | 0.666 | 0.553 | 0.779 |
| D 3/4 | CD177 + C3AR1 + CD74 + CIITA + TAP2 + CTLA4 | 0.66 | 0.549 | 0.771 |
| D 3/4 | CD177 + C3AR1 + CD74 + CTLA4 | 0.665 | 0.551 | 0.779 |
| D 3/4 | CD177 + C3AR1 + CD74 + CX3CR1 | 0.66 | 0.546 | 0.774 |
| D 3/4 | CD177 + C3AR1 + CD74 + CX3CR1 + CD3D | 0.664 | 0.548 | 0.78 |
| D 3/4 | CD177 + C3AR1 + CD74 + CX3CR1 + CD3D + CTLA4 | 0.662 | 0.546 | 0.778 |
| D 3/4 | CD177 + C3AR1 + CD74 + CX3CR1 + CIITA | 0.662 | 0.55 | 0.775 |
| D 3/4 | CD177 + C3AR1 + CD74 + CX3CR1 + CIITA + CD3D | 0.664 | 0.55 | 0.778 |
| D 3/4 | CD177 + C3AR1 + CD74 + CX3CR1 + CIITA + CD3D + CTLA4 | 0.663 | 0.551 | 0.776 |
| D 3/4 | CD177 + C3AR1 + CD74 + CX3CR1 + CIITA + CTLA4 | 0.658 | 0.547 | 0.769 |
| D 3/4 | CD177 + C3AR1 + CD74 + CX3CR1 + CIITA + TAP2 | 0.662 | 0.549 | 0.774 |
| D 3/4 | CD177 + C3AR1 + CD74 + CX3CR1 + CIITA + TAP2 + CD3D | 0.659 | 0.544 | 0.774 |
| D 3/4 | CD177 + C3AR1 + CD74 + CX3CR1 + CIITA + TAP2 + CD3D + CTLA4 | 0.665 | 0.552 | 0.778 |
| D 3/4 | CD177 + C3AR1 + CD74 + CX3CR1 + CIITA + TAP2 + CTLA4 | 0.657 | 0.545 | 0.768 |
| D 3/4 | CD177 + C3AR1 + CD74 + CX3CR1 + CTLA4 | 0.66 | 0.546 | 0.774 |
| D 3/4 | CD177 + C3AR1 + CD74 + CX3CR1 + TAP2 | 0.661 | 0.547 | 0.775 |
| D 3/4 | CD177 + C3AR1 + CD74 + CX3CR1 + TAP2 + CD3D | 0.659 | 0.544 | 0.774 |
| D 3/4 | CD177 + C3AR1 + CD74 + CX3CR1 + TAP2 + CD3D + CTLA4 | 0.663 | 0.548 | 0.779 |
| D 3/4 | CD177 + C3AR1 + CD74 + CX3CR1 + TAP2 + CTLA4 | 0.661 | 0.547 | 0.774 |
| D 3/4 | CD177 + C3AR1 + CD74 + IFNG | 0.664 | 0.55 | 0.778 |
| D 3/4 | CD177 + C3AR1 + CD74 + IFNG + CD3D | 0.672 | 0.557 | 0.788 |
| D 3/4 | CD177 + C3AR1 + CD74 + IFNG + CD3D + CTLA4 | 0.664 | 0.548 | 0.78 |
| D 3/4 | CD177 + C3AR1 + CD74 + IFNG + CIITA | 0.654 | 0.543 | 0.765 |
| D 3/4 | CD177 + C3AR1 + CD74 + IFNG + CIITA + CD3D | 0.66 | 0.548 | 0.772 |
| D 3/4 | CD177 + C3AR1 + CD74 + IFNG + CIITA + CD3D + CTLA4 | 0.664 | 0.552 | 0.776 |
| D 3/4 | CD177 + C3AR1 + CD74 + IFNG + CIITA + CTLA4 | 0.655 | 0.544 | 0.767 |
| D 3/4 | CD177 + C3AR1 + CD74 + IFNG + CIITA + TAP2 | 0.656 | 0.545 | 0.767 |
| D 3/4 | CD177 + C3AR1 + CD74 + IFNG + CIITA + TAP2 + CD3D | 0.661 | 0.551 | 0.772 |
| D 3/4 | CD177 + C3AR1 + CD74 + IFNG + CIITA + TAP2 + CD3D + CTLA4 | 0.664 | 0.553 | 0.774 |
| D 3/4 | CD177 + C3AR1 + CD74 + IFNG + CIITA + TAP2 + CTLA4 | 0.653 | 0.543 | 0.764 |
| D 3/4 | CD177 + C3AR1 + CD74 + IFNG + CTLA4 | 0.663 | 0.549 | 0.776 |
| D 3/4 | CD177 + C3AR1 + CD74 + IFNG + CX3CR1 | 0.665 | 0.551 | 0.779 |
| D 3/4 | CD177 + C3AR1 + CD74 + IFNG + CX3CR1 + CD3D | 0.664 | 0.55 | 0.779 |
| D 3/4 | CD177 + C3AR1 + CD74 + IFNG + CX3CR1 + CD3D + CTLA4 | 0.664 | 0.549 | 0.779 |
| D 3/4 | CD177 + C3AR1 + CD74 + IFNG + CX3CR1 + CIITA | 0.657 | 0.547 | 0.768 |
| D 3/4 | CD177 + C3AR1 + CD74 + IFNG + CX3CR1 + CIITA + CD3D | 0.659 | 0.546 | 0.771 |
| D 3/4 | CD177 + C3AR1 + CD74 + IFNG + CX3CR1 + CIITA + CD3D + CTLA4 | 0.663 | 0.551 | 0.775 |
| D 3/4 | CD177 + C3AR1 + CD74 + IFNG + CX3CR1 + CIITA + CTLA4 | 0.654 | 0.544 | 0.764 |
| D 3/4 | CD177 + C3AR1 + CD74 + IFNG + CX3CR1 + CIITA + TAP2 | 0.655 | 0.546 | 0.765 |
| D 3/4 | CD177 + C3AR1 + CD74 + IFNG + CX3CR1 + CIITA + TAP2 + CD3D | 0.657 | 0.546 | 0.768 |
| D 3/4 | CD177 + C3AR1 + CD74 + IFNG + CX3CR1 + CIITA + TAP2 + CD3D + CTLA4 | 0.66 | 0.549 | 0.771 |
| D 3/4 | CD177 + C3AR1 + CD74 + IFNG + CX3CR1 + CIITA + TAP2 + CTLA4 | 0.654 | 0.544 | 0.763 |
| D 3/4 | CD177 + C3AR1 + CD74 + IFNG + CX3CR1 + CTLA4 | 0.665 | 0.551 | 0.779 |
| D 3/4 | CD177 + C3AR1 + CD74 + IFNG + CX3CR1 + TAP2 | 0.665 | 0.551 | 0.778 |
| D 3/4 | CD177 + C3AR1 + CD74 + IFNG + CX3CR1 + TAP2 + CD3D | 0.664 | 0.549 | 0.778 |
| D 3/4 | CD177 + C3AR1 + CD74 + IFNG + CX3CR1 + TAP2 + CD3D + CTLA4 | 0.664 | 0.548 | 0.779 |
| D 3/4 | CD177 + C3AR1 + CD74 + IFNG + CX3CR1 + TAP2 + CTLA4 | 0.665 | 0.551 | 0.778 |
| D 3/4 | CD177 + C3AR1 + CD74 + IFNG + S100A9 | 0.664 | 0.548 | 0.78 |
| D 3/4 | CD177 + C3AR1 + CD74 + IFNG + S100A9 + CD3D | 0.671 | 0.555 | 0.787 |
| D 3/4 | CD177 + C3AR1 + CD74 + IFNG + S100A9 + CD3D + CTLA4 | 0.663 | 0.548 | 0.779 |
| D 3/4 | CD177 + C3AR1 + CD74 + IFNG + S100A9 + CIITA | 0.662 | 0.543 | 0.781 |
| D 3/4 | CD177 + C3AR1 + CD74 + IFNG + S100A9 + CIITA + CD3D | 0.662 | 0.545 | 0.78 |
| D 3/4 | CD177 + C3AR1 + CD74 + IFNG + S100A9 + CIITA + CD3D + CTLA4 | 0.66 | 0.544 | 0.776 |
| D 3/4 | CD177 + C3AR1 + CD74 + IFNG + S100A9 + CIITA + CTLA4 | 0.659 | 0.541 | 0.776 |
| D 3/4 | CD177 + C3AR1 + CD74 + IFNG + S100A9 + CIITA + TAP2 | 0.664 | 0.547 | 0.782 |
| D 3/4 | CD177 + C3AR1 + CD74 + IFNG + S100A9 + CIITA + TAP2 + CD3D | 0.662 | 0.547 | 0.777 |
| D 3/4 | CD177 + C3AR1 + CD74 + IFNG + S100A9 + CIITA + TAP2 + CD3D + CTLA4 | 0.664 | 0.551 | 0.778 |
| D 3/4 | CD177 + C3AR1 + CD74 + IFNG + S100A9 + CIITA + TAP2 + CTLA4 | 0.66 | 0.544 | 0.777 |
| D 3/4 | CD177 + C3AR1 + CD74 + IFNG + S100A9 + CTLA4 | 0.662 | 0.547 | 0.778 |
| D 3/4 | CD177 + C3AR1 + CD74 + IFNG + S100A9 + CX3CR1 | 0.664 | 0.55 | 0.778 |
| D 3/4 | CD177 + C3AR1 + CD74 + IFNG + S100A9 + CX3CR1 + CD3D | 0.665 | 0.551 | 0.78 |
| D 3/4 | CD177 + C3AR1 + CD74 + IFNG + S100A9 + CX3CR1 + CD3D + CTLA4 | 0.663 | 0.548 | 0.777 |
| D 3/4 | CD177 + C3AR1 + CD74 + IFNG + S100A9 + CX3CR1 + CIITA | 0.662 | 0.546 | 0.779 |
| D 3/4 | CD177 + C3AR1 + CD74 + IFNG + S100A9 + CX3CR1 + CIITA + CD3D | 0.662 | 0.545 | 0.779 |
| D 3/4 | CD177 + C3AR1 + CD74 + IFNG + S100A9 + CX3CR1 + CIITA + CD3D + CTLA4 | 0.658 | 0.542 | 0.774 |
| D 3/4 | CD177 + C3AR1 + CD74 + IFNG + S100A9 + CX3CR1 + CIITA + CTLA4 | 0.664 | 0.548 | 0.779 |
| D 3/4 | CD177 + C3AR1 + CD74 + IFNG + S100A9 + CX3CR1 + CIITA + TAP2 | 0.659 | 0.544 | 0.774 |
| D 3/4 | CD177 + C3AR1 + CD74 + IFNG + S100A9 + CX3CR1 + CIITA + TAP2 + CD3D | 0.661 | 0.547 | 0.775 |
| D 3/4 | CD177 + C3AR1 + CD74 + IFNG + S100A9 + CX3CR1 + CIITA + TAP2 + CD3D + CTLA4 | 0.664 | 0.549 | 0.778 |
| D 3/4 | CD177 + C3AR1 + CD74 + IFNG + S100A9 + CX3CR1 + CIITA + TAP2 + CTLA4 | 0.662 | 0.548 | 0.777 |
| D 3/4 | CD177 + C3AR1 + CD74 + IFNG + S100A9 + CX3CR1 + CTLA4 | 0.665 | 0.551 | 0.779 |
| D 3/4 | CD177 + C3AR1 + CD74 + IFNG + S100A9 + CX3CR1 + TAP2 | 0.664 | 0.55 | 0.778 |
| D 3/4 | CD177 + C3AR1 + CD74 + IFNG + S100A9 + CX3CR1 + TAP2 + CD3D | 0.663 | 0.548 | 0.777 |
| D 3/4 | CD177 + C3AR1 + CD74 + IFNG + S100A9 + CX3CR1 + TAP2 + CD3D + CTLA4 | 0.661 | 0.546 | 0.776 |
| D 3/4 | CD177 + C3AR1 + CD74 + IFNG + S100A9 + CX3CR1 + TAP2 + CTLA4 | 0.664 | 0.55 | 0.778 |
| D 3/4 | CD177 + C3AR1 + CD74 + IFNG + S100A9 + TAP2 | 0.662 | 0.546 | 0.779 |
| D 3/4 | CD177 + C3AR1 + CD74 + IFNG + S100A9 + TAP2 + CD3D | 0.669 | 0.553 | 0.784 |
| D 3/4 | CD177 + C3AR1 + CD74 + IFNG + S100A9 + TAP2 + CD3D + CTLA4 | 0.666 | 0.552 | 0.78 |
| D 3/4 | CD177 + C3AR1 + CD74 + IFNG + S100A9 + TAP2 + CTLA4 | 0.661 | 0.545 | 0.777 |
| D 3/4 | CD177 + C3AR1 + CD74 + IFNG + TAP2 | 0.663 | 0.549 | 0.777 |
| D 3/4 | CD177 + C3AR1 + CD74 + IFNG + TAP2 + CD3D | 0.668 | 0.553 | 0.784 |
| D 3/4 | CD177 + C3AR1 + CD74 + IFNG + TAP2 + CD3D + CTLA4 | 0.665 | 0.55 | 0.78 |
| D 3/4 | CD177 + C3AR1 + CD74 + IFNG + TAP2 + CTLA4 | 0.661 | 0.548 | 0.774 |
| D 3/4 | CD177 + C3AR1 + CD74 + S100A9 | 0.663 | 0.547 | 0.78 |
| D 3/4 | CD177 + C3AR1 + CD74 + S100A9 + CD3D | 0.667 | 0.55 | 0.785 |
| D 3/4 | CD177 + C3AR1 + CD74 + S100A9 + CD3D + CTLA4 | 0.664 | 0.548 | 0.78 |
| D 3/4 | CD177 + C3AR1 + CD74 + S100A9 + CIITA | 0.664 | 0.544 | 0.783 |
| D 3/4 | CD177 + C3AR1 + CD74 + S100A9 + CIITA + CD3D | 0.662 | 0.543 | 0.782 |
| D 3/4 | CD177 + C3AR1 + CD74 + S100A9 + CIITA + CD3D + CTLA4 | 0.66 | 0.544 | 0.777 |
| D 3/4 | CD177 + C3AR1 + CD74 + S100A9 + CIITA + CTLA4 | 0.66 | 0.543 | 0.776 |
| D 3/4 | CD177 + C3AR1 + CD74 + S100A9 + CIITA + TAP2 | 0.663 | 0.546 | 0.781 |
| D 3/4 | CD177 + C3AR1 + CD74 + S100A9 + CIITA + TAP2 + CD3D | 0.667 | 0.55 | 0.784 |
| D 3/4 | CD177 + C3AR1 + CD74 + S100A9 + CIITA + TAP2 + CD3D + CTLA4 | 0.666 | 0.55 | 0.782 |
| D 3/4 | CD177 + C3AR1 + CD74 + S100A9 + CIITA + TAP2 + CTLA4 | 0.659 | 0.542 | 0.775 |
| D 3/4 | CD177 + C3AR1 + CD74 + S100A9 + CTLA4 | 0.663 | 0.547 | 0.78 |
| D 3/4 | CD177 + C3AR1 + CD74 + S100A9 + CX3CR1 | 0.663 | 0.548 | 0.778 |
| D 3/4 | CD177 + C3AR1 + CD74 + S100A9 + CX3CR1 + CD3D | 0.662 | 0.546 | 0.779 |
| D 3/4 | CD177 + C3AR1 + CD74 + S100A9 + CX3CR1 + CD3D + CTLA4 | 0.663 | 0.547 | 0.779 |
| D 3/4 | CD177 + C3AR1 + CD74 + S100A9 + CX3CR1 + CIITA | 0.666 | 0.548 | 0.785 |
| D 3/4 | CD177 + C3AR1 + CD74 + S100A9 + CX3CR1 + CIITA + CD3D | 0.666 | 0.548 | 0.785 |
| D 3/4 | CD177 + C3AR1 + CD74 + S100A9 + CX3CR1 + CIITA + CD3D + CTLA4 | 0.659 | 0.543 | 0.776 |
| D 3/4 | CD177 + C3AR1 + CD74 + S100A9 + CX3CR1 + CIITA + CTLA4 | 0.661 | 0.545 | 0.778 |
| D 3/4 | CD177 + C3AR1 + CD74 + S100A9 + CX3CR1 + CIITA + TAP2 | 0.662 | 0.546 | 0.779 |
| D 3/4 | CD177 + C3AR1 + CD74 + S100A9 + CX3CR1 + CIITA + TAP2 + CD3D | 0.664 | 0.546 | 0.781 |
| D 3/4 | CD177 + C3AR1 + CD74 + S100A9 + CX3CR1 + CIITA + TAP2 + CD3D + CTLA4 | 0.664 | 0.549 | 0.78 |
| D 3/4 | CD177 + C3AR1 + CD74 + S100A9 + CX3CR1 + CIITA + TAP2 + CTLA4 | 0.662 | 0.546 | 0.778 |
| D 3/4 | CD177 + C3AR1 + CD74 + S100A9 + CX3CR1 + CTLA4 | 0.662 | 0.547 | 0.776 |
| D 3/4 | CD177 + C3AR1 + CD74 + S100A9 + CX3CR1 + TAP2 | 0.663 | 0.548 | 0.778 |
| D 3/4 | CD177 + C3AR1 + CD74 + S100A9 + CX3CR1 + TAP2 + CD3D | 0.661 | 0.545 | 0.776 |
| D 3/4 | CD177 + C3AR1 + CD74 + S100A9 + CX3CR1 + TAP2 + CD3D + CTLA4 | 0.663 | 0.548 | 0.778 |
| D 3/4 | CD177 + C3AR1 + CD74 + S100A9 + CX3CR1 + TAP2 + CTLA4 | 0.662 | 0.548 | 0.777 |
| D 3/4 | CD177 + C3AR1 + CD74 + S100A9 + TAP2 | 0.663 | 0.546 | 0.779 |
| D 3/4 | CD177 + C3AR1 + CD74 + S100A9 + TAP2 + CD3D | 0.666 | 0.55 | 0.782 |
| D 3/4 | CD177 + C3AR1 + CD74 + S100A9 + TAP2 + CD3D + CTLA4 | 0.663 | 0.548 | 0.779 |
| D 3/4 | CD177 + C3AR1 + CD74 + S100A9 + TAP2 + CTLA4 | 0.662 | 0.546 | 0.778 |
| D 3/4 | CD177 + C3AR1 + CD74 + TAP2 | 0.663 | 0.549 | 0.777 |
| D 3/4 | CD177 + C3AR1 + CD74 + TAP2 + CD3D | 0.666 | 0.55 | 0.782 |
| D 3/4 | CD177 + C3AR1 + CD74 + TAP2 + CD3D + CTLA4 | 0.665 | 0.549 | 0.78 |
| D 3/4 | CD177 + C3AR1 + CD74 + TAP2 + CTLA4 | 0.662 | 0.548 | 0.776 |
| D 3/4 | CD177 + C3AR1 + CIITA | 0.626 | 0.517 | 0.735 |
| D 3/4 | CD177 + C3AR1 + CIITA + CD3D | 0.628 | 0.518 | 0.738 |
| D 3/4 | CD177 + C3AR1 + CIITA + CD3D + CTLA4 | 0.632 | 0.52 | 0.744 |
| D 3/4 | CD177 + C3AR1 + CIITA + CTLA4 | 0.616 | 0.506 | 0.726 |
| D 3/4 | CD177 + C3AR1 + CIITA + TAP2 | 0.646 | 0.545 | 0.747 |
| D 3/4 | CD177 + C3AR1 + CIITA + TAP2 + CD3D | 0.643 | 0.533 | 0.753 |
| D 3/4 | CD177 + C3AR1 + CIITA + TAP2 + CD3D + CTLA4 | 0.638 | 0.521 | 0.754 |
| D 3/4 | CD177 + C3AR1 + CIITA + TAP2 + CTLA4 | 0.63 | 0.525 | 0.736 |
| D 3/4 | CD177 + C3AR1 + CTLA4 | 0.627 | 0.519 | 0.735 |
| D 3/4 | CD177 + C3AR1 + CX3CR1 | 0.627 | 0.516 | 0.738 |
| D 3/4 | CD177 + C3AR1 + CX3CR1 + CD3D | 0.627 | 0.516 | 0.739 |
| D 3/4 | CD177 + C3AR1 + CX3CR1 + CD3D + CTLA4 | 0.636 | 0.525 | 0.748 |
| D 3/4 | CD177 + C3AR1 + CX3CR1 + CIITA | 0.626 | 0.515 | 0.736 |
| D 3/4 | CD177 + C3AR1 + CX3CR1 + CIITA + CD3D | 0.627 | 0.516 | 0.738 |
| D 3/4 | CD177 + C3AR1 + CX3CR1 + CIITA + CD3D + CTLA4 | 0.631 | 0.519 | 0.743 |
| D 3/4 | CD177 + C3AR1 + CX3CR1 + CIITA + CTLA4 | 0.62 | 0.511 | 0.73 |
| D 3/4 | CD177 + C3AR1 + CX3CR1 + CIITA + TAP2 | 0.637 | 0.53 | 0.745 |
| D 3/4 | CD177 + C3AR1 + CX3CR1 + CIITA + TAP2 + CD3D | 0.639 | 0.527 | 0.752 |
| D 3/4 | CD177 + C3AR1 + CX3CR1 + CIITA + TAP2 + CD3D + CTLA4 | 0.636 | 0.519 | 0.753 |
| D 3/4 | CD177 + C3AR1 + CX3CR1 + CIITA + TAP2 + CTLA4 | 0.634 | 0.527 | 0.741 |
| D 3/4 | CD177 + C3AR1 + CX3CR1 + CTLA4 | 0.626 | 0.518 | 0.735 |
| D 3/4 | CD177 + C3AR1 + CX3CR1 + TAP2 | 0.645 | 0.537 | 0.753 |
| D 3/4 | CD177 + C3AR1 + CX3CR1 + TAP2 + CD3D | 0.652 | 0.54 | 0.764 |
| D 3/4 | CD177 + C3AR1 + CX3CR1 + TAP2 + CD3D + CTLA4 | 0.651 | 0.536 | 0.766 |
| D 3/4 | CD177 + C3AR1 + CX3CR1 + TAP2 + CTLA4 | 0.641 | 0.533 | 0.749 |
| D 3/4 | CD177 + C3AR1 + IFNG | 0.626 | 0.517 | 0.736 |
| D 3/4 | CD177 + C3AR1 + IFNG + CD3D | 0.627 | 0.516 | 0.738 |
| D 3/4 | CD177 + C3AR1 + IFNG + CD3D + CTLA4 | 0.634 | 0.522 | 0.746 |
| D 3/4 | CD177 + C3AR1 + IFNG + CIITA | 0.625 | 0.515 | 0.734 |
| D 3/4 | CD177 + C3AR1 + IFNG + CIITA + CD3D | 0.627 | 0.516 | 0.738 |
| D 3/4 | CD177 + C3AR1 + IFNG + CIITA + CD3D + CTLA4 | 0.632 | 0.52 | 0.744 |
| D 3/4 | CD177 + C3AR1 + IFNG + CIITA + CTLA4 | 0.617 | 0.509 | 0.725 |
| D 3/4 | CD177 + C3AR1 + IFNG + CIITA + TAP2 | 0.642 | 0.54 | 0.745 |
| D 3/4 | CD177 + C3AR1 + IFNG + CIITA + TAP2 + CD3D | 0.644 | 0.534 | 0.754 |
| D 3/4 | CD177 + C3AR1 + IFNG + CIITA + TAP2 + CD3D + CTLA4 | 0.636 | 0.519 | 0.753 |
| D 3/4 | CD177 + C3AR1 + IFNG + CIITA + TAP2 + CTLA4 | 0.633 | 0.528 | 0.738 |
| D 3/4 | CD177 + C3AR1 + IFNG + CTLA4 | 0.622 | 0.515 | 0.73 |
| D 3/4 | CD177 + C3AR1 + IFNG + CX3CR1 | 0.625 | 0.513 | 0.737 |
| D 3/4 | CD177 + C3AR1 + IFNG + CX3CR1 + CD3D | 0.626 | 0.515 | 0.738 |
| D 3/4 | CD177 + C3AR1 + IFNG + CX3CR1 + CD3D + CTLA4 | 0.635 | 0.523 | 0.746 |
| D 3/4 | CD177 + C3AR1 + IFNG + CX3CR1 + CIITA | 0.625 | 0.513 | 0.737 |
| D 3/4 | CD177 + C3AR1 + IFNG + CX3CR1 + CIITA + CD3D | 0.625 | 0.512 | 0.737 |
| D 3/4 | CD177 + C3AR1 + IFNG + CX3CR1 + CIITA + CD3D + CTLA4 | 0.63 | 0.518 | 0.742 |
| D 3/4 | CD177 + C3AR1 + IFNG + CX3CR1 + CIITA + CTLA4 | 0.619 | 0.509 | 0.728 |
| D 3/4 | CD177 + C3AR1 + IFNG + CX3CR1 + CIITA + TAP2 | 0.636 | 0.529 | 0.744 |
| D 3/4 | CD177 + C3AR1 + IFNG + CX3CR1 + CIITA + TAP2 + CD3D | 0.64 | 0.527 | 0.753 |
| D 3/4 | CD177 + C3AR1 + IFNG + CX3CR1 + CIITA + TAP2 + CD3D + CTLA4 | 0.636 | 0.519 | 0.753 |
| D 3/4 | CD177 + C3AR1 + IFNG + CX3CR1 + CIITA + TAP2 + CTLA4 | 0.632 | 0.525 | 0.739 |
| D 3/4 | CD177 + C3AR1 + IFNG + CX3CR1 + CTLA4 | 0.623 | 0.514 | 0.731 |
| D 3/4 | CD177 + C3AR1 + IFNG + CX3CR1 + TAP2 | 0.647 | 0.539 | 0.755 |
| D 3/4 | CD177 + C3AR1 + IFNG + CX3CR1 + TAP2 + CD3D | 0.65 | 0.538 | 0.762 |
| D 3/4 | CD177 + C3AR1 + IFNG + CX3CR1 + TAP2 + CD3D + CTLA4 | 0.642 | 0.525 | 0.759 |
| D 3/4 | CD177 + C3AR1 + IFNG + CX3CR1 + TAP2 + CTLA4 | 0.636 | 0.528 | 0.743 |
| D 3/4 | CD177 + C3AR1 + IFNG + S100A9 | 0.65 | 0.534 | 0.765 |
| D 3/4 | CD177 + C3AR1 + IFNG + S100A9 + CD3D | 0.648 | 0.534 | 0.763 |
| D 3/4 | CD177 + C3AR1 + IFNG + S100A9 + CD3D + CTLA4 | 0.644 | 0.531 | 0.756 |
| D 3/4 | CD177 + C3AR1 + IFNG + S100A9 + CIITA | 0.643 | 0.527 | 0.759 |
| D 3/4 | CD177 + C3AR1 + IFNG + S100A9 + CIITA + CD3D | 0.641 | 0.525 | 0.757 |
| D 3/4 | CD177 + C3AR1 + IFNG + S100A9 + CIITA + CD3D + CTLA4 | 0.634 | 0.52 | 0.748 |
| D 3/4 | CD177 + C3AR1 + IFNG + S100A9 + CIITA + CTLA4 | 0.633 | 0.519 | 0.747 |
| D 3/4 | CD177 + C3AR1 + IFNG + S100A9 + CIITA + TAP2 | 0.649 | 0.538 | 0.761 |
| D 3/4 | CD177 + C3AR1 + IFNG + S100A9 + CIITA + TAP2 + CD3D | 0.647 | 0.535 | 0.76 |
| D 3/4 | CD177 + C3AR1 + IFNG + S100A9 + CIITA + TAP2 + CD3D + CTLA4 | 0.646 | 0.532 | 0.76 |
| D 3/4 | CD177 + C3AR1 + IFNG + S100A9 + CIITA + TAP2 + CTLA4 | 0.639 | 0.529 | 0.75 |
| D 3/4 | CD177 + C3AR1 + IFNG + S100A9 + CTLA4 | 0.642 | 0.531 | 0.753 |
| D 3/4 | CD177 + C3AR1 + IFNG + S100A9 + CX3CR1 | 0.651 | 0.535 | 0.767 |
| D 3/4 | CD177 + C3AR1 + IFNG + S100A9 + CX3CR1 + CD3D | 0.647 | 0.533 | 0.762 |
| D 3/4 | CD177 + C3AR1 + IFNG + S100A9 + CX3CR1 + CD3D + CTLA4 | 0.646 | 0.533 | 0.758 |
| D 3/4 | CD177 + C3AR1 + IFNG + S100A9 + CX3CR1 + CIITA | 0.647 | 0.53 | 0.763 |
| D 3/4 | CD177 + C3AR1 + IFNG + S100A9 + CX3CR1 + CIITA + CD3D | 0.642 | 0.526 | 0.758 |
| D 3/4 | CD177 + C3AR1 + IFNG + S100A9 + CX3CR1 + CIITA + CD3D + CTLA4 | 0.635 | 0.52 | 0.749 |
| D 3/4 | CD177 + C3AR1 + IFNG + S100A9 + CX3CR1 + CIITA + CTLA4 | 0.631 | 0.518 | 0.744 |
| D 3/4 | CD177 + C3AR1 + IFNG + S100A9 + CX3CR1 + CIITA + TAP2 | 0.644 | 0.53 | 0.757 |
| D 3/4 | CD177 + C3AR1 + IFNG + S100A9 + CX3CR1 + CIITA + TAP2 + CD3D | 0.644 | 0.53 | 0.759 |
| D 3/4 | CD177 + C3AR1 + IFNG + S100A9 + CX3CR1 + CIITA + TAP2 + CD3D + CTLA4 | 0.642 | 0.528 | 0.757 |
| D 3/4 | CD177 + C3AR1 + IFNG + S100A9 + CX3CR1 + CIITA + TAP2 + CTLA4 | 0.638 | 0.528 | 0.748 |
| D 3/4 | CD177 + C3AR1 + IFNG + S100A9 + CX3CR1 + CTLA4 | 0.641 | 0.53 | 0.752 |
| D 3/4 | CD177 + C3AR1 + IFNG + S100A9 + CX3CR1 + TAP2 | 0.65 | 0.536 | 0.764 |
| D 3/4 | CD177 + C3AR1 + IFNG + S100A9 + CX3CR1 + TAP2 + CD3D | 0.652 | 0.539 | 0.766 |
| D 3/4 | CD177 + C3AR1 + IFNG + S100A9 + CX3CR1 + TAP2 + CD3D + CTLA4 | 0.648 | 0.534 | 0.763 |
| D 3/4 | CD177 + C3AR1 + IFNG + S100A9 + CX3CR1 + TAP2 + CTLA4 | 0.65 | 0.537 | 0.762 |
| D 3/4 | CD177 + C3AR1 + IFNG + S100A9 + TAP2 | 0.65 | 0.537 | 0.763 |
| D 3/4 | CD177 + C3AR1 + IFNG + S100A9 + TAP2 + CD3D | 0.653 | 0.539 | 0.767 |
| D 3/4 | CD177 + C3AR1 + IFNG + S100A9 + TAP2 + CD3D + CTLA4 | 0.65 | 0.535 | 0.764 |
| D 3/4 | CD177 + C3AR1 + IFNG + S100A9 + TAP2 + CTLA4 | 0.647 | 0.536 | 0.758 |
| D 3/4 | CD177 + C3AR1 + IFNG + TAP2 | 0.645 | 0.541 | 0.749 |
| D 3/4 | CD177 + C3AR1 + IFNG + TAP2 + CD3D | 0.648 | 0.538 | 0.758 |
| D 3/4 | CD177 + C3AR1 + IFNG + TAP2 + CD3D + CTLA4 | 0.641 | 0.525 | 0.758 |
| D 3/4 | CD177 + C3AR1 + IFNG + TAP2 + CTLA4 | 0.637 | 0.53 | 0.743 |
| D 3/4 | CD177 + C3AR1 + S100A9 | 0.65 | 0.538 | 0.761 |
| D 3/4 | CD177 + C3AR1 + S100A9 + CD3D | 0.65 | 0.538 | 0.762 |
| D 3/4 | CD177 + C3AR1 + S100A9 + CD3D + CTLA4 | 0.648 | 0.538 | 0.758 |
| D 3/4 | CD177 + C3AR1 + S100A9 + CIITA | 0.642 | 0.527 | 0.757 |
| D 3/4 | CD177 + C3AR1 + S100A9 + CIITA + CD3D | 0.64 | 0.525 | 0.755 |
| D 3/4 | CD177 + C3AR1 + S100A9 + CIITA + CD3D + CTLA4 | 0.631 | 0.517 | 0.745 |
| D 3/4 | CD177 + C3AR1 + S100A9 + CIITA + CTLA4 | 0.628 | 0.515 | 0.741 |
| D 3/4 | CD177 + C3AR1 + S100A9 + CIITA + TAP2 | 0.644 | 0.534 | 0.754 |
| D 3/4 | CD177 + C3AR1 + S100A9 + CIITA + TAP2 + CD3D | 0.646 | 0.534 | 0.758 |
| D 3/4 | CD177 + C3AR1 + S100A9 + CIITA + TAP2 + CD3D + CTLA4 | 0.647 | 0.534 | 0.761 |
| D 3/4 | CD177 + C3AR1 + S100A9 + CIITA + TAP2 + CTLA4 | 0.637 | 0.527 | 0.747 |
| D 3/4 | CD177 + C3AR1 + S100A9 + CTLA4 | 0.644 | 0.536 | 0.752 |
| D 3/4 | CD177 + C3AR1 + S100A9 + CX3CR1 | 0.65 | 0.535 | 0.766 |
| D 3/4 | CD177 + C3AR1 + S100A9 + CX3CR1 + CD3D | 0.649 | 0.536 | 0.762 |
| D 3/4 | CD177 + C3AR1 + S100A9 + CX3CR1 + CD3D + CTLA4 | 0.644 | 0.533 | 0.754 |
| D 3/4 | CD177 + C3AR1 + S100A9 + CX3CR1 + CIITA | 0.647 | 0.53 | 0.764 |
| D 3/4 | CD177 + C3AR1 + S100A9 + CX3CR1 + CIITA + CD3D | 0.645 | 0.529 | 0.76 |
| D 3/4 | CD177 + C3AR1 + S100A9 + CX3CR1 + CIITA + CD3D + CTLA4 | 0.631 | 0.518 | 0.745 |
| D 3/4 | CD177 + C3AR1 + S100A9 + CX3CR1 + CIITA + CTLA4 | 0.629 | 0.515 | 0.742 |
| D 3/4 | CD177 + C3AR1 + S100A9 + CX3CR1 + CIITA + TAP2 | 0.644 | 0.53 | 0.758 |
| D 3/4 | CD177 + C3AR1 + S100A9 + CX3CR1 + CIITA + TAP2 + CD3D | 0.644 | 0.53 | 0.759 |
| D 3/4 | CD177 + C3AR1 + S100A9 + CX3CR1 + CIITA + TAP2 + CD3D + CTLA4 | 0.644 | 0.53 | 0.758 |
| D 3/4 | CD177 + C3AR1 + S100A9 + CX3CR1 + CIITA + TAP2 + CTLA4 | 0.638 | 0.528 | 0.748 |
| D 3/4 | CD177 + C3AR1 + S100A9 + CX3CR1 + CTLA4 | 0.644 | 0.533 | 0.754 |
| D 3/4 | CD177 + C3AR1 + S100A9 + CX3CR1 + TAP2 | 0.656 | 0.544 | 0.768 |
| D 3/4 | CD177 + C3AR1 + S100A9 + CX3CR1 + TAP2 + CD3D | 0.659 | 0.545 | 0.772 |
| D 3/4 | CD177 + C3AR1 + S100A9 + CX3CR1 + TAP2 + CD3D + CTLA4 | 0.65 | 0.537 | 0.763 |
| D 3/4 | CD177 + C3AR1 + S100A9 + CX3CR1 + TAP2 + CTLA4 | 0.656 | 0.545 | 0.767 |
| D 3/4 | CD177 + C3AR1 + S100A9 + TAP2 | 0.65 | 0.539 | 0.761 |
| D 3/4 | CD177 + C3AR1 + S100A9 + TAP2 + CD3D | 0.659 | 0.548 | 0.771 |
| D 3/4 | CD177 + C3AR1 + S100A9 + TAP2 + CD3D + CTLA4 | 0.652 | 0.539 | 0.766 |
| D 3/4 | CD177 + C3AR1 + S100A9 + TAP2 + CTLA4 | 0.647 | 0.539 | 0.755 |
| D 3/4 | CD177 + C3AR1 + TAP2 | 0.651 | 0.549 | 0.754 |
| D 3/4 | CD177 + C3AR1 + TAP2 + CD3D | 0.648 | 0.539 | 0.758 |
| D 3/4 | CD177 + C3AR1 + TAP2 + CD3D + CTLA4 | 0.649 | 0.534 | 0.764 |
| D 3/4 | CD177 + C3AR1 + TAP2 + CTLA4 | 0.64 | 0.535 | 0.746 |
| D 3/4 | CD177 + CD3D + CTLA4 | 0.61 | 0.498 | 0.721 |
| D 3/4 | CD177 + CD74 + CD3D | 0.636 | 0.52 | 0.752 |
| D 3/4 | CD177 + CD74 + CD3D + CTLA4 | 0.637 | 0.52 | 0.753 |
| D 3/4 | CD177 + CD74 + CIITA | 0.64 | 0.526 | 0.754 |
| D 3/4 | CD177 + CD74 + CIITA + CD3D | 0.64 | 0.525 | 0.754 |
| D 3/4 | CD177 + CD74 + CIITA + CD3D + CTLA4 | 0.637 | 0.522 | 0.752 |
| D 3/4 | CD177 + CD74 + CIITA + CTLA4 | 0.64 | 0.527 | 0.753 |
| D 3/4 | CD177 + CD74 + CIITA + TAP2 | 0.647 | 0.533 | 0.76 |
| D 3/4 | CD177 + CD74 + CIITA + TAP2 + CD3D | 0.641 | 0.523 | 0.759 |
| D 3/4 | CD177 + CD74 + CIITA + TAP2 + CD3D + CTLA4 | 0.64 | 0.522 | 0.758 |
| D 3/4 | CD177 + CD74 + CIITA + TAP2 + CTLA4 | 0.645 | 0.532 | 0.757 |
| D 3/4 | CD177 + CD74 + CTLA4 | 0.639 | 0.525 | 0.753 |
| D 3/4 | CD177 + CD74 + CX3CR1 | 0.631 | 0.516 | 0.746 |
| D 3/4 | CD177 + CD74 + CX3CR1 + CD3D | 0.636 | 0.52 | 0.752 |
| D 3/4 | CD177 + CD74 + CX3CR1 + CD3D + CTLA4 | 0.633 | 0.517 | 0.75 |
| D 3/4 | CD177 + CD74 + CX3CR1 + CIITA | 0.642 | 0.527 | 0.756 |
| D 3/4 | CD177 + CD74 + CX3CR1 + CIITA + CD3D | 0.64 | 0.525 | 0.755 |
| D 3/4 | CD177 + CD74 + CX3CR1 + CIITA + CD3D + CTLA4 | 0.638 | 0.524 | 0.753 |
| D 3/4 | CD177 + CD74 + CX3CR1 + CIITA + CTLA4 | 0.641 | 0.528 | 0.755 |
| D 3/4 | CD177 + CD74 + CX3CR1 + CIITA + TAP2 | 0.643 | 0.528 | 0.758 |
| D 3/4 | CD177 + CD74 + CX3CR1 + CIITA + TAP2 + CD3D | 0.642 | 0.524 | 0.76 |
| D 3/4 | CD177 + CD74 + CX3CR1 + CIITA + TAP2 + CD3D + CTLA4 | 0.642 | 0.524 | 0.76 |
| D 3/4 | CD177 + CD74 + CX3CR1 + CIITA + TAP2 + CTLA4 | 0.642 | 0.527 | 0.757 |
| D 3/4 | CD177 + CD74 + CX3CR1 + CTLA4 | 0.63 | 0.515 | 0.746 |
| D 3/4 | CD177 + CD74 + CX3CR1 + TAP2 | 0.637 | 0.522 | 0.751 |
| D 3/4 | CD177 + CD74 + CX3CR1 + TAP2 + CD3D | 0.636 | 0.519 | 0.753 |
| D 3/4 | CD177 + CD74 + CX3CR1 + TAP2 + CD3D + CTLA4 | 0.635 | 0.519 | 0.752 |
| D 3/4 | CD177 + CD74 + CX3CR1 + TAP2 + CTLA4 | 0.636 | 0.52 | 0.751 |
| D 3/4 | CD177 + CD74 + IFNG | 0.638 | 0.525 | 0.752 |
| D 3/4 | CD177 + CD74 + IFNG + CD3D | 0.636 | 0.521 | 0.752 |
| D 3/4 | CD177 + CD74 + IFNG + CD3D + CTLA4 | 0.635 | 0.519 | 0.751 |
| D 3/4 | CD177 + CD74 + IFNG + CIITA | 0.646 | 0.534 | 0.757 |
| D 3/4 | CD177 + CD74 + IFNG + CIITA + CD3D | 0.65 | 0.537 | 0.763 |
| D 3/4 | CD177 + CD74 + IFNG + CIITA + CD3D + CTLA4 | 0.649 | 0.537 | 0.761 |
| D 3/4 | CD177 + CD74 + IFNG + CIITA + CTLA4 | 0.643 | 0.532 | 0.755 |
| D 3/4 | CD177 + CD74 + IFNG + CIITA + TAP2 | 0.648 | 0.537 | 0.759 |
| D 3/4 | CD177 + CD74 + IFNG + CIITA + TAP2 + CD3D | 0.649 | 0.537 | 0.761 |
| D 3/4 | CD177 + CD74 + IFNG + CIITA + TAP2 + CD3D + CTLA4 | 0.648 | 0.536 | 0.759 |
| D 3/4 | CD177 + CD74 + IFNG + CIITA + TAP2 + CTLA4 | 0.647 | 0.537 | 0.757 |
| D 3/4 | CD177 + CD74 + IFNG + CTLA4 | 0.638 | 0.524 | 0.753 |
| D 3/4 | CD177 + CD74 + IFNG + CX3CR1 | 0.635 | 0.522 | 0.748 |
| D 3/4 | CD177 + CD74 + IFNG + CX3CR1 + CD3D | 0.636 | 0.522 | 0.751 |
| D 3/4 | CD177 + CD74 + IFNG + CX3CR1 + CD3D + CTLA4 | 0.636 | 0.521 | 0.751 |
| D 3/4 | CD177 + CD74 + IFNG + CX3CR1 + CIITA | 0.641 | 0.529 | 0.752 |
| D 3/4 | CD177 + CD74 + IFNG + CX3CR1 + CIITA + CD3D | 0.645 | 0.532 | 0.757 |
| D 3/4 | CD177 + CD74 + IFNG + CX3CR1 + CIITA + CD3D + CTLA4 | 0.647 | 0.535 | 0.758 |
| D 3/4 | CD177 + CD74 + IFNG + CX3CR1 + CIITA + CTLA4 | 0.64 | 0.529 | 0.751 |
| D 3/4 | CD177 + CD74 + IFNG + CX3CR1 + CIITA + TAP2 | 0.646 | 0.534 | 0.758 |
| D 3/4 | CD177 + CD74 + IFNG + CX3CR1 + CIITA + TAP2 + CD3D | 0.647 | 0.533 | 0.76 |
| D 3/4 | CD177 + CD74 + IFNG + CX3CR1 + CIITA + TAP2 + CD3D + CTLA4 | 0.646 | 0.532 | 0.76 |
| D 3/4 | CD177 + CD74 + IFNG + CX3CR1 + CIITA + TAP2 + CTLA4 | 0.648 | 0.536 | 0.76 |
| D 3/4 | CD177 + CD74 + IFNG + CX3CR1 + CTLA4 | 0.636 | 0.522 | 0.75 |
| D 3/4 | CD177 + CD74 + IFNG + CX3CR1 + TAP2 | 0.636 | 0.523 | 0.749 |
| D 3/4 | CD177 + CD74 + IFNG + CX3CR1 + TAP2 + CD3D | 0.642 | 0.528 | 0.756 |
| D 3/4 | CD177 + CD74 + IFNG + CX3CR1 + TAP2 + CD3D + CTLA4 | 0.639 | 0.525 | 0.753 |
| D 3/4 | CD177 + CD74 + IFNG + CX3CR1 + TAP2 + CTLA4 | 0.638 | 0.524 | 0.752 |
| D 3/4 | CD177 + CD74 + IFNG + S100A9 | 0.646 | 0.529 | 0.762 |
| D 3/4 | CD177 + CD74 + IFNG + S100A9 + CD3D | 0.645 | 0.528 | 0.761 |
| D 3/4 | CD177 + CD74 + IFNG + S100A9 + CD3D + CTLA4 | 0.643 | 0.527 | 0.759 |
| D 3/4 | CD177 + CD74 + IFNG + S100A9 + CIITA | 0.647 | 0.529 | 0.766 |
| D 3/4 | CD177 + CD74 + IFNG + S100A9 + CIITA + CD3D | 0.648 | 0.53 | 0.767 |
| D 3/4 | CD177 + CD74 + IFNG + S100A9 + CIITA + CD3D + CTLA4 | 0.648 | 0.529 | 0.766 |
| D 3/4 | CD177 + CD74 + IFNG + S100A9 + CIITA + CTLA4 | 0.649 | 0.53 | 0.768 |
| D 3/4 | CD177 + CD74 + IFNG + S100A9 + CIITA + TAP2 | 0.652 | 0.533 | 0.771 |
| D 3/4 | CD177 + CD74 + IFNG + S100A9 + CIITA + TAP2 + CD3D | 0.652 | 0.534 | 0.77 |
| D 3/4 | CD177 + CD74 + IFNG + S100A9 + CIITA + TAP2 + CD3D + CTLA4 | 0.653 | 0.536 | 0.771 |
| D 3/4 | CD177 + CD74 + IFNG + S100A9 + CIITA + TAP2 + CTLA4 | 0.65 | 0.531 | 0.769 |
| D 3/4 | CD177 + CD74 + IFNG + S100A9 + CTLA4 | 0.643 | 0.528 | 0.759 |
| D 3/4 | CD177 + CD74 + IFNG + S100A9 + CX3CR1 | 0.645 | 0.529 | 0.761 |
| D 3/4 | CD177 + CD74 + IFNG + S100A9 + CX3CR1 + CD3D | 0.645 | 0.53 | 0.76 |
| D 3/4 | CD177 + CD74 + IFNG + S100A9 + CX3CR1 + CD3D + CTLA4 | 0.644 | 0.529 | 0.759 |
| D 3/4 | CD177 + CD74 + IFNG + S100A9 + CX3CR1 + CIITA | 0.653 | 0.535 | 0.771 |
| D 3/4 | CD177 + CD74 + IFNG + S100A9 + CX3CR1 + CIITA + CD3D | 0.653 | 0.536 | 0.771 |
| D 3/4 | CD177 + CD74 + IFNG + S100A9 + CX3CR1 + CIITA + CD3D + CTLA4 | 0.653 | 0.535 | 0.771 |
| D 3/4 | CD177 + CD74 + IFNG + S100A9 + CX3CR1 + CIITA + CTLA4 | 0.654 | 0.536 | 0.772 |
| D 3/4 | CD177 + CD74 + IFNG + S100A9 + CX3CR1 + CIITA + TAP2 | 0.654 | 0.537 | 0.771 |
| D 3/4 | CD177 + CD74 + IFNG + S100A9 + CX3CR1 + CIITA + TAP2 + CD3D | 0.657 | 0.539 | 0.774 |
| D 3/4 | CD177 + CD74 + IFNG + S100A9 + CX3CR1 + CIITA + TAP2 + CD3D + CTLA4 | 0.654 | 0.538 | 0.771 |
| D 3/4 | CD177 + CD74 + IFNG + S100A9 + CX3CR1 + CIITA + TAP2 + CTLA4 | 0.653 | 0.536 | 0.77 |
| D 3/4 | CD177 + CD74 + IFNG + S100A9 + CX3CR1 + CTLA4 | 0.646 | 0.53 | 0.761 |
| D 3/4 | CD177 + CD74 + IFNG + S100A9 + CX3CR1 + TAP2 | 0.647 | 0.533 | 0.761 |
| D 3/4 | CD177 + CD74 + IFNG + S100A9 + CX3CR1 + TAP2 + CD3D | 0.642 | 0.526 | 0.758 |
| D 3/4 | CD177 + CD74 + IFNG + S100A9 + CX3CR1 + TAP2 + CD3D + CTLA4 | 0.643 | 0.527 | 0.76 |
| D 3/4 | CD177 + CD74 + IFNG + S100A9 + CX3CR1 + TAP2 + CTLA4 | 0.645 | 0.53 | 0.76 |
| D 3/4 | CD177 + CD74 + IFNG + S100A9 + TAP2 | 0.646 | 0.529 | 0.762 |
| D 3/4 | CD177 + CD74 + IFNG + S100A9 + TAP2 + CD3D | 0.643 | 0.529 | 0.758 |
| D 3/4 | CD177 + CD74 + IFNG + S100A9 + TAP2 + CD3D + CTLA4 | 0.645 | 0.53 | 0.759 |
| D 3/4 | CD177 + CD74 + IFNG + S100A9 + TAP2 + CTLA4 | 0.643 | 0.527 | 0.759 |
| D 3/4 | CD177 + CD74 + IFNG + TAP2 | 0.639 | 0.526 | 0.752 |
| D 3/4 | CD177 + CD74 + IFNG + TAP2 + CD3D | 0.635 | 0.521 | 0.75 |
| D 3/4 | CD177 + CD74 + IFNG + TAP2 + CD3D + CTLA4 | 0.636 | 0.522 | 0.75 |
| D 3/4 | CD177 + CD74 + IFNG + TAP2 + CTLA4 | 0.64 | 0.526 | 0.755 |
| D 3/4 | CD177 + CD74 + S100A9 | 0.645 | 0.529 | 0.76 |
| D 3/4 | CD177 + CD74 + S100A9 + CD3D | 0.645 | 0.527 | 0.762 |
| D 3/4 | CD177 + CD74 + S100A9 + CD3D + CTLA4 | 0.645 | 0.528 | 0.762 |
| D 3/4 | CD177 + CD74 + S100A9 + CIITA | 0.651 | 0.531 | 0.772 |
| D 3/4 | CD177 + CD74 + S100A9 + CIITA + CD3D | 0.651 | 0.53 | 0.772 |
| D 3/4 | CD177 + CD74 + S100A9 + CIITA + CD3D + CTLA4 | 0.647 | 0.526 | 0.767 |
| D 3/4 | CD177 + CD74 + S100A9 + CIITA + CTLA4 | 0.65 | 0.529 | 0.77 |
| D 3/4 | CD177 + CD74 + S100A9 + CIITA + TAP2 | 0.653 | 0.533 | 0.773 |
| D 3/4 | CD177 + CD74 + S100A9 + CIITA + TAP2 + CD3D | 0.653 | 0.532 | 0.774 |
| D 3/4 | CD177 + CD74 + S100A9 + CIITA + TAP2 + CD3D + CTLA4 | 0.648 | 0.528 | 0.769 |
| D 3/4 | CD177 + CD74 + S100A9 + CIITA + TAP2 + CTLA4 | 0.653 | 0.533 | 0.774 |
| D 3/4 | CD177 + CD74 + S100A9 + CTLA4 | 0.643 | 0.527 | 0.759 |
| D 3/4 | CD177 + CD74 + S100A9 + CX3CR1 | 0.646 | 0.529 | 0.762 |
| D 3/4 | CD177 + CD74 + S100A9 + CX3CR1 + CD3D | 0.644 | 0.527 | 0.761 |
| D 3/4 | CD177 + CD74 + S100A9 + CX3CR1 + CD3D + CTLA4 | 0.644 | 0.527 | 0.761 |
| D 3/4 | CD177 + CD74 + S100A9 + CX3CR1 + CIITA | 0.647 | 0.527 | 0.767 |
| D 3/4 | CD177 + CD74 + S100A9 + CX3CR1 + CIITA + CD3D | 0.651 | 0.53 | 0.771 |
| D 3/4 | CD177 + CD74 + S100A9 + CX3CR1 + CIITA + CD3D + CTLA4 | 0.648 | 0.528 | 0.769 |
| D 3/4 | CD177 + CD74 + S100A9 + CX3CR1 + CIITA + CTLA4 | 0.648 | 0.527 | 0.769 |
| D 3/4 | CD177 + CD74 + S100A9 + CX3CR1 + CIITA + TAP2 | 0.653 | 0.53 | 0.776 |
| D 3/4 | CD177 + CD74 + S100A9 + CX3CR1 + CIITA + TAP2 + CD3D | 0.653 | 0.529 | 0.776 |
| D 3/4 | CD177 + CD74 + S100A9 + CX3CR1 + CIITA + TAP2 + CD3D + CTLA4 | 0.648 | 0.526 | 0.771 |
| D 3/4 | CD177 + CD74 + S100A9 + CX3CR1 + CIITA + TAP2 + CTLA4 | 0.651 | 0.528 | 0.774 |
| D 3/4 | CD177 + CD74 + S100A9 + CX3CR1 + CTLA4 | 0.647 | 0.53 | 0.764 |
| D 3/4 | CD177 + CD74 + S100A9 + CX3CR1 + TAP2 | 0.644 | 0.529 | 0.76 |
| D 3/4 | CD177 + CD74 + S100A9 + CX3CR1 + TAP2 + CD3D | 0.638 | 0.521 | 0.756 |
| D 3/4 | CD177 + CD74 + S100A9 + CX3CR1 + TAP2 + CD3D + CTLA4 | 0.638 | 0.521 | 0.756 |
| D 3/4 | CD177 + CD74 + S100A9 + CX3CR1 + TAP2 + CTLA4 | 0.642 | 0.525 | 0.759 |
| D 3/4 | CD177 + CD74 + S100A9 + TAP2 | 0.643 | 0.528 | 0.759 |
| D 3/4 | CD177 + CD74 + S100A9 + TAP2 + CD3D | 0.642 | 0.527 | 0.758 |
| D 3/4 | CD177 + CD74 + S100A9 + TAP2 + CD3D + CTLA4 | 0.645 | 0.529 | 0.761 |
| D 3/4 | CD177 + CD74 + S100A9 + TAP2 + CTLA4 | 0.643 | 0.527 | 0.759 |
| D 3/4 | CD177 + CD74 + TAP2 | 0.639 | 0.526 | 0.752 |
| D 3/4 | CD177 + CD74 + TAP2 + CD3D | 0.636 | 0.52 | 0.751 |
| D 3/4 | CD177 + CD74 + TAP2 + CD3D + CTLA4 | 0.636 | 0.52 | 0.751 |
| D 3/4 | CD177 + CD74 + TAP2 + CTLA4 | 0.64 | 0.525 | 0.755 |
| D 3/4 | CD177 + CIITA + CD3D | 0.605 | 0.495 | 0.715 |
| D 3/4 | CD177 + CIITA + CD3D + CTLA4 | 0.611 | 0.5 | 0.722 |
| D 3/4 | CD177 + CIITA + CTLA4 | 0.605 | 0.496 | 0.714 |
| D 3/4 | CD177 + CIITA + TAP2 | 0.605 | 0.497 | 0.713 |
| D 3/4 | CD177 + CIITA + TAP2 + CD3D | 0.611 | 0.501 | 0.721 |
| D 3/4 | CD177 + CIITA + TAP2 + CD3D + CTLA4 | 0.612 | 0.498 | 0.725 |
| D 3/4 | CD177 + CIITA + TAP2 + CTLA4 | 0.605 | 0.496 | 0.714 |
| D 3/4 | CD177 + CX3CR1 + CD3D | 0.602 | 0.489 | 0.715 |
| D 3/4 | CD177 + CX3CR1 + CD3D + CTLA4 | 0.609 | 0.495 | 0.722 |
| D 3/4 | CD177 + CX3CR1 + CIITA | 0.616 | 0.505 | 0.726 |
| D 3/4 | CD177 + CX3CR1 + CIITA + CD3D | 0.616 | 0.504 | 0.727 |
| D 3/4 | CD177 + CX3CR1 + CIITA + CD3D + CTLA4 | 0.611 | 0.499 | 0.724 |
| D 3/4 | CD177 + CX3CR1 + CIITA + CTLA4 | 0.617 | 0.507 | 0.727 |
| D 3/4 | CD177 + CX3CR1 + CIITA + TAP2 | 0.62 | 0.508 | 0.732 |
| D 3/4 | CD177 + CX3CR1 + CIITA + TAP2 + CD3D | 0.618 | 0.503 | 0.732 |
| D 3/4 | CD177 + CX3CR1 + CIITA + TAP2 + CD3D + CTLA4 | 0.613 | 0.497 | 0.73 |
| D 3/4 | CD177 + CX3CR1 + CIITA + TAP2 + CTLA4 | 0.619 | 0.507 | 0.731 |
| D 3/4 | CD177 + CX3CR1 + CTLA4 | 0.612 | 0.501 | 0.723 |
| D 3/4 | CD177 + CX3CR1 + TAP2 | 0.614 | 0.501 | 0.727 |
| D 3/4 | CD177 + CX3CR1 + TAP2 + CD3D | 0.614 | 0.499 | 0.728 |
| D 3/4 | CD177 + CX3CR1 + TAP2 + CD3D + CTLA4 | 0.615 | 0.499 | 0.731 |
| D 3/4 | CD177 + CX3CR1 + TAP2 + CTLA4 | 0.615 | 0.503 | 0.727 |
| D 3/4 | CD177 + IFNG + CD3D | 0.602 | 0.491 | 0.713 |
| D 3/4 | CD177 + IFNG + CD3D + CTLA4 | 0.603 | 0.49 | 0.717 |
| D 3/4 | CD177 + IFNG + CIITA | 0.609 | 0.5 | 0.717 |
| D 3/4 | CD177 + IFNG + CIITA + CD3D | 0.607 | 0.496 | 0.718 |
| D 3/4 | CD177 + IFNG + CIITA + CD3D + CTLA4 | 0.61 | 0.497 | 0.722 |
| D 3/4 | CD177 + IFNG + CIITA + CTLA4 | 0.61 | 0.5 | 0.72 |
| D 3/4 | CD177 + IFNG + CIITA + TAP2 | 0.614 | 0.504 | 0.724 |
| D 3/4 | CD177 + IFNG + CIITA + TAP2 + CD3D | 0.608 | 0.496 | 0.721 |
| D 3/4 | CD177 + IFNG + CIITA + TAP2 + CD3D + CTLA4 | 0.609 | 0.493 | 0.725 |
| D 3/4 | CD177 + IFNG + CIITA + TAP2 + CTLA4 | 0.608 | 0.497 | 0.719 |
| D 3/4 | CD177 + IFNG + CTLA4 | 0.605 | 0.495 | 0.715 |
| D 3/4 | CD177 + IFNG + CX3CR1 | 0.602 | 0.489 | 0.716 |
| D 3/4 | CD177 + IFNG + CX3CR1 + CD3D | 0.601 | 0.488 | 0.714 |
| D 3/4 | CD177 + IFNG + CX3CR1 + CD3D + CTLA4 | 0.604 | 0.49 | 0.719 |
| D 3/4 | CD177 + IFNG + CX3CR1 + CIITA | 0.614 | 0.502 | 0.726 |
| D 3/4 | CD177 + IFNG + CX3CR1 + CIITA + CD3D | 0.615 | 0.503 | 0.726 |
| D 3/4 | CD177 + IFNG + CX3CR1 + CIITA + CD3D + CTLA4 | 0.613 | 0.5 | 0.726 |
| D 3/4 | CD177 + IFNG + CX3CR1 + CIITA + CTLA4 | 0.615 | 0.503 | 0.727 |
| D 3/4 | CD177 + IFNG + CX3CR1 + CIITA + TAP2 | 0.62 | 0.506 | 0.734 |
| D 3/4 | CD177 + IFNG + CX3CR1 + CIITA + TAP2 + CD3D | 0.617 | 0.502 | 0.732 |
| D 3/4 | CD177 + IFNG + CX3CR1 + CIITA + TAP2 + CD3D + CTLA4 | 0.611 | 0.493 | 0.729 |
| D 3/4 | CD177 + IFNG + CX3CR1 + CIITA + TAP2 + CTLA4 | 0.613 | 0.498 | 0.727 |
| D 3/4 | CD177 + IFNG + CX3CR1 + CTLA4 | 0.608 | 0.496 | 0.721 |
| D 3/4 | CD177 + IFNG + CX3CR1 + TAP2 | 0.611 | 0.497 | 0.726 |
| D 3/4 | CD177 + IFNG + CX3CR1 + TAP2 + CD3D | 0.611 | 0.494 | 0.727 |
| D 3/4 | CD177 + IFNG + CX3CR1 + TAP2 + CD3D + CTLA4 | 0.61 | 0.491 | 0.729 |
| D 3/4 | CD177 + IFNG + CX3CR1 + TAP2 + CTLA4 | 0.608 | 0.495 | 0.722 |
| D 3/4 | CD177 + IFNG + S100A9 | 0.645 | 0.528 | 0.762 |
| D 3/4 | CD177 + IFNG + S100A9 + CD3D | 0.645 | 0.528 | 0.762 |
| D 3/4 | CD177 + IFNG + S100A9 + CD3D + CTLA4 | 0.642 | 0.528 | 0.756 |
| D 3/4 | CD177 + IFNG + S100A9 + CIITA | 0.649 | 0.533 | 0.766 |
| D 3/4 | CD177 + IFNG + S100A9 + CIITA + CD3D | 0.646 | 0.529 | 0.763 |
| D 3/4 | CD177 + IFNG + S100A9 + CIITA + CD3D + CTLA4 | 0.639 | 0.524 | 0.753 |
| D 3/4 | CD177 + IFNG + S100A9 + CIITA + CTLA4 | 0.638 | 0.524 | 0.753 |
| D 3/4 | CD177 + IFNG + S100A9 + CIITA + TAP2 | 0.649 | 0.533 | 0.765 |
| D 3/4 | CD177 + IFNG + S100A9 + CIITA + TAP2 + CD3D | 0.644 | 0.526 | 0.761 |
| D 3/4 | CD177 + IFNG + S100A9 + CIITA + TAP2 + CD3D + CTLA4 | 0.637 | 0.523 | 0.752 |
| D 3/4 | CD177 + IFNG + S100A9 + CIITA + TAP2 + CTLA4 | 0.637 | 0.522 | 0.751 |
| D 3/4 | CD177 + IFNG + S100A9 + CTLA4 | 0.641 | 0.526 | 0.755 |
| D 3/4 | CD177 + IFNG + S100A9 + CX3CR1 | 0.645 | 0.527 | 0.763 |
| D 3/4 | CD177 + IFNG + S100A9 + CX3CR1 + CD3D | 0.641 | 0.524 | 0.758 |
| D 3/4 | CD177 + IFNG + S100A9 + CX3CR1 + CD3D + CTLA4 | 0.639 | 0.523 | 0.755 |
| D 3/4 | CD177 + IFNG + S100A9 + CX3CR1 + CIITA | 0.646 | 0.527 | 0.764 |
| D 3/4 | CD177 + IFNG + S100A9 + CX3CR1 + CIITA + CD3D | 0.643 | 0.525 | 0.76 |
| D 3/4 | CD177 + IFNG + S100A9 + CX3CR1 + CIITA + CD3D + CTLA4 | 0.637 | 0.521 | 0.753 |
| D 3/4 | CD177 + IFNG + S100A9 + CX3CR1 + CIITA + CTLA4 | 0.638 | 0.522 | 0.754 |
| D 3/4 | CD177 + IFNG + S100A9 + CX3CR1 + CIITA + TAP2 | 0.651 | 0.534 | 0.769 |
| D 3/4 | CD177 + IFNG + S100A9 + CX3CR1 + CIITA + TAP2 + CD3D | 0.645 | 0.526 | 0.763 |
| D 3/4 | CD177 + IFNG + S100A9 + CX3CR1 + CIITA + TAP2 + CD3D + CTLA4 | 0.64 | 0.524 | 0.756 |
| D 3/4 | CD177 + IFNG + S100A9 + CX3CR1 + CIITA + TAP2 + CTLA4 | 0.64 | 0.524 | 0.756 |
| D 3/4 | CD177 + IFNG + S100A9 + CX3CR1 + CTLA4 | 0.641 | 0.526 | 0.757 |
| D 3/4 | CD177 + IFNG + S100A9 + CX3CR1 + TAP2 | 0.646 | 0.528 | 0.763 |
| D 3/4 | CD177 + IFNG + S100A9 + CX3CR1 + TAP2 + CD3D | 0.64 | 0.522 | 0.758 |
| D 3/4 | CD177 + IFNG + S100A9 + CX3CR1 + TAP2 + CD3D + CTLA4 | 0.639 | 0.522 | 0.755 |
| D 3/4 | CD177 + IFNG + S100A9 + CX3CR1 + TAP2 + CTLA4 | 0.643 | 0.527 | 0.759 |
| D 3/4 | CD177 + IFNG + S100A9 + TAP2 | 0.646 | 0.53 | 0.762 |
| D 3/4 | CD177 + IFNG + S100A9 + TAP2 + CD3D | 0.646 | 0.529 | 0.763 |
| D 3/4 | CD177 + IFNG + S100A9 + TAP2 + CD3D + CTLA4 | 0.641 | 0.527 | 0.755 |
| D 3/4 | CD177 + IFNG + S100A9 + TAP2 + CTLA4 | 0.642 | 0.528 | 0.756 |
| D 3/4 | CD177 + IFNG + TAP2 | 0.611 | 0.502 | 0.721 |
| D 3/4 | CD177 + IFNG + TAP2 + CD3D | 0.61 | 0.497 | 0.723 |
| D 3/4 | CD177 + IFNG + TAP2 + CD3D + CTLA4 | 0.607 | 0.49 | 0.723 |
| D 3/4 | CD177 + IFNG + TAP2 + CTLA4 | 0.603 | 0.492 | 0.715 |
| D 3/4 | CD177 + IL1R2 | 0.612 | 0.499 | 0.725 |
| D 3/4 | CD177 + IL1R2 + C3AR1 | 0.624 | 0.515 | 0.734 |
| D 3/4 | CD177 + IL1R2 + C3AR1 + CD3D | 0.628 | 0.515 | 0.741 |
| D 3/4 | CD177 + IL1R2 + C3AR1 + CD3D + CTLA4 | 0.642 | 0.53 | 0.754 |
| D 3/4 | CD177 + IL1R2 + C3AR1 + CD74 | 0.665 | 0.553 | 0.778 |
| D 3/4 | CD177 + IL1R2 + C3AR1 + CD74 + CD3D | 0.661 | 0.548 | 0.774 |
| D 3/4 | CD177 + IL1R2 + C3AR1 + CD74 + CD3D + CTLA4 | 0.664 | 0.55 | 0.778 |
| D 3/4 | CD177 + IL1R2 + C3AR1 + CD74 + CIITA | 0.669 | 0.557 | 0.781 |
| D 3/4 | CD177 + IL1R2 + C3AR1 + CD74 + CIITA + CD3D | 0.67 | 0.557 | 0.783 |
| D 3/4 | CD177 + IL1R2 + C3AR1 + CD74 + CIITA + CD3D + CTLA4 | 0.665 | 0.554 | 0.776 |
| D 3/4 | CD177 + IL1R2 + C3AR1 + CD74 + CIITA + CTLA4 | 0.662 | 0.552 | 0.772 |
| D 3/4 | CD177 + IL1R2 + C3AR1 + CD74 + CIITA + TAP2 | 0.667 | 0.556 | 0.777 |
| D 3/4 | CD177 + IL1R2 + C3AR1 + CD74 + CIITA + TAP2 + CD3D | 0.666 | 0.555 | 0.777 |
| D 3/4 | CD177 + IL1R2 + C3AR1 + CD74 + CIITA + TAP2 + CD3D + CTLA4 | 0.668 | 0.556 | 0.779 |
| D 3/4 | CD177 + IL1R2 + C3AR1 + CD74 + CIITA + TAP2 + CTLA4 | 0.662 | 0.554 | 0.771 |
| D 3/4 | CD177 + IL1R2 + C3AR1 + CD74 + CTLA4 | 0.66 | 0.548 | 0.772 |
| D 3/4 | CD177 + IL1R2 + C3AR1 + CD74 + CX3CR1 | 0.663 | 0.551 | 0.776 |
| D 3/4 | CD177 + IL1R2 + C3AR1 + CD74 + CX3CR1 + CD3D | 0.661 | 0.548 | 0.774 |
| D 3/4 | CD177 + IL1R2 + C3AR1 + CD74 + CX3CR1 + CD3D + CTLA4 | 0.664 | 0.551 | 0.778 |
| D 3/4 | CD177 + IL1R2 + C3AR1 + CD74 + CX3CR1 + CIITA | 0.671 | 0.559 | 0.782 |
| D 3/4 | CD177 + IL1R2 + C3AR1 + CD74 + CX3CR1 + CIITA + CD3D | 0.67 | 0.558 | 0.783 |
| D 3/4 | CD177 + IL1R2 + C3AR1 + CD74 + CX3CR1 + CIITA + CD3D + CTLA4 | 0.664 | 0.553 | 0.775 |
| D 3/4 | CD177 + IL1R2 + C3AR1 + CD74 + CX3CR1 + CIITA + CTLA4 | 0.661 | 0.551 | 0.771 |
| D 3/4 | CD177 + IL1R2 + C3AR1 + CD74 + CX3CR1 + CIITA + TAP2 | 0.664 | 0.553 | 0.775 |
| D 3/4 | CD177 + IL1R2 + C3AR1 + CD74 + CX3CR1 + CIITA + TAP2 + CD3D | 0.666 | 0.555 | 0.778 |
| D 3/4 | CD177 + IL1R2 + C3AR1 + CD74 + CX3CR1 + CIITA + TAP2 + CD3D + CTLA4 | 0.668 | 0.555 | 0.78 |
| D 3/4 | CD177 + IL1R2 + C3AR1 + CD74 + CX3CR1 + CIITA + TAP2 + CTLA4 | 0.662 | 0.554 | 0.771 |
| D 3/4 | CD177 + IL1R2 + C3AR1 + CD74 + CX3CR1 + CTLA4 | 0.661 | 0.549 | 0.773 |
| D 3/4 | CD177 + IL1R2 + C3AR1 + CD74 + CX3CR1 + TAP2 | 0.667 | 0.555 | 0.779 |
| D 3/4 | CD177 + IL1R2 + C3AR1 + CD74 + CX3CR1 + TAP2 + CD3D | 0.665 | 0.553 | 0.778 |
| D 3/4 | CD177 + IL1R2 + C3AR1 + CD74 + CX3CR1 + TAP2 + CD3D + CTLA4 | 0.668 | 0.554 | 0.781 |
| D 3/4 | CD177 + IL1R2 + C3AR1 + CD74 + CX3CR1 + TAP2 + CTLA4 | 0.665 | 0.554 | 0.776 |
| D 3/4 | CD177 + IL1R2 + C3AR1 + CD74 + IFNG | 0.663 | 0.551 | 0.775 |
| D 3/4 | CD177 + IL1R2 + C3AR1 + CD74 + IFNG + CD3D | 0.664 | 0.551 | 0.778 |
| D 3/4 | CD177 + IL1R2 + C3AR1 + CD74 + IFNG + CD3D + CTLA4 | 0.661 | 0.549 | 0.773 |
| D 3/4 | CD177 + IL1R2 + C3AR1 + CD74 + IFNG + CIITA | 0.66 | 0.55 | 0.769 |
| D 3/4 | CD177 + IL1R2 + C3AR1 + CD74 + IFNG + CIITA + CD3D | 0.665 | 0.555 | 0.774 |
| D 3/4 | CD177 + IL1R2 + C3AR1 + CD74 + IFNG + CIITA + CD3D + CTLA4 | 0.664 | 0.556 | 0.773 |
| D 3/4 | CD177 + IL1R2 + C3AR1 + CD74 + IFNG + CIITA + CTLA4 | 0.661 | 0.551 | 0.771 |
| D 3/4 | CD177 + IL1R2 + C3AR1 + CD74 + IFNG + CIITA + TAP2 | 0.665 | 0.556 | 0.774 |
| D 3/4 | CD177 + IL1R2 + C3AR1 + CD74 + IFNG + CIITA + TAP2 + CD3D | 0.662 | 0.554 | 0.77 |
| D 3/4 | CD177 + IL1R2 + C3AR1 + CD74 + IFNG + CIITA + TAP2 + CD3D + CTLA4 | 0.666 | 0.557 | 0.775 |
| D 3/4 | CD177 + IL1R2 + C3AR1 + CD74 + IFNG + CIITA + TAP2 + CTLA4 | 0.661 | 0.552 | 0.771 |
| D 3/4 | CD177 + IL1R2 + C3AR1 + CD74 + IFNG + CTLA4 | 0.661 | 0.551 | 0.772 |
| D 3/4 | CD177 + IL1R2 + C3AR1 + CD74 + IFNG + CX3CR1 | 0.665 | 0.553 | 0.776 |
| D 3/4 | CD177 + IL1R2 + C3AR1 + CD74 + IFNG + CX3CR1 + CD3D | 0.665 | 0.552 | 0.779 |
| D 3/4 | CD177 + IL1R2 + C3AR1 + CD74 + IFNG + CX3CR1 + CD3D + CTLA4 | 0.66 | 0.548 | 0.773 |
| D 3/4 | CD177 + IL1R2 + C3AR1 + CD74 + IFNG + CX3CR1 + CIITA | 0.66 | 0.549 | 0.77 |
| D 3/4 | CD177 + IL1R2 + C3AR1 + CD74 + IFNG + CX3CR1 + CIITA + CD3D | 0.665 | 0.555 | 0.775 |
| D 3/4 | CD177 + IL1R2 + C3AR1 + CD74 + IFNG + CX3CR1 + CIITA + CD3D + CTLA4 | 0.664 | 0.556 | 0.773 |
| D 3/4 | CD177 + IL1R2 + C3AR1 + CD74 + IFNG + CX3CR1 + CIITA + CTLA4 | 0.663 | 0.554 | 0.772 |
| D 3/4 | CD177 + IL1R2 + C3AR1 + CD74 + IFNG + CX3CR1 + CIITA + TAP2 | 0.662 | 0.552 | 0.771 |
| D 3/4 | CD177 + IL1R2 + C3AR1 + CD74 + IFNG + CX3CR1 + CIITA + TAP2 + CD3D | 0.667 | 0.558 | 0.775 |
| D 3/4 | CD177 + IL1R2 + C3AR1 + CD74 + IFNG + CX3CR1 + CIITA + TAP2 + CD3D + CTLA4 | 0.666 | 0.557 | 0.775 |
| D 3/4 | CD177 + IL1R2 + C3AR1 + CD74 + IFNG + CX3CR1 + CIITA + TAP2 + CTLA4 | 0.66 | 0.552 | 0.769 |
| D 3/4 | CD177 + IL1R2 + C3AR1 + CD74 + IFNG + CX3CR1 + CTLA4 | 0.663 | 0.552 | 0.774 |
| D 3/4 | CD177 + IL1R2 + C3AR1 + CD74 + IFNG + CX3CR1 + TAP2 | 0.665 | 0.553 | 0.777 |
| D 3/4 | CD177 + IL1R2 + C3AR1 + CD74 + IFNG + CX3CR1 + TAP2 + CD3D | 0.667 | 0.555 | 0.778 |
| D 3/4 | CD177 + IL1R2 + C3AR1 + CD74 + IFNG + CX3CR1 + TAP2 + CD3D + CTLA4 | 0.664 | 0.552 | 0.776 |
| D 3/4 | CD177 + IL1R2 + C3AR1 + CD74 + IFNG + CX3CR1 + TAP2 + CTLA4 | 0.664 | 0.551 | 0.776 |
| D 3/4 | CD177 + IL1R2 + C3AR1 + CD74 + IFNG + S100A9 | 0.668 | 0.552 | 0.783 |
| D 3/4 | CD177 + IL1R2 + C3AR1 + CD74 + IFNG + S100A9 + CD3D | 0.666 | 0.551 | 0.782 |
| D 3/4 | CD177 + IL1R2 + C3AR1 + CD74 + IFNG + S100A9 + CD3D + CTLA4 | 0.666 | 0.552 | 0.78 |
| D 3/4 | CD177 + IL1R2 + C3AR1 + CD74 + IFNG + S100A9 + CIITA | 0.665 | 0.549 | 0.781 |
| D 3/4 | CD177 + IL1R2 + C3AR1 + CD74 + IFNG + S100A9 + CIITA + CD3D | 0.664 | 0.548 | 0.781 |
| D 3/4 | CD177 + IL1R2 + C3AR1 + CD74 + IFNG + S100A9 + CIITA + CD3D + CTLA4 | 0.663 | 0.546 | 0.779 |
| D 3/4 | CD177 + IL1R2 + C3AR1 + CD74 + IFNG + S100A9 + CIITA + CTLA4 | 0.661 | 0.544 | 0.778 |
| D 3/4 | CD177 + IL1R2 + C3AR1 + CD74 + IFNG + S100A9 + CIITA + TAP2 | 0.667 | 0.551 | 0.783 |
| D 3/4 | CD177 + IL1R2 + C3AR1 + CD74 + IFNG + S100A9 + CIITA + TAP2 + CD3D | 0.668 | 0.552 | 0.784 |
| D 3/4 | CD177 + IL1R2 + C3AR1 + CD74 + IFNG + S100A9 + CIITA + TAP2 + CD3D + CTLA4 | 0.67 | 0.555 | 0.784 |
| D 3/4 | CD177 + IL1R2 + C3AR1 + CD74 + IFNG + S100A9 + CIITA + TAP2 + CTLA4 | 0.665 | 0.548 | 0.781 |
| D 3/4 | CD177 + IL1R2 + C3AR1 + CD74 + IFNG + S100A9 + CTLA4 | 0.666 | 0.551 | 0.782 |
| D 3/4 | CD177 + IL1R2 + C3AR1 + CD74 + IFNG + S100A9 + CX3CR1 | 0.666 | 0.551 | 0.781 |
| D 3/4 | CD177 + IL1R2 + C3AR1 + CD74 + IFNG + S100A9 + CX3CR1 + CD3D | 0.665 | 0.549 | 0.781 |
| D 3/4 | CD177 + IL1R2 + C3AR1 + CD74 + IFNG + S100A9 + CX3CR1 + CD3D + CTLA4 | 0.664 | 0.55 | 0.778 |
| D 3/4 | CD177 + IL1R2 + C3AR1 + CD74 + IFNG + S100A9 + CX3CR1 + CIITA | 0.664 | 0.548 | 0.779 |
| D 3/4 | CD177 + IL1R2 + C3AR1 + CD74 + IFNG + S100A9 + CX3CR1 + CIITA + CD3D | 0.664 | 0.548 | 0.78 |
| D 3/4 | CD177 + IL1R2 + C3AR1 + CD74 + IFNG + S100A9 + CX3CR1 + CIITA + CD3D + CTLA4 | 0.663 | 0.548 | 0.778 |
| D 3/4 | CD177 + IL1R2 + C3AR1 + CD74 + IFNG + S100A9 + CX3CR1 + CIITA + CTLA4 | 0.661 | 0.545 | 0.777 |
| D 3/4 | CD177 + IL1R2 + C3AR1 + CD74 + IFNG + S100A9 + CX3CR1 + CIITA + TAP2 | 0.666 | 0.55 | 0.782 |
| D 3/4 | CD177 + IL1R2 + C3AR1 + CD74 + IFNG + S100A9 + CX3CR1 + CIITA + TAP2 + CD3D | 0.669 | 0.554 | 0.785 |
| D 3/4 | CD177 + IL1R2 + C3AR1 + CD74 + IFNG + S100A9 + CX3CR1 + CIITA + TAP2 + CD3D + CTLA4 | 0.67 | 0.556 | 0.784 |
| D 3/4 | CD177 + IL1R2 + C3AR1 + CD74 + IFNG + S100A9 + CX3CR1 + CIITA + TAP2 + CTLA4 | 0.669 | 0.552 | 0.785 |
| D 3/4 | CD177 + IL1R2 + C3AR1 + CD74 + IFNG + S100A9 + CX3CR1 + CTLA4 | 0.665 | 0.55 | 0.781 |
| D 3/4 | CD177 + IL1R2 + C3AR1 + CD74 + IFNG + S100A9 + CX3CR1 + TAP2 | 0.665 | 0.551 | 0.78 |
| D 3/4 | CD177 + IL1R2 + C3AR1 + CD74 + IFNG + S100A9 + CX3CR1 + TAP2 + CD3D | 0.667 | 0.553 | 0.781 |
| D 3/4 | CD177 + IL1R2 + C3AR1 + CD74 + IFNG + S100A9 + CX3CR1 + TAP2 + CD3D + CTLA4 | 0.667 | 0.553 | 0.781 |
| D 3/4 | CD177 + IL1R2 + C3AR1 + CD74 + IFNG + S100A9 + CX3CR1 + TAP2 + CTLA4 | 0.664 | 0.549 | 0.779 |
| D 3/4 | CD177 + IL1R2 + C3AR1 + CD74 + IFNG + S100A9 + TAP2 | 0.666 | 0.55 | 0.781 |
| D 3/4 | CD177 + IL1R2 + C3AR1 + CD74 + IFNG + S100A9 + TAP2 + CD3D | 0.667 | 0.553 | 0.781 |
| D 3/4 | CD177 + IL1R2 + C3AR1 + CD74 + IFNG + S100A9 + TAP2 + CD3D + CTLA4 | 0.667 | 0.554 | 0.781 |
| D 3/4 | CD177 + IL1R2 + C3AR1 + CD74 + IFNG + S100A9 + TAP2 + CTLA4 | 0.665 | 0.55 | 0.78 |
| D 3/4 | CD177 + IL1R2 + C3AR1 + CD74 + IFNG + TAP2 | 0.665 | 0.555 | 0.775 |
| D 3/4 | CD177 + IL1R2 + C3AR1 + CD74 + IFNG + TAP2 + CD3D | 0.668 | 0.556 | 0.779 |
| D 3/4 | CD177 + IL1R2 + C3AR1 + CD74 + IFNG + TAP2 + CD3D + CTLA4 | 0.666 | 0.554 | 0.778 |
| D 3/4 | CD177 + IL1R2 + C3AR1 + CD74 + IFNG + TAP2 + CTLA4 | 0.663 | 0.552 | 0.774 |
| D 3/4 | CD177 + IL1R2 + C3AR1 + CD74 + S100A9 | 0.664 | 0.548 | 0.78 |
| D 3/4 | CD177 + IL1R2 + C3AR1 + CD74 + S100A9 + CD3D | 0.664 | 0.546 | 0.781 |
| D 3/4 | CD177 + IL1R2 + C3AR1 + CD74 + S100A9 + CD3D + CTLA4 | 0.666 | 0.549 | 0.782 |
| D 3/4 | CD177 + IL1R2 + C3AR1 + CD74 + S100A9 + CIITA | 0.675 | 0.554 | 0.795 |
| D 3/4 | CD177 + IL1R2 + C3AR1 + CD74 + S100A9 + CIITA + CD3D | 0.673 | 0.554 | 0.793 |
| D 3/4 | CD177 + IL1R2 + C3AR1 + CD74 + S100A9 + CIITA + CD3D + CTLA4 | 0.67 | 0.552 | 0.787 |
| D 3/4 | CD177 + IL1R2 + C3AR1 + CD74 + S100A9 + CIITA + CTLA4 | 0.669 | 0.551 | 0.786 |
| D 3/4 | CD177 + IL1R2 + C3AR1 + CD74 + S100A9 + CIITA + TAP2 | 0.671 | 0.553 | 0.789 |
| D 3/4 | CD177 + IL1R2 + C3AR1 + CD74 + S100A9 + CIITA + TAP2 + CD3D | 0.67 | 0.552 | 0.788 |
| D 3/4 | CD177 + IL1R2 + C3AR1 + CD74 + S100A9 + CIITA + TAP2 + CD3D + CTLA4 | 0.671 | 0.553 | 0.789 |
| D 3/4 | CD177 + IL1R2 + C3AR1 + CD74 + S100A9 + CIITA + TAP2 + CTLA4 | 0.668 | 0.551 | 0.785 |
| D 3/4 | CD177 + IL1R2 + C3AR1 + CD74 + S100A9 + CTLA4 | 0.666 | 0.55 | 0.783 |
| D 3/4 | CD177 + IL1R2 + C3AR1 + CD74 + S100A9 + CX3CR1 | 0.665 | 0.548 | 0.781 |
| D 3/4 | CD177 + IL1R2 + C3AR1 + CD74 + S100A9 + CX3CR1 + CD3D | 0.664 | 0.547 | 0.78 |
| D 3/4 | CD177 + IL1R2 + C3AR1 + CD74 + S100A9 + CX3CR1 + CD3D + CTLA4 | 0.666 | 0.55 | 0.783 |
| D 3/4 | CD177 + IL1R2 + C3AR1 + CD74 + S100A9 + CX3CR1 + CIITA | 0.675 | 0.556 | 0.795 |
| D 3/4 | CD177 + IL1R2 + C3AR1 + CD74 + S100A9 + CX3CR1 + CIITA + CD3D | 0.673 | 0.554 | 0.793 |
| D 3/4 | CD177 + IL1R2 + C3AR1 + CD74 + S100A9 + CX3CR1 + CIITA + CD3D + CTLA4 | 0.669 | 0.551 | 0.786 |
| D 3/4 | CD177 + IL1R2 + C3AR1 + CD74 + S100A9 + CX3CR1 + CIITA + CTLA4 | 0.668 | 0.55 | 0.785 |
| D 3/4 | CD177 + IL1R2 + C3AR1 + CD74 + S100A9 + CX3CR1 + CIITA + TAP2 | 0.669 | 0.551 | 0.787 |
| D 3/4 | CD177 + IL1R2 + C3AR1 + CD74 + S100A9 + CX3CR1 + CIITA + TAP2 + CD3D | 0.668 | 0.549 | 0.786 |
| D 3/4 | CD177 + IL1R2 + C3AR1 + CD74 + S100A9 + CX3CR1 + CIITA + TAP2 + CD3D + CTLA4 | 0.671 | 0.553 | 0.789 |
| D 3/4 | CD177 + IL1R2 + C3AR1 + CD74 + S100A9 + CX3CR1 + CIITA + TAP2 + CTLA4 | 0.668 | 0.551 | 0.785 |
| D 3/4 | CD177 + IL1R2 + C3AR1 + CD74 + S100A9 + CX3CR1 + CTLA4 | 0.666 | 0.549 | 0.782 |
| D 3/4 | CD177 + IL1R2 + C3AR1 + CD74 + S100A9 + CX3CR1 + TAP2 | 0.664 | 0.55 | 0.779 |
| D 3/4 | CD177 + IL1R2 + C3AR1 + CD74 + S100A9 + CX3CR1 + TAP2 + CD3D | 0.667 | 0.551 | 0.782 |
| D 3/4 | CD177 + IL1R2 + C3AR1 + CD74 + S100A9 + CX3CR1 + TAP2 + CD3D + CTLA4 | 0.664 | 0.549 | 0.779 |
| D 3/4 | CD177 + IL1R2 + C3AR1 + CD74 + S100A9 + CX3CR1 + TAP2 + CTLA4 | 0.664 | 0.549 | 0.778 |
| D 3/4 | CD177 + IL1R2 + C3AR1 + CD74 + S100A9 + TAP2 | 0.665 | 0.55 | 0.779 |
| D 3/4 | CD177 + IL1R2 + C3AR1 + CD74 + S100A9 + TAP2 + CD3D | 0.664 | 0.549 | 0.78 |
| D 3/4 | CD177 + IL1R2 + C3AR1 + CD74 + S100A9 + TAP2 + CD3D + CTLA4 | 0.664 | 0.549 | 0.779 |
| D 3/4 | CD177 + IL1R2 + C3AR1 + CD74 + S100A9 + TAP2 + CTLA4 | 0.663 | 0.548 | 0.778 |
| D 3/4 | CD177 + IL1R2 + C3AR1 + CD74 + TAP2 | 0.669 | 0.557 | 0.781 |
| D 3/4 | CD177 + IL1R2 + C3AR1 + CD74 + TAP2 + CD3D | 0.667 | 0.554 | 0.78 |
| D 3/4 | CD177 + IL1R2 + C3AR1 + CD74 + TAP2 + CD3D + CTLA4 | 0.667 | 0.554 | 0.78 |
| D 3/4 | CD177 + IL1R2 + C3AR1 + CD74 + TAP2 + CTLA4 | 0.664 | 0.553 | 0.774 |
| D 3/4 | CD177 + IL1R2 + C3AR1 + CIITA | 0.628 | 0.518 | 0.738 |
| D 3/4 | CD177 + IL1R2 + C3AR1 + CIITA + CD3D | 0.629 | 0.515 | 0.742 |
| D 3/4 | CD177 + IL1R2 + C3AR1 + CIITA + CD3D + CTLA4 | 0.636 | 0.523 | 0.749 |
| D 3/4 | CD177 + IL1R2 + C3AR1 + CIITA + CTLA4 | 0.62 | 0.508 | 0.731 |
| D 3/4 | CD177 + IL1R2 + C3AR1 + CIITA + TAP2 | 0.644 | 0.541 | 0.747 |
| D 3/4 | CD177 + IL1R2 + C3AR1 + CIITA + TAP2 + CD3D | 0.647 | 0.537 | 0.757 |
| D 3/4 | CD177 + IL1R2 + C3AR1 + CIITA + TAP2 + CD3D + CTLA4 | 0.636 | 0.519 | 0.753 |
| D 3/4 | CD177 + IL1R2 + C3AR1 + CIITA + TAP2 + CTLA4 | 0.632 | 0.526 | 0.739 |
| D 3/4 | CD177 + IL1R2 + C3AR1 + CTLA4 | 0.625 | 0.516 | 0.733 |
| D 3/4 | CD177 + IL1R2 + C3AR1 + CX3CR1 | 0.634 | 0.519 | 0.749 |
| D 3/4 | CD177 + IL1R2 + C3AR1 + CX3CR1 + CD3D | 0.633 | 0.517 | 0.75 |
| D 3/4 | CD177 + IL1R2 + C3AR1 + CX3CR1 + CD3D + CTLA4 | 0.64 | 0.528 | 0.751 |
| D 3/4 | CD177 + IL1R2 + C3AR1 + CX3CR1 + CIITA | 0.634 | 0.519 | 0.749 |
| D 3/4 | CD177 + IL1R2 + C3AR1 + CX3CR1 + CIITA + CD3D | 0.633 | 0.517 | 0.749 |
| D 3/4 | CD177 + IL1R2 + C3AR1 + CX3CR1 + CIITA + CD3D + CTLA4 | 0.638 | 0.525 | 0.75 |
| D 3/4 | CD177 + IL1R2 + C3AR1 + CX3CR1 + CIITA + CTLA4 | 0.621 | 0.509 | 0.732 |
| D 3/4 | CD177 + IL1R2 + C3AR1 + CX3CR1 + CIITA + TAP2 | 0.637 | 0.528 | 0.747 |
| D 3/4 | CD177 + IL1R2 + C3AR1 + CX3CR1 + CIITA + TAP2 + CD3D | 0.641 | 0.526 | 0.756 |
| D 3/4 | CD177 + IL1R2 + C3AR1 + CX3CR1 + CIITA + TAP2 + CD3D + CTLA4 | 0.635 | 0.518 | 0.753 |
| D 3/4 | CD177 + IL1R2 + C3AR1 + CX3CR1 + CIITA + TAP2 + CTLA4 | 0.63 | 0.522 | 0.738 |
| D 3/4 | CD177 + IL1R2 + C3AR1 + CX3CR1 + CTLA4 | 0.625 | 0.516 | 0.735 |
| D 3/4 | CD177 + IL1R2 + C3AR1 + CX3CR1 + TAP2 | 0.64 | 0.53 | 0.749 |
| D 3/4 | CD177 + IL1R2 + C3AR1 + CX3CR1 + TAP2 + CD3D | 0.648 | 0.533 | 0.762 |
| D 3/4 | CD177 + IL1R2 + C3AR1 + CX3CR1 + TAP2 + CD3D + CTLA4 | 0.648 | 0.532 | 0.764 |
| D 3/4 | CD177 + IL1R2 + C3AR1 + CX3CR1 + TAP2 + CTLA4 | 0.638 | 0.53 | 0.747 |
| D 3/4 | CD177 + IL1R2 + C3AR1 + IFNG | 0.626 | 0.517 | 0.736 |
| D 3/4 | CD177 + IL1R2 + C3AR1 + IFNG + CD3D | 0.628 | 0.515 | 0.74 |
| D 3/4 | CD177 + IL1R2 + C3AR1 + IFNG + CD3D + CTLA4 | 0.64 | 0.527 | 0.752 |
| D 3/4 | CD177 + IL1R2 + C3AR1 + IFNG + CIITA | 0.628 | 0.518 | 0.738 |
| D 3/4 | CD177 + IL1R2 + C3AR1 + IFNG + CIITA + CD3D | 0.627 | 0.514 | 0.74 |
| D 3/4 | CD177 + IL1R2 + C3AR1 + IFNG + CIITA + CD3D + CTLA4 | 0.636 | 0.523 | 0.749 |
| D 3/4 | CD177 + IL1R2 + C3AR1 + IFNG + CIITA + CTLA4 | 0.62 | 0.509 | 0.731 |
| D 3/4 | CD177 + IL1R2 + C3AR1 + IFNG + CIITA + TAP2 | 0.643 | 0.539 | 0.747 |
| D 3/4 | CD177 + IL1R2 + C3AR1 + IFNG + CIITA + TAP2 + CD3D | 0.646 | 0.536 | 0.756 |
| D 3/4 | CD177 + IL1R2 + C3AR1 + IFNG + CIITA + TAP2 + CD3D + CTLA4 | 0.636 | 0.52 | 0.753 |
| D 3/4 | CD177 + IL1R2 + C3AR1 + IFNG + CIITA + TAP2 + CTLA4 | 0.631 | 0.524 | 0.737 |
| D 3/4 | CD177 + IL1R2 + C3AR1 + IFNG + CTLA4 | 0.625 | 0.516 | 0.735 |
| D 3/4 | CD177 + IL1R2 + C3AR1 + IFNG + CX3CR1 | 0.634 | 0.519 | 0.749 |
| D 3/4 | CD177 + IL1R2 + C3AR1 + IFNG + CX3CR1 + CD3D | 0.636 | 0.521 | 0.751 |
| D 3/4 | CD177 + IL1R2 + C3AR1 + IFNG + CX3CR1 + CD3D + CTLA4 | 0.64 | 0.528 | 0.752 |
| D 3/4 | CD177 + IL1R2 + C3AR1 + IFNG + CX3CR1 + CIITA | 0.632 | 0.517 | 0.748 |
| D 3/4 | CD177 + IL1R2 + C3AR1 + IFNG + CX3CR1 + CIITA + CD3D | 0.635 | 0.519 | 0.75 |
| D 3/4 | CD177 + IL1R2 + C3AR1 + IFNG + CX3CR1 + CIITA + CD3D + CTLA4 | 0.636 | 0.524 | 0.749 |
| D 3/4 | CD177 + IL1R2 + C3AR1 + IFNG + CX3CR1 + CIITA + CTLA4 | 0.621 | 0.509 | 0.732 |
| D 3/4 | CD177 + IL1R2 + C3AR1 + IFNG + CX3CR1 + CIITA + TAP2 | 0.634 | 0.524 | 0.744 |
| D 3/4 | CD177 + IL1R2 + C3AR1 + IFNG + CX3CR1 + CIITA + TAP2 + CD3D | 0.641 | 0.527 | 0.756 |
| D 3/4 | CD177 + IL1R2 + C3AR1 + IFNG + CX3CR1 + CIITA + TAP2 + CD3D + CTLA4 | 0.637 | 0.52 | 0.755 |
| D 3/4 | CD177 + IL1R2 + C3AR1 + IFNG + CX3CR1 + CIITA + TAP2 + CTLA4 | 0.629 | 0.521 | 0.738 |
| D 3/4 | CD177 + IL1R2 + C3AR1 + IFNG + CX3CR1 + CTLA4 | 0.627 | 0.518 | 0.736 |
| D 3/4 | CD177 + IL1R2 + C3AR1 + IFNG + CX3CR1 + TAP2 | 0.642 | 0.532 | 0.751 |
| D 3/4 | CD177 + IL1R2 + C3AR1 + IFNG + CX3CR1 + TAP2 + CD3D | 0.651 | 0.537 | 0.765 |
| D 3/4 | CD177 + IL1R2 + C3AR1 + IFNG + CX3CR1 + TAP2 + CD3D + CTLA4 | 0.643 | 0.526 | 0.759 |
| D 3/4 | CD177 + IL1R2 + C3AR1 + IFNG + CX3CR1 + TAP2 + CTLA4 | 0.635 | 0.526 | 0.745 |
| D 3/4 | CD177 + IL1R2 + C3AR1 + IFNG + S100A9 | 0.651 | 0.537 | 0.765 |
| D 3/4 | CD177 + IL1R2 + C3AR1 + IFNG + S100A9 + CD3D | 0.65 | 0.536 | 0.764 |
| D 3/4 | CD177 + IL1R2 + C3AR1 + IFNG + S100A9 + CD3D + CTLA4 | 0.643 | 0.531 | 0.754 |
| D 3/4 | CD177 + IL1R2 + C3AR1 + IFNG + S100A9 + CIITA | 0.644 | 0.529 | 0.76 |
| D 3/4 | CD177 + IL1R2 + C3AR1 + IFNG + S100A9 + CIITA + CD3D | 0.64 | 0.525 | 0.755 |
| D 3/4 | CD177 + IL1R2 + C3AR1 + IFNG + S100A9 + CIITA + CD3D + CTLA4 | 0.635 | 0.521 | 0.748 |
| D 3/4 | CD177 + IL1R2 + C3AR1 + IFNG + S100A9 + CIITA + CTLA4 | 0.632 | 0.52 | 0.744 |
| D 3/4 | CD177 + IL1R2 + C3AR1 + IFNG + S100A9 + CIITA + TAP2 | 0.645 | 0.533 | 0.756 |
| D 3/4 | CD177 + IL1R2 + C3AR1 + IFNG + S100A9 + CIITA + TAP2 + CD3D | 0.644 | 0.532 | 0.757 |
| D 3/4 | CD177 + IL1R2 + C3AR1 + IFNG + S100A9 + CIITA + TAP2 + CD3D + CTLA4 | 0.644 | 0.529 | 0.759 |
| D 3/4 | CD177 + IL1R2 + C3AR1 + IFNG + S100A9 + CIITA + TAP2 + CTLA4 | 0.637 | 0.526 | 0.747 |
| D 3/4 | CD177 + IL1R2 + C3AR1 + IFNG + S100A9 + CTLA4 | 0.644 | 0.534 | 0.754 |
| D 3/4 | CD177 + IL1R2 + C3AR1 + IFNG + S100A9 + CX3CR1 | 0.651 | 0.536 | 0.766 |
| D 3/4 | CD177 + IL1R2 + C3AR1 + IFNG + S100A9 + CX3CR1 + CD3D | 0.646 | 0.531 | 0.761 |
| D 3/4 | CD177 + IL1R2 + C3AR1 + IFNG + S100A9 + CX3CR1 + CD3D + CTLA4 | 0.643 | 0.531 | 0.755 |
| D 3/4 | CD177 + IL1R2 + C3AR1 + IFNG + S100A9 + CX3CR1 + CIITA | 0.647 | 0.53 | 0.764 |
| D 3/4 | CD177 + IL1R2 + C3AR1 + IFNG + S100A9 + CX3CR1 + CIITA + CD3D | 0.639 | 0.524 | 0.754 |
| D 3/4 | CD177 + IL1R2 + C3AR1 + IFNG + S100A9 + CX3CR1 + CIITA + CD3D + CTLA4 | 0.635 | 0.522 | 0.748 |
| D 3/4 | CD177 + IL1R2 + C3AR1 + IFNG + S100A9 + CX3CR1 + CIITA + CTLA4 | 0.629 | 0.516 | 0.741 |
| D 3/4 | CD177 + IL1R2 + C3AR1 + IFNG + S100A9 + CX3CR1 + CIITA + TAP2 | 0.646 | 0.532 | 0.76 |
| D 3/4 | CD177 + IL1R2 + C3AR1 + IFNG + S100A9 + CX3CR1 + CIITA + TAP2 + CD3D | 0.645 | 0.531 | 0.759 |
| D 3/4 | CD177 + IL1R2 + C3AR1 + IFNG + S100A9 + CX3CR1 + CIITA + TAP2 + CD3D + CTLA4 | 0.643 | 0.529 | 0.758 |
| D 3/4 | CD177 + IL1R2 + C3AR1 + IFNG + S100A9 + CX3CR1 + CIITA + TAP2 + CTLA4 | 0.64 | 0.53 | 0.75 |
| D 3/4 | CD177 + IL1R2 + C3AR1 + IFNG + S100A9 + CX3CR1 + CTLA4 | 0.641 | 0.53 | 0.753 |
| D 3/4 | CD177 + IL1R2 + C3AR1 + IFNG + S100A9 + CX3CR1 + TAP2 | 0.651 | 0.538 | 0.763 |
| D 3/4 | CD177 + IL1R2 + C3AR1 + IFNG + S100A9 + CX3CR1 + TAP2 + CD3D | 0.654 | 0.54 | 0.768 |
| D 3/4 | CD177 + IL1R2 + C3AR1 + IFNG + S100A9 + CX3CR1 + TAP2 + CD3D + CTLA4 | 0.652 | 0.538 | 0.766 |
| D 3/4 | CD177 + IL1R2 + C3AR1 + IFNG + S100A9 + CX3CR1 + TAP2 + CTLA4 | 0.646 | 0.534 | 0.758 |
| D 3/4 | CD177 + IL1R2 + C3AR1 + IFNG + S100A9 + TAP2 | 0.65 | 0.537 | 0.764 |
| D 3/4 | CD177 + IL1R2 + C3AR1 + IFNG + S100A9 + TAP2 + CD3D | 0.653 | 0.539 | 0.766 |
| D 3/4 | CD177 + IL1R2 + C3AR1 + IFNG + S100A9 + TAP2 + CD3D + CTLA4 | 0.649 | 0.535 | 0.764 |
| D 3/4 | CD177 + IL1R2 + C3AR1 + IFNG + S100A9 + TAP2 + CTLA4 | 0.646 | 0.535 | 0.757 |
| D 3/4 | CD177 + IL1R2 + C3AR1 + IFNG + TAP2 | 0.639 | 0.534 | 0.744 |
| D 3/4 | CD177 + IL1R2 + C3AR1 + IFNG + TAP2 + CD3D | 0.654 | 0.543 | 0.765 |
| D 3/4 | CD177 + IL1R2 + C3AR1 + IFNG + TAP2 + CD3D + CTLA4 | 0.644 | 0.528 | 0.761 |
| D 3/4 | CD177 + IL1R2 + C3AR1 + IFNG + TAP2 + CTLA4 | 0.636 | 0.528 | 0.743 |
| D 3/4 | CD177 + IL1R2 + C3AR1 + S100A9 | 0.651 | 0.539 | 0.764 |
| D 3/4 | CD177 + IL1R2 + C3AR1 + S100A9 + CD3D | 0.649 | 0.536 | 0.762 |
| D 3/4 | CD177 + IL1R2 + C3AR1 + S100A9 + CD3D + CTLA4 | 0.646 | 0.536 | 0.756 |
| D 3/4 | CD177 + IL1R2 + C3AR1 + S100A9 + CIITA | 0.645 | 0.529 | 0.76 |
| D 3/4 | CD177 + IL1R2 + C3AR1 + S100A9 + CIITA + CD3D | 0.64 | 0.525 | 0.755 |
| D 3/4 | CD177 + IL1R2 + C3AR1 + S100A9 + CIITA + CD3D + CTLA4 | 0.632 | 0.52 | 0.745 |
| D 3/4 | CD177 + IL1R2 + C3AR1 + S100A9 + CIITA + CTLA4 | 0.63 | 0.517 | 0.742 |
| D 3/4 | CD177 + IL1R2 + C3AR1 + S100A9 + CIITA + TAP2 | 0.643 | 0.533 | 0.754 |
| D 3/4 | CD177 + IL1R2 + C3AR1 + S100A9 + CIITA + TAP2 + CD3D | 0.647 | 0.536 | 0.759 |
| D 3/4 | CD177 + IL1R2 + C3AR1 + S100A9 + CIITA + TAP2 + CD3D + CTLA4 | 0.647 | 0.534 | 0.76 |
| D 3/4 | CD177 + IL1R2 + C3AR1 + S100A9 + CIITA + TAP2 + CTLA4 | 0.639 | 0.53 | 0.748 |
| D 3/4 | CD177 + IL1R2 + C3AR1 + S100A9 + CTLA4 | 0.647 | 0.539 | 0.756 |
| D 3/4 | CD177 + IL1R2 + C3AR1 + S100A9 + CX3CR1 | 0.651 | 0.535 | 0.766 |
| D 3/4 | CD177 + IL1R2 + C3AR1 + S100A9 + CX3CR1 + CD3D | 0.646 | 0.532 | 0.761 |
| D 3/4 | CD177 + IL1R2 + C3AR1 + S100A9 + CX3CR1 + CD3D + CTLA4 | 0.644 | 0.533 | 0.754 |
| D 3/4 | CD177 + IL1R2 + C3AR1 + S100A9 + CX3CR1 + CIITA | 0.649 | 0.532 | 0.766 |
| D 3/4 | CD177 + IL1R2 + C3AR1 + S100A9 + CX3CR1 + CIITA + CD3D | 0.639 | 0.524 | 0.755 |
| D 3/4 | CD177 + IL1R2 + C3AR1 + S100A9 + CX3CR1 + CIITA + CD3D + CTLA4 | 0.634 | 0.521 | 0.747 |
| D 3/4 | CD177 + IL1R2 + C3AR1 + S100A9 + CX3CR1 + CIITA + CTLA4 | 0.627 | 0.513 | 0.741 |
| D 3/4 | CD177 + IL1R2 + C3AR1 + S100A9 + CX3CR1 + CIITA + TAP2 | 0.646 | 0.533 | 0.76 |
| D 3/4 | CD177 + IL1R2 + C3AR1 + S100A9 + CX3CR1 + CIITA + TAP2 + CD3D | 0.645 | 0.53 | 0.759 |
| D 3/4 | CD177 + IL1R2 + C3AR1 + S100A9 + CX3CR1 + CIITA + TAP2 + CD3D + CTLA4 | 0.643 | 0.529 | 0.757 |
| D 3/4 | CD177 + IL1R2 + C3AR1 + S100A9 + CX3CR1 + CIITA + TAP2 + CTLA4 | 0.637 | 0.527 | 0.747 |
| D 3/4 | CD177 + IL1R2 + C3AR1 + S100A9 + CX3CR1 + CTLA4 | 0.646 | 0.535 | 0.756 |
| D 3/4 | CD177 + IL1R2 + C3AR1 + S100A9 + CX3CR1 + TAP2 | 0.657 | 0.545 | 0.77 |
| D 3/4 | CD177 + IL1R2 + C3AR1 + S100A9 + CX3CR1 + TAP2 + CD3D | 0.657 | 0.544 | 0.77 |
| D 3/4 | CD177 + IL1R2 + C3AR1 + S100A9 + CX3CR1 + TAP2 + CD3D + CTLA4 | 0.655 | 0.542 | 0.767 |
| D 3/4 | CD177 + IL1R2 + C3AR1 + S100A9 + CX3CR1 + TAP2 + CTLA4 | 0.657 | 0.546 | 0.768 |
| D 3/4 | CD177 + IL1R2 + C3AR1 + S100A9 + TAP2 | 0.649 | 0.54 | 0.759 |
| D 3/4 | CD177 + IL1R2 + C3AR1 + S100A9 + TAP2 + CD3D | 0.656 | 0.544 | 0.768 |
| D 3/4 | CD177 + IL1R2 + C3AR1 + S100A9 + TAP2 + CD3D + CTLA4 | 0.652 | 0.539 | 0.765 |
| D 3/4 | CD177 + IL1R2 + C3AR1 + S100A9 + TAP2 + CTLA4 | 0.653 | 0.545 | 0.76 |
| D 3/4 | CD177 + IL1R2 + C3AR1 + TAP2 | 0.65 | 0.547 | 0.753 |
| D 3/4 | CD177 + IL1R2 + C3AR1 + TAP2 + CD3D | 0.655 | 0.545 | 0.765 |
| D 3/4 | CD177 + IL1R2 + C3AR1 + TAP2 + CD3D + CTLA4 | 0.647 | 0.532 | 0.762 |
| D 3/4 | CD177 + IL1R2 + C3AR1 + TAP2 + CTLA4 | 0.638 | 0.533 | 0.744 |
| D 3/4 | CD177 + IL1R2 + CD3D | 0.615 | 0.499 | 0.73 |
| D 3/4 | CD177 + IL1R2 + CD3D + CTLA4 | 0.623 | 0.505 | 0.74 |
| D 3/4 | CD177 + IL1R2 + CD74 | 0.635 | 0.521 | 0.75 |
| D 3/4 | CD177 + IL1R2 + CD74 + CD3D | 0.632 | 0.517 | 0.747 |
| D 3/4 | CD177 + IL1R2 + CD74 + CD3D + CTLA4 | 0.632 | 0.517 | 0.748 |
| D 3/4 | CD177 + IL1R2 + CD74 + CIITA | 0.64 | 0.526 | 0.753 |
| D 3/4 | CD177 + IL1R2 + CD74 + CIITA + CD3D | 0.639 | 0.525 | 0.753 |
| D 3/4 | CD177 + IL1R2 + CD74 + CIITA + CD3D + CTLA4 | 0.64 | 0.525 | 0.755 |
| D 3/4 | CD177 + IL1R2 + CD74 + CIITA + CTLA4 | 0.639 | 0.525 | 0.753 |
| D 3/4 | CD177 + IL1R2 + CD74 + CIITA + TAP2 | 0.645 | 0.531 | 0.758 |
| D 3/4 | CD177 + IL1R2 + CD74 + CIITA + TAP2 + CD3D | 0.642 | 0.525 | 0.759 |
| D 3/4 | CD177 + IL1R2 + CD74 + CIITA + TAP2 + CD3D + CTLA4 | 0.642 | 0.524 | 0.759 |
| D 3/4 | CD177 + IL1R2 + CD74 + CIITA + TAP2 + CTLA4 | 0.645 | 0.531 | 0.758 |
| D 3/4 | CD177 + IL1R2 + CD74 + CTLA4 | 0.636 | 0.521 | 0.751 |
| D 3/4 | CD177 + IL1R2 + CD74 + CX3CR1 | 0.631 | 0.516 | 0.747 |
| D 3/4 | CD177 + IL1R2 + CD74 + CX3CR1 + CD3D | 0.632 | 0.516 | 0.748 |
| D 3/4 | CD177 + IL1R2 + CD74 + CX3CR1 + CD3D + CTLA4 | 0.631 | 0.515 | 0.748 |
| D 3/4 | CD177 + IL1R2 + CD74 + CX3CR1 + CIITA | 0.638 | 0.524 | 0.752 |
| D 3/4 | CD177 + IL1R2 + CD74 + CX3CR1 + CIITA + CD3D | 0.638 | 0.524 | 0.752 |
| D 3/4 | CD177 + IL1R2 + CD74 + CX3CR1 + CIITA + CD3D + CTLA4 | 0.64 | 0.526 | 0.754 |
| D 3/4 | CD177 + IL1R2 + CD74 + CX3CR1 + CIITA + CTLA4 | 0.641 | 0.527 | 0.754 |
| D 3/4 | CD177 + IL1R2 + CD74 + CX3CR1 + CIITA + TAP2 | 0.642 | 0.526 | 0.758 |
| D 3/4 | CD177 + IL1R2 + CD74 + CX3CR1 + CIITA + TAP2 + CD3D | 0.643 | 0.526 | 0.761 |
| D 3/4 | CD177 + IL1R2 + CD74 + CX3CR1 + CIITA + TAP2 + CD3D + CTLA4 | 0.646 | 0.528 | 0.763 |
| D 3/4 | CD177 + IL1R2 + CD74 + CX3CR1 + CIITA + TAP2 + CTLA4 | 0.643 | 0.527 | 0.758 |
| D 3/4 | CD177 + IL1R2 + CD74 + CX3CR1 + CTLA4 | 0.63 | 0.515 | 0.746 |
| D 3/4 | CD177 + IL1R2 + CD74 + CX3CR1 + TAP2 | 0.636 | 0.521 | 0.751 |
| D 3/4 | CD177 + IL1R2 + CD74 + CX3CR1 + TAP2 + CD3D | 0.632 | 0.514 | 0.749 |
| D 3/4 | CD177 + IL1R2 + CD74 + CX3CR1 + TAP2 + CD3D + CTLA4 | 0.633 | 0.515 | 0.75 |
| D 3/4 | CD177 + IL1R2 + CD74 + CX3CR1 + TAP2 + CTLA4 | 0.634 | 0.518 | 0.749 |
| D 3/4 | CD177 + IL1R2 + CD74 + IFNG | 0.637 | 0.525 | 0.75 |
| D 3/4 | CD177 + IL1R2 + CD74 + IFNG + CD3D | 0.635 | 0.52 | 0.751 |
| D 3/4 | CD177 + IL1R2 + CD74 + IFNG + CD3D + CTLA4 | 0.634 | 0.519 | 0.75 |
| D 3/4 | CD177 + IL1R2 + CD74 + IFNG + CIITA | 0.645 | 0.533 | 0.756 |
| D 3/4 | CD177 + IL1R2 + CD74 + IFNG + CIITA + CD3D | 0.652 | 0.54 | 0.765 |
| D 3/4 | CD177 + IL1R2 + CD74 + IFNG + CIITA + CD3D + CTLA4 | 0.652 | 0.54 | 0.763 |
| D 3/4 | CD177 + IL1R2 + CD74 + IFNG + CIITA + CTLA4 | 0.643 | 0.531 | 0.754 |
| D 3/4 | CD177 + IL1R2 + CD74 + IFNG + CIITA + TAP2 | 0.646 | 0.534 | 0.758 |
| D 3/4 | CD177 + IL1R2 + CD74 + IFNG + CIITA + TAP2 + CD3D | 0.646 | 0.533 | 0.759 |
| D 3/4 | CD177 + IL1R2 + CD74 + IFNG + CIITA + TAP2 + CD3D + CTLA4 | 0.646 | 0.533 | 0.759 |
| D 3/4 | CD177 + IL1R2 + CD74 + IFNG + CIITA + TAP2 + CTLA4 | 0.647 | 0.535 | 0.76 |
| D 3/4 | CD177 + IL1R2 + CD74 + IFNG + CTLA4 | 0.637 | 0.524 | 0.751 |
| D 3/4 | CD177 + IL1R2 + CD74 + IFNG + CX3CR1 | 0.636 | 0.522 | 0.749 |
| D 3/4 | CD177 + IL1R2 + CD74 + IFNG + CX3CR1 + CD3D | 0.634 | 0.519 | 0.749 |
| D 3/4 | CD177 + IL1R2 + CD74 + IFNG + CX3CR1 + CD3D + CTLA4 | 0.634 | 0.519 | 0.749 |
| D 3/4 | CD177 + IL1R2 + CD74 + IFNG + CX3CR1 + CIITA | 0.644 | 0.533 | 0.755 |
| D 3/4 | CD177 + IL1R2 + CD74 + IFNG + CX3CR1 + CIITA + CD3D | 0.649 | 0.537 | 0.761 |
| D 3/4 | CD177 + IL1R2 + CD74 + IFNG + CX3CR1 + CIITA + CD3D + CTLA4 | 0.646 | 0.535 | 0.757 |
| D 3/4 | CD177 + IL1R2 + CD74 + IFNG + CX3CR1 + CIITA + CTLA4 | 0.642 | 0.531 | 0.753 |
| D 3/4 | CD177 + IL1R2 + CD74 + IFNG + CX3CR1 + CIITA + TAP2 | 0.645 | 0.533 | 0.757 |
| D 3/4 | CD177 + IL1R2 + CD74 + IFNG + CX3CR1 + CIITA + TAP2 + CD3D | 0.648 | 0.534 | 0.763 |
| D 3/4 | CD177 + IL1R2 + CD74 + IFNG + CX3CR1 + CIITA + TAP2 + CD3D + CTLA4 | 0.646 | 0.532 | 0.759 |
| D 3/4 | CD177 + IL1R2 + CD74 + IFNG + CX3CR1 + CIITA + TAP2 + CTLA4 | 0.645 | 0.532 | 0.757 |
| D 3/4 | CD177 + IL1R2 + CD74 + IFNG + CX3CR1 + CTLA4 | 0.632 | 0.518 | 0.747 |
| D 3/4 | CD177 + IL1R2 + CD74 + IFNG + CX3CR1 + TAP2 | 0.637 | 0.525 | 0.75 |
| D 3/4 | CD177 + IL1R2 + CD74 + IFNG + CX3CR1 + TAP2 + CD3D | 0.637 | 0.522 | 0.751 |
| D 3/4 | CD177 + IL1R2 + CD74 + IFNG + CX3CR1 + TAP2 + CD3D + CTLA4 | 0.636 | 0.522 | 0.751 |
| D 3/4 | CD177 + IL1R2 + CD74 + IFNG + CX3CR1 + TAP2 + CTLA4 | 0.637 | 0.524 | 0.751 |
| D 3/4 | CD177 + IL1R2 + CD74 + IFNG + S100A9 | 0.646 | 0.53 | 0.763 |
| D 3/4 | CD177 + IL1R2 + CD74 + IFNG + S100A9 + CD3D | 0.648 | 0.532 | 0.764 |
| D 3/4 | CD177 + IL1R2 + CD74 + IFNG + S100A9 + CD3D + CTLA4 | 0.648 | 0.532 | 0.764 |
| D 3/4 | CD177 + IL1R2 + CD74 + IFNG + S100A9 + CIITA | 0.653 | 0.534 | 0.771 |
| D 3/4 | CD177 + IL1R2 + CD74 + IFNG + S100A9 + CIITA + CD3D | 0.652 | 0.533 | 0.77 |
| D 3/4 | CD177 + IL1R2 + CD74 + IFNG + S100A9 + CIITA + CD3D + CTLA4 | 0.651 | 0.531 | 0.77 |
| D 3/4 | CD177 + IL1R2 + CD74 + IFNG + S100A9 + CIITA + CTLA4 | 0.65 | 0.53 | 0.769 |
| D 3/4 | CD177 + IL1R2 + CD74 + IFNG + S100A9 + CIITA + TAP2 | 0.655 | 0.537 | 0.773 |
| D 3/4 | CD177 + IL1R2 + CD74 + IFNG + S100A9 + CIITA + TAP2 + CD3D | 0.651 | 0.533 | 0.769 |
| D 3/4 | CD177 + IL1R2 + CD74 + IFNG + S100A9 + CIITA + TAP2 + CD3D + CTLA4 | 0.653 | 0.535 | 0.772 |
| D 3/4 | CD177 + IL1R2 + CD74 + IFNG + S100A9 + CIITA + TAP2 + CTLA4 | 0.656 | 0.537 | 0.774 |
| D 3/4 | CD177 + IL1R2 + CD74 + IFNG + S100A9 + CTLA4 | 0.646 | 0.529 | 0.763 |
| D 3/4 | CD177 + IL1R2 + CD74 + IFNG + S100A9 + CX3CR1 | 0.643 | 0.526 | 0.761 |
| D 3/4 | CD177 + IL1R2 + CD74 + IFNG + S100A9 + CX3CR1 + CD3D | 0.644 | 0.527 | 0.76 |
| D 3/4 | CD177 + IL1R2 + CD74 + IFNG + S100A9 + CX3CR1 + CD3D + CTLA4 | 0.643 | 0.526 | 0.761 |
| D 3/4 | CD177 + IL1R2 + CD74 + IFNG + S100A9 + CX3CR1 + CIITA | 0.653 | 0.535 | 0.772 |
| D 3/4 | CD177 + IL1R2 + CD74 + IFNG + S100A9 + CX3CR1 + CIITA + CD3D | 0.652 | 0.534 | 0.771 |
| D 3/4 | CD177 + IL1R2 + CD74 + IFNG + S100A9 + CX3CR1 + CIITA + CD3D + CTLA4 | 0.651 | 0.533 | 0.77 |
| D 3/4 | CD177 + IL1R2 + CD74 + IFNG + S100A9 + CX3CR1 + CIITA + CTLA4 | 0.651 | 0.532 | 0.769 |
| D 3/4 | CD177 + IL1R2 + CD74 + IFNG + S100A9 + CX3CR1 + CIITA + TAP2 | 0.654 | 0.537 | 0.772 |
| D 3/4 | CD177 + IL1R2 + CD74 + IFNG + S100A9 + CX3CR1 + CIITA + TAP2 + CD3D | 0.653 | 0.535 | 0.772 |
| D 3/4 | CD177 + IL1R2 + CD74 + IFNG + S100A9 + CX3CR1 + CIITA + TAP2 + CD3D + CTLA4 | 0.654 | 0.536 | 0.773 |
| D 3/4 | CD177 + IL1R2 + CD74 + IFNG + S100A9 + CX3CR1 + CIITA + TAP2 + CTLA4 | 0.654 | 0.536 | 0.772 |
| D 3/4 | CD177 + IL1R2 + CD74 + IFNG + S100A9 + CX3CR1 + CTLA4 | 0.643 | 0.526 | 0.76 |
| D 3/4 | CD177 + IL1R2 + CD74 + IFNG + S100A9 + CX3CR1 + TAP2 | 0.647 | 0.531 | 0.764 |
| D 3/4 | CD177 + IL1R2 + CD74 + IFNG + S100A9 + CX3CR1 + TAP2 + CD3D | 0.644 | 0.527 | 0.761 |
| D 3/4 | CD177 + IL1R2 + CD74 + IFNG + S100A9 + CX3CR1 + TAP2 + CD3D + CTLA4 | 0.644 | 0.526 | 0.762 |
| D 3/4 | CD177 + IL1R2 + CD74 + IFNG + S100A9 + CX3CR1 + TAP2 + CTLA4 | 0.645 | 0.528 | 0.762 |
| D 3/4 | CD177 + IL1R2 + CD74 + IFNG + S100A9 + TAP2 | 0.646 | 0.529 | 0.763 |
| D 3/4 | CD177 + IL1R2 + CD74 + IFNG + S100A9 + TAP2 + CD3D | 0.646 | 0.53 | 0.762 |
| D 3/4 | CD177 + IL1R2 + CD74 + IFNG + S100A9 + TAP2 + CD3D + CTLA4 | 0.646 | 0.53 | 0.763 |
| D 3/4 | CD177 + IL1R2 + CD74 + IFNG + S100A9 + TAP2 + CTLA4 | 0.646 | 0.529 | 0.763 |
| D 3/4 | CD177 + IL1R2 + CD74 + IFNG + TAP2 | 0.642 | 0.53 | 0.754 |
| D 3/4 | CD177 + IL1R2 + CD74 + IFNG + TAP2 + CD3D | 0.636 | 0.521 | 0.75 |
| D 3/4 | CD177 + IL1R2 + CD74 + IFNG + TAP2 + CD3D + CTLA4 | 0.635 | 0.521 | 0.749 |
| D 3/4 | CD177 + IL1R2 + CD74 + IFNG + TAP2 + CTLA4 | 0.64 | 0.527 | 0.753 |
| D 3/4 | CD177 + IL1R2 + CD74 + S100A9 | 0.647 | 0.53 | 0.765 |
| D 3/4 | CD177 + IL1R2 + CD74 + S100A9 + CD3D | 0.646 | 0.528 | 0.764 |
| D 3/4 | CD177 + IL1R2 + CD74 + S100A9 + CD3D + CTLA4 | 0.647 | 0.529 | 0.765 |
| D 3/4 | CD177 + IL1R2 + CD74 + S100A9 + CIITA | 0.652 | 0.529 | 0.776 |
| D 3/4 | CD177 + IL1R2 + CD74 + S100A9 + CIITA + CD3D | 0.656 | 0.532 | 0.779 |
| D 3/4 | CD177 + IL1R2 + CD74 + S100A9 + CIITA + CD3D + CTLA4 | 0.648 | 0.526 | 0.771 |
| D 3/4 | CD177 + IL1R2 + CD74 + S100A9 + CIITA + CTLA4 | 0.647 | 0.525 | 0.769 |
| D 3/4 | CD177 + IL1R2 + CD74 + S100A9 + CIITA + TAP2 | 0.656 | 0.533 | 0.778 |
| D 3/4 | CD177 + IL1R2 + CD74 + S100A9 + CIITA + TAP2 + CD3D | 0.656 | 0.533 | 0.779 |
| D 3/4 | CD177 + IL1R2 + CD74 + S100A9 + CIITA + TAP2 + CD3D + CTLA4 | 0.653 | 0.531 | 0.776 |
| D 3/4 | CD177 + IL1R2 + CD74 + S100A9 + CIITA + TAP2 + CTLA4 | 0.652 | 0.529 | 0.775 |
| D 3/4 | CD177 + IL1R2 + CD74 + S100A9 + CTLA4 | 0.645 | 0.527 | 0.763 |
| D 3/4 | CD177 + IL1R2 + CD74 + S100A9 + CX3CR1 | 0.643 | 0.524 | 0.763 |
| D 3/4 | CD177 + IL1R2 + CD74 + S100A9 + CX3CR1 + CD3D | 0.644 | 0.525 | 0.763 |
| D 3/4 | CD177 + IL1R2 + CD74 + S100A9 + CX3CR1 + CD3D + CTLA4 | 0.645 | 0.525 | 0.764 |
| D 3/4 | CD177 + IL1R2 + CD74 + S100A9 + CX3CR1 + CIITA | 0.652 | 0.528 | 0.775 |
| D 3/4 | CD177 + IL1R2 + CD74 + S100A9 + CX3CR1 + CIITA + CD3D | 0.652 | 0.529 | 0.775 |
| D 3/4 | CD177 + IL1R2 + CD74 + S100A9 + CX3CR1 + CIITA + CD3D + CTLA4 | 0.648 | 0.526 | 0.771 |
| D 3/4 | CD177 + IL1R2 + CD74 + S100A9 + CX3CR1 + CIITA + CTLA4 | 0.645 | 0.523 | 0.766 |
| D 3/4 | CD177 + IL1R2 + CD74 + S100A9 + CX3CR1 + CIITA + TAP2 | 0.656 | 0.532 | 0.779 |
| D 3/4 | CD177 + IL1R2 + CD74 + S100A9 + CX3CR1 + CIITA + TAP2 + CD3D | 0.655 | 0.531 | 0.779 |
| D 3/4 | CD177 + IL1R2 + CD74 + S100A9 + CX3CR1 + CIITA + TAP2 + CD3D + CTLA4 | 0.653 | 0.53 | 0.776 |
| D 3/4 | CD177 + IL1R2 + CD74 + S100A9 + CX3CR1 + CIITA + TAP2 + CTLA4 | 0.653 | 0.53 | 0.777 |
| D 3/4 | CD177 + IL1R2 + CD74 + S100A9 + CX3CR1 + CTLA4 | 0.643 | 0.524 | 0.762 |
| D 3/4 | CD177 + IL1R2 + CD74 + S100A9 + CX3CR1 + TAP2 | 0.644 | 0.526 | 0.763 |
| D 3/4 | CD177 + IL1R2 + CD74 + S100A9 + CX3CR1 + TAP2 + CD3D | 0.643 | 0.525 | 0.762 |
| D 3/4 | CD177 + IL1R2 + CD74 + S100A9 + CX3CR1 + TAP2 + CD3D + CTLA4 | 0.645 | 0.526 | 0.764 |
| D 3/4 | CD177 + IL1R2 + CD74 + S100A9 + CX3CR1 + TAP2 + CTLA4 | 0.644 | 0.524 | 0.763 |
| D 3/4 | CD177 + IL1R2 + CD74 + S100A9 + TAP2 | 0.646 | 0.529 | 0.764 |
| D 3/4 | CD177 + IL1R2 + CD74 + S100A9 + TAP2 + CD3D | 0.642 | 0.525 | 0.759 |
| D 3/4 | CD177 + IL1R2 + CD74 + S100A9 + TAP2 + CD3D + CTLA4 | 0.643 | 0.526 | 0.76 |
| D 3/4 | CD177 + IL1R2 + CD74 + S100A9 + TAP2 + CTLA4 | 0.643 | 0.526 | 0.761 |
| D 3/4 | CD177 + IL1R2 + CD74 + TAP2 | 0.641 | 0.528 | 0.753 |
| D 3/4 | CD177 + IL1R2 + CD74 + TAP2 + CD3D | 0.635 | 0.518 | 0.751 |
| D 3/4 | CD177 + IL1R2 + CD74 + TAP2 + CD3D + CTLA4 | 0.635 | 0.519 | 0.752 |
| D 3/4 | CD177 + IL1R2 + CD74 + TAP2 + CTLA4 | 0.638 | 0.524 | 0.753 |
| D 3/4 | CD177 + IL1R2 + CIITA | 0.609 | 0.494 | 0.724 |
| D 3/4 | CD177 + IL1R2 + CIITA + CD3D | 0.611 | 0.494 | 0.727 |
| D 3/4 | CD177 + IL1R2 + CIITA + CD3D + CTLA4 | 0.613 | 0.494 | 0.733 |
| D 3/4 | CD177 + IL1R2 + CIITA + CTLA4 | 0.606 | 0.488 | 0.724 |
| D 3/4 | CD177 + IL1R2 + CIITA + TAP2 | 0.622 | 0.513 | 0.731 |
| D 3/4 | CD177 + IL1R2 + CIITA + TAP2 + CD3D | 0.628 | 0.513 | 0.742 |
| D 3/4 | CD177 + IL1R2 + CIITA + TAP2 + CD3D + CTLA4 | 0.619 | 0.498 | 0.741 |
| D 3/4 | CD177 + IL1R2 + CIITA + TAP2 + CTLA4 | 0.616 | 0.503 | 0.729 |
| D 3/4 | CD177 + IL1R2 + CTLA4 | 0.614 | 0.499 | 0.73 |
| D 3/4 | CD177 + IL1R2 + CX3CR1 | 0.615 | 0.497 | 0.733 |
| D 3/4 | CD177 + IL1R2 + CX3CR1 + CD3D | 0.616 | 0.497 | 0.734 |
| D 3/4 | CD177 + IL1R2 + CX3CR1 + CD3D + CTLA4 | 0.619 | 0.502 | 0.736 |
| D 3/4 | CD177 + IL1R2 + CX3CR1 + CIITA | 0.613 | 0.494 | 0.732 |
| D 3/4 | CD177 + IL1R2 + CX3CR1 + CIITA + CD3D | 0.615 | 0.496 | 0.734 |
| D 3/4 | CD177 + IL1R2 + CX3CR1 + CIITA + CD3D + CTLA4 | 0.612 | 0.492 | 0.732 |
| D 3/4 | CD177 + IL1R2 + CX3CR1 + CIITA + CTLA4 | 0.608 | 0.491 | 0.726 |
| D 3/4 | CD177 + IL1R2 + CX3CR1 + CIITA + TAP2 | 0.619 | 0.503 | 0.735 |
| D 3/4 | CD177 + IL1R2 + CX3CR1 + CIITA + TAP2 + CD3D | 0.625 | 0.503 | 0.746 |
| D 3/4 | CD177 + IL1R2 + CX3CR1 + CIITA + TAP2 + CD3D + CTLA4 | 0.62 | 0.497 | 0.743 |
| D 3/4 | CD177 + IL1R2 + CX3CR1 + CIITA + TAP2 + CTLA4 | 0.612 | 0.496 | 0.728 |
| D 3/4 | CD177 + IL1R2 + CX3CR1 + CTLA4 | 0.614 | 0.499 | 0.73 |
| D 3/4 | CD177 + IL1R2 + CX3CR1 + TAP2 | 0.623 | 0.506 | 0.74 |
| D 3/4 | CD177 + IL1R2 + CX3CR1 + TAP2 + CD3D | 0.627 | 0.507 | 0.746 |
| D 3/4 | CD177 + IL1R2 + CX3CR1 + TAP2 + CD3D + CTLA4 | 0.627 | 0.508 | 0.746 |
| D 3/4 | CD177 + IL1R2 + CX3CR1 + TAP2 + CTLA4 | 0.625 | 0.51 | 0.74 |
| D 3/4 | CD177 + IL1R2 + IFNG | 0.614 | 0.501 | 0.727 |
| D 3/4 | CD177 + IL1R2 + IFNG + CD3D | 0.613 | 0.498 | 0.728 |
| D 3/4 | CD177 + IL1R2 + IFNG + CD3D + CTLA4 | 0.625 | 0.507 | 0.742 |
| D 3/4 | CD177 + IL1R2 + IFNG + CIITA | 0.609 | 0.494 | 0.724 |
| D 3/4 | CD177 + IL1R2 + IFNG + CIITA + CD3D | 0.611 | 0.495 | 0.728 |
| D 3/4 | CD177 + IL1R2 + IFNG + CIITA + CD3D + CTLA4 | 0.613 | 0.494 | 0.732 |
| D 3/4 | CD177 + IL1R2 + IFNG + CIITA + CTLA4 | 0.608 | 0.491 | 0.725 |
| D 3/4 | CD177 + IL1R2 + IFNG + CIITA + TAP2 | 0.619 | 0.51 | 0.729 |
| D 3/4 | CD177 + IL1R2 + IFNG + CIITA + TAP2 + CD3D | 0.627 | 0.512 | 0.741 |
| D 3/4 | CD177 + IL1R2 + IFNG + CIITA + TAP2 + CD3D + CTLA4 | 0.619 | 0.498 | 0.741 |
| D 3/4 | CD177 + IL1R2 + IFNG + CIITA + TAP2 + CTLA4 | 0.609 | 0.495 | 0.723 |
| D 3/4 | CD177 + IL1R2 + IFNG + CTLA4 | 0.615 | 0.5 | 0.731 |
| D 3/4 | CD177 + IL1R2 + IFNG + CX3CR1 | 0.616 | 0.499 | 0.733 |
| D 3/4 | CD177 + IL1R2 + IFNG + CX3CR1 + CD3D | 0.621 | 0.504 | 0.738 |
| D 3/4 | CD177 + IL1R2 + IFNG + CX3CR1 + CD3D + CTLA4 | 0.618 | 0.501 | 0.735 |
| D 3/4 | CD177 + IL1R2 + IFNG + CX3CR1 + CIITA | 0.617 | 0.499 | 0.735 |
| D 3/4 | CD177 + IL1R2 + IFNG + CX3CR1 + CIITA + CD3D | 0.618 | 0.5 | 0.736 |
| D 3/4 | CD177 + IL1R2 + IFNG + CX3CR1 + CIITA + CD3D + CTLA4 | 0.611 | 0.491 | 0.731 |
| D 3/4 | CD177 + IL1R2 + IFNG + CX3CR1 + CIITA + CTLA4 | 0.608 | 0.491 | 0.726 |
| D 3/4 | CD177 + IL1R2 + IFNG + CX3CR1 + CIITA + TAP2 | 0.621 | 0.506 | 0.736 |
| D 3/4 | CD177 + IL1R2 + IFNG + CX3CR1 + CIITA + TAP2 + CD3D | 0.628 | 0.509 | 0.748 |
| D 3/4 | CD177 + IL1R2 + IFNG + CX3CR1 + CIITA + TAP2 + CD3D + CTLA4 | 0.62 | 0.497 | 0.742 |
| D 3/4 | CD177 + IL1R2 + IFNG + CX3CR1 + CIITA + TAP2 + CTLA4 | 0.615 | 0.5 | 0.73 |
| D 3/4 | CD177 + IL1R2 + IFNG + CX3CR1 + CTLA4 | 0.616 | 0.5 | 0.732 |
| D 3/4 | CD177 + IL1R2 + IFNG + CX3CR1 + TAP2 | 0.627 | 0.511 | 0.742 |
| D 3/4 | CD177 + IL1R2 + IFNG + CX3CR1 + TAP2 + CD3D | 0.633 | 0.514 | 0.752 |
| D 3/4 | CD177 + IL1R2 + IFNG + CX3CR1 + TAP2 + CD3D + CTLA4 | 0.627 | 0.507 | 0.747 |
| D 3/4 | CD177 + IL1R2 + IFNG + CX3CR1 + TAP2 + CTLA4 | 0.626 | 0.511 | 0.741 |
| D 3/4 | CD177 + IL1R2 + IFNG + S100A9 | 0.632 | 0.517 | 0.748 |
| D 3/4 | CD177 + IL1R2 + IFNG + S100A9 + CD3D | 0.632 | 0.516 | 0.748 |
| D 3/4 | CD177 + IL1R2 + IFNG + S100A9 + CD3D + CTLA4 | 0.627 | 0.514 | 0.739 |
| D 3/4 | CD177 + IL1R2 + IFNG + S100A9 + CIITA | 0.626 | 0.508 | 0.743 |
| D 3/4 | CD177 + IL1R2 + IFNG + S100A9 + CIITA + CD3D | 0.629 | 0.511 | 0.746 |
| D 3/4 | CD177 + IL1R2 + IFNG + S100A9 + CIITA + CD3D + CTLA4 | 0.623 | 0.507 | 0.739 |
| D 3/4 | CD177 + IL1R2 + IFNG + S100A9 + CIITA + CTLA4 | 0.619 | 0.502 | 0.736 |
| D 3/4 | CD177 + IL1R2 + IFNG + S100A9 + CIITA + TAP2 | 0.634 | 0.522 | 0.747 |
| D 3/4 | CD177 + IL1R2 + IFNG + S100A9 + CIITA + TAP2 + CD3D | 0.642 | 0.529 | 0.755 |
| D 3/4 | CD177 + IL1R2 + IFNG + S100A9 + CIITA + TAP2 + CD3D + CTLA4 | 0.635 | 0.52 | 0.75 |
| D 3/4 | CD177 + IL1R2 + IFNG + S100A9 + CIITA + TAP2 + CTLA4 | 0.631 | 0.518 | 0.744 |
| D 3/4 | CD177 + IL1R2 + IFNG + S100A9 + CTLA4 | 0.623 | 0.51 | 0.736 |
| D 3/4 | CD177 + IL1R2 + IFNG + S100A9 + CX3CR1 | 0.635 | 0.519 | 0.751 |
| D 3/4 | CD177 + IL1R2 + IFNG + S100A9 + CX3CR1 + CD3D | 0.633 | 0.517 | 0.749 |
| D 3/4 | CD177 + IL1R2 + IFNG + S100A9 + CX3CR1 + CD3D + CTLA4 | 0.627 | 0.512 | 0.742 |
| D 3/4 | CD177 + IL1R2 + IFNG + S100A9 + CX3CR1 + CIITA | 0.63 | 0.513 | 0.748 |
| D 3/4 | CD177 + IL1R2 + IFNG + S100A9 + CX3CR1 + CIITA + CD3D | 0.622 | 0.504 | 0.739 |
| D 3/4 | CD177 + IL1R2 + IFNG + S100A9 + CX3CR1 + CIITA + CD3D + CTLA4 | 0.625 | 0.509 | 0.742 |
| D 3/4 | CD177 + IL1R2 + IFNG + S100A9 + CX3CR1 + CIITA + CTLA4 | 0.623 | 0.507 | 0.739 |
| D 3/4 | CD177 + IL1R2 + IFNG + S100A9 + CX3CR1 + CIITA + TAP2 | 0.635 | 0.519 | 0.751 |
| D 3/4 | CD177 + IL1R2 + IFNG + S100A9 + CX3CR1 + CIITA + TAP2 + CD3D | 0.637 | 0.522 | 0.753 |
| D 3/4 | CD177 + IL1R2 + IFNG + S100A9 + CX3CR1 + CIITA + TAP2 + CD3D + CTLA4 | 0.631 | 0.516 | 0.747 |
| D 3/4 | CD177 + IL1R2 + IFNG + S100A9 + CX3CR1 + CIITA + TAP2 + CTLA4 | 0.629 | 0.516 | 0.742 |
| D 3/4 | CD177 + IL1R2 + IFNG + S100A9 + CX3CR1 + CTLA4 | 0.625 | 0.509 | 0.74 |
| D 3/4 | CD177 + IL1R2 + IFNG + S100A9 + CX3CR1 + TAP2 | 0.642 | 0.527 | 0.758 |
| D 3/4 | CD177 + IL1R2 + IFNG + S100A9 + CX3CR1 + TAP2 + CD3D | 0.642 | 0.527 | 0.758 |
| D 3/4 | CD177 + IL1R2 + IFNG + S100A9 + CX3CR1 + TAP2 + CD3D + CTLA4 | 0.637 | 0.521 | 0.752 |
| D 3/4 | CD177 + IL1R2 + IFNG + S100A9 + CX3CR1 + TAP2 + CTLA4 | 0.635 | 0.52 | 0.749 |
| D 3/4 | CD177 + IL1R2 + IFNG + S100A9 + TAP2 | 0.636 | 0.522 | 0.75 |
| D 3/4 | CD177 + IL1R2 + IFNG + S100A9 + TAP2 + CD3D | 0.639 | 0.526 | 0.753 |
| D 3/4 | CD177 + IL1R2 + IFNG + S100A9 + TAP2 + CD3D + CTLA4 | 0.635 | 0.521 | 0.749 |
| D 3/4 | CD177 + IL1R2 + IFNG + S100A9 + TAP2 + CTLA4 | 0.633 | 0.521 | 0.745 |
| D 3/4 | CD177 + IL1R2 + IFNG + TAP2 | 0.625 | 0.515 | 0.735 |
| D 3/4 | CD177 + IL1R2 + IFNG + TAP2 + CD3D | 0.631 | 0.518 | 0.745 |
| D 3/4 | CD177 + IL1R2 + IFNG + TAP2 + CD3D + CTLA4 | 0.624 | 0.508 | 0.739 |
| D 3/4 | CD177 + IL1R2 + IFNG + TAP2 + CTLA4 | 0.616 | 0.503 | 0.729 |
| D 3/4 | CD177 + IL1R2 + S100A9 | 0.636 | 0.523 | 0.749 |
| D 3/4 | CD177 + IL1R2 + S100A9 + CD3D | 0.635 | 0.521 | 0.749 |
| D 3/4 | CD177 + IL1R2 + S100A9 + CD3D + CTLA4 | 0.636 | 0.523 | 0.749 |
| D 3/4 | CD177 + IL1R2 + S100A9 + CIITA | 0.625 | 0.508 | 0.742 |
| D 3/4 | CD177 + IL1R2 + S100A9 + CIITA + CD3D | 0.628 | 0.511 | 0.745 |
| D 3/4 | CD177 + IL1R2 + S100A9 + CIITA + CD3D + CTLA4 | 0.625 | 0.508 | 0.741 |
| D 3/4 | CD177 + IL1R2 + S100A9 + CIITA + CTLA4 | 0.623 | 0.506 | 0.74 |
| D 3/4 | CD177 + IL1R2 + S100A9 + CIITA + TAP2 | 0.634 | 0.522 | 0.746 |
| D 3/4 | CD177 + IL1R2 + S100A9 + CIITA + TAP2 + CD3D | 0.643 | 0.53 | 0.755 |
| D 3/4 | CD177 + IL1R2 + S100A9 + CIITA + TAP2 + CD3D + CTLA4 | 0.637 | 0.522 | 0.752 |
| D 3/4 | CD177 + IL1R2 + S100A9 + CIITA + TAP2 + CTLA4 | 0.628 | 0.515 | 0.74 |
| D 3/4 | CD177 + IL1R2 + S100A9 + CTLA4 | 0.631 | 0.519 | 0.744 |
| D 3/4 | CD177 + IL1R2 + S100A9 + CX3CR1 | 0.633 | 0.516 | 0.749 |
| D 3/4 | CD177 + IL1R2 + S100A9 + CX3CR1 + CD3D | 0.632 | 0.516 | 0.748 |
| D 3/4 | CD177 + IL1R2 + S100A9 + CX3CR1 + CD3D + CTLA4 | 0.63 | 0.515 | 0.744 |
| D 3/4 | CD177 + IL1R2 + S100A9 + CX3CR1 + CIITA | 0.631 | 0.512 | 0.75 |
| D 3/4 | CD177 + IL1R2 + S100A9 + CX3CR1 + CIITA + CD3D | 0.623 | 0.504 | 0.741 |
| D 3/4 | CD177 + IL1R2 + S100A9 + CX3CR1 + CIITA + CD3D + CTLA4 | 0.625 | 0.509 | 0.741 |
| D 3/4 | CD177 + IL1R2 + S100A9 + CX3CR1 + CIITA + CTLA4 | 0.624 | 0.508 | 0.74 |
| D 3/4 | CD177 + IL1R2 + S100A9 + CX3CR1 + CIITA + TAP2 | 0.634 | 0.517 | 0.751 |
| D 3/4 | CD177 + IL1R2 + S100A9 + CX3CR1 + CIITA + TAP2 + CD3D | 0.636 | 0.519 | 0.753 |
| D 3/4 | CD177 + IL1R2 + S100A9 + CX3CR1 + CIITA + TAP2 + CD3D + CTLA4 | 0.635 | 0.519 | 0.75 |
| D 3/4 | CD177 + IL1R2 + S100A9 + CX3CR1 + CIITA + TAP2 + CTLA4 | 0.629 | 0.516 | 0.742 |
| D 3/4 | CD177 + IL1R2 + S100A9 + CX3CR1 + CTLA4 | 0.63 | 0.516 | 0.744 |
| D 3/4 | CD177 + IL1R2 + S100A9 + CX3CR1 + TAP2 | 0.639 | 0.523 | 0.754 |
| D 3/4 | CD177 + IL1R2 + S100A9 + CX3CR1 + TAP2 + CD3D | 0.643 | 0.528 | 0.759 |
| D 3/4 | CD177 + IL1R2 + S100A9 + CX3CR1 + TAP2 + CD3D + CTLA4 | 0.637 | 0.521 | 0.752 |
| D 3/4 | CD177 + IL1R2 + S100A9 + CX3CR1 + TAP2 + CTLA4 | 0.639 | 0.525 | 0.753 |
| D 3/4 | CD177 + IL1R2 + S100A9 + TAP2 | 0.641 | 0.531 | 0.751 |
| D 3/4 | CD177 + IL1R2 + S100A9 + TAP2 + CD3D | 0.646 | 0.536 | 0.757 |
| D 3/4 | CD177 + IL1R2 + S100A9 + TAP2 + CD3D + CTLA4 | 0.645 | 0.533 | 0.758 |
| D 3/4 | CD177 + IL1R2 + S100A9 + TAP2 + CTLA4 | 0.643 | 0.533 | 0.753 |
| D 3/4 | CD177 + IL1R2 + TAP2 | 0.627 | 0.517 | 0.737 |
| D 3/4 | CD177 + IL1R2 + TAP2 + CD3D | 0.633 | 0.521 | 0.746 |
| D 3/4 | CD177 + IL1R2 + TAP2 + CD3D + CTLA4 | 0.627 | 0.513 | 0.742 |
| D 3/4 | CD177 + IL1R2 + TAP2 + CTLA4 | 0.628 | 0.516 | 0.74 |
| D 3/4 | CD177 + S100A9 + CD3D | 0.643 | 0.536 | 0.75 |
| D 3/4 | CD177 + S100A9 + CD3D + CTLA4 | 0.639 | 0.533 | 0.746 |
| D 3/4 | CD177 + S100A9 + CIITA | 0.637 | 0.527 | 0.747 |
| D 3/4 | CD177 + S100A9 + CIITA + CD3D | 0.637 | 0.527 | 0.747 |
| D 3/4 | CD177 + S100A9 + CIITA + CD3D + CTLA4 | 0.641 | 0.532 | 0.75 |
| D 3/4 | CD177 + S100A9 + CIITA + CTLA4 | 0.634 | 0.525 | 0.744 |
| D 3/4 | CD177 + S100A9 + CIITA + TAP2 | 0.637 | 0.527 | 0.747 |
| D 3/4 | CD177 + S100A9 + CIITA + TAP2 + CD3D | 0.638 | 0.528 | 0.747 |
| D 3/4 | CD177 + S100A9 + CIITA + TAP2 + CD3D + CTLA4 | 0.641 | 0.532 | 0.75 |
| D 3/4 | CD177 + S100A9 + CIITA + TAP2 + CTLA4 | 0.634 | 0.525 | 0.744 |
| D 3/4 | CD177 + S100A9 + CTLA4 | 0.64 | 0.534 | 0.746 |
| D 3/4 | CD177 + S100A9 + CX3CR1 | 0.642 | 0.53 | 0.754 |
| D 3/4 | CD177 + S100A9 + CX3CR1 + CD3D | 0.641 | 0.529 | 0.753 |
| D 3/4 | CD177 + S100A9 + CX3CR1 + CD3D + CTLA4 | 0.642 | 0.532 | 0.752 |
| D 3/4 | CD177 + S100A9 + CX3CR1 + CIITA | 0.641 | 0.527 | 0.755 |
| D 3/4 | CD177 + S100A9 + CX3CR1 + CIITA + CD3D | 0.64 | 0.528 | 0.753 |
| D 3/4 | CD177 + S100A9 + CX3CR1 + CIITA + CD3D + CTLA4 | 0.638 | 0.527 | 0.749 |
| D 3/4 | CD177 + S100A9 + CX3CR1 + CIITA + CTLA4 | 0.638 | 0.527 | 0.749 |
| D 3/4 | CD177 + S100A9 + CX3CR1 + CIITA + TAP2 | 0.647 | 0.534 | 0.761 |
| D 3/4 | CD177 + S100A9 + CX3CR1 + CIITA + TAP2 + CD3D | 0.641 | 0.529 | 0.753 |
| D 3/4 | CD177 + S100A9 + CX3CR1 + CIITA + TAP2 + CD3D + CTLA4 | 0.64 | 0.529 | 0.751 |
| D 3/4 | CD177 + S100A9 + CX3CR1 + CIITA + TAP2 + CTLA4 | 0.639 | 0.528 | 0.75 |
| D 3/4 | CD177 + S100A9 + CX3CR1 + CTLA4 | 0.641 | 0.53 | 0.751 |
| D 3/4 | CD177 + S100A9 + CX3CR1 + TAP2 | 0.644 | 0.531 | 0.756 |
| D 3/4 | CD177 + S100A9 + CX3CR1 + TAP2 + CD3D | 0.643 | 0.531 | 0.755 |
| D 3/4 | CD177 + S100A9 + CX3CR1 + TAP2 + CD3D + CTLA4 | 0.64 | 0.529 | 0.751 |
| D 3/4 | CD177 + S100A9 + CX3CR1 + TAP2 + CTLA4 | 0.641 | 0.529 | 0.752 |
| D 3/4 | CD177 + S100A9 + TAP2 | 0.642 | 0.535 | 0.75 |
| D 3/4 | CD177 + S100A9 + TAP2 + CD3D | 0.642 | 0.534 | 0.749 |
| D 3/4 | CD177 + S100A9 + TAP2 + CD3D + CTLA4 | 0.638 | 0.531 | 0.745 |
| D 3/4 | CD177 + S100A9 + TAP2 + CTLA4 | 0.64 | 0.532 | 0.747 |
| D 3/4 | CD177 + TAP2 + CD3D | 0.61 | 0.5 | 0.721 |
| D 3/4 | CD177 + TAP2 + CD3D + CTLA4 | 0.61 | 0.498 | 0.723 |
| D 3/4 | CD177 + TAP2 + CTLA4 | 0.606 | 0.497 | 0.715 |
| D 5/7 | CD177 + GSN | 0.677 | 0.556 | 0.798 |
| D 5/7 | CD177 + IL10 | 0.678 | 0.558 | 0.797 |
| D 5/7 | CD177 + MDC1 | 0.68 | 0.558 | 0.802 |
| D 5/7 | CD177 + HAVCR2 | 0.68 | 0.547 | 0.812 |
| D 5/7 | CD177 + IL18 | 0.681 | 0.558 | 0.803 |
| D 5/7 | CD177 + RORC | 0.682 | 0.554 | 0.809 |
| D 5/7 | CD177 + CXCL10 | 0.683 | 0.557 | 0.809 |
| D 5/7 | CD177 + CTLA4 | 0.687 | 0.556 | 0.818 |
| D 5/7 | CD177 + GNLY | 0.688 | 0.575 | 0.8 |
| D 5/7 | CD177 + CIITA | 0.692 | 0.584 | 0.801 |
| D 5/7 | CD177 + IL1RN | 0.695 | 0.578 | 0.811 |
| D 5/7 | CD177 + ADGRE3 | 0.697 | 0.568 | 0.826 |
| D 5/7 | CD177 + ARL14EP | 0.698 | 0.6 | 0.797 |
| D 5/7 | CD177 + OAS2 | 0.699 | 0.568 | 0.83 |
| D 5/7 | CD177 + TDRD9 | 0.702 | 0.588 | 0.815 |
| D 5/7 | CD177 + FLT1 | 0.704 | 0.603 | 0.805 |
| D 5/7 | CD177 + CD3D | 0.706 | 0.588 | 0.824 |
| D 5/7 | CD177 + GATA3 | 0.708 | 0.585 | 0.831 |
| D 5/7 | CD177 + ZAP70 | 0.713 | 0.607 | 0.819 |
| D 5/7 | CD177 + IL6 | 0.716 | 0.612 | 0.82 |
| D 5/7 | CD177 + S100A9 | 0.728 | 0.628 | 0.828 |
| D 5/7 | CD177 + CX3CR1 | 0.73 | 0.613 | 0.848 |
| D 5/7 | CD177 + TAP2 | 0.759 | 0.662 | 0.856 |
| D 5/7 | CD177 + C3AR1 | 0.739 | 0.634 | 0.844 |
| D 5/7 | CD177 + C3AR1 + CD3D | 0.725 | 0.615 | 0.835 |
| D 5/7 | CD177 + C3AR1 + CD3D + CTLA4 | 0.726 | 0.612 | 0.84 |
| D 5/7 | CD177 + C3AR1 + CD74 | 0.759 | 0.646 | 0.872 |
| D 5/7 | CD177 + C3AR1 + CD74 + CD3D | 0.758 | 0.646 | 0.869 |
| D 5/7 | CD177 + C3AR1 + CD74 + CD3D + CTLA4 | 0.752 | 0.641 | 0.863 |
| D 5/7 | CD177 + C3AR1 + CD74 + CIITA | 0.745 | 0.625 | 0.866 |
| D 5/7 | CD177 + C3AR1 + CD74 + CIITA + CD3D | 0.75 | 0.636 | 0.865 |
| D 5/7 | CD177 + C3AR1 + CD74 + CIITA + CD3D + CTLA4 | 0.75 | 0.634 | 0.866 |
| D 5/7 | CD177 + C3AR1 + CD74 + CIITA + CTLA4 | 0.742 | 0.619 | 0.865 |
| D 5/7 | CD177 + C3AR1 + CD74 + CIITA + TAP2 | 0.768 | 0.661 | 0.875 |
| D 5/7 | CD177 + C3AR1 + CD74 + CIITA + TAP2 + CD3D | 0.763 | 0.658 | 0.868 |
| D 5/7 | CD177 + C3AR1 + CD74 + CIITA + TAP2 + CD3D + CTLA4 | 0.767 | 0.66 | 0.873 |
| D 5/7 | CD177 + C3AR1 + CD74 + CIITA + TAP2 + CTLA4 | 0.769 | 0.664 | 0.875 |
| D 5/7 | CD177 + C3AR1 + CD74 + CTLA4 | 0.753 | 0.636 | 0.87 |
| D 5/7 | CD177 + C3AR1 + CD74 + CX3CR1 | 0.767 | 0.658 | 0.876 |
| D 5/7 | CD177 + C3AR1 + CD74 + CX3CR1 + CD3D | 0.761 | 0.649 | 0.873 |
| D 5/7 | CD177 + C3AR1 + CD74 + CX3CR1 + CD3D + CTLA4 | 0.757 | 0.643 | 0.872 |
| D 5/7 | CD177 + C3AR1 + CD74 + CX3CR1 + CIITA | 0.754 | 0.636 | 0.872 |
| D 5/7 | CD177 + C3AR1 + CD74 + CX3CR1 + CIITA + CD3D | 0.755 | 0.638 | 0.872 |
| D 5/7 | CD177 + C3AR1 + CD74 + CX3CR1 + CIITA + CD3D + CTLA4 | 0.754 | 0.634 | 0.873 |
| D 5/7 | CD177 + C3AR1 + CD74 + CX3CR1 + CIITA + CTLA4 | 0.75 | 0.627 | 0.874 |
| D 5/7 | CD177 + C3AR1 + CD74 + CX3CR1 + CIITA + TAP2 | 0.769 | 0.661 | 0.876 |
| D 5/7 | CD177 + C3AR1 + CD74 + CX3CR1 + CIITA + TAP2 + CD3D | 0.763 | 0.658 | 0.869 |
| D 5/7 | CD177 + C3AR1 + CD74 + CX3CR1 + CIITA + TAP2 + CD3D + CTLA4 | 0.765 | 0.659 | 0.872 |
| D 5/7 | CD177 + C3AR1 + CD74 + CX3CR1 + CIITA + TAP2 + CTLA4 | 0.767 | 0.658 | 0.876 |
| D 5/7 | CD177 + C3AR1 + CD74 + CX3CR1 + CTLA4 | 0.764 | 0.649 | 0.879 |
| D 5/7 | CD177 + C3AR1 + CD74 + CX3CR1 + TAP2 | 0.778 | 0.678 | 0.879 |
| D 5/7 | CD177 + C3AR1 + CD74 + CX3CR1 + TAP2 + CD3D | 0.773 | 0.673 | 0.874 |
| D 5/7 | CD177 + C3AR1 + CD74 + CX3CR1 + TAP2 + CD3D + CTLA4 | 0.769 | 0.667 | 0.872 |
| D 5/7 | CD177 + C3AR1 + CD74 + CX3CR1 + TAP2 + CTLA4 | 0.771 | 0.668 | 0.874 |
| D 5/7 | CD177 + C3AR1 + CD74 + IFNG | 0.755 | 0.634 | 0.877 |
| D 5/7 | CD177 + C3AR1 + CD74 + IFNG + CD3D | 0.754 | 0.625 | 0.882 |
| D 5/7 | CD177 + C3AR1 + CD74 + IFNG + CD3D + CTLA4 | 0.75 | 0.62 | 0.881 |
| D 5/7 | CD177 + C3AR1 + CD74 + IFNG + CIITA | 0.756 | 0.633 | 0.879 |
| D 5/7 | CD177 + C3AR1 + CD74 + IFNG + CIITA + CD3D | 0.754 | 0.628 | 0.881 |
| D 5/7 | CD177 + C3AR1 + CD74 + IFNG + CIITA + CD3D + CTLA4 | 0.751 | 0.622 | 0.879 |
| D 5/7 | CD177 + C3AR1 + CD74 + IFNG + CIITA + CTLA4 | 0.747 | 0.618 | 0.876 |
| D 5/7 | CD177 + C3AR1 + CD74 + IFNG + CIITA + TAP2 | 0.778 | 0.67 | 0.886 |
| D 5/7 | CD177 + C3AR1 + CD74 + IFNG + CIITA + TAP2 + CD3D | 0.769 | 0.653 | 0.885 |
| D 5/7 | CD177 + C3AR1 + CD74 + IFNG + CIITA + TAP2 + CD3D + CTLA4 | 0.77 | 0.655 | 0.885 |
| D 5/7 | CD177 + C3AR1 + CD74 + IFNG + CIITA + TAP2 + CTLA4 | 0.776 | 0.665 | 0.887 |
| D 5/7 | CD177 + C3AR1 + CD74 + IFNG + CTLA4 | 0.743 | 0.614 | 0.872 |
| D 5/7 | CD177 + C3AR1 + CD74 + IFNG + CX3CR1 | 0.754 | 0.628 | 0.881 |
| D 5/7 | CD177 + C3AR1 + CD74 + IFNG + CX3CR1 + CD3D | 0.759 | 0.63 | 0.888 |
| D 5/7 | CD177 + C3AR1 + CD74 + IFNG + CX3CR1 + CD3D + CTLA4 | 0.751 | 0.616 | 0.885 |
| D 5/7 | CD177 + C3AR1 + CD74 + IFNG + CX3CR1 + CIITA | 0.754 | 0.629 | 0.88 |
| D 5/7 | CD177 + C3AR1 + CD74 + IFNG + CX3CR1 + CIITA + CD3D | 0.758 | 0.629 | 0.886 |
| D 5/7 | CD177 + C3AR1 + CD74 + IFNG + CX3CR1 + CIITA + CD3D + CTLA4 | 0.754 | 0.621 | 0.886 |
| D 5/7 | CD177 + C3AR1 + CD74 + IFNG + CX3CR1 + CIITA + CTLA4 | 0.752 | 0.621 | 0.882 |
| D 5/7 | CD177 + C3AR1 + CD74 + IFNG + CX3CR1 + CIITA + TAP2 | 0.78 | 0.671 | 0.889 |
| D 5/7 | CD177 + C3AR1 + CD74 + IFNG + CX3CR1 + CIITA + TAP2 + CD3D | 0.774 | 0.659 | 0.889 |
| D 5/7 | CD177 + C3AR1 + CD74 + IFNG + CX3CR1 + CIITA + TAP2 + CD3D + CTLA4 | 0.772 | 0.656 | 0.888 |
| D 5/7 | CD177 + C3AR1 + CD74 + IFNG + CX3CR1 + CIITA + TAP2 + CTLA4 | 0.778 | 0.665 | 0.891 |
| D 5/7 | CD177 + C3AR1 + CD74 + IFNG + CX3CR1 + CTLA4 | 0.751 | 0.62 | 0.883 |
| D 5/7 | CD177 + C3AR1 + CD74 + IFNG + CX3CR1 + TAP2 | 0.778 | 0.667 | 0.888 |
| D 5/7 | CD177 + C3AR1 + CD74 + IFNG + CX3CR1 + TAP2 + CD3D | 0.772 | 0.656 | 0.887 |
| D 5/7 | CD177 + C3AR1 + CD74 + IFNG + CX3CR1 + TAP2 + CD3D + CTLA4 | 0.77 | 0.656 | 0.885 |
| D 5/7 | CD177 + C3AR1 + CD74 + IFNG + CX3CR1 + TAP2 + CTLA4 | 0.778 | 0.664 | 0.891 |
| D 5/7 | CD177 + C3AR1 + CD74 + IFNG + S100A9 | 0.773 | 0.668 | 0.879 |
| D 5/7 | CD177 + C3AR1 + CD74 + IFNG + S100A9 + CD3D | 0.789 | 0.683 | 0.894 |
| D 5/7 | CD177 + C3AR1 + CD74 + IFNG + S100A9 + CD3D + CTLA4 | 0.791 | 0.687 | 0.896 |
| D 5/7 | CD177 + C3AR1 + CD74 + IFNG + S100A9 + CIITA | 0.767 | 0.657 | 0.877 |
| D 5/7 | CD177 + C3AR1 + CD74 + IFNG + S100A9 + CIITA + CD3D | 0.79 | 0.682 | 0.898 |
| D 5/7 | CD177 + C3AR1 + CD74 + IFNG + S100A9 + CIITA + CD3D + CTLA4 | 0.788 | 0.68 | 0.896 |
| D 5/7 | CD177 + C3AR1 + CD74 + IFNG + S100A9 + CIITA + CTLA4 | 0.763 | 0.644 | 0.882 |
| D 5/7 | CD177 + C3AR1 + CD74 + IFNG + S100A9 + CIITA + TAP2 | 0.801 | 0.707 | 0.894 |
| D 5/7 | CD177 + C3AR1 + CD74 + IFNG + S100A9 + CIITA + TAP2 + CD3D | 0.8 | 0.704 | 0.896 |
| D 5/7 | CD177 + C3AR1 + CD74 + IFNG + S100A9 + CIITA + TAP2 + CD3D + CTLA4 | 0.806 | 0.711 | 0.901 |
| D 5/7 | CD177 + C3AR1 + CD74 + IFNG + S100A9 + CIITA + TAP2 + CTLA4 | 0.797 | 0.701 | 0.892 |
| D 5/7 | CD177 + C3AR1 + CD74 + IFNG + S100A9 + CTLA4 | 0.777 | 0.668 | 0.885 |
| D 5/7 | CD177 + C3AR1 + CD74 + IFNG + S100A9 + CX3CR1 | 0.807 | 0.709 | 0.904 |
| D 5/7 | CD177 + C3AR1 + CD74 + IFNG + S100A9 + CX3CR1 + CD3D | 0.802 | 0.695 | 0.909 |
| D 5/7 | CD177 + C3AR1 + CD74 + IFNG + S100A9 + CX3CR1 + CD3D + CTLA4 | 0.802 | 0.695 | 0.909 |
| D 5/7 | CD177 + C3AR1 + CD74 + IFNG + S100A9 + CX3CR1 + CIITA | 0.802 | 0.7 | 0.903 |
| D 5/7 | CD177 + C3AR1 + CD74 + IFNG + S100A9 + CX3CR1 + CIITA + CD3D | 0.8 | 0.691 | 0.909 |
| D 5/7 | CD177 + C3AR1 + CD74 + IFNG + S100A9 + CX3CR1 + CIITA + CD3D + CTLA4 | 0.8 | 0.691 | 0.909 |
| D 5/7 | CD177 + C3AR1 + CD74 + IFNG + S100A9 + CX3CR1 + CIITA + CTLA4 | 0.795 | 0.681 | 0.908 |
| D 5/7 | CD177 + C3AR1 + CD74 + IFNG + S100A9 + CX3CR1 + CIITA + TAP2 | 0.814 | 0.719 | 0.909 |
| D 5/7 | CD177 + C3AR1 + CD74 + IFNG + S100A9 + CX3CR1 + CIITA + TAP2 + CD3D | 0.808 | 0.709 | 0.906 |
| D 5/7 | CD177 + C3AR1 + CD74 + IFNG + S100A9 + CX3CR1 + CIITA + TAP2 + CD3D + CTLA4 | 0.81 | 0.713 | 0.907 |
| D 5/7 | CD177 + C3AR1 + CD74 + IFNG + S100A9 + CX3CR1 + CIITA + TAP2 + CTLA4 | 0.813 | 0.715 | 0.912 |
| D 5/7 | CD177 + C3AR1 + CD74 + IFNG + S100A9 + CX3CR1 + CTLA4 | 0.799 | 0.693 | 0.906 |
| D 5/7 | CD177 + C3AR1 + CD74 + IFNG + S100A9 + CX3CR1 + TAP2 | 0.82 | 0.729 | 0.912 |
| D 5/7 | CD177 + C3AR1 + CD74 + IFNG + S100A9 + CX3CR1 + TAP2 + CD3D | 0.812 | 0.715 | 0.909 |
| D 5/7 | CD177 + C3AR1 + CD74 + IFNG + S100A9 + CX3CR1 + TAP2 + CD3D + CTLA4 | 0.81 | 0.713 | 0.906 |
| D 5/7 | CD177 + C3AR1 + CD74 + IFNG + S100A9 + CX3CR1 + TAP2 + CTLA4 | 0.819 | 0.725 | 0.913 |
| D 5/7 | CD177 + C3AR1 + CD74 + IFNG + S100A9 + TAP2 | 0.801 | 0.709 | 0.893 |
| D 5/7 | CD177 + C3AR1 + CD74 + IFNG + S100A9 + TAP2 + CD3D | 0.799 | 0.703 | 0.896 |
| D 5/7 | CD177 + C3AR1 + CD74 + IFNG + S100A9 + TAP2 + CD3D + CTLA4 | 0.809 | 0.715 | 0.903 |
| D 5/7 | CD177 + C3AR1 + CD74 + IFNG + S100A9 + TAP2 + CTLA4 | 0.801 | 0.709 | 0.894 |
| D 5/7 | CD177 + C3AR1 + CD74 + IFNG + TAP2 | 0.778 | 0.67 | 0.886 |
| D 5/7 | CD177 + C3AR1 + CD74 + IFNG + TAP2 + CD3D | 0.769 | 0.654 | 0.884 |
| D 5/7 | CD177 + C3AR1 + CD74 + IFNG + TAP2 + CD3D + CTLA4 | 0.769 | 0.654 | 0.884 |
| D 5/7 | CD177 + C3AR1 + CD74 + IFNG + TAP2 + CTLA4 | 0.775 | 0.664 | 0.887 |
| D 5/7 | CD177 + C3AR1 + CD74 + S100A9 | 0.768 | 0.665 | 0.87 |
| D 5/7 | CD177 + C3AR1 + CD74 + S100A9 + CD3D | 0.774 | 0.68 | 0.868 |
| D 5/7 | CD177 + C3AR1 + CD74 + S100A9 + CD3D + CTLA4 | 0.771 | 0.677 | 0.865 |
| D 5/7 | CD177 + C3AR1 + CD74 + S100A9 + CIITA | 0.762 | 0.654 | 0.87 |
| D 5/7 | CD177 + C3AR1 + CD74 + S100A9 + CIITA + CD3D | 0.772 | 0.673 | 0.871 |
| D 5/7 | CD177 + C3AR1 + CD74 + S100A9 + CIITA + CD3D + CTLA4 | 0.772 | 0.669 | 0.874 |
| D 5/7 | CD177 + C3AR1 + CD74 + S100A9 + CIITA + CTLA4 | 0.753 | 0.637 | 0.869 |
| D 5/7 | CD177 + C3AR1 + CD74 + S100A9 + CIITA + TAP2 | 0.785 | 0.688 | 0.882 |
| D 5/7 | CD177 + C3AR1 + CD74 + S100A9 + CIITA + TAP2 + CD3D | 0.783 | 0.689 | 0.876 |
| D 5/7 | CD177 + C3AR1 + CD74 + S100A9 + CIITA + TAP2 + CD3D + CTLA4 | 0.785 | 0.692 | 0.878 |
| D 5/7 | CD177 + C3AR1 + CD74 + S100A9 + CIITA + TAP2 + CTLA4 | 0.782 | 0.683 | 0.881 |
| D 5/7 | CD177 + C3AR1 + CD74 + S100A9 + CTLA4 | 0.763 | 0.659 | 0.868 |
| D 5/7 | CD177 + C3AR1 + CD74 + S100A9 + CX3CR1 | 0.788 | 0.698 | 0.878 |
| D 5/7 | CD177 + C3AR1 + CD74 + S100A9 + CX3CR1 + CD3D | 0.784 | 0.688 | 0.881 |
| D 5/7 | CD177 + C3AR1 + CD74 + S100A9 + CX3CR1 + CD3D + CTLA4 | 0.782 | 0.684 | 0.88 |
| D 5/7 | CD177 + C3AR1 + CD74 + S100A9 + CX3CR1 + CIITA | 0.782 | 0.68 | 0.884 |
| D 5/7 | CD177 + C3AR1 + CD74 + S100A9 + CX3CR1 + CIITA + CD3D | 0.783 | 0.682 | 0.883 |
| D 5/7 | CD177 + C3AR1 + CD74 + S100A9 + CX3CR1 + CIITA + CD3D + CTLA4 | 0.775 | 0.671 | 0.879 |
| D 5/7 | CD177 + C3AR1 + CD74 + S100A9 + CX3CR1 + CIITA + CTLA4 | 0.771 | 0.657 | 0.885 |
| D 5/7 | CD177 + C3AR1 + CD74 + S100A9 + CX3CR1 + CIITA + TAP2 | 0.793 | 0.698 | 0.888 |
| D 5/7 | CD177 + C3AR1 + CD74 + S100A9 + CX3CR1 + CIITA + TAP2 + CD3D | 0.79 | 0.696 | 0.884 |
| D 5/7 | CD177 + C3AR1 + CD74 + S100A9 + CX3CR1 + CIITA + TAP2 + CD3D + CTLA4 | 0.791 | 0.698 | 0.884 |
| D 5/7 | CD177 + C3AR1 + CD74 + S100A9 + CX3CR1 + CIITA + TAP2 + CTLA4 | 0.791 | 0.694 | 0.889 |
| D 5/7 | CD177 + C3AR1 + CD74 + S100A9 + CX3CR1 + CTLA4 | 0.781 | 0.684 | 0.879 |
| D 5/7 | CD177 + C3AR1 + CD74 + S100A9 + CX3CR1 + TAP2 | 0.792 | 0.704 | 0.879 |
| D 5/7 | CD177 + C3AR1 + CD74 + S100A9 + CX3CR1 + TAP2 + CD3D | 0.789 | 0.7 | 0.877 |
| D 5/7 | CD177 + C3AR1 + CD74 + S100A9 + CX3CR1 + TAP2 + CD3D + CTLA4 | 0.792 | 0.706 | 0.879 |
| D 5/7 | CD177 + C3AR1 + CD74 + S100A9 + CX3CR1 + TAP2 + CTLA4 | 0.791 | 0.7 | 0.881 |
| D 5/7 | CD177 + C3AR1 + CD74 + S100A9 + TAP2 | 0.792 | 0.703 | 0.882 |
| D 5/7 | CD177 + C3AR1 + CD74 + S100A9 + TAP2 + CD3D | 0.785 | 0.697 | 0.872 |
| D 5/7 | CD177 + C3AR1 + CD74 + S100A9 + TAP2 + CD3D + CTLA4 | 0.785 | 0.698 | 0.872 |
| D 5/7 | CD177 + C3AR1 + CD74 + S100A9 + TAP2 + CTLA4 | 0.789 | 0.696 | 0.881 |
| D 5/7 | CD177 + C3AR1 + CD74 + TAP2 | 0.773 | 0.671 | 0.875 |
| D 5/7 | CD177 + C3AR1 + CD74 + TAP2 + CD3D | 0.773 | 0.672 | 0.873 |
| D 5/7 | CD177 + C3AR1 + CD74 + TAP2 + CD3D + CTLA4 | 0.77 | 0.668 | 0.871 |
| D 5/7 | CD177 + C3AR1 + CD74 + TAP2 + CTLA4 | 0.769 | 0.665 | 0.873 |
| D 5/7 | CD177 + C3AR1 + CIITA | 0.756 | 0.662 | 0.851 |
| D 5/7 | CD177 + C3AR1 + CIITA + CD3D | 0.745 | 0.648 | 0.842 |
| D 5/7 | CD177 + C3AR1 + CIITA + CD3D + CTLA4 | 0.739 | 0.636 | 0.842 |
| D 5/7 | CD177 + C3AR1 + CIITA + CTLA4 | 0.739 | 0.634 | 0.844 |
| D 5/7 | CD177 + C3AR1 + CIITA + TAP2 | 0.775 | 0.683 | 0.867 |
| D 5/7 | CD177 + C3AR1 + CIITA + TAP2 + CD3D | 0.775 | 0.686 | 0.864 |
| D 5/7 | CD177 + C3AR1 + CIITA + TAP2 + CD3D + CTLA4 | 0.772 | 0.682 | 0.862 |
| D 5/7 | CD177 + C3AR1 + CIITA + TAP2 + CTLA4 | 0.773 | 0.679 | 0.866 |
| D 5/7 | CD177 + C3AR1 + CTLA4 | 0.726 | 0.607 | 0.844 |
| D 5/7 | CD177 + C3AR1 + CX3CR1 | 0.76 | 0.659 | 0.862 |
| D 5/7 | CD177 + C3AR1 + CX3CR1 + CD3D | 0.748 | 0.638 | 0.859 |
| D 5/7 | CD177 + C3AR1 + CX3CR1 + CD3D + CTLA4 | 0.743 | 0.624 | 0.861 |
| D 5/7 | CD177 + C3AR1 + CX3CR1 + CIITA | 0.767 | 0.674 | 0.859 |
| D 5/7 | CD177 + C3AR1 + CX3CR1 + CIITA + CD3D | 0.763 | 0.666 | 0.86 |
| D 5/7 | CD177 + C3AR1 + CX3CR1 + CIITA + CD3D + CTLA4 | 0.754 | 0.647 | 0.86 |
| D 5/7 | CD177 + C3AR1 + CX3CR1 + CIITA + CTLA4 | 0.756 | 0.65 | 0.862 |
| D 5/7 | CD177 + C3AR1 + CX3CR1 + CIITA + TAP2 | 0.778 | 0.688 | 0.868 |
| D 5/7 | CD177 + C3AR1 + CX3CR1 + CIITA + TAP2 + CD3D | 0.777 | 0.689 | 0.866 |
| D 5/7 | CD177 + C3AR1 + CX3CR1 + CIITA + TAP2 + CD3D + CTLA4 | 0.774 | 0.683 | 0.864 |
| D 5/7 | CD177 + C3AR1 + CX3CR1 + CIITA + TAP2 + CTLA4 | 0.774 | 0.682 | 0.865 |
| D 5/7 | CD177 + C3AR1 + CX3CR1 + CTLA4 | 0.746 | 0.629 | 0.863 |
| D 5/7 | CD177 + C3AR1 + CX3CR1 + TAP2 | 0.772 | 0.68 | 0.863 |
| D 5/7 | CD177 + C3AR1 + CX3CR1 + TAP2 + CD3D | 0.772 | 0.68 | 0.863 |
| D 5/7 | CD177 + C3AR1 + CX3CR1 + TAP2 + CD3D + CTLA4 | 0.769 | 0.676 | 0.862 |
| D 5/7 | CD177 + C3AR1 + CX3CR1 + TAP2 + CTLA4 | 0.768 | 0.674 | 0.863 |
| D 5/7 | CD177 + C3AR1 + IFNG | 0.704 | 0.583 | 0.825 |
| D 5/7 | CD177 + C3AR1 + IFNG + CD3D | 0.709 | 0.582 | 0.835 |
| D 5/7 | CD177 + C3AR1 + IFNG + CD3D + CTLA4 | 0.703 | 0.576 | 0.831 |
| D 5/7 | CD177 + C3AR1 + IFNG + CIITA | 0.739 | 0.635 | 0.844 |
| D 5/7 | CD177 + C3AR1 + IFNG + CIITA + CD3D | 0.751 | 0.637 | 0.865 |
| D 5/7 | CD177 + C3AR1 + IFNG + CIITA + CD3D + CTLA4 | 0.741 | 0.627 | 0.855 |
| D 5/7 | CD177 + C3AR1 + IFNG + CIITA + CTLA4 | 0.727 | 0.61 | 0.844 |
| D 5/7 | CD177 + C3AR1 + IFNG + CIITA + TAP2 | 0.773 | 0.672 | 0.874 |
| D 5/7 | CD177 + C3AR1 + IFNG + CIITA + TAP2 + CD3D | 0.769 | 0.667 | 0.872 |
| D 5/7 | CD177 + C3AR1 + IFNG + CIITA + TAP2 + CD3D + CTLA4 | 0.77 | 0.668 | 0.872 |
| D 5/7 | CD177 + C3AR1 + IFNG + CIITA + TAP2 + CTLA4 | 0.775 | 0.675 | 0.875 |
| D 5/7 | CD177 + C3AR1 + IFNG + CTLA4 | 0.695 | 0.567 | 0.822 |
| D 5/7 | CD177 + C3AR1 + IFNG + CX3CR1 | 0.734 | 0.614 | 0.854 |
| D 5/7 | CD177 + C3AR1 + IFNG + CX3CR1 + CD3D | 0.73 | 0.602 | 0.858 |
| D 5/7 | CD177 + C3AR1 + IFNG + CX3CR1 + CD3D + CTLA4 | 0.719 | 0.586 | 0.853 |
| D 5/7 | CD177 + C3AR1 + IFNG + CX3CR1 + CIITA | 0.766 | 0.664 | 0.867 |
| D 5/7 | CD177 + C3AR1 + IFNG + CX3CR1 + CIITA + CD3D | 0.759 | 0.644 | 0.874 |
| D 5/7 | CD177 + C3AR1 + IFNG + CX3CR1 + CIITA + CD3D + CTLA4 | 0.758 | 0.642 | 0.875 |
| D 5/7 | CD177 + C3AR1 + IFNG + CX3CR1 + CIITA + CTLA4 | 0.755 | 0.641 | 0.868 |
| D 5/7 | CD177 + C3AR1 + IFNG + CX3CR1 + CIITA + TAP2 | 0.784 | 0.686 | 0.881 |
| D 5/7 | CD177 + C3AR1 + IFNG + CX3CR1 + CIITA + TAP2 + CD3D | 0.776 | 0.674 | 0.878 |
| D 5/7 | CD177 + C3AR1 + IFNG + CX3CR1 + CIITA + TAP2 + CD3D + CTLA4 | 0.776 | 0.674 | 0.878 |
| D 5/7 | CD177 + C3AR1 + IFNG + CX3CR1 + CIITA + TAP2 + CTLA4 | 0.78 | 0.678 | 0.881 |
| D 5/7 | CD177 + C3AR1 + IFNG + CX3CR1 + CTLA4 | 0.721 | 0.59 | 0.853 |
| D 5/7 | CD177 + C3AR1 + IFNG + CX3CR1 + TAP2 | 0.76 | 0.653 | 0.866 |
| D 5/7 | CD177 + C3AR1 + IFNG + CX3CR1 + TAP2 + CD3D | 0.757 | 0.649 | 0.864 |
| D 5/7 | CD177 + C3AR1 + IFNG + CX3CR1 + TAP2 + CD3D + CTLA4 | 0.756 | 0.646 | 0.866 |
| D 5/7 | CD177 + C3AR1 + IFNG + CX3CR1 + TAP2 + CTLA4 | 0.757 | 0.647 | 0.868 |
| D 5/7 | CD177 + C3AR1 + IFNG + S100A9 | 0.741 | 0.634 | 0.848 |
| D 5/7 | CD177 + C3AR1 + IFNG + S100A9 + CD3D | 0.763 | 0.645 | 0.881 |
| D 5/7 | CD177 + C3AR1 + IFNG + S100A9 + CD3D + CTLA4 | 0.762 | 0.644 | 0.88 |
| D 5/7 | CD177 + C3AR1 + IFNG + S100A9 + CIITA | 0.758 | 0.663 | 0.853 |
| D 5/7 | CD177 + C3AR1 + IFNG + S100A9 + CIITA + CD3D | 0.77 | 0.658 | 0.882 |
| D 5/7 | CD177 + C3AR1 + IFNG + S100A9 + CIITA + CD3D + CTLA4 | 0.767 | 0.654 | 0.88 |
| D 5/7 | CD177 + C3AR1 + IFNG + S100A9 + CIITA + CTLA4 | 0.744 | 0.632 | 0.857 |
| D 5/7 | CD177 + C3AR1 + IFNG + S100A9 + CIITA + TAP2 | 0.796 | 0.705 | 0.886 |
| D 5/7 | CD177 + C3AR1 + IFNG + S100A9 + CIITA + TAP2 + CD3D | 0.795 | 0.696 | 0.893 |
| D 5/7 | CD177 + C3AR1 + IFNG + S100A9 + CIITA + TAP2 + CD3D + CTLA4 | 0.795 | 0.698 | 0.891 |
| D 5/7 | CD177 + C3AR1 + IFNG + S100A9 + CIITA + TAP2 + CTLA4 | 0.795 | 0.702 | 0.887 |
| D 5/7 | CD177 + C3AR1 + IFNG + S100A9 + CTLA4 | 0.738 | 0.619 | 0.857 |
| D 5/7 | CD177 + C3AR1 + IFNG + S100A9 + CX3CR1 | 0.791 | 0.683 | 0.899 |
| D 5/7 | CD177 + C3AR1 + IFNG + S100A9 + CX3CR1 + CD3D | 0.781 | 0.658 | 0.904 |
| D 5/7 | CD177 + C3AR1 + IFNG + S100A9 + CX3CR1 + CD3D + CTLA4 | 0.783 | 0.659 | 0.907 |
| D 5/7 | CD177 + C3AR1 + IFNG + S100A9 + CX3CR1 + CIITA | 0.803 | 0.705 | 0.9 |
| D 5/7 | CD177 + C3AR1 + IFNG + S100A9 + CX3CR1 + CIITA + CD3D | 0.791 | 0.678 | 0.903 |
| D 5/7 | CD177 + C3AR1 + IFNG + S100A9 + CX3CR1 + CIITA + CD3D + CTLA4 | 0.789 | 0.676 | 0.902 |
| D 5/7 | CD177 + C3AR1 + IFNG + S100A9 + CX3CR1 + CIITA + CTLA4 | 0.792 | 0.681 | 0.903 |
| D 5/7 | CD177 + C3AR1 + IFNG + S100A9 + CX3CR1 + CIITA + TAP2 | 0.808 | 0.714 | 0.902 |
| D 5/7 | CD177 + C3AR1 + IFNG + S100A9 + CX3CR1 + CIITA + TAP2 + CD3D | 0.808 | 0.708 | 0.907 |
| D 5/7 | CD177 + C3AR1 + IFNG + S100A9 + CX3CR1 + CIITA + TAP2 + CD3D + CTLA4 | 0.806 | 0.709 | 0.903 |
| D 5/7 | CD177 + C3AR1 + IFNG + S100A9 + CX3CR1 + CIITA + TAP2 + CTLA4 | 0.809 | 0.716 | 0.903 |
| D 5/7 | CD177 + C3AR1 + IFNG + S100A9 + CX3CR1 + CTLA4 | 0.783 | 0.663 | 0.902 |
| D 5/7 | CD177 + C3AR1 + IFNG + S100A9 + CX3CR1 + TAP2 | 0.808 | 0.713 | 0.902 |
| D 5/7 | CD177 + C3AR1 + IFNG + S100A9 + CX3CR1 + TAP2 + CD3D | 0.804 | 0.701 | 0.908 |
| D 5/7 | CD177 + C3AR1 + IFNG + S100A9 + CX3CR1 + TAP2 + CD3D + CTLA4 | 0.802 | 0.703 | 0.902 |
| D 5/7 | CD177 + C3AR1 + IFNG + S100A9 + CX3CR1 + TAP2 + CTLA4 | 0.805 | 0.71 | 0.901 |
| D 5/7 | CD177 + C3AR1 + IFNG + S100A9 + TAP2 | 0.795 | 0.704 | 0.886 |
| D 5/7 | CD177 + C3AR1 + IFNG + S100A9 + TAP2 + CD3D | 0.798 | 0.7 | 0.896 |
| D 5/7 | CD177 + C3AR1 + IFNG + S100A9 + TAP2 + CD3D + CTLA4 | 0.798 | 0.702 | 0.894 |
| D 5/7 | CD177 + C3AR1 + IFNG + S100A9 + TAP2 + CTLA4 | 0.794 | 0.702 | 0.886 |
| D 5/7 | CD177 + C3AR1 + IFNG + TAP2 | 0.758 | 0.653 | 0.863 |
| D 5/7 | CD177 + C3AR1 + IFNG + TAP2 + CD3D | 0.753 | 0.646 | 0.86 |
| D 5/7 | CD177 + C3AR1 + IFNG + TAP2 + CD3D + CTLA4 | 0.755 | 0.649 | 0.862 |
| D 5/7 | CD177 + C3AR1 + IFNG + TAP2 + CTLA4 | 0.76 | 0.653 | 0.868 |
| D 5/7 | CD177 + C3AR1 + S100A9 | 0.756 | 0.661 | 0.851 |
| D 5/7 | CD177 + C3AR1 + S100A9 + CD3D | 0.763 | 0.656 | 0.871 |
| D 5/7 | CD177 + C3AR1 + S100A9 + CD3D + CTLA4 | 0.76 | 0.648 | 0.871 |
| D 5/7 | CD177 + C3AR1 + S100A9 + CIITA | 0.762 | 0.671 | 0.854 |
| D 5/7 | CD177 + C3AR1 + S100A9 + CIITA + CD3D | 0.769 | 0.667 | 0.872 |
| D 5/7 | CD177 + C3AR1 + S100A9 + CIITA + CD3D + CTLA4 | 0.762 | 0.655 | 0.868 |
| D 5/7 | CD177 + C3AR1 + S100A9 + CIITA + CTLA4 | 0.742 | 0.635 | 0.849 |
| D 5/7 | CD177 + C3AR1 + S100A9 + CIITA + TAP2 | 0.789 | 0.704 | 0.873 |
| D 5/7 | CD177 + C3AR1 + S100A9 + CIITA + TAP2 + CD3D | 0.779 | 0.691 | 0.867 |
| D 5/7 | CD177 + C3AR1 + S100A9 + CIITA + TAP2 + CD3D + CTLA4 | 0.781 | 0.695 | 0.868 |
| D 5/7 | CD177 + C3AR1 + S100A9 + CIITA + TAP2 + CTLA4 | 0.785 | 0.699 | 0.871 |
| D 5/7 | CD177 + C3AR1 + S100A9 + CTLA4 | 0.741 | 0.632 | 0.85 |
| D 5/7 | CD177 + C3AR1 + S100A9 + CX3CR1 | 0.787 | 0.69 | 0.885 |
| D 5/7 | CD177 + C3AR1 + S100A9 + CX3CR1 + CD3D | 0.78 | 0.675 | 0.886 |
| D 5/7 | CD177 + C3AR1 + S100A9 + CX3CR1 + CD3D + CTLA4 | 0.775 | 0.664 | 0.887 |
| D 5/7 | CD177 + C3AR1 + S100A9 + CX3CR1 + CIITA | 0.786 | 0.691 | 0.882 |
| D 5/7 | CD177 + C3AR1 + S100A9 + CX3CR1 + CIITA + CD3D | 0.782 | 0.68 | 0.884 |
| D 5/7 | CD177 + C3AR1 + S100A9 + CX3CR1 + CIITA + CD3D + CTLA4 | 0.774 | 0.665 | 0.883 |
| D 5/7 | CD177 + C3AR1 + S100A9 + CX3CR1 + CIITA + CTLA4 | 0.778 | 0.668 | 0.889 |
| D 5/7 | CD177 + C3AR1 + S100A9 + CX3CR1 + CIITA + TAP2 | 0.79 | 0.704 | 0.875 |
| D 5/7 | CD177 + C3AR1 + S100A9 + CX3CR1 + CIITA + TAP2 + CD3D | 0.776 | 0.684 | 0.868 |
| D 5/7 | CD177 + C3AR1 + S100A9 + CX3CR1 + CIITA + TAP2 + CD3D + CTLA4 | 0.775 | 0.684 | 0.867 |
| D 5/7 | CD177 + C3AR1 + S100A9 + CX3CR1 + CIITA + TAP2 + CTLA4 | 0.789 | 0.7 | 0.878 |
| D 5/7 | CD177 + C3AR1 + S100A9 + CX3CR1 + CTLA4 | 0.778 | 0.666 | 0.89 |
| D 5/7 | CD177 + C3AR1 + S100A9 + CX3CR1 + TAP2 | 0.792 | 0.707 | 0.876 |
| D 5/7 | CD177 + C3AR1 + S100A9 + CX3CR1 + TAP2 + CD3D | 0.777 | 0.688 | 0.865 |
| D 5/7 | CD177 + C3AR1 + S100A9 + CX3CR1 + TAP2 + CD3D + CTLA4 | 0.777 | 0.688 | 0.866 |
| D 5/7 | CD177 + C3AR1 + S100A9 + CX3CR1 + TAP2 + CTLA4 | 0.79 | 0.704 | 0.875 |
| D 5/7 | CD177 + C3AR1 + S100A9 + TAP2 | 0.786 | 0.703 | 0.869 |
| D 5/7 | CD177 + C3AR1 + S100A9 + TAP2 + CD3D | 0.778 | 0.69 | 0.865 |
| D 5/7 | CD177 + C3AR1 + S100A9 + TAP2 + CD3D + CTLA4 | 0.778 | 0.691 | 0.864 |
| D 5/7 | CD177 + C3AR1 + S100A9 + TAP2 + CTLA4 | 0.788 | 0.703 | 0.873 |
| D 5/7 | CD177 + C3AR1 + TAP2 | 0.769 | 0.676 | 0.862 |
| D 5/7 | CD177 + C3AR1 + TAP2 + CD3D | 0.771 | 0.68 | 0.862 |
| D 5/7 | CD177 + C3AR1 + TAP2 + CD3D + CTLA4 | 0.769 | 0.676 | 0.863 |
| D 5/7 | CD177 + C3AR1 + TAP2 + CTLA4 | 0.769 | 0.676 | 0.863 |
| D 5/7 | CD177 + CD3D + CTLA4 | 0.699 | 0.577 | 0.821 |
| D 5/7 | CD177 + CD74 | 0.725 | 0.604 | 0.845 |
| D 5/7 | CD177 + CD74 + CD3D | 0.746 | 0.637 | 0.855 |
| D 5/7 | CD177 + CD74 + CD3D + CTLA4 | 0.742 | 0.631 | 0.852 |
| D 5/7 | CD177 + CD74 + CIITA | 0.723 | 0.599 | 0.848 |
| D 5/7 | CD177 + CD74 + CIITA + CD3D | 0.731 | 0.618 | 0.844 |
| D 5/7 | CD177 + CD74 + CIITA + CD3D + CTLA4 | 0.733 | 0.617 | 0.848 |
| D 5/7 | CD177 + CD74 + CIITA + CTLA4 | 0.73 | 0.603 | 0.856 |
| D 5/7 | CD177 + CD74 + CIITA + TAP2 | 0.75 | 0.64 | 0.86 |
| D 5/7 | CD177 + CD74 + CIITA + TAP2 + CD3D | 0.755 | 0.65 | 0.861 |
| D 5/7 | CD177 + CD74 + CIITA + TAP2 + CD3D + CTLA4 | 0.755 | 0.65 | 0.861 |
| D 5/7 | CD177 + CD74 + CIITA + TAP2 + CTLA4 | 0.752 | 0.64 | 0.865 |
| D 5/7 | CD177 + CD74 + CTLA4 | 0.73 | 0.609 | 0.85 |
| D 5/7 | CD177 + CD74 + CX3CR1 | 0.742 | 0.627 | 0.856 |
| D 5/7 | CD177 + CD74 + CX3CR1 + CD3D | 0.746 | 0.634 | 0.859 |
| D 5/7 | CD177 + CD74 + CX3CR1 + CD3D + CTLA4 | 0.743 | 0.629 | 0.856 |
| D 5/7 | CD177 + CD74 + CX3CR1 + CIITA | 0.74 | 0.622 | 0.859 |
| D 5/7 | CD177 + CD74 + CX3CR1 + CIITA + CD3D | 0.736 | 0.62 | 0.852 |
| D 5/7 | CD177 + CD74 + CX3CR1 + CIITA + CD3D + CTLA4 | 0.739 | 0.62 | 0.858 |
| D 5/7 | CD177 + CD74 + CX3CR1 + CIITA + CTLA4 | 0.732 | 0.608 | 0.856 |
| D 5/7 | CD177 + CD74 + CX3CR1 + CIITA + TAP2 | 0.758 | 0.65 | 0.866 |
| D 5/7 | CD177 + CD74 + CX3CR1 + CIITA + TAP2 + CD3D | 0.763 | 0.659 | 0.866 |
| D 5/7 | CD177 + CD74 + CX3CR1 + CIITA + TAP2 + CD3D + CTLA4 | 0.761 | 0.654 | 0.868 |
| D 5/7 | CD177 + CD74 + CX3CR1 + CIITA + TAP2 + CTLA4 | 0.756 | 0.644 | 0.868 |
| D 5/7 | CD177 + CD74 + CX3CR1 + CTLA4 | 0.742 | 0.624 | 0.86 |
| D 5/7 | CD177 + CD74 + CX3CR1 + TAP2 | 0.762 | 0.655 | 0.869 |
| D 5/7 | CD177 + CD74 + CX3CR1 + TAP2 + CD3D | 0.759 | 0.655 | 0.863 |
| D 5/7 | CD177 + CD74 + CX3CR1 + TAP2 + CD3D + CTLA4 | 0.762 | 0.658 | 0.866 |
| D 5/7 | CD177 + CD74 + CX3CR1 + TAP2 + CTLA4 | 0.758 | 0.65 | 0.867 |
| D 5/7 | CD177 + CD74 + IFNG | 0.737 | 0.613 | 0.86 |
| D 5/7 | CD177 + CD74 + IFNG + CD3D | 0.743 | 0.615 | 0.871 |
| D 5/7 | CD177 + CD74 + IFNG + CD3D + CTLA4 | 0.74 | 0.61 | 0.87 |
| D 5/7 | CD177 + CD74 + IFNG + CIITA | 0.735 | 0.612 | 0.858 |
| D 5/7 | CD177 + CD74 + IFNG + CIITA + CD3D | 0.747 | 0.62 | 0.874 |
| D 5/7 | CD177 + CD74 + IFNG + CIITA + CD3D + CTLA4 | 0.745 | 0.618 | 0.872 |
| D 5/7 | CD177 + CD74 + IFNG + CIITA + CTLA4 | 0.733 | 0.602 | 0.863 |
| D 5/7 | CD177 + CD74 + IFNG + CIITA + TAP2 | 0.769 | 0.657 | 0.881 |
| D 5/7 | CD177 + CD74 + IFNG + CIITA + TAP2 + CD3D | 0.756 | 0.641 | 0.871 |
| D 5/7 | CD177 + CD74 + IFNG + CIITA + TAP2 + CD3D + CTLA4 | 0.76 | 0.647 | 0.874 |
| D 5/7 | CD177 + CD74 + IFNG + CIITA + TAP2 + CTLA4 | 0.76 | 0.645 | 0.875 |
| D 5/7 | CD177 + CD74 + IFNG + CTLA4 | 0.735 | 0.604 | 0.865 |
| D 5/7 | CD177 + CD74 + IFNG + CX3CR1 | 0.746 | 0.619 | 0.873 |
| D 5/7 | CD177 + CD74 + IFNG + CX3CR1 + CD3D | 0.749 | 0.619 | 0.88 |
| D 5/7 | CD177 + CD74 + IFNG + CX3CR1 + CD3D + CTLA4 | 0.747 | 0.615 | 0.879 |
| D 5/7 | CD177 + CD74 + IFNG + CX3CR1 + CIITA | 0.754 | 0.631 | 0.876 |
| D 5/7 | CD177 + CD74 + IFNG + CX3CR1 + CIITA + CD3D | 0.752 | 0.625 | 0.88 |
| D 5/7 | CD177 + CD74 + IFNG + CX3CR1 + CIITA + CD3D + CTLA4 | 0.748 | 0.617 | 0.878 |
| D 5/7 | CD177 + CD74 + IFNG + CX3CR1 + CIITA + CTLA4 | 0.749 | 0.619 | 0.878 |
| D 5/7 | CD177 + CD74 + IFNG + CX3CR1 + CIITA + TAP2 | 0.772 | 0.66 | 0.884 |
| D 5/7 | CD177 + CD74 + IFNG + CX3CR1 + CIITA + TAP2 + CD3D | 0.762 | 0.647 | 0.878 |
| D 5/7 | CD177 + CD74 + IFNG + CX3CR1 + CIITA + TAP2 + CD3D + CTLA4 | 0.762 | 0.646 | 0.878 |
| D 5/7 | CD177 + CD74 + IFNG + CX3CR1 + CIITA + TAP2 + CTLA4 | 0.764 | 0.648 | 0.88 |
| D 5/7 | CD177 + CD74 + IFNG + CX3CR1 + CTLA4 | 0.747 | 0.615 | 0.878 |
| D 5/7 | CD177 + CD74 + IFNG + CX3CR1 + TAP2 | 0.77 | 0.657 | 0.884 |
| D 5/7 | CD177 + CD74 + IFNG + CX3CR1 + TAP2 + CD3D | 0.763 | 0.648 | 0.879 |
| D 5/7 | CD177 + CD74 + IFNG + CX3CR1 + TAP2 + CD3D + CTLA4 | 0.763 | 0.648 | 0.879 |
| D 5/7 | CD177 + CD74 + IFNG + CX3CR1 + TAP2 + CTLA4 | 0.764 | 0.646 | 0.882 |
| D 5/7 | CD177 + CD74 + IFNG + S100A9 | 0.761 | 0.653 | 0.868 |
| D 5/7 | CD177 + CD74 + IFNG + S100A9 + CD3D | 0.789 | 0.683 | 0.894 |
| D 5/7 | CD177 + CD74 + IFNG + S100A9 + CD3D + CTLA4 | 0.79 | 0.685 | 0.894 |
| D 5/7 | CD177 + CD74 + IFNG + S100A9 + CIITA | 0.758 | 0.644 | 0.872 |
| D 5/7 | CD177 + CD74 + IFNG + S100A9 + CIITA + CD3D | 0.789 | 0.681 | 0.897 |
| D 5/7 | CD177 + CD74 + IFNG + S100A9 + CIITA + CD3D + CTLA4 | 0.787 | 0.679 | 0.895 |
| D 5/7 | CD177 + CD74 + IFNG + S100A9 + CIITA + CTLA4 | 0.751 | 0.626 | 0.875 |
| D 5/7 | CD177 + CD74 + IFNG + S100A9 + CIITA + TAP2 | 0.789 | 0.691 | 0.886 |
| D 5/7 | CD177 + CD74 + IFNG + S100A9 + CIITA + TAP2 + CD3D | 0.796 | 0.697 | 0.894 |
| D 5/7 | CD177 + CD74 + IFNG + S100A9 + CIITA + TAP2 + CD3D + CTLA4 | 0.805 | 0.71 | 0.9 |
| D 5/7 | CD177 + CD74 + IFNG + S100A9 + CIITA + TAP2 + CTLA4 | 0.786 | 0.688 | 0.885 |
| D 5/7 | CD177 + CD74 + IFNG + S100A9 + CTLA4 | 0.764 | 0.651 | 0.878 |
| D 5/7 | CD177 + CD74 + IFNG + S100A9 + CX3CR1 | 0.804 | 0.705 | 0.902 |
| D 5/7 | CD177 + CD74 + IFNG + S100A9 + CX3CR1 + CD3D | 0.802 | 0.695 | 0.909 |
| D 5/7 | CD177 + CD74 + IFNG + S100A9 + CX3CR1 + CD3D + CTLA4 | 0.802 | 0.695 | 0.909 |
| D 5/7 | CD177 + CD74 + IFNG + S100A9 + CX3CR1 + CIITA | 0.8 | 0.698 | 0.902 |
| D 5/7 | CD177 + CD74 + IFNG + S100A9 + CX3CR1 + CIITA + CD3D | 0.8 | 0.692 | 0.908 |
| D 5/7 | CD177 + CD74 + IFNG + S100A9 + CX3CR1 + CIITA + CD3D + CTLA4 | 0.8 | 0.692 | 0.908 |
| D 5/7 | CD177 + CD74 + IFNG + S100A9 + CX3CR1 + CIITA + CTLA4 | 0.795 | 0.681 | 0.908 |
| D 5/7 | CD177 + CD74 + IFNG + S100A9 + CX3CR1 + CIITA + TAP2 | 0.813 | 0.716 | 0.909 |
| D 5/7 | CD177 + CD74 + IFNG + S100A9 + CX3CR1 + CIITA + TAP2 + CD3D | 0.807 | 0.708 | 0.907 |
| D 5/7 | CD177 + CD74 + IFNG + S100A9 + CX3CR1 + CIITA + TAP2 + CD3D + CTLA4 | 0.81 | 0.714 | 0.907 |
| D 5/7 | CD177 + CD74 + IFNG + S100A9 + CX3CR1 + CIITA + TAP2 + CTLA4 | 0.812 | 0.713 | 0.911 |
| D 5/7 | CD177 + CD74 + IFNG + S100A9 + CX3CR1 + CTLA4 | 0.799 | 0.692 | 0.906 |
| D 5/7 | CD177 + CD74 + IFNG + S100A9 + CX3CR1 + TAP2 | 0.817 | 0.724 | 0.911 |
| D 5/7 | CD177 + CD74 + IFNG + S100A9 + CX3CR1 + TAP2 + CD3D | 0.812 | 0.715 | 0.909 |
| D 5/7 | CD177 + CD74 + IFNG + S100A9 + CX3CR1 + TAP2 + CD3D + CTLA4 | 0.809 | 0.713 | 0.906 |
| D 5/7 | CD177 + CD74 + IFNG + S100A9 + CX3CR1 + TAP2 + CTLA4 | 0.82 | 0.726 | 0.913 |
| D 5/7 | CD177 + CD74 + IFNG + S100A9 + TAP2 | 0.798 | 0.705 | 0.891 |
| D 5/7 | CD177 + CD74 + IFNG + S100A9 + TAP2 + CD3D | 0.8 | 0.704 | 0.896 |
| D 5/7 | CD177 + CD74 + IFNG + S100A9 + TAP2 + CD3D + CTLA4 | 0.808 | 0.714 | 0.902 |
| D 5/7 | CD177 + CD74 + IFNG + S100A9 + TAP2 + CTLA4 | 0.796 | 0.701 | 0.89 |
| D 5/7 | CD177 + CD74 + IFNG + TAP2 | 0.766 | 0.654 | 0.879 |
| D 5/7 | CD177 + CD74 + IFNG + TAP2 + CD3D | 0.755 | 0.637 | 0.873 |
| D 5/7 | CD177 + CD74 + IFNG + TAP2 + CD3D + CTLA4 | 0.754 | 0.636 | 0.872 |
| D 5/7 | CD177 + CD74 + IFNG + TAP2 + CTLA4 | 0.76 | 0.644 | 0.876 |
| D 5/7 | CD177 + CD74 + S100A9 | 0.739 | 0.634 | 0.845 |
| D 5/7 | CD177 + CD74 + S100A9 + CD3D | 0.757 | 0.659 | 0.855 |
| D 5/7 | CD177 + CD74 + S100A9 + CD3D + CTLA4 | 0.758 | 0.66 | 0.856 |
| D 5/7 | CD177 + CD74 + S100A9 + CIITA | 0.75 | 0.639 | 0.861 |
| D 5/7 | CD177 + CD74 + S100A9 + CIITA + CD3D | 0.762 | 0.66 | 0.864 |
| D 5/7 | CD177 + CD74 + S100A9 + CIITA + CD3D + CTLA4 | 0.762 | 0.66 | 0.865 |
| D 5/7 | CD177 + CD74 + S100A9 + CIITA + CTLA4 | 0.737 | 0.617 | 0.856 |
| D 5/7 | CD177 + CD74 + S100A9 + CIITA + TAP2 | 0.765 | 0.667 | 0.863 |
| D 5/7 | CD177 + CD74 + S100A9 + CIITA + TAP2 + CD3D | 0.775 | 0.68 | 0.871 |
| D 5/7 | CD177 + CD74 + S100A9 + CIITA + TAP2 + CD3D + CTLA4 | 0.778 | 0.683 | 0.872 |
| D 5/7 | CD177 + CD74 + S100A9 + CIITA + TAP2 + CTLA4 | 0.768 | 0.668 | 0.868 |
| D 5/7 | CD177 + CD74 + S100A9 + CTLA4 | 0.742 | 0.636 | 0.847 |
| D 5/7 | CD177 + CD74 + S100A9 + CX3CR1 | 0.769 | 0.671 | 0.868 |
| D 5/7 | CD177 + CD74 + S100A9 + CX3CR1 + CD3D | 0.766 | 0.667 | 0.864 |
| D 5/7 | CD177 + CD74 + S100A9 + CX3CR1 + CD3D + CTLA4 | 0.764 | 0.666 | 0.863 |
| D 5/7 | CD177 + CD74 + S100A9 + CX3CR1 + CIITA | 0.777 | 0.673 | 0.881 |
| D 5/7 | CD177 + CD74 + S100A9 + CX3CR1 + CIITA + CD3D | 0.767 | 0.663 | 0.87 |
| D 5/7 | CD177 + CD74 + S100A9 + CX3CR1 + CIITA + CD3D + CTLA4 | 0.768 | 0.662 | 0.873 |
| D 5/7 | CD177 + CD74 + S100A9 + CX3CR1 + CIITA + CTLA4 | 0.768 | 0.653 | 0.883 |
| D 5/7 | CD177 + CD74 + S100A9 + CX3CR1 + CIITA + TAP2 | 0.778 | 0.682 | 0.875 |
| D 5/7 | CD177 + CD74 + S100A9 + CX3CR1 + CIITA + TAP2 + CD3D | 0.782 | 0.686 | 0.878 |
| D 5/7 | CD177 + CD74 + S100A9 + CX3CR1 + CIITA + TAP2 + CD3D + CTLA4 | 0.783 | 0.687 | 0.878 |
| D 5/7 | CD177 + CD74 + S100A9 + CX3CR1 + CIITA + TAP2 + CTLA4 | 0.777 | 0.678 | 0.877 |
| D 5/7 | CD177 + CD74 + S100A9 + CX3CR1 + CTLA4 | 0.766 | 0.665 | 0.866 |
| D 5/7 | CD177 + CD74 + S100A9 + CX3CR1 + TAP2 | 0.771 | 0.678 | 0.864 |
| D 5/7 | CD177 + CD74 + S100A9 + CX3CR1 + TAP2 + CD3D | 0.777 | 0.687 | 0.866 |
| D 5/7 | CD177 + CD74 + S100A9 + CX3CR1 + TAP2 + CD3D + CTLA4 | 0.776 | 0.687 | 0.865 |
| D 5/7 | CD177 + CD74 + S100A9 + CX3CR1 + TAP2 + CTLA4 | 0.778 | 0.684 | 0.871 |
| D 5/7 | CD177 + CD74 + S100A9 + TAP2 | 0.77 | 0.677 | 0.863 |
| D 5/7 | CD177 + CD74 + S100A9 + TAP2 + CD3D | 0.773 | 0.683 | 0.862 |
| D 5/7 | CD177 + CD74 + S100A9 + TAP2 + CD3D + CTLA4 | 0.77 | 0.682 | 0.859 |
| D 5/7 | CD177 + CD74 + S100A9 + TAP2 + CTLA4 | 0.771 | 0.676 | 0.866 |
| D 5/7 | CD177 + CD74 + TAP2 | 0.754 | 0.645 | 0.862 |
| D 5/7 | CD177 + CD74 + TAP2 + CD3D | 0.756 | 0.652 | 0.861 |
| D 5/7 | CD177 + CD74 + TAP2 + CD3D + CTLA4 | 0.758 | 0.655 | 0.861 |
| D 5/7 | CD177 + CD74 + TAP2 + CTLA4 | 0.756 | 0.646 | 0.867 |
| D 5/7 | CD177 + CIITA + CD3D | 0.731 | 0.627 | 0.836 |
| D 5/7 | CD177 + CIITA + CD3D + CTLA4 | 0.726 | 0.619 | 0.834 |
| D 5/7 | CD177 + CIITA + CTLA4 | 0.712 | 0.591 | 0.833 |
| D 5/7 | CD177 + CIITA + TAP2 | 0.76 | 0.664 | 0.856 |
| D 5/7 | CD177 + CIITA + TAP2 + CD3D | 0.758 | 0.664 | 0.852 |
| D 5/7 | CD177 + CIITA + TAP2 + CD3D + CTLA4 | 0.76 | 0.665 | 0.855 |
| D 5/7 | CD177 + CIITA + TAP2 + CTLA4 | 0.768 | 0.672 | 0.865 |
| D 5/7 | CD177 + CX3CR1 + CD3D | 0.733 | 0.615 | 0.851 |
| D 5/7 | CD177 + CX3CR1 + CD3D + CTLA4 | 0.719 | 0.594 | 0.844 |
| D 5/7 | CD177 + CX3CR1 + CIITA | 0.741 | 0.632 | 0.85 |
| D 5/7 | CD177 + CX3CR1 + CIITA + CD3D | 0.748 | 0.643 | 0.853 |
| D 5/7 | CD177 + CX3CR1 + CIITA + CD3D + CTLA4 | 0.737 | 0.625 | 0.848 |
| D 5/7 | CD177 + CX3CR1 + CIITA + CTLA4 | 0.736 | 0.621 | 0.851 |
| D 5/7 | CD177 + CX3CR1 + CIITA + TAP2 | 0.761 | 0.665 | 0.857 |
| D 5/7 | CD177 + CX3CR1 + CIITA + TAP2 + CD3D | 0.761 | 0.665 | 0.856 |
| D 5/7 | CD177 + CX3CR1 + CIITA + TAP2 + CD3D + CTLA4 | 0.762 | 0.665 | 0.859 |
| D 5/7 | CD177 + CX3CR1 + CIITA + TAP2 + CTLA4 | 0.768 | 0.669 | 0.866 |
| D 5/7 | CD177 + CX3CR1 + CTLA4 | 0.719 | 0.591 | 0.848 |
| D 5/7 | CD177 + CX3CR1 + TAP2 | 0.753 | 0.654 | 0.852 |
| D 5/7 | CD177 + CX3CR1 + TAP2 + CD3D | 0.755 | 0.655 | 0.856 |
| D 5/7 | CD177 + CX3CR1 + TAP2 + CD3D + CTLA4 | 0.758 | 0.654 | 0.863 |
| D 5/7 | CD177 + CX3CR1 + TAP2 + CTLA4 | 0.762 | 0.659 | 0.866 |
| D 5/7 | CD177 + IFNG + CD3D | 0.714 | 0.587 | 0.84 |
| D 5/7 | CD177 + IFNG + CD3D + CTLA4 | 0.708 | 0.579 | 0.837 |
| D 5/7 | CD177 + IFNG + CIITA | 0.704 | 0.592 | 0.815 |
| D 5/7 | CD177 + IFNG + CIITA + CD3D | 0.746 | 0.632 | 0.86 |
| D 5/7 | CD177 + IFNG + CIITA + CD3D + CTLA4 | 0.742 | 0.626 | 0.858 |
| D 5/7 | CD177 + IFNG + CIITA + CTLA4 | 0.718 | 0.594 | 0.842 |
| D 5/7 | CD177 + IFNG + CIITA + TAP2 | 0.784 | 0.687 | 0.882 |
| D 5/7 | CD177 + IFNG + CIITA + TAP2 + CD3D | 0.776 | 0.674 | 0.878 |
| D 5/7 | CD177 + IFNG + CIITA + TAP2 + CD3D + CTLA4 | 0.776 | 0.673 | 0.879 |
| D 5/7 | CD177 + IFNG + CIITA + TAP2 + CTLA4 | 0.768 | 0.664 | 0.872 |
| D 5/7 | CD177 + IFNG + CTLA4 | 0.688 | 0.548 | 0.827 |
| D 5/7 | CD177 + IFNG + CX3CR1 | 0.741 | 0.623 | 0.859 |
| D 5/7 | CD177 + IFNG + CX3CR1 + CD3D | 0.729 | 0.602 | 0.857 |
| D 5/7 | CD177 + IFNG + CX3CR1 + CD3D + CTLA4 | 0.719 | 0.587 | 0.85 |
| D 5/7 | CD177 + IFNG + CX3CR1 + CIITA | 0.764 | 0.66 | 0.868 |
| D 5/7 | CD177 + IFNG + CX3CR1 + CIITA + CD3D | 0.765 | 0.648 | 0.882 |
| D 5/7 | CD177 + IFNG + CX3CR1 + CIITA + CD3D + CTLA4 | 0.755 | 0.637 | 0.874 |
| D 5/7 | CD177 + IFNG + CX3CR1 + CIITA + CTLA4 | 0.748 | 0.63 | 0.866 |
| D 5/7 | CD177 + IFNG + CX3CR1 + CIITA + TAP2 | 0.787 | 0.689 | 0.885 |
| D 5/7 | CD177 + IFNG + CX3CR1 + CIITA + TAP2 + CD3D | 0.783 | 0.679 | 0.887 |
| D 5/7 | CD177 + IFNG + CX3CR1 + CIITA + TAP2 + CD3D + CTLA4 | 0.779 | 0.674 | 0.885 |
| D 5/7 | CD177 + IFNG + CX3CR1 + CIITA + TAP2 + CTLA4 | 0.781 | 0.677 | 0.884 |
| D 5/7 | CD177 + IFNG + CX3CR1 + CTLA4 | 0.716 | 0.583 | 0.848 |
| D 5/7 | CD177 + IFNG + CX3CR1 + TAP2 | 0.769 | 0.664 | 0.873 |
| D 5/7 | CD177 + IFNG + CX3CR1 + TAP2 + CD3D | 0.765 | 0.655 | 0.874 |
| D 5/7 | CD177 + IFNG + CX3CR1 + TAP2 + CD3D + CTLA4 | 0.762 | 0.651 | 0.873 |
| D 5/7 | CD177 + IFNG + CX3CR1 + TAP2 + CTLA4 | 0.762 | 0.649 | 0.874 |
| D 5/7 | CD177 + IFNG + S100A9 | 0.733 | 0.624 | 0.843 |
| D 5/7 | CD177 + IFNG + S100A9 + CD3D | 0.778 | 0.663 | 0.892 |
| D 5/7 | CD177 + IFNG + S100A9 + CD3D + CTLA4 | 0.775 | 0.659 | 0.892 |
| D 5/7 | CD177 + IFNG + S100A9 + CIITA | 0.749 | 0.65 | 0.847 |
| D 5/7 | CD177 + IFNG + S100A9 + CIITA + CD3D | 0.779 | 0.668 | 0.889 |
| D 5/7 | CD177 + IFNG + S100A9 + CIITA + CD3D + CTLA4 | 0.779 | 0.669 | 0.889 |
| D 5/7 | CD177 + IFNG + S100A9 + CIITA + CTLA4 | 0.74 | 0.62 | 0.86 |
| D 5/7 | CD177 + IFNG + S100A9 + CIITA + TAP2 | 0.809 | 0.72 | 0.897 |
| D 5/7 | CD177 + IFNG + S100A9 + CIITA + TAP2 + CD3D | 0.809 | 0.714 | 0.905 |
| D 5/7 | CD177 + IFNG + S100A9 + CIITA + TAP2 + CD3D + CTLA4 | 0.811 | 0.717 | 0.904 |
| D 5/7 | CD177 + IFNG + S100A9 + CIITA + TAP2 + CTLA4 | 0.805 | 0.713 | 0.898 |
| D 5/7 | CD177 + IFNG + S100A9 + CTLA4 | 0.732 | 0.602 | 0.861 |
| D 5/7 | CD177 + IFNG + S100A9 + CX3CR1 | 0.807 | 0.703 | 0.912 |
| D 5/7 | CD177 + IFNG + S100A9 + CX3CR1 + CD3D | 0.797 | 0.679 | 0.916 |
| D 5/7 | CD177 + IFNG + S100A9 + CX3CR1 + CD3D + CTLA4 | 0.797 | 0.677 | 0.916 |
| D 5/7 | CD177 + IFNG + S100A9 + CX3CR1 + CIITA | 0.813 | 0.717 | 0.909 |
| D 5/7 | CD177 + IFNG + S100A9 + CX3CR1 + CIITA + CD3D | 0.809 | 0.702 | 0.916 |
| D 5/7 | CD177 + IFNG + S100A9 + CX3CR1 + CIITA + CD3D + CTLA4 | 0.805 | 0.694 | 0.916 |
| D 5/7 | CD177 + IFNG + S100A9 + CX3CR1 + CIITA + CTLA4 | 0.801 | 0.691 | 0.911 |
| D 5/7 | CD177 + IFNG + S100A9 + CX3CR1 + CIITA + TAP2 | 0.819 | 0.73 | 0.909 |
| D 5/7 | CD177 + IFNG + S100A9 + CX3CR1 + CIITA + TAP2 + CD3D | 0.818 | 0.719 | 0.916 |
| D 5/7 | CD177 + IFNG + S100A9 + CX3CR1 + CIITA + TAP2 + CD3D + CTLA4 | 0.819 | 0.723 | 0.916 |
| D 5/7 | CD177 + IFNG + S100A9 + CX3CR1 + CIITA + TAP2 + CTLA4 | 0.815 | 0.721 | 0.91 |
| D 5/7 | CD177 + IFNG + S100A9 + CX3CR1 + CTLA4 | 0.793 | 0.677 | 0.91 |
| D 5/7 | CD177 + IFNG + S100A9 + CX3CR1 + TAP2 | 0.818 | 0.729 | 0.908 |
| D 5/7 | CD177 + IFNG + S100A9 + CX3CR1 + TAP2 + CD3D | 0.814 | 0.713 | 0.916 |
| D 5/7 | CD177 + IFNG + S100A9 + CX3CR1 + TAP2 + CD3D + CTLA4 | 0.815 | 0.714 | 0.916 |
| D 5/7 | CD177 + IFNG + S100A9 + CX3CR1 + TAP2 + CTLA4 | 0.812 | 0.716 | 0.909 |
| D 5/7 | CD177 + IFNG + S100A9 + TAP2 | 0.809 | 0.72 | 0.898 |
| D 5/7 | CD177 + IFNG + S100A9 + TAP2 + CD3D | 0.805 | 0.706 | 0.905 |
| D 5/7 | CD177 + IFNG + S100A9 + TAP2 + CD3D + CTLA4 | 0.809 | 0.715 | 0.904 |
| D 5/7 | CD177 + IFNG + S100A9 + TAP2 + CTLA4 | 0.805 | 0.712 | 0.897 |
| D 5/7 | CD177 + IFNG + TAP2 | 0.767 | 0.663 | 0.871 |
| D 5/7 | CD177 + IFNG + TAP2 + CD3D | 0.757 | 0.647 | 0.868 |
| D 5/7 | CD177 + IFNG + TAP2 + CD3D + CTLA4 | 0.753 | 0.64 | 0.866 |
| D 5/7 | CD177 + IFNG + TAP2 + CTLA4 | 0.761 | 0.65 | 0.871 |
| D 5/7 | CD177 + IL1R2 | 0.72 | 0.595 | 0.844 |
| D 5/7 | CD177 + IL1R2 + C3AR1 | 0.773 | 0.673 | 0.872 |
| D 5/7 | CD177 + IL1R2 + C3AR1 + CD3D | 0.755 | 0.637 | 0.873 |
| D 5/7 | CD177 + IL1R2 + C3AR1 + CD3D + CTLA4 | 0.75 | 0.63 | 0.87 |
| D 5/7 | CD177 + IL1R2 + C3AR1 + CD74 | 0.766 | 0.655 | 0.876 |
| D 5/7 | CD177 + IL1R2 + C3AR1 + CD74 + CD3D | 0.765 | 0.655 | 0.875 |
| D 5/7 | CD177 + IL1R2 + C3AR1 + CD74 + CD3D + CTLA4 | 0.764 | 0.652 | 0.876 |
| D 5/7 | CD177 + IL1R2 + C3AR1 + CD74 + CIITA | 0.758 | 0.636 | 0.881 |
| D 5/7 | CD177 + IL1R2 + C3AR1 + CD74 + CIITA + CD3D | 0.758 | 0.639 | 0.877 |
| D 5/7 | CD177 + IL1R2 + C3AR1 + CD74 + CIITA + CD3D + CTLA4 | 0.758 | 0.638 | 0.879 |
| D 5/7 | CD177 + IL1R2 + C3AR1 + CD74 + CIITA + CTLA4 | 0.748 | 0.62 | 0.875 |
| D 5/7 | CD177 + IL1R2 + C3AR1 + CD74 + CIITA + TAP2 | 0.783 | 0.674 | 0.891 |
| D 5/7 | CD177 + IL1R2 + C3AR1 + CD74 + CIITA + TAP2 + CD3D | 0.773 | 0.663 | 0.883 |
| D 5/7 | CD177 + IL1R2 + C3AR1 + CD74 + CIITA + TAP2 + CD3D + CTLA4 | 0.775 | 0.665 | 0.885 |
| D 5/7 | CD177 + IL1R2 + C3AR1 + CD74 + CIITA + TAP2 + CTLA4 | 0.778 | 0.667 | 0.889 |
| D 5/7 | CD177 + IL1R2 + C3AR1 + CD74 + CTLA4 | 0.766 | 0.652 | 0.88 |
| D 5/7 | CD177 + IL1R2 + C3AR1 + CD74 + CX3CR1 | 0.769 | 0.66 | 0.879 |
| D 5/7 | CD177 + IL1R2 + C3AR1 + CD74 + CX3CR1 + CD3D | 0.767 | 0.655 | 0.88 |
| D 5/7 | CD177 + IL1R2 + C3AR1 + CD74 + CX3CR1 + CD3D + CTLA4 | 0.762 | 0.648 | 0.876 |
| D 5/7 | CD177 + IL1R2 + C3AR1 + CD74 + CX3CR1 + CIITA | 0.763 | 0.643 | 0.883 |
| D 5/7 | CD177 + IL1R2 + C3AR1 + CD74 + CX3CR1 + CIITA + CD3D | 0.758 | 0.639 | 0.877 |
| D 5/7 | CD177 + IL1R2 + C3AR1 + CD74 + CX3CR1 + CIITA + CD3D + CTLA4 | 0.76 | 0.638 | 0.882 |
| D 5/7 | CD177 + IL1R2 + C3AR1 + CD74 + CX3CR1 + CIITA + CTLA4 | 0.751 | 0.623 | 0.88 |
| D 5/7 | CD177 + IL1R2 + C3AR1 + CD74 + CX3CR1 + CIITA + TAP2 | 0.779 | 0.668 | 0.89 |
| D 5/7 | CD177 + IL1R2 + C3AR1 + CD74 + CX3CR1 + CIITA + TAP2 + CD3D | 0.771 | 0.66 | 0.882 |
| D 5/7 | CD177 + IL1R2 + C3AR1 + CD74 + CX3CR1 + CIITA + TAP2 + CD3D + CTLA4 | 0.771 | 0.66 | 0.882 |
| D 5/7 | CD177 + IL1R2 + C3AR1 + CD74 + CX3CR1 + CIITA + TAP2 + CTLA4 | 0.778 | 0.666 | 0.889 |
| D 5/7 | CD177 + IL1R2 + C3AR1 + CD74 + CX3CR1 + CTLA4 | 0.767 | 0.653 | 0.881 |
| D 5/7 | CD177 + IL1R2 + C3AR1 + CD74 + CX3CR1 + TAP2 | 0.78 | 0.679 | 0.881 |
| D 5/7 | CD177 + IL1R2 + C3AR1 + CD74 + CX3CR1 + TAP2 + CD3D | 0.777 | 0.675 | 0.88 |
| D 5/7 | CD177 + IL1R2 + C3AR1 + CD74 + CX3CR1 + TAP2 + CD3D + CTLA4 | 0.777 | 0.674 | 0.879 |
| D 5/7 | CD177 + IL1R2 + C3AR1 + CD74 + CX3CR1 + TAP2 + CTLA4 | 0.78 | 0.677 | 0.883 |
| D 5/7 | CD177 + IL1R2 + C3AR1 + CD74 + IFNG | 0.759 | 0.638 | 0.88 |
| D 5/7 | CD177 + IL1R2 + C3AR1 + CD74 + IFNG + CD3D | 0.758 | 0.629 | 0.886 |
| D 5/7 | CD177 + IL1R2 + C3AR1 + CD74 + IFNG + CD3D + CTLA4 | 0.756 | 0.627 | 0.886 |
| D 5/7 | CD177 + IL1R2 + C3AR1 + CD74 + IFNG + CIITA | 0.752 | 0.624 | 0.879 |
| D 5/7 | CD177 + IL1R2 + C3AR1 + CD74 + IFNG + CIITA + CD3D | 0.758 | 0.629 | 0.887 |
| D 5/7 | CD177 + IL1R2 + C3AR1 + CD74 + IFNG + CIITA + CD3D + CTLA4 | 0.756 | 0.625 | 0.886 |
| D 5/7 | CD177 + IL1R2 + C3AR1 + CD74 + IFNG + CIITA + CTLA4 | 0.753 | 0.621 | 0.885 |
| D 5/7 | CD177 + IL1R2 + C3AR1 + CD74 + IFNG + CIITA + TAP2 | 0.783 | 0.669 | 0.896 |
| D 5/7 | CD177 + IL1R2 + C3AR1 + CD74 + IFNG + CIITA + TAP2 + CD3D | 0.778 | 0.664 | 0.892 |
| D 5/7 | CD177 + IL1R2 + C3AR1 + CD74 + IFNG + CIITA + TAP2 + CD3D + CTLA4 | 0.782 | 0.67 | 0.894 |
| D 5/7 | CD177 + IL1R2 + C3AR1 + CD74 + IFNG + CIITA + TAP2 + CTLA4 | 0.779 | 0.664 | 0.894 |
| D 5/7 | CD177 + IL1R2 + C3AR1 + CD74 + IFNG + CTLA4 | 0.755 | 0.628 | 0.883 |
| D 5/7 | CD177 + IL1R2 + C3AR1 + CD74 + IFNG + CX3CR1 | 0.756 | 0.629 | 0.883 |
| D 5/7 | CD177 + IL1R2 + C3AR1 + CD74 + IFNG + CX3CR1 + CD3D | 0.76 | 0.631 | 0.889 |
| D 5/7 | CD177 + IL1R2 + C3AR1 + CD74 + IFNG + CX3CR1 + CD3D + CTLA4 | 0.755 | 0.62 | 0.889 |
| D 5/7 | CD177 + IL1R2 + C3AR1 + CD74 + IFNG + CX3CR1 + CIITA | 0.757 | 0.63 | 0.884 |
| D 5/7 | CD177 + IL1R2 + C3AR1 + CD74 + IFNG + CX3CR1 + CIITA + CD3D | 0.76 | 0.632 | 0.889 |
| D 5/7 | CD177 + IL1R2 + C3AR1 + CD74 + IFNG + CX3CR1 + CIITA + CD3D + CTLA4 | 0.754 | 0.621 | 0.887 |
| D 5/7 | CD177 + IL1R2 + C3AR1 + CD74 + IFNG + CX3CR1 + CIITA + CTLA4 | 0.758 | 0.628 | 0.889 |
| D 5/7 | CD177 + IL1R2 + C3AR1 + CD74 + IFNG + CX3CR1 + CIITA + TAP2 | 0.784 | 0.67 | 0.897 |
| D 5/7 | CD177 + IL1R2 + C3AR1 + CD74 + IFNG + CX3CR1 + CIITA + TAP2 + CD3D | 0.783 | 0.67 | 0.895 |
| D 5/7 | CD177 + IL1R2 + C3AR1 + CD74 + IFNG + CX3CR1 + CIITA + TAP2 + CD3D + CTLA4 | 0.781 | 0.668 | 0.895 |
| D 5/7 | CD177 + IL1R2 + C3AR1 + CD74 + IFNG + CX3CR1 + CIITA + TAP2 + CTLA4 | 0.78 | 0.665 | 0.894 |
| D 5/7 | CD177 + IL1R2 + C3AR1 + CD74 + IFNG + CX3CR1 + CTLA4 | 0.758 | 0.628 | 0.888 |
| D 5/7 | CD177 + IL1R2 + C3AR1 + CD74 + IFNG + CX3CR1 + TAP2 | 0.783 | 0.67 | 0.896 |
| D 5/7 | CD177 + IL1R2 + C3AR1 + CD74 + IFNG + CX3CR1 + TAP2 + CD3D | 0.783 | 0.67 | 0.895 |
| D 5/7 | CD177 + IL1R2 + C3AR1 + CD74 + IFNG + CX3CR1 + TAP2 + CD3D + CTLA4 | 0.781 | 0.668 | 0.895 |
| D 5/7 | CD177 + IL1R2 + C3AR1 + CD74 + IFNG + CX3CR1 + TAP2 + CTLA4 | 0.782 | 0.668 | 0.896 |
| D 5/7 | CD177 + IL1R2 + C3AR1 + CD74 + IFNG + S100A9 | 0.781 | 0.677 | 0.886 |
| D 5/7 | CD177 + IL1R2 + C3AR1 + CD74 + IFNG + S100A9 + CD3D | 0.791 | 0.685 | 0.896 |
| D 5/7 | CD177 + IL1R2 + C3AR1 + CD74 + IFNG + S100A9 + CD3D + CTLA4 | 0.791 | 0.686 | 0.896 |
| D 5/7 | CD177 + IL1R2 + C3AR1 + CD74 + IFNG + S100A9 + CIITA | 0.776 | 0.665 | 0.887 |
| D 5/7 | CD177 + IL1R2 + C3AR1 + CD74 + IFNG + S100A9 + CIITA + CD3D | 0.792 | 0.684 | 0.9 |
| D 5/7 | CD177 + IL1R2 + C3AR1 + CD74 + IFNG + S100A9 + CIITA + CD3D + CTLA4 | 0.789 | 0.68 | 0.897 |
| D 5/7 | CD177 + IL1R2 + C3AR1 + CD74 + IFNG + S100A9 + CIITA + CTLA4 | 0.769 | 0.649 | 0.89 |
| D 5/7 | CD177 + IL1R2 + C3AR1 + CD74 + IFNG + S100A9 + CIITA + TAP2 | 0.798 | 0.701 | 0.895 |
| D 5/7 | CD177 + IL1R2 + C3AR1 + CD74 + IFNG + S100A9 + CIITA + TAP2 + CD3D | 0.8 | 0.703 | 0.897 |
| D 5/7 | CD177 + IL1R2 + C3AR1 + CD74 + IFNG + S100A9 + CIITA + TAP2 + CD3D + CTLA4 | 0.807 | 0.712 | 0.902 |
| D 5/7 | CD177 + IL1R2 + C3AR1 + CD74 + IFNG + S100A9 + CIITA + TAP2 + CTLA4 | 0.799 | 0.701 | 0.897 |
| D 5/7 | CD177 + IL1R2 + C3AR1 + CD74 + IFNG + S100A9 + CTLA4 | 0.779 | 0.67 | 0.887 |
| D 5/7 | CD177 + IL1R2 + C3AR1 + CD74 + IFNG + S100A9 + CX3CR1 | 0.8 | 0.702 | 0.898 |
| D 5/7 | CD177 + IL1R2 + C3AR1 + CD74 + IFNG + S100A9 + CX3CR1 + CD3D | 0.801 | 0.694 | 0.907 |
| D 5/7 | CD177 + IL1R2 + C3AR1 + CD74 + IFNG + S100A9 + CX3CR1 + CD3D + CTLA4 | 0.801 | 0.694 | 0.907 |
| D 5/7 | CD177 + IL1R2 + C3AR1 + CD74 + IFNG + S100A9 + CX3CR1 + CIITA | 0.798 | 0.699 | 0.898 |
| D 5/7 | CD177 + IL1R2 + C3AR1 + CD74 + IFNG + S100A9 + CX3CR1 + CIITA + CD3D | 0.8 | 0.692 | 0.908 |
| D 5/7 | CD177 + IL1R2 + C3AR1 + CD74 + IFNG + S100A9 + CX3CR1 + CIITA + CD3D + CTLA4 | 0.801 | 0.693 | 0.908 |
| D 5/7 | CD177 + IL1R2 + C3AR1 + CD74 + IFNG + S100A9 + CX3CR1 + CIITA + CTLA4 | 0.795 | 0.683 | 0.907 |
| D 5/7 | CD177 + IL1R2 + C3AR1 + CD74 + IFNG + S100A9 + CX3CR1 + CIITA + TAP2 | 0.814 | 0.719 | 0.909 |
| D 5/7 | CD177 + IL1R2 + C3AR1 + CD74 + IFNG + S100A9 + CX3CR1 + CIITA + TAP2 + CD3D | 0.809 | 0.711 | 0.908 |
| D 5/7 | CD177 + IL1R2 + C3AR1 + CD74 + IFNG + S100A9 + CX3CR1 + CIITA + TAP2 + CD3D + CTLA4 | 0.81 | 0.714 | 0.907 |
| D 5/7 | CD177 + IL1R2 + C3AR1 + CD74 + IFNG + S100A9 + CX3CR1 + CIITA + TAP2 + CTLA4 | 0.813 | 0.715 | 0.911 |
| D 5/7 | CD177 + IL1R2 + C3AR1 + CD74 + IFNG + S100A9 + CX3CR1 + CTLA4 | 0.797 | 0.692 | 0.903 |
| D 5/7 | CD177 + IL1R2 + C3AR1 + CD74 + IFNG + S100A9 + CX3CR1 + TAP2 | 0.82 | 0.728 | 0.911 |
| D 5/7 | CD177 + IL1R2 + C3AR1 + CD74 + IFNG + S100A9 + CX3CR1 + TAP2 + CD3D | 0.812 | 0.715 | 0.909 |
| D 5/7 | CD177 + IL1R2 + C3AR1 + CD74 + IFNG + S100A9 + CX3CR1 + TAP2 + CD3D + CTLA4 | 0.809 | 0.713 | 0.906 |
| D 5/7 | CD177 + IL1R2 + C3AR1 + CD74 + IFNG + S100A9 + CX3CR1 + TAP2 + CTLA4 | 0.819 | 0.725 | 0.912 |
| D 5/7 | CD177 + IL1R2 + C3AR1 + CD74 + IFNG + S100A9 + TAP2 | 0.809 | 0.718 | 0.899 |
| D 5/7 | CD177 + IL1R2 + C3AR1 + CD74 + IFNG + S100A9 + TAP2 + CD3D | 0.803 | 0.707 | 0.898 |
| D 5/7 | CD177 + IL1R2 + C3AR1 + CD74 + IFNG + S100A9 + TAP2 + CD3D + CTLA4 | 0.81 | 0.715 | 0.904 |
| D 5/7 | CD177 + IL1R2 + C3AR1 + CD74 + IFNG + S100A9 + TAP2 + CTLA4 | 0.808 | 0.716 | 0.9 |
| D 5/7 | CD177 + IL1R2 + C3AR1 + CD74 + IFNG + TAP2 | 0.788 | 0.679 | 0.897 |
| D 5/7 | CD177 + IL1R2 + C3AR1 + CD74 + IFNG + TAP2 + CD3D | 0.779 | 0.665 | 0.892 |
| D 5/7 | CD177 + IL1R2 + C3AR1 + CD74 + IFNG + TAP2 + CD3D + CTLA4 | 0.782 | 0.67 | 0.894 |
| D 5/7 | CD177 + IL1R2 + C3AR1 + CD74 + IFNG + TAP2 + CTLA4 | 0.785 | 0.675 | 0.896 |
| D 5/7 | CD177 + IL1R2 + C3AR1 + CD74 + S100A9 | 0.77 | 0.67 | 0.871 |
| D 5/7 | CD177 + IL1R2 + C3AR1 + CD74 + S100A9 + CD3D | 0.777 | 0.681 | 0.873 |
| D 5/7 | CD177 + IL1R2 + C3AR1 + CD74 + S100A9 + CD3D + CTLA4 | 0.779 | 0.683 | 0.874 |
| D 5/7 | CD177 + IL1R2 + C3AR1 + CD74 + S100A9 + CIITA | 0.775 | 0.665 | 0.885 |
| D 5/7 | CD177 + IL1R2 + C3AR1 + CD74 + S100A9 + CIITA + CD3D | 0.781 | 0.677 | 0.886 |
| D 5/7 | CD177 + IL1R2 + C3AR1 + CD74 + S100A9 + CIITA + CD3D + CTLA4 | 0.777 | 0.67 | 0.884 |
| D 5/7 | CD177 + IL1R2 + C3AR1 + CD74 + S100A9 + CIITA + CTLA4 | 0.766 | 0.649 | 0.884 |
| D 5/7 | CD177 + IL1R2 + C3AR1 + CD74 + S100A9 + CIITA + TAP2 | 0.79 | 0.691 | 0.889 |
| D 5/7 | CD177 + IL1R2 + C3AR1 + CD74 + S100A9 + CIITA + TAP2 + CD3D | 0.789 | 0.693 | 0.884 |
| D 5/7 | CD177 + IL1R2 + C3AR1 + CD74 + S100A9 + CIITA + TAP2 + CD3D + CTLA4 | 0.79 | 0.695 | 0.885 |
| D 5/7 | CD177 + IL1R2 + C3AR1 + CD74 + S100A9 + CIITA + TAP2 + CTLA4 | 0.792 | 0.693 | 0.89 |
| D 5/7 | CD177 + IL1R2 + C3AR1 + CD74 + S100A9 + CTLA4 | 0.773 | 0.669 | 0.876 |
| D 5/7 | CD177 + IL1R2 + C3AR1 + CD74 + S100A9 + CX3CR1 | 0.791 | 0.7 | 0.882 |
| D 5/7 | CD177 + IL1R2 + C3AR1 + CD74 + S100A9 + CX3CR1 + CD3D | 0.786 | 0.689 | 0.883 |
| D 5/7 | CD177 + IL1R2 + C3AR1 + CD74 + S100A9 + CX3CR1 + CD3D + CTLA4 | 0.781 | 0.681 | 0.881 |
| D 5/7 | CD177 + IL1R2 + C3AR1 + CD74 + S100A9 + CX3CR1 + CIITA | 0.785 | 0.681 | 0.889 |
| D 5/7 | CD177 + IL1R2 + C3AR1 + CD74 + S100A9 + CX3CR1 + CIITA + CD3D | 0.784 | 0.681 | 0.888 |
| D 5/7 | CD177 + IL1R2 + C3AR1 + CD74 + S100A9 + CX3CR1 + CIITA + CD3D + CTLA4 | 0.779 | 0.672 | 0.886 |
| D 5/7 | CD177 + IL1R2 + C3AR1 + CD74 + S100A9 + CX3CR1 + CIITA + CTLA4 | 0.777 | 0.663 | 0.89 |
| D 5/7 | CD177 + IL1R2 + C3AR1 + CD74 + S100A9 + CX3CR1 + CIITA + TAP2 | 0.793 | 0.695 | 0.892 |
| D 5/7 | CD177 + IL1R2 + C3AR1 + CD74 + S100A9 + CX3CR1 + CIITA + TAP2 + CD3D | 0.787 | 0.692 | 0.883 |
| D 5/7 | CD177 + IL1R2 + C3AR1 + CD74 + S100A9 + CX3CR1 + CIITA + TAP2 + CD3D + CTLA4 | 0.791 | 0.698 | 0.885 |
| D 5/7 | CD177 + IL1R2 + C3AR1 + CD74 + S100A9 + CX3CR1 + CIITA + TAP2 + CTLA4 | 0.793 | 0.694 | 0.892 |
| D 5/7 | CD177 + IL1R2 + C3AR1 + CD74 + S100A9 + CX3CR1 + CTLA4 | 0.782 | 0.682 | 0.882 |
| D 5/7 | CD177 + IL1R2 + C3AR1 + CD74 + S100A9 + CX3CR1 + TAP2 | 0.793 | 0.703 | 0.884 |
| D 5/7 | CD177 + IL1R2 + C3AR1 + CD74 + S100A9 + CX3CR1 + TAP2 + CD3D | 0.792 | 0.704 | 0.881 |
| D 5/7 | CD177 + IL1R2 + C3AR1 + CD74 + S100A9 + CX3CR1 + TAP2 + CD3D + CTLA4 | 0.795 | 0.707 | 0.883 |
| D 5/7 | CD177 + IL1R2 + C3AR1 + CD74 + S100A9 + CX3CR1 + TAP2 + CTLA4 | 0.791 | 0.699 | 0.883 |
| D 5/7 | CD177 + IL1R2 + C3AR1 + CD74 + S100A9 + TAP2 | 0.791 | 0.699 | 0.883 |
| D 5/7 | CD177 + IL1R2 + C3AR1 + CD74 + S100A9 + TAP2 + CD3D | 0.792 | 0.703 | 0.881 |
| D 5/7 | CD177 + IL1R2 + C3AR1 + CD74 + S100A9 + TAP2 + CD3D + CTLA4 | 0.795 | 0.707 | 0.883 |
| D 5/7 | CD177 + IL1R2 + C3AR1 + CD74 + S100A9 + TAP2 + CTLA4 | 0.792 | 0.7 | 0.885 |
| D 5/7 | CD177 + IL1R2 + C3AR1 + CD74 + TAP2 | 0.78 | 0.679 | 0.881 |
| D 5/7 | CD177 + IL1R2 + C3AR1 + CD74 + TAP2 + CD3D | 0.779 | 0.677 | 0.881 |
| D 5/7 | CD177 + IL1R2 + C3AR1 + CD74 + TAP2 + CD3D + CTLA4 | 0.779 | 0.677 | 0.882 |
| D 5/7 | CD177 + IL1R2 + C3AR1 + CD74 + TAP2 + CTLA4 | 0.78 | 0.677 | 0.883 |
| D 5/7 | CD177 + IL1R2 + C3AR1 + CIITA | 0.769 | 0.673 | 0.866 |
| D 5/7 | CD177 + IL1R2 + C3AR1 + CIITA + CD3D | 0.76 | 0.652 | 0.867 |
| D 5/7 | CD177 + IL1R2 + C3AR1 + CIITA + CD3D + CTLA4 | 0.752 | 0.641 | 0.862 |
| D 5/7 | CD177 + IL1R2 + C3AR1 + CIITA + CTLA4 | 0.752 | 0.643 | 0.861 |
| D 5/7 | CD177 + IL1R2 + C3AR1 + CIITA + TAP2 | 0.788 | 0.694 | 0.882 |
| D 5/7 | CD177 + IL1R2 + C3AR1 + CIITA + TAP2 + CD3D | 0.784 | 0.69 | 0.878 |
| D 5/7 | CD177 + IL1R2 + C3AR1 + CIITA + TAP2 + CD3D + CTLA4 | 0.784 | 0.69 | 0.878 |
| D 5/7 | CD177 + IL1R2 + C3AR1 + CIITA + TAP2 + CTLA4 | 0.787 | 0.691 | 0.883 |
| D 5/7 | CD177 + IL1R2 + C3AR1 + CTLA4 | 0.748 | 0.63 | 0.866 |
| D 5/7 | CD177 + IL1R2 + C3AR1 + CX3CR1 | 0.775 | 0.67 | 0.879 |
| D 5/7 | CD177 + IL1R2 + C3AR1 + CX3CR1 + CD3D | 0.766 | 0.65 | 0.882 |
| D 5/7 | CD177 + IL1R2 + C3AR1 + CX3CR1 + CD3D + CTLA4 | 0.754 | 0.634 | 0.874 |
| D 5/7 | CD177 + IL1R2 + C3AR1 + CX3CR1 + CIITA | 0.776 | 0.68 | 0.872 |
| D 5/7 | CD177 + IL1R2 + C3AR1 + CX3CR1 + CIITA + CD3D | 0.769 | 0.666 | 0.871 |
| D 5/7 | CD177 + IL1R2 + C3AR1 + CX3CR1 + CIITA + CD3D + CTLA4 | 0.76 | 0.649 | 0.87 |
| D 5/7 | CD177 + IL1R2 + C3AR1 + CX3CR1 + CIITA + CTLA4 | 0.761 | 0.651 | 0.872 |
| D 5/7 | CD177 + IL1R2 + C3AR1 + CX3CR1 + CIITA + TAP2 | 0.788 | 0.693 | 0.883 |
| D 5/7 | CD177 + IL1R2 + C3AR1 + CX3CR1 + CIITA + TAP2 + CD3D | 0.786 | 0.691 | 0.88 |
| D 5/7 | CD177 + IL1R2 + C3AR1 + CX3CR1 + CIITA + TAP2 + CD3D + CTLA4 | 0.785 | 0.691 | 0.88 |
| D 5/7 | CD177 + IL1R2 + C3AR1 + CX3CR1 + CIITA + TAP2 + CTLA4 | 0.786 | 0.69 | 0.882 |
| D 5/7 | CD177 + IL1R2 + C3AR1 + CX3CR1 + CTLA4 | 0.758 | 0.638 | 0.879 |
| D 5/7 | CD177 + IL1R2 + C3AR1 + CX3CR1 + TAP2 | 0.786 | 0.691 | 0.882 |
| D 5/7 | CD177 + IL1R2 + C3AR1 + CX3CR1 + TAP2 + CD3D | 0.783 | 0.689 | 0.877 |
| D 5/7 | CD177 + IL1R2 + C3AR1 + CX3CR1 + TAP2 + CD3D + CTLA4 | 0.783 | 0.689 | 0.878 |
| D 5/7 | CD177 + IL1R2 + C3AR1 + CX3CR1 + TAP2 + CTLA4 | 0.787 | 0.691 | 0.883 |
| D 5/7 | CD177 + IL1R2 + C3AR1 + IFNG | 0.736 | 0.613 | 0.86 |
| D 5/7 | CD177 + IL1R2 + C3AR1 + IFNG + CD3D | 0.727 | 0.592 | 0.862 |
| D 5/7 | CD177 + IL1R2 + C3AR1 + IFNG + CD3D + CTLA4 | 0.721 | 0.588 | 0.855 |
| D 5/7 | CD177 + IL1R2 + C3AR1 + IFNG + CIITA | 0.753 | 0.648 | 0.858 |
| D 5/7 | CD177 + IL1R2 + C3AR1 + IFNG + CIITA + CD3D | 0.751 | 0.632 | 0.869 |
| D 5/7 | CD177 + IL1R2 + C3AR1 + IFNG + CIITA + CD3D + CTLA4 | 0.751 | 0.636 | 0.867 |
| D 5/7 | CD177 + IL1R2 + C3AR1 + IFNG + CIITA + CTLA4 | 0.745 | 0.632 | 0.858 |
| D 5/7 | CD177 + IL1R2 + C3AR1 + IFNG + CIITA + TAP2 | 0.789 | 0.687 | 0.89 |
| D 5/7 | CD177 + IL1R2 + C3AR1 + IFNG + CIITA + TAP2 + CD3D | 0.78 | 0.675 | 0.886 |
| D 5/7 | CD177 + IL1R2 + C3AR1 + IFNG + CIITA + TAP2 + CD3D + CTLA4 | 0.781 | 0.678 | 0.885 |
| D 5/7 | CD177 + IL1R2 + C3AR1 + IFNG + CIITA + TAP2 + CTLA4 | 0.788 | 0.686 | 0.89 |
| D 5/7 | CD177 + IL1R2 + C3AR1 + IFNG + CTLA4 | 0.726 | 0.597 | 0.854 |
| D 5/7 | CD177 + IL1R2 + C3AR1 + IFNG + CX3CR1 | 0.748 | 0.626 | 0.869 |
| D 5/7 | CD177 + IL1R2 + C3AR1 + IFNG + CX3CR1 + CD3D | 0.739 | 0.607 | 0.871 |
| D 5/7 | CD177 + IL1R2 + C3AR1 + IFNG + CX3CR1 + CD3D + CTLA4 | 0.732 | 0.599 | 0.864 |
| D 5/7 | CD177 + IL1R2 + C3AR1 + IFNG + CX3CR1 + CIITA | 0.765 | 0.663 | 0.867 |
| D 5/7 | CD177 + IL1R2 + C3AR1 + IFNG + CX3CR1 + CIITA + CD3D | 0.765 | 0.647 | 0.883 |
| D 5/7 | CD177 + IL1R2 + C3AR1 + IFNG + CX3CR1 + CIITA + CD3D + CTLA4 | 0.764 | 0.649 | 0.88 |
| D 5/7 | CD177 + IL1R2 + C3AR1 + IFNG + CX3CR1 + CIITA + CTLA4 | 0.757 | 0.642 | 0.872 |
| D 5/7 | CD177 + IL1R2 + C3AR1 + IFNG + CX3CR1 + CIITA + TAP2 | 0.789 | 0.689 | 0.889 |
| D 5/7 | CD177 + IL1R2 + C3AR1 + IFNG + CX3CR1 + CIITA + TAP2 + CD3D | 0.783 | 0.679 | 0.886 |
| D 5/7 | CD177 + IL1R2 + C3AR1 + IFNG + CX3CR1 + CIITA + TAP2 + CD3D + CTLA4 | 0.783 | 0.68 | 0.885 |
| D 5/7 | CD177 + IL1R2 + C3AR1 + IFNG + CX3CR1 + CIITA + TAP2 + CTLA4 | 0.789 | 0.686 | 0.892 |
| D 5/7 | CD177 + IL1R2 + C3AR1 + IFNG + CX3CR1 + CTLA4 | 0.733 | 0.603 | 0.863 |
| D 5/7 | CD177 + IL1R2 + C3AR1 + IFNG + CX3CR1 + TAP2 | 0.784 | 0.681 | 0.887 |
| D 5/7 | CD177 + IL1R2 + C3AR1 + IFNG + CX3CR1 + TAP2 + CD3D | 0.769 | 0.657 | 0.882 |
| D 5/7 | CD177 + IL1R2 + C3AR1 + IFNG + CX3CR1 + TAP2 + CD3D + CTLA4 | 0.772 | 0.661 | 0.882 |
| D 5/7 | CD177 + IL1R2 + C3AR1 + IFNG + CX3CR1 + TAP2 + CTLA4 | 0.777 | 0.668 | 0.885 |
| D 5/7 | CD177 + IL1R2 + C3AR1 + IFNG + S100A9 | 0.757 | 0.648 | 0.866 |
| D 5/7 | CD177 + IL1R2 + C3AR1 + IFNG + S100A9 + CD3D | 0.77 | 0.652 | 0.888 |
| D 5/7 | CD177 + IL1R2 + C3AR1 + IFNG + S100A9 + CD3D + CTLA4 | 0.77 | 0.651 | 0.889 |
| D 5/7 | CD177 + IL1R2 + C3AR1 + IFNG + S100A9 + CIITA | 0.772 | 0.672 | 0.872 |
| D 5/7 | CD177 + IL1R2 + C3AR1 + IFNG + S100A9 + CIITA + CD3D | 0.771 | 0.659 | 0.883 |
| D 5/7 | CD177 + IL1R2 + C3AR1 + IFNG + S100A9 + CIITA + CD3D + CTLA4 | 0.769 | 0.657 | 0.882 |
| D 5/7 | CD177 + IL1R2 + C3AR1 + IFNG + S100A9 + CIITA + CTLA4 | 0.758 | 0.644 | 0.873 |
| D 5/7 | CD177 + IL1R2 + C3AR1 + IFNG + S100A9 + CIITA + TAP2 | 0.799 | 0.708 | 0.891 |
| D 5/7 | CD177 + IL1R2 + C3AR1 + IFNG + S100A9 + CIITA + TAP2 + CD3D | 0.798 | 0.7 | 0.896 |
| D 5/7 | CD177 + IL1R2 + C3AR1 + IFNG + S100A9 + CIITA + TAP2 + CD3D + CTLA4 | 0.8 | 0.705 | 0.895 |
| D 5/7 | CD177 + IL1R2 + C3AR1 + IFNG + S100A9 + CIITA + TAP2 + CTLA4 | 0.798 | 0.705 | 0.891 |
| D 5/7 | CD177 + IL1R2 + C3AR1 + IFNG + S100A9 + CTLA4 | 0.752 | 0.633 | 0.872 |
| D 5/7 | CD177 + IL1R2 + C3AR1 + IFNG + S100A9 + CX3CR1 | 0.791 | 0.683 | 0.899 |
| D 5/7 | CD177 + IL1R2 + C3AR1 + IFNG + S100A9 + CX3CR1 + CD3D | 0.778 | 0.657 | 0.899 |
| D 5/7 | CD177 + IL1R2 + C3AR1 + IFNG + S100A9 + CX3CR1 + CD3D + CTLA4 | 0.78 | 0.657 | 0.904 |
| D 5/7 | CD177 + IL1R2 + C3AR1 + IFNG + S100A9 + CX3CR1 + CIITA | 0.796 | 0.698 | 0.893 |
| D 5/7 | CD177 + IL1R2 + C3AR1 + IFNG + S100A9 + CX3CR1 + CIITA + CD3D | 0.79 | 0.679 | 0.901 |
| D 5/7 | CD177 + IL1R2 + C3AR1 + IFNG + S100A9 + CX3CR1 + CIITA + CD3D + CTLA4 | 0.789 | 0.676 | 0.902 |
| D 5/7 | CD177 + IL1R2 + C3AR1 + IFNG + S100A9 + CX3CR1 + CIITA + CTLA4 | 0.792 | 0.683 | 0.902 |
| D 5/7 | CD177 + IL1R2 + C3AR1 + IFNG + S100A9 + CX3CR1 + CIITA + TAP2 | 0.81 | 0.717 | 0.904 |
| D 5/7 | CD177 + IL1R2 + C3AR1 + IFNG + S100A9 + CX3CR1 + CIITA + TAP2 + CD3D | 0.805 | 0.706 | 0.905 |
| D 5/7 | CD177 + IL1R2 + C3AR1 + IFNG + S100A9 + CX3CR1 + CIITA + TAP2 + CD3D + CTLA4 | 0.807 | 0.708 | 0.905 |
| D 5/7 | CD177 + IL1R2 + C3AR1 + IFNG + S100A9 + CX3CR1 + CIITA + TAP2 + CTLA4 | 0.809 | 0.715 | 0.902 |
| D 5/7 | CD177 + IL1R2 + C3AR1 + IFNG + S100A9 + CX3CR1 + CTLA4 | 0.78 | 0.662 | 0.899 |
| D 5/7 | CD177 + IL1R2 + C3AR1 + IFNG + S100A9 + CX3CR1 + TAP2 | 0.81 | 0.716 | 0.904 |
| D 5/7 | CD177 + IL1R2 + C3AR1 + IFNG + S100A9 + CX3CR1 + TAP2 + CD3D | 0.804 | 0.701 | 0.908 |
| D 5/7 | CD177 + IL1R2 + C3AR1 + IFNG + S100A9 + CX3CR1 + TAP2 + CD3D + CTLA4 | 0.803 | 0.703 | 0.902 |
| D 5/7 | CD177 + IL1R2 + C3AR1 + IFNG + S100A9 + CX3CR1 + TAP2 + CTLA4 | 0.806 | 0.711 | 0.901 |
| D 5/7 | CD177 + IL1R2 + C3AR1 + IFNG + S100A9 + TAP2 | 0.802 | 0.711 | 0.893 |
| D 5/7 | CD177 + IL1R2 + C3AR1 + IFNG + S100A9 + TAP2 + CD3D | 0.799 | 0.702 | 0.897 |
| D 5/7 | CD177 + IL1R2 + C3AR1 + IFNG + S100A9 + TAP2 + CD3D + CTLA4 | 0.801 | 0.705 | 0.897 |
| D 5/7 | CD177 + IL1R2 + C3AR1 + IFNG + S100A9 + TAP2 + CTLA4 | 0.799 | 0.708 | 0.891 |
| D 5/7 | CD177 + IL1R2 + C3AR1 + IFNG + TAP2 | 0.776 | 0.67 | 0.882 |
| D 5/7 | CD177 + IL1R2 + C3AR1 + IFNG + TAP2 + CD3D | 0.772 | 0.663 | 0.882 |
| D 5/7 | CD177 + IL1R2 + C3AR1 + IFNG + TAP2 + CD3D + CTLA4 | 0.773 | 0.665 | 0.882 |
| D 5/7 | CD177 + IL1R2 + C3AR1 + IFNG + TAP2 + CTLA4 | 0.774 | 0.667 | 0.881 |
| D 5/7 | CD177 + IL1R2 + C3AR1 + S100A9 | 0.785 | 0.694 | 0.876 |
| D 5/7 | CD177 + IL1R2 + C3AR1 + S100A9 + CD3D | 0.768 | 0.657 | 0.88 |
| D 5/7 | CD177 + IL1R2 + C3AR1 + S100A9 + CD3D + CTLA4 | 0.764 | 0.649 | 0.88 |
| D 5/7 | CD177 + IL1R2 + C3AR1 + S100A9 + CIITA | 0.785 | 0.695 | 0.875 |
| D 5/7 | CD177 + IL1R2 + C3AR1 + S100A9 + CIITA + CD3D | 0.772 | 0.665 | 0.879 |
| D 5/7 | CD177 + IL1R2 + C3AR1 + S100A9 + CIITA + CD3D + CTLA4 | 0.765 | 0.654 | 0.876 |
| D 5/7 | CD177 + IL1R2 + C3AR1 + S100A9 + CIITA + CTLA4 | 0.763 | 0.65 | 0.876 |
| D 5/7 | CD177 + IL1R2 + C3AR1 + S100A9 + CIITA + TAP2 | 0.799 | 0.714 | 0.885 |
| D 5/7 | CD177 + IL1R2 + C3AR1 + S100A9 + CIITA + TAP2 + CD3D | 0.789 | 0.699 | 0.878 |
| D 5/7 | CD177 + IL1R2 + C3AR1 + S100A9 + CIITA + TAP2 + CD3D + CTLA4 | 0.79 | 0.701 | 0.878 |
| D 5/7 | CD177 + IL1R2 + C3AR1 + S100A9 + CIITA + TAP2 + CTLA4 | 0.799 | 0.712 | 0.886 |
| D 5/7 | CD177 + IL1R2 + C3AR1 + S100A9 + CTLA4 | 0.763 | 0.65 | 0.876 |
| D 5/7 | CD177 + IL1R2 + C3AR1 + S100A9 + CX3CR1 | 0.793 | 0.695 | 0.891 |
| D 5/7 | CD177 + IL1R2 + C3AR1 + S100A9 + CX3CR1 + CD3D | 0.781 | 0.673 | 0.89 |
| D 5/7 | CD177 + IL1R2 + C3AR1 + S100A9 + CX3CR1 + CD3D + CTLA4 | 0.775 | 0.662 | 0.888 |
| D 5/7 | CD177 + IL1R2 + C3AR1 + S100A9 + CX3CR1 + CIITA | 0.792 | 0.697 | 0.887 |
| D 5/7 | CD177 + IL1R2 + C3AR1 + S100A9 + CX3CR1 + CIITA + CD3D | 0.781 | 0.675 | 0.886 |
| D 5/7 | CD177 + IL1R2 + C3AR1 + S100A9 + CX3CR1 + CIITA + CD3D + CTLA4 | 0.778 | 0.667 | 0.889 |
| D 5/7 | CD177 + IL1R2 + C3AR1 + S100A9 + CX3CR1 + CIITA + CTLA4 | 0.782 | 0.669 | 0.895 |
| D 5/7 | CD177 + IL1R2 + C3AR1 + S100A9 + CX3CR1 + CIITA + TAP2 | 0.803 | 0.718 | 0.889 |
| D 5/7 | CD177 + IL1R2 + C3AR1 + S100A9 + CX3CR1 + CIITA + TAP2 + CD3D | 0.787 | 0.697 | 0.878 |
| D 5/7 | CD177 + IL1R2 + C3AR1 + S100A9 + CX3CR1 + CIITA + TAP2 + CD3D + CTLA4 | 0.79 | 0.701 | 0.878 |
| D 5/7 | CD177 + IL1R2 + C3AR1 + S100A9 + CX3CR1 + CIITA + TAP2 + CTLA4 | 0.797 | 0.709 | 0.885 |
| D 5/7 | CD177 + IL1R2 + C3AR1 + S100A9 + CX3CR1 + CTLA4 | 0.781 | 0.666 | 0.896 |
| D 5/7 | CD177 + IL1R2 + C3AR1 + S100A9 + CX3CR1 + TAP2 | 0.798 | 0.713 | 0.883 |
| D 5/7 | CD177 + IL1R2 + C3AR1 + S100A9 + CX3CR1 + TAP2 + CD3D | 0.787 | 0.699 | 0.876 |
| D 5/7 | CD177 + IL1R2 + C3AR1 + S100A9 + CX3CR1 + TAP2 + CD3D + CTLA4 | 0.79 | 0.703 | 0.876 |
| D 5/7 | CD177 + IL1R2 + C3AR1 + S100A9 + CX3CR1 + TAP2 + CTLA4 | 0.798 | 0.711 | 0.884 |
| D 5/7 | CD177 + IL1R2 + C3AR1 + S100A9 + TAP2 | 0.793 | 0.707 | 0.879 |
| D 5/7 | CD177 + IL1R2 + C3AR1 + S100A9 + TAP2 + CD3D | 0.785 | 0.696 | 0.873 |
| D 5/7 | CD177 + IL1R2 + C3AR1 + S100A9 + TAP2 + CD3D + CTLA4 | 0.791 | 0.704 | 0.877 |
| D 5/7 | CD177 + IL1R2 + C3AR1 + S100A9 + TAP2 + CTLA4 | 0.793 | 0.708 | 0.879 |
| D 5/7 | CD177 + IL1R2 + C3AR1 + TAP2 | 0.786 | 0.691 | 0.882 |
| D 5/7 | CD177 + IL1R2 + C3AR1 + TAP2 + CD3D | 0.785 | 0.69 | 0.879 |
| D 5/7 | CD177 + IL1R2 + C3AR1 + TAP2 + CD3D + CTLA4 | 0.785 | 0.69 | 0.88 |
| D 5/7 | CD177 + IL1R2 + C3AR1 + TAP2 + CTLA4 | 0.786 | 0.689 | 0.882 |
| D 5/7 | CD177 + IL1R2 + CD3D | 0.723 | 0.595 | 0.852 |
| D 5/7 | CD177 + IL1R2 + CD3D + CTLA4 | 0.716 | 0.583 | 0.849 |
| D 5/7 | CD177 + IL1R2 + CD74 | 0.74 | 0.625 | 0.855 |
| D 5/7 | CD177 + IL1R2 + CD74 + CD3D | 0.746 | 0.635 | 0.858 |
| D 5/7 | CD177 + IL1R2 + CD74 + CD3D + CTLA4 | 0.747 | 0.636 | 0.858 |
| D 5/7 | CD177 + IL1R2 + CD74 + CIITA | 0.733 | 0.608 | 0.859 |
| D 5/7 | CD177 + IL1R2 + CD74 + CIITA + CD3D | 0.742 | 0.623 | 0.861 |
| D 5/7 | CD177 + IL1R2 + CD74 + CIITA + CD3D + CTLA4 | 0.741 | 0.619 | 0.863 |
| D 5/7 | CD177 + IL1R2 + CD74 + CIITA + CTLA4 | 0.734 | 0.605 | 0.862 |
| D 5/7 | CD177 + IL1R2 + CD74 + CIITA + TAP2 | 0.764 | 0.652 | 0.876 |
| D 5/7 | CD177 + IL1R2 + CD74 + CIITA + TAP2 + CD3D | 0.761 | 0.647 | 0.874 |
| D 5/7 | CD177 + IL1R2 + CD74 + CIITA + TAP2 + CD3D + CTLA4 | 0.76 | 0.647 | 0.873 |
| D 5/7 | CD177 + IL1R2 + CD74 + CIITA + TAP2 + CTLA4 | 0.763 | 0.65 | 0.877 |
| D 5/7 | CD177 + IL1R2 + CD74 + CTLA4 | 0.737 | 0.619 | 0.854 |
| D 5/7 | CD177 + IL1R2 + CD74 + CX3CR1 | 0.749 | 0.635 | 0.862 |
| D 5/7 | CD177 + IL1R2 + CD74 + CX3CR1 + CD3D | 0.748 | 0.636 | 0.86 |
| D 5/7 | CD177 + IL1R2 + CD74 + CX3CR1 + CD3D + CTLA4 | 0.749 | 0.637 | 0.861 |
| D 5/7 | CD177 + IL1R2 + CD74 + CX3CR1 + CIITA | 0.75 | 0.629 | 0.872 |
| D 5/7 | CD177 + IL1R2 + CD74 + CX3CR1 + CIITA + CD3D | 0.743 | 0.623 | 0.863 |
| D 5/7 | CD177 + IL1R2 + CD74 + CX3CR1 + CIITA + CD3D + CTLA4 | 0.745 | 0.624 | 0.866 |
| D 5/7 | CD177 + IL1R2 + CD74 + CX3CR1 + CIITA + CTLA4 | 0.746 | 0.62 | 0.872 |
| D 5/7 | CD177 + IL1R2 + CD74 + CX3CR1 + CIITA + TAP2 | 0.764 | 0.651 | 0.877 |
| D 5/7 | CD177 + IL1R2 + CD74 + CX3CR1 + CIITA + TAP2 + CD3D | 0.762 | 0.651 | 0.873 |
| D 5/7 | CD177 + IL1R2 + CD74 + CX3CR1 + CIITA + TAP2 + CD3D + CTLA4 | 0.764 | 0.654 | 0.874 |
| D 5/7 | CD177 + IL1R2 + CD74 + CX3CR1 + CIITA + TAP2 + CTLA4 | 0.764 | 0.651 | 0.878 |
| D 5/7 | CD177 + IL1R2 + CD74 + CX3CR1 + CTLA4 | 0.744 | 0.63 | 0.859 |
| D 5/7 | CD177 + IL1R2 + CD74 + CX3CR1 + TAP2 | 0.768 | 0.662 | 0.874 |
| D 5/7 | CD177 + IL1R2 + CD74 + CX3CR1 + TAP2 + CD3D | 0.759 | 0.654 | 0.864 |
| D 5/7 | CD177 + IL1R2 + CD74 + CX3CR1 + TAP2 + CD3D + CTLA4 | 0.757 | 0.652 | 0.863 |
| D 5/7 | CD177 + IL1R2 + CD74 + CX3CR1 + TAP2 + CTLA4 | 0.767 | 0.66 | 0.874 |
| D 5/7 | CD177 + IL1R2 + CD74 + IFNG | 0.751 | 0.63 | 0.871 |
| D 5/7 | CD177 + IL1R2 + CD74 + IFNG + CD3D | 0.75 | 0.622 | 0.878 |
| D 5/7 | CD177 + IL1R2 + CD74 + IFNG + CD3D + CTLA4 | 0.75 | 0.622 | 0.879 |
| D 5/7 | CD177 + IL1R2 + CD74 + IFNG + CIITA | 0.751 | 0.626 | 0.876 |
| D 5/7 | CD177 + IL1R2 + CD74 + IFNG + CIITA + CD3D | 0.75 | 0.622 | 0.878 |
| D 5/7 | CD177 + IL1R2 + CD74 + IFNG + CIITA + CD3D + CTLA4 | 0.75 | 0.622 | 0.879 |
| D 5/7 | CD177 + IL1R2 + CD74 + IFNG + CIITA + CTLA4 | 0.748 | 0.616 | 0.879 |
| D 5/7 | CD177 + IL1R2 + CD74 + IFNG + CIITA + TAP2 | 0.774 | 0.66 | 0.889 |
| D 5/7 | CD177 + IL1R2 + CD74 + IFNG + CIITA + TAP2 + CD3D | 0.77 | 0.654 | 0.885 |
| D 5/7 | CD177 + IL1R2 + CD74 + IFNG + CIITA + TAP2 + CD3D + CTLA4 | 0.773 | 0.66 | 0.886 |
| D 5/7 | CD177 + IL1R2 + CD74 + IFNG + CIITA + TAP2 + CTLA4 | 0.771 | 0.654 | 0.888 |
| D 5/7 | CD177 + IL1R2 + CD74 + IFNG + CTLA4 | 0.746 | 0.617 | 0.876 |
| D 5/7 | CD177 + IL1R2 + CD74 + IFNG + CX3CR1 | 0.752 | 0.626 | 0.878 |
| D 5/7 | CD177 + IL1R2 + CD74 + IFNG + CX3CR1 + CD3D | 0.756 | 0.626 | 0.887 |
| D 5/7 | CD177 + IL1R2 + CD74 + IFNG + CX3CR1 + CD3D + CTLA4 | 0.75 | 0.618 | 0.883 |
| D 5/7 | CD177 + IL1R2 + CD74 + IFNG + CX3CR1 + CIITA | 0.751 | 0.624 | 0.877 |
| D 5/7 | CD177 + IL1R2 + CD74 + IFNG + CX3CR1 + CIITA + CD3D | 0.757 | 0.628 | 0.886 |
| D 5/7 | CD177 + IL1R2 + CD74 + IFNG + CX3CR1 + CIITA + CD3D + CTLA4 | 0.752 | 0.621 | 0.884 |
| D 5/7 | CD177 + IL1R2 + CD74 + IFNG + CX3CR1 + CIITA + CTLA4 | 0.752 | 0.622 | 0.882 |
| D 5/7 | CD177 + IL1R2 + CD74 + IFNG + CX3CR1 + CIITA + TAP2 | 0.774 | 0.661 | 0.888 |
| D 5/7 | CD177 + IL1R2 + CD74 + IFNG + CX3CR1 + CIITA + TAP2 + CD3D | 0.774 | 0.659 | 0.888 |
| D 5/7 | CD177 + IL1R2 + CD74 + IFNG + CX3CR1 + CIITA + TAP2 + CD3D + CTLA4 | 0.776 | 0.663 | 0.889 |
| D 5/7 | CD177 + IL1R2 + CD74 + IFNG + CX3CR1 + CIITA + TAP2 + CTLA4 | 0.774 | 0.659 | 0.89 |
| D 5/7 | CD177 + IL1R2 + CD74 + IFNG + CX3CR1 + CTLA4 | 0.752 | 0.622 | 0.882 |
| D 5/7 | CD177 + IL1R2 + CD74 + IFNG + CX3CR1 + TAP2 | 0.777 | 0.664 | 0.889 |
| D 5/7 | CD177 + IL1R2 + CD74 + IFNG + CX3CR1 + TAP2 + CD3D | 0.774 | 0.659 | 0.888 |
| D 5/7 | CD177 + IL1R2 + CD74 + IFNG + CX3CR1 + TAP2 + CD3D + CTLA4 | 0.776 | 0.663 | 0.889 |
| D 5/7 | CD177 + IL1R2 + CD74 + IFNG + CX3CR1 + TAP2 + CTLA4 | 0.778 | 0.664 | 0.893 |
| D 5/7 | CD177 + IL1R2 + CD74 + IFNG + S100A9 | 0.772 | 0.666 | 0.877 |
| D 5/7 | CD177 + IL1R2 + CD74 + IFNG + S100A9 + CD3D | 0.79 | 0.684 | 0.895 |
| D 5/7 | CD177 + IL1R2 + CD74 + IFNG + S100A9 + CD3D + CTLA4 | 0.791 | 0.686 | 0.896 |
| D 5/7 | CD177 + IL1R2 + CD74 + IFNG + S100A9 + CIITA | 0.77 | 0.656 | 0.885 |
| D 5/7 | CD177 + IL1R2 + CD74 + IFNG + S100A9 + CIITA + CD3D | 0.789 | 0.681 | 0.897 |
| D 5/7 | CD177 + IL1R2 + CD74 + IFNG + S100A9 + CIITA + CD3D + CTLA4 | 0.789 | 0.681 | 0.896 |
| D 5/7 | CD177 + IL1R2 + CD74 + IFNG + S100A9 + CIITA + CTLA4 | 0.761 | 0.636 | 0.885 |
| D 5/7 | CD177 + IL1R2 + CD74 + IFNG + S100A9 + CIITA + TAP2 | 0.793 | 0.694 | 0.893 |
| D 5/7 | CD177 + IL1R2 + CD74 + IFNG + S100A9 + CIITA + TAP2 + CD3D | 0.797 | 0.697 | 0.897 |
| D 5/7 | CD177 + IL1R2 + CD74 + IFNG + S100A9 + CIITA + TAP2 + CD3D + CTLA4 | 0.807 | 0.711 | 0.902 |
| D 5/7 | CD177 + IL1R2 + CD74 + IFNG + S100A9 + CIITA + TAP2 + CTLA4 | 0.793 | 0.693 | 0.893 |
| D 5/7 | CD177 + IL1R2 + CD74 + IFNG + S100A9 + CTLA4 | 0.772 | 0.661 | 0.883 |
| D 5/7 | CD177 + IL1R2 + CD74 + IFNG + S100A9 + CX3CR1 | 0.801 | 0.702 | 0.9 |
| D 5/7 | CD177 + IL1R2 + CD74 + IFNG + S100A9 + CX3CR1 + CD3D | 0.8 | 0.694 | 0.906 |
| D 5/7 | CD177 + IL1R2 + CD74 + IFNG + S100A9 + CX3CR1 + CD3D + CTLA4 | 0.8 | 0.694 | 0.906 |
| D 5/7 | CD177 + IL1R2 + CD74 + IFNG + S100A9 + CX3CR1 + CIITA | 0.798 | 0.696 | 0.899 |
| D 5/7 | CD177 + IL1R2 + CD74 + IFNG + S100A9 + CX3CR1 + CIITA + CD3D | 0.802 | 0.695 | 0.909 |
| D 5/7 | CD177 + IL1R2 + CD74 + IFNG + S100A9 + CX3CR1 + CIITA + CD3D + CTLA4 | 0.801 | 0.693 | 0.908 |
| D 5/7 | CD177 + IL1R2 + CD74 + IFNG + S100A9 + CX3CR1 + CIITA + CTLA4 | 0.795 | 0.682 | 0.907 |
| D 5/7 | CD177 + IL1R2 + CD74 + IFNG + S100A9 + CX3CR1 + CIITA + TAP2 | 0.813 | 0.716 | 0.91 |
| D 5/7 | CD177 + IL1R2 + CD74 + IFNG + S100A9 + CX3CR1 + CIITA + TAP2 + CD3D | 0.809 | 0.711 | 0.908 |
| D 5/7 | CD177 + IL1R2 + CD74 + IFNG + S100A9 + CX3CR1 + CIITA + TAP2 + CD3D + CTLA4 | 0.81 | 0.714 | 0.907 |
| D 5/7 | CD177 + IL1R2 + CD74 + IFNG + S100A9 + CX3CR1 + CIITA + TAP2 + CTLA4 | 0.812 | 0.713 | 0.911 |
| D 5/7 | CD177 + IL1R2 + CD74 + IFNG + S100A9 + CX3CR1 + CTLA4 | 0.797 | 0.692 | 0.903 |
| D 5/7 | CD177 + IL1R2 + CD74 + IFNG + S100A9 + CX3CR1 + TAP2 | 0.816 | 0.723 | 0.91 |
| D 5/7 | CD177 + IL1R2 + CD74 + IFNG + S100A9 + CX3CR1 + TAP2 + CD3D | 0.812 | 0.715 | 0.909 |
| D 5/7 | CD177 + IL1R2 + CD74 + IFNG + S100A9 + CX3CR1 + TAP2 + CD3D + CTLA4 | 0.809 | 0.712 | 0.905 |
| D 5/7 | CD177 + IL1R2 + CD74 + IFNG + S100A9 + CX3CR1 + TAP2 + CTLA4 | 0.819 | 0.724 | 0.913 |
| D 5/7 | CD177 + IL1R2 + CD74 + IFNG + S100A9 + TAP2 | 0.805 | 0.713 | 0.898 |
| D 5/7 | CD177 + IL1R2 + CD74 + IFNG + S100A9 + TAP2 + CD3D | 0.802 | 0.705 | 0.899 |
| D 5/7 | CD177 + IL1R2 + CD74 + IFNG + S100A9 + TAP2 + CD3D + CTLA4 | 0.809 | 0.715 | 0.904 |
| D 5/7 | CD177 + IL1R2 + CD74 + IFNG + S100A9 + TAP2 + CTLA4 | 0.804 | 0.711 | 0.897 |
| D 5/7 | CD177 + IL1R2 + CD74 + IFNG + TAP2 | 0.777 | 0.664 | 0.889 |
| D 5/7 | CD177 + IL1R2 + CD74 + IFNG + TAP2 + CD3D | 0.771 | 0.657 | 0.885 |
| D 5/7 | CD177 + IL1R2 + CD74 + IFNG + TAP2 + CD3D + CTLA4 | 0.775 | 0.663 | 0.887 |
| D 5/7 | CD177 + IL1R2 + CD74 + IFNG + TAP2 + CTLA4 | 0.774 | 0.661 | 0.888 |
| D 5/7 | CD177 + IL1R2 + CD74 + S100A9 | 0.754 | 0.649 | 0.859 |
| D 5/7 | CD177 + IL1R2 + CD74 + S100A9 + CD3D | 0.762 | 0.664 | 0.86 |
| D 5/7 | CD177 + IL1R2 + CD74 + S100A9 + CD3D + CTLA4 | 0.762 | 0.664 | 0.86 |
| D 5/7 | CD177 + IL1R2 + CD74 + S100A9 + CIITA | 0.761 | 0.647 | 0.874 |
| D 5/7 | CD177 + IL1R2 + CD74 + S100A9 + CIITA + CD3D | 0.767 | 0.659 | 0.876 |
| D 5/7 | CD177 + IL1R2 + CD74 + S100A9 + CIITA + CD3D + CTLA4 | 0.764 | 0.654 | 0.874 |
| D 5/7 | CD177 + IL1R2 + CD74 + S100A9 + CIITA + CTLA4 | 0.746 | 0.623 | 0.869 |
| D 5/7 | CD177 + IL1R2 + CD74 + S100A9 + CIITA + TAP2 | 0.78 | 0.68 | 0.881 |
| D 5/7 | CD177 + IL1R2 + CD74 + S100A9 + CIITA + TAP2 + CD3D | 0.781 | 0.682 | 0.88 |
| D 5/7 | CD177 + IL1R2 + CD74 + S100A9 + CIITA + TAP2 + CD3D + CTLA4 | 0.782 | 0.684 | 0.88 |
| D 5/7 | CD177 + IL1R2 + CD74 + S100A9 + CIITA + TAP2 + CTLA4 | 0.78 | 0.677 | 0.883 |
| D 5/7 | CD177 + IL1R2 + CD74 + S100A9 + CTLA4 | 0.749 | 0.645 | 0.854 |
| D 5/7 | CD177 + IL1R2 + CD74 + S100A9 + CX3CR1 | 0.77 | 0.672 | 0.869 |
| D 5/7 | CD177 + IL1R2 + CD74 + S100A9 + CX3CR1 + CD3D | 0.762 | 0.663 | 0.861 |
| D 5/7 | CD177 + IL1R2 + CD74 + S100A9 + CX3CR1 + CD3D + CTLA4 | 0.761 | 0.661 | 0.861 |
| D 5/7 | CD177 + IL1R2 + CD74 + S100A9 + CX3CR1 + CIITA | 0.778 | 0.671 | 0.884 |
| D 5/7 | CD177 + IL1R2 + CD74 + S100A9 + CX3CR1 + CIITA + CD3D | 0.771 | 0.665 | 0.877 |
| D 5/7 | CD177 + IL1R2 + CD74 + S100A9 + CX3CR1 + CIITA + CD3D + CTLA4 | 0.769 | 0.661 | 0.877 |
| D 5/7 | CD177 + IL1R2 + CD74 + S100A9 + CX3CR1 + CIITA + CTLA4 | 0.769 | 0.655 | 0.883 |
| D 5/7 | CD177 + IL1R2 + CD74 + S100A9 + CX3CR1 + CIITA + TAP2 | 0.787 | 0.688 | 0.886 |
| D 5/7 | CD177 + IL1R2 + CD74 + S100A9 + CX3CR1 + CIITA + TAP2 + CD3D | 0.781 | 0.682 | 0.88 |
| D 5/7 | CD177 + IL1R2 + CD74 + S100A9 + CX3CR1 + CIITA + TAP2 + CD3D + CTLA4 | 0.781 | 0.683 | 0.879 |
| D 5/7 | CD177 + IL1R2 + CD74 + S100A9 + CX3CR1 + CIITA + TAP2 + CTLA4 | 0.782 | 0.68 | 0.884 |
| D 5/7 | CD177 + IL1R2 + CD74 + S100A9 + CX3CR1 + CTLA4 | 0.767 | 0.665 | 0.869 |
| D 5/7 | CD177 + IL1R2 + CD74 + S100A9 + CX3CR1 + TAP2 | 0.779 | 0.684 | 0.874 |
| D 5/7 | CD177 + IL1R2 + CD74 + S100A9 + CX3CR1 + TAP2 + CD3D | 0.78 | 0.69 | 0.87 |
| D 5/7 | CD177 + IL1R2 + CD74 + S100A9 + CX3CR1 + TAP2 + CD3D + CTLA4 | 0.778 | 0.687 | 0.868 |
| D 5/7 | CD177 + IL1R2 + CD74 + S100A9 + CX3CR1 + TAP2 + CTLA4 | 0.779 | 0.683 | 0.874 |
| D 5/7 | CD177 + IL1R2 + CD74 + S100A9 + TAP2 | 0.775 | 0.679 | 0.871 |
| D 5/7 | CD177 + IL1R2 + CD74 + S100A9 + TAP2 + CD3D | 0.777 | 0.685 | 0.868 |
| D 5/7 | CD177 + IL1R2 + CD74 + S100A9 + TAP2 + CD3D + CTLA4 | 0.778 | 0.687 | 0.868 |
| D 5/7 | CD177 + IL1R2 + CD74 + S100A9 + TAP2 + CTLA4 | 0.776 | 0.68 | 0.872 |
| D 5/7 | CD177 + IL1R2 + CD74 + TAP2 | 0.768 | 0.662 | 0.874 |
| D 5/7 | CD177 + IL1R2 + CD74 + TAP2 + CD3D | 0.764 | 0.659 | 0.869 |
| D 5/7 | CD177 + IL1R2 + CD74 + TAP2 + CD3D + CTLA4 | 0.763 | 0.658 | 0.869 |
| D 5/7 | CD177 + IL1R2 + CD74 + TAP2 + CTLA4 | 0.767 | 0.66 | 0.875 |
| D 5/7 | CD177 + IL1R2 + CIITA | 0.72 | 0.605 | 0.836 |
| D 5/7 | CD177 + IL1R2 + CIITA + CD3D | 0.731 | 0.617 | 0.846 |
| D 5/7 | CD177 + IL1R2 + CIITA + CD3D + CTLA4 | 0.723 | 0.605 | 0.842 |
| D 5/7 | CD177 + IL1R2 + CIITA + CTLA4 | 0.715 | 0.592 | 0.838 |
| D 5/7 | CD177 + IL1R2 + CIITA + TAP2 | 0.763 | 0.66 | 0.867 |
| D 5/7 | CD177 + IL1R2 + CIITA + TAP2 + CD3D | 0.759 | 0.655 | 0.863 |
| D 5/7 | CD177 + IL1R2 + CIITA + TAP2 + CD3D + CTLA4 | 0.758 | 0.654 | 0.863 |
| D 5/7 | CD177 + IL1R2 + CIITA + TAP2 + CTLA4 | 0.761 | 0.655 | 0.867 |
| D 5/7 | CD177 + IL1R2 + CTLA4 | 0.704 | 0.57 | 0.839 |
| D 5/7 | CD177 + IL1R2 + CX3CR1 | 0.732 | 0.608 | 0.857 |
| D 5/7 | CD177 + IL1R2 + CX3CR1 + CD3D | 0.733 | 0.606 | 0.86 |
| D 5/7 | CD177 + IL1R2 + CX3CR1 + CD3D + CTLA4 | 0.726 | 0.593 | 0.859 |
| D 5/7 | CD177 + IL1R2 + CX3CR1 + CIITA | 0.734 | 0.619 | 0.849 |
| D 5/7 | CD177 + IL1R2 + CX3CR1 + CIITA + CD3D | 0.744 | 0.633 | 0.856 |
| D 5/7 | CD177 + IL1R2 + CX3CR1 + CIITA + CD3D + CTLA4 | 0.731 | 0.611 | 0.851 |
| D 5/7 | CD177 + IL1R2 + CX3CR1 + CIITA + CTLA4 | 0.729 | 0.606 | 0.852 |
| D 5/7 | CD177 + IL1R2 + CX3CR1 + CIITA + TAP2 | 0.763 | 0.659 | 0.866 |
| D 5/7 | CD177 + IL1R2 + CX3CR1 + CIITA + TAP2 + CD3D | 0.758 | 0.655 | 0.862 |
| D 5/7 | CD177 + IL1R2 + CX3CR1 + CIITA + TAP2 + CD3D + CTLA4 | 0.758 | 0.654 | 0.862 |
| D 5/7 | CD177 + IL1R2 + CX3CR1 + CIITA + TAP2 + CTLA4 | 0.761 | 0.655 | 0.866 |
| D 5/7 | CD177 + IL1R2 + CX3CR1 + CTLA4 | 0.719 | 0.582 | 0.856 |
| D 5/7 | CD177 + IL1R2 + CX3CR1 + TAP2 | 0.763 | 0.659 | 0.866 |
| D 5/7 | CD177 + IL1R2 + CX3CR1 + TAP2 + CD3D | 0.757 | 0.652 | 0.862 |
| D 5/7 | CD177 + IL1R2 + CX3CR1 + TAP2 + CD3D + CTLA4 | 0.758 | 0.654 | 0.862 |
| D 5/7 | CD177 + IL1R2 + CX3CR1 + TAP2 + CTLA4 | 0.76 | 0.654 | 0.866 |
| D 5/7 | CD177 + IL1R2 + IFNG | 0.702 | 0.568 | 0.836 |
| D 5/7 | CD177 + IL1R2 + IFNG + CD3D | 0.71 | 0.575 | 0.846 |
| D 5/7 | CD177 + IL1R2 + IFNG + CD3D + CTLA4 | 0.709 | 0.576 | 0.843 |
| D 5/7 | CD177 + IL1R2 + IFNG + CIITA | 0.726 | 0.611 | 0.842 |
| D 5/7 | CD177 + IL1R2 + IFNG + CIITA + CD3D | 0.735 | 0.615 | 0.855 |
| D 5/7 | CD177 + IL1R2 + IFNG + CIITA + CD3D + CTLA4 | 0.732 | 0.61 | 0.853 |
| D 5/7 | CD177 + IL1R2 + IFNG + CIITA + CTLA4 | 0.72 | 0.599 | 0.841 |
| D 5/7 | CD177 + IL1R2 + IFNG + CIITA + TAP2 | 0.771 | 0.665 | 0.877 |
| D 5/7 | CD177 + IL1R2 + IFNG + CIITA + TAP2 + CD3D | 0.768 | 0.661 | 0.876 |
| D 5/7 | CD177 + IL1R2 + IFNG + CIITA + TAP2 + CD3D + CTLA4 | 0.769 | 0.663 | 0.875 |
| D 5/7 | CD177 + IL1R2 + IFNG + CIITA + TAP2 + CTLA4 | 0.773 | 0.667 | 0.879 |
| D 5/7 | CD177 + IL1R2 + IFNG + CTLA4 | 0.698 | 0.56 | 0.836 |
| D 5/7 | CD177 + IL1R2 + IFNG + CX3CR1 | 0.733 | 0.609 | 0.858 |
| D 5/7 | CD177 + IL1R2 + IFNG + CX3CR1 + CD3D | 0.726 | 0.592 | 0.86 |
| D 5/7 | CD177 + IL1R2 + IFNG + CX3CR1 + CD3D + CTLA4 | 0.713 | 0.579 | 0.848 |
| D 5/7 | CD177 + IL1R2 + IFNG + CX3CR1 + CIITA | 0.752 | 0.646 | 0.859 |
| D 5/7 | CD177 + IL1R2 + IFNG + CX3CR1 + CIITA + CD3D | 0.754 | 0.635 | 0.873 |
| D 5/7 | CD177 + IL1R2 + IFNG + CX3CR1 + CIITA + CD3D + CTLA4 | 0.751 | 0.633 | 0.869 |
| D 5/7 | CD177 + IL1R2 + IFNG + CX3CR1 + CIITA + CTLA4 | 0.739 | 0.621 | 0.858 |
| D 5/7 | CD177 + IL1R2 + IFNG + CX3CR1 + CIITA + TAP2 | 0.778 | 0.674 | 0.882 |
| D 5/7 | CD177 + IL1R2 + IFNG + CX3CR1 + CIITA + TAP2 + CD3D | 0.775 | 0.671 | 0.88 |
| D 5/7 | CD177 + IL1R2 + IFNG + CX3CR1 + CIITA + TAP2 + CD3D + CTLA4 | 0.777 | 0.673 | 0.88 |
| D 5/7 | CD177 + IL1R2 + IFNG + CX3CR1 + CIITA + TAP2 + CTLA4 | 0.774 | 0.669 | 0.88 |
| D 5/7 | CD177 + IL1R2 + IFNG + CX3CR1 + CTLA4 | 0.714 | 0.581 | 0.847 |
| D 5/7 | CD177 + IL1R2 + IFNG + CX3CR1 + TAP2 | 0.768 | 0.658 | 0.877 |
| D 5/7 | CD177 + IL1R2 + IFNG + CX3CR1 + TAP2 + CD3D | 0.763 | 0.648 | 0.877 |
| D 5/7 | CD177 + IL1R2 + IFNG + CX3CR1 + TAP2 + CD3D + CTLA4 | 0.763 | 0.65 | 0.877 |
| D 5/7 | CD177 + IL1R2 + IFNG + CX3CR1 + TAP2 + CTLA4 | 0.767 | 0.656 | 0.878 |
| D 5/7 | CD177 + IL1R2 + IFNG + S100A9 | 0.747 | 0.632 | 0.862 |
| D 5/7 | CD177 + IL1R2 + IFNG + S100A9 + CD3D | 0.762 | 0.641 | 0.883 |
| D 5/7 | CD177 + IL1R2 + IFNG + S100A9 + CD3D + CTLA4 | 0.762 | 0.641 | 0.883 |
| D 5/7 | CD177 + IL1R2 + IFNG + S100A9 + CIITA | 0.761 | 0.656 | 0.866 |
| D 5/7 | CD177 + IL1R2 + IFNG + S100A9 + CIITA + CD3D | 0.765 | 0.651 | 0.879 |
| D 5/7 | CD177 + IL1R2 + IFNG + S100A9 + CIITA + CD3D + CTLA4 | 0.766 | 0.652 | 0.88 |
| D 5/7 | CD177 + IL1R2 + IFNG + S100A9 + CIITA + CTLA4 | 0.748 | 0.629 | 0.866 |
| D 5/7 | CD177 + IL1R2 + IFNG + S100A9 + CIITA + TAP2 | 0.793 | 0.699 | 0.888 |
| D 5/7 | CD177 + IL1R2 + IFNG + S100A9 + CIITA + TAP2 + CD3D | 0.798 | 0.7 | 0.896 |
| D 5/7 | CD177 + IL1R2 + IFNG + S100A9 + CIITA + TAP2 + CD3D + CTLA4 | 0.798 | 0.701 | 0.894 |
| D 5/7 | CD177 + IL1R2 + IFNG + S100A9 + CIITA + TAP2 + CTLA4 | 0.795 | 0.7 | 0.889 |
| D 5/7 | CD177 + IL1R2 + IFNG + S100A9 + CTLA4 | 0.737 | 0.609 | 0.865 |
| D 5/7 | CD177 + IL1R2 + IFNG + S100A9 + CX3CR1 | 0.787 | 0.677 | 0.897 |
| D 5/7 | CD177 + IL1R2 + IFNG + S100A9 + CX3CR1 + CD3D | 0.778 | 0.657 | 0.9 |
| D 5/7 | CD177 + IL1R2 + IFNG + S100A9 + CX3CR1 + CD3D + CTLA4 | 0.779 | 0.654 | 0.903 |
| D 5/7 | CD177 + IL1R2 + IFNG + S100A9 + CX3CR1 + CIITA | 0.797 | 0.698 | 0.895 |
| D 5/7 | CD177 + IL1R2 + IFNG + S100A9 + CX3CR1 + CIITA + CD3D | 0.789 | 0.678 | 0.9 |
| D 5/7 | CD177 + IL1R2 + IFNG + S100A9 + CX3CR1 + CIITA + CD3D + CTLA4 | 0.788 | 0.675 | 0.901 |
| D 5/7 | CD177 + IL1R2 + IFNG + S100A9 + CX3CR1 + CIITA + CTLA4 | 0.785 | 0.673 | 0.898 |
| D 5/7 | CD177 + IL1R2 + IFNG + S100A9 + CX3CR1 + CIITA + TAP2 | 0.809 | 0.715 | 0.904 |
| D 5/7 | CD177 + IL1R2 + IFNG + S100A9 + CX3CR1 + CIITA + TAP2 + CD3D | 0.806 | 0.706 | 0.906 |
| D 5/7 | CD177 + IL1R2 + IFNG + S100A9 + CX3CR1 + CIITA + TAP2 + CD3D + CTLA4 | 0.807 | 0.709 | 0.905 |
| D 5/7 | CD177 + IL1R2 + IFNG + S100A9 + CX3CR1 + CIITA + TAP2 + CTLA4 | 0.808 | 0.712 | 0.903 |
| D 5/7 | CD177 + IL1R2 + IFNG + S100A9 + CX3CR1 + CTLA4 | 0.775 | 0.655 | 0.896 |
| D 5/7 | CD177 + IL1R2 + IFNG + S100A9 + CX3CR1 + TAP2 | 0.809 | 0.714 | 0.904 |
| D 5/7 | CD177 + IL1R2 + IFNG + S100A9 + CX3CR1 + TAP2 + CD3D | 0.802 | 0.698 | 0.907 |
| D 5/7 | CD177 + IL1R2 + IFNG + S100A9 + CX3CR1 + TAP2 + CD3D + CTLA4 | 0.805 | 0.706 | 0.904 |
| D 5/7 | CD177 + IL1R2 + IFNG + S100A9 + CX3CR1 + TAP2 + CTLA4 | 0.807 | 0.71 | 0.903 |
| D 5/7 | CD177 + IL1R2 + IFNG + S100A9 + TAP2 | 0.795 | 0.701 | 0.889 |
| D 5/7 | CD177 + IL1R2 + IFNG + S100A9 + TAP2 + CD3D | 0.798 | 0.699 | 0.897 |
| D 5/7 | CD177 + IL1R2 + IFNG + S100A9 + TAP2 + CD3D + CTLA4 | 0.803 | 0.708 | 0.899 |
| D 5/7 | CD177 + IL1R2 + IFNG + S100A9 + TAP2 + CTLA4 | 0.795 | 0.702 | 0.889 |
| D 5/7 | CD177 + IL1R2 + IFNG + TAP2 | 0.765 | 0.655 | 0.875 |
| D 5/7 | CD177 + IL1R2 + IFNG + TAP2 + CD3D | 0.762 | 0.648 | 0.875 |
| D 5/7 | CD177 + IL1R2 + IFNG + TAP2 + CD3D + CTLA4 | 0.765 | 0.653 | 0.877 |
| D 5/7 | CD177 + IL1R2 + IFNG + TAP2 + CTLA4 | 0.763 | 0.653 | 0.874 |
| D 5/7 | CD177 + IL1R2 + S100A9 | 0.747 | 0.641 | 0.853 |
| D 5/7 | CD177 + IL1R2 + S100A9 + CD3D | 0.748 | 0.629 | 0.867 |
| D 5/7 | CD177 + IL1R2 + S100A9 + CD3D + CTLA4 | 0.742 | 0.62 | 0.864 |
| D 5/7 | CD177 + IL1R2 + S100A9 + CIITA | 0.751 | 0.648 | 0.853 |
| D 5/7 | CD177 + IL1R2 + S100A9 + CIITA + CD3D | 0.75 | 0.634 | 0.866 |
| D 5/7 | CD177 + IL1R2 + S100A9 + CIITA + CD3D + CTLA4 | 0.742 | 0.623 | 0.86 |
| D 5/7 | CD177 + IL1R2 + S100A9 + CIITA + CTLA4 | 0.73 | 0.608 | 0.852 |
| D 5/7 | CD177 + IL1R2 + S100A9 + CIITA + TAP2 | 0.778 | 0.683 | 0.873 |
| D 5/7 | CD177 + IL1R2 + S100A9 + CIITA + TAP2 + CD3D | 0.77 | 0.671 | 0.869 |
| D 5/7 | CD177 + IL1R2 + S100A9 + CIITA + TAP2 + CD3D + CTLA4 | 0.776 | 0.68 | 0.872 |
| D 5/7 | CD177 + IL1R2 + S100A9 + CIITA + TAP2 + CTLA4 | 0.777 | 0.682 | 0.872 |
| D 5/7 | CD177 + IL1R2 + S100A9 + CTLA4 | 0.732 | 0.61 | 0.854 |
| D 5/7 | CD177 + IL1R2 + S100A9 + CX3CR1 | 0.764 | 0.652 | 0.876 |
| D 5/7 | CD177 + IL1R2 + S100A9 + CX3CR1 + CD3D | 0.763 | 0.647 | 0.879 |
| D 5/7 | CD177 + IL1R2 + S100A9 + CX3CR1 + CD3D + CTLA4 | 0.754 | 0.635 | 0.873 |
| D 5/7 | CD177 + IL1R2 + S100A9 + CX3CR1 + CIITA | 0.763 | 0.655 | 0.872 |
| D 5/7 | CD177 + IL1R2 + S100A9 + CX3CR1 + CIITA + CD3D | 0.762 | 0.648 | 0.875 |
| D 5/7 | CD177 + IL1R2 + S100A9 + CX3CR1 + CIITA + CD3D + CTLA4 | 0.752 | 0.634 | 0.87 |
| D 5/7 | CD177 + IL1R2 + S100A9 + CX3CR1 + CIITA + CTLA4 | 0.755 | 0.633 | 0.876 |
| D 5/7 | CD177 + IL1R2 + S100A9 + CX3CR1 + CIITA + TAP2 | 0.784 | 0.688 | 0.88 |
| D 5/7 | CD177 + IL1R2 + S100A9 + CX3CR1 + CIITA + TAP2 + CD3D | 0.772 | 0.673 | 0.871 |
| D 5/7 | CD177 + IL1R2 + S100A9 + CX3CR1 + CIITA + TAP2 + CD3D + CTLA4 | 0.777 | 0.681 | 0.874 |
| D 5/7 | CD177 + IL1R2 + S100A9 + CX3CR1 + CIITA + TAP2 + CTLA4 | 0.78 | 0.683 | 0.877 |
| D 5/7 | CD177 + IL1R2 + S100A9 + CX3CR1 + CTLA4 | 0.755 | 0.632 | 0.877 |
| D 5/7 | CD177 + IL1R2 + S100A9 + CX3CR1 + TAP2 | 0.777 | 0.683 | 0.871 |
| D 5/7 | CD177 + IL1R2 + S100A9 + CX3CR1 + TAP2 + CD3D | 0.774 | 0.68 | 0.868 |
| D 5/7 | CD177 + IL1R2 + S100A9 + CX3CR1 + TAP2 + CD3D + CTLA4 | 0.775 | 0.682 | 0.868 |
| D 5/7 | CD177 + IL1R2 + S100A9 + CX3CR1 + TAP2 + CTLA4 | 0.778 | 0.683 | 0.872 |
| D 5/7 | CD177 + IL1R2 + S100A9 + TAP2 | 0.779 | 0.686 | 0.872 |
| D 5/7 | CD177 + IL1R2 + S100A9 + TAP2 + CD3D | 0.773 | 0.678 | 0.867 |
| D 5/7 | CD177 + IL1R2 + S100A9 + TAP2 + CD3D + CTLA4 | 0.774 | 0.68 | 0.867 |
| D 5/7 | CD177 + IL1R2 + S100A9 + TAP2 + CTLA4 | 0.778 | 0.684 | 0.872 |
| D 5/7 | CD177 + IL1R2 + TAP2 | 0.763 | 0.66 | 0.867 |
| D 5/7 | CD177 + IL1R2 + TAP2 + CD3D | 0.757 | 0.651 | 0.864 |
| D 5/7 | CD177 + IL1R2 + TAP2 + CD3D + CTLA4 | 0.757 | 0.65 | 0.863 |
| D 5/7 | CD177 + IL1R2 + TAP2 + CTLA4 | 0.761 | 0.656 | 0.867 |
| D 5/7 | CD177 + S100A9 + CD3D | 0.751 | 0.638 | 0.864 |
| D 5/7 | CD177 + S100A9 + CD3D + CTLA4 | 0.745 | 0.629 | 0.861 |
| D 5/7 | CD177 + S100A9 + CIITA | 0.73 | 0.636 | 0.823 |
| D 5/7 | CD177 + S100A9 + CIITA + CD3D | 0.758 | 0.65 | 0.866 |
| D 5/7 | CD177 + S100A9 + CIITA + CD3D + CTLA4 | 0.75 | 0.638 | 0.861 |
| D 5/7 | CD177 + S100A9 + CIITA + CTLA4 | 0.725 | 0.609 | 0.841 |
| D 5/7 | CD177 + S100A9 + CIITA + TAP2 | 0.78 | 0.694 | 0.865 |
| D 5/7 | CD177 + S100A9 + CIITA + TAP2 + CD3D | 0.778 | 0.685 | 0.871 |
| D 5/7 | CD177 + S100A9 + CIITA + TAP2 + CD3D + CTLA4 | 0.778 | 0.686 | 0.87 |
| D 5/7 | CD177 + S100A9 + CIITA + TAP2 + CTLA4 | 0.781 | 0.693 | 0.87 |
| D 5/7 | CD177 + S100A9 + CTLA4 | 0.722 | 0.602 | 0.842 |
| D 5/7 | CD177 + S100A9 + CX3CR1 | 0.77 | 0.663 | 0.878 |
| D 5/7 | CD177 + S100A9 + CX3CR1 + CD3D | 0.772 | 0.66 | 0.883 |
| D 5/7 | CD177 + S100A9 + CX3CR1 + CD3D + CTLA4 | 0.766 | 0.65 | 0.883 |
| D 5/7 | CD177 + S100A9 + CX3CR1 + CIITA | 0.767 | 0.662 | 0.871 |
| D 5/7 | CD177 + S100A9 + CX3CR1 + CIITA + CD3D | 0.778 | 0.669 | 0.886 |
| D 5/7 | CD177 + S100A9 + CX3CR1 + CIITA + CD3D + CTLA4 | 0.768 | 0.655 | 0.882 |
| D 5/7 | CD177 + S100A9 + CX3CR1 + CIITA + CTLA4 | 0.768 | 0.653 | 0.883 |
| D 5/7 | CD177 + S100A9 + CX3CR1 + CIITA + TAP2 | 0.788 | 0.696 | 0.88 |
| D 5/7 | CD177 + S100A9 + CX3CR1 + CIITA + TAP2 + CD3D | 0.787 | 0.692 | 0.882 |
| D 5/7 | CD177 + S100A9 + CX3CR1 + CIITA + TAP2 + CD3D + CTLA4 | 0.788 | 0.693 | 0.884 |
| D 5/7 | CD177 + S100A9 + CX3CR1 + CIITA + TAP2 + CTLA4 | 0.786 | 0.689 | 0.884 |
| D 5/7 | CD177 + S100A9 + CX3CR1 + CTLA4 | 0.767 | 0.65 | 0.883 |
| D 5/7 | CD177 + S100A9 + CX3CR1 + TAP2 | 0.787 | 0.697 | 0.876 |
| D 5/7 | CD177 + S100A9 + CX3CR1 + TAP2 + CD3D | 0.789 | 0.695 | 0.883 |
| D 5/7 | CD177 + S100A9 + CX3CR1 + TAP2 + CD3D + CTLA4 | 0.788 | 0.694 | 0.883 |
| D 5/7 | CD177 + S100A9 + CX3CR1 + TAP2 + CTLA4 | 0.781 | 0.685 | 0.878 |
| D 5/7 | CD177 + S100A9 + TAP2 | 0.779 | 0.694 | 0.864 |
| D 5/7 | CD177 + S100A9 + TAP2 + CD3D | 0.779 | 0.687 | 0.87 |
| D 5/7 | CD177 + S100A9 + TAP2 + CD3D + CTLA4 | 0.778 | 0.686 | 0.87 |
| D 5/7 | CD177 + S100A9 + TAP2 + CTLA4 | 0.777 | 0.688 | 0.867 |
| D 5/7 | CD177 + TAP2 + CD3D | 0.752 | 0.654 | 0.851 |
| D 5/7 | CD177 + TAP2 + CD3D + CTLA4 | 0.75 | 0.65 | 0.851 |
| D 5/7 | CD177 + TAP2 + CTLA4 | 0.761 | 0.661 | 0.862 |

Table 6 Performance (AUC and 95% confidence interval, with AUC2.5 and AUC 97.5 limits) of the measurement of the expression of CD177, in combination with one or more other biomarker(s) (multivariate analysis) measured on D3/4 or D5/7 from inclusion in the cohort, in predicting the incidence of a healthcare-associated infection before D15 from inclusion in the cohort in patients in a septic state/in septic shock, suffering from severe injuries, or hospitalized following major surgery.

TABLE 7

| Maximum time interval | | | | |
|---|---|---|---|---|
| between the sample being | | | | |
| taken and the potential | | | | |
| incidence of first | | | | |
| healthcare-associated | | | | |
| infection | Biomarkers | AUC | AUC · 2.5 | AUC · 97.5 |
| 4 days | CD177 + CD3D | 0.688 | 0.497 | 0.878 |
| 4 days | CD177 + NFkB1 | 0.688 | 0.497 | 0.878 |
| 4 days | CD177 + ALOX5 | 0.691 | 0.502 | 0.881 |
| 4 days | CD177 + IFNG | 0.691 | 0.5 | 0.883 |
| 4 days | CD177 + IL1RN | 0.691 | 0.498 | 0.884 |
| 4 days | CD177 + IP10 | 0.691 | 0.501 | 0.882 |
| 4 days | CD177 + RORC | 0.691 | 0.496 | 0.887 |
| 4 days | CD177 + TAP2 | 0.691 | 0.495 | 0.888 |
| 4 days | CD177 + ADGRE3 | 0.695 | 0.507 | 0.884 |
| 4 days | CD177 + GATA3 | 0.695 | 0.502 | 0.889 |
| 4 days | CD177 + ARL14EP | 0.699 | 0.515 | 0.884 |
| 4 days | CD177 + CX3CR1 | 0.699 | 0.511 | 0.887 |
| 4 days | CD177 + OAS2 | 0.699 | 0.503 | 0.895 |
| 4 days | CD177 + TNF | 0.699 | 0.505 | 0.893 |
| 4 days | CD177 + IL6 | 0.702 | 0.503 | 0.902 |
| 4 days | CD177 + HAVCR2 | 0.703 | 0.517 | 0.89 |
| 4 days | CD177 + FCGR1A | 0.707 | 0.516 | 0.898 |
| 4 days | CD177 + CIITA | 0.707 | 0.521 | 0.893 |
| 4 days | CD177 + MDC1 | 0.711 | 0.521 | 0.901 |
| 4 days | CD177 + CCNB1IP1 | 0.715 | 0.53 | 0.9 |
| 4 days | CD177 + TDRD9 | 0.715 | 0.526 | 0.904 |
| 4 days | CD177 + IL10 | 0.723 | 0.542 | 0.903 |
| 4 days | CD177 + GNLY | 0.734 | 0.559 | 0.91 |
| 4 days | CD177 + TBX21 | 0.734 | 0.555 | 0.914 |
| 4 days | CD177 + LILRB2 | 0.738 | 0.564 | 0.913 |
| 4 days | CD177 + S100A9 | 0.746 | 0.561 | 0.931 |
| 4 days | CD177 + IL18 | 0.75 | 0.565 | 0.935 |
| 4 days | CD177 + CD74 | 0.762 | 0.585 | 0.938 |
| 4 days | CD177 + C3AR1 + CD74 | 0.707 | 0.521 | 0.893 |
| 4 days | CD177 + C3AR1 + CD74 + CD3D | 0.719 | 0.536 | 0.902 |
| 4 days | CD177 + C3AR1 + CD74 + CD3D + CTLA4 | 0.73 | 0.551 | 0.91 |
| 4 days | CD177 + C3AR1 + CD74 + CIITA | 0.723 | 0.539 | 0.906 |
| 4 days | CD177 + C3AR1 + CD74 + CIITA + CD3D | 0.723 | 0.542 | 0.904 |
| 4 days | CD177 + C3AR1 + CD74 + CIITA + CD3D + CTLA4 | 0.727 | 0.546 | 0.907 |
| 4 days | CD177 + C3AR1 + CD74 + CIITA + CTLA4 | 0.727 | 0.546 | 0.907 |
| 4 days | CD177 + C3AR1 + CD74 + CIITA + TAP2 | 0.719 | 0.534 | 0.903 |
| 4 days | CD177 + C3AR1 + CD74 + CIITA + TAP2 + CD3D | 0.723 | 0.541 | 0.904 |
| 4 days | CD177 + C3AR1 + CD74 + CIITA + TAP2 + CD3D + CTLA4 | 0.742 | 0.564 | 0.92 |
| 4 days | CD177 + C3AR1 + CD74 + CIITA + TAP2 + CTLA4 | 0.738 | 0.558 | 0.919 |
| 4 days | CD177 + C3AR1 + CD74 + CTLA4 | 0.73 | 0.551 | 0.91 |
| 4 days | CD177 + C3AR1 + CD74 + CX3CR1 | 0.734 | 0.553 | 0.915 |
| 4 days | CD177 + C3AR1 + CD74 + CX3CR1 + CD3D | 0.742 | 0.567 | 0.917 |
| 4 days | CD177 + C3AR1 + CD74 + CX3CR1 + CD3D + CTLA4 | 0.75 | 0.576 | 0.924 |
| 4 days | CD177 + C3AR1 + CD74 + CX3CR1 + CIITA | 0.762 | 0.588 | 0.936 |
| 4 days | CD177 + C3AR1 + CD74 + CX3CR1 + CIITA + CD3D | 0.762 | 0.592 | 0.931 |
| 4 days | CD177 + C3AR1 + CD74 + CX3CR1 + CIITA + CD3D + CTLA4 | 0.758 | 0.586 | 0.929 |
| 4 days | CD177 + C3AR1 + CD74 + CX3CR1 + CIITA + CTLA4 | 0.754 | 0.579 | 0.929 |
| 4 days | CD177 + C3AR1 + CD74 + CX3CR1 + CIITA + TAP2 | 0.77 | 0.603 | 0.936 |
| 4 days | CD177 + C3AR1 + CD74 + CX3CR1 + CIITA + TAP2 + CD3D | 0.781 | 0.617 | 0.945 |
| 4 days | CD177 + C3AR1 + CD74 + CX3CR1 + CIITA + TAP2 + CD3D + | 0.785 | 0.622 | 0.948 |
| | CTLA4 | | | |
| 4 days | CD177 + C3AR1 + CD74 + CX3CR1 + CIITA + TAP2 + CTLA4 | 0.77 | 0.601 | 0.938 |
| 4 days | CD177 + C3AR1 + CD74 + CX3CR1 + CTLA4 | 0.754 | 0.576 | 0.932 |
| 4 days | CD177 + C3AR1 + CD74 + CX3CR1 + TAP2 | 0.777 | 0.612 | 0.943 |
| 4 days | CD177 + C3AR1 + CD74 + CX3CR1 + TAP2 + CD3D | 0.777 | 0.61 | 0.945 |
| 4 days | CD177 + C3AR1 + CD74 + CX3CR1 + TAP2 + CD3D + CTLA4 | 0.809 | 0.649 | 0.968 |
| 4 days | CD177 + C3AR1 + CD74 + CX3CR1 + TAP2 + CTLA4 | 0.805 | 0.646 | 0.963 |
| 4 days | CD177 + C3AR1 + CD74 + IFNG | 0.727 | 0.548 | 0.905 |
| 4 days | CD177 + C3AR1 + CD74 + IFNG + CD3D | 0.727 | 0.545 | 0.909 |
| 4 days | CD177 + C3AR1 + CD74 + IFNG + CD3D + CTLA4 | 0.723 | 0.541 | 0.905 |
| 4 days | CD177 + C3AR1 + CD74 + IFNG + CIITA | 0.727 | 0.548 | 0.905 |
| 4 days | CD177 + C3AR1 + CD74 + IFNG + CIITA + CD3D | 0.727 | 0.545 | 0.909 |
| 4 days | CD177 + C3AR1 + CD74 + IFNG + CIITA + CD3D + CTLA4 | 0.715 | 0.527 | 0.903 |
| 4 days | CD177 + C3AR1 + CD74 + IFNG + CIITA + CTLA4 | 0.723 | 0.537 | 0.909 |
| 4 days | CD177 + C3AR1 + CD74 + IFNG + CIITA + TAP2 | 0.723 | 0.543 | 0.902 |
| 4 days | CD177 + C3AR1 + CD74 + IFNG + CIITA + TAP2 + CD3D | 0.707 | 0.523 | 0.891 |
| 4 days | CD177 + C3AR1 + CD74 + IFNG + CIITA + TAP2 + CD3D + | 0.719 | 0.532 | 0.905 |
| | CTLA4 | | | |
| 4 days | CD177 + C3AR1 + CD74 + IFNG + CIITA + TAP2 + CTLA4 | 0.73 | 0.548 | 0.913 |
| 4 days | CD177 + C3AR1 + CD74 + IFNG + CTLA4 | 0.723 | 0.538 | 0.907 |
| 4 days | CD177 + C3AR1 + CD74 + IFNG + CX3CR1 | 0.855 | 0.714 | 0.997 |
| 4 days | CD177 + C3AR1 + CD74 + IFNG + CX3CR1 + CD3D | 0.871 | 0.748 | 0.994 |
| 4 days | CD177 + C3AR1 + CD74 + IFNG + CX3CR1 + CD3D + CTLA4 | 0.891 | 0.778 | 1 |
| 4 days | CD177 + C3AR1 + CD74 + IFNG + CX3CR1 + CIITA | 0.879 | 0.754 | 1 |
| 4 days | CD177 + C3AR1 + CD74 + IFNG + CX3CR1 + CIITA + CD3D | 0.887 | 0.776 | 0.998 |
| 4 days | CD177 + C3AR1 + CD74 + IFNG + CX3CR1 + CIITA + CD3D + | 0.895 | 0.784 | 1 |
| | CTLA4 | | | |
| 4 days | CD177 + C3AR1 + CD74 + IFNG + CX3CR1 + CIITA + CTLA4 | 0.895 | 0.784 | 1 |
| 4 days | CD177 + C3AR1 + CD74 + IFNG + CX3CR1 + CIITA + TAP2 | 0.871 | 0.744 | 0.998 |
| 4 days | CD177 + C3AR1 + CD74 + IFNG + CX3CR1 + CIITA + TAP2 + | 0.875 | 0.758 | 0.992 |
| | CD3D | | | |
| 4 days | CD177 + C3AR1 + CD74 + IFNG + CX3CR1 + CIITA + TAP2 + | 0.867 | 0.74 | 0.994 |
| | CD3D + CTLA4 | | | |
| 4 days | CD177 + C3AR1 + CD74 + IFNG + CX3CR1 + CIITA + TAP2 + | 0.867 | 0.742 | 0.992 |
| | CTLA4 | | | |
| 4 days | CD177 + C3AR1 + CD74 + IFNG + CX3CR1 + CTLA4 | 0.898 | 0.79 | 1 |
| 4 days | CD177 + C3AR1 + CD74 + IFNG + CX3CR1 + TAP2 | 0.852 | 0.712 | 0.992 |
| 4 days | CD177 + C3AR1 + CD74 + IFNG + CX3CR1 + TAP2 + CD3D | 0.883 | 0.766 | 0.999 |
| 4 days | CD177 + C3AR1 + CD74 + IFNG + CX3CR1 + TAP2 + CD3D + | 0.867 | 0.74 | 0.994 |
| | CTLA4 | | | |
| 4 days | CD177 + C3AR1 + CD74 + IFNG + CX3CR1 + TAP2 + CTLA4 | 0.871 | 0.748 | 0.995 |
| 4 days | CD177 + C3AR1 + CD74 + IFNG + S100A9 | 0.789 | 0.624 | 0.954 |
| 4 days | CD177 + C3AR1 + CD74 + IFNG + S100A9 + CD3D | 0.785 | 0.62 | 0.951 |
| 4 days | CD177 + C3AR1 + CD74 + IFNG + S100A9 + CD3D + CTLA4 | 0.785 | 0.62 | 0.95 |
| 4 days | CD177 + C3AR1 + CD74 + IFNG + S100A9 + CIITA | 0.793 | 0.625 | 0.961 |
| 4 days | CD177 + C3AR1 + CD74 + IFNG + S100A9 + CIITA + CD3D | 0.793 | 0.628 | 0.958 |
| 4 days | CD177 + C3AR1 + CD74 + IFNG + S100A9 + CIITA + CD3D + | 0.797 | 0.633 | 0.961 |
| | CTLA4 | | | |
| 4 days | CD177 + C3AR1 + CD74 + IFNG + S100A9 + CIITA + CTLA4 | 0.785 | 0.617 | 0.953 |
| 4 days | CD177 + C3AR1 + CD74 + IFNG + S100A9 + CIITA + TAP2 | 0.801 | 0.628 | 0.974 |
| 4 days | CD177 + C3AR1 + CD74 + IFNG + S100A9 + CIITA + TAP2 + | 0.801 | 0.636 | 0.966 |
| | CD3D | | | |
| 4 days | CD177 + C3AR1 + CD74 + IFNG + S100A9 + CIITA + TAP2 + | 0.809 | 0.647 | 0.97 |
| | CD3D + CTLA4 | | | |
| 4 days | CD177 + C3AR1 + CD74 + IFNG + S100A9 + CIITA + TAP2 + | 0.797 | 0.631 | 0.963 |
| | CTLA4 | | | |
| 4 days | CD177 + C3AR1 + CD74 + IFNG + S100A9 + CTLA4 | 0.773 | 0.605 | 0.942 |
| 4 days | CD177 + C3AR1 + CD74 + IFNG + S100A9 + CX3CR1 | 0.938 | 0.851 | 1 |
| 4 days | CD177 + C3AR1 + CD74 + IFNG + S100A9 + CX3CR1 + CD3D | 0.953 | 0.891 | 1 |
| 4 days | CD177 + C3AR1 + CD74 + IFNG + S100A9 + CX3CR1 + CD3D + | 0.953 | 0.891 | 1 |
| | CTLA4 | | | |
| 4 days | CD177 + C3AR1 + CD74 + IFNG + S100A9 + CX3CR1 + CIITA | 0.941 | 0.857 | 1 |
| 4 days | CD177 + C3AR1 + CD74 + IFNG + S100A9 + CX3CR1 + CIITA + | 0.953 | 0.891 | 1 |
| | CD3D | | | |
| 4 days | CD177 + C3AR1 + CD74 + IFNG + S100A9 + CX3CR1 + CIITA + | 0.953 | 0.891 | 1 |
| | CD3D + CTLA4 | | | |
| 4 days | CD177 + C3AR1 + CD74 + IFNG + S100A9 + CX3CR1 + CIITA + | 0.961 | 0.905 | 1 |
| | CTLA4 | | | |
| 4 days | CD177 + C3AR1 + CD74 + IFNG + S100A9 + CX3CR1 + CIITA + | 0.934 | 0.845 | 1 |
| | TAP2 | | | |
| 4 days | CD177 + C3AR1 + CD74 + IFNG + S100A9 + CX3CR1 + CIITA + | 0.957 | 0.897 | 1 |
| | TAP2 + CD3D | | | |
| 4 days | CD177 + C3AR1 + CD74 + IFNG + S100A9 + CX3CR1 + CIITA + | 0.965 | 0.914 | 1 |
| | TAP2 + CD3D + CTLA4 | | | |
| 4 days | CD177 + C3AR1 + CD74 + IFNG + S100A9 + CX3CR1 + CIITA + | 0.953 | 0.889 | 1 |
| | TAP2 + CTLA4 | | | |
| 4 days | CD177 + C3AR1 + CD74 + IFNG + S100A9 + CX3CR1 + CTLA4 | 0.945 | 0.871 | 1 |
| 4 days | CD177 + C3AR1 + CD74 + IFNG + S100A9 + CX3CR1 + TAP2 | 0.934 | 0.845 | 1 |
| 4 days | CD177 + C3AR1 + CD74 + IFNG + S100A9 + CX3CR1 + TAP2 + | 0.957 | 0.897 | 1 |
| | CD3D | | | |
| 4 days | CD177 + C3AR1 + CD74 + IFNG + S100A9 + CX3CR1 + TAP2 + | 0.965 | 0.914 | 1 |
| | CD3D + CTLA4 | | | |
| 4 days | CD177 + C3AR1 + CD74 + IFNG + S100A9 + CX3CR1 + TAP2 + | 0.938 | 0.849 | 1 |
| | CTLA4 | | | |
| 4 days | CD177 + C3AR1 + CD74 + IFNG + S100A9 + TAP2 | 0.801 | 0.635 | 0.967 |
| 4 days | CD177 + C3AR1 + CD74 + IFNG + S100A9 + TAP2 + CD3D | 0.805 | 0.647 | 0.963 |
| 4 days | CD177 + C3AR1 + CD74 + IFNG + S100A9 + TAP2 + CD3D + | 0.805 | 0.648 | 0.961 |
| | CTLA4 | | | |
| 4 days | CD177 + C3AR1 + CD74 + IFNG + S100A9 + TAP2 + CTLA4 | 0.789 | 0.626 | 0.952 |
| 4 days | CD177 + C3AR1 + CD74 + IFNG + TAP2 | 0.719 | 0.538 | 0.899 |
| 4 days | CD177 + C3AR1 + CD74 + IFNG + TAP2 + CD3D | 0.715 | 0.532 | 0.898 |
| 4 days | CD177 + C3AR1 + CD74 + IFNG + TAP2 + CD3D + CTLA4 | 0.719 | 0.536 | 0.901 |
| 4 days | CD177 + C3AR1 + CD74 + IFNG + TAP2 + CTLA4 | 0.715 | 0.53 | 0.9 |
| 4 days | CD177 + C3AR1 + CD74 + S100A9 | 0.777 | 0.61 | 0.945 |
| 4 days | CD177 + C3AR1 + CD74 + S100A9 + CD3D | 0.785 | 0.622 | 0.948 |
| 4 days | CD177 + C3AR1 + CD74 + S100A9 + CD3D + CTLA4 | 0.781 | 0.615 | 0.947 |
| 4 days | CD177 + C3AR1 + CD74 + S100A9 + CIITA | 0.801 | 0.634 | 0.968 |
| 4 days | CD177 + C3AR1 + CD74 + S100A9 + CIITA + CD3D | 0.789 | 0.622 | 0.956 |
| 4 days | CD177 + C3AR1 + CD74 + S100A9 + CIITA + CD3D + CTLA4 | 0.805 | 0.644 | 0.965 |
| 4 days | CD177 + C3AR1 + CD74 + S100A9 + CIITA + CTLA4 | 0.797 | 0.632 | 0.962 |
| 4 days | CD177 + C3AR1 + CD74 + S100A9 + CIITA + TAP2 | 0.809 | 0.638 | 0.98 |
| 4 days | CD177 + C3AR1 + CD74 + S100A9 + CIITA + TAP2 + CD3D | 0.809 | 0.644 | 0.973 |
| 4 days | CD177 + C3AR1 + CD74 + S100A9 + CIITA + TAP2 + CD3D + | 0.805 | 0.637 | 0.972 |
| | CTLA4 | | | |
| 4 days | CD177 + C3AR1 + CD74 + S100A9 + CIITA + TAP2 + CTLA4 | 0.816 | 0.652 | 0.981 |
| 4 days | CD177 + C3AR1 + CD74 + S100A9 + CTLA4 | 0.773 | 0.605 | 0.942 |
| 4 days | CD177 + C3AR1 + CD74 + S100A9 + CX3CR1 | 0.871 | 0.726 | 1 |
| 4 days | CD177 + C3AR1 + CD74 + S100A9 + CX3CR1 + CD3D | 0.887 | 0.75 | 1 |
| 4 days | CD177 + C3AR1 + CD74 + S100A9 + CX3CR1 + CD3D + CTLA4 | 0.887 | 0.759 | 1 |
| 4 days | CD177 + C3AR1 + CD74 + S100A9 + CX3CR1 + CIITA | 0.879 | 0.743 | 1 |
| 4 days | CD177 + C3AR1 + CD74 + S100A9 + CX3CR1 + CIITA + CD3D | 0.887 | 0.755 | 1 |
| 4 days | CD177 + C3AR1 + CD74 + S100A9 + CX3CR1 + CIITA + CD3D + | 0.883 | 0.754 | 1 |
| | CTLA4 | | | |
| 4 days | CD177 + C3AR1 + CD74 + S100A9 + CX3CR1 + CIITA + CTLA4 | 0.875 | 0.738 | 1 |
| 4 days | CD177 + C3AR1 + CD74 + S100A9 + CX3CR1 + CIITA + TAP2 | 0.887 | 0.747 | 1 |
| 4 days | CD177 + C3AR1 + CD74 + S100A9 + CX3CR1 + CIITA + TAP2 + | 0.887 | 0.755 | 1 |
| | CD3D | | | |
| 4 days | CD177 + C3AR1 + CD74 + S100A9 + CX3CR1 + CIITA + TAP2 + | 0.891 | 0.767 | 1 |
| | CD3D + CTLA4 | | | |
| 4 days | CD177 + C3AR1 + CD74 + S100A9 + CX3CR1 + CIITA + TAP2 + | 0.883 | 0.739 | 1 |
| | CTLA4 | | | |
| 4 days | CD177 + C3AR1 + CD74 + S100A9 + CX3CR1 + CTLA4 | 0.867 | 0.721 | 1 |
| 4 days | CD177 + C3AR1 + CD74 + S100A9 + CX3CR1 + TAP2 | 0.883 | 0.743 | 1 |
| 4 days | CD177 + C3AR1 + CD74 + S100A9 + CX3CR1 + TAP2 + CD3D | 0.887 | 0.75 | 1 |
| 4 days | CD177 + C3AR1 + CD74 + S100A9 + CX3CR1 + TAP2 + CD3D + | 0.879 | 0.749 | 1 |
| | CTLA4 | | | |
| 4 days | CD177 + C3AR1 + CD74 + S100A9 + CX3CR1 + TAP2 + CTLA4 | 0.883 | 0.739 | 1 |
| 4 days | CD177 + C3AR1 + CD74 + S100A9 + TAP2 | 0.801 | 0.635 | 0.967 |
| 4 days | CD177 + C3AR1 + CD74 + S100A9 + TAP2 + CD3D | 0.805 | 0.647 | 0.963 |
| 4 days | CD177 + C3AR1 + CD74 + S100A9 + TAP2 + CD3D + CTLA4 | 0.805 | 0.648 | 0.961 |
| 4 days | CD177 + C3AR1 + CD74 + S100A9 + TAP2 + CTLA4 | 0.797 | 0.634 | 0.96 |
| 4 days | CD177 + C3AR1 + CD74 + TAP2 | 0.719 | 0.535 | 0.903 |
| 4 days | CD177 + C3AR1 + CD74 + TAP2 + CD3D | 0.719 | 0.535 | 0.903 |
| 4 days | CD177 + C3AR1 + CD74 + TAP2 + CD3D + CTLA4 | 0.742 | 0.564 | 0.92 |
| 4 days | CD177 + C3AR1 + CD74 + TAP2 + CTLA4 | 0.738 | 0.558 | 0.919 |
| 4 days | CD177 + C3AR1 + CIITA + TAP2 + CD3D + CTLA4 | 0.707 | 0.518 | 0.896 |
| 4 days | CD177 + C3AR1 + CIITA + TAP2 + CTLA4 | 0.699 | 0.509 | 0.889 |
| 4 days | CD177 + C3AR1 + CX3CR1 | 0.703 | 0.515 | 0.891 |
| 4 days | CD177 + C3AR1 + CX3CR1 + CD3D | 0.695 | 0.508 | 0.883 |
| 4 days | CD177 + C3AR1 + CX3CR1 + CD3D + CTLA4 | 0.699 | 0.511 | 0.888 |
| 4 days | CD177 + C3AR1 + CX3CR1 + CIITA | 0.734 | 0.552 | 0.917 |
| 4 days | CD177 + C3AR1 + CX3CR1 + CIITA + CD3D | 0.719 | 0.531 | 0.907 |
| 4 days | CD177 + C3AR1 + CX3CR1 + CIITA + CD3D + CTLA4 | 0.715 | 0.525 | 0.905 |
| 4 days | CD177 + C3AR1 + CX3CR1 + CIITA + CTLA4 | 0.715 | 0.524 | 0.906 |
| 4 days | CD177 + C3AR1 + CX3CR1 + CIITA + TAP2 | 0.773 | 0.611 | 0.936 |
| 4 days | CD177 + C3AR1 + CX3CR1 + CIITA + TAP2 + CD3D | 0.773 | 0.61 | 0.937 |
| 4 days | CD177 + C3AR1 + CX3CR1 + CIITA + TAP2 + CD3D + CTLA4 | 0.77 | 0.605 | 0.934 |
| 4 days | CD177 + C3AR1 + CX3CR1 + CIITA + TAP2 + CTLA4 | 0.762 | 0.594 | 0.929 |
| 4 days | CD177 + C3AR1 + CX3CR1 + CTLA4 | 0.703 | 0.513 | 0.893 |
| 4 days | CD177 + C3AR1 + CX3CR1 + TAP2 | 0.73 | 0.553 | 0.908 |
| 4 days | CD177 + C3AR1 + CX3CR1 + TAP2 + CD3D | 0.746 | 0.575 | 0.917 |
| 4 days | CD177 + C3AR1 + CX3CR1 + TAP2 + CD3D + CTLA4 | 0.758 | 0.59 | 0.926 |
| 4 days | CD177 + C3AR1 + CX3CR1 + TAP2 + CTLA4 | 0.754 | 0.585 | 0.923 |
| 4 days | CD177 + C3AR1 + IFNG | 0.703 | 0.517 | 0.89 |
| 4 days | CD177 + C3AR1 + IFNG + CD3D | 0.707 | 0.523 | 0.891 |
| 4 days | CD177 + C3AR1 + IFNG + CD3D + CTLA4 | 0.711 | 0.526 | 0.896 |
| 4 days | CD177 + C3AR1 + IFNG + CIITA | 0.707 | 0.524 | 0.89 |
| 4 days | CD177 + C3AR1 + IFNG + CIITA + CD3D | 0.699 | 0.514 | 0.884 |
| 4 days | CD177 + C3AR1 + IFNG + CIITA + CD3D + CTLA4 | 0.699 | 0.509 | 0.889 |
| 4 days | CD177 + C3AR1 + IFNG + CIITA + CTLA4 | 0.734 | 0.552 | 0.916 |
| 4 days | CD177 + C3AR1 + IFNG + CIITA + TAP2 | 0.703 | 0.518 | 0.888 |
| 4 days | CD177 + C3AR1 + IFNG + CIITA + TAP2 + CD3D | 0.703 | 0.518 | 0.888 |
| 4 days | CD177 + C3AR1 + IFNG + CIITA + TAP2 + CD3D + CTLA4 | 0.703 | 0.512 | 0.895 |
| 4 days | CD177 + C3AR1 + IFNG + CIITA + TAP2 + CTLA4 | 0.734 | 0.552 | 0.916 |
| 4 days | CD177 + C3AR1 + IFNG + CTLA4 | 0.727 | 0.546 | 0.907 |
| 4 days | CD177 + C3AR1 + IFNG + CX3CR1 | 0.844 | 0.696 | 0.992 |
| 4 days | CD177 + C3AR1 + IFNG + CX3CR1 + CD3D | 0.852 | 0.716 | 0.987 |
| 4 days | CD177 + C3AR1 + IFNG + CX3CR1 + CD3D + CTLA4 | 0.895 | 0.778 | 1 |
| 4 days | CD177 + C3AR1 + IFNG + CX3CR1 + CIITA | 0.863 | 0.722 | 1 |
| 4 days | CD177 + C3AR1 + IFNG + CX3CR1 + CIITA + CD3D | 0.863 | 0.734 | 0.993 |
| 4 days | CD177 + C3AR1 + IFNG + CX3CR1 + CIITA + CD3D + CTLA4 | 0.883 | 0.757 | 1 |
| 4 days | CD177 + C3AR1 + IFNG + CX3CR1 + CIITA + CTLA4 | 0.895 | 0.781 | 1 |
| 4 days | CD177 + C3AR1 + IFNG + CX3CR1 + CIITA + TAP2 | 0.867 | 0.736 | 0.998 |
| 4 days | CD177 + C3AR1 + IFNG + CX3CR1 + CIITA + TAP2 + CD3D | 0.863 | 0.732 | 0.995 |
| 4 days | CD177 + C3AR1 + IFNG + CX3CR1 + CIITA + TAP2 + CD3D + | 0.879 | 0.75 | 1 |
| | CTLA4 | | | |
| 4 days | CD177 + C3AR1 + IFNG + CX3CR1 + CIITA + TAP2 + CTLA4 | 0.875 | 0.746 | 1 |
| 4 days | CD177 + C3AR1 + IFNG + CX3CR1 + CTLA4 | 0.883 | 0.765 | 1 |
| 4 days | CD177 + C3AR1 + IFNG + CX3CR1 + TAP2 | 0.855 | 0.722 | 0.989 |
| 4 days | CD177 + C3AR1 + IFNG + CX3CR1 + TAP2 + CD3D | 0.859 | 0.731 | 0.987 |
| 4 days | CD177 + C3AR1 + IFNG + CX3CR1 + TAP2 + CD3D + CTLA4 | 0.883 | 0.757 | 1 |
| 4 days | CD177 + C3AR1 + IFNG + CX3CR1 + TAP2 + CTLA4 | 0.879 | 0.752 | 1 |
| 4 days | CD177 + C3AR1 + IFNG + S100A9 | 0.789 | 0.624 | 0.954 |
| 4 days | CD177 + C3AR1 + IFNG + S100A9 + CD3D | 0.781 | 0.615 | 0.947 |
| 4 days | CD177 + C3AR1 + IFNG + S100A9 + CD3D + CTLA4 | 0.785 | 0.62 | 0.95 |
| 4 days | CD177 + C3AR1 + IFNG + S100A9 + CIITA | 0.793 | 0.625 | 0.961 |
| 4 days | CD177 + C3AR1 + IFNG + S100A9 + CIITA + CD3D | 0.789 | 0.624 | 0.954 |
| 4 days | CD177 + C3AR1 + IFNG + S100A9 + CIITA + CD3D + CTLA4 | 0.785 | 0.62 | 0.951 |
| 4 days | CD177 + C3AR1 + IFNG + S100A9 + CIITA + CTLA4 | 0.785 | 0.619 | 0.952 |
| 4 days | CD177 + C3AR1 + IFNG + S100A9 + CIITA + TAP2 | 0.801 | 0.628 | 0.974 |
| 4 days | CD177 + C3AR1 + IFNG + S100A9 + CIITA + TAP2 + CD3D | 0.797 | 0.636 | 0.957 |
| 4 days | CD177 + C3AR1 + IFNG + S100A9 + CIITA + TAP2 + CD3D + | 0.797 | 0.635 | 0.959 |
| | CTLA4 | | | |
| 4 days | CD177 + C3AR1 + IFNG + S100A9 + CIITA + TAP2 + CTLA4 | 0.812 | 0.653 | 0.972 |
| 4 days | CD177 + C3AR1 + IFNG + S100A9 + CTLA4 | 0.77 | 0.6 | 0.939 |
| 4 days | CD177 + C3AR1 + IFNG + S100A9 + CX3CR1 | 0.93 | 0.838 | 1 |
| 4 days | CD177 + C3AR1 + IFNG + S100A9 + CX3CR1 + CD3D | 0.941 | 0.866 | 1 |
| 4 days | CD177 + C3AR1 + IFNG + S100A9 + CX3CR1 + CD3D + CTLA4 | 0.957 | 0.897 | 1 |
| 4 days | CD177 + C3AR1 + IFNG + S100A9 + CX3CR1 + CIITA | 0.93 | 0.838 | 1 |
| 4 days | CD177 + C3AR1 + IFNG + S100A9 + CX3CR1 + CIITA + CD3D | 0.941 | 0.866 | 1 |
| 4 days | CD177 + C3AR1 + IFNG + S100A9 + CX3CR1 + CIITA + CD3D + | 0.957 | 0.897 | 1 |
| | CTLA4 | | | |
| 4 days | CD177 + C3AR1 + IFNG + S100A9 + CX3CR1 + CIITA + CTLA4 | 0.945 | 0.871 | 1 |
| 4 days | CD177 + C3AR1 + IFNG + S100A9 + CX3CR1 + CIITA + TAP2 | 0.934 | 0.845 | 1 |
| 4 days | CD177 + C3AR1 + IFNG + S100A9 + CX3CR1 + CIITA + TAP2 + | 0.949 | 0.882 | 1 |
| | CD3D | | | |
| 4 days | CD177 + C3AR1 + IFNG + S100A9 + CX3CR1 + CIITA + TAP2 + | 0.953 | 0.889 | 1 |
| | CD3D + CTLA4 | | | |
| 4 days | CD177 + C3AR1 + IFNG + S100A9 + CX3CR1 + CIITA + TAP2 + | 0.941 | 0.863 | 1 |
| | CTLA4 | | | |
| 4 days | CD177 + C3AR1 + IFNG + S100A9 + CX3CR1 + CTLA4 | 0.941 | 0.864 | 1 |
| 4 days | CD177 + C3AR1 + IFNG + S100A9 + CX3CR1 + TAP2 | 0.934 | 0.845 | 1 |
| 4 days | CD177 + C3AR1 + IFNG + S100A9 + CX3CR1 + TAP2 + CD3D | 0.949 | 0.882 | 1 |
| 4 days | CD177 + C3AR1 + IFNG + S100A9 + CX3CR1 + TAP2 + CD3D + | 0.953 | 0.889 | 1 |
| | CTLA4 | | | |
| 4 days | CD177 + C3AR1 + IFNG + S100A9 + CX3CR1 + TAP2 + CTLA4 | 0.938 | 0.852 | 1 |
| 4 days | CD177 + C3AR1 + IFNG + S100A9 + TAP2 | 0.801 | 0.635 | 0.967 |
| 4 days | CD177 + C3AR1 + IFNG + S100A9 + TAP2 + CD3D | 0.801 | 0.642 | 0.959 |
| 4 days | CD177 + C3AR1 + IFNG + S100A9 + TAP2 + CD3D + CTLA4 | 0.805 | 0.648 | 0.961 |
| 4 days | CD177 + C3AR1 + IFNG + S100A9 + TAP2 + CTLA4 | 0.785 | 0.621 | 0.949 |
| 4 days | CD177 + C3AR1 + IFNG + TAP2 | 0.719 | 0.535 | 0.902 |
| 4 days | CD177 + C3AR1 + IFNG + TAP2 + CD3D | 0.719 | 0.536 | 0.902 |
| 4 days | CD177 + C3AR1 + IFNG + TAP2 + CD3D + CTLA4 | 0.707 | 0.521 | 0.893 |
| 4 days | CD177 + C3AR1 + IFNG + TAP2 + CTLA4 | 0.727 | 0.546 | 0.907 |
| 4 days | CD177 + C3AR1 + S100A9 | 0.781 | 0.614 | 0.948 |
| 4 days | CD177 + C3AR1 + S100A9 + CD3D | 0.781 | 0.617 | 0.946 |
| 4 days | CD177 + C3AR1 + S100A9 + CD3D + CTLA4 | 0.781 | 0.615 | 0.947 |
| 4 days | CD177 + C3AR1 + S100A9 + CIITA | 0.793 | 0.625 | 0.961 |
| 4 days | CD177 + C3AR1 + S100A9 + CIITA + CD3D | 0.781 | 0.615 | 0.948 |
| 4 days | CD177 + C3AR1 + S100A9 + CIITA + CD3D + CTLA4 | 0.801 | 0.64 | 0.961 |
| 4 days | CD177 + C3AR1 + S100A9 + CIITA + CTLA4 | 0.797 | 0.632 | 0.962 |
| 4 days | CD177 + C3AR1 + S100A9 + CIITA + TAP2 | 0.805 | 0.632 | 0.977 |
| 4 days | CD177 + C3AR1 + S100A9 + CIITA + TAP2 + CD3D | 0.809 | 0.647 | 0.971 |
| 4 days | CD177 + C3AR1 + S100A9 + CIITA + TAP2 + CD3D + CTLA4 | 0.805 | 0.642 | 0.967 |
| 4 days | CD177 + C3AR1 + S100A9 + CIITA + TAP2 + CTLA4 | 0.816 | 0.656 | 0.976 |
| 4 days | CD177 + C3AR1 + S100A9 + CTLA4 | 0.77 | 0.6 | 0.939 |
| 4 days | CD177 + C3AR1 + S100A9 + CX3CR1 | 0.859 | 0.711 | 1 |
| 4 days | CD177 + C3AR1 + S100A9 + CX3CR1 + CD3D | 0.887 | 0.755 | 1 |
| 4 days | CD177 + C3AR1 + S100A9 + CX3CR1 + CD3D + CTLA4 | 0.879 | 0.749 | 1 |
| 4 days | CD177 + C3AR1 + S100A9 + CX3CR1 + CIITA | 0.879 | 0.743 | 1 |
| 4 days | CD177 + C3AR1 + S100A9 + CX3CR1 + CIITA + CD3D | 0.887 | 0.755 | 1 |
| 4 days | CD177 + C3AR1 + S100A9 + CX3CR1 + CIITA + CD3D + CTLA4 | 0.887 | 0.762 | 1 |
| 4 days | CD177 + C3AR1 + S100A9 + CX3CR1 + CIITA + CTLA4 | 0.871 | 0.729 | 1 |
| 4 days | CD177 + C3AR1 + S100A9 + CX3CR1 + CIITA + TAP2 | 0.883 | 0.743 | 1 |
| 4 days | CD177 + C3AR1 + S100A9 + CX3CR1 + CIITA + TAP2 + CD3D | 0.887 | 0.755 | 1 |
| 4 days | CD177 + C3AR1 + S100A9 + CX3CR1 + CIITA + TAP2 + CD3D + | 0.887 | 0.76 | 1 |
| | CTLA4 | | | |
| 4 days | CD177 + C3AR1 + S100A9 + CX3CR1 + CIITA + TAP2 + CTLA4 | 0.879 | 0.735 | 1 |
| 4 days | CD177 + C3AR1 + S100A9 + CX3CR1 + CTLA4 | 0.859 | 0.712 | 1 |
| 4 days | CD177 + C3AR1 + S100A9 + CX3CR1 + TAP2 | 0.883 | 0.743 | 1 |
| 4 days | CD177 + C3AR1 + S100A9 + CX3CR1 + TAP2 + CD3D | 0.887 | 0.755 | 1 |
| 4 days | CD177 + C3AR1 + S100A9 + CX3CR1 + TAP2 + CD3D + CTLA4 | 0.879 | 0.751 | 1 |
| 4 days | CD177 + C3AR1 + S100A9 + CX3CR1 + TAP2 + CTLA4 | 0.883 | 0.739 | 1 |
| 4 days | CD177 + C3AR1 + S100A9 + TAP2 | 0.801 | 0.635 | 0.967 |
| 4 days | CD177 + C3AR1 + S100A9 + TAP2 + CD3D | 0.801 | 0.642 | 0.959 |
| 4 days | CD177 + C3AR1 + S100A9 + TAP2 + CD3D + CTLA4 | 0.805 | 0.648 | 0.961 |
| 4 days | CD177 + C3AR1 + S100A9 + TAP2 + CTLA4 | 0.797 | 0.634 | 0.96 |
| 4 days | CD177 + C3AR1 + TAP2 | 0.699 | 0.511 | 0.888 |
| 4 days | CD177 + C3AR1 + TAP2 + CD3D | 0.695 | 0.504 | 0.886 |
| 4 days | CD177 + C3AR1 + TAP2 + CD3D + CTLA4 | 0.703 | 0.512 | 0.894 |
| 4 days | CD177 + C3AR1 + TAP2 + CTLA4 | 0.699 | 0.509 | 0.89 |
| 4 days | CD177 + CD3D + CTLA4 | 0.695 | 0.504 | 0.887 |
| 4 days | CD177 + CD74 + CD3D | 0.727 | 0.541 | 0.912 |
| 4 days | CD177 + CD74 + CD3D + CTLA4 | 0.719 | 0.53 | 0.908 |
| 4 days | CD177 + CD74 + CIITA | 0.723 | 0.537 | 0.908 |
| 4 days | CD177 + CD74 + CIITA + CD3D | 0.723 | 0.537 | 0.908 |
| 4 days | CD177 + CD74 + CIITA + CD3D + CTLA4 | 0.719 | 0.53 | 0.908 |
| 4 days | CD177 + CD74 + CIITA + CTLA4 | 0.73 | 0.543 | 0.918 |
| 4 days | CD177 + CD74 + CIITA + TAP2 | 0.727 | 0.541 | 0.912 |
| 4 days | CD177 + CD74 + CIITA + TAP2 + CD3D | 0.727 | 0.541 | 0.912 |
| 4 days | CD177 + CD74 + CIITA + TAP2 + CD3D + CTLA4 | 0.719 | 0.528 | 0.91 |
| 4 days | CD177 + CD74 + CIITA + TAP2 + CTLA4 | 0.727 | 0.539 | 0.915 |
| 4 days | CD177 + CD74 + CTLA4 | 0.73 | 0.543 | 0.918 |
| 4 days | CD177 + CD74 + CX3CR1 | 0.758 | 0.581 | 0.934 |
| 4 days | CD177 + CD74 + CX3CR1 + CD3D | 0.75 | 0.573 | 0.927 |
| 4 days | CD177 + CD74 + CX3CR1 + CD3D + CTLA4 | 0.75 | 0.572 | 0.928 |
| 4 days | CD177 + CD74 + CX3CR1 + CIITA | 0.75 | 0.566 | 0.934 |
| 4 days | CD177 + CD74 + CX3CR1 + CIITA + CD3D | 0.742 | 0.554 | 0.93 |
| 4 days | CD177 + CD74 + CX3CR1 + CIITA + CD3D + CTLA4 | 0.75 | 0.566 | 0.934 |
| 4 days | CD177 + CD74 + CX3CR1 + CIITA + CTLA4 | 0.734 | 0.545 | 0.924 |
| 4 days | CD177 + CD74 + CX3CR1 + CIITA + TAP2 | 0.766 | 0.588 | 0.943 |
| 4 days | CD177 + CD74 + CX3CR1 + CIITA + TAP2 + CD3D | 0.75 | 0.568 | 0.932 |
| 4 days | CD177 + CD74 + CX3CR1 + CIITA + TAP2 + CD3D + CTLA4 | 0.758 | 0.583 | 0.933 |
| 4 days | CD177 + CD74 + CX3CR1 + CIITA + TAP2 + CTLA4 | 0.766 | 0.593 | 0.939 |
| 4 days | CD177 + CD74 + CX3CR1 + CTLA4 | 0.742 | 0.558 | 0.927 |
| 4 days | CD177 + CD74 + CX3CR1 + TAP2 | 0.758 | 0.582 | 0.934 |
| 4 days | CD177 + CD74 + CX3CR1 + TAP2 + CD3D | 0.754 | 0.578 | 0.93 |
| 4 days | CD177 + CD74 + CX3CR1 + TAP2 + CD3D + CTLA4 | 0.777 | 0.613 | 0.942 |
| 4 days | CD177 + CD74 + CX3CR1 + TAP2 + CTLA4 | 0.789 | 0.628 | 0.95 |
| 4 days | CD177 + CD74 + IFNG | 0.707 | 0.521 | 0.893 |
| 4 days | CD177 + CD74 + IFNG + CD3D | 0.711 | 0.526 | 0.896 |
| 4 days | CD177 + CD74 + IFNG + CD3D + CTLA4 | 0.699 | 0.511 | 0.887 |
| 4 days | CD177 + CD74 + IFNG + CIITA | 0.703 | 0.517 | 0.89 |
| 4 days | CD177 + CD74 + IFNG + CIITA + CD3D | 0.703 | 0.517 | 0.889 |
| 4 days | CD177 + CD74 + IFNG + CIITA + CTLA4 | 0.699 | 0.508 | 0.89 |
| 4 days | CD177 + CD74 + IFNG + CIITA + TAP2 | 0.703 | 0.517 | 0.89 |
| 4 days | CD177 + CD74 + IFNG + CIITA + TAP2 + CD3D | 0.703 | 0.517 | 0.89 |
| 4 days | CD177 + CD74 + IFNG + CIITA + TAP2 + CTLA4 | 0.699 | 0.511 | 0.887 |
| 4 days | CD177 + CD74 + IFNG + CTLA4 | 0.703 | 0.516 | 0.89 |
| 4 days | CD177 + CD74 + IFNG + CX3CR1 | 0.855 | 0.714 | 0.997 |
| 4 days | CD177 + CD74 + IFNG + CX3CR1 + CD3D | 0.863 | 0.736 | 0.991 |
| 4 days | CD177 + CD74 + IFNG + CX3CR1 + CD3D + CTLA4 | 0.891 | 0.778 | 1 |
| 4 days | CD177 + CD74 + IFNG + CX3CR1 + CIITA | 0.875 | 0.748 | 1 |
| 4 days | CD177 + CD74 + IFNG + CX3CR1 + CIITA + CD3D | 0.879 | 0.764 | 0.994 |
| 4 days | CD177 + CD74 + IFNG + CX3CR1 + CIITA + CD3D + CTLA4 | 0.891 | 0.778 | 1 |
| 4 days | CD177 + CD74 + IFNG + CX3CR1 + CIITA + CTLA4 | 0.895 | 0.784 | 1 |
| 4 days | CD177 + CD74 + IFNG + CX3CR1 + CIITA + TAP2 | 0.871 | 0.744 | 0.998 |
| 4 days | CD177 + CD74 + IFNG + CX3CR1 + CIITA + TAP2 + CD3D | 0.867 | 0.746 | 0.988 |
| 4 days | CD177 + CD74 + IFNG + CX3CR1 + CIITA + TAP2 + CD3D + | 0.883 | 0.768 | 0.997 |
| | CTLA4 | | | |
| 4 days | CD177 + CD74 + IFNG + CX3CR1 + CIITA + TAP2 + CTLA4 | 0.883 | 0.768 | 0.997 |
| 4 days | CD177 + CD74 + IFNG + CX3CR1 + CTLA4 | 0.895 | 0.784 | 1 |
| 4 days | CD177 + CD74 + IFNG + CX3CR1 + TAP2 | 0.855 | 0.719 | 0.992 |
| 4 days | CD177 + CD74 + IFNG + CX3CR1 + TAP2 + CD3D | 0.875 | 0.753 | 0.997 |
| 4 days | CD177 + CD74 + IFNG + CX3CR1 + TAP2 + CD3D + CTLA4 | 0.883 | 0.768 | 0.997 |
| 4 days | CD177 + CD74 + IFNG + CX3CR1 + TAP2 + CTLA4 | 0.883 | 0.768 | 0.997 |
| 4 days | CD177 + CD74 + IFNG + S100A9 | 0.754 | 0.573 | 0.935 |
| 4 days | CD177 + CD74 + IFNG + S100A9 + CD3D | 0.762 | 0.583 | 0.94 |
| 4 days | CD177 + CD74 + IFNG + S100A9 + CD3D + CTLA4 | 0.758 | 0.58 | 0.936 |
| 4 days | CD177 + CD74 + IFNG + S100A9 + CIITA | 0.734 | 0.544 | 0.925 |
| 4 days | CD177 + CD74 + IFNG + S100A9 + CIITA + CD3D | 0.734 | 0.544 | 0.925 |
| 4 days | CD177 + CD74 + IFNG + S100A9 + CIITA + CD3D + CTLA4 | 0.742 | 0.562 | 0.923 |
| 4 days | CD177 + CD74 + IFNG + S100A9 + CIITA + CTLA4 | 0.738 | 0.556 | 0.92 |
| 4 days | CD177 + CD74 + IFNG + S100A9 + CIITA + TAP2 | 0.73 | 0.534 | 0.927 |
| 4 days | CD177 + CD74 + IFNG + S100A9 + CIITA + TAP2 + CD3D | 0.73 | 0.532 | 0.928 |
| 4 days | CD177 + CD74 + IFNG + S100A9 + CIITA + TAP2 + CD3D + | 0.723 | 0.529 | 0.916 |
| | CTLA4 | | | |
| 4 days | CD177 + CD74 + IFNG + S100A9 + CIITA + TAP2 + CTLA4 | 0.727 | 0.531 | 0.922 |
| 4 days | CD177 + CD74 + IFNG + S100A9 + CTLA4 | 0.754 | 0.573 | 0.935 |
| 4 days | CD177 + CD74 + IFNG + S100A9 + CX3CR1 | 0.93 | 0.838 | 1 |
| 4 days | CD177 + CD74 + IFNG + S100A9 + CX3CR1 + CD3D | 0.945 | 0.875 | 1 |
| 4 days | CD177 + CD74 + IFNG + S100A9 + CX3CR1 + CD3D + CTLA4 | 0.949 | 0.88 | 1 |
| 4 days | CD177 + CD74 + IFNG + S100A9 + CX3CR1 + CIITA | 0.93 | 0.838 | 1 |
| 4 days | CD177 + CD74 + IFNG + S100A9 + CX3CR1 + CIITA + CD3D | 0.945 | 0.872 | 1 |
| 4 days | CD177 + CD74 + IFNG + S100A9 + CX3CR1 + CIITA + CD3D + | 0.953 | 0.889 | 1 |
| | CTLA4 | | | |
| 4 days | CD177 + CD74 + IFNG + S100A9 + CX3CR1 + CIITA + CTLA4 | 0.961 | 0.905 | 1 |
| 4 days | CD177 + CD74 + IFNG + S100A9 + CX3CR1 + CIITA + TAP2 | 0.934 | 0.845 | 1 |
| 4 days | CD177 + CD74 + IFNG + S100A9 + CX3CR1 + CIITA + TAP2 + | 0.949 | 0.882 | 1 |
| | CD3D | | | |
| 4 days | CD177 + CD74 + IFNG + S100A9 + CX3CR1 + CIITA + TAP2 + | 0.949 | 0.882 | 1 |
| | CD3D + CTLA4 | | | |
| 4 days | CD177 + CD74 + IFNG + S100A9 + CX3CR1 + CIITA + TAP2 + | 0.953 | 0.889 | 1 |
| | CTLA4 | | | |
| 4 days | CD177 + CD74 + IFNG + S100A9 + CX3CR1 + CTLA4 | 0.945 | 0.871 | 1 |
| 4 days | CD177 + CD74 + IFNG + S100A9 + CX3CR1 + TAP2 | 0.934 | 0.845 | 1 |
| 4 days | CD177 + CD74 + IFNG + S100A9 + CX3CR1 + TAP2 + CD3D | 0.949 | 0.882 | 1 |
| 4 days | CD177 + CD74 + IFNG + S100A9 + CX3CR1 + TAP2 + CD3D + | 0.949 | 0.882 | 1 |
| | CTLA4 | | | |
| 4 days | CD177 + CD74 + IFNG + S100A9 + CX3CR1 + TAP2 + CTLA4 | 0.938 | 0.849 | 1 |
| 4 days | CD177 + CD74 + IFNG + S100A9 + TAP2 | 0.758 | 0.574 | 0.942 |
| 4 days | CD177 + CD74 + IFNG + S100A9 + TAP2 + CD3D | 0.754 | 0.569 | 0.939 |
| 4 days | CD177 + CD74 + IFNG + S100A9 + TAP2 + CD3D + CTLA4 | 0.758 | 0.575 | 0.94 |
| 4 days | CD177 + CD74 + IFNG + S100A9 + TAP2 + CTLA4 | 0.746 | 0.557 | 0.935 |
| 4 days | CD177 + CD74 + IFNG + TAP2 | 0.707 | 0.522 | 0.892 |
| 4 days | CD177 + CD74 + IFNG + TAP2 + CD3D | 0.707 | 0.522 | 0.892 |
| 4 days | CD177 + CD74 + IFNG + TAP2 + CD3D + CTLA4 | 0.699 | 0.511 | 0.888 |
| 4 days | CD177 + CD74 + IFNG + TAP2 + CTLA4 | 0.719 | 0.536 | 0.901 |
| 4 days | CD177 + CD74 + S100A9 | 0.754 | 0.573 | 0.935 |
| 4 days | CD177 + CD74 + S100A9 + CD3D | 0.758 | 0.579 | 0.937 |
| 4 days | CD177 + CD74 + S100A9 + CD3D + CTLA4 | 0.77 | 0.592 | 0.947 |
| 4 days | CD177 + CD74 + S100A9 + CIITA | 0.742 | 0.554 | 0.931 |
| 4 days | CD177 + CD74 + S100A9 + CIITA + CD3D | 0.738 | 0.55 | 0.927 |
| 4 days | CD177 + CD74 + S100A9 + CIITA + CD3D + CTLA4 | 0.754 | 0.573 | 0.935 |
| 4 days | CD177 + CD74 + S100A9 + CIITA + CTLA4 | 0.746 | 0.564 | 0.928 |
| 4 days | CD177 + CD74 + S100A9 + CIITA + TAP2 | 0.738 | 0.544 | 0.932 |
| 4 days | CD177 + CD74 + S100A9 + CIITA + TAP2 + CD3D | 0.73 | 0.534 | 0.927 |
| 4 days | CD177 + CD74 + S100A9 + CIITA + TAP2 + CD3D + CTLA4 | 0.746 | 0.559 | 0.933 |
| 4 days | CD177 + CD74 + S100A9 + CIITA + TAP2 + CTLA4 | 0.746 | 0.559 | 0.933 |
| 4 days | CD177 + CD74 + S100A9 + CTLA4 | 0.75 | 0.567 | 0.933 |
| 4 days | CD177 + CD74 + S100A9 + CX3CR1 | 0.852 | 0.707 | 0.996 |
| 4 days | CD177 + CD74 + S100A9 + CX3CR1 + CD3D | 0.859 | 0.721 | 0.998 |
| 4 days | CD177 + CD74 + S100A9 + CX3CR1 + CD3D + CTLA4 | 0.863 | 0.729 | 0.997 |
| 4 days | CD177 + CD74 + S100A9 + CX3CR1 + CIITA | 0.848 | 0.704 | 0.992 |
| 4 days | CD177 + CD74 + S100A9 + CX3CR1 + CIITA + CD3D | 0.859 | 0.721 | 0.998 |
| 4 days | CD177 + CD74 + S100A9 + CX3CR1 + CIITA + CD3D + CTLA4 | 0.844 | 0.702 | 0.986 |
| 4 days | CD177 + CD74 + S100A9 + CX3CR1 + CIITA + CTLA4 | 0.855 | 0.712 | 0.999 |
| 4 days | CD177 + CD74 + S100A9 + CX3CR1 + CIITA + TAP2 | 0.867 | 0.732 | 1 |
| 4 days | CD177 + CD74 + S100A9 + CX3CR1 + CIITA + TAP2 + CD3D | 0.859 | 0.721 | 0.998 |
| 4 days | CD177 + CD74 + S100A9 + CX3CR1 + CIITA + TAP2 + CD3D + | 0.859 | 0.726 | 0.993 |
| | CTLA4 | | | |
| 4 days | CD177 + CD74 + S100A9 + CX3CR1 + CIITA + TAP2 + CTLA4 | 0.859 | 0.718 | 1 |
| 4 days | CD177 + CD74 + S100A9 + CX3CR1 + CTLA4 | 0.855 | 0.713 | 0.998 |
| 4 days | CD177 + CD74 + S100A9 + CX3CR1 + TAP2 | 0.859 | 0.719 | 1 |
| 4 days | CD177 + CD74 + S100A9 + CX3CR1 + TAP2 + CD3D | 0.859 | 0.721 | 0.998 |
| 4 days | CD177 + CD74 + S100A9 + CX3CR1 + TAP2 + CD3D + CTLA4 | 0.859 | 0.724 | 0.995 |
| 4 days | CD177 + CD74 + S100A9 + CX3CR1 + TAP2 + CTLA4 | 0.859 | 0.716 | 1 |
| 4 days | CD177 + CD74 + S100A9 + TAP2 | 0.75 | 0.562 | 0.938 |
| 4 days | CD177 + CD74 + S100A9 + TAP2 + CD3D | 0.754 | 0.569 | 0.939 |
| 4 days | CD177 + CD74 + S100A9 + TAP2 + CD3D + CTLA4 | 0.754 | 0.57 | 0.938 |
| 4 days | CD177 + CD74 + S100A9 + TAP2 + CTLA4 | 0.75 | 0.565 | 0.935 |
| 4 days | CD177 + CD74 + TAP2 | 0.734 | 0.55 | 0.919 |
| 4 days | CD177 + CD74 + TAP2 + CD3D | 0.727 | 0.54 | 0.913 |
| 4 days | CD177 + CD74 + TAP2 + CD3D + CTLA4 | 0.727 | 0.538 | 0.916 |
| 4 days | CD177 + CD74 + TAP2 + CTLA4 | 0.727 | 0.539 | 0.915 |
| 4 days | CD177 + CIITA + CD3D | 0.703 | 0.515 | 0.891 |
| 4 days | CD177 + CIITA + CD3D + CTLA4 | 0.719 | 0.532 | 0.905 |
| 4 days | CD177 + CIITA + CTLA4 | 0.711 | 0.524 | 0.898 |
| 4 days | CD177 + CIITA + TAP2 | 0.707 | 0.517 | 0.897 |
| 4 days | CD177 + CIITA + TAP2 + CD3D | 0.711 | 0.522 | 0.9 |
| 4 days | CD177 + CIITA + TAP2 + CD3D + CTLA4 | 0.719 | 0.531 | 0.907 |
| 4 days | CD177 + CIITA + TAP2 + CTLA4 | 0.688 | 0.493 | 0.882 |
| 4 days | CD177 + CX3CR1 + CD3D | 0.695 | 0.507 | 0.884 |
| 4 days | CD177 + CX3CR1 + CD3D + CTLA4 | 0.715 | 0.53 | 0.899 |
| 4 days | CD177 + CX3CR1 + CIITA | 0.742 | 0.56 | 0.924 |
| 4 days | CD177 + CX3CR1 + CIITA + CD3D | 0.738 | 0.553 | 0.923 |
| 4 days | CD177 + CX3CR1 + CIITA + CD3D + CTLA4 | 0.734 | 0.549 | 0.92 |
| 4 days | CD177 + CX3CR1 + CIITA + CTLA4 | 0.734 | 0.547 | 0.921 |
| 4 days | CD177 + CX3CR1 + CIITA + TAP2 | 0.762 | 0.592 | 0.931 |
| 4 days | CD177 + CX3CR1 + CIITA + TAP2 + CD3D | 0.746 | 0.571 | 0.921 |
| 4 days | CD177 + CX3CR1 + CIITA + TAP2 + CD3D + CTLA4 | 0.758 | 0.588 | 0.928 |
| 4 days | CD177 + CX3CR1 + CIITA + TAP2 + CTLA4 | 0.754 | 0.583 | 0.925 |
| 4 days | CD177 + CX3CR1 + CTLA4 | 0.707 | 0.517 | 0.897 |
| 4 days | CD177 + CX3CR1 + TAP2 | 0.734 | 0.558 | 0.911 |
| 4 days | CD177 + CX3CR1 + TAP2 + CD3D | 0.73 | 0.555 | 0.906 |
| 4 days | CD177 + CX3CR1 + TAP2 + CD3D + CTLA4 | 0.746 | 0.575 | 0.917 |
| 4 days | CD177 + CX3CR1 + TAP2 + CTLA4 | 0.742 | 0.57 | 0.915 |
| 4 days | CD177 + IFNG + CD3D | 0.691 | 0.5 | 0.883 |
| 4 days | CD177 + IFNG + CD3D + CTLA4 | 0.699 | 0.513 | 0.886 |
| 4 days | CD177 + IFNG + CIITA | 0.715 | 0.532 | 0.898 |
| 4 days | CD177 + IFNG + CIITA + CD3D | 0.719 | 0.536 | 0.901 |
| 4 days | CD177 + IFNG + CIITA + CD3D + CTLA4 | 0.691 | 0.502 | 0.881 |
| 4 days | CD177 + IFNG + CIITA + CTLA4 | 0.699 | 0.508 | 0.89 |
| 4 days | CD177 + IFNG + CIITA + TAP2 | 0.715 | 0.532 | 0.898 |
| 4 days | CD177 + IFNG + CIITA + TAP2 + CD3D | 0.719 | 0.536 | 0.901 |
| 4 days | CD177 + IFNG + CIITA + TAP2 + CD3D + CTLA4 | 0.695 | 0.506 | 0.885 |
| 4 days | CD177 + IFNG + CIITA + TAP2 + CTLA4 | 0.707 | 0.518 | 0.896 |
| 4 days | CD177 + IFNG + CTLA4 | 0.688 | 0.495 | 0.88 |
| 4 days | CD177 + IFNG + CX3CR1 | 0.848 | 0.704 | 0.991 |
| 4 days | CD177 + IFNG + CX3CR1 + CD3D | 0.855 | 0.722 | 0.989 |
| 4 days | CD177 + IFNG + CX3CR1 + CD3D + CTLA4 | 0.867 | 0.74 | 0.994 |
| 4 days | CD177 + IFNG + CX3CR1 + CIITA | 0.859 | 0.719 | 1 |
| 4 days | CD177 + IFNG + CX3CR1 + CIITA + CD3D | 0.859 | 0.728 | 0.99 |
| 4 days | CD177 + IFNG + CX3CR1 + CIITA + CD3D + CTLA4 | 0.875 | 0.749 | 1 |
| 4 days | CD177 + IFNG + CX3CR1 + CIITA + CTLA4 | 0.875 | 0.749 | 1 |
| 4 days | CD177 + IFNG + CX3CR1 + CIITA + TAP2 | 0.867 | 0.736 | 0.998 |
| 4 days | CD177 + IFNG + CX3CR1 + CIITA + TAP2 + CD3D | 0.859 | 0.726 | 0.993 |
| 4 days | CD177 + IFNG + CX3CR1 + CIITA + TAP2 + CD3D + CTLA4 | 0.875 | 0.748 | 1 |
| 4 days | CD177 + IFNG + CX3CR1 + CIITA + TAP2 + CTLA4 | 0.871 | 0.739 | 1 |
| 4 days | CD177 + IFNG + CX3CR1 + CTLA4 | 0.871 | 0.748 | 0.995 |
| 4 days | CD177 + IFNG + CX3CR1 + TAP2 | 0.832 | 0.692 | 0.972 |
| 4 days | CD177 + IFNG + CX3CR1 + TAP2 + CD3D | 0.848 | 0.714 | 0.981 |
| 4 days | CD177 + IFNG + CX3CR1 + TAP2 + CD3D + CTLA4 | 0.883 | 0.761 | 1 |
| 4 days | CD177 + IFNG + CX3CR1 + TAP2 + CTLA4 | 0.875 | 0.748 | 1 |
| 4 days | CD177 + IFNG + S100A9 | 0.75 | 0.567 | 0.933 |
| 4 days | CD177 + IFNG + S100A9 + CD3D | 0.75 | 0.567 | 0.933 |
| 4 days | CD177 + IFNG + S100A9 + CD3D + CTLA4 | 0.762 | 0.586 | 0.938 |
| 4 days | CD177 + IFNG + S100A9 + CIITA | 0.734 | 0.545 | 0.924 |
| 4 days | CD177 + IFNG + S100A9 + CIITA + CD3D | 0.738 | 0.55 | 0.927 |
| 4 days | CD177 + IFNG + S100A9 + CIITA + CD3D + CTLA4 | 0.75 | 0.572 | 0.928 |
| 4 days | CD177 + IFNG + S100A9 + CIITA + CTLA4 | 0.742 | 0.561 | 0.923 |
| 4 days | CD177 + IFNG + S100A9 + CIITA + TAP2 | 0.73 | 0.534 | 0.927 |
| 4 days | CD177 + IFNG + S100A9 + CIITA + TAP2 + CD3D | 0.723 | 0.522 | 0.924 |
| 4 days | CD177 + IFNG + S100A9 + CIITA + TAP2 + CD3D + CTLA4 | 0.727 | 0.536 | 0.918 |
| 4 days | CD177 + IFNG + S100A9 + CIITA + TAP2 + CTLA4 | 0.727 | 0.531 | 0.922 |
| 4 days | CD177 + IFNG + S100A9 + CTLA4 | 0.746 | 0.561 | 0.931 |
| 4 days | CD177 + IFNG + S100A9 + CX3CR1 | 0.926 | 0.831 | 1 |
| 4 days | CD177 + IFNG + S100A9 + CX3CR1 + CD3D | 0.949 | 0.88 | 1 |
| 4 days | CD177 + IFNG + S100A9 + CX3CR1 + CD3D + CTLA4 | 0.941 | 0.861 | 1 |
| 4 days | CD177 + IFNG + S100A9 + CX3CR1 + CIITA | 0.926 | 0.831 | 1 |
| 4 days | CD177 + IFNG + S100A9 + CX3CR1 + CIITA + CD3D | 0.945 | 0.873 | 1 |
| 4 days | CD177 + IFNG + S100A9 + CX3CR1 + CIITA + CD3D + CTLA4 | 0.945 | 0.871 | 1 |
| 4 days | CD177 + IFNG + S100A9 + CX3CR1 + CIITA + CTLA4 | 0.945 | 0.871 | 1 |
| 4 days | CD177 + IFNG + S100A9 + CX3CR1 + CIITA + TAP2 | 0.93 | 0.838 | 1 |
| 4 days | CD177 + IFNG + S100A9 + CX3CR1 + CIITA + TAP2 + CD3D | 0.953 | 0.883 | 1 |
| 4 days | CD177 + IFNG + S100A9 + CX3CR1 + CIITA + TAP2 + CD3D + | 0.949 | 0.877 | 1 |
| | CTLA4 | | | |
| 4 days | CD177 + IFNG + S100A9 + CX3CR1 + CIITA + TAP2 + CTLA4 | 0.941 | 0.861 | 1 |
| 4 days | CD177 + IFNG + S100A9 + CX3CR1 + CTLA4 | 0.941 | 0.864 | 1 |
| 4 days | CD177 + IFNG + S100A9 + CX3CR1 + TAP2 | 0.93 | 0.838 | 1 |
| 4 days | CD177 + IFNG + S100A9 + CX3CR1 + TAP2 + CD3D | 0.953 | 0.883 | 1 |
| 4 days | CD177 + IFNG + S100A9 + CX3CR1 + TAP2 + CD3D + CTLA4 | 0.953 | 0.887 | 1 |
| 4 days | CD177 + IFNG + S100A9 + CX3CR1 + TAP2 + CTLA4 | 0.941 | 0.861 | 1 |
| 4 days | CD177 + IFNG + S100A9 + TAP2 | 0.75 | 0.561 | 0.939 |
| 4 days | CD177 + IFNG + S100A9 + TAP2 + CD3D | 0.754 | 0.568 | 0.94 |
| 4 days | CD177 + IFNG + S100A9 + TAP2 + CD3D + CTLA4 | 0.762 | 0.582 | 0.942 |
| 4 days | CD177 + IFNG + S100A9 + TAP2 + CTLA4 | 0.758 | 0.575 | 0.94 |
| 4 days | CD177 + IFNG + TAP2 | 0.695 | 0.505 | 0.886 |
| 4 days | CD177 + IFNG + TAP2 + CD3D | 0.691 | 0.5 | 0.883 |
| 4 days | CD177 + IFNG + TAP2 + CD3D + CTLA4 | 0.695 | 0.508 | 0.883 |
| 4 days | CD177 + IFNG + TAP2 + CTLA4 | 0.688 | 0.496 | 0.879 |
| 4 days | CD177 + IL1R2 + C3AR1 + CD3D | 0.691 | 0.501 | 0.882 |
| 4 days | CD177 + IL1R2 + C3AR1 + CD3D + CTLA4 | 0.699 | 0.508 | 0.89 |
| 4 days | CD177 + IL1R2 + C3AR1 + CD74 | 0.723 | 0.541 | 0.904 |
| 4 days | CD177 + IL1R2 + C3AR1 + CD74 + CD3D | 0.75 | 0.579 | 0.921 |
| 4 days | CD177 + IL1R2 + C3AR1 + CD74 + CD3D + CTLA4 | 0.758 | 0.589 | 0.926 |
| 4 days | CD177 + IL1R2 + C3AR1 + CD74 + CIITA | 0.73 | 0.549 | 0.912 |
| 4 days | CD177 + IL1R2 + C3AR1 + CD74 + CIITA + CD3D | 0.738 | 0.565 | 0.912 |
| 4 days | CD177 + IL1R2 + C3AR1 + CD74 + CIITA + CD3D + CTLA4 | 0.742 | 0.569 | 0.915 |
| 4 days | CD177 + IL1R2 + C3AR1 + CD74 + CIITA + CTLA4 | 0.746 | 0.573 | 0.919 |
| 4 days | CD177 + IL1R2 + C3AR1 + CD74 + CIITA + TAP2 | 0.738 | 0.562 | 0.915 |
| 4 days | CD177 + IL1R2 + C3AR1 + CD74 + CIITA + TAP2 + CD3D | 0.742 | 0.568 | 0.916 |
| 4 days | CD177 + IL1R2 + C3AR1 + CD74 + CIITA + TAP2 + CD3D + | 0.754 | 0.585 | 0.922 |
| | CTLA4 | | | |
| 4 days | CD177 + IL1R2 + C3AR1 + CD74 + CIITA + TAP2 + CTLA4 | 0.734 | 0.557 | 0.912 |
| 4 days | CD177 + IL1R2 + C3AR1 + CD74 + CTLA4 | 0.738 | 0.561 | 0.916 |
| 4 days | CD177 + IL1R2 + C3AR1 + CD74 + CX3CR1 | 0.738 | 0.561 | 0.915 |
| 4 days | CD177 + IL1R2 + C3AR1 + CD74 + CX3CR1 + CD3D | 0.758 | 0.588 | 0.927 |
| 4 days | CD177 + IL1R2 + C3AR1 + CD74 + CX3CR1 + CD3D + CTLA4 | 0.758 | 0.586 | 0.929 |
| 4 days | CD177 + IL1R2 + C3AR1 + CD74 + CX3CR1 + CIITA | 0.746 | 0.568 | 0.924 |
| 4 days | CD177 + IL1R2 + C3AR1 + CD74 + CX3CR1 + CIITA + CD3D | 0.762 | 0.593 | 0.931 |
| 4 days | CD177 + IL1R2 + C3AR1 + CD74 + CX3CR1 + CIITA + CD3D + | 0.766 | 0.601 | 0.93 |
| | CTLA4 | | | |
| 4 days | CD177 + IL1R2 + C3AR1 + CD74 + CX3CR1 + CIITA + CTLA4 | 0.738 | 0.561 | 0.916 |
| 4 days | CD177 + IL1R2 + C3AR1 + CD74 + CX3CR1 + CIITA + TAP2 | 0.77 | 0.602 | 0.937 |
| 4 days | CD177 + IL1R2 + C3AR1 + CD74 + CX3CR1 + CIITA + TAP2 + | 0.789 | 0.625 | 0.953 |
| | CD3D | | | |
| 4 days | CD177 + IL1R2 + C3AR1 + CD74 + CX3CR1 + CIITA + TAP2 + | 0.789 | 0.625 | 0.953 |
| | CD3D + CTLA4 | | | |
| 4 days | CD177 + IL1R2 + C3AR1 + CD74 + CX3CR1 + CIITA + TAP2 + | 0.781 | 0.618 | 0.944 |
| | CTLA4 | | | |
| 4 days | CD177 + IL1R2 + C3AR1 + CD74 + CX3CR1 + CTLA4 | 0.75 | 0.573 | 0.927 |
| 4 days | CD177 + IL1R2 + C3AR1 + CD74 + CX3CR1 + TAP2 | 0.777 | 0.611 | 0.944 |
| 4 days | CD177 + IL1R2 + C3AR1 + CD74 + CX3CR1 + TAP2 + CD3D | 0.777 | 0.614 | 0.941 |
| 4 days | CD177 + IL1R2 + C3AR1 + CD74 + CX3CR1 + TAP2 + CD3D + | 0.812 | 0.656 | 0.969 |
| | CTLA4 | | | |
| 4 days | CD177 + IL1R2 + C3AR1 + CD74 + CX3CR1 + TAP2 + CTLA4 | 0.797 | 0.638 | 0.955 |
| 4 days | CD177 + IL1R2 + C3AR1 + CD74 + IFNG | 0.738 | 0.563 | 0.914 |
| 4 days | CD177 + IL1R2 + C3AR1 + CD74 + IFNG + CD3D | 0.75 | 0.577 | 0.923 |
| 4 days | CD177 + IL1R2 + C3AR1 + CD74 + IFNG + CD3D + CTLA4 | 0.762 | 0.594 | 0.929 |
| 4 days | CD177 + IL1R2 + C3AR1 + CD74 + IFNG + CIITA | 0.738 | 0.563 | 0.913 |
| 4 days | CD177 + IL1R2 + C3AR1 + CD74 + IFNG + CIITA + CD3D | 0.75 | 0.578 | 0.922 |
| 4 days | CD177 + IL1R2 + C3AR1 + CD74 + IFNG + CIITA + CD3D + | 0.758 | 0.584 | 0.932 |
| | CTLA4 | | | |
| 4 days | CD177 + IL1R2 + C3AR1 + CD74 + IFNG + CIITA + CTLA4 | 0.754 | 0.58 | 0.928 |
| 4 days | CD177 + IL1R2 + C3AR1 + CD74 + IFNG + CIITA + TAP2 | 0.742 | 0.568 | 0.917 |
| 4 days | CD177 + IL1R2 + C3AR1 + CD74 + IFNG + CIITA + TAP2 + | 0.754 | 0.584 | 0.924 |
| | CD3D | | | |
| 4 days | CD177 + IL1R2 + C3AR1 + CD74 + IFNG + CIITA + TAP2 + | 0.758 | 0.584 | 0.932 |
| | CD3D + CTLA4 | | | |
| 4 days | CD177 + IL1R2 + C3AR1 + CD74 + IFNG + CIITA + TAP2 + | 0.754 | 0.58 | 0.928 |
| | CTLA4 | | | |
| 4 days | CD177 + IL1R2 + C3AR1 + CD74 + IFNG + CTLA4 | 0.758 | 0.589 | 0.927 |
| 4 days | CD177 + IL1R2 + C3AR1 + CD74 + IFNG + CX3CR1 | 0.867 | 0.737 | 0.997 |
| 4 days | CD177 + IL1R2 + C3AR1 + CD74 + IFNG + CX3CR1 + CD3D | 0.871 | 0.752 | 0.99 |
| 4 days | CD177 + IL1R2 + C3AR1 + CD74 + IFNG + CX3CR1 + CD3D + | 0.914 | 0.821 | 1 |
| | CTLA4 | | | |
| 4 days | CD177 + IL1R2 + C3AR1 + CD74 + IFNG + CX3CR1 + CIITA | 0.875 | 0.753 | 0.997 |
| 4 days | CD177 + IL1R2 + C3AR1 + CD74 + IFNG + CX3CR1 + CIITA + | 0.871 | 0.753 | 0.989 |
| | CD3D | | | |
| 4 days | CD177 + IL1R2 + C3AR1 + CD74 + IFNG + CX3CR1 + CIITA + | 0.918 | 0.828 | 1 |
| | CD3D + CTLA4 | | | |
| 4 days | CD177 + IL1R2 + C3AR1 + CD74 + IFNG + CX3CR1 + CIITA + | 0.914 | 0.82 | 1 |
| | CTLA4 | | | |
| 4 days | CD177 + IL1R2 + C3AR1 + CD74 + IFNG + CX3CR1 + CIITA + | 0.855 | 0.725 | 0.986 |
| | TAP2 | | | |
| 4 days | CD177 + IL1R2 + C3AR1 + CD74 + IFNG + CX3CR1 + CIITA + | 0.871 | 0.752 | 0.99 |
| | TAP2 + CD3D | | | |
| 4 days | CD177 + IL1R2 + C3AR1 + CD74 + IFNG + CX3CR1 + CIITA + | 0.891 | 0.782 | 1 |
| | TAP2 + CD3D + CTLA4 | | | |
| 4 days | CD177 + IL1R2 + C3AR1 + CD74 + IFNG + CX3CR1 + CIITA + | 0.895 | 0.788 | 1 |
| | TAP2 + CTLA4 | | | |
| 4 days | CD177 + IL1R2 + C3AR1 + CD74 + IFNG + CX3CR1 + CTLA4 | 0.902 | 0.801 | 1 |
| 4 days | CD177 + IL1R2 + C3AR1 + CD74 + IFNG + CX3CR1 + TAP2 | 0.863 | 0.738 | 0.989 |
| 4 days | CD177 + IL1R2 + C3AR1 + CD74 + IFNG + CX3CR1 + TAP2 + | 0.887 | 0.777 | 0.997 |
| | CD3D | | | |
| 4 days | CD177 + IL1R2 + C3AR1 + CD74 + IFNG + CX3CR1 + TAP2 + | 0.891 | 0.78 | 1 |
| | CD3D + CTLA4 | | | |
| 4 days | CD177 + IL1R2 + C3AR1 + CD74 + IFNG + CX3CR1 + TAP2 + | 0.891 | 0.78 | 1 |
| | CTLA4 | | | |
| 4 days | CD177 + IL1R2 + C3AR1 + CD74 + IFNG + S100A9 | 0.828 | 0.683 | 0.973 |
| 4 days | CD177 + IL1R2 + C3AR1 + CD74 + IFNG + S100A9 + CD3D | 0.836 | 0.697 | 0.975 |
| 4 days | CD177 + IL1R2 + C3AR1 + CD74 + IFNG + S100A9 + CD3D + | 0.844 | 0.71 | 0.977 |
| | CTLA4 | | | |
| 4 days | CD177 + IL1R2 + C3AR1 + CD74 + IFNG + S100A9 + CIITA | 0.824 | 0.673 | 0.975 |
| 4 days | CD177 + IL1R2 + C3AR1 + CD74 + IFNG + S100A9 + CIITA + | 0.844 | 0.71 | 0.978 |
| | CD3D | | | |
| 4 days | CD177 + IL1R2 + C3AR1 + CD74 + IFNG + S100A9 + CIITA + | 0.84 | 0.705 | 0.975 |
| | CD3D + CTLA4 | | | |
| 4 days | CD177 + IL1R2 + C3AR1 + CD74 + IFNG + S100A9 + CIITA + | 0.82 | 0.675 | 0.966 |
| | CTLA4 | | | |
| 4 days | CD177 + IL1R2 + C3AR1 + CD74 + IFNG + S100A9 + CIITA + | 0.855 | 0.718 | 0.993 |
| | TAP2 | | | |
| 4 days | CD177 + IL1R2 + C3AR1 + CD74 + IFNG + S100A9 + CIITA + | 0.855 | 0.726 | 0.985 |
| | TAP2 + CD3D | | | |
| 4 days | CD177 + IL1R2 + C3AR1 + CD74 + IFNG + S100A9 + CIITA + | 0.848 | 0.713 | 0.982 |
| | TAP2 + CD3D + CTLA4 | | | |
| 4 days | CD177 + IL1R2 + C3AR1 + CD74 + IFNG + S100A9 + CIITA + | 0.84 | 0.701 | 0.979 |
| | TAP2 + CTLA4 | | | |
| 4 days | CD177 + IL1R2 + C3AR1 + CD74 + IFNG + S100A9 + CTLA4 | 0.828 | 0.688 | 0.969 |
| 4 days | CD177 + IL1R2 + C3AR1 + CD74 + IFNG + S100A9 + CX3CR1 | 0.941 | 0.864 | 1 |
| 4 days | CD177 + IL1R2 + C3AR1 + CD74 + IFNG + S100A9 + CX3CR1 + | 0.977 | 0.928 | 1 |
| | CD3D | | | |
| 4 days | CD177 + IL1R2 + C3AR1 + CD74 + IFNG + S100A9 + CX3CR1 + | 0.945 | 0.871 | 1 |
| | CIITA | | | |
| 4 days | CD177 + IL1R2 + C3AR1 + CD74 + IFNG + S100A9 + CX3CR1 + | 0.984 | 0.951 | 1 |
| | CIITA + CD3D | | | |
| 4 days | CD177 + IL1R2 + C3AR1 + CD74 + IFNG + S100A9 + CX3CR1 + | 0.945 | 0.871 | 1 |
| | CIITA + TAP2 | | | |
| 4 days | CD177 + IL1R2 + C3AR1 + CD74 + IFNG + S100A9 + CX3CR1 + | 0.941 | 0.864 | 1 |
| | TAP2 | | | |
| 4 days | CD177 + IL1R2 + C3AR1 + CD74 + IFNG + S100A9 + TAP2 | 0.84 | 0.7 | 0.98 |
| 4 days | CD177 + IL1R2 + C3AR1 + CD74 + IFNG + S100A9 + TAP2 + | 0.848 | 0.714 | 0.981 |
| | CD3D | | | |
| 4 days | CD177 + IL1R2 + C3AR1 + CD74 + IFNG + S100A9 + TAP2 + | 0.844 | 0.709 | 0.979 |
| | CD3D + CTLA4 | | | |
| 4 days | CD177 + IL1R2 + C3AR1 + CD74 + IFNG + S100A9 + TAP2 + | 0.824 | 0.68 | 0.969 |
| | CTLA4 | | | |
| 4 days | CD177 + IL1R2 + C3AR1 + CD74 + IFNG + TAP2 | 0.738 | 0.563 | 0.914 |
| 4 days | CD177 + IL1R2 + C3AR1 + CD74 + IFNG + TAP2 + CD3D | 0.746 | 0.572 | 0.92 |
| 4 days | CD177 + IL1R2 + C3AR1 + CD74 + IFNG + TAP2 + CD3D + | 0.762 | 0.593 | 0.93 |
| | CTLA4 | | | |
| 4 days | CD177 + IL1R2 + C3AR1 + CD74 + IFNG + TAP2 + CTLA4 | 0.758 | 0.589 | 0.927 |
| 4 days | CD177 + IL1R2 + C3AR1 + CD74 + S100A9 | 0.828 | 0.674 | 0.982 |
| 4 days | CD177 + IL1R2 + C3AR1 + CD74 + S100A9 + CD3D | 0.828 | 0.683 | 0.973 |
| 4 days | CD177 + IL1R2 + C3AR1 + CD74 + S100A9 + CD3D + CTLA4 | 0.828 | 0.683 | 0.973 |
| 4 days | CD177 + IL1R2 + C3AR1 + CD74 + S100A9 + CIITA | 0.828 | 0.676 | 0.98 |
| 4 days | CD177 + IL1R2 + C3AR1 + CD74 + S100A9 + CIITA + CD3D | 0.824 | 0.676 | 0.972 |
| 4 days | CD177 + IL1R2 + C3AR1 + CD74 + S100A9 + CIITA + CD3D + | 0.824 | 0.676 | 0.972 |
| | CTLA4 | | | |
| 4 days | CD177 + IL1R2 + C3AR1 + CD74 + S100A9 + CIITA + CTLA4 | 0.828 | 0.677 | 0.979 |
| 4 days | CD177 + IL1R2 + C3AR1 + CD74 + S100A9 + CIITA + TAP2 | 0.828 | 0.674 | 0.982 |
| 4 days | CD177 + IL1R2 + C3AR1 + CD74 + S100A9 + CIITA + TAP2 + | 0.836 | 0.694 | 0.978 |
| | CD3D | | | |
| 4 days | CD177 + IL1R2 + C3AR1 + CD74 + S100A9 + CIITA + TAP2 + | 0.844 | 0.705 | 0.983 |
| | CD3D + CTLA4 | | | |
| 4 days | CD177 + IL1R2 + C3AR1 + CD74 + S100A9 + CIITA + TAP2 + | 0.828 | 0.677 | 0.979 |
| | CTLA4 | | | |
| 4 days | CD177 + IL1R2 + C3AR1 + CD74 + S100A9 + CTLA4 | 0.828 | 0.677 | 0.979 |
| 4 days | CD177 + IL1R2 + C3AR1 + CD74 + S100A9 + CX3CR1 | 0.859 | 0.713 | 1 |
| 4 days | CD177 + IL1R2 + C3AR1 + CD74 + S100A9 + CX3CR1 + CD3D | 0.891 | 0.766 | 1 |
| 4 days | CD177 + IL1R2 + C3AR1 + CD74 + S100A9 + CX3CR1 + CD3D + | 0.895 | 0.775 | 1 |
| | CTLA4 | | | |
| 4 days | CD177 + IL1R2 + C3AR1 + CD74 + S100A9 + CX3CR1 + CIITA | 0.867 | 0.734 | 1 |
| 4 days | CD177 + IL1R2 + C3AR1 + CD74 + S100A9 + CX3CR1 + CIITA + | 0.902 | 0.788 | 1 |
| | CD3D | | | |
| 4 days | CD177 + IL1R2 + C3AR1 + CD74 + S100A9 + CX3CR1 + CIITA + | 0.895 | 0.782 | 1 |
| | CD3D + CTLA4 | | | |
| 4 days | CD177 + IL1R2 + C3AR1 + CD74 + S100A9 + CX3CR1 + CIITA + | 0.871 | 0.74 | 1 |
| | CTLA4 | | | |
| 4 days | CD177 + IL1R2 + C3AR1 + CD74 + S100A9 + CX3CR1 + CIITA + | 0.887 | 0.762 | 1 |
| | TAP2 | | | |
| 4 days | CD177 + IL1R2 + C3AR1 + CD74 + S100A9 + CX3CR1 + CIITA + | 0.902 | 0.792 | 1 |
| | TAP2 + CD3D | | | |
| 4 days | CD177 + IL1R2 + C3AR1 + CD74 + S100A9 + CX3CR1 + CIITA + | 0.906 | 0.804 | 1 |
| | TAP2 + CD3D + CTLA4 | | | |
| 4 days | CD177 + IL1R2 + C3AR1 + CD74 + S100A9 + CX3CR1 + CIITA + | 0.887 | 0.759 | 1 |
| | TAP2 + CTLA4 | | | |
| 4 days | CD177 + IL1R2 + C3AR1 + CD74 + S100A9 + CX3CR1 + CTLA4 | 0.863 | 0.722 | 1 |
| 4 days | CD177 + IL1R2 + C3AR1 + CD74 + S100A9 + CX3CR1 + TAP2 | 0.871 | 0.735 | 1 |
| 4 days | CD177 + IL1R2 + C3AR1 + CD74 + S100A9 + CX3CR1 + TAP2 + | 0.898 | 0.781 | 1 |
| | CD3D | | | |
| 4 days | CD177 + IL1R2 + C3AR1 + CD74 + S100A9 + CX3CR1 + TAP2 + | 0.891 | 0.772 | 1 |
| | CD3D + CTLA4 | | | |
| 4 days | CD177 + IL1R2 + C3AR1 + CD74 + S100A9 + CX3CR1 + TAP2 + | 0.875 | 0.737 | 1 |
| | CTLA4 | | | |
| 4 days | CD177 + IL1R2 + C3AR1 + CD74 + S100A9 + TAP2 | 0.824 | 0.669 | 0.979 |
| 4 days | CD177 + IL1R2 + C3AR1 + CD74 + S100A9 + TAP2 + CD3D | 0.836 | 0.694 | 0.978 |
| 4 days | CD177 + IL1R2 + C3AR1 + CD74 + S100A9 + TAP2 + CD3D + | 0.848 | 0.712 | 0.983 |
| | CTLA4 | | | |
| 4 days | CD177 + IL1R2 + C3AR1 + CD74 + S100A9 + TAP2 + CTLA4 | 0.828 | 0.677 | 0.979 |
| 4 days | CD177 + IL1R2 + C3AR1 + CD74 + TAP2 | 0.727 | 0.544 | 0.909 |
| 4 days | CD177 + IL1R2 + C3AR1 + CD74 + TAP2 + CD3D | 0.746 | 0.573 | 0.92 |
| 4 days | CD177 + IL1R2 + C3AR1 + CD74 + TAP2 + CD3D + CTLA4 | 0.734 | 0.556 | 0.913 |
| 4 days | CD177 + IL1R2 + C3AR1 + CD74 + TAP2 + CTLA4 | 0.738 | 0.56 | 0.916 |
| 4 days | CD177 + IL1R2 + C3AR1 + CIITA | 0.734 | 0.554 | 0.915 |
| 4 days | CD177 + IL1R2 + C3AR1 + CIITA + CD3D | 0.734 | 0.551 | 0.918 |
| 4 days | CD177 + IL1R2 + C3AR1 + CIITA + CD3D + CTLA4 | 0.719 | 0.535 | 0.902 |
| 4 days | CD177 + IL1R2 + C3AR1 + CIITA + CTLA4 | 0.703 | 0.515 | 0.891 |
| 4 days | CD177 + IL1R2 + C3AR1 + CIITA + TAP2 | 0.715 | 0.525 | 0.905 |
| 4 days | CD177 + IL1R2 + C3AR1 + CIITA + TAP2 + CD3D | 0.742 | 0.561 | 0.924 |
| 4 days | CD177 + IL1R2 + C3AR1 + CIITA + TAP2 + CD3D + CTLA4 | 0.723 | 0.539 | 0.907 |
| 4 days | CD177 + IL1R2 + C3AR1 + CIITA + TAP2 + CTLA4 | 0.727 | 0.544 | 0.91 |
| 4 days | CD177 + IL1R2 + C3AR1 + CTLA4 | 0.691 | 0.499 | 0.884 |
| 4 days | CD177 + IL1R2 + C3AR1 + CX3CR1 | 0.691 | 0.501 | 0.881 |
| 4 days | CD177 + IL1R2 + C3AR1 + CX3CR1 + CD3D | 0.699 | 0.51 | 0.888 |
| 4 days | CD177 + IL1R2 + C3AR1 + CX3CR1 + CD3D + CTLA4 | 0.688 | 0.493 | 0.882 |
| 4 days | CD177 + IL1R2 + C3AR1 + CX3CR1 + CIITA | 0.77 | 0.597 | 0.942 |
| 4 days | CD177 + IL1R2 + C3AR1 + CX3CR1 + CIITA + CD3D | 0.715 | 0.519 | 0.911 |
| 4 days | CD177 + IL1R2 + C3AR1 + CX3CR1 + CIITA + CD3D + CTLA4 | 0.715 | 0.519 | 0.911 |
| 4 days | CD177 + IL1R2 + C3AR1 + CX3CR1 + CIITA + CTLA4 | 0.711 | 0.52 | 0.902 |
| 4 days | CD177 + IL1R2 + C3AR1 + CX3CR1 + CIITA + TAP2 | 0.777 | 0.615 | 0.939 |
| 4 days | CD177 + IL1R2 + C3AR1 + CX3CR1 + CIITA + TAP2 + CD3D | 0.789 | 0.629 | 0.949 |
| 4 days | CD177 + IL1R2 + C3AR1 + CX3CR1 + CIITA + TAP2 + CD3D + | 0.777 | 0.612 | 0.942 |
| | CTLA4 | | | |
| 4 days | CD177 + IL1R2 + C3AR1 + CX3CR1 + CIITA + TAP2 + CTLA4 | 0.781 | 0.62 | 0.943 |
| 4 days | CD177 + IL1R2 + C3AR1 + CX3CR1 + CTLA4 | 0.711 | 0.522 | 0.9 |
| 4 days | CD177 + IL1R2 + C3AR1 + CX3CR1 + TAP2 | 0.727 | 0.547 | 0.906 |
| 4 days | CD177 + IL1R2 + C3AR1 + CX3CR1 + TAP2 + CD3D | 0.734 | 0.559 | 0.909 |
| 4 days | CD177 + IL1R2 + C3AR1 + CX3CR1 + TAP2 + CD3D + CTLA4 | 0.734 | 0.56 | 0.908 |
| 4 days | CD177 + IL1R2 + C3AR1 + CX3CR1 + TAP2 + CTLA4 | 0.734 | 0.56 | 0.908 |
| 4 days | CD177 + IL1R2 + C3AR1 + IFNG | 0.73 | 0.548 | 0.913 |
| 4 days | CD177 + IL1R2 + C3AR1 + IFNG + CD3D | 0.754 | 0.577 | 0.931 |
| 4 days | CD177 + IL1R2 + C3AR1 + IFNG + CD3D + CTLA4 | 0.75 | 0.579 | 0.921 |
| 4 days | CD177 + IL1R2 + C3AR1 + IFNG + CIITA | 0.734 | 0.556 | 0.913 |
| 4 days | CD177 + IL1R2 + C3AR1 + IFNG + CIITA + CD3D | 0.746 | 0.567 | 0.925 |
| 4 days | CD177 + IL1R2 + C3AR1 + IFNG + CIITA + CD3D + CTLA4 | 0.75 | 0.579 | 0.921 |
| 4 days | CD177 + IL1R2 + C3AR1 + IFNG + CIITA + CTLA4 | 0.75 | 0.58 | 0.92 |
| 4 days | CD177 + IL1R2 + C3AR1 + IFNG + CIITA + TAP2 | 0.746 | 0.568 | 0.924 |
| 4 days | CD177 + IL1R2 + C3AR1 + IFNG + CIITA + TAP2 + CD3D | 0.75 | 0.574 | 0.926 |
| 4 days | CD177 + IL1R2 + C3AR1 + IFNG + CIITA + TAP2 + CD3D + | 0.75 | 0.578 | 0.922 |
| | CTLA4 | | | |
| 4 days | CD177 + IL1R2 + C3AR1 + IFNG + CIITA + TAP2 + CTLA4 | 0.766 | 0.597 | 0.934 |
| 4 days | CD177 + IL1R2 + C3AR1 + IFNG + CTLA4 | 0.75 | 0.58 | 0.92 |
| 4 days | CD177 + IL1R2 + C3AR1 + IFNG + CX3CR1 | 0.844 | 0.699 | 0.988 |
| 4 days | CD177 + IL1R2 + C3AR1 + IFNG + CX3CR1 + CD3D | 0.863 | 0.74 | 0.987 |
| 4 days | CD177 + IL1R2 + C3AR1 + IFNG + CX3CR1 + CD3D + CTLA4 | 0.895 | 0.784 | 1 |
| 4 days | CD177 + IL1R2 + C3AR1 + IFNG + CX3CR1 + CIITA | 0.863 | 0.727 | 1 |
| 4 days | CD177 + IL1R2 + C3AR1 + IFNG + CX3CR1 + CIITA + CD3D | 0.875 | 0.754 | 0.996 |
| 4 days | CD177 + IL1R2 + C3AR1 + IFNG + CX3CR1 + CIITA + CD3D + | 0.891 | 0.778 | 1 |
| | CTLA4 | | | |
| 4 days | CD177 + IL1R2 + C3AR1 + IFNG + CX3CR1 + CIITA + CTLA4 | 0.891 | 0.778 | 1 |
| 4 days | CD177 + IL1R2 + C3AR1 + IFNG + CX3CR1 + CIITA + TAP2 | 0.871 | 0.746 | 0.996 |
| 4 days | CD177 + IL1R2 + C3AR1 + IFNG + CX3CR1 + CIITA + TAP2 + | 0.871 | 0.749 | 0.994 |
| | CD3D | | | |
| 4 days | CD177 + IL1R2 + C3AR1 + IFNG + CX3CR1 + CIITA + TAP2 + | 0.895 | 0.779 | 1 |
| | CD3D + CTLA4 | | | |
| 4 days | CD177 + IL1R2 + C3AR1 + IFNG + CX3CR1 + CIITA + TAP2 + | 0.895 | 0.781 | 1 |
| | CTLA4 | | | |
| 4 days | CD177 + IL1R2 + C3AR1 + IFNG + CX3CR1 + CTLA4 | 0.895 | 0.784 | 1 |
| 4 days | CD177 + IL1R2 + C3AR1 + IFNG + CX3CR1 + TAP2 | 0.852 | 0.72 | 0.983 |
| 4 days | CD177 + IL1R2 + C3AR1 + IFNG + CX3CR1 + TAP2 + CD3D | 0.871 | 0.749 | 0.994 |
| 4 days | CD177 + IL1R2 + C3AR1 + IFNG + CX3CR1 + TAP2 + CD3D + | 0.895 | 0.779 | 1 |
| | CTLA4 | | | |
| 4 days | CD177 + IL1R2 + C3AR1 + IFNG + CX3CR1 + TAP2 + CTLA4 | 0.906 | 0.797 | 1 |
| 4 days | CD177 + IL1R2 + C3AR1 + IFNG + S100A9 | 0.828 | 0.681 | 0.976 |
| 4 days | CD177 + IL1R2 + C3AR1 + IFNG + S100A9 + CD3D | 0.836 | 0.698 | 0.974 |
| 4 days | CD177 + IL1R2 + C3AR1 + IFNG + S100A9 + CD3D + CTLA4 | 0.836 | 0.699 | 0.973 |
| 4 days | CD177 + IL1R2 + C3AR1 + IFNG + S100A9 + CIITA | 0.82 | 0.668 | 0.972 |
| 4 days | CD177 + IL1R2 + C3AR1 + IFNG + S100A9 + CIITA + CD3D | 0.844 | 0.71 | 0.978 |
| 4 days | CD177 + IL1R2 + C3AR1 + IFNG + S100A9 + CIITA + CD3D + | 0.84 | 0.705 | 0.975 |
| | CTLA4 | | | |
| 4 days | CD177 + IL1R2 + C3AR1 + IFNG + S100A9 + CIITA + CTLA4 | 0.82 | 0.675 | 0.966 |
| 4 days | CD177 + IL1R2 + C3AR1 + IFNG + S100A9 + CIITA + TAP2 | 0.852 | 0.713 | 0.99 |
| 4 days | CD177 + IL1R2 + C3AR1 + IFNG + S100A9 + CIITA + TAP2 + | 0.855 | 0.726 | 0.985 |
| | CD3D | | | |
| 4 days | CD177 + IL1R2 + C3AR1 + IFNG + S100A9 + CIITA + TAP2 + | 0.848 | 0.713 | 0.982 |
| | CD3D + CTLA4 | | | |
| 4 days | CD177 + IL1R2 + C3AR1 + IFNG + S100A9 + CIITA + TAP2 + | 0.84 | 0.701 | 0.979 |
| | CTLA4 | | | |
| 4 days | CD177 + IL1R2 + C3AR1 + IFNG + S100A9 + CTLA4 | 0.824 | 0.681 | 0.968 |
| 4 days | CD177 + IL1R2 + C3AR1 + IFNG + S100A9 + CX3CR1 | 0.941 | 0.864 | 1 |
| 4 days | CD177 + IL1R2 + C3AR1 + IFNG + S100A9 + CX3CR1 + CD3D | 0.977 | 0.928 | 1 |
| 4 days | CD177 + IL1R2 + C3AR1 + IFNG + S100A9 + CX3CR1 + CIITA | 0.938 | 0.857 | 1 |
| 4 days | CD177 + IL1R2 + C3AR1 + IFNG + S100A9 + CX3CR1 + CIITA + | 0.98 | 0.939 | 1 |
| | CD3D | | | |
| 4 days | CD177 + IL1R2 + C3AR1 + IFNG + S100A9 + CX3CR1 + CIITA + | 0.938 | 0.857 | 1 |
| | TAP2 | | | |
| 4 days | CD177 + IL1R2 + C3AR1 + IFNG + S100A9 + CX3CR1 + TAP2 | 0.941 | 0.864 | 1 |
| 4 days | CD177 + IL1R2 + C3AR1 + IFNG + S100A9 + CX3CR1 + TAP2 + | 0.984 | 0.951 | 1 |
| | CD3D | | | |
| 4 days | CD177 + IL1R2 + C3AR1 + IFNG + S100A9 + TAP2 | 0.84 | 0.696 | 0.984 |
| 4 days | CD177 + IL1R2 + C3AR1 + IFNG + S100A9 + TAP2 + CD3D | 0.84 | 0.703 | 0.976 |
| 4 days | CD177 + IL1R2 + C3AR1 + IFNG + S100A9 + TAP2 + CD3D + | 0.84 | 0.703 | 0.976 |
| | CTLA4 | | | |
| 4 days | CD177 + IL1R2 + C3AR1 + IFNG + S100A9 + TAP2 + CTLA4 | 0.824 | 0.68 | 0.969 |
| 4 days | CD177 + IL1R2 + C3AR1 + IFNG + TAP2 | 0.742 | 0.562 | 0.923 |
| 4 days | CD177 + IL1R2 + C3AR1 + IFNG + TAP2 + CD3D | 0.75 | 0.571 | 0.929 |
| 4 days | CD177 + IL1R2 + C3AR1 + IFNG + TAP2 + CD3D + CTLA4 | 0.758 | 0.588 | 0.928 |
| 4 days | CD177 + IL1R2 + C3AR1 + IFNG + TAP2 + CTLA4 | 0.766 | 0.597 | 0.934 |
| 4 days | CD177 + IL1R2 + C3AR1 + S100A9 | 0.82 | 0.663 | 0.978 |
| 4 days | CD177 + IL1R2 + C3AR1 + S100A9 + CD3D | 0.824 | 0.676 | 0.972 |
| 4 days | CD177 + IL1R2 + C3AR1 + S100A9 + CD3D + CTLA4 | 0.82 | 0.67 | 0.971 |
| 4 days | CD177 + IL1R2 + C3AR1 + S100A9 + CIITA | 0.816 | 0.658 | 0.975 |
| 4 days | CD177 + IL1R2 + C3AR1 + S100A9 + CIITA + CD3D | 0.832 | 0.686 | 0.978 |
| 4 days | CD177 + IL1R2 + C3AR1 + S100A9 + CIITA + CD3D + CTLA4 | 0.836 | 0.692 | 0.979 |
| 4 days | CD177 + IL1R2 + C3AR1 + S100A9 + CIITA + CTLA4 | 0.828 | 0.677 | 0.979 |
| 4 days | CD177 + IL1R2 + C3AR1 + S100A9 + CIITA + TAP2 | 0.832 | 0.679 | 0.985 |
| 4 days | CD177 + IL1R2 + C3AR1 + S100A9 + CIITA + TAP2 + CD3D | 0.832 | 0.688 | 0.976 |
| 4 days | CD177 + IL1R2 + C3AR1 + S100A9 + CIITA + TAP2 + CD3D + | 0.844 | 0.705 | 0.983 |
| | CTLA4 | | | |
| 4 days | CD177 + IL1R2 + C3AR1 + S100A9 + CIITA + TAP2 + CTLA4 | 0.828 | 0.677 | 0.979 |
| 4 days | CD177 + IL1R2 + C3AR1 + S100A9 + CTLA4 | 0.828 | 0.677 | 0.979 |
| 4 days | CD177 + IL1R2 + C3AR1 + S100A9 + CX3CR1 | 0.859 | 0.716 | 1 |
| 4 days | CD177 + IL1R2 + C3AR1 + S100A9 + CX3CR1 + CD3D | 0.891 | 0.766 | 1 |
| 4 days | CD177 + IL1R2 + C3AR1 + S100A9 + CX3CR1 + CD3D + CTLA4 | 0.887 | 0.764 | 1 |
| 4 days | CD177 + IL1R2 + C3AR1 + S100A9 + CX3CR1 + CIITA | 0.867 | 0.731 | 1 |
| 4 days | CD177 + IL1R2 + C3AR1 + S100A9 + CX3CR1 + CIITA + CD3D | 0.895 | 0.774 | 1 |
| 4 days | CD177 + IL1R2 + C3AR1 + S100A9 + CX3CR1 + CIITA + CD3D + | 0.895 | 0.779 | 1 |
| | CTLA4 | | | |
| 4 days | CD177 + IL1R2 + C3AR1 + S100A9 + CX3CR1 + CIITA + CTLA4 | 0.867 | 0.728 | 1 |
| 4 days | CD177 + IL1R2 + C3AR1 + S100A9 + CX3CR1 + CIITA + TAP2 | 0.883 | 0.751 | 1 |
| 4 days | CD177 + IL1R2 + C3AR1 + S100A9 + CX3CR1 + CIITA + TAP2 + | 0.906 | 0.796 | 1 |
| | CD3D | | | |
| 4 days | CD177 + IL1R2 + C3AR1 + S100A9 + CX3CR1 + CIITA + TAP2 + | 0.898 | 0.79 | 1 |
| | CD3D + CTLA4 | | | |
| 4 days | CD177 + IL1R2 + C3AR1 + S100A9 + CX3CR1 + CIITA + TAP2 + | 0.883 | 0.751 | 1 |
| | CTLA4 | | | |
| 4 days | CD177 + IL1R2 + C3AR1 + S100A9 + CX3CR1 + CTLA4 | 0.863 | 0.722 | 1 |
| 4 days | CD177 + IL1R2 + C3AR1 + S100A9 + CX3CR1 + TAP2 | 0.871 | 0.735 | 1 |
| 4 days | CD177 + IL1R2 + C3AR1 + S100A9 + CX3CR1 + TAP2 + CD3D | 0.898 | 0.781 | 1 |
| 4 days | CD177 + IL1R2 + C3AR1 + S100A9 + CX3CR1 + TAP2 + CD3D + | 0.895 | 0.782 | 1 |
| | CTLA4 | | | |
| 4 days | CD177 + IL1R2 + C3AR1 + S100A9 + CX3CR1 + TAP2 + CTLA4 | 0.875 | 0.737 | 1 |
| 4 days | CD177 + IL1R2 + C3AR1 + S100A9 + TAP2 | 0.832 | 0.679 | 0.985 |
| 4 days | CD177 + IL1R2 + C3AR1 + S100A9 + TAP2 + CD3D | 0.832 | 0.69 | 0.975 |
| 4 days | CD177 + IL1R2 + C3AR1 + S100A9 + TAP2 + CD3D + CTLA4 | 0.848 | 0.712 | 0.983 |
| 4 days | CD177 + IL1R2 + C3AR1 + S100A9 + TAP2 + CTLA4 | 0.82 | 0.667 | 0.974 |
| 4 days | CD177 + IL1R2 + C3AR1 + TAP2 | 0.707 | 0.512 | 0.902 |
| 4 days | CD177 + IL1R2 + C3AR1 + TAP2 + CD3D | 0.707 | 0.513 | 0.902 |
| 4 days | CD177 + IL1R2 + C3AR1 + TAP2 + CD3D + CTLA4 | 0.73 | 0.551 | 0.91 |
| 4 days | CD177 + IL1R2 + C3AR1 + TAP2 + CTLA4 | 0.73 | 0.552 | 0.909 |
| 4 days | CD177 + IL1R2 + CD3D + CTLA4 | 0.691 | 0.496 | 0.887 |
| 4 days | CD177 + IL1R2 + CD74 | 0.715 | 0.531 | 0.898 |
| 4 days | CD177 + IL1R2 + CD74 + CD3D | 0.75 | 0.579 | 0.921 |
| 4 days | CD177 + IL1R2 + CD74 + CD3D + CTLA4 | 0.746 | 0.573 | 0.919 |
| 4 days | CD177 + IL1R2 + CD74 + CIITA | 0.742 | 0.569 | 0.916 |
| 4 days | CD177 + IL1R2 + CD74 + CIITA + CD3D | 0.738 | 0.564 | 0.913 |
| 4 days | CD177 + IL1R2 + CD74 + CIITA + CD3D + CTLA4 | 0.746 | 0.575 | 0.917 |
| 4 days | CD177 + IL1R2 + CD74 + CIITA + CTLA4 | 0.742 | 0.569 | 0.915 |
| 4 days | CD177 + IL1R2 + CD74 + CIITA + TAP2 | 0.738 | 0.564 | 0.913 |
| 4 days | CD177 + IL1R2 + CD74 + CIITA + TAP2 + CD3D | 0.746 | 0.573 | 0.919 |
| 4 days | CD177 + IL1R2 + CD74 + CIITA + TAP2 + CD3D + CTLA4 | 0.75 | 0.577 | 0.923 |
| 4 days | CD177 + IL1R2 + CD74 + CIITA + TAP2 + CTLA4 | 0.746 | 0.571 | 0.921 |
| 4 days | CD177 + IL1R2 + CD74 + CTLA4 | 0.738 | 0.561 | 0.916 |
| 4 days | CD177 + IL1R2 + CD74 + CX3CR1 | 0.742 | 0.565 | 0.92 |
| 4 days | CD177 + IL1R2 + CD74 + CX3CR1 + CD3D | 0.766 | 0.601 | 0.931 |
| 4 days | CD177 + IL1R2 + CD74 + CX3CR1 + CD3D + CTLA4 | 0.75 | 0.578 | 0.922 |
| 4 days | CD177 + IL1R2 + CD74 + CX3CR1 + CIITA | 0.742 | 0.563 | 0.921 |
| 4 days | CD177 + IL1R2 + CD74 + CX3CR1 + CIITA + CD3D | 0.723 | 0.539 | 0.906 |
| 4 days | CD177 + IL1R2 + CD74 + CX3CR1 + CIITA + CD3D + CTLA4 | 0.738 | 0.562 | 0.914 |
| 4 days | CD177 + IL1R2 + CD74 + CX3CR1 + CIITA + CTLA4 | 0.738 | 0.561 | 0.916 |
| 4 days | CD177 + IL1R2 + CD74 + CX3CR1 + CIITA + TAP2 | 0.77 | 0.602 | 0.937 |
| 4 days | CD177 + IL1R2 + CD74 + CX3CR1 + CIITA + TAP2 + CD3D | 0.77 | 0.601 | 0.938 |
| 4 days | CD177 + IL1R2 + CD74 + CX3CR1 + CIITA + TAP2 + CD3D + | 0.77 | 0.601 | 0.939 |
| | CTLA4 | | | |
| 4 days | CD177 + IL1R2 + CD74 + CX3CR1 + CIITA + TAP2 + CTLA4 | 0.785 | 0.624 | 0.947 |
| 4 days | CD177 + IL1R2 + CD74 + CX3CR1 + CTLA4 | 0.75 | 0.573 | 0.927 |
| 4 days | CD177 + IL1R2 + CD74 + CX3CR1 + TAP2 | 0.781 | 0.617 | 0.945 |
| 4 days | CD177 + IL1R2 + CD74 + CX3CR1 + TAP2 + CD3D | 0.77 | 0.604 | 0.935 |
| 4 days | CD177 + IL1R2 + CD74 + CX3CR1 + TAP2 + CD3D + CTLA4 | 0.805 | 0.652 | 0.958 |
| 4 days | CD177 + IL1R2 + CD74 + CX3CR1 + TAP2 + CTLA4 | 0.793 | 0.633 | 0.952 |
| 4 days | CD177 + IL1R2 + CD74 + IFNG | 0.742 | 0.57 | 0.915 |
| 4 days | CD177 + IL1R2 + CD74 + IFNG + CD3D | 0.758 | 0.591 | 0.925 |
| 4 days | CD177 + IL1R2 + CD74 + IFNG + CD3D + CTLA4 | 0.754 | 0.583 | 0.925 |
| 4 days | CD177 + IL1R2 + CD74 + IFNG + CIITA | 0.75 | 0.58 | 0.92 |
| 4 days | CD177 + IL1R2 + CD74 + IFNG + CIITA + CD3D | 0.746 | 0.574 | 0.919 |
| 4 days | CD177 + IL1R2 + CD74 + IFNG + CIITA + CD3D + CTLA4 | 0.75 | 0.575 | 0.925 |
| 4 days | CD177 + IL1R2 + CD74 + IFNG + CIITA + CTLA4 | 0.75 | 0.575 | 0.925 |
| 4 days | CD177 + IL1R2 + CD74 + IFNG + CIITA + TAP2 | 0.762 | 0.595 | 0.928 |
| 4 days | CD177 + IL1R2 + CD74 + IFNG + CIITA + TAP2 + CD3D | 0.746 | 0.574 | 0.918 |
| 4 days | CD177 + IL1R2 + CD74 + IFNG + CIITA + TAP2 + CD3D + | 0.75 | 0.575 | 0.925 |
| | CTLA4 | | | |
| 4 days | CD177 + IL1R2 + CD74 + IFNG + CIITA + TAP2 + CTLA4 | 0.75 | 0.575 | 0.925 |
| 4 days | CD177 + IL1R2 + CD74 + IFNG + CTLA4 | 0.75 | 0.578 | 0.922 |
| 4 days | CD177 + IL1R2 + CD74 + IFNG + CX3CR1 | 0.867 | 0.735 | 0.999 |
| 4 days | CD177 + IL1R2 + CD74 + IFNG + CX3CR1 + CD3D | 0.875 | 0.758 | 0.992 |
| 4 days | CD177 + IL1R2 + CD74 + IFNG + CX3CR1 + CD3D + CTLA4 | 0.902 | 0.802 | 1 |
| 4 days | CD177 + IL1R2 + CD74 + IFNG + CX3CR1 + CIITA | 0.891 | 0.772 | 1 |
| 4 days | CD177 + IL1R2 + CD74 + IFNG + CX3CR1 + CIITA + CD3D | 0.883 | 0.771 | 0.995 |
| 4 days | CD177 + IL1R2 + CD74 + IFNG + CX3CR1 + CIITA + CD3D + | 0.906 | 0.807 | 1 |
| | CTLA4 | | | |
| 4 days | CD177 + IL1R2 + CD74 + IFNG + CX3CR1 + CIITA + CTLA4 | 0.895 | 0.786 | 1 |
| 4 days | CD177 + IL1R2 + CD74 + IFNG + CX3CR1 + CIITA + TAP2 | 0.867 | 0.738 | 0.996 |
| 4 days | CD177 + IL1R2 + CD74 + IFNG + CX3CR1 + CIITA + TAP2 + | 0.883 | 0.771 | 0.995 |
| | CD3D | | | |
| 4 days | CD177 + IL1R2 + CD74 + IFNG + CX3CR1 + CIITA + TAP2 + | 0.895 | 0.789 | 1 |
| | CD3D + CTLA4 | | | |
| 4 days | CD177 + IL1R2 + CD74 + IFNG + CX3CR1 + CIITA + TAP2 + | 0.891 | 0.782 | 1 |
| | CTLA4 | | | |
| 4 days | CD177 + IL1R2 + CD74 + IFNG + CX3CR1 + CTLA4 | 0.891 | 0.778 | 1 |
| 4 days | CD177 + IL1R2 + CD74 + IFNG + CX3CR1 + TAP2 | 0.855 | 0.723 | 0.988 |
| 4 days | CD177 + IL1R2 + CD74 + IFNG + CX3CR1 + TAP2 + CD3D | 0.887 | 0.776 | 0.997 |
| 4 days | CD177 + IL1R2 + CD74 + IFNG + CX3CR1 + TAP2 + CD3D + | 0.895 | 0.789 | 1 |
| | CTLA4 | | | |
| 4 days | CD177 + IL1R2 + CD74 + IFNG + CX3CR1 + TAP2 + CTLA4 | 0.895 | 0.788 | 1 |
| 4 days | CD177 + IL1R2 + CD74 + IFNG + S100A9 | 0.82 | 0.67 | 0.971 |
| 4 days | CD177 + IL1R2 + CD74 + IFNG + S100A9 + CD3D | 0.832 | 0.692 | 0.972 |
| 4 days | CD177 + IL1R2 + CD74 + IFNG + S100A9 + CD3D + CTLA4 | 0.832 | 0.692 | 0.972 |
| 4 days | CD177 + IL1R2 + CD74 + IFNG + S100A9 + CIITA | 0.824 | 0.675 | 0.974 |
| 4 days | CD177 + IL1R2 + CD74 + IFNG + S100A9 + CIITA + CD3D | 0.828 | 0.686 | 0.97 |
| 4 days | CD177 + IL1R2 + CD74 + IFNG + S100A9 + CIITA + CD3D + | 0.828 | 0.687 | 0.97 |
| | CTLA4 | | | |
| 4 days | CD177 + IL1R2 + CD74 + IFNG + S100A9 + CIITA + CTLA4 | 0.809 | 0.654 | 0.963 |
| 4 days | CD177 + IL1R2 + CD74 + IFNG + S100A9 + CIITA + TAP2 | 0.828 | 0.678 | 0.979 |
| 4 days | CD177 + IL1R2 + CD74 + IFNG + S100A9 + CIITA + TAP2 + | 0.855 | 0.723 | 0.988 |
| | CD3D | | | |
| 4 days | CD177 + IL1R2 + CD74 + IFNG + S100A9 + CIITA + TAP2 + | 0.852 | 0.717 | 0.986 |
| | CD3D + CTLA4 | | | |
| 4 days | CD177 + IL1R2 + CD74 + IFNG + S100A9 + CIITA + TAP2 + | 0.82 | 0.669 | 0.972 |
| | CTLA4 | | | |
| 4 days | CD177 + IL1R2 + CD74 + IFNG + S100A9 + CTLA4 | 0.816 | 0.667 | 0.966 |
| 4 days | CD177 + IL1R2 + CD74 + IFNG + S100A9 + CX3CR1 | 0.941 | 0.861 | 1 |
| 4 days | CD177 + IL1R2 + CD74 + IFNG + S100A9 + CX3CR1 + CD3D | 0.973 | 0.917 | 1 |
| 4 days | CD177 + IL1R2 + CD74 + IFNG + S100A9 + CX3CR1 + CD3D + | 0.98 | 0.939 | 1 |
| | CTLA4 | | | |
| 4 days | CD177 + IL1R2 + CD74 + IFNG + S100A9 + CX3CR1 + CIITA | 0.938 | 0.851 | 1 |
| 4 days | CD177 + IL1R2 + CD74 + IFNG + S100A9 + CX3CR1 + CIITA + | 0.984 | 0.951 | 1 |
| | CD3D | | | |
| 4 days | CD177 + IL1R2 + CD74 + IFNG + S100A9 + CX3CR1 + CIITA + | 0.969 | 0.921 | 1 |
| | CTLA4 | | | |
| 4 days | CD177 + IL1R2 + CD74 + IFNG + S100A9 + CX3CR1 + CIITA + | 0.938 | 0.851 | 1 |
| | TAP2 | | | |
| 4 days | CD177 + IL1R2 + CD74 + IFNG + S100A9 + CX3CR1 + CIITA + | 0.965 | 0.914 | 1 |
| | TAP2 + CTLA4 | | | |
| 4 days | CD177 + IL1R2 + CD74 + IFNG + S100A9 + CX3CR1 + CTLA4 | 0.969 | 0.921 | 1 |
| 4 days | CD177 + IL1R2 + CD74 + IFNG + S100A9 + CX3CR1 + TAP2 | 0.941 | 0.861 | 1 |
| 4 days | CD177 + IL1R2 + CD74 + IFNG + S100A9 + CX3CR1 + TAP2 + | 0.965 | 0.912 | 1 |
| | CTLA4 | | | |
| 4 days | CD177 + IL1R2 + CD74 + IFNG + S100A9 + TAP2 | 0.832 | 0.685 | 0.979 |
| 4 days | CD177 + IL1R2 + CD74 + IFNG + S100A9 + TAP2 + CD3D | 0.848 | 0.713 | 0.982 |
| 4 days | CD177 + IL1R2 + CD74 + IFNG + S100A9 + TAP2 + CD3D + | 0.848 | 0.713 | 0.982 |
| | CTLA4 | | | |
| 4 days | CD177 + IL1R2 + CD74 + IFNG + S100A9 + TAP2 + CTLA4 | 0.824 | 0.677 | 0.972 |
| 4 days | CD177 + IL1R2 + CD74 + IFNG + TAP2 | 0.754 | 0.585 | 0.922 |
| 4 days | CD177 + IL1R2 + CD74 + IFNG + TAP2 + CD3D | 0.758 | 0.591 | 0.925 |
| 4 days | CD177 + IL1R2 + CD74 + IFNG + TAP2 + CD3D + CTLA4 | 0.754 | 0.583 | 0.925 |
| 4 days | CD177 + IL1R2 + CD74 + IFNG + TAP2 + CTLA4 | 0.746 | 0.573 | 0.92 |
| 4 days | CD177 + IL1R2 + CD74 + S100A9 | 0.828 | 0.674 | 0.983 |
| 4 days | CD177 + IL1R2 + CD74 + S100A9 + CD3D | 0.824 | 0.679 | 0.969 |
| 4 days | CD177 + IL1R2 + CD74 + S100A9 + CD3D + CTLA4 | 0.824 | 0.679 | 0.969 |
| 4 days | CD177 + IL1R2 + CD74 + S100A9 + CIITA | 0.828 | 0.674 | 0.983 |
| 4 days | CD177 + IL1R2 + CD74 + S100A9 + CIITA + CD3D | 0.824 | 0.678 | 0.971 |
| 4 days | CD177 + IL1R2 + CD74 + S100A9 + CIITA + CD3D + CTLA4 | 0.816 | 0.666 | 0.966 |
| 4 days | CD177 + IL1R2 + CD74 + S100A9 + CIITA + CTLA4 | 0.82 | 0.665 | 0.976 |
| 4 days | CD177 + IL1R2 + CD74 + S100A9 + CIITA + TAP2 | 0.828 | 0.672 | 0.984 |
| 4 days | CD177 + IL1R2 + CD74 + S100A9 + CIITA + TAP2 + CD3D | 0.836 | 0.692 | 0.979 |
| 4 days | CD177 + IL1R2 + CD74 + S100A9 + CIITA + TAP2 + CD3D + | 0.84 | 0.698 | 0.982 |
| | CTLA4 | | | |
| 4 days | CD177 + IL1R2 + CD74 + S100A9 + CIITA + TAP2 + CTLA4 | 0.828 | 0.674 | 0.983 |
| 4 days | CD177 + IL1R2 + CD74 + S100A9 + CTLA4 | 0.828 | 0.674 | 0.983 |
| 4 days | CD177 + IL1R2 + CD74 + S100A9 + CX3CR1 | 0.859 | 0.713 | 1 |
| 4 days | CD177 + IL1R2 + CD74 + S100A9 + CX3CR1 + CD3D | 0.891 | 0.766 | 1 |
| 4 days | CD177 + IL1R2 + CD74 + S100A9 + CX3CR1 + CD3D + CTLA4 | 0.895 | 0.775 | 1 |
| 4 days | CD177 + IL1R2 + CD74 + S100A9 + CX3CR1 + CIITA | 0.863 | 0.723 | 1 |
| 4 days | CD177 + IL1R2 + CD74 + S100A9 + CX3CR1 + CIITA + CD3D | 0.902 | 0.788 | 1 |
| 4 days | CD177 + IL1R2 + CD74 + S100A9 + CX3CR1 + CIITA + CD3D + | 0.895 | 0.782 | 1 |
| | CTLA4 | | | |
| 4 days | CD177 + IL1R2 + CD74 + S100A9 + CX3CR1 + CIITA + CTLA4 | 0.871 | 0.736 | 1 |
| 4 days | CD177 + IL1R2 + CD74 + S100A9 + CX3CR1 + CIITA + TAP2 | 0.887 | 0.759 | 1 |
| 4 days | CD177 + IL1R2 + CD74 + S100A9 + CX3CR1 + CIITA + TAP2 + | 0.906 | 0.799 | 1 |
| | CD3D | | | |
| 4 days | CD177 + IL1R2 + CD74 + S100A9 + CX3CR1 + CIITA + TAP2 + | 0.906 | 0.804 | 1 |
| | CD3D + CTLA4 | | | |
| 4 days | CD177 + IL1R2 + CD74 + S100A9 + CX3CR1 + CIITA + TAP2 + | 0.887 | 0.759 | 1 |
| | CTLA4 | | | |
| 4 days | CD177 + IL1R2 + CD74 + S100A9 + CX3CR1 + CTLA4 | 0.863 | 0.722 | 1 |
| 4 days | CD177 + IL1R2 + CD74 + S100A9 + CX3CR1 + TAP2 | 0.867 | 0.726 | 1 |
| 4 days | CD177 + IL1R2 + CD74 + S100A9 + CX3CR1 + TAP2 + CD3D | 0.895 | 0.774 | 1 |
| 4 days | CD177 + IL1R2 + CD74 + S100A9 + CX3CR1 + TAP2 + CD3D + | 0.887 | 0.765 | 1 |
| | CTLA4 | | | |
| 4 days | CD177 + IL1R2 + CD74 + S100A9 + CX3CR1 + TAP2 + CTLA4 | 0.875 | 0.737 | 1 |
| 4 days | CD177 + IL1R2 + CD74 + S100A9 + TAP2 | 0.828 | 0.672 | 0.984 |
| 4 days | CD177 + IL1R2 + CD74 + S100A9 + TAP2 + CD3D | 0.836 | 0.692 | 0.979 |
| 4 days | CD177 + IL1R2 + CD74 + S100A9 + TAP2 + CD3D + CTLA4 | 0.832 | 0.688 | 0.976 |
| 4 days | CD177 + IL1R2 + CD74 + S100A9 + TAP2 + CTLA4 | 0.828 | 0.674 | 0.983 |
| 4 days | CD177 + IL1R2 + CD74 + TAP2 | 0.734 | 0.554 | 0.915 |
| 4 days | CD177 + IL1R2 + CD74 + TAP2 + CD3D | 0.742 | 0.568 | 0.917 |
| 4 days | CD177 + IL1R2 + CD74 + TAP2 + CD3D + CTLA4 | 0.75 | 0.576 | 0.924 |
| 4 days | CD177 + IL1R2 + CD74 + TAP2 + CTLA4 | 0.746 | 0.569 | 0.923 |
| 4 days | CD177 + IL1R2 + CIITA | 0.707 | 0.521 | 0.893 |
| 4 days | CD177 + IL1R2 + CIITA + CD3D | 0.711 | 0.524 | 0.898 |
| 4 days | CD177 + IL1R2 + CIITA + CD3D + CTLA4 | 0.695 | 0.508 | 0.883 |
| 4 days | CD177 + IL1R2 + CIITA + CTLA4 | 0.699 | 0.512 | 0.887 |
| 4 days | CD177 + IL1R2 + CIITA + TAP2 | 0.707 | 0.515 | 0.899 |
| 4 days | CD177 + IL1R2 + CIITA + TAP2 + CD3D | 0.727 | 0.543 | 0.91 |
| 4 days | CD177 + IL1R2 + CIITA + TAP2 + CD3D + CTLA4 | 0.719 | 0.534 | 0.903 |
| 4 days | CD177 + IL1R2 + CIITA + TAP2 + CTLA4 | 0.727 | 0.544 | 0.91 |
| 4 days | CD177 + IL1R2 + CTLA4 | 0.688 | 0.493 | 0.882 |
| 4 days | CD177 + IL1R2 + CX3CR1 | 0.695 | 0.507 | 0.884 |
| 4 days | CD177 + IL1R2 + CX3CR1 + CD3D | 0.703 | 0.518 | 0.889 |
| 4 days | CD177 + IL1R2 + CX3CR1 + CD3D + CTLA4 | 0.691 | 0.501 | 0.882 |
| 4 days | CD177 + IL1R2 + CX3CR1 + CIITA | 0.742 | 0.562 | 0.923 |
| 4 days | CD177 + IL1R2 + CX3CR1 + CIITA + CD3D | 0.715 | 0.519 | 0.911 |
| 4 days | CD177 + IL1R2 + CX3CR1 + CIITA + CD3D + CTLA4 | 0.711 | 0.515 | 0.907 |
| 4 days | CD177 + IL1R2 + CX3CR1 + CIITA + CTLA4 | 0.711 | 0.52 | 0.902 |
| 4 days | CD177 + IL1R2 + CX3CR1 + CIITA + TAP2 | 0.777 | 0.616 | 0.939 |
| 4 days | CD177 + IL1R2 + CX3CR1 + CIITA + TAP2 + CD3D | 0.789 | 0.629 | 0.949 |
| 4 days | CD177 + IL1R2 + CX3CR1 + CIITA + TAP2 + CD3D + CTLA4 | 0.777 | 0.612 | 0.942 |
| 4 days | CD177 + IL1R2 + CX3CR1 + CIITA + TAP2 + CTLA4 | 0.781 | 0.62 | 0.943 |
| 4 days | CD177 + IL1R2 + CX3CR1 + CTLA4 | 0.711 | 0.523 | 0.899 |
| 4 days | CD177 + IL1R2 + CX3CR1 + TAP2 | 0.734 | 0.557 | 0.912 |
| 4 days | CD177 + IL1R2 + CX3CR1 + TAP2 + CD3D | 0.746 | 0.575 | 0.917 |
| 4 days | CD177 + IL1R2 + CX3CR1 + TAP2 + CD3D + CTLA4 | 0.75 | 0.58 | 0.92 |
| 4 days | CD177 + IL1R2 + CX3CR1 + TAP2 + CTLA4 | 0.742 | 0.57 | 0.915 |
| 4 days | CD177 + IL1R2 + IFNG | 0.734 | 0.557 | 0.912 |
| 4 days | CD177 + IL1R2 + IFNG + CD3D | 0.738 | 0.56 | 0.917 |
| 4 days | CD177 + IL1R2 + IFNG + CD3D + CTLA4 | 0.738 | 0.56 | 0.917 |
| 4 days | CD177 + IL1R2 + IFNG + CIITA | 0.734 | 0.559 | 0.909 |
| 4 days | CD177 + IL1R2 + IFNG + CIITA + CD3D | 0.742 | 0.567 | 0.917 |
| 4 days | CD177 + IL1R2 + IFNG + CIITA + CD3D + CTLA4 | 0.738 | 0.56 | 0.917 |
| 4 days | CD177 + IL1R2 + IFNG + CIITA + CTLA4 | 0.734 | 0.555 | 0.913 |
| 4 days | CD177 + IL1R2 + IFNG + CIITA + TAP2 | 0.738 | 0.561 | 0.916 |
| 4 days | CD177 + IL1R2 + IFNG + CIITA + TAP2 + CD3D | 0.746 | 0.57 | 0.923 |
| 4 days | CD177 + IL1R2 + IFNG + CIITA + TAP2 + CD3D + CTLA4 | 0.754 | 0.578 | 0.93 |
| 4 days | CD177 + IL1R2 + IFNG + CIITA + TAP2 + CTLA4 | 0.75 | 0.573 | 0.927 |
| 4 days | CD177 + IL1R2 + IFNG + CTLA4 | 0.738 | 0.56 | 0.917 |
| 4 days | CD177 + IL1R2 + IFNG + CX3CR1 | 0.844 | 0.699 | 0.988 |
| 4 days | CD177 + IL1R2 + IFNG + CX3CR1 + CD3D | 0.844 | 0.705 | 0.983 |
| 4 days | CD177 + IL1R2 + IFNG + CX3CR1 + CD3D + CTLA4 | 0.871 | 0.749 | 0.994 |
| 4 days | CD177 + IL1R2 + IFNG + CX3CR1 + CIITA | 0.863 | 0.725 | 1 |
| 4 days | CD177 + IL1R2 + IFNG + CX3CR1 + CIITA + CD3D | 0.859 | 0.73 | 0.989 |
| 4 days | CD177 + IL1R2 + IFNG + CX3CR1 + CIITA + CD3D + CTLA4 | 0.879 | 0.758 | 1 |
| 4 days | CD177 + IL1R2 + IFNG + CX3CR1 + CIITA + CTLA4 | 0.875 | 0.75 | 1 |
| 4 days | CD177 + IL1R2 + IFNG + CX3CR1 + CIITA + TAP2 | 0.867 | 0.735 | 1 |
| 4 days | CD177 + IL1R2 + IFNG + CX3CR1 + CIITA + TAP2 + CD3D | 0.863 | 0.734 | 0.992 |
| 4 days | CD177 + IL1R2 + IFNG + CX3CR1 + CIITA + TAP2 + CD3D + | 0.871 | 0.743 | 1 |
| | CTLA4 | | | |
| 4 days | CD177 + IL1R2 + IFNG + CX3CR1 + CIITA + TAP2 + CTLA4 | 0.875 | 0.749 | 1 |
| 4 days | CD177 + IL1R2 + IFNG + CX3CR1 + CTLA4 | 0.871 | 0.749 | 0.994 |
| 4 days | CD177 + IL1R2 + IFNG + CX3CR1 + TAP2 | 0.836 | 0.697 | 0.975 |
| 4 days | CD177 + IL1R2 + IFNG + CX3CR1 + TAP2 + CD3D | 0.852 | 0.72 | 0.983 |
| 4 days | CD177 + IL1R2 + IFNG + CX3CR1 + TAP2 + CD3D + CTLA4 | 0.879 | 0.756 | 1 |
| 4 days | CD177 + IL1R2 + IFNG + CX3CR1 + TAP2 + CTLA4 | 0.879 | 0.756 | 1 |
| 4 days | CD177 + IL1R2 + IFNG + S100A9 | 0.828 | 0.68 | 0.976 |
| 4 days | CD177 + IL1R2 + IFNG + S100A9 + CD3D | 0.832 | 0.693 | 0.971 |
| 4 days | CD177 + IL1R2 + IFNG + S100A9 + CD3D + CTLA4 | 0.828 | 0.687 | 0.969 |
| 4 days | CD177 + IL1R2 + IFNG + S100A9 + CIITA | 0.816 | 0.663 | 0.97 |
| 4 days | CD177 + IL1R2 + IFNG + S100A9 + CIITA + CD3D | 0.828 | 0.686 | 0.97 |
| 4 days | CD177 + IL1R2 + IFNG + S100A9 + CIITA + CD3D + CTLA4 | 0.824 | 0.682 | 0.967 |
| 4 days | CD177 + IL1R2 + IFNG + S100A9 + CIITA + CTLA4 | 0.809 | 0.655 | 0.962 |
| 4 days | CD177 + IL1R2 + IFNG + S100A9 + CIITA + TAP2 | 0.828 | 0.678 | 0.979 |
| 4 days | CD177 + IL1R2 + IFNG + S100A9 + CIITA + TAP2 + CD3D | 0.855 | 0.723 | 0.988 |
| 4 days | CD177 + IL1R2 + IFNG + S100A9 + CIITA + TAP2 + CD3D + | 0.855 | 0.723 | 0.988 |
| | CTLA4 | | | |
| 4 days | CD177 + IL1R2 + IFNG + S100A9 + CIITA + TAP2 + CTLA4 | 0.82 | 0.669 | 0.972 |
| 4 days | CD177 + IL1R2 + IFNG + S100A9 + CTLA4 | 0.816 | 0.669 | 0.964 |
| 4 days | CD177 + IL1R2 + IFNG + S100A9 + CX3CR1 | 0.938 | 0.851 | 1 |
| 4 days | CD177 + IL1R2 + IFNG + S100A9 + CX3CR1 + CD3D | 0.973 | 0.917 | 1 |
| 4 days | CD177 + IL1R2 + IFNG + S100A9 + CX3CR1 + CD3D + CTLA4 | 0.977 | 0.928 | 1 |
| 4 days | CD177 + IL1R2 + IFNG + S100A9 + CX3CR1 + CIITA | 0.938 | 0.851 | 1 |
| 4 days | CD177 + IL1R2 + IFNG + S100A9 + CX3CR1 + CIITA + CD3D | 0.984 | 0.951 | 1 |
| 4 days | CD177 + IL1R2 + IFNG + S100A9 + CX3CR1 + CIITA + CD3D + | 0.98 | 0.939 | 1 |
| | CTLA4 | | | |
| 4 days | CD177 + IL1R2 + IFNG + S100A9 + CX3CR1 + CIITA + CTLA4 | 0.965 | 0.914 | 1 |
| 4 days | CD177 + IL1R2 + IFNG + S100A9 + CX3CR1 + CIITA + TAP2 | 0.938 | 0.851 | 1 |
| 4 days | CD177 + IL1R2 + IFNG + S100A9 + CX3CR1 + CIITA + TAP2 + | 0.965 | 0.914 | 1 |
| | CTLA4 | | | |
| 4 days | CD177 + IL1R2 + IFNG + S100A9 + CX3CR1 + CTLA4 | 0.969 | 0.921 | 1 |
| 4 days | CD177 + IL1R2 + IFNG + S100A9 + CX3CR1 + TAP2 | 0.938 | 0.851 | 1 |
| 4 days | CD177 + IL1R2 + IFNG + S100A9 + CX3CR1 + TAP2 + CD3D | 0.984 | 0.951 | 1 |
| 4 days | CD177 + IL1R2 + IFNG + S100A9 + CX3CR1 + TAP2 + CTLA4 | 0.965 | 0.912 | 1 |
| 4 days | CD177 + IL1R2 + IFNG + S100A9 + TAP2 | 0.836 | 0.69 | 0.982 |
| 4 days | CD177 + IL1R2 + IFNG + S100A9 + TAP2 + CD3D | 0.84 | 0.702 | 0.978 |
| 4 days | CD177 + IL1R2 + IFNG + S100A9 + TAP2 + CD3D + CTLA4 | 0.844 | 0.707 | 0.98 |
| 4 days | CD177 + IL1R2 + IFNG + S100A9 + TAP2 + CTLA4 | 0.828 | 0.681 | 0.975 |
| 4 days | CD177 + IL1R2 + IFNG + TAP2 | 0.758 | 0.585 | 0.931 |
| 4 days | CD177 + IL1R2 + IFNG + TAP2 + CD3D | 0.734 | 0.552 | 0.917 |
| 4 days | CD177 + IL1R2 + IFNG + TAP2 + CD3D + CTLA4 | 0.754 | 0.578 | 0.93 |
| 4 days | CD177 + IL1R2 + IFNG + TAP2 + CTLA4 | 0.758 | 0.582 | 0.933 |
| 4 days | CD177 + IL1R2 + S100A9 | 0.824 | 0.665 | 0.983 |
| 4 days | CD177 + IL1R2 + S100A9 + CD3D | 0.816 | 0.666 | 0.967 |
| 4 days | CD177 + IL1R2 + S100A9 + CD3D + CTLA4 | 0.816 | 0.666 | 0.967 |
| 4 days | CD177 + IL1R2 + S100A9 + CIITA | 0.82 | 0.66 | 0.98 |
| 4 days | CD177 + IL1R2 + S100A9 + CIITA + CD3D | 0.832 | 0.686 | 0.978 |
| 4 days | CD177 + IL1R2 + S100A9 + CIITA + CD3D + CTLA4 | 0.836 | 0.693 | 0.979 |
| 4 days | CD177 + IL1R2 + S100A9 + CIITA + CTLA4 | 0.82 | 0.665 | 0.976 |
| 4 days | CD177 + IL1R2 + S100A9 + CIITA + TAP2 | 0.82 | 0.659 | 0.981 |
| 4 days | CD177 + IL1R2 + S100A9 + CIITA + TAP2 + CD3D | 0.836 | 0.692 | 0.979 |
| 4 days | CD177 + IL1R2 + S100A9 + CIITA + TAP2 + CD3D + CTLA4 | 0.84 | 0.698 | 0.982 |
| 4 days | CD177 + IL1R2 + S100A9 + CIITA + TAP2 + CTLA4 | 0.824 | 0.669 | 0.979 |
| 4 days | CD177 + IL1R2 + S100A9 + CTLA4 | 0.82 | 0.665 | 0.976 |
| 4 days | CD177 + IL1R2 + S100A9 + CX3CR1 | 0.859 | 0.716 | 1 |
| 4 days | CD177 + IL1R2 + S100A9 + CX3CR1 + CD3D | 0.891 | 0.766 | 1 |
| 4 days | CD177 + IL1R2 + S100A9 + CX3CR1 + CD3D + CTLA4 | 0.887 | 0.764 | 1 |
| 4 days | CD177 + IL1R2 + S100A9 + CX3CR1 + CIITA | 0.863 | 0.723 | 1 |
| 4 days | CD177 + IL1R2 + S100A9 + CX3CR1 + CIITA + CD3D | 0.898 | 0.781 | 1 |
| 4 days | CD177 + IL1R2 + S100A9 + CX3CR1 + CIITA + CD3D + CTLA4 | 0.895 | 0.779 | 1 |
| 4 days | CD177 + IL1R2 + S100A9 + CX3CR1 + CIITA + CTLA4 | 0.867 | 0.728 | 1 |
| 4 days | CD177 + IL1R2 + S100A9 + CX3CR1 + CIITA + TAP2 | 0.879 | 0.742 | 1 |
| 4 days | CD177 + IL1R2 + S100A9 + CX3CR1 + CIITA + TAP2 + CD3D | 0.906 | 0.796 | 1 |
| 4 days | CD177 + IL1R2 + S100A9 + CX3CR1 + CIITA + TAP2 + CD3D + | 0.898 | 0.79 | 1 |
| | CTLA4 | | | |
| 4 days | CD177 + IL1R2 + S100A9 + CX3CR1 + CIITA + TAP2 + CTLA4 | 0.879 | 0.742 | 1 |
| 4 days | CD177 + IL1R2 + S100A9 + CX3CR1 + CTLA4 | 0.863 | 0.722 | 1 |
| 4 days | CD177 + IL1R2 + S100A9 + CX3CR1 + TAP2 | 0.871 | 0.735 | 1 |
| 4 days | CD177 + IL1R2 + S100A9 + CX3CR1 + TAP2 + CD3D | 0.898 | 0.781 | 1 |
| 4 days | CD177 + IL1R2 + S100A9 + CX3CR1 + TAP2 + CD3D + CTLA4 | 0.891 | 0.775 | 1 |
| 4 days | CD177 + IL1R2 + S100A9 + CX3CR1 + TAP2 + CTLA4 | 0.875 | 0.737 | 1 |
| 4 days | CD177 + IL1R2 + S100A9 + TAP2 | 0.82 | 0.659 | 0.981 |
| 4 days | CD177 + IL1R2 + S100A9 + TAP2 + CD3D | 0.828 | 0.683 | 0.973 |
| 4 days | CD177 + IL1R2 + S100A9 + TAP2 + CD3D + CTLA4 | 0.828 | 0.683 | 0.973 |
| 4 days | CD177 + IL1R2 + S100A9 + TAP2 + CTLA4 | 0.828 | 0.674 | 0.983 |
| 4 days | CD177 + IL1R2 + TAP2 | 0.711 | 0.521 | 0.901 |
| 4 days | CD177 + IL1R2 + TAP2 + CD3D | 0.707 | 0.516 | 0.898 |
| 4 days | CD177 + IL1R2 + TAP2 + CD3D + CTLA4 | 0.727 | 0.547 | 0.906 |
| 4 days | CD177 + IL1R2 + TAP2 + CTLA4 | 0.723 | 0.542 | 0.904 |
| 4 days | CD177 + S100A9 + CD3D | 0.746 | 0.561 | 0.931 |
| 4 days | CD177 + S100A9 + CD3D + CTLA4 | 0.773 | 0.596 | 0.951 |
| 4 days | CD177 + S100A9 + CIITA | 0.742 | 0.554 | 0.931 |
| 4 days | CD177 + S100A9 + CIITA + CD3D | 0.734 | 0.545 | 0.924 |
| 4 days | CD177 + S100A9 + CIITA + CD3D + CTLA4 | 0.754 | 0.573 | 0.935 |
| 4 days | CD177 + S100A9 + CIITA + CTLA4 | 0.746 | 0.562 | 0.93 |
| 4 days | CD177 + S100A9 + CIITA + TAP2 | 0.738 | 0.544 | 0.932 |
| 4 days | CD177 + S100A9 + CIITA + TAP2 + CD3D | 0.73 | 0.534 | 0.927 |
| 4 days | CD177 + S100A9 + CIITA + TAP2 + CD3D + CTLA4 | 0.746 | 0.559 | 0.933 |
| 4 days | CD177 + S100A9 + CIITA + TAP2 + CTLA4 | 0.746 | 0.559 | 0.933 |
| 4 days | CD177 + S100A9 + CTLA4 | 0.742 | 0.557 | 0.928 |
| 4 days | CD177 + S100A9 + CX3CR1 | 0.855 | 0.716 | 0.995 |
| 4 days | CD177 + S100A9 + CX3CR1 + CD3D | 0.855 | 0.716 | 0.995 |
| 4 days | CD177 + S100A9 + CX3CR1 + CD3D + CTLA4 | 0.859 | 0.724 | 0.994 |
| 4 days | CD177 + S100A9 + CX3CR1 + CIITA | 0.852 | 0.706 | 0.997 |
| 4 days | CD177 + S100A9 + CX3CR1 + CIITA + CD3D | 0.859 | 0.721 | 0.998 |
| 4 days | CD177 + S100A9 + CX3CR1 + CIITA + CD3D + CTLA4 | 0.852 | 0.716 | 0.987 |
| 4 days | CD177 + S100A9 + CX3CR1 + CIITA + CTLA4 | 0.859 | 0.717 | 1 |
| 4 days | CD177 + S100A9 + CX3CR1 + CIITA + TAP2 | 0.859 | 0.72 | 0.999 |
| 4 days | CD177 + S100A9 + CX3CR1 + CIITA + TAP2 + CD3D | 0.859 | 0.721 | 0.998 |
| 4 days | CD177 + S100A9 + CX3CR1 + CIITA + TAP2 + CD3D + CTLA4 | 0.855 | 0.721 | 0.99 |
| 4 days | CD177 + S100A9 + CX3CR1 + CIITA + TAP2 + CTLA4 | 0.863 | 0.725 | 1 |
| 4 days | CD177 + S100A9 + CX3CR1 + CTLA4 | 0.855 | 0.713 | 0.998 |
| 4 days | CD177 + S100A9 + CX3CR1 + TAP2 | 0.859 | 0.719 | 1 |
| 4 days | CD177 + S100A9 + CX3CR1 + TAP2 + CD3D | 0.855 | 0.716 | 0.995 |
| 4 days | CD177 + S100A9 + CX3CR1 + TAP2 + CD3D + CTLA4 | 0.859 | 0.724 | 0.994 |
| 4 days | CD177 + S100A9 + CX3CR1 + TAP2 + CTLA4 | 0.867 | 0.728 | 1 |
| 4 days | CD177 + S100A9 + TAP2 | 0.75 | 0.561 | 0.939 |
| 4 days | CD177 + S100A9 + TAP2 + CD3D | 0.75 | 0.563 | 0.937 |
| 4 days | CD177 + S100A9 + TAP2 + CD3D + CTLA4 | 0.766 | 0.586 | 0.945 |
| 4 days | CD177 + S100A9 + TAP2 + CTLA4 | 0.762 | 0.58 | 0.943 |
| 4 days | CD177 + TAP2 + CD3D | 0.691 | 0.495 | 0.888 |
| 4 days | CD177 + TAP2 + CD3D + CTLA4 | 0.719 | 0.528 | 0.909 |
| 7 days | CD177 + IL6 | 0.729 | 0.564 | 0.893 |
| 7 days | CD177 + LILRB2 | 0.729 | 0.565 | 0.892 |
| 7 days | CD177 + MDC1 | 0.729 | 0.564 | 0.894 |
| 7 days | CD177 + CD3D | 0.731 | 0.567 | 0.895 |
| 7 days | CD177 + IL1RN | 0.731 | 0.565 | 0.897 |
| 7 days | CD177 + OAS2 | 0.731 | 0.561 | 0.902 |
| 7 days | CD177 + TAP2 | 0.731 | 0.563 | 0.9 |
| 7 days | CD177 + IL7R | 0.737 | 0.573 | 0.901 |
| 7 days | CD177 + GATA3 | 0.737 | 0.573 | 0.9 |
| 7 days | CD177 + GSN | 0.737 | 0.571 | 0.902 |
| 7 days | CD177 + TBX21 | 0.737 | 0.574 | 0.9 |
| 7 days | CD177 + ALOX5 | 0.74 | 0.574 | 0.905 |
| 7 days | CD177 + S100A9 | 0.742 | 0.579 | 0.905 |
| 7 days | CD177 + FCGR1A | 0.745 | 0.582 | 0.908 |
| 7 days | CD177 + FLT1 | 0.745 | 0.581 | 0.91 |
| 7 days | CD177 + RORC | 0.745 | 0.578 | 0.912 |
| 7 days | CD177 + CCNB1IP1 | 0.748 | 0.588 | 0.908 |
| 7 days | CD177 + TIM3 | 0.753 | 0.595 | 0.912 |
| 7 days | CD177 + ZAP70 | 0.756 | 0.592 | 0.92 |
| 7 days | CD177 + IL18 | 0.762 | 0.603 | 0.92 |
| 7 days | CD177 + TDRD9 | 0.762 | 0.6 | 0.924 |
| 7 days | CD177 + IFNG | 0.765 | 0.608 | 0.921 |
| 7 days | CD177 + GNLY | 0.781 | 0.633 | 0.929 |
| 7 days | CD177 + CIITA | 0.806 | 0.656 | 0.956 |
| 7 days | CD177 + C3AR1 + CD3D + CTLA4 | 0.731 | 0.571 | 0.892 |
| 7 days | CD177 + C3AR1 + CD74 | 0.812 | 0.669 | 0.954 |
| 7 days | CD177 + C3AR1 + CD74 + CD3D | 0.803 | 0.661 | 0.946 |
| 7 days | CD177 + C3AR1 + CD74 + CD3D + CTLA4 | 0.803 | 0.661 | 0.946 |
| 7 days | CD177 + C3AR1 + CD74 + CIITA | 0.87 | 0.752 | 0.987 |
| 7 days | CD177 + C3AR1 + CD74 + CIITA + CD3D | 0.875 | 0.76 | 0.991 |
| 7 days | CD177 + C3AR1 + CD74 + CIITA + CD3D + CTLA4 | 0.889 | 0.787 | 0.992 |
| 7 days | CD177 + C3AR1 + CD74 + CIITA + CTLA4 | 0.875 | 0.765 | 0.985 |
| 7 days | CD177 + C3AR1 + CD74 + CIITA + TAP2 | 0.87 | 0.752 | 0.987 |
| 7 days | CD177 + C3AR1 + CD74 + CIITA + TAP2 + CD3D | 0.87 | 0.753 | 0.987 |
| 7 days | CD177 + C3AR1 + CD74 + CIITA + TAP2 + CD3D + CTLA4 | 0.898 | 0.8 | 0.995 |
| 7 days | CD177 + C3AR1 + CD74 + CIITA + TAP2 + CTLA4 | 0.873 | 0.761 | 0.985 |
| 7 days | CD177 + C3AR1 + CD74 + CTLA4 | 0.817 | 0.678 | 0.956 |
| 7 days | CD177 + C3AR1 + CD74 + CX3CR1 | 0.823 | 0.684 | 0.961 |
| 7 days | CD177 + C3AR1 + CD74 + CX3CR1 + CD3D | 0.812 | 0.671 | 0.952 |
| 7 days | CD177 + C3AR1 + CD74 + CX3CR1 + CD3D + CTLA4 | 0.812 | 0.672 | 0.951 |
| 7 days | CD177 + C3AR1 + CD74 + CX3CR1 + CIITA | 0.878 | 0.767 | 0.99 |
| 7 days | CD177 + C3AR1 + CD74 + CX3CR1 + CIITA + CD3D | 0.878 | 0.767 | 0.989 |
| 7 days | CD177 + C3AR1 + CD74 + CX3CR1 + CIITA + CD3D + CTLA4 | 0.892 | 0.792 | 0.992 |
| 7 days | CD177 + C3AR1 + CD74 + CX3CR1 + CIITA + CTLA4 | 0.875 | 0.763 | 0.988 |
| 7 days | CD177 + C3AR1 + CD74 + CX3CR1 + CIITA + TAP2 | 0.875 | 0.762 | 0.989 |
| 7 days | CD177 + C3AR1 + CD74 + CX3CR1 + CIITA + TAP2 + CD3D | 0.873 | 0.758 | 0.987 |
| 7 days | CD177 + C3AR1 + CD74 + CX3CR1 + CIITA + TAP2 + CD3D + | 0.898 | 0.801 | 0.994 |
| | CTLA4 | | | |
| 7 days | CD177 + C3AR1 + CD74 + CX3CR1 + CIITA + TAP2 + CTLA4 | 0.875 | 0.763 | 0.987 |
| 7 days | CD177 + C3AR1 + CD74 + CX3CR1 + CTLA4 | 0.823 | 0.683 | 0.962 |
| 7 days | CD177 + C3AR1 + CD74 + CX3CR1 + TAP2 | 0.817 | 0.675 | 0.96 |
| 7 days | CD177 + C3AR1 + CD74 + CX3CR1 + TAP2 + CD3D | 0.82 | 0.684 | 0.956 |
| 7 days | CD177 + C3AR1 + CD74 + CX3CR1 + TAP2 + CD3D + CTLA4 | 0.82 | 0.684 | 0.956 |
| 7 days | CD177 + C3AR1 + CD74 + CX3CR1 + TAP2 + CTLA4 | 0.825 | 0.685 | 0.966 |
| 7 days | CD177 + C3AR1 + CD74 + IFNG | 0.842 | 0.719 | 0.965 |
| 7 days | CD177 + C3AR1 + CD74 + IFNG + CD3D | 0.839 | 0.715 | 0.964 |
| 7 days | CD177 + C3AR1 + CD74 + IFNG + CD3D + CTLA4 | 0.845 | 0.723 | 0.967 |
| 7 days | CD177 + C3AR1 + CD74 + IFNG + CIITA | 0.892 | 0.791 | 0.993 |
| 7 days | CD177 + C3AR1 + CD74 + IFNG + CIITA + CD3D | 0.892 | 0.791 | 0.993 |
| 7 days | CD177 + C3AR1 + CD74 + IFNG + CIITA + CD3D + CTLA4 | 0.892 | 0.795 | 0.989 |
| 7 days | CD177 + C3AR1 + CD74 + IFNG + CIITA + CTLA4 | 0.886 | 0.786 | 0.987 |
| 7 days | CD177 + C3AR1 + CD74 + IFNG + CIITA + TAP2 | 0.892 | 0.792 | 0.992 |
| 7 days | CD177 + C3AR1 + CD74 + IFNG + CIITA + TAP2 + CD3D | 0.892 | 0.792 | 0.992 |
| 7 days | CD177 + C3AR1 + CD74 + IFNG + CIITA + TAP2 + CD3D + | 0.892 | 0.794 | 0.99 |
| | CTLA4 | | | |
| 7 days | CD177 + C3AR1 + CD74 + IFNG + CIITA + TAP2 + CTLA4 | 0.9 | 0.807 | 0.994 |
| 7 days | CD177 + C3AR1 + CD74 + IFNG + CTLA4 | 0.839 | 0.715 | 0.963 |
| 7 days | CD177 + C3AR1 + CD74 + IFNG + CX3CR1 | 0.881 | 0.775 | 0.986 |
| 7 days | CD177 + C3AR1 + CD74 + IFNG + CX3CR1 + CD3D | 0.886 | 0.784 | 0.989 |
| 7 days | CD177 + C3AR1 + CD74 + IFNG + CX3CR1 + CD3D + CTLA4 | 0.886 | 0.782 | 0.991 |
| 7 days | CD177 + C3AR1 + CD74 + IFNG + CX3CR1 + CIITA | 0.917 | 0.827 | 1 |
| 7 days | CD177 + C3AR1 + CD74 + IFNG + CX3CR1 + CIITA + CD3D | 0.906 | 0.808 | 1 |
| 7 days | CD177 + C3AR1 + CD74 + IFNG + CX3CR1 + CIITA + CD3D + | 0.911 | 0.817 | 1 |
| | CTLA4 | | | |
| 7 days | CD177 + C3AR1 + CD74 + IFNG + CX3CR1 + CIITA + CTLA4 | 0.917 | 0.827 | 1 |
| 7 days | CD177 + C3AR1 + CD74 + IFNG + CX3CR1 + CIITA + TAP2 | 0.917 | 0.827 | 1 |
| 7 days | CD177 + C3AR1 + CD74 + IFNG + CX3CR1 + CIITA + TAP2 + | 0.914 | 0.822 | 1 |
| | CD3D | | | |
| 7 days | CD177 + C3AR1 + CD74 + IFNG + CX3CR1 + CIITA + TAP2 + | 0.928 | 0.844 | 1 |
| | CD3D + CTLA4 | | | |
| 7 days | CD177 + C3AR1 + CD74 + IFNG + CX3CR1 + CIITA + TAP2 + | 0.914 | 0.823 | 1 |
| | CTLA4 | | | |
| 7 days | CD177 + C3AR1 + CD74 + IFNG + CX3CR1 + CTLA4 | 0.881 | 0.774 | 0.988 |
| 7 days | CD177 + C3AR1 + CD74 + IFNG + CX3CR1 + TAP2 | 0.881 | 0.775 | 0.986 |
| 7 days | CD177 + C3AR1 + CD74 + IFNG + CX3CR1 + TAP2 + CD3D | 0.892 | 0.79 | 0.994 |
| 7 days | CD177 + C3AR1 + CD74 + IFNG + CX3CR1 + TAP2 + CD3D + | 0.889 | 0.786 | 0.992 |
| | CTLA4 | | | |
| 7 days | CD177 + C3AR1 + CD74 + IFNG + CX3CR1 + TAP2 + CTLA4 | 0.878 | 0.768 | 0.988 |
| 7 days | CD177 + C3AR1 + CD74 + IFNG + S100A9 | 0.842 | 0.72 | 0.965 |
| 7 days | CD177 + C3AR1 + CD74 + IFNG + S100A9 + CD3D | 0.845 | 0.723 | 0.967 |
| 7 days | CD177 + C3AR1 + CD74 + IFNG + S100A9 + CD3D + CTLA4 | 0.842 | 0.719 | 0.965 |
| 7 days | CD177 + C3AR1 + CD74 + IFNG + S100A9 + CIITA | 0.889 | 0.789 | 0.989 |
| 7 days | CD177 + C3AR1 + CD74 + IFNG + S100A9 + CIITA + CD3D | 0.884 | 0.781 | 0.986 |
| 7 days | CD177 + C3AR1 + CD74 + IFNG + S100A9 + CIITA + CD3D + | 0.886 | 0.786 | 0.987 |
| | CTLA4 | | | |
| 7 days | CD177 + C3AR1 + CD74 + IFNG + S100A9 + CIITA + CTLA4 | 0.889 | 0.79 | 0.988 |
| 7 days | CD177 + C3AR1 + CD74 + IFNG + S100A9 + CIITA + TAP2 | 0.906 | 0.814 | 0.997 |
| 7 days | CD177 + C3AR1 + CD74 + IFNG + S100A9 + CIITA + TAP2 + | 0.906 | 0.814 | 0.997 |
| | CD3D | | | |
| 7 days | CD177 + C3AR1 + CD74 + IFNG + S100A9 + CIITA + TAP2 + | 0.892 | 0.794 | 0.99 |
| | CD3D + CTLA4 | | | |
| 7 days | CD177 + C3AR1 + CD74 + IFNG + S100A9 + CIITA + TAP2 + | 0.895 | 0.798 | 0.992 |
| | CTLA4 | | | |
| 7 days | CD177 + C3AR1 + CD74 + IFNG + S100A9 + CTLA4 | 0.842 | 0.72 | 0.965 |
| 7 days | CD177 + C3AR1 + CD74 + IFNG + S100A9 + CX3CR1 | 0.881 | 0.777 | 0.985 |
| 7 days | CD177 + C3AR1 + CD74 + IFNG + S100A9 + CX3CR1 + CD3D | 0.881 | 0.776 | 0.986 |
| 7 days | CD177 + C3AR1 + CD74 + IFNG + S100A9 + CX3CR1 + CD3D + | 0.886 | 0.784 | 0.989 |
| | CTLA4 | | | |
| 7 days | CD177 + C3AR1 + CD74 + IFNG + S100A9 + CX3CR1 + CIITA | 0.92 | 0.831 | 1 |
| 7 days | CD177 + C3AR1 + CD74 + IFNG + S100A9 + CX3CR1 + CIITA + | 0.914 | 0.821 | 1 |
| | CD3D | | | |
| 7 days | CD177 + C3AR1 + CD74 + IFNG + S100A9 + CX3CR1 + CIITA + | 0.909 | 0.813 | 1 |
| | CD3D + CTLA4 | | | |
| 7 days | CD177 + C3AR1 + CD74 + IFNG + S100A9 + CX3CR1 + CIITA + | 0.922 | 0.835 | 1 |
| | CTLA4 | | | |
| 7 days | CD177 + C3AR1 + CD74 + IFNG + S100A9 + CX3CR1 + CIITA + | 0.922 | 0.834 | 1 |
| | TAP2 | | | |
| 7 days | CD177 + C3AR1 + CD74 + IFNG + S100A9 + CX3CR1 + CIITA + | 0.922 | 0.834 | 1 |
| | TAP2 + CD3D | | | |
| 7 days | CD177 + C3AR1 + CD74 + IFNG + S100A9 + CX3CR1 + CIITA + | 0.925 | 0.84 | 1 |
| | TAP2 + CD3D + CTLA4 | | | |
| 7 days | CD177 + C3AR1 + CD74 + IFNG + S100A9 + CX3CR1 + CIITA + | 0.922 | 0.835 | 1 |
| | TAP2 + CTLA4 | | | |
| 7 days | CD177 + C3AR1 + CD74 + IFNG + S100A9 + CX3CR1 + CTLA4 | 0.884 | 0.778 | 0.989 |
| 7 days | CD177 + C3AR1 + CD74 + IFNG + S100A9 + CX3CR1 + TAP2 | 0.881 | 0.777 | 0.985 |
| 7 days | CD177 + C3AR1 + CD74 + IFNG + S100A9 + CX3CR1 + TAP2 + | 0.878 | 0.771 | 0.985 |
| | CD3D | | | |
| 7 days | CD177 + C3AR1 + CD74 + IFNG + S100A9 + CX3CR1 + TAP2 + | 0.881 | 0.775 | 0.987 |
| | CD3D + CTLA4 | | | |
| 7 days | CD177 + C3AR1 + CD74 + IFNG + S100A9 + CX3CR1 + TAP2 + | 0.878 | 0.769 | 0.987 |
| | CTLA4 | | | |
| 7 days | CD177 + C3AR1 + CD74 + IFNG + S100A9 + TAP2 | 0.842 | 0.718 | 0.967 |
| 7 days | CD177 + C3AR1 + CD74 + IFNG + S100A9 + TAP2 + CD3D | 0.834 | 0.707 | 0.96 |
| 7 days | CD177 + C3AR1 + CD74 + IFNG + S100A9 + TAP2 + CD3D + | 0.839 | 0.715 | 0.964 |
| | CTLA4 | | | |
| 7 days | CD177 + C3AR1 + CD74 + IFNG + S100A9 + TAP2 + CTLA4 | 0.839 | 0.714 | 0.964 |
| 7 days | CD177 + C3AR1 + CD74 + IFNG + TAP2 | 0.845 | 0.722 | 0.968 |
| 7 days | CD177 + C3AR1 + CD74 + IFNG + TAP2 + CD3D | 0.839 | 0.715 | 0.964 |
| 7 days | CD177 + C3AR1 + CD74 + IFNG + TAP2 + CD3D + CTLA4 | 0.839 | 0.715 | 0.964 |
| 7 days | CD177 + C3AR1 + CD74 + IFNG + TAP2 + CTLA4 | 0.839 | 0.714 | 0.964 |
| 7 days | CD177 + C3AR1 + CD74 + S100A9 | 0.825 | 0.685 | 0.966 |
| 7 days | CD177 + C3AR1 + CD74 + S100A9 + CD3D | 0.831 | 0.696 | 0.966 |
| 7 days | CD177 + C3AR1 + CD74 + S100A9 + CD3D + CTLA4 | 0.828 | 0.692 | 0.964 |
| 7 days | CD177 + C3AR1 + CD74 + S100A9 + CIITA | 0.873 | 0.757 | 0.988 |
| 7 days | CD177 + C3AR1 + CD74 + S100A9 + CIITA + CD3D | 0.87 | 0.753 | 0.987 |
| 7 days | CD177 + C3AR1 + CD74 + S100A9 + CIITA + CD3D + CTLA4 | 0.889 | 0.787 | 0.992 |
| 7 days | CD177 + C3AR1 + CD74 + S100A9 + CIITA + CTLA4 | 0.878 | 0.77 | 0.987 |
| 7 days | CD177 + C3AR1 + CD74 + S100A9 + CIITA + TAP2 | 0.878 | 0.765 | 0.991 |
| 7 days | CD177 + C3AR1 + CD74 + S100A9 + CIITA + TAP2 + CD3D | 0.875 | 0.761 | 0.99 |
| 7 days | CD177 + C3AR1 + CD74 + S100A9 + CIITA + TAP2 + CD3D + | 0.9 | 0.802 | 0.998 |
| | CTLA4 | | | |
| 7 days | CD177 + C3AR1 + CD74 + S100A9 + CIITA + TAP2 + CTLA4 | 0.878 | 0.77 | 0.987 |
| 7 days | CD177 + C3AR1 + CD74 + S100A9 + CTLA4 | 0.825 | 0.685 | 0.966 |
| 7 days | CD177 + C3AR1 + CD74 + S100A9 + CX3CR1 | 0.853 | 0.718 | 0.989 |
| 7 days | CD177 + C3AR1 + CD74 + S100A9 + CX3CR1 + CD3D | 0.845 | 0.708 | 0.981 |
| 7 days | CD177 + C3AR1 + CD74 + S100A9 + CX3CR1 + CD3D + CTLA4 | 0.85 | 0.715 | 0.986 |
| 7 days | CD177 + C3AR1 + CD74 + S100A9 + CX3CR1 + CIITA | 0.881 | 0.766 | 0.995 |
| 7 days | CD177 + C3AR1 + CD74 + S100A9 + CX3CR1 + CIITA + CD3D | 0.875 | 0.758 | 0.993 |
| 7 days | CD177 + C3AR1 + CD74 + S100A9 + CX3CR1 + CIITA + CD3D + | 0.889 | 0.784 | 0.994 |
| | CTLA4 | | | |
| 7 days | CD177 + C3AR1 + CD74 + S100A9 + CX3CR1 + CIITA + CTLA4 | 0.875 | 0.763 | 0.988 |
| 7 days | CD177 + C3AR1 + CD74 + S100A9 + CX3CR1 + CIITA + TAP2 | 0.881 | 0.769 | 0.993 |
| 7 days | CD177 + C3AR1 + CD74 + S100A9 + CX3CR1 + CIITA + TAP2 + | 0.878 | 0.761 | 0.995 |
| | CD3D | | | |
| 7 days | CD177 + C3AR1 + CD74 + S100A9 + CX3CR1 + CIITA + TAP2 + | 0.898 | 0.8 | 0.995 |
| | CD3D + CTLA4 | | | |
| 7 days | CD177 + C3AR1 + CD74 + S100A9 + CX3CR1 + CIITA + TAP2 + | 0.884 | 0.774 | 0.993 |
| | CTLA4 | | | |
| 7 days | CD177 + C3AR1 + CD74 + S100A9 + CX3CR1 + CTLA4 | 0.856 | 0.722 | 0.99 |
| 7 days | CD177 + C3AR1 + CD74 + S100A9 + CX3CR1 + TAP2 | 0.856 | 0.72 | 0.992 |
| 7 days | CD177 + C3AR1 + CD74 + S100A9 + CX3CR1 + TAP2 + CD3D | 0.853 | 0.719 | 0.987 |
| 7 days | CD177 + C3AR1 + CD74 + S100A9 + CX3CR1 + TAP2 + CD3D + | 0.85 | 0.715 | 0.986 |
| | CTLA4 | | | |
| 7 days | CD177 + C3AR1 + CD74 + S100A9 + CX3CR1 + TAP2 + CTLA4 | 0.859 | 0.723 | 0.994 |
| 7 days | CD177 + C3AR1 + CD74 + S100A9 + TAP2 | 0.823 | 0.68 | 0.966 |
| 7 days | CD177 + C3AR1 + CD74 + S100A9 + TAP2 + CD3D | 0.831 | 0.696 | 0.966 |
| 7 days | CD177 + C3AR1 + CD74 + S100A9 + TAP2 + CD3D + CTLA4 | 0.828 | 0.693 | 0.964 |
| 7 days | CD177 + C3AR1 + CD74 + S100A9 + TAP2 + CTLA4 | 0.823 | 0.68 | 0.966 |
| 7 days | CD177 + C3AR1 + CD74 + TAP2 | 0.82 | 0.682 | 0.958 |
| 7 days | CD177 + C3AR1 + CD74 + TAP2 + CD3D | 0.814 | 0.675 | 0.954 |
| 7 days | CD177 + C3AR1 + CD74 + TAP2 + CD3D + CTLA4 | 0.809 | 0.667 | 0.95 |
| 7 days | CD177 + C3AR1 + CD74 + TAP2 + CTLA4 | 0.817 | 0.677 | 0.958 |
| 7 days | CD177 + C3AR1 + CIITA | 0.85 | 0.725 | 0.976 |
| 7 days | CD177 + C3AR1 + CIITA + CD3D | 0.856 | 0.733 | 0.979 |
| 7 days | CD177 + C3AR1 + CIITA + CD3D + CTLA4 | 0.886 | 0.783 | 0.989 |
| 7 days | CD177 + C3AR1 + CIITA + CTLA4 | 0.875 | 0.764 | 0.987 |
| 7 days | CD177 + C3AR1 + CIITA + TAP2 | 0.85 | 0.724 | 0.977 |
| 7 days | CD177 + C3AR1 + CIITA + TAP2 + CD3D | 0.859 | 0.737 | 0.98 |
| 7 days | CD177 + C3AR1 + CIITA + TAP2 + CD3D + CTLA4 | 0.881 | 0.774 | 0.988 |
| 7 days | CD177 + C3AR1 + CIITA + TAP2 + CTLA4 | 0.875 | 0.764 | 0.987 |
| 7 days | CD177 + C3AR1 + CX3CR1 | 0.734 | 0.573 | 0.895 |
| 7 days | CD177 + C3AR1 + CX3CR1 + CD3D | 0.734 | 0.575 | 0.893 |
| 7 days | CD177 + C3AR1 + CX3CR1 + CIITA | 0.853 | 0.727 | 0.979 |
| 7 days | CD177 + C3AR1 + CX3CR1 + CIITA + CD3D | 0.853 | 0.729 | 0.977 |
| 7 days | CD177 + C3AR1 + CX3CR1 + CIITA + CD3D + CTLA4 | 0.889 | 0.786 | 0.992 |
| 7 days | CD177 + C3AR1 + CX3CR1 + CIITA + CTLA4 | 0.87 | 0.756 | 0.984 |
| 7 days | CD177 + C3AR1 + CX3CR1 + CIITA + TAP2 | 0.856 | 0.731 | 0.981 |
| 7 days | CD177 + C3AR1 + CX3CR1 + CIITA + TAP2 + CD3D | 0.853 | 0.727 | 0.979 |
| 7 days | CD177 + C3AR1 + CX3CR1 + CIITA + TAP2 + CD3D + CTLA4 | 0.892 | 0.79 | 0.994 |
| 7 days | CD177 + C3AR1 + CX3CR1 + CIITA + TAP2 + CTLA4 | 0.873 | 0.759 | 0.986 |
| 7 days | CD177 + C3AR1 + CX3CR1 + CTLA4 | 0.731 | 0.569 | 0.894 |
| 7 days | CD177 + C3AR1 + CX3CR1 + TAP2 | 0.77 | 0.614 | 0.926 |
| 7 days | CD177 + C3AR1 + CX3CR1 + TAP2 + CD3D | 0.767 | 0.613 | 0.922 |
| 7 days | CD177 + C3AR1 + CX3CR1 + TAP2 + CD3D + CTLA4 | 0.77 | 0.614 | 0.926 |
| 7 days | CD177 + C3AR1 + CX3CR1 + TAP2 + CTLA4 | 0.767 | 0.609 | 0.926 |
| 7 days | CD177 + C3AR1 + IFNG | 0.806 | 0.665 | 0.947 |
| 7 days | CD177 + C3AR1 + IFNG + CD3D | 0.812 | 0.674 | 0.949 |
| 7 days | CD177 + C3AR1 + IFNG + CD3D + CTLA4 | 0.809 | 0.671 | 0.947 |
| 7 days | CD177 + C3AR1 + IFNG + CIITA | 0.875 | 0.766 | 0.985 |
| 7 days | CD177 + C3AR1 + IFNG + CIITA + CD3D | 0.873 | 0.761 | 0.984 |
| 7 days | CD177 + C3AR1 + IFNG + CIITA + CD3D + CTLA4 | 0.881 | 0.776 | 0.985 |
| 7 days | CD177 + C3AR1 + IFNG + CIITA + CTLA4 | 0.878 | 0.774 | 0.983 |
| 7 days | CD177 + C3AR1 + IFNG + CIITA + TAP2 | 0.878 | 0.77 | 0.987 |
| 7 days | CD177 + C3AR1 + IFNG + CIITA + TAP2 + CD3D | 0.878 | 0.77 | 0.987 |
| 7 days | CD177 + C3AR1 + IFNG + CIITA + TAP2 + CD3D + CTLA4 | 0.892 | 0.795 | 0.989 |
| 7 days | CD177 + C3AR1 + IFNG + CIITA + TAP2 + CTLA4 | 0.881 | 0.778 | 0.984 |
| 7 days | CD177 + C3AR1 + IFNG + CTLA4 | 0.809 | 0.671 | 0.947 |
| 7 days | CD177 + C3AR1 + IFNG + CX3CR1 | 0.803 | 0.66 | 0.947 |
| 7 days | CD177 + C3AR1 + IFNG + CX3CR1 + CD3D | 0.814 | 0.676 | 0.953 |
| 7 days | CD177 + C3AR1 + IFNG + CX3CR1 + CD3D + CTLA4 | 0.792 | 0.648 | 0.937 |
| 7 days | CD177 + C3AR1 + IFNG + CX3CR1 + CIITA | 0.884 | 0.78 | 0.987 |
| 7 days | CD177 + C3AR1 + IFNG + CX3CR1 + CIITA + CD3D | 0.881 | 0.775 | 0.987 |
| 7 days | CD177 + C3AR1 + IFNG + CX3CR1 + CIITA + CD3D + CTLA4 | 0.9 | 0.805 | 0.996 |
| 7 days | CD177 + C3AR1 + IFNG + CX3CR1 + CIITA + CTLA4 | 0.889 | 0.79 | 0.988 |
| 7 days | CD177 + C3AR1 + IFNG + CX3CR1 + CIITA + TAP2 | 0.889 | 0.79 | 0.988 |
| 7 days | CD177 + C3AR1 + IFNG + CX3CR1 + CIITA + TAP2 + CD3D | 0.884 | 0.781 | 0.987 |
| 7 days | CD177 + C3AR1 + IFNG + CX3CR1 + CIITA + TAP2 + CD3D + | 0.9 | 0.803 | 0.997 |
| | CTLA4 | | | |
| 7 days | CD177 + C3AR1 + IFNG + CX3CR1 + CIITA + TAP2 + CTLA4 | 0.886 | 0.784 | 0.989 |
| 7 days | CD177 + C3AR1 + IFNG + CX3CR1 + CTLA4 | 0.801 | 0.659 | 0.942 |
| 7 days | CD177 + C3AR1 + IFNG + CX3CR1 + TAP2 | 0.817 | 0.68 | 0.954 |
| 7 days | CD177 + C3AR1 + IFNG + CX3CR1 + TAP2 + CD3D | 0.82 | 0.684 | 0.956 |
| 7 days | CD177 + C3AR1 + IFNG + CX3CR1 + TAP2 + CD3D + CTLA4 | 0.825 | 0.693 | 0.958 |
| 7 days | CD177 + C3AR1 + IFNG + CX3CR1 + TAP2 + CTLA4 | 0.825 | 0.693 | 0.958 |
| 7 days | CD177 + C3AR1 + IFNG + S100A9 | 0.798 | 0.655 | 0.94 |
| 7 days | CD177 + C3AR1 + IFNG + S100A9 + CD3D | 0.795 | 0.651 | 0.939 |
| 7 days | CD177 + C3AR1 + IFNG + S100A9 + CD3D + CTLA4 | 0.801 | 0.659 | 0.942 |
| 7 days | CD177 + C3AR1 + IFNG + S100A9 + CIITA | 0.873 | 0.762 | 0.983 |
| 7 days | CD177 + C3AR1 + IFNG + S100A9 + CIITA + CD3D | 0.875 | 0.766 | 0.985 |
| 7 days | CD177 + C3AR1 + IFNG + S100A9 + CIITA + CD3D + CTLA4 | 0.881 | 0.777 | 0.985 |
| 7 days | CD177 + C3AR1 + IFNG + S100A9 + CIITA + CTLA4 | 0.873 | 0.765 | 0.98 |
| 7 days | CD177 + C3AR1 + IFNG + S100A9 + CIITA + TAP2 | 0.873 | 0.763 | 0.982 |
| 7 days | CD177 + C3AR1 + IFNG + S100A9 + CIITA + TAP2 + CD3D | 0.873 | 0.762 | 0.983 |
| 7 days | CD177 + C3AR1 + IFNG + S100A9 + CIITA + TAP2 + CD3D + | 0.892 | 0.795 | 0.989 |
| | CTLA4 | | | |
| 7 days | CD177 + C3AR1 + IFNG + S100A9 + CIITA + TAP2 + CTLA4 | 0.881 | 0.777 | 0.985 |
| 7 days | CD177 + C3AR1 + IFNG + S100A9 + CTLA4 | 0.801 | 0.659 | 0.942 |
| 7 days | CD177 + C3AR1 + IFNG + S100A9 + CX3CR1 | 0.795 | 0.652 | 0.938 |
| 7 days | CD177 + C3AR1 + IFNG + S100A9 + CX3CR1 + CD3D | 0.789 | 0.646 | 0.933 |
| 7 days | CD177 + C3AR1 + IFNG + S100A9 + CX3CR1 + CD3D + CTLA4 | 0.789 | 0.646 | 0.933 |
| 7 days | CD177 + C3AR1 + IFNG + S100A9 + CX3CR1 + CIITA | 0.884 | 0.778 | 0.989 |
| 7 days | CD177 + C3AR1 + IFNG + S100A9 + CX3CR1 + CIITA + CD3D | 0.881 | 0.775 | 0.987 |
| 7 days | CD177 + C3AR1 + IFNG + S100A9 + CX3CR1 + CIITA + CD3D + | 0.895 | 0.797 | 0.993 |
| | CTLA4 | | | |
| 7 days | CD177 + C3AR1 + IFNG + S100A9 + CX3CR1 + CIITA + CTLA4 | 0.889 | 0.788 | 0.991 |
| 7 days | CD177 + C3AR1 + IFNG + S100A9 + CX3CR1 + CIITA + TAP2 | 0.903 | 0.807 | 0.999 |
| 7 days | CD177 + C3AR1 + IFNG + S100A9 + CX3CR1 + CIITA + TAP2 + | 0.9 | 0.803 | 0.997 |
| | CD3D | | | |
| 7 days | CD177 + C3AR1 + IFNG + S100A9 + CX3CR1 + CIITA + TAP2 + | 0.892 | 0.792 | 0.992 |
| | CD3D + CTLA4 | | | |
| 7 days | CD177 + C3AR1 + IFNG + S100A9 + CX3CR1 + CIITA + TAP2 + | 0.892 | 0.789 | 0.995 |
| | CTLA4 | | | |
| 7 days | CD177 + C3AR1 + IFNG + S100A9 + CX3CR1 + CTLA4 | 0.806 | 0.667 | 0.945 |
| 7 days | CD177 + C3AR1 + IFNG + S100A9 + CX3CR1 + TAP2 | 0.82 | 0.688 | 0.952 |
| 7 days | CD177 + C3AR1 + IFNG + S100A9 + CX3CR1 + TAP2 + CD3D | 0.809 | 0.672 | 0.945 |
| 7 days | CD177 + C3AR1 + IFNG + S100A9 + CX3CR1 + TAP2 + CD3D + | 0.817 | 0.684 | 0.951 |
| | CTLA4 | | | |
| 7 days | CD177 + C3AR1 + IFNG + S100A9 + CX3CR1 + TAP2 + CTLA4 | 0.823 | 0.691 | 0.954 |
| 7 days | CD177 + C3AR1 + IFNG + S100A9 + TAP2 | 0.801 | 0.66 | 0.941 |
| 7 days | CD177 + C3AR1 + IFNG + S100A9 + TAP2 + CD3D | 0.798 | 0.656 | 0.94 |
| 7 days | CD177 + C3AR1 + IFNG + S100A9 + TAP2 + CD3D + CTLA4 | 0.806 | 0.667 | 0.945 |
| 7 days | CD177 + C3AR1 + IFNG + S100A9 + TAP2 + CTLA4 | 0.798 | 0.656 | 0.94 |
| 7 days | CD177 + C3AR1 + IFNG + TAP2 | 0.809 | 0.669 | 0.949 |
| 7 days | CD177 + C3AR1 + IFNG + TAP2 + CD3D | 0.812 | 0.673 | 0.95 |
| 7 days | CD177 + C3AR1 + IFNG + TAP2 + CD3D + CTLA4 | 0.806 | 0.666 | 0.947 |
| 7 days | CD177 + C3AR1 + IFNG + TAP2 + CTLA4 | 0.812 | 0.673 | 0.95 |
| 7 days | CD177 + C3AR1 + S100A9 | 0.762 | 0.605 | 0.918 |
| 7 days | CD177 + C3AR1 + S100A9 + CD3D | 0.767 | 0.615 | 0.92 |
| 7 days | CD177 + C3AR1 + S100A9 + CD3D + CTLA4 | 0.756 | 0.602 | 0.91 |
| 7 days | CD177 + C3AR1 + S100A9 + CIITA | 0.853 | 0.729 | 0.977 |
| 7 days | CD177 + C3AR1 + S100A9 + CIITA + CD3D | 0.859 | 0.736 | 0.981 |
| 7 days | CD177 + C3AR1 + S100A9 + CIITA + CD3D + CTLA4 | 0.878 | 0.77 | 0.986 |
| 7 days | CD177 + C3AR1 + S100A9 + CIITA + CTLA4 | 0.875 | 0.764 | 0.987 |
| 7 days | CD177 + C3AR1 + S100A9 + CIITA + TAP2 | 0.856 | 0.734 | 0.978 |
| 7 days | CD177 + C3AR1 + S100A9 + CIITA + TAP2 + CD3D | 0.859 | 0.736 | 0.981 |
| 7 days | CD177 + C3AR1 + S100A9 + CIITA + TAP2 + CD3D + CTLA4 | 0.886 | 0.783 | 0.99 |
| 7 days | CD177 + C3AR1 + S100A9 + CIITA + TAP2 + CTLA4 | 0.878 | 0.768 | 0.988 |
| 7 days | CD177 + C3AR1 + S100A9 + CTLA4 | 0.765 | 0.608 | 0.921 |
| 7 days | CD177 + C3AR1 + S100A9 + CX3CR1 | 0.77 | 0.616 | 0.924 |
| 7 days | CD177 + C3AR1 + S100A9 + CX3CR1 + CD3D | 0.784 | 0.636 | 0.932 |
| 7 days | CD177 + C3AR1 + S100A9 + CX3CR1 + CD3D + CTLA4 | 0.773 | 0.621 | 0.925 |
| 7 days | CD177 + C3AR1 + S100A9 + CX3CR1 + CIITA | 0.856 | 0.733 | 0.979 |
| 7 days | CD177 + C3AR1 + S100A9 + CX3CR1 + CIITA + CD3D | 0.867 | 0.747 | 0.987 |
| 7 days | CD177 + C3AR1 + S100A9 + CX3CR1 + CIITA + CD3D + CTLA4 | 0.898 | 0.799 | 0.996 |
| 7 days | CD177 + C3AR1 + S100A9 + CX3CR1 + CIITA + CTLA4 | 0.873 | 0.761 | 0.985 |
| 7 days | CD177 + C3AR1 + S100A9 + CX3CR1 + CIITA + TAP2 | 0.859 | 0.735 | 0.982 |
| 7 days | CD177 + C3AR1 + S100A9 + CX3CR1 + CIITA + TAP2 + CD3D | 0.856 | 0.733 | 0.979 |
| 7 days | CD177 + C3AR1 + S100A9 + CX3CR1 + CIITA + TAP2 + CD3D + | 0.898 | 0.799 | 0.996 |
| | CTLA4 | | | |
| 7 days | CD177 + C3AR1 + S100A9 + CX3CR1 + CIITA + TAP2 + CTLA4 | 0.881 | 0.774 | 0.988 |
| 7 days | CD177 + C3AR1 + S100A9 + CX3CR1 + CTLA4 | 0.77 | 0.616 | 0.924 |
| 7 days | CD177 + C3AR1 + S100A9 + CX3CR1 + TAP2 | 0.781 | 0.629 | 0.933 |
| 7 days | CD177 + C3AR1 + S100A9 + CX3CR1 + TAP2 + CD3D | 0.781 | 0.631 | 0.932 |
| 7 days | CD177 + C3AR1 + S100A9 + CX3CR1 + TAP2 + CD3D + CTLA4 | 0.784 | 0.637 | 0.931 |
| 7 days | CD177 + C3AR1 + S100A9 + CX3CR1 + TAP2 + CTLA4 | 0.789 | 0.64 | 0.939 |
| 7 days | CD177 + C3AR1 + S100A9 + TAP2 | 0.792 | 0.643 | 0.941 |
| 7 days | CD177 + C3AR1 + S100A9 + TAP2 + CD3D | 0.789 | 0.644 | 0.935 |
| 7 days | CD177 + C3AR1 + S100A9 + TAP2 + CD3D + CTLA4 | 0.789 | 0.644 | 0.935 |
| 7 days | CD177 + C3AR1 + S100A9 + TAP2 + CTLA4 | 0.792 | 0.643 | 0.941 |
| 7 days | CD177 + C3AR1 + TAP2 | 0.767 | 0.611 | 0.924 |
| 7 days | CD177 + C3AR1 + TAP2 + CD3D | 0.762 | 0.606 | 0.918 |
| 7 days | CD177 + C3AR1 + TAP2 + CD3D + CTLA4 | 0.77 | 0.615 | 0.925 |
| 7 days | CD177 + C3AR1 + TAP2 + CTLA4 | 0.767 | 0.612 | 0.923 |
| 7 days | CD177 + CD74 | 0.77 | 0.612 | 0.928 |
| 7 days | CD177 + CD74 + CD3D | 0.767 | 0.609 | 0.926 |
| 7 days | CD177 + CD74 + CD3D + CTLA4 | 0.767 | 0.606 | 0.928 |
| 7 days | CD177 + CD74 + CIITA | 0.798 | 0.647 | 0.949 |
| 7 days | CD177 + CD74 + CIITA + CD3D | 0.803 | 0.658 | 0.949 |
| 7 days | CD177 + CD74 + CIITA + CD3D + CTLA4 | 0.787 | 0.636 | 0.938 |
| 7 days | CD177 + CD74 + CIITA + CTLA4 | 0.798 | 0.651 | 0.945 |
| 7 days | CD177 + CD74 + CIITA + TAP2 | 0.789 | 0.64 | 0.939 |
| 7 days | CD177 + CD74 + CIITA + TAP2 + CD3D | 0.806 | 0.662 | 0.951 |
| 7 days | CD177 + CD74 + CIITA + TAP2 + CD3D + CTLA4 | 0.787 | 0.636 | 0.938 |
| 7 days | CD177 + CD74 + CIITA + TAP2 + CTLA4 | 0.792 | 0.644 | 0.941 |
| 7 days | CD177 + CD74 + CTLA4 | 0.759 | 0.598 | 0.92 |
| 7 days | CD177 + CD74 + CX3CR1 | 0.778 | 0.618 | 0.939 |
| 7 days | CD177 + CD74 + CX3CR1 + CD3D | 0.773 | 0.612 | 0.934 |
| 7 days | CD177 + CD74 + CX3CR1 + CD3D + CTLA4 | 0.77 | 0.609 | 0.931 |
| 7 days | CD177 + CD74 + CX3CR1 + CIITA | 0.839 | 0.702 | 0.977 |
| 7 days | CD177 + CD74 + CX3CR1 + CIITA + CD3D | 0.825 | 0.685 | 0.966 |
| 7 days | CD177 + CD74 + CX3CR1 + CIITA + CD3D + CTLA4 | 0.814 | 0.671 | 0.958 |
| 7 days | CD177 + CD74 + CX3CR1 + CIITA + CTLA4 | 0.828 | 0.686 | 0.97 |
| 7 days | CD177 + CD74 + CX3CR1 + CIITA + TAP2 | 0.823 | 0.678 | 0.968 |
| 7 days | CD177 + CD74 + CX3CR1 + CIITA + TAP2 + CD3D | 0.82 | 0.676 | 0.964 |
| 7 days | CD177 + CD74 + CX3CR1 + CIITA + TAP2 + CD3D + CTLA4 | 0.823 | 0.678 | 0.968 |
| 7 days | CD177 + CD74 + CX3CR1 + CIITA + TAP2 + CTLA4 | 0.82 | 0.673 | 0.967 |
| 7 days | CD177 + CD74 + CX3CR1 + CTLA4 | 0.784 | 0.626 | 0.942 |
| 7 days | CD177 + CD74 + CX3CR1 + TAP2 | 0.784 | 0.627 | 0.941 |
| 7 days | CD177 + CD74 + CX3CR1 + TAP2 + CD3D | 0.787 | 0.631 | 0.942 |
| 7 days | CD177 + CD74 + CX3CR1 + TAP2 + CD3D + CTLA4 | 0.784 | 0.626 | 0.942 |
| 7 days | CD177 + CD74 + CX3CR1 + TAP2 + CTLA4 | 0.784 | 0.627 | 0.941 |
| 7 days | CD177 + CD74 + IFNG | 0.773 | 0.618 | 0.928 |
| 7 days | CD177 + CD74 + IFNG + CD3D | 0.773 | 0.615 | 0.931 |
| 7 days | CD177 + CD74 + IFNG + CD3D + CTLA4 | 0.789 | 0.636 | 0.943 |
| 7 days | CD177 + CD74 + IFNG + CIITA | 0.789 | 0.638 | 0.941 |
| 7 days | CD177 + CD74 + IFNG + CIITA + CD3D | 0.795 | 0.646 | 0.944 |
| 7 days | CD177 + CD74 + IFNG + CIITA + CD3D + CTLA4 | 0.795 | 0.647 | 0.943 |
| 7 days | CD177 + CD74 + IFNG + CIITA + CTLA4 | 0.798 | 0.649 | 0.947 |
| 7 days | CD177 + CD74 + IFNG + CIITA + TAP2 | 0.801 | 0.653 | 0.948 |
| 7 days | CD177 + CD74 + IFNG + CIITA + TAP2 + CD3D | 0.798 | 0.645 | 0.95 |
| 7 days | CD177 + CD74 + IFNG + CIITA + TAP2 + CD3D + CTLA4 | 0.803 | 0.655 | 0.952 |
| 7 days | CD177 + CD74 + IFNG + CIITA + TAP2 + CTLA4 | 0.806 | 0.656 | 0.956 |
| 7 days | CD177 + CD74 + IFNG + CTLA4 | 0.776 | 0.62 | 0.931 |
| 7 days | CD177 + CD74 + IFNG + CX3CR1 | 0.837 | 0.708 | 0.965 |
| 7 days | CD177 + CD74 + IFNG + CX3CR1 + CD3D | 0.839 | 0.712 | 0.967 |
| 7 days | CD177 + CD74 + IFNG + CX3CR1 + CD3D + CTLA4 | 0.845 | 0.721 | 0.969 |
| 7 days | CD177 + CD74 + IFNG + CX3CR1 + CIITA | 0.884 | 0.778 | 0.989 |
| 7 days | CD177 + CD74 + IFNG + CX3CR1 + CIITA + CD3D | 0.878 | 0.771 | 0.986 |
| 7 days | CD177 + CD74 + IFNG + CX3CR1 + CIITA + CD3D + CTLA4 | 0.878 | 0.769 | 0.987 |
| 7 days | CD177 + CD74 + IFNG + CX3CR1 + CIITA + CTLA4 | 0.881 | 0.775 | 0.987 |
| 7 days | CD177 + CD74 + IFNG + CX3CR1 + CIITA + TAP2 | 0.881 | 0.774 | 0.988 |
| 7 days | CD177 + CD74 + IFNG + CX3CR1 + CIITA + TAP2 + CD3D | 0.875 | 0.766 | 0.985 |
| 7 days | CD177 + CD74 + IFNG + CX3CR1 + CIITA + TAP2 + CD3D + | 0.881 | 0.775 | 0.987 |
| | CTLA4 | | | |
| 7 days | CD177 + CD74 + IFNG + CX3CR1 + CIITA + TAP2 + CTLA4 | 0.884 | 0.78 | 0.987 |
| 7 days | CD177 + CD74 + IFNG + CX3CR1 + CTLA4 | 0.85 | 0.729 | 0.971 |
| 7 days | CD177 + CD74 + IFNG + CX3CR1 + TAP2 | 0.845 | 0.72 | 0.97 |
| 7 days | CD177 + CD74 + IFNG + CX3CR1 + TAP2 + CD3D | 0.839 | 0.712 | 0.967 |
| 7 days | CD177 + CD74 + IFNG + CX3CR1 + TAP2 + CD3D + CTLA4 | 0.845 | 0.72 | 0.97 |
| 7 days | CD177 + CD74 + IFNG + CX3CR1 + TAP2 + CTLA4 | 0.85 | 0.729 | 0.972 |
| 7 days | CD177 + CD74 + IFNG + S100A9 | 0.77 | 0.614 | 0.926 |
| 7 days | CD177 + CD74 + IFNG + S100A9 + CD3D | 0.765 | 0.605 | 0.924 |
| 7 days | CD177 + CD74 + IFNG + S100A9 + CD3D + CTLA4 | 0.784 | 0.631 | 0.937 |
| 7 days | CD177 + CD74 + IFNG + S100A9 + CIITA | 0.803 | 0.654 | 0.953 |
| 7 days | CD177 + CD74 + IFNG + S100A9 + CIITA + CD3D | 0.795 | 0.649 | 0.941 |
| 7 days | CD177 + CD74 + IFNG + S100A9 + CIITA + CD3D + CTLA4 | 0.803 | 0.661 | 0.946 |
| 7 days | CD177 + CD74 + IFNG + S100A9 + CIITA + CTLA4 | 0.803 | 0.656 | 0.951 |
| 7 days | CD177 + CD74 + IFNG + S100A9 + CIITA + TAP2 | 0.787 | 0.637 | 0.936 |
| 7 days | CD177 + CD74 + IFNG + S100A9 + CIITA + TAP2 + CD3D | 0.806 | 0.665 | 0.948 |
| 7 days | CD177 + CD74 + IFNG + S100A9 + CIITA + TAP2 + CD3D + | 0.803 | 0.661 | 0.945 |
| | CTLA4 | | | |
| 7 days | CD177 + CD74 + IFNG + S100A9 + CIITA + TAP2 + CTLA4 | 0.803 | 0.658 | 0.949 |
| 7 days | CD177 + CD74 + IFNG + S100A9 + CTLA4 | 0.773 | 0.617 | 0.929 |
| 7 days | CD177 + CD74 + IFNG + S100A9 + CX3CR1 | 0.839 | 0.712 | 0.967 |
| 7 days | CD177 + CD74 + IFNG + S100A9 + CX3CR1 + CD3D | 0.842 | 0.716 | 0.968 |
| 7 days | CD177 + CD74 + IFNG + S100A9 + CX3CR1 + CD3D + CTLA4 | 0.845 | 0.721 | 0.969 |
| 7 days | CD177 + CD74 + IFNG + S100A9 + CX3CR1 + CIITA | 0.886 | 0.782 | 0.991 |
| 7 days | CD177 + CD74 + IFNG + S100A9 + CX3CR1 + CIITA + CD3D | 0.875 | 0.765 | 0.986 |
| 7 days | CD177 + CD74 + IFNG + S100A9 + CX3CR1 + CIITA + CD3D + | 0.878 | 0.769 | 0.987 |
| | CTLA4 | | | |
| 7 days | CD177 + CD74 + IFNG + S100A9 + CX3CR1 + CIITA + CTLA4 | 0.889 | 0.785 | 0.993 |
| 7 days | CD177 + CD74 + IFNG + S100A9 + CX3CR1 + CIITA + TAP2 | 0.884 | 0.776 | 0.991 |
| 7 days | CD177 + CD74 + IFNG + S100A9 + CX3CR1 + CIITA + TAP2 + | 0.884 | 0.778 | 0.989 |
| | CD3D | | | |
| 7 days | CD177 + CD74 + IFNG + S100A9 + CX3CR1 + CIITA + TAP2 + | 0.884 | 0.778 | 0.989 |
| | CD3D + CTLA4 | | | |
| 7 days | CD177 + CD74 + IFNG + S100A9 + CX3CR1 + CIITA + TAP2 + | 0.892 | 0.789 | 0.995 |
| | CTLA4 | | | |
| 7 days | CD177 + CD74 + IFNG + S100A9 + CX3CR1 + CTLA4 | 0.848 | 0.724 | 0.971 |
| 7 days | CD177 + CD74 + IFNG + S100A9 + CX3CR1 + TAP2 | 0.839 | 0.712 | 0.967 |
| 7 days | CD177 + CD74 + IFNG + S100A9 + CX3CR1 + TAP2 + CD3D | 0.839 | 0.712 | 0.967 |
| 7 days | CD177 + CD74 + IFNG + S100A9 + CX3CR1 + TAP2 + CD3D + | 0.845 | 0.72 | 0.97 |
| | CTLA4 | | | |
| 7 days | CD177 + CD74 + IFNG + S100A9 + CX3CR1 + TAP2 + CTLA4 | 0.842 | 0.715 | 0.969 |
| 7 days | CD177 + CD74 + IFNG + S100A9 + TAP2 | 0.762 | 0.602 | 0.922 |
| 7 days | CD177 + CD74 + IFNG + S100A9 + TAP2 + CD3D | 0.77 | 0.611 | 0.929 |
| 7 days | CD177 + CD74 + IFNG + S100A9 + TAP2 + CD3D + CTLA4 | 0.773 | 0.617 | 0.928 |
| 7 days | CD177 + CD74 + IFNG + S100A9 + TAP2 + CTLA4 | 0.756 | 0.596 | 0.917 |
| 7 days | CD177 + CD74 + IFNG + TAP2 | 0.767 | 0.61 | 0.925 |
| 7 days | CD177 + CD74 + IFNG + TAP2 + CD3D | 0.773 | 0.615 | 0.931 |
| 7 days | CD177 + CD74 + IFNG + TAP2 + CD3D + CTLA4 | 0.784 | 0.63 | 0.937 |
| 7 days | CD177 + CD74 + IFNG + TAP2 + CTLA4 | 0.77 | 0.614 | 0.926 |
| 7 days | CD177 + CD74 + S100A9 | 0.781 | 0.624 | 0.938 |
| 7 days | CD177 + CD74 + S100A9 + CD3D | 0.784 | 0.627 | 0.941 |
| 7 days | CD177 + CD74 + S100A9 + CD3D + CTLA4 | 0.778 | 0.621 | 0.936 |
| 7 days | CD177 + CD74 + S100A9 + CIITA | 0.795 | 0.645 | 0.945 |
| 7 days | CD177 + CD74 + S100A9 + CIITA + CD3D | 0.792 | 0.644 | 0.941 |
| 7 days | CD177 + CD74 + S100A9 + CIITA + CD3D + CTLA4 | 0.789 | 0.639 | 0.94 |
| 7 days | CD177 + CD74 + S100A9 + CIITA + CTLA4 | 0.798 | 0.65 | 0.945 |
| 7 days | CD177 + CD74 + S100A9 + CIITA + TAP2 | 0.792 | 0.643 | 0.941 |
| 7 days | CD177 + CD74 + S100A9 + CIITA + TAP2 + CD3D | 0.789 | 0.641 | 0.938 |
| 7 days | CD177 + CD74 + S100A9 + CIITA + TAP2 + CD3D + CTLA4 | 0.787 | 0.636 | 0.937 |
| 7 days | CD177 + CD74 + S100A9 + CIITA + TAP2 + CTLA4 | 0.795 | 0.647 | 0.943 |
| 7 days | CD177 + CD74 + S100A9 + CTLA4 | 0.781 | 0.626 | 0.937 |
| 7 days | CD177 + CD74 + S100A9 + CX3CR1 | 0.817 | 0.657 | 0.977 |
| 7 days | CD177 + CD74 + S100A9 + CX3CR1 + CD3D | 0.817 | 0.663 | 0.972 |
| 7 days | CD177 + CD74 + S100A9 + CX3CR1 + CD3D + CTLA4 | 0.817 | 0.661 | 0.973 |
| 7 days | CD177 + CD74 + S100A9 + CX3CR1 + CIITA | 0.834 | 0.694 | 0.974 |
| 7 days | CD177 + CD74 + S100A9 + CX3CR1 + CIITA + CD3D | 0.834 | 0.694 | 0.973 |
| 7 days | CD177 + CD74 + S100A9 + CX3CR1 + CIITA + CD3D + CTLA4 | 0.839 | 0.706 | 0.973 |
| 7 days | CD177 + CD74 + S100A9 + CX3CR1 + CIITA + CTLA4 | 0.828 | 0.686 | 0.971 |
| 7 days | CD177 + CD74 + S100A9 + CX3CR1 + CIITA + TAP2 | 0.825 | 0.683 | 0.968 |
| 7 days | CD177 + CD74 + S100A9 + CX3CR1 + CIITA + TAP2 + CD3D | 0.823 | 0.68 | 0.966 |
| 7 days | CD177 + CD74 + S100A9 + CX3CR1 + CIITA + TAP2 + CD3D + | 0.831 | 0.693 | 0.969 |
| | CTLA4 | | | |
| 7 days | CD177 + CD74 + S100A9 + CX3CR1 + CIITA + TAP2 + CTLA4 | 0.812 | 0.661 | 0.962 |
| 7 days | CD177 + CD74 + S100A9 + CX3CR1 + CTLA4 | 0.828 | 0.672 | 0.985 |
| 7 days | CD177 + CD74 + S100A9 + CX3CR1 + TAP2 | 0.825 | 0.67 | 0.981 |
| 7 days | CD177 + CD74 + S100A9 + CX3CR1 + TAP2 + CD3D | 0.812 | 0.656 | 0.968 |
| 7 days | CD177 + CD74 + S100A9 + CX3CR1 + TAP2 + CD3D + CTLA4 | 0.814 | 0.658 | 0.97 |
| 7 days | CD177 + CD74 + S100A9 + CX3CR1 + TAP2 + CTLA4 | 0.817 | 0.663 | 0.972 |
| 7 days | CD177 + CD74 + S100A9 + TAP2 | 0.784 | 0.627 | 0.941 |
| 7 days | CD177 + CD74 + S100A9 + TAP2 + CD3D | 0.784 | 0.627 | 0.941 |
| 7 days | CD177 + CD74 + S100A9 + TAP2 + CD3D + CTLA4 | 0.781 | 0.624 | 0.938 |
| 7 days | CD177 + CD74 + S100A9 + TAP2 + CTLA4 | 0.776 | 0.619 | 0.932 |
| 7 days | CD177 + CD74 + TAP2 | 0.773 | 0.616 | 0.93 |
| 7 days | CD177 + CD74 + TAP2 + CD3D | 0.773 | 0.616 | 0.93 |
| 7 days | CD177 + CD74 + TAP2 + CD3D + CTLA4 | 0.765 | 0.603 | 0.926 |
| 7 days | CD177 + CD74 + TAP2 + CTLA4 | 0.759 | 0.598 | 0.92 |
| 7 days | CD177 + CIITA + CD3D | 0.798 | 0.651 | 0.944 |
| 7 days | CD177 + CIITA + CD3D + CTLA4 | 0.795 | 0.647 | 0.943 |
| 7 days | CD177 + CIITA + CTLA4 | 0.803 | 0.657 | 0.949 |
| 7 days | CD177 + CIITA + TAP2 | 0.806 | 0.656 | 0.956 |
| 7 days | CD177 + CIITA + TAP2 + CD3D | 0.803 | 0.658 | 0.949 |
| 7 days | CD177 + CIITA + TAP2 + CD3D + CTLA4 | 0.795 | 0.647 | 0.943 |
| 7 days | CD177 + CIITA + TAP2 + CTLA4 | 0.806 | 0.662 | 0.95 |
| 7 days | CD177 + CX3CR1 + CD3D + CTLA4 | 0.737 | 0.573 | 0.901 |
| 7 days | CD177 + CX3CR1 + CIITA | 0.823 | 0.68 | 0.965 |
| 7 days | CD177 + CX3CR1 + CIITA + CD3D | 0.825 | 0.684 | 0.967 |
| 7 days | CD177 + CX3CR1 + CIITA + CD3D + CTLA4 | 0.817 | 0.678 | 0.956 |
| 7 days | CD177 + CX3CR1 + CIITA + CTLA4 | 0.823 | 0.682 | 0.963 |
| 7 days | CD177 + CX3CR1 + CIITA + TAP2 | 0.823 | 0.679 | 0.967 |
| 7 days | CD177 + CX3CR1 + CIITA + TAP2 + CD3D | 0.814 | 0.67 | 0.959 |
| 7 days | CD177 + CX3CR1 + CIITA + TAP2 + CD3D + CTLA4 | 0.825 | 0.687 | 0.964 |
| 7 days | CD177 + CX3CR1 + CIITA + TAP2 + CTLA4 | 0.828 | 0.687 | 0.969 |
| 7 days | CD177 + CX3CR1 + TAP2 + CD3D + CTLA4 | 0.731 | 0.562 | 0.9 |
| 7 days | CD177 + CX3CR1 + TAP2 + CTLA4 | 0.731 | 0.563 | 0.9 |
| 7 days | CD177 + IFNG + CD3D | 0.765 | 0.607 | 0.922 |
| 7 days | CD177 + IFNG + CD3D + CTLA4 | 0.751 | 0.59 | 0.911 |
| 7 days | CD177 + IFNG + CIITA | 0.812 | 0.668 | 0.955 |
| 7 days | CD177 + IFNG + CIITA + CD3D | 0.789 | 0.64 | 0.939 |
| 7 days | CD177 + IFNG + CIITA + CD3D + CTLA4 | 0.801 | 0.654 | 0.947 |
| 7 days | CD177 + IFNG + CIITA + CTLA4 | 0.812 | 0.668 | 0.955 |
| 7 days | CD177 + IFNG + CIITA + TAP2 | 0.806 | 0.661 | 0.951 |
| 7 days | CD177 + IFNG + CIITA + TAP2 + CD3D | 0.789 | 0.639 | 0.94 |
| 7 days | CD177 + IFNG + CIITA + TAP2 + CD3D + CTLA4 | 0.809 | 0.665 | 0.953 |
| 7 days | CD177 + IFNG + CIITA + TAP2 + CTLA4 | 0.812 | 0.668 | 0.956 |
| 7 days | CD177 + IFNG + CTLA4 | 0.751 | 0.591 | 0.91 |
| 7 days | CD177 + IFNG + CX3CR1 | 0.776 | 0.617 | 0.934 |
| 7 days | CD177 + IFNG + CX3CR1 + CD3D | 0.781 | 0.625 | 0.937 |
| 7 days | CD177 + IFNG + CX3CR1 + CD3D + CTLA4 | 0.792 | 0.64 | 0.945 |
| 7 days | CD177 + IFNG + CX3CR1 + CIITA | 0.85 | 0.719 | 0.981 |
| 7 days | CD177 + IFNG + CX3CR1 + CIITA + CD3D | 0.845 | 0.712 | 0.978 |
| 7 days | CD177 + IFNG + CX3CR1 + CIITA + CD3D + CTLA4 | 0.859 | 0.733 | 0.985 |
| 7 days | CD177 + IFNG + CX3CR1 + CIITA + CTLA4 | 0.853 | 0.724 | 0.982 |
| 7 days | CD177 + IFNG + CX3CR1 + CIITA + TAP2 | 0.842 | 0.714 | 0.97 |
| 7 days | CD177 + IFNG + CX3CR1 + CIITA + TAP2 + CD3D | 0.842 | 0.714 | 0.97 |
| 7 days | CD177 + IFNG + CX3CR1 + CIITA + TAP2 + CD3D + CTLA4 | 0.853 | 0.728 | 0.978 |
| 7 days | CD177 + IFNG + CX3CR1 + CIITA + TAP2 + CTLA4 | 0.845 | 0.718 | 0.972 |
| 7 days | CD177 + IFNG + CX3CR1 + CTLA4 | 0.792 | 0.641 | 0.944 |
| 7 days | CD177 + IFNG + CX3CR1 + TAP2 | 0.784 | 0.632 | 0.935 |
| 7 days | CD177 + IFNG + CX3CR1 + TAP2 + CD3D | 0.787 | 0.637 | 0.937 |
| 7 days | CD177 + IFNG + CX3CR1 + TAP2 + CD3D + CTLA4 | 0.801 | 0.658 | 0.943 |
| 7 days | CD177 + IFNG + CX3CR1 + TAP2 + CTLA4 | 0.792 | 0.647 | 0.937 |
| 7 days | CD177 + IFNG + S100A9 | 0.748 | 0.587 | 0.909 |
| 7 days | CD177 + IFNG + S100A9 + CD3D | 0.759 | 0.601 | 0.917 |
| 7 days | CD177 + IFNG + S100A9 + CD3D + CTLA4 | 0.753 | 0.594 | 0.913 |
| 7 days | CD177 + IFNG + S100A9 + CIITA | 0.812 | 0.666 | 0.958 |
| 7 days | CD177 + IFNG + S100A9 + CIITA + CD3D | 0.801 | 0.657 | 0.945 |
| 7 days | CD177 + IFNG + S100A9 + CIITA + CD3D + CTLA4 | 0.798 | 0.653 | 0.943 |
| 7 days | CD177 + IFNG + S100A9 + CIITA + CTLA4 | 0.809 | 0.665 | 0.953 |
| 7 days | CD177 + IFNG + S100A9 + CIITA + TAP2 | 0.809 | 0.663 | 0.955 |
| 7 days | CD177 + IFNG + S100A9 + CIITA + TAP2 + CD3D | 0.795 | 0.65 | 0.94 |
| 7 days | CD177 + IFNG + S100A9 + CIITA + TAP2 + CD3D + CTLA4 | 0.798 | 0.653 | 0.943 |
| 7 days | CD177 + IFNG + S100A9 + CIITA + TAP2 + CTLA4 | 0.806 | 0.661 | 0.951 |
| 7 days | CD177 + IFNG + S100A9 + CTLA4 | 0.753 | 0.594 | 0.913 |
| 7 days | CD177 + IFNG + S100A9 + CX3CR1 | 0.776 | 0.623 | 0.928 |
| 7 days | CD177 + IFNG + S100A9 + CX3CR1 + CD3D | 0.798 | 0.651 | 0.945 |
| 7 days | CD177 + IFNG + S100A9 + CX3CR1 + CD3D + CTLA4 | 0.801 | 0.652 | 0.949 |
| 7 days | CD177 + IFNG + S100A9 + CX3CR1 + CIITA | 0.853 | 0.727 | 0.979 |
| 7 days | CD177 + IFNG + S100A9 + CX3CR1 + CIITA + CD3D | 0.853 | 0.727 | 0.979 |
| 7 days | CD177 + IFNG + S100A9 + CX3CR1 + CIITA + CD3D + CTLA4 | 0.853 | 0.725 | 0.982 |
| 7 days | CD177 + IFNG + S100A9 + CX3CR1 + CIITA + CTLA4 | 0.85 | 0.721 | 0.98 |
| 7 days | CD177 + IFNG + S100A9 + CX3CR1 + CIITA + TAP2 | 0.848 | 0.72 | 0.975 |
| 7 days | CD177 + IFNG + S100A9 + CX3CR1 + CIITA + TAP2 + CD3D | 0.848 | 0.72 | 0.975 |
| 7 days | CD177 + IFNG + S100A9 + CX3CR1 + CIITA + TAP2 + CD3D + | 0.845 | 0.716 | 0.974 |
| | CTLA4 | | | |
| 7 days | CD177 + IFNG + S100A9 + CX3CR1 + CIITA + TAP2 + CTLA4 | 0.842 | 0.712 | 0.972 |
| 7 days | CD177 + IFNG + S100A9 + CX3CR1 + CTLA4 | 0.795 | 0.647 | 0.943 |
| 7 days | CD177 + IFNG + S100A9 + CX3CR1 + TAP2 | 0.776 | 0.623 | 0.928 |
| 7 days | CD177 + IFNG + S100A9 + CX3CR1 + TAP2 + CD3D | 0.778 | 0.628 | 0.929 |
| 7 days | CD177 + IFNG + S100A9 + CX3CR1 + TAP2 + CD3D + CTLA4 | 0.795 | 0.651 | 0.939 |
| 7 days | CD177 + IFNG + S100A9 + CX3CR1 + TAP2 + CTLA4 | 0.795 | 0.651 | 0.939 |
| 7 days | CD177 + IFNG + S100A9 + TAP2 | 0.756 | 0.598 | 0.914 |
| 7 days | CD177 + IFNG + S100A9 + TAP2 + CD3D | 0.753 | 0.594 | 0.913 |
| 7 days | CD177 + IFNG + S100A9 + TAP2 + CD3D + CTLA4 | 0.773 | 0.62 | 0.926 |
| 7 days | CD177 + IFNG + S100A9 + TAP2 + CTLA4 | 0.753 | 0.595 | 0.912 |
| 7 days | CD177 + IFNG + TAP2 | 0.756 | 0.598 | 0.914 |
| 7 days | CD177 + IFNG + TAP2 + CD3D | 0.762 | 0.604 | 0.919 |
| 7 days | CD177 + IFNG + TAP2 + CD3D + CTLA4 | 0.765 | 0.608 | 0.921 |
| 7 days | CD177 + IFNG + TAP2 + CTLA4 | 0.751 | 0.59 | 0.911 |
| 7 days | CD177 + IL1R2 + C3AR1 + CD74 | 0.812 | 0.67 | 0.953 |
| 7 days | CD177 + IL1R2 + C3AR1 + CD74 + CD3D | 0.809 | 0.668 | 0.95 |
| 7 days | CD177 + IL1R2 + C3AR1 + CD74 + CD3D + CTLA4 | 0.806 | 0.664 | 0.948 |
| 7 days | CD177 + IL1R2 + C3AR1 + CD74 + CIITA | 0.875 | 0.763 | 0.988 |
| 7 days | CD177 + IL1R2 + C3AR1 + CD74 + CIITA + CD3D | 0.878 | 0.768 | 0.988 |
| 7 days | CD177 + IL1R2 + C3AR1 + CD74 + CIITA + CD3D + CTLA4 | 0.881 | 0.775 | 0.986 |
| 7 days | CD177 + IL1R2 + C3AR1 + CD74 + CIITA + CTLA4 | 0.873 | 0.761 | 0.985 |
| 7 days | CD177 + IL1R2 + C3AR1 + CD74 + CIITA + TAP2 | 0.875 | 0.763 | 0.988 |
| 7 days | CD177 + IL1R2 + C3AR1 + CD74 + CIITA + TAP2 + CD3D | 0.875 | 0.764 | 0.986 |
| 7 days | CD177 + IL1R2 + C3AR1 + CD74 + CIITA + TAP2 + CD3D + | 0.881 | 0.775 | 0.987 |
| | CTLA4 | | | |
| 7 days | CD177 + IL1R2 + C3AR1 + CD74 + CIITA + TAP2 + CTLA4 | 0.873 | 0.761 | 0.985 |
| 7 days | CD177 + IL1R2 + C3AR1 + CD74 + CTLA4 | 0.817 | 0.678 | 0.957 |
| 7 days | CD177 + IL1R2 + C3AR1 + CD74 + CX3CR1 | 0.828 | 0.688 | 0.969 |
| 7 days | CD177 + IL1R2 + C3AR1 + CD74 + CX3CR1 + CD3D | 0.814 | 0.674 | 0.955 |
| 7 days | CD177 + IL1R2 + C3AR1 + CD74 + CX3CR1 + CD3D + CTLA4 | 0.817 | 0.678 | 0.957 |
| 7 days | CD177 + IL1R2 + C3AR1 + CD74 + CX3CR1 + CIITA | 0.881 | 0.771 | 0.991 |
| 7 days | CD177 + IL1R2 + C3AR1 + CD74 + CX3CR1 + CIITA + CD3D | 0.875 | 0.763 | 0.988 |
| 7 days | CD177 + IL1R2 + C3AR1 + CD74 + CX3CR1 + CIITA + CD3D + | 0.889 | 0.787 | 0.991 |
| | CTLA4 | | | |
| 7 days | CD177 + IL1R2 + C3AR1 + CD74 + CX3CR1 + CIITA + CTLA4 | 0.881 | 0.771 | 0.991 |
| 7 days | CD177 + IL1R2 + C3AR1 + CD74 + CX3CR1 + CIITA + TAP2 | 0.875 | 0.762 | 0.989 |
| 7 days | CD177 + IL1R2 + C3AR1 + CD74 + CX3CR1 + CIITA + TAP2 + | 0.873 | 0.758 | 0.987 |
| | CD3D | | | |
| 7 days | CD177 + IL1R2 + C3AR1 + CD74 + CX3CR1 + CIITA + TAP2 + | 0.898 | 0.801 | 0.994 |
| | CD3D + CTLA4 | | | |
| 7 days | CD177 + IL1R2 + C3AR1 + CD74 + CX3CR1 + CIITA + TAP2 + | 0.884 | 0.775 | 0.992 |
| | CTLA4 | | | |
| 7 days | CD177 + IL1R2 + C3AR1 + CD74 + CX3CR1 + CTLA4 | 0.834 | 0.697 | 0.97 |
| 7 days | CD177 + IL1R2 + C3AR1 + CD74 + CX3CR1 + TAP2 | 0.82 | 0.676 | 0.964 |
| 7 days | CD177 + IL1R2 + C3AR1 + CD74 + CX3CR1 + TAP2 + CD3D | 0.823 | 0.682 | 0.963 |
| 7 days | CD177 + IL1R2 + C3AR1 + CD74 + CX3CR1 + TAP2 + CD3D + | 0.823 | 0.682 | 0.963 |
| | CTLA4 | | | |
| 7 days | CD177 + IL1R2 + C3AR1 + CD74 + CX3CR1 + TAP2 + CTLA4 | 0.831 | 0.692 | 0.97 |
| 7 days | CD177 + IL1R2 + C3AR1 + CD74 + IFNG | 0.842 | 0.719 | 0.965 |
| 7 days | CD177 + IL1R2 + C3AR1 + CD74 + IFNG + CD3D | 0.839 | 0.715 | 0.963 |
| 7 days | CD177 + IL1R2 + C3AR1 + CD74 + IFNG + CD3D + CTLA4 | 0.839 | 0.715 | 0.963 |
| 7 days | CD177 + IL1R2 + C3AR1 + CD74 + IFNG + CIITA | 0.895 | 0.798 | 0.991 |
| 7 days | CD177 + IL1R2 + C3AR1 + CD74 + IFNG + CIITA + CD3D | 0.898 | 0.803 | 0.992 |
| 7 days | CD177 + IL1R2 + C3AR1 + CD74 + IFNG + CIITA + CD3D + | 0.9 | 0.807 | 0.993 |
| | CTLA4 | | | |
| 7 days | CD177 + IL1R2 + C3AR1 + CD74 + IFNG + CIITA + CTLA4 | 0.898 | 0.803 | 0.992 |
| 7 days | CD177 + IL1R2 + C3AR1 + CD74 + IFNG + CIITA + TAP2 | 0.895 | 0.796 | 0.994 |
| 7 days | CD177 + IL1R2 + C3AR1 + CD74 + IFNG + CIITA + TAP2 + | 0.895 | 0.797 | 0.992 |
| | CD3D | | | |
| 7 days | CD177 + IL1R2 + C3AR1 + CD74 + IFNG + CIITA + TAP2 + | 0.9 | 0.806 | 0.994 |
| | CD3D + CTLA4 | | | |
| 7 days | CD177 + IL1R2 + C3AR1 + CD74 + IFNG + CIITA + TAP2 + | 0.889 | 0.788 | 0.991 |
| | CTLA4 | | | |
| 7 days | CD177 + IL1R2 + C3AR1 + CD74 + IFNG + CTLA4 | 0.839 | 0.715 | 0.963 |
| 7 days | CD177 + IL1R2 + C3AR1 + CD74 + IFNG + CX3CR1 | 0.881 | 0.775 | 0.987 |
| 7 days | CD177 + IL1R2 + C3AR1 + CD74 + IFNG + CX3CR1 + CD3D | 0.892 | 0.792 | 0.992 |
| 7 days | CD177 + IL1R2 + C3AR1 + CD74 + IFNG + CX3CR1 + CD3D + | 0.889 | 0.786 | 0.993 |
| | CTLA4 | | | |
| 7 days | CD177 + IL1R2 + C3AR1 + CD74 + IFNG + CX3CR1 + CIITA | 0.917 | 0.827 | 1 |
| 7 days | CD177 + IL1R2 + C3AR1 + CD74 + IFNG + CX3CR1 + CIITA + | 0.928 | 0.84 | 1 |
| | CD3D | | | |
| 7 days | CD177 + IL1R2 + C3AR1 + CD74 + IFNG + CX3CR1 + CIITA + | 0.925 | 0.836 | 1 |
| | CD3D + CTLA4 | | | |
| 7 days | CD177 + IL1R2 + C3AR1 + CD74 + IFNG + CX3CR1 + CIITA + | 0.92 | 0.831 | 1 |
| | CTLA4 | | | |
| 7 days | CD177 + IL1R2 + C3AR1 + CD74 + IFNG + CX3CR1 + CIITA + | 0.917 | 0.827 | 1 |
| | TAP2 | | | |
| 7 days | CD177 + IL1R2 + C3AR1 + CD74 + IFNG + CX3CR1 + CIITA + | 0.934 | 0.848 | 1 |
| | TAP2 + CD3D | | | |
| 7 days | CD177 + IL1R2 + C3AR1 + CD74 + IFNG + CX3CR1 + CIITA + | 0.939 | 0.86 | 1 |
| | TAP2 + CD3D + CTLA4 | | | |
| 7 days | CD177 + IL1R2 + C3AR1 + CD74 + IFNG + CX3CR1 + CIITA + | 0.922 | 0.835 | 1 |
| | TAP2 + CTLA4 | | | |
| 7 days | CD177 + IL1R2 + C3AR1 + CD74 + IFNG + CX3CR1 + CTLA4 | 0.886 | 0.783 | 0.99 |
| 7 days | CD177 + IL1R2 + C3AR1 + CD74 + IFNG + CX3CR1 + TAP2 | 0.881 | 0.775 | 0.987 |
| 7 days | CD177 + IL1R2 + C3AR1 + CD74 + IFNG + CX3CR1 + TAP2 + | 0.892 | 0.79 | 0.994 |
| | CD3D | | | |
| 7 days | CD177 + IL1R2 + C3AR1 + CD74 + IFNG + CX3CR1 + TAP2 + | 0.889 | 0.786 | 0.992 |
| | CD3D + CTLA4 | | | |
| 7 days | CD177 + IL1R2 + C3AR1 + CD74 + IFNG + CX3CR1 + TAP2 + | 0.881 | 0.774 | 0.988 |
| | CTLA4 | | | |
| 7 days | CD177 + IL1R2 + C3AR1 + CD74 + IFNG + S100A9 | 0.839 | 0.715 | 0.963 |
| 7 days | CD177 + IL1R2 + C3AR1 + CD74 + IFNG + S100A9 + CD3D | 0.839 | 0.715 | 0.963 |
| 7 days | CD177 + IL1R2 + C3AR1 + CD74 + IFNG + S100A9 + CD3D + | 0.837 | 0.711 | 0.962 |
| | CTLA4 | | | |
| 7 days | CD177 + IL1R2 + C3AR1 + CD74 + IFNG + S100A9 + CIITA | 0.889 | 0.79 | 0.989 |
| 7 days | CD177 + IL1R2 + C3AR1 + CD74 + IFNG + S100A9 + CIITA + | 0.892 | 0.794 | 0.99 |
| | CD3D | | | |
| 7 days | CD177 + IL1R2 + C3AR1 + CD74 + IFNG + S100A9 + CIITA + | 0.889 | 0.79 | 0.988 |
| | CD3D + CTLA4 | | | |
| 7 days | CD177 + IL1R2 + C3AR1 + CD74 + IFNG + S100A9 + CIITA + | 0.9 | 0.808 | 0.993 |
| | CTLA4 | | | |
| 7 days | CD177 + IL1R2 + C3AR1 + CD74 + IFNG + S100A9 + CIITA + | 0.898 | 0.801 | 0.994 |
| | TAP2 | | | |
| 7 days | CD177 + IL1R2 + C3AR1 + CD74 + IFNG + S100A9 + CIITA + | 0.898 | 0.802 | 0.993 |
| | TAP2 + CD3D | | | |
| 7 days | CD177 + IL1R2 + C3AR1 + CD74 + IFNG + S100A9 + CIITA + | 0.909 | 0.819 | 0.998 |
| | TAP2 + CD3D + CTLA4 | | | |
| 7 days | CD177 + IL1R2 + C3AR1 + CD74 + IFNG + S100A9 + CIITA + | 0.886 | 0.784 | 0.989 |
| | TAP2 + CTLA4 | | | |
| 7 days | CD177 + IL1R2 + C3AR1 + CD74 + IFNG + S100A9 + CTLA4 | 0.837 | 0.712 | 0.961 |
| 7 days | CD177 + IL1R2 + C3AR1 + CD74 + IFNG + S100A9 + CX3CR1 | 0.878 | 0.77 | 0.986 |
| 7 days | CD177 + IL1R2 + C3AR1 + CD74 + IFNG + S100A9 + CX3CR1 + | 0.881 | 0.776 | 0.986 |
| | CD3D | | | |
| 7 days | CD177 + IL1R2 + C3AR1 + CD74 + IFNG + S100A9 + CX3CR1 + | 0.886 | 0.784 | 0.989 |
| | CD3D + CTLA4 | | | |
| 7 days | CD177 + IL1R2 + C3AR1 + CD74 + IFNG + S100A9 + CX3CR1 + | 0.925 | 0.839 | 1 |
| | CIITA | | | |
| 7 days | CD177 + IL1R2 + C3AR1 + CD74 + IFNG + S100A9 + CX3CR1 + | 0.928 | 0.84 | 1 |
| | CIITA + CD3D | | | |
| 7 days | CD177 + IL1R2 + C3AR1 + CD74 + IFNG + S100A9 + CX3CR1 + | 0.925 | 0.84 | 1 |
| | CIITA + CD3D + CTLA4 | | | |
| 7 days | CD177 + IL1R2 + C3AR1 + CD74 + IFNG + S100A9 + CX3CR1 + | 0.925 | 0.839 | 1 |
| | CIITA + CTLA4 | | | |
| 7 days | CD177 + IL1R2 + C3AR1 + CD74 + IFNG + S100A9 + CX3CR1 + | 0.928 | 0.842 | 1 |
| | CIITA + TAP2 | | | |
| 7 days | CD177 + IL1R2 + C3AR1 + CD74 + IFNG + S100A9 + CX3CR1 + | 0.934 | 0.849 | 1 |
| | CIITA + TAP2 + CD3D | | | |
| 7 days | CD177 + IL1R2 + C3AR1 + CD74 + IFNG + S100A9 + CX3CR1 + | 0.936 | 0.855 | 1 |
| | CIITA + TAP2 + CD3D + CTLA4 | | | |
| 7 days | CD177 + IL1R2 + C3AR1 + CD74 + IFNG + S100A9 + CX3CR1 + | 0.928 | 0.844 | 1 |
| | CIITA + TAP2 + CTLA4 | | | |
| 7 days | CD177 + IL1R2 + C3AR1 + CD74 + IFNG + S100A9 + CX3CR1 + | 0.886 | 0.783 | 0.99 |
| | CTLA4 | | | |
| 7 days | CD177 + IL1R2 + C3AR1 + CD74 + IFNG + S100A9 + CX3CR1 + | 0.878 | 0.77 | 0.986 |
| | TAP2 | | | |
| 7 days | CD177 + IL1R2 + C3AR1 + CD74 + IFNG + S100A9 + CX3CR1 + | 0.878 | 0.771 | 0.985 |
| | TAP2 + CD3D | | | |
| 7 days | CD177 + IL1R2 + C3AR1 + CD74 + IFNG + S100A9 + CX3CR1 + | 0.881 | 0.775 | 0.987 |
| | TAP2 + CD3D + CTLA4 | | | |
| 7 days | CD177 + IL1R2 + C3AR1 + CD74 + IFNG + S100A9 + CX3CR1 + | 0.881 | 0.774 | 0.988 |
| | TAP2 + CTLA4 | | | |
| 7 days | CD177 + IL1R2 + C3AR1 + CD74 + IFNG + S100A9 + TAP2 | 0.842 | 0.717 | 0.967 |
| 7 days | CD177 + IL1R2 + C3AR1 + CD74 + IFNG + S100A9 + TAP2 + | 0.842 | 0.718 | 0.966 |
| | CD3D | | | |
| 7 days | CD177 + IL1R2 + C3AR1 + CD74 + IFNG + S100A9 + TAP2 + | 0.842 | 0.718 | 0.966 |
| | CD3D + CTLA4 | | | |
| 7 days | CD177 + IL1R2 + C3AR1 + CD74 + IFNG + S100A9 + TAP2 + | 0.839 | 0.714 | 0.965 |
| | CTLA4 | | | |
| 7 days | CD177 + IL1R2 + C3AR1 + CD74 + IFNG + TAP2 | 0.848 | 0.725 | 0.97 |
| 7 days | CD177 + IL1R2 + C3AR1 + CD74 + IFNG + TAP2 + CD3D | 0.839 | 0.714 | 0.964 |
| 7 days | CD177 + IL1R2 + C3AR1 + CD74 + IFNG + TAP2 + CD3D + | 0.839 | 0.714 | 0.964 |
| | CTLA4 | | | |
| 7 days | CD177 + IL1R2 + C3AR1 + CD74 + IFNG + TAP2 + CTLA4 | 0.837 | 0.709 | 0.964 |
| 7 days | CD177 + IL1R2 + C3AR1 + CD74 + S100A9 | 0.828 | 0.691 | 0.965 |
| 7 days | CD177 + IL1R2 + C3AR1 + CD74 + S100A9 + CD3D | 0.839 | 0.706 | 0.973 |
| 7 days | CD177 + IL1R2 + C3AR1 + CD74 + S100A9 + CD3D + CTLA4 | 0.837 | 0.706 | 0.967 |
| 7 days | CD177 + IL1R2 + C3AR1 + CD74 + S100A9 + CIITA | 0.884 | 0.775 | 0.992 |
| 7 days | CD177 + IL1R2 + C3AR1 + CD74 + S100A9 + CIITA + CD3D | 0.884 | 0.776 | 0.992 |
| 7 days | CD177 + IL1R2 + C3AR1 + CD74 + S100A9 + CIITA + CD3D + | 0.892 | 0.791 | 0.993 |
| | CTLA4 | | | |
| 7 days | CD177 + IL1R2 + C3AR1 + CD74 + S100A9 + CIITA + CTLA4 | 0.875 | 0.766 | 0.985 |
| 7 days | CD177 + IL1R2 + C3AR1 + CD74 + S100A9 + CIITA + TAP2 | 0.881 | 0.771 | 0.99 |
| 7 days | CD177 + IL1R2 + C3AR1 + CD74 + S100A9 + CIITA + TAP2 + | 0.884 | 0.775 | 0.992 |
| | CD3D | | | |
| 7 days | CD177 + IL1R2 + C3AR1 + CD74 + S100A9 + CIITA + TAP2 + | 0.903 | 0.809 | 0.997 |
| | CD3D + CTLA4 | | | |
| 7 days | CD177 + IL1R2 + C3AR1 + CD74 + S100A9 + CIITA + TAP2 + | 0.875 | 0.766 | 0.985 |
| | CTLA4 | | | |
| 7 days | CD177 + IL1R2 + C3AR1 + CD74 + S100A9 + CTLA4 | 0.828 | 0.691 | 0.965 |
| 7 days | CD177 + IL1R2 + C3AR1 + CD74 + S100A9 + CX3CR1 | 0.859 | 0.726 | 0.992 |
| 7 days | CD177 + IL1R2 + C3AR1 + CD74 + S100A9 + CX3CR1 + CD3D | 0.848 | 0.712 | 0.983 |
| 7 days | CD177 + IL1R2 + C3AR1 + CD74 + S100A9 + CX3CR1 + CD3D + | 0.845 | 0.708 | 0.981 |
| | CTLA4 | | | |
| 7 days | CD177 + IL1R2 + C3AR1 + CD74 + S100A9 + CX3CR1 + CIITA | 0.881 | 0.766 | 0.995 |
| 7 days | CD177 + IL1R2 + C3AR1 + CD74 + S100A9 + CX3CR1 + CIITA + | 0.884 | 0.771 | 0.996 |
| | CD3D | | | |
| 7 days | CD177 + IL1R2 + C3AR1 + CD74 + S100A9 + CX3CR1 + CIITA + | 0.9 | 0.802 | 0.999 |
| | CD3D + CTLA4 | | | |
| 7 days | CD177 + IL1R2 + C3AR1 + CD74 + S100A9 + CX3CR1 + CIITA + | 0.878 | 0.767 | 0.989 |
| | CTLA4 | | | |
| 7 days | CD177 + IL1R2 + C3AR1 + CD74 + S100A9 + CX3CR1 + CIITA + | 0.881 | 0.769 | 0.993 |
| | TAP2 | | | |
| 7 days | CD177 + IL1R2 + C3AR1 + CD74 + S100A9 + CX3CR1 + CIITA + | 0.881 | 0.768 | 0.993 |
| | TAP2 + CD3D | | | |
| 7 days | CD177 + IL1R2 + C3AR1 + CD74 + S100A9 + CX3CR1 + CIITA + | 0.909 | 0.814 | 1 |
| | TAP2 + CD3D + CTLA4 | | | |
| 7 days | CD177 + IL1R2 + C3AR1 + CD74 + S100A9 + CX3CR1 + CIITA + | 0.884 | 0.774 | 0.993 |
| | TAP2 + CTLA4 | | | |
| 7 days | CD177 + IL1R2 + C3AR1 + CD74 + S100A9 + CX3CR1 + CTLA4 | 0.861 | 0.731 | 0.992 |
| 7 days | CD177 + IL1R2 + C3AR1 + CD74 + S100A9 + CX3CR1 + TAP2 | 0.859 | 0.725 | 0.992 |
| 7 days | CD177 + IL1R2 + C3AR1 + CD74 + S100A9 + CX3CR1 + TAP2 + | 0.85 | 0.716 | 0.985 |
| | CD3D | | | |
| 7 days | CD177 + IL1R2 + C3AR1 + CD74 + S100A9 + CX3CR1 + TAP2 + | 0.845 | 0.708 | 0.981 |
| | CD3D + CTLA4 | | | |
| 7 days | CD177 + IL1R2 + C3AR1 + CD74 + S100A9 + CX3CR1 + TAP2 + | 0.859 | 0.725 | 0.992 |
| | CTLA4 | | | |
| 7 days | CD177 + IL1R2 + C3AR1 + CD74 + S100A9 + TAP2 | 0.828 | 0.69 | 0.966 |
| 7 days | CD177 + IL1R2 + C3AR1 + CD74 + S100A9 + TAP2 + CD3D | 0.831 | 0.696 | 0.966 |
| 7 days | CD177 + IL1R2 + C3AR1 + CD74 + S100A9 + TAP2 + CD3D + | 0.839 | 0.709 | 0.969 |
| | CTLA4 | | | |
| 7 days | CD177 + IL1R2 + C3AR1 + CD74 + S100A9 + TAP2 + CTLA4 | 0.828 | 0.69 | 0.966 |
| 7 days | CD177 + IL1R2 + C3AR1 + CD74 + TAP2 | 0.817 | 0.677 | 0.958 |
| 7 days | CD177 + IL1R2 + C3AR1 + CD74 + TAP2 + CD3D | 0.814 | 0.674 | 0.955 |
| 7 days | CD177 + IL1R2 + C3AR1 + CD74 + TAP2 + CD3D + CTLA4 | 0.812 | 0.67 | 0.953 |
| 7 days | CD177 + IL1R2 + C3AR1 + CD74 + TAP2 + CTLA4 | 0.814 | 0.672 | 0.956 |
| 7 days | CD177 + IL1R2 + C3AR1 + CIITA | 0.853 | 0.729 | 0.977 |
| 7 days | CD177 + IL1R2 + C3AR1 + CIITA + CD3D | 0.859 | 0.736 | 0.981 |
| 7 days | CD177 + IL1R2 + C3AR1 + CIITA + CD3D + CTLA4 | 0.878 | 0.771 | 0.985 |
| 7 days | CD177 + IL1R2 + C3AR1 + CIITA + CTLA4 | 0.875 | 0.764 | 0.987 |
| 7 days | CD177 + IL1R2 + C3AR1 + CIITA + TAP2 | 0.856 | 0.732 | 0.98 |
| 7 days | CD177 + IL1R2 + C3AR1 + CIITA + TAP2 + CD3D | 0.861 | 0.74 | 0.983 |
| 7 days | CD177 + IL1R2 + C3AR1 + CIITA + TAP2 + CD3D + CTLA4 | 0.875 | 0.765 | 0.986 |
| 7 days | CD177 + IL1R2 + C3AR1 + CIITA + TAP2 + CTLA4 | 0.878 | 0.769 | 0.987 |
| 7 days | CD177 + IL1R2 + C3AR1 + CX3CR1 + CIITA | 0.856 | 0.732 | 0.98 |
| 7 days | CD177 + IL1R2 + C3AR1 + CX3CR1 + CIITA + CD3D | 0.853 | 0.729 | 0.977 |
| 7 days | CD177 + IL1R2 + C3AR1 + CX3CR1 + CIITA + CD3D + CTLA4 | 0.884 | 0.776 | 0.991 |
| 7 days | CD177 + IL1R2 + C3AR1 + CX3CR1 + CIITA + CTLA4 | 0.87 | 0.756 | 0.984 |
| 7 days | CD177 + IL1R2 + C3AR1 + CX3CR1 + CIITA + TAP2 | 0.856 | 0.731 | 0.981 |
| 7 days | CD177 + IL1R2 + C3AR1 + CX3CR1 + CIITA + TAP2 + CD3D | 0.856 | 0.732 | 0.98 |
| 7 days | CD177 + IL1R2 + C3AR1 + CX3CR1 + CIITA + TAP2 + CD3D + | 0.884 | 0.776 | 0.991 |
| | CTLA4 | | | |
| 7 days | CD177 + IL1R2 + C3AR1 + CX3CR1 + CIITA + TAP2 + CTLA4 | 0.873 | 0.759 | 0.986 |
| 7 days | CD177 + IL1R2 + C3AR1 + CX3CR1 + CTLA4 | 0.731 | 0.571 | 0.892 |
| 7 days | CD177 + IL1R2 + C3AR1 + CX3CR1 + TAP2 | 0.767 | 0.612 | 0.922 |
| 7 days | CD177 + IL1R2 + C3AR1 + CX3CR1 + TAP2 + CD3D | 0.767 | 0.614 | 0.921 |
| 7 days | CD177 + IL1R2 + C3AR1 + CX3CR1 + TAP2 + CD3D + CTLA4 | 0.77 | 0.613 | 0.927 |
| 7 days | CD177 + IL1R2 + C3AR1 + CX3CR1 + TAP2 + CTLA4 | 0.767 | 0.609 | 0.926 |
| 7 days | CD177 + IL1R2 + C3AR1 + IFNG | 0.801 | 0.658 | 0.943 |
| 7 days | CD177 + IL1R2 + C3AR1 + IFNG + CD3D | 0.803 | 0.663 | 0.943 |
| 7 days | CD177 + IL1R2 + C3AR1 + IFNG + CD3D + CTLA4 | 0.812 | 0.675 | 0.948 |
| 7 days | CD177 + IL1R2 + C3AR1 + IFNG + CIITA | 0.881 | 0.777 | 0.985 |
| 7 days | CD177 + IL1R2 + C3AR1 + IFNG + CIITA + CD3D | 0.875 | 0.768 | 0.982 |
| 7 days | CD177 + IL1R2 + C3AR1 + IFNG + CIITA + CD3D + CTLA4 | 0.884 | 0.781 | 0.986 |
| 7 days | CD177 + IL1R2 + C3AR1 + IFNG + CIITA + CTLA4 | 0.878 | 0.773 | 0.983 |
| 7 days | CD177 + IL1R2 + C3AR1 + IFNG + CIITA + TAP2 | 0.878 | 0.773 | 0.983 |
| 7 days | CD177 + IL1R2 + C3AR1 + IFNG + CIITA + TAP2 + CD3D | 0.878 | 0.772 | 0.984 |
| 7 days | CD177 + IL1R2 + C3AR1 + IFNG + CIITA + TAP2 + CD3D + | 0.889 | 0.789 | 0.989 |
| | CTLA4 | | | |
| 7 days | CD177 + IL1R2 + C3AR1 + IFNG + CIITA + TAP2 + CTLA4 | 0.878 | 0.773 | 0.983 |
| 7 days | CD177 + IL1R2 + C3AR1 + IFNG + CTLA4 | 0.809 | 0.671 | 0.947 |
| 7 days | CD177 + IL1R2 + C3AR1 + IFNG + CX3CR1 | 0.806 | 0.664 | 0.949 |
| 7 days | CD177 + IL1R2 + C3AR1 + IFNG + CX3CR1 + CD3D | 0.812 | 0.673 | 0.951 |
| 7 days | CD177 + IL1R2 + C3AR1 + IFNG + CX3CR1 + CD3D + CTLA4 | 0.806 | 0.665 | 0.947 |
| 7 days | CD177 + IL1R2 + C3AR1 + IFNG + CX3CR1 + CIITA | 0.875 | 0.768 | 0.982 |
| 7 days | CD177 + IL1R2 + C3AR1 + IFNG + CX3CR1 + CIITA + CD3D | 0.881 | 0.775 | 0.987 |
| 7 days | CD177 + IL1R2 + C3AR1 + IFNG + CX3CR1 + CIITA + CD3D + | 0.903 | 0.808 | 0.999 |
| | CTLA4 | | | |
| 7 days | CD177 + IL1R2 + C3AR1 + IFNG + CX3CR1 + CIITA + CTLA4 | 0.886 | 0.786 | 0.987 |
| 7 days | CD177 + IL1R2 + C3AR1 + IFNG + CX3CR1 + CIITA + TAP2 | 0.878 | 0.772 | 0.984 |
| 7 days | CD177 + IL1R2 + C3AR1 + IFNG + CX3CR1 + CIITA + TAP2 + | 0.892 | 0.793 | 0.991 |
| | CD3D | | | |
| 7 days | CD177 + IL1R2 + C3AR1 + IFNG + CX3CR1 + CIITA + TAP2 + | 0.903 | 0.808 | 0.999 |
| | CD3D + CTLA4 | | | |
| 7 days | CD177 + IL1R2 + C3AR1 + IFNG + CX3CR1 + CIITA + TAP2 + | 0.892 | 0.793 | 0.991 |
| | CTLA4 | | | |
| 7 days | CD177 + IL1R2 + C3AR1 + IFNG + CX3CR1 + CTLA4 | 0.806 | 0.665 | 0.948 |
| 7 days | CD177 + IL1R2 + C3AR1 + IFNG + CX3CR1 + TAP2 | 0.82 | 0.684 | 0.956 |
| 7 days | CD177 + IL1R2 + C3AR1 + IFNG + CX3CR1 + TAP2 + CD3D | 0.817 | 0.68 | 0.954 |
| 7 days | CD177 + IL1R2 + C3AR1 + IFNG + CX3CR1 + TAP2 + CD3D + | 0.82 | 0.684 | 0.956 |
| | CTLA4 | | | |
| 7 days | CD177 + IL1R2 + C3AR1 + IFNG + CX3CR1 + TAP2 + CTLA4 | 0.82 | 0.685 | 0.955 |
| 7 days | CD177 + IL1R2 + C3AR1 + IFNG + S100A9 | 0.795 | 0.651 | 0.939 |
| 7 days | CD177 + IL1R2 + C3AR1 + IFNG + S100A9 + CD3D | 0.795 | 0.651 | 0.939 |
| 7 days | CD177 + IL1R2 + C3AR1 + IFNG + S100A9 + CD3D + CTLA4 | 0.801 | 0.659 | 0.942 |
| 7 days | CD177 + IL1R2 + C3AR1 + IFNG + S100A9 + CIITA | 0.875 | 0.768 | 0.982 |
| 7 days | CD177 + IL1R2 + C3AR1 + IFNG + S100A9 + CIITA + CD3D | 0.875 | 0.768 | 0.982 |
| 7 days | CD177 + IL1R2 + C3AR1 + IFNG + S100A9 + CIITA + CD3D + | 0.875 | 0.769 | 0.982 |
| | CTLA4 | | | |
| 7 days | CD177 + IL1R2 + C3AR1 + IFNG + S100A9 + CIITA + CTLA4 | 0.878 | 0.773 | 0.983 |
| 7 days | CD177 + IL1R2 + C3AR1 + IFNG + S100A9 + CIITA + TAP2 | 0.878 | 0.772 | 0.984 |
| 7 days | CD177 + IL1R2 + C3AR1 + IFNG + S100A9 + CIITA + TAP2 + | 0.878 | 0.772 | 0.984 |
| | CD3D | | | |
| 7 days | CD177 + IL1R2 + C3AR1 + IFNG + S100A9 + CIITA + TAP2 + | 0.884 | 0.782 | 0.985 |
| | CD3D + CTLA4 | | | |
| 7 days | CD177 + IL1R2 + C3AR1 + IFNG + S100A9 + CIITA + TAP2 + | 0.875 | 0.769 | 0.982 |
| | CTLA4 | | | |
| 7 days | CD177 + IL1R2 + C3AR1 + IFNG + S100A9 + CTLA4 | 0.801 | 0.659 | 0.942 |
| 7 days | CD177 + IL1R2 + C3AR1 + IFNG + S100A9 + CX3CR1 | 0.789 | 0.644 | 0.935 |
| 7 days | CD177 + IL1R2 + C3AR1 + IFNG + S100A9 + CX3CR1 + CD3D | 0.798 | 0.657 | 0.939 |
| 7 days | CD177 + IL1R2 + C3AR1 + IFNG + S100A9 + CX3CR1 + CD3D + | 0.792 | 0.65 | 0.935 |
| | CTLA4 | | | |
| 7 days | CD177 + IL1R2 + C3AR1 + IFNG + S100A9 + CX3CR1 + CIITA | 0.881 | 0.775 | 0.986 |
| 7 days | CD177 + IL1R2 + C3AR1 + IFNG + S100A9 + CX3CR1 + CIITA + | 0.884 | 0.78 | 0.988 |
| | CD3D | | | |
| 7 days | CD177 + IL1R2 + C3AR1 + IFNG + S100A9 + CX3CR1 + CIITA + | 0.889 | 0.788 | 0.991 |
| | CD3D + CTLA4 | | | |
| 7 days | CD177 + IL1R2 + C3AR1 + IFNG + S100A9 + CX3CR1 + CIITA + | 0.889 | 0.789 | 0.989 |
| | CTLA4 | | | |
| 7 days | CD177 + IL1R2 + C3AR1 + IFNG + S100A9 + CX3CR1 + CIITA + | 0.898 | 0.798 | 0.997 |
| | TAP2 | | | |
| 7 days | CD177 + IL1R2 + C3AR1 + IFNG + S100A9 + CX3CR1 + CIITA + | 0.892 | 0.793 | 0.991 |
| | TAP2 + CD3D | | | |
| 7 days | CD177 + IL1R2 + C3AR1 + IFNG + S100A9 + CX3CR1 + CIITA + | 0.9 | 0.806 | 0.994 |
| | TAP2 + CD3D + CTLA4 | | | |
| 7 days | CD177 + IL1R2 + C3AR1 + IFNG + S100A9 + CX3CR1 + CIITA + | 0.889 | 0.787 | 0.992 |
| | TAP2 + CTLA4 | | | |
| 7 days | CD177 + IL1R2 + C3AR1 + IFNG + S100A9 + CX3CR1 + CTLA4 | 0.806 | 0.666 | 0.946 |
| 7 days | CD177 + IL1R2 + C3AR1 + IFNG + S100A9 + CX3CR1 + TAP2 | 0.814 | 0.679 | 0.95 |
| 7 days | CD177 + IL1R2 + C3AR1 + IFNG + S100A9 + CX3CR1 + TAP2 + | 0.809 | 0.672 | 0.946 |
| | CD3D | | | |
| 7 days | CD177 + IL1R2 + C3AR1 + IFNG + S100A9 + CX3CR1 + TAP2 + | 0.814 | 0.679 | 0.95 |
| | CD3D + CTLA4 | | | |
| 7 days | CD177 + IL1R2 + C3AR1 + IFNG + S100A9 + CX3CR1 + TAP2 + | 0.814 | 0.679 | 0.95 |
| | CTLA4 | | | |
| 7 days | CD177 + IL1R2 + C3AR1 + IFNG + S100A9 + TAP2 | 0.801 | 0.66 | 0.941 |
| 7 days | CD177 + IL1R2 + C3AR1 + IFNG + S100A9 + TAP2 + CD3D | 0.801 | 0.659 | 0.942 |
| 7 days | CD177 + IL1R2 + C3AR1 + IFNG + S100A9 + TAP2 + CD3D + | 0.803 | 0.662 | 0.945 |
| | CTLA4 | | | |
| 7 days | CD177 + IL1R2 + C3AR1 + IFNG + S100A9 + TAP2 + CTLA4 | 0.798 | 0.656 | 0.94 |
| 7 days | CD177 + IL1R2 + C3AR1 + IFNG + TAP2 | 0.817 | 0.68 | 0.954 |
| 7 days | CD177 + IL1R2 + C3AR1 + IFNG + TAP2 + CD3D | 0.812 | 0.673 | 0.95 |
| 7 days | CD177 + IL1R2 + C3AR1 + IFNG + TAP2 + CD3D + CTLA4 | 0.803 | 0.662 | 0.945 |
| 7 days | CD177 + IL1R2 + C3AR1 + IFNG + TAP2 + CTLA4 | 0.809 | 0.669 | 0.948 |
| 7 days | CD177 + IL1R2 + C3AR1 + S100A9 | 0.767 | 0.615 | 0.92 |
| 7 days | CD177 + IL1R2 + C3AR1 + S100A9 + CD3D | 0.776 | 0.626 | 0.926 |
| 7 days | CD177 + IL1R2 + C3AR1 + S100A9 + CD3D + CTLA4 | 0.767 | 0.616 | 0.918 |
| 7 days | CD177 + IL1R2 + C3AR1 + S100A9 + CIITA | 0.859 | 0.739 | 0.979 |
| 7 days | CD177 + IL1R2 + C3AR1 + S100A9 + CIITA + CD3D | 0.864 | 0.747 | 0.982 |
| 7 days | CD177 + IL1R2 + C3AR1 + S100A9 + CIITA + CD3D + CTLA4 | 0.892 | 0.792 | 0.992 |
| 7 days | CD177 + IL1R2 + C3AR1 + S100A9 + CIITA + CTLA4 | 0.884 | 0.778 | 0.989 |
| 7 days | CD177 + IL1R2 + C3AR1 + S100A9 + CIITA + TAP2 | 0.861 | 0.743 | 0.98 |
| 7 days | CD177 + IL1R2 + C3AR1 + S100A9 + CIITA + TAP2 + CD3D | 0.864 | 0.747 | 0.982 |
| 7 days | CD177 + IL1R2 + C3AR1 + S100A9 + CIITA + TAP2 + CD3D + | 0.889 | 0.789 | 0.989 |
| | CTLA4 | | | |
| 7 days | CD177 + IL1R2 + C3AR1 + S100A9 + CIITA + TAP2 + CTLA4 | 0.881 | 0.773 | 0.988 |
| 7 days | CD177 + IL1R2 + C3AR1 + S100A9 + CTLA4 | 0.767 | 0.614 | 0.92 |
| 7 days | CD177 + IL1R2 + C3AR1 + S100A9 + CX3CR1 | 0.773 | 0.622 | 0.924 |
| 7 days | CD177 + IL1R2 + C3AR1 + S100A9 + CX3CR1 + CD3D | 0.789 | 0.642 | 0.937 |
| 7 days | CD177 + IL1R2 + C3AR1 + S100A9 + CX3CR1 + CD3D + CTLA4 | 0.767 | 0.613 | 0.921 |
| 7 days | CD177 + IL1R2 + C3AR1 + S100A9 + CX3CR1 + CIITA | 0.856 | 0.733 | 0.979 |
| 7 days | CD177 + IL1R2 + C3AR1 + S100A9 + CX3CR1 + CIITA + CD3D | 0.861 | 0.742 | 0.981 |
| 7 days | CD177 + IL1R2 + C3AR1 + S100A9 + CX3CR1 + CIITA + CD3D + | 0.9 | 0.804 | 0.996 |
| | CTLA4 | | | |
| 7 days | CD177 + IL1R2 + C3AR1 + S100A9 + CX3CR1 + CIITA + CTLA4 | 0.873 | 0.761 | 0.984 |
| 7 days | CD177 + IL1R2 + C3AR1 + S100A9 + CX3CR1 + CIITA + TAP2 | 0.861 | 0.74 | 0.983 |
| 7 days | CD177 + IL1R2 + C3AR1 + S100A9 + CX3CR1 + CIITA + TAP2 + | 0.859 | 0.737 | 0.98 |
| | CD3D | | | |
| 7 days | CD177 + IL1R2 + C3AR1 + S100A9 + CX3CR1 + CIITA + TAP2 + | 0.9 | 0.806 | 0.995 |
| | CD3D + CTLA4 | | | |
| 7 days | CD177 + IL1R2 + C3AR1 + S100A9 + CX3CR1 + CIITA + TAP2 + | 0.875 | 0.765 | 0.985 |
| | CTLA4 | | | |
| 7 days | CD177 + IL1R2 + C3AR1 + S100A9 + CX3CR1 + CTLA4 | 0.776 | 0.625 | 0.926 |
| 7 days | CD177 + IL1R2 + C3AR1 + S100A9 + CX3CR1 + TAP2 | 0.778 | 0.626 | 0.93 |
| 7 days | CD177 + IL1R2 + C3AR1 + S100A9 + CX3CR1 + TAP2 + CD3D | 0.781 | 0.631 | 0.932 |
| 7 days | CD177 + IL1R2 + C3AR1 + S100A9 + CX3CR1 + TAP2 + CD3D + | 0.784 | 0.637 | 0.931 |
| | CTLA4 | | | |
| 7 days | CD177 + IL1R2 + C3AR1 + S100A9 + CX3CR1 + TAP2 + CTLA4 | 0.781 | 0.63 | 0.932 |
| 7 days | CD177 + IL1R2 + C3AR1 + S100A9 + TAP2 | 0.795 | 0.649 | 0.941 |
| 7 days | CD177 + IL1R2 + C3AR1 + S100A9 + TAP2 + CD3D | 0.787 | 0.639 | 0.934 |
| 7 days | CD177 + IL1R2 + C3AR1 + S100A9 + TAP2 + CD3D + CTLA4 | 0.787 | 0.64 | 0.933 |
| 7 days | CD177 + IL1R2 + C3AR1 + S100A9 + TAP2 + CTLA4 | 0.795 | 0.649 | 0.941 |
| 7 days | CD177 + IL1R2 + C3AR1 + TAP2 | 0.767 | 0.612 | 0.922 |
| 7 days | CD177 + IL1R2 + C3AR1 + TAP2 + CD3D | 0.765 | 0.609 | 0.92 |
| 7 days | CD177 + IL1R2 + C3AR1 + TAP2 + CD3D + CTLA4 | 0.773 | 0.62 | 0.926 |
| 7 days | CD177 + IL1R2 + C3AR1 + TAP2 + CTLA4 | 0.765 | 0.61 | 0.919 |
| 7 days | CD177 + IL1R2 + CD74 | 0.773 | 0.619 | 0.927 |
| 7 days | CD177 + IL1R2 + CD74 + CD3D | 0.759 | 0.603 | 0.915 |
| 7 days | CD177 + IL1R2 + CD74 + CD3D + CTLA4 | 0.767 | 0.613 | 0.921 |
| 7 days | CD177 + IL1R2 + CD74 + CIITA | 0.812 | 0.668 | 0.955 |
| 7 days | CD177 + IL1R2 + CD74 + CIITA + CD3D | 0.814 | 0.672 | 0.957 |
| 7 days | CD177 + IL1R2 + CD74 + CIITA + CD3D + CTLA4 | 0.828 | 0.688 | 0.968 |
| 7 days | CD177 + IL1R2 + CD74 + CIITA + CTLA4 | 0.814 | 0.67 | 0.959 |
| 7 days | CD177 + IL1R2 + CD74 + CIITA + TAP2 | 0.809 | 0.664 | 0.954 |
| 7 days | CD177 + IL1R2 + CD74 + CIITA + TAP2 + CD3D | 0.817 | 0.675 | 0.96 |
| 7 days | CD177 + IL1R2 + CD74 + CIITA + TAP2 + CD3D + CTLA4 | 0.825 | 0.685 | 0.966 |
| 7 days | CD177 + IL1R2 + CD74 + CIITA + TAP2 + CTLA4 | 0.82 | 0.678 | 0.961 |
| 7 days | CD177 + IL1R2 + CD74 + CTLA4 | 0.765 | 0.608 | 0.921 |
| 7 days | CD177 + IL1R2 + CD74 + CX3CR1 | 0.778 | 0.619 | 0.938 |
| 7 days | CD177 + IL1R2 + CD74 + CX3CR1 + CD3D | 0.773 | 0.619 | 0.927 |
| 7 days | CD177 + IL1R2 + CD74 + CX3CR1 + CD3D + CTLA4 | 0.759 | 0.598 | 0.92 |
| 7 days | CD177 + IL1R2 + CD74 + CX3CR1 + CIITA | 0.82 | 0.675 | 0.965 |
| 7 days | CD177 + IL1R2 + CD74 + CX3CR1 + CIITA + CD3D | 0.803 | 0.654 | 0.953 |
| 7 days | CD177 + IL1R2 + CD74 + CX3CR1 + CIITA + CD3D + CTLA4 | 0.823 | 0.682 | 0.963 |
| 7 days | CD177 + IL1R2 + CD74 + CX3CR1 + CIITA + CTLA4 | 0.82 | 0.675 | 0.965 |
| 7 days | CD177 + IL1R2 + CD74 + CX3CR1 + CIITA + TAP2 | 0.825 | 0.681 | 0.97 |
| 7 days | CD177 + IL1R2 + CD74 + CX3CR1 + CIITA + TAP2 + CD3D | 0.82 | 0.679 | 0.961 |
| 7 days | CD177 + IL1R2 + CD74 + CX3CR1 + CIITA + TAP2 + CD3D + | 0.842 | 0.711 | 0.973 |
| | CTLA4 | | | |
| 7 days | CD177 + IL1R2 + CD74 + CX3CR1 + CIITA + TAP2 + CTLA4 | 0.823 | 0.679 | 0.967 |
| 7 days | CD177 + IL1R2 + CD74 + CX3CR1 + CTLA4 | 0.776 | 0.616 | 0.935 |
| 7 days | CD177 + IL1R2 + CD74 + CX3CR1 + TAP2 | 0.787 | 0.631 | 0.943 |
| 7 days | CD177 + IL1R2 + CD74 + CX3CR1 + TAP2 + CD3D | 0.77 | 0.615 | 0.925 |
| 7 days | CD177 + IL1R2 + CD74 + CX3CR1 + TAP2 + CD3D + CTLA4 | 0.778 | 0.623 | 0.934 |
| 7 days | CD177 + IL1R2 + CD74 + CX3CR1 + TAP2 + CTLA4 | 0.781 | 0.625 | 0.937 |
| 7 days | CD177 + IL1R2 + CD74 + IFNG | 0.778 | 0.631 | 0.926 |
| 7 days | CD177 + IL1R2 + CD74 + IFNG + CD3D | 0.776 | 0.627 | 0.924 |
| 7 days | CD177 + IL1R2 + CD74 + IFNG + CD3D + CTLA4 | 0.795 | 0.652 | 0.938 |
| 7 days | CD177 + IL1R2 + CD74 + IFNG + CIITA | 0.814 | 0.678 | 0.951 |
| 7 days | CD177 + IL1R2 + CD74 + IFNG + CIITA + CD3D | 0.814 | 0.678 | 0.951 |
| 7 days | CD177 + IL1R2 + CD74 + IFNG + CIITA + CD3D + CTLA4 | 0.814 | 0.679 | 0.95 |
| 7 days | CD177 + IL1R2 + CD74 + IFNG + CIITA + CTLA4 | 0.82 | 0.686 | 0.954 |
| 7 days | CD177 + IL1R2 + CD74 + IFNG + CIITA + TAP2 | 0.828 | 0.7 | 0.957 |
| 7 days | CD177 + IL1R2 + CD74 + IFNG + CIITA + TAP2 + CD3D | 0.828 | 0.7 | 0.956 |
| 7 days | CD177 + IL1R2 + CD74 + IFNG + CIITA + TAP2 + CD3D + | 0.831 | 0.703 | 0.959 |
| | CTLA4 | | | |
| 7 days | CD177 + IL1R2 + CD74 + IFNG + CIITA + TAP2 + CTLA4 | 0.823 | 0.692 | 0.954 |
| 7 days | CD177 + IL1R2 + CD74 + IFNG + CTLA4 | 0.776 | 0.628 | 0.924 |
| 7 days | CD177 + IL1R2 + CD74 + IFNG + CX3CR1 | 0.831 | 0.7 | 0.962 |
| 7 days | CD177 + IL1R2 + CD74 + IFNG + CX3CR1 + CD3D | 0.845 | 0.718 | 0.972 |
| 7 days | CD177 + IL1R2 + CD74 + IFNG + CX3CR1 + CD3D + CTLA4 | 0.85 | 0.728 | 0.973 |
| 7 days | CD177 + IL1R2 + CD74 + IFNG + CX3CR1 + CIITA | 0.889 | 0.784 | 0.994 |
| 7 days | CD177 + IL1R2 + CD74 + IFNG + CX3CR1 + CIITA + CD3D | 0.9 | 0.802 | 0.999 |
| 7 days | CD177 + IL1R2 + CD74 + IFNG + CX3CR1 + CIITA + CD3D + | 0.895 | 0.794 | 0.995 |
| | CTLA4 | | | |
| 7 days | CD177 + IL1R2 + CD74 + IFNG + CX3CR1 + CIITA + CTLA4 | 0.889 | 0.782 | 0.996 |
| 7 days | CD177 + IL1R2 + CD74 + IFNG + CX3CR1 + CIITA + TAP2 | 0.886 | 0.78 | 0.993 |
| 7 days | CD177 + IL1R2 + CD74 + IFNG + CX3CR1 + CIITA + TAP2 + | 0.906 | 0.81 | 1 |
| | CD3D | | | |
| 7 days | CD177 + IL1R2 + CD74 + IFNG + CX3CR1 + CIITA + TAP2 + | 0.906 | 0.81 | 1 |
| | CD3D + CTLA4 | | | |
| 7 days | CD177 + IL1R2 + CD74 + IFNG + CX3CR1 + CIITA + TAP2 + | 0.889 | 0.782 | 0.996 |
| | CTLA4 | | | |
| 7 days | CD177 + IL1R2 + CD74 + IFNG + CX3CR1 + CTLA4 | 0.853 | 0.732 | 0.974 |
| 7 days | CD177 + IL1R2 + CD74 + IFNG + CX3CR1 + TAP2 | 0.831 | 0.701 | 0.961 |
| 7 days | CD177 + IL1R2 + CD74 + IFNG + CX3CR1 + TAP2 + CD3D | 0.845 | 0.718 | 0.972 |
| 7 days | CD177 + IL1R2 + CD74 + IFNG + CX3CR1 + TAP2 + CD3D + | 0.848 | 0.723 | 0.972 |
| | CTLA4 | | | |
| 7 days | CD177 + IL1R2 + CD74 + IFNG + CX3CR1 + TAP2 + CTLA4 | 0.853 | 0.732 | 0.975 |
| 7 days | CD177 + IL1R2 + CD74 + IFNG + S100A9 | 0.776 | 0.627 | 0.925 |
| 7 days | CD177 + IL1R2 + CD74 + IFNG + S100A9 + CD3D | 0.77 | 0.62 | 0.92 |
| 7 days | CD177 + IL1R2 + CD74 + IFNG + S100A9 + CD3D + CTLA4 | 0.795 | 0.652 | 0.938 |
| 7 days | CD177 + IL1R2 + CD74 + IFNG + S100A9 + CIITA | 0.814 | 0.678 | 0.951 |
| 7 days | CD177 + IL1R2 + CD74 + IFNG + S100A9 + CIITA + CD3D | 0.814 | 0.678 | 0.951 |
| 7 days | CD177 + IL1R2 + CD74 + IFNG + S100A9 + CIITA + CD3D + | 0.817 | 0.683 | 0.951 |
| | CTLA4 | | | |
| 7 days | CD177 + IL1R2 + CD74 + IFNG + S100A9 + CIITA + CTLA4 | 0.817 | 0.682 | 0.952 |
| 7 days | CD177 + IL1R2 + CD74 + IFNG + S100A9 + CIITA + TAP2 | 0.831 | 0.704 | 0.958 |
| 7 days | CD177 + IL1R2 + CD74 + IFNG + S100A9 + CIITA + TAP2 + | 0.837 | 0.712 | 0.961 |
| | CD3D | | | |
| 7 days | CD177 + IL1R2 + CD74 + IFNG + S100A9 + CIITA + TAP2 + | 0.831 | 0.704 | 0.958 |
| | CD3D + CTLA4 | | | |
| 7 days | CD177 + IL1R2 + CD74 + IFNG + S100A9 + CIITA + TAP2 + | 0.823 | 0.692 | 0.954 |
| | CTLA4 | | | |
| 7 days | CD177 + IL1R2 + CD74 + IFNG + S100A9 + CTLA4 | 0.778 | 0.631 | 0.925 |
| 7 days | CD177 + IL1R2 + CD74 + IFNG + S100A9 + CX3CR1 | 0.834 | 0.705 | 0.963 |
| 7 days | CD177 + IL1R2 + CD74 + IFNG + S100A9 + CX3CR1 + CD3D | 0.842 | 0.715 | 0.969 |
| 7 days | CD177 + IL1R2 + CD74 + IFNG + S100A9 + CX3CR1 + CD3D + | 0.848 | 0.722 | 0.973 |
| | CTLA4 | | | |
| 7 days | CD177 + IL1R2 + CD74 + IFNG + S100A9 + CX3CR1 + CIITA | 0.895 | 0.792 | 0.998 |
| 7 days | CD177 + IL1R2 + CD74 + IFNG + S100A9 + CX3CR1 + CIITA + | 0.909 | 0.815 | 1 |
| | CD3D | | | |
| 7 days | CD177 + IL1R2 + CD74 + IFNG + S100A9 + CX3CR1 + CIITA + | 0.898 | 0.798 | 0.997 |
| | CD3D + CTLA4 | | | |
| 7 days | CD177 + IL1R2 + CD74 + IFNG + S100A9 + CX3CR1 + CIITA + | 0.889 | 0.782 | 0.996 |
| | CTLA4 | | | |
| 7 days | CD177 + IL1R2 + CD74 + IFNG + S100A9 + CX3CR1 + CIITA + | 0.895 | 0.792 | 0.998 |
| | TAP2 | | | |
| 7 days | CD177 + IL1R2 + CD74 + IFNG + S100A9 + CX3CR1 + CIITA + | 0.911 | 0.818 | 1 |
| | TAP2 + CD3D | | | |
| 7 days | CD177 + IL1R2 + CD74 + IFNG + S100A9 + CX3CR1 + CIITA + | 0.903 | 0.807 | 0.999 |
| | TAP2 + CD3D + CTLA4 | | | |
| 7 days | CD177 + IL1R2 + CD74 + IFNG + S100A9 + CX3CR1 + CIITA + | 0.889 | 0.782 | 0.996 |
| | TAP2 + CTLA4 | | | |
| 7 days | CD177 + IL1R2 + CD74 + IFNG + S100A9 + CX3CR1 + CTLA4 | 0.864 | 0.749 | 0.98 |
| 7 days | CD177 + IL1R2 + CD74 + IFNG + S100A9 + CX3CR1 + TAP2 | 0.831 | 0.7 | 0.962 |
| 7 days | CD177 + IL1R2 + CD74 + IFNG + S100A9 + CX3CR1 + TAP2 + | 0.848 | 0.723 | 0.973 |
| | CD3D | | | |
| 7 days | CD177 + IL1R2 + CD74 + IFNG + S100A9 + CX3CR1 + TAP2 + | 0.842 | 0.715 | 0.969 |
| | CD3D + CTLA4 | | | |
| 7 days | CD177 + IL1R2 + CD74 + IFNG + S100A9 + CX3CR1 + TAP2 + | 0.867 | 0.753 | 0.981 |
| | CTLA4 | | | |
| 7 days | CD177 + IL1R2 + CD74 + IFNG + S100A9 + TAP2 | 0.801 | 0.659 | 0.942 |
| 7 days | CD177 + IL1R2 + CD74 + IFNG + S100A9 + TAP2 + CD3D | 0.787 | 0.643 | 0.931 |
| 7 days | CD177 + IL1R2 + CD74 + IFNG + S100A9 + TAP2 + CD3D + | 0.781 | 0.635 | 0.928 |
| | CTLA4 | | | |
| 7 days | CD177 + IL1R2 + CD74 + IFNG + S100A9 + TAP2 + CTLA4 | 0.789 | 0.644 | 0.935 |
| 7 days | CD177 + IL1R2 + CD74 + IFNG + TAP2 | 0.792 | 0.647 | 0.938 |
| 7 days | CD177 + IL1R2 + CD74 + IFNG + TAP2 + CD3D | 0.789 | 0.645 | 0.934 |
| 7 days | CD177 + IL1R2 + CD74 + IFNG + TAP2 + CD3D + CTLA4 | 0.787 | 0.642 | 0.932 |
| 7 days | CD177 + IL1R2 + CD74 + IFNG + TAP2 + CTLA4 | 0.784 | 0.638 | 0.93 |
| 7 days | CD177 + IL1R2 + CD74 + S100A9 | 0.776 | 0.622 | 0.93 |
| 7 days | CD177 + IL1R2 + CD74 + S100A9 + CD3D | 0.784 | 0.634 | 0.934 |
| 7 days | CD177 + IL1R2 + CD74 + S100A9 + CD3D + CTLA4 | 0.798 | 0.654 | 0.942 |
| 7 days | CD177 + IL1R2 + CD74 + S100A9 + CIITA | 0.817 | 0.677 | 0.957 |
| 7 days | CD177 + IL1R2 + CD74 + S100A9 + CIITA + CD3D | 0.825 | 0.689 | 0.962 |
| 7 days | CD177 + IL1R2 + CD74 + S100A9 + CIITA + CD3D + CTLA4 | 0.825 | 0.689 | 0.962 |
| 7 days | CD177 + IL1R2 + CD74 + S100A9 + CIITA + CTLA4 | 0.812 | 0.668 | 0.956 |
| 7 days | CD177 + IL1R2 + CD74 + S100A9 + CIITA + TAP2 | 0.82 | 0.681 | 0.959 |
| 7 days | CD177 + IL1R2 + CD74 + S100A9 + CIITA + TAP2 + CD3D | 0.828 | 0.692 | 0.964 |
| 7 days | CD177 + IL1R2 + CD74 + S100A9 + CIITA + TAP2 + CD3D + | 0.839 | 0.71 | 0.968 |
| | CTLA4 | | | |
| 7 days | CD177 + IL1R2 + CD74 + S100A9 + CIITA + TAP2 + CTLA4 | 0.812 | 0.668 | 0.956 |
| 7 days | CD177 + IL1R2 + CD74 + S100A9 + CTLA4 | 0.778 | 0.626 | 0.931 |
| 7 days | CD177 + IL1R2 + CD74 + S100A9 + CX3CR1 | 0.825 | 0.676 | 0.975 |
| 7 days | CD177 + IL1R2 + CD74 + S100A9 + CX3CR1 + CD3D | 0.806 | 0.66 | 0.952 |
| 7 days | CD177 + IL1R2 + CD74 + S100A9 + CX3CR1 + CD3D + CTLA4 | 0.801 | 0.654 | 0.947 |
| 7 days | CD177 + IL1R2 + CD74 + S100A9 + CX3CR1 + CIITA | 0.837 | 0.697 | 0.977 |
| 7 days | CD177 + IL1R2 + CD74 + S100A9 + CX3CR1 + CIITA + CD3D | 0.845 | 0.714 | 0.976 |
| 7 days | CD177 + IL1R2 + CD74 + S100A9 + CX3CR1 + CIITA + CD3D + | 0.848 | 0.724 | 0.971 |
| | CTLA4 | | | |
| 7 days | CD177 + IL1R2 + CD74 + S100A9 + CX3CR1 + CIITA + CTLA4 | 0.837 | 0.697 | 0.977 |
| 7 days | CD177 + IL1R2 + CD74 + S100A9 + CX3CR1 + CIITA + TAP2 | 0.834 | 0.694 | 0.974 |
| 7 days | CD177 + IL1R2 + CD74 + S100A9 + CX3CR1 + CIITA + TAP2 + | 0.839 | 0.707 | 0.971 |
| | CD3D | | | |
| 7 days | CD177 + IL1R2 + CD74 + S100A9 + CX3CR1 + CIITA + TAP2 + | 0.848 | 0.725 | 0.97 |
| | CD3D + CTLA4 | | | |
| 7 days | CD177 + IL1R2 + CD74 + S100A9 + CX3CR1 + CIITA + TAP2 + | 0.828 | 0.687 | 0.97 |
| | CTLA4 | | | |
| 7 days | CD177 + IL1R2 + CD74 + S100A9 + CX3CR1 + CTLA4 | 0.82 | 0.67 | 0.97 |
| 7 days | CD177 + IL1R2 + CD74 + S100A9 + CX3CR1 + TAP2 | 0.817 | 0.664 | 0.97 |
| 7 days | CD177 + IL1R2 + CD74 + S100A9 + CX3CR1 + TAP2 + CD3D | 0.809 | 0.664 | 0.954 |
| 7 days | CD177 + IL1R2 + CD74 + S100A9 + CX3CR1 + TAP2 + CD3D + | 0.801 | 0.654 | 0.947 |
| | CTLA4 | | | |
| 7 days | CD177 + IL1R2 + CD74 + S100A9 + CX3CR1 + TAP2 + CTLA4 | 0.814 | 0.665 | 0.964 |
| 7 days | CD177 + IL1R2 + CD74 + S100A9 + TAP2 | 0.77 | 0.615 | 0.925 |
| 7 days | CD177 + IL1R2 + CD74 + S100A9 + TAP2 + CD3D | 0.789 | 0.642 | 0.937 |
| 7 days | CD177 + IL1R2 + CD74 + S100A9 + TAP2 + CD3D + CTLA4 | 0.795 | 0.648 | 0.942 |
| 7 days | CD177 + IL1R2 + CD74 + S100A9 + TAP2 + CTLA4 | 0.778 | 0.626 | 0.931 |
| 7 days | CD177 + IL1R2 + CD74 + TAP2 | 0.776 | 0.625 | 0.926 |
| 7 days | CD177 + IL1R2 + CD74 + TAP2 + CD3D | 0.77 | 0.616 | 0.924 |
| 7 days | CD177 + IL1R2 + CD74 + TAP2 + CD3D + CTLA4 | 0.776 | 0.623 | 0.928 |
| 7 days | CD177 + IL1R2 + CD74 + TAP2 + CTLA4 | 0.77 | 0.615 | 0.925 |
| 7 days | CD177 + IL1R2 + CIITA | 0.814 | 0.671 | 0.958 |
| 7 days | CD177 + IL1R2 + CIITA + CD3D | 0.814 | 0.669 | 0.96 |
| 7 days | CD177 + IL1R2 + CIITA + CD3D + CTLA4 | 0.834 | 0.695 | 0.973 |
| 7 days | CD177 + IL1R2 + CIITA + CTLA4 | 0.814 | 0.671 | 0.958 |
| 7 days | CD177 + IL1R2 + CIITA + TAP2 | 0.814 | 0.671 | 0.957 |
| 7 days | CD177 + IL1R2 + CIITA + TAP2 + CD3D | 0.817 | 0.674 | 0.96 |
| 7 days | CD177 + IL1R2 + CIITA + TAP2 + CD3D + CTLA4 | 0.834 | 0.695 | 0.973 |
| 7 days | CD177 + IL1R2 + CIITA + TAP2 + CTLA4 | 0.831 | 0.694 | 0.968 |
| 7 days | CD177 + IL1R2 + CX3CR1 + CD3D + CTLA4 | 0.731 | 0.566 | 0.897 |
| 7 days | CD177 + IL1R2 + CX3CR1 + CIITA | 0.817 | 0.674 | 0.96 |
| 7 days | CD177 + IL1R2 + CX3CR1 + CIITA + CD3D | 0.817 | 0.674 | 0.96 |
| 7 days | CD177 + IL1R2 + CX3CR1 + CIITA + CD3D + CTLA4 | 0.831 | 0.693 | 0.97 |
| 7 days | CD177 + IL1R2 + CX3CR1 + CIITA + CTLA4 | 0.82 | 0.677 | 0.963 |
| 7 days | CD177 + IL1R2 + CX3CR1 + CIITA + TAP2 | 0.823 | 0.682 | 0.963 |
| 7 days | CD177 + IL1R2 + CX3CR1 + CIITA + TAP2 + CD3D | 0.814 | 0.672 | 0.956 |
| 7 days | CD177 + IL1R2 + CX3CR1 + CIITA + TAP2 + CD3D + CTLA4 | 0.837 | 0.703 | 0.97 |
| 7 days | CD177 + IL1R2 + CX3CR1 + CIITA + TAP2 + CTLA4 | 0.823 | 0.68 | 0.966 |
| 7 days | CD177 + IL1R2 + CX3CR1 + TAP2 + CD3D + CTLA4 | 0.731 | 0.563 | 0.9 |
| 7 days | CD177 + IL1R2 + CX3CR1 + TAP2 + CTLA4 | 0.737 | 0.571 | 0.903 |
| 7 days | CD177 + IL1R2 + IFNG | 0.762 | 0.607 | 0.916 |
| 7 days | CD177 + IL1R2 + IFNG + CD3D | 0.756 | 0.601 | 0.912 |
| 7 days | CD177 + IL1R2 + IFNG + CD3D + CTLA4 | 0.748 | 0.592 | 0.904 |
| 7 days | CD177 + IL1R2 + IFNG + CIITA | 0.814 | 0.679 | 0.949 |
| 7 days | CD177 + IL1R2 + IFNG + CIITA + CD3D | 0.814 | 0.679 | 0.949 |
| 7 days | CD177 + IL1R2 + IFNG + CIITA + CD3D + CTLA4 | 0.817 | 0.682 | 0.953 |
| 7 days | CD177 + IL1R2 + IFNG + CIITA + CTLA4 | 0.812 | 0.675 | 0.948 |
| 7 days | CD177 + IL1R2 + IFNG + CIITA + TAP2 | 0.817 | 0.683 | 0.951 |
| 7 days | CD177 + IL1R2 + IFNG + CIITA + TAP2 + CD3D | 0.812 | 0.675 | 0.948 |
| 7 days | CD177 + IL1R2 + IFNG + CIITA + TAP2 + CD3D + CTLA4 | 0.823 | 0.69 | 0.956 |
| 7 days | CD177 + IL1R2 + IFNG + CIITA + TAP2 + CTLA4 | 0.812 | 0.675 | 0.948 |
| 7 days | CD177 + IL1R2 + IFNG + CTLA4 | 0.748 | 0.591 | 0.905 |
| 7 days | CD177 + IL1R2 + IFNG + CX3CR1 | 0.781 | 0.625 | 0.938 |
| 7 days | CD177 + IL1R2 + IFNG + CX3CR1 + CD3D | 0.781 | 0.624 | 0.938 |
| 7 days | CD177 + IL1R2 + IFNG + CX3CR1 + CD3D + CTLA4 | 0.792 | 0.641 | 0.944 |
| 7 days | CD177 + IL1R2 + IFNG + CX3CR1 + CIITA | 0.861 | 0.741 | 0.982 |
| 7 days | CD177 + IL1R2 + IFNG + CX3CR1 + CIITA + CD3D | 0.856 | 0.736 | 0.976 |
| 7 days | CD177 + IL1R2 + IFNG + CX3CR1 + CIITA + CD3D + CTLA4 | 0.859 | 0.739 | 0.978 |
| 7 days | CD177 + IL1R2 + IFNG + CX3CR1 + CIITA + CTLA4 | 0.861 | 0.74 | 0.983 |
| 7 days | CD177 + IL1R2 + IFNG + CX3CR1 + CIITA + TAP2 | 0.859 | 0.743 | 0.975 |
| 7 days | CD177 + IL1R2 + IFNG + CX3CR1 + CIITA + TAP2 + CD3D | 0.85 | 0.731 | 0.97 |
| 7 days | CD177 + IL1R2 + IFNG + CX3CR1 + CIITA + TAP2 + CD3D + | 0.859 | 0.743 | 0.974 |
| | CTLA4 | | | |
| 7 days | CD177 + IL1R2 + IFNG + CX3CR1 + CIITA + TAP2 + CTLA4 | 0.867 | 0.754 | 0.98 |
| 7 days | CD177 + IL1R2 + IFNG + CX3CR1 + CTLA4 | 0.792 | 0.641 | 0.944 |
| 7 days | CD177 + IL1R2 + IFNG + CX3CR1 + TAP2 | 0.784 | 0.632 | 0.935 |
| 7 days | CD177 + IL1R2 + IFNG + CX3CR1 + TAP2 + CD3D | 0.781 | 0.629 | 0.934 |
| 7 days | CD177 + IL1R2 + IFNG + CX3CR1 + TAP2 + CD3D + CTLA4 | 0.801 | 0.658 | 0.943 |
| 7 days | CD177 + IL1R2 + IFNG + CX3CR1 + TAP2 + CTLA4 | 0.795 | 0.651 | 0.939 |
| 7 days | CD177 + IL1R2 + IFNG + S100A9 | 0.756 | 0.602 | 0.911 |
| 7 days | CD177 + IL1R2 + IFNG + S100A9 + CD3D | 0.762 | 0.61 | 0.914 |
| 7 days | CD177 + IL1R2 + IFNG + S100A9 + CD3D + CTLA4 | 0.756 | 0.602 | 0.91 |
| 7 days | CD177 + IL1R2 + IFNG + S100A9 + CIITA | 0.812 | 0.676 | 0.947 |
| 7 days | CD177 + IL1R2 + IFNG + S100A9 + CIITA + CD3D | 0.814 | 0.681 | 0.948 |
| 7 days | CD177 + IL1R2 + IFNG + S100A9 + CIITA + CD3D + CTLA4 | 0.82 | 0.688 | 0.952 |
| 7 days | CD177 + IL1R2 + IFNG + S100A9 + CIITA + CTLA4 | 0.814 | 0.679 | 0.95 |
| 7 days | CD177 + IL1R2 + IFNG + S100A9 + CIITA + TAP2 | 0.814 | 0.679 | 0.949 |
| 7 days | CD177 + IL1R2 + IFNG + S100A9 + CIITA + TAP2 + CD3D | 0.823 | 0.692 | 0.954 |
| 7 days | CD177 + IL1R2 + IFNG + S100A9 + CIITA + TAP2 + CD3D + | 0.823 | 0.691 | 0.955 |
| | CTLA4 | | | |
| 7 days | CD177 + IL1R2 + IFNG + S100A9 + CIITA + TAP2 + CTLA4 | 0.817 | 0.683 | 0.951 |
| 7 days | CD177 + IL1R2 + IFNG + S100A9 + CTLA4 | 0.756 | 0.602 | 0.91 |
| 7 days | CD177 + IL1R2 + IFNG + S100A9 + CX3CR1 | 0.784 | 0.635 | 0.933 |
| 7 days | CD177 + IL1R2 + IFNG + S100A9 + CX3CR1 + CD3D | 0.795 | 0.652 | 0.938 |
| 7 days | CD177 + IL1R2 + IFNG + S100A9 + CX3CR1 + CD3D + CTLA4 | 0.803 | 0.661 | 0.945 |
| 7 days | CD177 + IL1R2 + IFNG + S100A9 + CX3CR1 + CIITA | 0.861 | 0.741 | 0.982 |
| 7 days | CD177 + IL1R2 + IFNG + S100A9 + CX3CR1 + CIITA + CD3D | 0.861 | 0.745 | 0.978 |
| 7 days | CD177 + IL1R2 + IFNG + S100A9 + CX3CR1 + CIITA + CD3D + | 0.867 | 0.754 | 0.98 |
| | CTLA4 | | | |
| 7 days | CD177 + IL1R2 + IFNG + S100A9 + CX3CR1 + CIITA + CTLA4 | 0.861 | 0.74 | 0.983 |
| 7 days | CD177 + IL1R2 + IFNG + S100A9 + CX3CR1 + CIITA + TAP2 | 0.861 | 0.747 | 0.976 |
| 7 days | CD177 + IL1R2 + IFNG + S100A9 + CX3CR1 + CIITA + TAP2 + | 0.853 | 0.735 | 0.972 |
| | CD3D | | | |
| 7 days | CD177 + IL1R2 + IFNG + S100A9 + CX3CR1 + CIITA + TAP2 + | 0.87 | 0.759 | 0.98 |
| | CD3D + CTLA4 | | | |
| 7 days | CD177 + IL1R2 + IFNG + S100A9 + CX3CR1 + CIITA + TAP2 + | 0.861 | 0.746 | 0.977 |
| | CTLA4 | | | |
| 7 days | CD177 + IL1R2 + IFNG + S100A9 + CX3CR1 + CTLA4 | 0.803 | 0.66 | 0.947 |
| 7 days | CD177 + IL1R2 + IFNG + S100A9 + CX3CR1 + TAP2 | 0.77 | 0.617 | 0.924 |
| 7 days | CD177 + IL1R2 + IFNG + S100A9 + CX3CR1 + TAP2 + CD3D | 0.784 | 0.637 | 0.93 |
| 7 days | CD177 + IL1R2 + IFNG + S100A9 + CX3CR1 + TAP2 + CD3D + | 0.795 | 0.652 | 0.938 |
| | CTLA4 | | | |
| 7 days | CD177 + IL1R2 + IFNG + S100A9 + CX3CR1 + TAP2 + CTLA4 | 0.795 | 0.652 | 0.938 |
| 7 days | CD177 + IL1R2 + IFNG + S100A9 + TAP2 | 0.759 | 0.606 | 0.912 |
| 7 days | CD177 + IL1R2 + IFNG + S100A9 + TAP2 + CD3D | 0.759 | 0.606 | 0.912 |
| 7 days | CD177 + IL1R2 + IFNG + S100A9 + TAP2 + CD3D + CTLA4 | 0.753 | 0.598 | 0.908 |
| 7 days | CD177 + IL1R2 + IFNG + S100A9 + TAP2 + CTLA4 | 0.762 | 0.609 | 0.914 |
| 7 days | CD177 + IL1R2 + IFNG + TAP2 | 0.759 | 0.605 | 0.913 |
| 7 days | CD177 + IL1R2 + IFNG + TAP2 + CD3D | 0.756 | 0.602 | 0.911 |
| 7 days | CD177 + IL1R2 + IFNG + TAP2 + CD3D + CTLA4 | 0.751 | 0.595 | 0.906 |
| 7 days | CD177 + IL1R2 + IFNG + TAP2 + CTLA4 | 0.759 | 0.606 | 0.912 |
| 7 days | CD177 + IL1R2 + S100A9 | 0.742 | 0.578 | 0.907 |
| 7 days | CD177 + IL1R2 + S100A9 + CD3D | 0.74 | 0.58 | 0.899 |
| 7 days | CD177 + IL1R2 + S100A9 + CD3D + CTLA4 | 0.742 | 0.583 | 0.901 |
| 7 days | CD177 + IL1R2 + S100A9 + CIITA | 0.82 | 0.681 | 0.959 |
| 7 days | CD177 + IL1R2 + S100A9 + CIITA + CD3D | 0.831 | 0.699 | 0.964 |
| 7 days | CD177 + IL1R2 + S100A9 + CIITA + CD3D + CTLA4 | 0.82 | 0.683 | 0.957 |
| 7 days | CD177 + IL1R2 + S100A9 + CIITA + CTLA4 | 0.814 | 0.672 | 0.956 |
| 7 days | CD177 + IL1R2 + S100A9 + CIITA + TAP2 | 0.823 | 0.686 | 0.96 |
| 7 days | CD177 + IL1R2 + S100A9 + CIITA + TAP2 + CD3D | 0.831 | 0.699 | 0.964 |
| 7 days | CD177 + IL1R2 + S100A9 + CIITA + TAP2 + CD3D + CTLA4 | 0.834 | 0.704 | 0.964 |
| 7 days | CD177 + IL1R2 + S100A9 + CIITA + TAP2 + CTLA4 | 0.809 | 0.665 | 0.952 |
| 7 days | CD177 + IL1R2 + S100A9 + CTLA4 | 0.737 | 0.572 | 0.902 |
| 7 days | CD177 + IL1R2 + S100A9 + CX3CR1 | 0.753 | 0.592 | 0.915 |
| 7 days | CD177 + IL1R2 + S100A9 + CX3CR1 + CD3D | 0.751 | 0.59 | 0.911 |
| 7 days | CD177 + IL1R2 + S100A9 + CX3CR1 + CD3D + CTLA4 | 0.748 | 0.587 | 0.909 |
| 7 days | CD177 + IL1R2 + S100A9 + CX3CR1 + CIITA | 0.823 | 0.681 | 0.964 |
| 7 days | CD177 + IL1R2 + S100A9 + CX3CR1 + CIITA + CD3D | 0.837 | 0.707 | 0.966 |
| 7 days | CD177 + IL1R2 + S100A9 + CX3CR1 + CIITA + CD3D + CTLA4 | 0.839 | 0.713 | 0.965 |
| 7 days | CD177 + IL1R2 + S100A9 + CX3CR1 + CIITA + CTLA4 | 0.828 | 0.69 | 0.966 |
| 7 days | CD177 + IL1R2 + S100A9 + CX3CR1 + CIITA + TAP2 | 0.823 | 0.68 | 0.966 |
| 7 days | CD177 + IL1R2 + S100A9 + CX3CR1 + CIITA + TAP2 + CD3D | 0.828 | 0.692 | 0.964 |
| 7 days | CD177 + IL1R2 + S100A9 + CX3CR1 + CIITA + TAP2 + CD3D + | 0.839 | 0.713 | 0.965 |
| | CTLA4 | | | |
| 7 days | CD177 + IL1R2 + S100A9 + CX3CR1 + CIITA + TAP2 + CTLA4 | 0.828 | 0.688 | 0.969 |
| 7 days | CD177 + IL1R2 + S100A9 + CX3CR1 + CTLA4 | 0.751 | 0.587 | 0.914 |
| 7 days | CD177 + IL1R2 + S100A9 + CX3CR1 + TAP2 | 0.765 | 0.604 | 0.925 |
| 7 days | CD177 + IL1R2 + S100A9 + CX3CR1 + TAP2 + CD3D | 0.765 | 0.606 | 0.923 |
| 7 days | CD177 + IL1R2 + S100A9 + CX3CR1 + TAP2 + CD3D + CTLA4 | 0.759 | 0.6 | 0.918 |
| 7 days | CD177 + IL1R2 + S100A9 + CX3CR1 + TAP2 + CTLA4 | 0.765 | 0.603 | 0.926 |
| 7 days | CD177 + IL1R2 + S100A9 + TAP2 | 0.748 | 0.587 | 0.908 |
| 7 days | CD177 + IL1R2 + S100A9 + TAP2 + CD3D | 0.753 | 0.598 | 0.909 |
| 7 days | CD177 + IL1R2 + S100A9 + TAP2 + CD3D + CTLA4 | 0.753 | 0.598 | 0.909 |
| 7 days | CD177 + IL1R2 + S100A9 + TAP2 + CTLA4 | 0.748 | 0.587 | 0.909 |
| 7 days | CD177 + IL1R2 + TAP2 | 0.734 | 0.568 | 0.9 |
| 7 days | CD177 + IL1R2 + TAP2 + CD3D | 0.729 | 0.561 | 0.896 |
| 7 days | CD177 + IL1R2 + TAP2 + CTLA4 | 0.731 | 0.563 | 0.9 |
| 7 days | CD177 + S100A9 + CD3D | 0.742 | 0.579 | 0.905 |
| 7 days | CD177 + S100A9 + CD3D + CTLA4 | 0.745 | 0.582 | 0.908 |
| 7 days | CD177 + S100A9 + CIITA | 0.792 | 0.639 | 0.946 |
| 7 days | CD177 + S100A9 + CIITA + CD3D | 0.798 | 0.652 | 0.943 |
| 7 days | CD177 + S100A9 + CIITA + CD3D + CTLA4 | 0.798 | 0.651 | 0.944 |
| 7 days | CD177 + S100A9 + CIITA + CTLA4 | 0.806 | 0.661 | 0.951 |
| 7 days | CD177 + S100A9 + CIITA + TAP2 | 0.792 | 0.639 | 0.946 |
| 7 days | CD177 + S100A9 + CIITA + TAP2 + CD3D | 0.798 | 0.652 | 0.943 |
| 7 days | CD177 + S100A9 + CIITA + TAP2 + CD3D + CTLA4 | 0.798 | 0.651 | 0.944 |
| 7 days | CD177 + S100A9 + CIITA + TAP2 + CTLA4 | 0.809 | 0.665 | 0.953 |
| 7 days | CD177 + S100A9 + CTLA4 | 0.748 | 0.584 | 0.912 |
| 7 days | CD177 + S100A9 + CX3CR1 | 0.751 | 0.587 | 0.914 |
| 7 days | CD177 + S100A9 + CX3CR1 + CD3D | 0.753 | 0.592 | 0.915 |
| 7 days | CD177 + S100A9 + CX3CR1 + CD3D + CTLA4 | 0.753 | 0.592 | 0.915 |
| 7 days | CD177 + S100A9 + CX3CR1 + CIITA | 0.834 | 0.695 | 0.973 |
| 7 days | CD177 + S100A9 + CX3CR1 + CIITA + CD3D | 0.825 | 0.683 | 0.968 |
| 7 days | CD177 + S100A9 + CX3CR1 + CIITA + CD3D + CTLA4 | 0.82 | 0.68 | 0.96 |
| 7 days | CD177 + S100A9 + CX3CR1 + CIITA + CTLA4 | 0.825 | 0.685 | 0.966 |
| 7 days | CD177 + S100A9 + CX3CR1 + CIITA + TAP2 | 0.823 | 0.68 | 0.966 |
| 7 days | CD177 + S100A9 + CX3CR1 + CIITA + TAP2 + CD3D | 0.82 | 0.676 | 0.964 |
| 7 days | CD177 + S100A9 + CX3CR1 + CIITA + TAP2 + CD3D + CTLA4 | 0.817 | 0.676 | 0.958 |
| 7 days | CD177 + S100A9 + CX3CR1 + CIITA + TAP2 + CTLA4 | 0.82 | 0.677 | 0.963 |
| 7 days | CD177 + S100A9 + CX3CR1 + CTLA4 | 0.751 | 0.587 | 0.914 |
| 7 days | CD177 + S100A9 + CX3CR1 + TAP2 | 0.765 | 0.604 | 0.925 |
| 7 days | CD177 + S100A9 + CX3CR1 + TAP2 + CD3D | 0.765 | 0.604 | 0.925 |
| 7 days | CD177 + S100A9 + CX3CR1 + TAP2 + CD3D + CTLA4 | 0.765 | 0.604 | 0.925 |
| 7 days | CD177 + S100A9 + CX3CR1 + TAP2 + CTLA4 | 0.767 | 0.607 | 0.928 |
| 7 days | CD177 + S100A9 + TAP2 | 0.737 | 0.571 | 0.903 |
| 7 days | CD177 + S100A9 + TAP2 + CD3D | 0.737 | 0.571 | 0.903 |
| 7 days | CD177 + S100A9 + TAP2 + CD3D + CTLA4 | 0.745 | 0.581 | 0.91 |
| 7 days | CD177 + S100A9 + TAP2 + CTLA4 | 0.737 | 0.571 | 0.903 |
| 7 days | CD177 + TAP2 + CTLA4 | 0.731 | 0.562 | 0.9 |

Table 7 Performance (AUC and 95% confidence interval, with AUC2.5 and AUC 97.5 limits) of the measurement of the expression of CD177 in combination with one or more other biomarker(s) (multivariate analysis) in predicting the incidence of a healthcare-associated infection in the 4 days or in the 7 days following the sample being taken.

1. An in vitro or ex vivo method for determining the risk of incidence of a healthcare-associated infection in a patient, comprising a step of measuring the expression of CD177 in a biological sample from said patient.

2. The method as claimed in claim 1, wherein it additionally comprises a step of measuring, in the biological sample from the patient, the expression:
of at least one gene selected from the family of genes encoding molecules involved in the innate immune system, and/or
of at least one gene selected from the family of genes encoding molecules of the cell cycle, and/or
of at least one gene selected from the family of genes encoding cytokines, and/or
of at least one gene selected from the family of genes encoding anti-inflammatory cytokines, and/or
of at least one gene selected from the family of genes encoding pro-inflammatory cytokine receptors, located on chromosome 2 in the the region 2q11-2q12, and/or
of at least one gene selected from the family of genes encoding pro-inflammatory cytokines, and/or
of at least one gene selected from the family of genes encoding molecules involved in cytoskeleton formation, and/or
of at least one gene selected from the family of genes encoding molecules involved in gene expression and/or transcription, and/or
of at least one gene selected from the family of genes encoding growth factors, and/or
of at least one gene selected from the family of genes encoding molecules of the metabolism, and/or
of at least one gene selected from the family of genes encoding molecules involved in the adaptive immune system, and/or
of at least one gene selected from the family of genes encoding molecules involved in signal transduction, and/or
of at least one gene selected from the family of genes encoding molecules involved in modulation of the acute phase.

3. The method as claimed in claim 1, wherein it comprises a step of measuring, in the biological sample from the patient, the expression:
of at least one gene selected from the following genes: GNLY, S100A9, C3AR1, ADGRE3, CX3CR1, IFIH1, OAS2, OAS3, and/or
of the CCNB1IP1 gene, and/or
of at least one gene selected from the following genes: IL15, IL2, MCP1(CCL2), CXCL10, and/or
of at least one gene selected from the following genes: IL10, IL1RN, and/or
of at least one gene selected from the following genes: IL18R1, IL1R2, IL1R1 and IL18RAP, and/or
of at least one gene selected from the following genes: IFNG, IL1B, IL17A, IL18, IL6, TNF, and/or
of at least one gene selected from the following genes: ARL14EP, GSN, and/or
of at least one gene selected from the following genes: CIITA, DYRK2, GATA3, MDC1, NFKB1, RORC, STAT4, TBX21, TDRD9, and/or
of the CSF2 gene, and/or
of at least one gene selected from the following genes: ALOX5, BPGM, TRAP1, and/or
of at least one gene selected from the following genes: CD40LG, CD3D, BTLA, CD274, CTLA4, ICOS, PDCD1, TNFSF4, CD74, FCGR1A, LILRB2, TAP2, and/or
of at least one gene selected from the following genes: FLT1, HAVCR2, IL7R, ZAP70, and/or
of the HP gene.

4. The method as claimed in claim 1, wherein it comprises a step of measuring, in the biological sample from the patient, the expression of at least one gene selected from the following genes: GNLY, S100A9, C3AR1, ADGRE3, TAP2, CX3CR1, OAS2, CCNB1IP1, IL10, IL1RN, IL1R2, IFNG, TNF, ARL14EP, CIITA, GATA3, MDC1, TDRD9, BPGM, CD3D, CD274, CTLA4, CD74, IL7R, ZAP70, HP.

5. The method as claimed in claim 4, wherein it comprises a step of measuring, in the biological sample from the patient, the expression of at least one gene selected from the following genes: S100A9, C3AR1, TAP2, CX3CR1, IL1R2, IFNG, CIITA, CD3D, CTLA4, CD74, HP.

6. The method as claimed in, claim 1, wherein the biological sample is a blood sample.

7. The method as claimed in claim 1, wherein the expression is measured at the messenger RNA (mRNA) level.

8. The method as claimed in claim 1, wherein the expression is measured by RT-PCR.

9. The method as claimed in claim 1, wherein the expression is measured by sequencing.

10. The method as claimed in claim 1, wherein the expression is measured by hybridization.

11. The method as claimed in claim 7, wherein the expression is normalized in relation to the expression of one or more housekeeping genes.

12. A kit comprising means for amplifying and/or means for detecting the expression of CD177 and of at least one other gene, selected from the genes of claim 2; said kit wherein all the amplification and/or detection means of said kit enable the detection and/or amplification of at most 100 biomarkers in total.

13. A method comprising using in vitro or ex vivo:
of means for amplifying and/or means for detecting the expression of CD177, or
of a kit comprising such amplification and/or detection means,
for determining the risk of incidence of a healthcare-associated infection in a patient.
